(19) United States
(12) Patent Application Publication
(10) Pub. No.: US 2003/0235820 A1
(43) Pub. Date: Dec. 25, 2003

(54) Novel methods of diagnosis of metastatic colorectal cancer, compositions and methods of screening for modulators of metastatic colorectal cancer

(75) Inventors: David H. Mack, Menlo Park, CA (US); Sanford David Markowitz, Pepper Pike, OH (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Eos Biotechnology, Inc., South San Francisco, CA

(21) Appl. No.: 10/087,080

(22) Filed: Feb. 27, 2002

Related U.S. Application Data
(60) Provisional application No. 60/284,555, filed on Apr. 17, 2001.
(60) Provisional application No. 60/281,149, filed on Apr. 2, 2001.
(60) Provisional application No. 60/272,206, filed on Feb. 27, 2001.

(51) Int. Cl. C12Q 1/68; G01N 33/574; C07H 21/04; C12N 9/00; C12P 21/02; C12N 5/06; C07K 16/40

(52) U.S. Cl. 435/6; 435/69.1; 435/183; 435/320.1; 435/325; 530/388.26; 536/23.2; 435/7.23

(57) ABSTRACT

Described herein are methods and compositions that can be used for diagnosis and treatment of metastatic colorectal cancer. Also described herein are methods that can be used to identify modulators of metastatic colorectal cancer.

### CROSS-REFERENCES TO RELATED APPLICATIONS

The present application is related to U.S. S No. 60/272,206, filed Feb. 27, 2001, U.S. S No. 60/281,149, filed Apr. 2, 2001, and U.S. S No. 60/284,555, filed Apr. 17, 2001, all of which are herein incorporated by referenced in their entirety.

### FIELD OF THE INVENTION

The invention relates to the identification of nucleic acid and protein expression profiles and nucleic acids, products, and antibodies thereto that are involved in metastatic colorectal cancer; and to the use of such expression profiles and compositions in diagnosis and therapy of metastatic colorectal cancer. The invention further relates to methods for identifying and using agents and/or targets that inhibit metastatic colorectal cancer.

### BACKGROUND OF THE INVENTION

Cancer of the colon and/or rectum (referred to as “colorectal cancer”) are significant in Western populations and particularly in the United States. Cancers of the colon and rectum occur in both men and women most commonly after the age of 50. These develop as the result of a pathologic transformation of normal colon epithelium to an invasive cancer. There have been a number of recently characterized genetic alterations that have been implicated in colorectal cancer, including mutations in two classes of genes, tumor-suppressor genes and proto-oncogenes, with recent work suggesting that mutations in DNA repair genes may also be involved in tumorigenesis. For example, inactivating mutations of both alleles of the adenomatous polyposis coli (APC) gene, a tumor suppressor gene, appears to be one of the earliest events in colorectal cancer, and may even be the initiating event. Other genes implicated in colorectal cancer include the MCC gene, the p53 gene, the DCC (deleted in colorectal carcinoma) gene and other chromosome 18q genes, and genes in the TGF-β signaling pathway. For a review, see Molecular Biology of Colorectal Cancer, pp. 238-299, in Curr. Probl. Cancer, September/October 1997; see also Willams, Colorectal Cancer (1996); Kinsella & Schofield, Colorectal Cancer: A Scientific Perspective (1993); Colorectal Cancer: Molecular Mechanisms, Premalignant State and its Prevention (Schiniegel & Scholmerich eds., 2000); Colorectal Cancer: New Aspects of molecular Biology and Their Clinical Applications (Hanski et al., eds 2000); McArdle et al., Colorectal Cancer (2000); Wanebo, Colorectal Cancer (1993); Levin, The American Cancer Society: Colorectal Cancer (1999); Treatment of Hepatic Metastases of Colorectal Cancer (Nordlinger & Jaeck eds., 1993); Management of Colorectal Cancer (Dunitz et al., eds. 1998); Cancer: Principles and Practice of Oncology (Devita et al., eds. 2001); Surgical Oncology: Contemporary Principles and Practice (Kirby et al., eds. 2001); Offit, Clinical Cancer Genetics: Risk Counseling and Management (1997); Radioimmunotherapy of Cancer (Abrams & Fritzberg eds. 2000); Fleming, AJCC Cancer Staging Handbook (1998); Textbook of Radiation Oncology (Leibel & Phillips eds. 2000); and Clinical Oncology (Abeloff et al., eds. 2000).

Imaging of colorectal cancer for diagnosis has been problematic and limited.

In addition, metastasis of the tumor to the lumen, and metastasis of tumor cells to regional lymph nodes are important prognostic factors (see, e.g., PET in Oncology: Basics and Clinical Application (Ruhlmann et al. eds. 1999). For example, five year survival rates drop from 80 percent in patients with no lymph node metastases to 45 to 50 percent in those patients who do have lymph node metastases. A recent report showed that micrometastases can be detected from lymph nodes using reverse transcriptase-PCR methods based on the presence of mRNA for carcinoembryonic antigen, which has previously been shown to be present in the vast majority of colorectal cancers but not in normal tissues. Liefers et al., New England J. of Med. 339(4):223 (1998). In addition, colorectal cancers often metastasize to the liver. However, the lack of information about the gene expression exhibited by these cancers limits the ability to effectively diagnose and treat the disease. Thus, methods for diagnosis and prognosis of metastatic colorectal cancer and effective treatment of colorectal cancer would be desirable. Accordingly, provided herein are methods that can be used in diagnosis and prognosis of metastatic colorectal cancer. Further provided are methods that can be used to screen candidate therapeutic agents for the ability to modulate, e.g., treat, colorectal cancer. Additionally, provided herein are molecular targets and compositions for therapeutic intervention in metastatic colorectal disease and other metastatic cancers.

### SUMMARY OF THE INVENTION

The present invention therefore provides nueleotide sequences of genes that are up- and down-regulated in metastatic colorectal cancer cells. Such genes and the proteins they encode are useful for diagnostic and prognostic purposes, and also as targets for screening for therapeutic compounds that modulate metastatic colorectal cancer, such as antibodies. The methods of detecting nucleic acids of the invention or their encoded proteins can be used for a number of purposes. Examples include, early detection of colon cancers, monitoring and early detection of relapse following treatment of colon cancers, monitoring response to therapy of colon cancers, determining prognosis of colon cancers, directing therapy of colon cancers, selecting patients for postoperative chemotherapy or radiation therapy, selecting therapy, determining tumor prognosis, treatment, or response to treatment, and early detection of precancerous colon adenomas. Other aspects of the invention will become apparent to the skilled artisan by the following description of the invention.

In one aspect, the present invention provides a method of detecting a metastatic colorectal cancer-associated transcript in a cell from a patient, the method comprising contacting a biological sample from the patient with a polynucleotide that selectively hybridizes to a sequence at least 80% identical to a sequence as shown in Tables 1-26.

In one embodiment, the polynucleotide selectively hybridizes to a sequence at least 95% identical to a sequence as shown in Tables 1-26. In another embodiment, the polynucleotide comprises a sequence as shown in Tables 1-26.

In one embodiment, the biological sample is a tissue sample. In another embodiment, the biological sample comprises isolated nucleic acids, e.g., mRNA.

In one embodiment, the polynucleotide is labeled, e.g., with a fluorescent label.

In one embodiment, the polynucleotide is immobilized on a solid surface.

In one embodiment, the patient is undergoing a therapeutic regimen to treat metastatic colorectal cancer. In another embodiment, the patient is suspected of having metastatic colorectal cancer.

In one embodiment, the patient is a human.

In one embodiment, the method further comprises the step of amplifying nucleic acids before the step of contacting the biological sample with the polynucleotide.

In another aspect, the present invention provides methods of detecting polypeptide encoded by a metastatic colorectal cancer-associated transcript in a cell from a patient, the method comprising contacting a biological sample from the patient with an antibody that specifically binds a polypeptide encoded by a sequence at least 80% identical to a sequence as shown in Tables 1-26.

In another aspect, the present invention provides a method of monitoring the efficacy of a therapeutic treatment of metastatic colorectal cancer, the method comprising the steps of: (i) providing a biological sample from a patient undergoing the therapeutic treatment; and (ii) determining the level of a metastatic colorectal cancer-associated transcript in the biological sample by contacting the biological sample with a polynucleotide that selectively hybridizes to a sequence at least 80% identical to a sequence as shown in Tables 1-26., thereby monitoring the efficacy of the therapy.

In one embodiment, the method further comprises the step of: (iii) comparing the level of the metastatic colorectal cancer-associated transcript to a level of the metastatic colorectal cancer-associated transcript in a biological sample from the patient prior to, or earlier in, the therapeutic treatment.

In another aspect, the present invention provides a method of monitoring the efficacy of a therapeutic treatment of metastatic colorectal cancer, the method comprising the steps of: (i) providing a biological sample from a patient undergoing the therapeutic treatment; and (ii) determining the level of a metastatic colorectal cancer-associated antibody in the biological sample by contacting the biological sample with a polypeptide encoded by a polynucleotide that selectively hybridizes to a sequence at least 80% identical to a sequence as shown in Tables 1-26, wherein the polypeptide specifically binds to the metastatic colorectal cancer-associated antibody, thereby monitoring the efficacy of the therapy.

In one embodiment, the method further comprises the step of: (iii) comparing the level of the metastatic colorectal cancer-associated antibody to a level of the metastatic colorectal cancer-associated antibody in a biological sample from the patient prior to, or earlier in, the therapeutic treatment.

In another aspect, the present invention provides a method of monitoring the efficacy of a therapeutic treatment of metastatic colorectal cancer, the method comprising the steps of: (i) providing a biological sample from a patient undergoing the therapeutic treatment; and (ii) determining the level of a metastatic colorectal cancer-associated polypeptide in the biological sample by contacting the biological sample with an antibody, wherein the antibody specifically binds to a polypeptide encoded by a polynucleotide that selectively hybridizes to a sequence at least 80% identical to a sequence as shown in Tables 1-26, thereby monitoring the efficacy of the therapy.

In one embodiment, the method further comprises the step of: (iii) comparing the level of the metastatic colorectal cancer-associated polypeptide to a level of the metastatic colorectal cancer-associated polypeptide in a biological sample from the patient prior to, or earlier in, the therapeutic treatment.

In one aspect, the present invention provides an isolated nucleic acid molecule consisting of a polynucleotide sequence as shown in Tables 1-26.

In one embodiment, an expression vector or cell comprises the isolated nucleic acid.

In one aspect, the present invention provides an isolated polypeptide which is encoded by a nucleic acid molecule having polynucleotide sequence as shown in Tables 1-26.

In another aspect, the present invention provides an antibody that specifically binds to an isolated polypeptide which is encoded by a nucleic acid molecule having polynucleotide sequence as shown in Tables 1-26.

In one embodiment, the antibody is conjugated to an effector component, e.g., a fluorescent label, a radioisotope or a cytotoxic chemical.

In one embodiment, the antibody is an antibody fragment. In another embodiment, the antibody is humanized.

In one aspect, the present invention provides a method of detecting a metastatic colorectal cancer cell in a biological sample from a patient, the method comprising contacting the biological sample with an antibody as described herein.

In another aspect, the present invention provides a method of detecting antibodies specific to metastatic colorectal cancer in a patient, the method comprising contacting a biological sample from the patient with a polypeptide encoded by a nucleic acid comprises a sequence from Tables 1-26.

In another aspect, the present invention provides a method for identifying a compound that modulates a metastatic colorectal cancer-associated polypeptide, the method comprising the steps of: (i) contacting the compound with a metastatic colorectal cancer-associated polypeptide, the polypeptide encoded by a polynucleotide that selectively hybridizes to a sequence at least 80% identical to a sequence as shown in Tables 1-26; and (ii) determining the functional effect of the compound upon the polypeptide.

In one embodiment, the functional effect is a physical effect, an enzymatic effect, or a chemical effect.

In one embodiment, the polypeptide is expressed in a eukaryotic host cell or cell membrane. In another embodiment, the polypeptide is recombinant.

In one embodiment, the functional effect is determined by measuring ligand binding to the polypeptide.

In another aspect, the present invention provides a method of inhibiting proliferation of a metastatic colorectal cancer-associated cell to treat colorectal cancer in a patient, the method comprising the step of administering to the subject a therapeutically effective amount of a compound identified as described herein.

In one embodiment, the compound is an antibody.

In another aspect, the present invention provides a drug screening assay comprising the steps of: (i) administering a test compound to a mammal having colorectal cancer or a cell isolated therefrom; (ii) comparing the level of gene expression of a polynucleotide that selectively hybridizes to a sequence at least 80% identical to a sequence as shown in Tables 1-26. in a treated cell or mammal with the level of gene expression of the polynucleotide in a control cell or mammal, wherein a test compound that modulates the level of expression of the polynucleotide is a candidate for the treatment of colorectal cancer.

In one embodiment, the control is a mammal with colorectal cancer or a cell therefrom that has not been treated with the test compound. In another embodiment, the control is a normal cell or mammal.

In another aspect, the present invention provides a method for treating a mammal having colorectal cancer comprising administering a compound identified by the assay described herein.

In another aspect, the present invention provides a pharmaceutical composition for treating a mammal having colorectal cancer, the composition comprising a compound identified by the assay described herein and a physiologically acceptable excipient.

### DETAILED DESCRIPTION OF THE INVENTION

In accordance with the objects outlined above, the present invention provides novel methods for diagnosis and treatment of colon and/or rectal cancer (e.g. colorectal cancer), including metastatic colorectal cancers, as well as methods for screening for compositions which modulate colorectal cancer. By “metastatic colorectal cancer” herein is meant a colon and/or rectal tumor or cancer that is classified as Dukes stage C or D (see, e.g., Cohen et al., Cancer of the Colon, in Cancer: Principles and Practice of Oncology, pp. 1144-1197 (Devita et al., eds., 5^{th} ed. 1997); see also Harrison 's Principles of Internal Medicine, pp. 1289-129 (Wilson et al., eds., 12^{th} ed., 1991). “Treatment, monitoring, detection or modulation of metastatic colorectal cancer” includes treatment, monitoring, detection, or modulation of metastatic colorectal disease in those patients who have metastatic colorectal disease (Dukes stage C or D). In Dukes stage A, the tumor has penetrated into, but not through, the bowel wall. In Dukes stage B, the tumor has penetrated through the bowel wall but there is not yet any lymph involvement. In Dukes stage C, the cancer involves regional lymph nodes. In Dukes stage D, there is distant metastasis, e.g., liver, lung, etc.

Tables 1-26 provide UniGene cluster identification numbers for the nucleotide sequence of genes that exhibit increased or decreased expression in metastasizing colorectal cancer samples. Tables 1-26 also provide an exemplar accession number that provides a nucleotide sequence that is part of the UniGene cluster. In Tables 1-26, the ratio provided represents primary tumor samples from known Dukes B stage survivors vs. liver metastasis samples from patients with metastatic colorectal cancer. In these samples, the identified genes are underexpressed in the metastatic samples, as the ratio is greater than one, preferably 1.5 or greater, more preferably 2.0 or greater. In Tables 1-26, the ratio provided represents liver metastasis samples from patients with known metastatic colorectal cancer vs. known primary tumor samples from Dukes B stage survivors. In these samples, the identified genes are overexpressed in the metastatic samples, as the ratio is greater than one, preferably 1.5 or greater, more preferably 2.0 or greater. In Tables 1-26, the ratio provided represents primary tumor samples from known Dukes B stage survivors vs. liver metastasis samples from patients with metastatic colorectal cancer. In these samples, the identified genes are overexpressed in the metastatic samples, as the ratio is less than one, preferably 0.5 or less, more preferably 0.25 or less. Survivors are subjects who have been disease free for five years or longer.

In Tables 1-26, the ratio provided represents liver metastasis samples from patients with known metastatic disease vs. tissue samples from normal colon tissue. In these samples, the identified genes are overexpressed in the metastatic samples, as the ratio is greater than one, preferably 1.5 or greater, more preferably 2.0 or greater. In Tables 1-26, the ratio represents liver metastasis samples from patients with known metastatic disease vs. tissue samples from normal colon tissue. In these samples, the identified genes are underexpressed in the metastatic samples, as the ratio is less than one, preferably 0.5 or less, more preferably 0.25 or less.

One of skill will recognize that although the sequences identified in Tables 1-26 exhibited increased or decreased expression in metastasizing colorectal cancer samples, the sequences of the invention, and their encoded proteins, can be used to diagnose, treat or prevent cancers in patients with Dukes stage A or B colorectal cancers. Alteration of gene expression for a gene in Tables 1-26 may be more likely or less likely to indicate that the subject will progress to metastatic disease. The sequences can also be used to diagnose, treat or prevent precancerous or benign conditions such as precancerous colon adenomas. Alteration of gene expression for a gene in Tables 1-26 may or may not indicate that the subject is more likely to progress to cancer or to metastatic disease. Thus, although the specification focuses primarily on metastasizing colorectal cancer, the methods described below can also be applied to non-metastasizing colorectal cancers (e.g., Dukes stages A and B) and precancerous or benign conditions (e.g., precancerous adenomas) as well.

Definitions

The term “metastatic colorectal cancer protein” or “metastatic colorectal cancer polynucleotide” or “metastatic colorectal cancer-associated transcript” refers to nucleic acid and polypeptide polymorphic variants, alleles, mutants, and interspecies homologs that: (1) have a nucleotide sequence that has greater than about 60% nucleotide sequence identity, 65%, 70%, 75%, 80%, 85%, 90%, preferably 91%, 92%, 93%, 94%, 95%, 96%, 97%, 98% or 99% or greater nucleotide sequence identity, preferably over a region of over a region of at least about 25, 50, 100, 200, 500, 1000, or more nucleotides, to a nucleotide sequence of or associated with a UniGene cluster of Tables 1-26; (2) bind to antibodies, e.g., polyclonal antibodies, raised against an immunogen comprising an amino acid sequence encoded by a nucleotide sequence of or associated with a UniGene cluster of Tables 1-26, and conservatively modified variants thereof; (3) specifically hybridize under stringent hybridization conditions to a nucleic acid sequence, or the complement thereof of Tables 1-26 and conservatively modified variants thereof or (4) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino sequence identity, preferably over a region of over a region of at least about 25, 50, 100, 200, 500, 1000, or more amino acid, to an amino acid sequence encoded by a nucleotide sequence of or associated with a UniGene cluster of Tables 1-26. A polynucleotide or polypeptide sequence is typically from a mammal including, but not limited to, primate, e.g., human; rodent, e.g., rat, mouse, hamster; cow, pig, horse, sheep, or other mammal. A “metastatic colorectal cancer polypeptide” and a “metastatic colorectal cancer polynucleotide,” include both naturally occurring or recombinant.

A “fill length” metastatic colorectal cancer protein or nucleic acid refers to a metastatic colorectal cancer polypeptide or polynucleotide sequence, or a variant thereof, that contains all of the elements normally contained in one or more naturally occurring, wild type metastatic colorectal cancer polynucleotide or polypeptide sequences. The “full length” may be prior to, or after, various stages of post-translation processing or splicing, including alternative splicing.

“Biological sample” as used herein is a sample of biological tissue or fluid that contains nucleic acids or polypeptides, e.g., of a metastatic colorectal cancer protein, polynucleotide or transcript. Such samples include, but are not limited to, tissue isolated from primates, e.g., humans, or rodents, e.g., mice, and rats. Biological samples may also include sections of tissues such as biopsy and autopsy samples, frozen sections taken for histologic purposes, blood, plasma, serum, sputum, stool, tears, mucus, hair, skin, etc. Biological samples also include explants and primary and/or transformed cell cultures derived from patient tissues. A biological sample is typically obtained from a eukaryotic organism, most preferably a mammal such as a primate, e.g., chimpanzee or human; cow; dog; cat; a rodent, e.g., guinea pig, rat, mouse; rabbit; or other mammal; or a bird; reptile; fish.

“Providing a biological sample” means to obtain a biological sample for use in methods described in this invention. Most often, this will be done by removing a sample of cells from an animal, but can also be accomplished by using previously isolated cells (e.g., isolated by another person, at another time, and/or for another purpose), or by performing the methods of the invention in vivo. Archival tissues, having treatment or outcome history, will be particularly useful.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g. NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff& Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90, 110, 150, 170, etc.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

A “host cell” is a naturally occurring cell or a transformed cell that contains an expression vector and supports the replication or expression of the expression vector. Host cells may be cultured cells, explants, cells in vivo, and the like. Host cells may be prokaryotic cells such as E. coli, or eukaryotic cells such as yeast, insect, amphibian, or mammalian cells such as CHO, HeLa, and the like (see, e.g., the American Type Culture Collection catalog or web site, www.atcc.org).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are typically conservative substitutions for one another: 1) Alanine (A), Glycine (G); 2) Aspartic acid (D), Glutamic acid (E); 3) Asparagine (N), Glutamine (O); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor & Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that often form a compact unit of the polypeptide and are typically 25 to approximately 500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed, usually by the noncovalent association of independent tertiary units. Anisotropic terms are also known as energy terms.

“Nucleic acid” or “oligonucleotide” or “polynucleotide” or grammatical equivalents used herein means at least two nucleotides covalently linked together. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. A nucleic acid of the present invention will generally contain phosphodiester bonds, although in some cases, nucleic acid analogs are included that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g. to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tₘ) for mismatched versus perfectly matched basepairs. DNA and RNA typically exhibit a 2-4° C. drop in Tₘ for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

The nucleic acids may be single stranded or double stranded, as specified, or contain portions of both double stranded or single stranded sequence. As will be appreciated by those in the art, the depiction of a single strand also defines the sequence of the complementary strand; thus the sequences described herein also provide the complement of the sequence. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. “Transcript” typically refers to a naturally occurring RNA, e.g., a pre-mRNA, hnRNA, or mRNA. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g. the individual units of a peptide nucleic acid, each containing a base, are referred to herein as a nucleoside.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide.

An “effector” or “effector moiety” or “effector component” is a molecule that is bound (or linked, or conjugated), either covalently, through a linker or a chemical bond, or noncovalently, through ionic, van der Waals, electrostatic, or hydrogen bonds, to an antibody. The “effector” can be a variety of molecules including, e.g., detection moieties including radioactive compounds, fluorescent compounds, an enzyme or substrate, tags such as epitope tags, a toxin; activatable moieties, a chemotherapeutic agent; a lipase; an antibiotic; or a radioisotope emitting “hard” e.g., beta radiation.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, method using high affinity interactions may achieve the same results where one of a pair of binding partners binds to the other, e.g., biotin, streptavidin.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not functionally interfere with hybridization. Thus, e.g., probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence. Diagnosis or prognosis may be based at the genomic level, or at the level of RNA or protein expression.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to essentially no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions are often: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec—2 min., an annealing phase lasting 30 sec.—2 min., and an extension phase of about 72° C. for 1-2 min. Protocols and guidelines for low and high stringency amplification reactions are provided, e.g., in Innis et al., PCR Protocols, A Guide to Methods and Applications (1990).

Nucleic acids that do not hybridize to each other under stringent conditions are still substantially identical if the polypeptides which they encode are substantially identical. This occurs, e.g., when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

The phrase “functional effects” in the context of assays for testing compounds that modulate activity of a metastatic colorectal cancer protein includes the determination of a parameter that is indirectly or directly under the influence of the metastatic colorectal cancer protein or nucleic acid, e.g., an enzymatic, functional, physical, or chemical effect, such as the ability to decrease metastatic colorectal cancer. It includes ligand binding activity; cell growth on soft agar; anchorage dependence; contact inhibition and density limitation of growth; cellular proliferation; cellular transformation; growth factor or serum dependence; tumor specific marker levels; invasiveness into Matrigel; tumor growth and metastasis in vivo; mRNA and protein expression in cells undergoing metastasis, and other characteristics of metastatic colorectal cancer cells. “Functional effects” include in vitro, in vivo, and ex vivo activities.

By “determining the functional effect” is meant assaying for a compound that increases or decreases a parameter that is indirectly or directly under the influence of a metastatic colorectal cancer protein sequence, e.g., functional, enzymatic, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties for the protein, measuring inducible markers or transcriptional activation of the metastatic colorectal cancer protein; measuring binding activity or binding assays, e.g., binding to antibodies or other ligands, and measuring cellular proliferation. Determination of the functional effect of a compound on metastatic colorectal cancer can also be performed using metastatic colorectal cancer assays known to those of skill in the art such as an in vitro assays, e.g., cell growth on soft agar; anchorage dependence; contact inhibition and density limitation of growth; cellular proliferation; cellular transformation; growth factor or serum dependence; tumor specific marker levels; invasiveness into Matrigel; tumor growth and metastasis in vivo; mRNA and protein expression in cells undergoing metastasis, and other characteristics of metastatic colorectal cancer cells. The functional effects can be evaluated by many means known to those skilled in the art, e.g., microscopy for quantitative or qualitative measures of alterations in morphological features, measurement of changes in RNA or protein levels for metastatic colorectal cancer-associated sequences, measurement of RNA stability, identification of downstream or reporter gene expression (CAT, luciferase, β-gal, GFP and the like), e.g., via chemiluminescence, fluorescence, colorimetric reactions, antibody binding, inducible markers, and ligand binding assays.

“Ihiibitors”, “activators”, and “modulators” of metastatic colorectal cancer polynucleotide and polypeptide sequences are used to refer to activating, inhibitory, or modulating molecules or compounds identified using in vitro and in vivo assays of metastatic colorectal cancer polynucleotide and polypeptide sequences of the invention. Inhibitors are compounds that, e.g., bind to, partially or totally block activity, decrease, prevent, delay activation, inactivate, desensitize, or down regulate the activity or expression of metastatic colorectal cancer proteins of the invention, e.g., antagonists. Antisense nucleic acids may seem to inhibit expression and subsequent function of the protein. “Activators” are compounds that increase, open, activate, facilitate, enhance activation, sensitize, agonize, or up regulate metastatic colorectal cancer protein activity. Inhibitors, activators, or modulators also include genetically modified versions of metastatic colorectal cancer proteins, e.g., versions with altered activity, as well as naturally occurring and synthetic ligands, antagonists, agonists, antibodies, small chemical molecules and the like. Such assays for inhibitors and activators include, e.g., expressing the metastatic colorectal cancer protein in vitro, in cells, or cell membranes, applying putative modulator compounds, and then determining the functional effects on activity, as described above. Activators and inhibitors of metastatic colorectal cancer can also be identified by incubating metastatic colorectal cancer cells with the test compound and determining increases or decreases in the expression of 1 or more metastatic colorectal cancer proteins, e.g., 1, 2, 3, 4, 5, 10, 15, 20, 25, 30, 40, 50 or more metastatic colorectal cancer proteins, such as colorectal cancer proteins encoded by the sequences set out in Tables 1-26.

Samples or assays comprising metastatic colorectal cancer proteins that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of a polypeptide is achieved when the activity value relative to the control is about 80%, preferably 50%, more preferably 25-0%. Activation of a metastatic colorectal cancer polypeptide is achieved when the activity value relative to the control (untreated with activators) is 110%, more preferably 150%, more preferably 200-500% (i.e., two to five fold higher relative to the control), more preferably 1000-3000% higher.

The phrase “changes in cell growth” refers to any change in cell growth and proliferation characteristics in vitro or in vivo, such as formation of foci, anchorage independence, semi-solid or soft agar growth, changes in contact inhibition and density limitation of growth, loss of growth factor or serum requirements, changes in cell morphology, gaining or losing immortalization, gaining or losing tumor specific markers, ability to form or suppress tumors when injected into suitable animal hosts, and/or immortalization of the cell. See, e.g., Freshney, Culture of Animal Cells a Manual of Basic Technique pp. 231-241 (3^{rd} ed. 1994).

“Tumor cell” refers to precancerous, cancerous, and normal cells in a tumor.

“Cancer cells,” “transformed” cells or “transformation” in tissue culture, refers to spontaneous or induced phenotypic changes that do not necessarily involve the uptake of new genetic material. Although transformation can arise from infection with a transforming virus and incorporation of new genomic DNA, or uptake of exogenous DNA, it can also arise spontaneously or following exposure to a carcinogen, thereby mutating an endogenous gene. Transformation is associated with phenotypic changes, such as immortalization of cells, aberrant growth control, nonmorphological changes, and/or malignancy (see, Freshney, Culture of Animal Cells a Manual of Basic Technique (3^{rd} ed. 1994)).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody or its functional equivalent will be most critical in specificity and affinity of binding. See Paul, Fundamental Immunology.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, e.g., pepsin digests an antibody below the disulfide linkages in the hinge region to produce F(ab′)₂, a dimer of Fab which itself is a light chain joined to V_{H}-C_{H}1 by a disulfide bond. The F(ab′)₂ may be reduced under mild conditions to break the disulfide linkage in the hinge region, thereby converting the F(ab′)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g. Kohler & Milstein, Nature 256:495-497 (1975); Kozbor et al., Immunology Today 4:72 (1983); Cole et al., pp. 77-96 in Monoclonal Antibodies and Cancer Therapy (1985); Coligan, Current Protocols in Immunology (1991); Harlow & Lane, Antibodies, A Laboratory Manual (1988); and Goding, Monoclonal Antibodies: Principles and Practice (2d ed. 1986)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which, e.g, (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

Identification of Metastatic Colorectal Cancer-Associated Sequences

In one aspect, the expression levels of genes are determined in different patient samples for which diagnosis information is desired, to provide expression profiles. An expression profile of a particular sample is essentially a “fingerprint” of the state of the .sample; while two states may have any particular gene similarly expressed, the evaluation of a number of genes simultaneously allows the generation of a gene expression profile that is characteristic of the state of the cell. That is, normal tissue may be distinguished from cancerous or metastatic cancerous tissue, or metastatic cancerous tissue can be compared with tissue from surviving cancer patients. By comparing expression profiles of tissue in known different metastatic colorectal cancer states, information regarding which genes are important (including both up- and down-regulation of genes) in each of these states is obtained.

The identification of sequences that are differentially expressed in metastatic colorectal cancer versus non-metastatic colorectal cancer tissue allows the use of this information in a number of ways. For example, a particular treatment regime may be evaluated: does a chemotherapeutic drug act to down-regulate metastatic colorectal cancer, and thus tumor growth or recurrence, in a particular patient. Similarly, diagnosis and treatment outcomes may be done or confirmed by comparing patient samples with the known expression profiles. Metastatic tissue can also be analyzed to determine the stage of metastatic colorectal cancer in the tissue. Furthermore, these gene expression profiles (or individual genes) allow screening of drug candidates with an eye to mimicking or altering a particular expression profile; e.g., screening can be done for drugs that suppress the metastatic colorectal cancer expression profile. This may be done by making biochips comprising sets of the important metastatic colorectal cancer genes, which can then be used in these screens. PCR methods may be applied with selected primer pairs, and analysis may be of RNA or of genomic sequences. These methods can also be done on the protein basis; that is, protein expression levels of the metastatic colorectal cancer proteins can be evaluated for diagnostic purposes or to screen candidate agents. In addition, the metastatic colorectal cancer nucleic acid sequences can be administered for gene therapy purposes, including the administration of antisense nucleic acids, or the metastatic colorectal cancer proteins (including antibodies and other modulators thereof) administered as therapeutic drugs or as protein or DNA vaccines.

Thus the present invention provides nucleic acid and protein sequences that are differentially expressed in metastatic colorectal cancer, herein termed “metastatic colorectal cancer sequences.” As outlined below, metastatic colorectal cancer sequences include those that are up-regulated (i.e., expressed at a higher level) in metastatic colorectal cancer, as well as those that are down-regulated (i.e., expressed at a lower level). In a preferred embodiment, the metastatic colorectal cancer sequences are from humans; however, as will be appreciated by those in the art, metastatic colorectal cancer sequences from other organisms may be useful in animal models of disease and drug evaluation; thus, other metastatic colorectal cancer sequences are provided, from vertebrates, including mammals, including rodents (rats, mice, hamsters, guinea pigs, etc.), primates, farm animals (including sheep, goats, pigs, cows, horses, etc.) and pets (dogs, cats, etc.). Metastatic colorectal cancer sequences from other organisms may be obtained using the techniques outlined below.

Metastatic colorectal cancer sequences can include both nucleic acid and amino acid sequences. As will be appreciated by those in the art and is more fully outlined below, metastatic colorectal cancer nucleic acid sequences are useful in a variety of applications, including diagnostic applications, which will detect naturally occurring nucleic acids, as well as screening applications; e.g., biochips comprising nucleic acid probes or PCR microtiter plates with selected probes to the metastatic colorectal cancer sequences can be generated.

A metastatic colorectal cancer sequence can be initially identified by substantial nucleic acid and/or amino acid sequence homology to the metastatic colorectal cancer sequences outlined herein. Such homology can be based upon the overall nucleic acid or amino acid sequence, and is generally determined as outlined below, using either homology programs or hybridization conditions.

For identifying metastatic colorectal cancer-associated sequences, the metastatic colorectal cancer screen typically includes comparing genes identified in different tissues, e.g., normal and cancerous tissues, or tumor tissue samples from patients who have metastatic disease vs. non metastatic tissue, or tumor tissue samples from patients who have been diagnosed with Dukes stage A or B cancer but have survived vs. metastatic tissue. Other suitable tissue comparisons include comparing metastatic colorectal cancer samples with metastatic cancer samples from other cancers, such as lung, breast, other gastrointestinal cancers, prostate, ovarian, etc. Samples of, e.g., Dukes stage B survivor tissue and tissue undergoing metastasis are applied to biochips comprising nucleic acid probes. The samples are first microdissected, if applicable, and treated as is known in the art for the preparation of mRNA. Suitable biochips are commercially available, e.g., from Affymetrix. Gene expression profiles as described herein are generated and the data analyzed.

In one embodiment, the genes showing changes in expression as between normal and disease states are compared to genes expressed in other normal tissues, preferably normal colon, but also including, and not limited to lung, heart, brain, liver, breast, kidney, muscle, prostate, small intestine, large intestine, spleen, bone and placenta. In a preferred embodiment, those genes identified during the metastatic colorectal cancer screen that are expressed in significant amounts in other tissues are removed from the profile, although in some embodiments, this is not necessary. That is, when screening for drugs, it is usually preferable that the target be disease specific, to minimize possible side effects.

In a preferred embodiment, metastatic colorectal cancer sequences are those that are up-regulated in metastatic colorectal cancer; that is, the expression of these genes is higher in the metastatic tissue as compared to non-metastatic cancerous tissue or normal colon tissue (see, e.g., Tables 1-26). “Up-regulation” as used herein means, when the ratio is presented as a number greater than one, that the ratio is greater than one, preferably 1.5 or greater, more preferably 2.0 or greater. All UniGene cluster identification numbers and accession numbers herein are for the GenBank sequence database and the sequences of the accession numbers are hereby expressly incorporated by reference. GenBank is known in the art, see, e.g., Benson, DA, et al., Nucleic Acids Research 26:1-7 (1998) and http://www.ncbi.nlm.nih.gov/. Sequences are also available in other databases, e.g., European Molecular Biology Laboratory (EMBL) and DNA Database of Japan (DDBJ).

In another preferred embodiment, metastatic colorectal cancer sequences are those that are down-regulated in the metastatic colorectal cancer; that is, the expression of these genes is lower in metastatic tissue as compared to non-metastatic cancerous tissue or normal colon tissue (see, e.g., Tables 1-26). “Down-regulation” as used herein means, when the ratio is presented as a number greater than one, that the ratio is greater than one, preferably 1.5 or greater, more preferably 2.0 or greater, or, when the ratio is presented as a number less than one, that the ratio is less than one, preferably 0.5 or less, more preferably 0.25 or less.

Informatics

The ability to identify genes that are over or under expressed in metastatic colorectal cancer can additionally provide high-resolution, high-sensitivity datasets which can be used in the areas of diagnostics, therapeutics, drug development, pharmacogenetics, protein structure, biosensor development, and other related areas. For example, the expression profiles can be used in diagnostic or prognostic evaluation of patients with metastatic colorectal cancer. Or as another example, subcellular toxicological information can be generated to better direct drug structure and activity correlation (see Anderson, Pharmaceutical Proteomics: Targets, Mechanism, and Function, paper presented at the IBC Proteomics conference, Coronado, Calif. (Jun. 11-12, 1998)). Subcellular toxicological information can also be utilized in a biological sensor device to predict the likely toxicological effect of chemical exposures and likely tolerable exposure thresholds (see U.S. Pat. No. 5,811,231). Similar advantages accrue from datasets relevant to other biomolecules and bioactive agents (e.g., nucleic acids, saccharides, lipids, drugs, and the like).

Thus, in another embodiment, the present invention provides a database that includes at least one set of assay data. The data contained in the database is acquired, e.g., using array analysis either singly or in a library format. The database can be in substantially any form in which data can be maintained and transmitted, but is preferably an electronic database. The electronic database of the invention can be maintained on any electronic device allowing for the storage of and access to the database, such as a personal computer, but is preferably distributed on a wide area network, such as the World Wide Web.

The focus of the present section on databases that include peptide sequence data is for clarity of illustration only. It will be apparent to those of skill in the art that similar databases can be assembled for assay data acquired using an assay of the invention.

The compositions and methods for identifying and/or quantitating the relative and/or absolute abundance of a variety of molecular and macromolecular species from a biological sample undergoing metastatic colorectal cancer, i.e., the identification of metastatic colorectal cancer-associated sequences described herein, provide an abundance of information, which can be correlated with pathological conditions, predisposition to disease, drug testing, therapeutic monitoring, gene-disease causal linkages, identification of correlates of immunity and physiological status, among others. Although the data generated from the assays of the invention is suited for manual review and analysis, in a preferred embodiment, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be viewed as a tree structure or as the merger of two or more such tree structures.

See also Mount et al., Bioinformatics (2001); Biological Sequence Analysis: Probabilistic Models of Proteins and Nucleic Acids (Durbin et al., eds., 1999); Bioinformatics: A Practical Guide to the Analysis of Genes and Proteins (Baxevanis & Oeullette eds., 1998)); Rashidi & Buehler, Bioinformatics: Basic Applications in Biological Science and Medicine (1999); Introduction to Computational Molecular Biology (Setubal et al., eds 1997); Bioinformatics: Methods and Protocols (Misener & Krawetz, eds, 2000); Bioinformatics: Sequence, Structure, and Databanks: A Practical Approach (Higgins & Taylor, eds., 2000); Brown, Bioinformatics: A Biologist's Guide to Biocomputing and the Internet (2001); Han & Kamber, Data Mining: Concepts and Techniques (2000); and Waterman, Introduction to Computational Biology: Maps, Sequences, and Genomes (1995).

The present invention provides a computer database comprising a computer and software for storing in computer-retrievable form assay data records cross-tabulated, e.g., with data specifying the source of the target-containing sample from which each sequence specificity record was obtained.

In an exemplary embodiment, at least one of the sources of target-containing sample is from a control tissue sample known to be free of pathological disorders. In a variation, at least one of the sources is a known pathological tissue specimen, e.g., a neoplastic lesion or another tissue specimen to be analyzed for metastatic colorectal cancer. In another variation, the assay records cross-tabulate one or more of the following parameters for each target species in a sample: (1) a unique identification code, which can include, e.g., a target molecular structure and/or characteristic separation coordinate (e.g., electrophoretic coordinates); (2) sample source; and (3) absolute and/or relative quantity of the target species present in the sample.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 target data records cross-tabulated with target source.

When the target is a peptide or nucleic acid, the invention preferably provides a method for identifying related peptide or nucleic acid sequences, comprising performing a computerized comparison between a peptide or nucleic acid sequence assay record stored in or retrieved from a computer storage device or database and at least one other sequence. The comparison can include a sequence analysis or comparison algorithm or computer program embodiment thereof (e.g., FASTA, TFASTA, GAP, BESTFIT) and/or the comparison may be of the relative amount of a peptide or nucleic acid sequence in a pool of sequences determined from a polypeptide or nucleic acid sample of a specimen.

The invention also preferably provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 10BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In a preferred embodiment, the invention provides a computer system for comparing a query target to a database containing an array of data structures, such as an assay result obtained by the method of the invention, and ranking database targets based on the degree of identity and gap weight to the target data. A central processor is preferably initialized to load and execute the computer program for alignment and/or comparison of the assay results. Data for a query target is entered into the central processor via an I/O device. Execution of the computer program results in the central processor retrieving the assay data from the data file, which comprises a binary description of an assay result.

The target data or record and the computer program can be transferred to secondary memory, which is typically random access memory (e.g., DRAM, SRAM, SGRAM, or SDRAM). Targets are ranked according to the degree of correspondence between a selected assay characteristic (e.g., binding to a selected affinity moiety) and the same characteristic of the query target and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

The invention also preferably provides the use of a computer system, such as that described above, which comprises: (1) a computer; (2) a stored bit pattern encoding a collection of peptide sequence specificity records obtained by the methods of the invention, which may be stored in the computer; (3) a comparison target, such as a query target; and (4) a program for alignment and comparison, typically with rank-ordering of comparison results on the basis of computed similarity values.

Characteristics of Metastatic Colorectal Cancer-Associated Proteins

Metastatic colorectal cancer proteins of the present invention may be classified as secreted proteins, transmembrane proteins or intracellular proteins. In one embodiment, the metastatic colorectal cancer protein is an intracellular protein. Intracellular proteins may be found in the cytoplasm and/or in the nucleus and/or in the organelles. Proteins containing one or more transmembrane domains that exclusively reside in organelles are also considered intracellular proteins. Intracellular proteins are involved in all aspects of cellular function and replication (including, e.g., signaling pathways); aberrant expression of such proteins often results in unregulated or disregulated cellular processes (see, e.g., Molecular Biology of the Cell (Alberts, ed., 3rd ed., 1994). For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterizing proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate. One useful database is Pfam (protein families), which is a large collection of multiple sequence alignments and hidden Markov models covering many common protein domains. Versions are available via the internet from Washington University in St. Louis, the Sanger Center in England, and the Karolinska Institute in Sweden (see, e.g., Bateman et al., Nuc. Acids Res. 28:263-266 (2000); Sonnhammer et al., Proteins 28:405-420 (1997); Bateman et al., Nuc. Acids Res. 27:260-262 (1999); and Sonnharnmer et al., Nuc. Acids Res. 26:320-322-(1998)).

In another embodiment, the metastatic colorectal cancer sequences are transmembrane proteins. Transmembrane proteins are molecules that span a phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels, pumps, and adenylyl cyclases contain numerous transmembrane domains. Many important cell surface receptors such as G protein coupled receptors (GPCRs) are classified as “seven transmembrane domain” proteins, as they contain 7 membrane spanning regions. Characteristics of transmembrane domains include approximately 20 consecutive hydrophobic amino acids that may be followed by charged amino acids. Therefore, upon analysis of the amino acid sequence of a particular protein, the localization and number of transmembrane domains within the protein may be predicted (see, e.g. PSORT web site http://psort.nibb.ac.jp/).

The extracellular domains of transmembrane proteins are diverse; however, conserved motifs are found repeatedly among various extracellular domains. Conserved structure and/or functions have been ascribed to different extracellular motifs. Many extracellular domains are involved in binding to other molecules. In one aspect, extracellular domains are found on receptors. Factors that bind the receptor domain include circulating ligands, which may be peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, hormones, neurotrophic factors and the like. Extracellular domains also bind to cell-associated molecules. In this respect, they mediate cell-cell interactions. Cell-associated ligands can be tethered to the cell, e.g., via a glycosylphosphatidylinositol (GPI) anchor, or may themselves be transmembrane proteins. Extracellular domains also associate with the extracellular matrix and contribute to the maintenance of the cell structure.

Metastatic colorectal cancer proteins that are transmembrane are particularly preferred in the present invention as they are readily accessible targets for extracellular immunotherapeutics, as are described herein. In addition, as outlined below, transmembrane proteins can be also useful in imaging modalities. Antibodies may be used to label such readily accessible proteins in situ or in histological analysis. Alternatively, antibodies can also label intracellular proteins, in which case analytical samples are typically permeablized to provide access to intracellular proteins.

It will also be appreciated by those in the art that a transmembrane protein can be made soluble by removing transmembrane sequences, e.g., through recombinant methods. Furthermore, transmembrane proteins that have been made soluble can be made to be secreted through recombinant means by adding an appropriate signal sequence.

In another embodiment, the metastatic colorectal cancer proteins are secreted proteins; the secretion of which can be either constitutive or regulated. These proteins have a signal peptide or signal sequence that targets the molecule to the secretory pathway. Secreted proteins are involved in numerous physiological events; by virtue of their circulating nature, they often serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. Metastatic colorectal cancer proteins that are secreted proteins are particularly preferred in the present invention as they serve as good targets for diagnostic markers, e.g., for blood, plasma, serum, or stool tests.

Use of Metastatic Colorectal Cancer Nucleic Acids

As described above, metastatic colorectal cancer sequence is initially identified by substantial nucleic acid and/or amino acid sequence homology or linkage to the metastatic colorectal cancer sequences outlined herein. Such homology can be based upon the overall nucleic acid or amino acid sequence, and is generally determined as outlined below, using either homology programs or hybridization conditions. Typically, linked sequences on a mRNA are found on the same molecule. The metastatic colorectal cancer nucleic acid sequences of the invention, e.g., the sequences in Tables 1-26, can be fragments of larger genes, i.e., they are nucleic acid segments. “Genes” in this context includes coding regions, non-coding regions, and mixtures of coding and non-coding regions. Accordingly, as will be appreciated by those in the art, using the sequences provided herein, extended sequences, in either direction, of the metastatic colorectal cancer genes can be obtained, using techniques well known in the art for cloning either longer sequences or the full length sequences; see Ausubel, et al., supra. Much can be done by informatics and many sequences can be clustered to include multiple sequences corresponding to a single gene, e.g., systems such as UniGene (see, http://www.ncbi.nlm.nih.gov/unigene/).

Once the metastatic colorectal cancer nucleic acid is identified, it can be cloned and, if necessary, its constituent parts recombined to form the entire metastatic colorectal cancer nucleic acid coding regions or the entire mRNA sequence. Once isolated from its natural source, e.g., contained within a plasmid or other vector or excised therefrom as a linear nucleic acid segment, the recombinant metastatic colorectal cancer nucleic acid can be further-used as a probe to identify and isolate other metastatic colorectal cancer nucleic acids, e.g., extended coding regions. It can also be used as a “precursor” nucleic acid to make modified or variant metastatic colorectal cancer nucleic acids and proteins.

The metastatic colorectal cancer nucleic acids of the present invention are used in several ways. In a first embodiment, nucleic acid probes to the metastatic colorectal cancer nucleic acids are made and attached to biochips to be used in screening and diagnostic methods, as outlined below, or for administration, e.g., for gene therapy, vaccine, and/or antisense applications. Alternatively, the metastatic colorectal cancer nucleic acids that include coding regions of metastatic colorectal cancer proteins can be put into expression vectors for the expression of metastatic colorectal cancer proteins, again for screening purposes or for administration to a patient.

In a preferred embodiment, nucleic acid probes to metastatic colorectal cancer nucleic acids (both the nucleic acid sequences outlined in the figures and/or the complements thereof) are made. The nucleic acid probes attached to the biochip are designed to be substantially complementary to the metastatic colorectal cancer nucleic acids, i.e. the target sequence (either the target sequence of the sample or to other probe sequences, e.g., in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under appropriate reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally complements of ORFs or whole genes are not used. In some embodiments, nucleic acids of lengths up to hundreds of bases can be used.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (i.e., have some sequence in common), or separate. In some cases, PCR primers may be used to amplify signal for higher sensitivity.

As will be appreciated by those in the art, nucleic acids can be attached or immobilized to a solid support in a wide variety of ways. By “immobilized” and grammatical equivalents herein is meant the association or binding between the nucleic acid probe and the solid support is sufficient to be stable under the conditions of binding, washing, analysis, and removal as outlined below. The binding can typically be covalent or non-covalent. By “non-covalent binding” and grammatical equivalents herein is typically meant one or more of electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to a biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant a material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, etc. In general, the substrates allow optical detection and do not appreciably fluoresce. A preferred substrate is described in copending application entitled Reusable Low Fluorescent Plastic Biochip, U.S. application Ser. No. 09/270,214, filed Mar. 15, 1999, herein incorporated by reference in its entirety.

Generally the substrate is planar, although as will be appreciated by those in the art, other configurations of substrates may be used as well. For example, the probes may be placed on the inside surface of a tube, for flow-through sample analysis to minimize sample volume. Similarly, the substrate may be flexible, such as a flexible foam, including closed cell foams made of particular plastics.

In a preferred embodiment, the surface of the biochip and the probe may be derivatized with chemical functional groups for subsequent attachment of the two. Thus, e.g., the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, e.g., using linkers as are known in the art; e.g., homo-or hetero-bifunctional linkers as are well known (see 1994 Pierce Chemical Company catalog, technical section on cross-linkers, pages 155-200). In addition, in some cases, additional linkers, such as alkyl groups (including substituted and heteroalkyl groups) may be used.

In this embodiment, oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In another embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the Affimetrix GeneChip™ technology.

Often, amplification-based assays are performed to measure the expression level of metastatic colorectal cancer-associated sequences. These assays are typically performed in conjunction with reverse transcription. In such assays, a metastatic colorectal cancer-associated nucleic acid sequence acts as a template in an amplification reaction (e.g., Polymerase Chain Reaction, or PCR). In a quantitative amplification, the amount of amplification product will be proportional to the amount of template in the original sample. Comparison to appropriate controls provides a measure of the amount of metastatic colorectal cancer-associated RNA. Methods of quantitative amplification are well known to those of skill in the art. Detailed protocols for quantitative PCR are provided, e.g., in Innis et al., PCR Protocols, A Guide to Methods and Applications (1990).

In some embodiments, a TaqMan based assay is used to measure expression. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, e.g., literature provided by Perkin-Elmer, e.g., www2.perkin-elmer.com).

Other suitable amplification methods include, but are not limited to, ligase chain reaction (LCR) (see Wu & Wallace, Genomics 4:560 (1989), Landegren et al., Science 241:1077 (1988), and Barringer et al., Gene 89:117 (1990)), transcription amplification (Kwoh et al., Proc. Natl. Acad. Sci. USA 86:1173 (1989)), self-sustained sequence replication (Guatelli et al., Proc. Nat. Acad. Sci. USA 87:1874 (1990)), dot PCR, and linker adapter PCR, etc.

Expression of Metastatic Colorectal Cancer Proteins From Nucleic Acids

In a preferred embodiment, metastatic colorectal cancer nucleic acids, e.g., encoding metastatic colorectal cancer proteins, are used to make a variety of expression vectors to express metastatic colorectal cancer proteins which can then be used in screening assays, as described below. Expression vectors and recombinant DNA technology are well known to those of skill in the art (see, e.g., Ausubel, supra, and Gene Expression Systems (Fernandez & Hoeffler, eds, 1999)) and are used to express proteins. The expression vectors may be either self-replicating extrachromosomal vectors or vectors which integrate into a host genome. Generally, these expression vectors include transcriptional and translational regulatory nucleic acid operably linked to the nucleic acid encoding the metastatic colorectal cancer protein. The term “control sequences” refers to DNA sequences used for the expression of an operably linked coding sequence in a particular host organism. Control sequences that are suitable for prokaryotes, e.g., include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is typically accomplished by ligation at convenient restriction sites. If such sites do not exist, synthetic oligonucleotide adaptors or linkers are used in accordance with conventional practice. Transcriptional and translational regulatory nucleic acid will generally be appropriate to the host cell used to express the metastatic colorectal cancer protein. Numerous types of appropriate expression vectors, and suitable regulatory sequences are known in the art for a variety of host cells.

In general, transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, an expression vector may comprise additional elements. For example, the expression vector may have two replication systems, thus allowing it to be maintained in two organisms, e.g., in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art (e.g., Fernandez & Hoeffler, supra).

In addition, in a preferred embodiment, the expression vector contains a selectable marker gene to allow the selection of transformed host cells. Selection genes are well known in the art and will vary with the host cell used.

The metastatic colorectal cancer proteins of the present invention are produced by culturing a host cell transformed with an expression vector containing nucleic acid encoding a metastatic colorectal cancer protein, under the appropriate conditions to induce or cause expression of the metastatic colorectal cancer protein. Conditions appropriate for metastatic colorectal cancer protein expression will vary with the choice of the expression vector and the host cell, and will be easily ascertained by one skilled in the art through routine experimentation or optimization. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect and animal cells, including mammalian cells. Of particular interest are Saccharomyces cerevisiae and other yeasts, E. coli, Bacillus subtilis, Sf9 cells, C129 cells, 293 cells, Neurospora, BHK, CHO, COS, HeLa cells, HLVEC (human umbilical vein endothelial cells), THP1 cells (a macrophage cell line) and various other human cells and cell lines.

In a preferred embodiment, the metastatic colorectal cancer proteins are expressed in mammalian cells. Mammalian expression systems are also known in the art, and include retroviral and adenoviral systems. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter (see, e.g., Fernandez & Hoeffler, supra). Typically, transcription termination and polyadenylation sequences recognized by mammalian cells are regulatory regions located 3′ to the translation stop codon and thus, together with the promoter elements, flank the coding sequence. Examples of transcription terminator and polyadenylation signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, metastatic colorectal cancer proteins are expressed in bacterial systems. Promoters from bacteriophage may also be used and are known in the art. In addition, synthetic promoters and hybrid promoters are also useful; e.g., the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the metastatic colorectal cancer protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others (e.g., Fernandez & Hoeffler, supra). The bacterial expression vectors are transformed into bacterial host cells using techniques well known in the art, such as calcium chloride treatment, electroporation, and others.

In one embodiment, metastatic colorectal cancer proteins are produced in insect cells. Expression vectors for the transformation of insect cells, and in particular, baculovirus-based expression vectors, are well known in the art.

In a preferred embodiment, metastatic colorectal cancer protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

The metastatic colorectal cancer protein may also be made as a fusion protein, using techniques well known in the art. Thus, e.g., for the creation of monoclonal antibodies, if the desired epitope is small, the metastatic colorectal cancer protein may be fused to a carrier protein to form an immunogen. Alternatively, the metastatic colorectal cancer protein may be made as a fusion protein to increase expression for affinity purification purposes, or for other reasons. For example, when the metastatic colorectal cancer protein is a metastatic colorectal cancer peptide, the nucleic acid encoding the peptide may be linked to other nucleic acid for expression purposes.

In a preferred embodiment, the metastatic colorectal cancer protein is purified or isolated after expression. Metastatic colorectal cancer proteins may be isolated or purified in a variety of appropriate ways. Standard purification methods include electrophoretic, molecular, immunological and chromatographic techniques, including ion exchange, hydrophobic, affinity, and reverse-phase HPLC chromatography, and chromatofocusing. For example, the metastatic colorectal cancer protein may be purified using a standard anti-metastatic colorectal cancer protein antibody column. Ultrafiltration and diafiltration techniques, in conjunction with protein concentration, are also useful. For general guidance in suitable purification techniques, see Scopes, Protein Purification (1982). The degree of purification necessary will vary depending on the use of the metastatic colorectal cancer protein. In some instances no purification will be necessary.

Once expressed and purified if necessary, the metastatic colorectal cancer proteins and nucleic acids are useful in a number of applications. They may be used as immunoselection reagents, as vaccine reagents, as screening agents, etc.

Variants of Metastatic Colorectal Cancer Proteins

In one embodiment, the metastatic colorectal cancer proteins are derivative or variant metastatic colorectal cancer proteins as compared to the wild-type sequence. That is, as outlined more fully below, the derivative metastatic colorectal cancer peptide will often contain at least one amino acid substitution, deletion or insertion, with amino acid substitutions being particularly preferred. The amino acid substitution, insertion or deletion may occur at a particular residue within the metastatic colorectal cancer peptide.

Also included within one embodiment of metastatic colorectal cancer proteins of the present invention are amino acid sequence variants. These variants typically fall into one or more of three classes: substitutional, insertional or deletional variants. These variants ordinarily are prepared by site specific mutagenesis of nucleotides in the DNA encoding the metastatic colorectal cancer protein, using cassette or PCR mutagenesis or other techniques, to produce DNA encoding the variant, and thereafter expressing the DNA in recombinant cell culture as outlined above. However, variant metastatic colorectal cancer protein fragments having up to about 100-150 residues may be prepared by in vitro synthesis. Amino acid sequence variants are characterized by the predetermined nature of the variation, a feature that sets them apart from naturally occurring allelic or interspecies variation of the metastatic colorectal cancer protein amino acid sequence. The variants typically exhibit the same qualitative biological activity as the naturally occurring analogue, although variants can also be selected which have modified characteristics as will be more fully outlined below.

While the site or region for introducing an amino acid sequence variation is often predetermined, the mutation per se need not be predetermined. For example, in order to optimize the performance of a mutation at a given site, random mutagenesis may be conducted at the target codon or region and the expressed metastatic colorectal cancer variants screened for the optimal combination of desired activity. Techniques exist for making substitution mutations at predetermined sites in DNA having a known sequence, e.g., M13 primer mutagenesis and PCR mutagenesis. Screening of the mutants is done using assays of metastatic colorectal cancer protein activities.

Amino acid substitutions are typically of single residues; insertions usually will be on the order of from about 1 to 20 amino acids, although considerably larger insertions may be occasionally tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. Larger changes may be tolerated in certain circumstances. When small alterations in the characteristics of a metastatic colorectal cancer protein are desired, substitutions are generally made in accordance with the amino acid substitution chart provided in the definition section.

Variants typically exhibit the same qualitative biological activity and will elicit the same immune response as the naturally-occurring analog, although variants also are selected to modify the characteristics of the metastatic colorectal cancer proteins as needed. Alternatively, the variant may be designed or reorganized such that the biological activity of the metastatic colorectal cancer protein is altered. For example, glycosylation sites may be altered or removed.

Covalent modifications of metastatic colorectal cancer polypeptides are included within the scope of this invention. One type of covalent modification includes reacting targeted amino acid residues of a metastatic colorectal cancer polypeptide with an organic derivatizing agent that is capable of reacting with selected side chains or the N-or C-terminal residues of a metastatic colorectal cancer polypeptide. Derivatization with bifunctional agents is useful, for instance, for crosslinking metastatic colorectal cancer polypeptides to a water-insoluble support matrix or surface for use in the method for purifying anti-metastatic colorectal cancer polypeptide antibodies or screening assays, as is more fully described below. Commonly used crosslinking agents include, e.g., 1,1-bis(diazoacetyl)-2-phenylethane, glutaraldehyde, N-hydroxysuccinimide esters, e.g., esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-((p-azidophenyl)dithio)propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the γ-amino groups of lysine, arginine, and histidine side chains (Creighton, Proteins: Structure and Molecular Properties, pp. 79-86 (1983)), acetylation of the N-terminal amine, and amidation of any C-terminal carboxyl group.

Another type of covalent modification of the metastatic colorectal cancer polypeptide encompassed by this invention is an altered native glycosylation pattern of the polypeptide. “Altering the native glycosylation pattern” is intended herein to mean adding to or deleting one or more carbohydrate moieties of a native sequence metastatic colorectal cancer polypeptide. Glycosylation patterns can be altered in many ways. For example the use of different cell types to express metastatic colorectal cancer-associated sequences can result in different glycosylation patterns.

Addition of glycosylation sites to metastatic colorectal cancer polypeptides may also be accomplished by altering the amino acid sequence thereof. The alteration may be made, e.g., by the addition of, or substitution by, one or more serine or threonine residues to the native sequence metastatic colorectal cancer polypeptide (for O-linked glycosylation sites). The metastatic colorectal cancer amino acid sequence may optionally be altered through changes at the DNA level, particularly by mutating the DNA encoding the metastatic colorectal cancer polypeptide at preselected bases such that codons are generated that will translate into the desired amino acids.

Another means of increasing the number of carbohydrate moieties on the metastatic colorectal cancer polypeptide is by chemical or enzymatic coupling of glycosides to the polypeptide. Such methods are described in the art, e.g., in WO 87/05330, and in Aplin & Wriston, CRC Crit. Rev. Biochem., pp. 259-306 (1981).

Removal of carbohydrate moieties present on the metastatic colorectal cancer polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo-and exo-glycosidases as described by Thotakura et al., Meth. Enzymol., 138:350 (1987).

Another type of covalent modification of metastatic colorectal cancer comprises linking the metastatic colorectal cancer polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Metastatic colorectal cancer polypeptides of the present invention may also be modified in a way to form chimeric molecules comprising a metastatic colorectal cancer polypeptide fused to another, heterologous polypeptide or amino acid sequence. In one embodiment, such a chimeric molecule comprises a fusion of a metastatic colorectal cancer polypeptide with a tag polypeptide which provides an epitope to which an anti-tag antibody can selectively bind. The epitope tag is generally placed at the amino-or carboxyl-terminus of the metastatic colorectal cancer polypeptide. The presence of such epitope-tagged forms of a metastatic colorectal cancer polypeptide can be detected using an antibody against the tag polypeptide. Also, provision of the epitope tag enables the metastatic colorectal cancer polypeptide to be readily purified by affinity purification using an anti-tag antibody or another type of affinity matrix that binds to the epitope tag. In an alternative embodiment, the chimeric molecule may comprise a fusion of a metastatic colorectal cancer polypeptide with an immunoglobulin or a particular region of an immunoglobulin. For a bivalent form of the chimeric molecule, such a fusion could be to the Fc region of an IgG molecule.

Various tag polypeptides and their respective antibodies are well known and examples include poly-histidine (poly-his) or poly-histidine-glycine (poly-his-gly) tags; HIS6 and metal chelation tags, the flu HA tag polypeptide and its antibody 12CA5 (Field et al., Mol. Cell. Biol. 8:2159-2165 (1988)); the c-myc tag and the 8F9, 3C7, 6E10, G4, B7 and 9E10 antibodies thereto (Evan et al., Molecular and Cellular Biology 5:3610-3616 (1985)); and the Herpes Simplex virus glycoprotein D (gD) tag and its antibody (Paborsky et al., Protein Engineering 3(6):547-553 (1990)). Other tag polypeptides include the Flag-peptide (Hopp et al., BioTechnology 6:1204-1210 (1988)); the KT3 epitope peptide (Martin et al., Science 255:192-194 (1992)); tubulin epitope peptide (Skinner et al., J. Biol. Chem. 266:15163-15166 (1991)); and the T7 gene 10 protein peptide tag (Lutz-Freyermuth et al., Proc. Natl. Acad. Sci. USA 87:6393-6397 (1990)).

Also included are other metastatic colorectal cancer proteins of the metastatic colorectal cancer family, and metastatic colorectal cancer proteins from other organisms, which are cloned and expressed as outlined below. Thus, probe or degenerate polymerase chain reaction (PCR) primer sequences may be used to find other related metastatic colorectal cancer proteins from primates or other organisms. As will be appreciated by those in the art, particularly useful probe and/or PCR primer sequences include unique areas of the metastatic colorectal cancer nucleic acid sequence. As is generally known in the art, preferred PCR primers are from about 15 to about 35 nucleotides in length, with from about 20 to about 30 being preferred, and may contain inosine as needed. PCR reaction conditions are well known in the art (e.g., Innis, PCR Protocols, supra).

Antibodies to Metastatic colorectal Cancer Proteins

In a preferred embodiment, when a metastatic colorectal cancer protein is to be used to generate antibodies, e.g., for immunotherapy or immunodiagnosis, the metastatic colorectal cancer protein should share at least one epitope or determinant with the full length protein. By “epitope” or “determinant” herein is typically meant a portion of a protein which will generate and/or bind an antibody or T-cell receptor in the context of MHC. Thus, in most instances, antibodies made to a smaller metastatic colorectal cancer protein will be able to bind to the full-length protein, particularly linear epitopes. In a preferred embodiment, the epitope is unique; that is, antibodies generated to a unique epitope show little or no cross-reactivity.

Methods of preparing polyclonal antibodies are well known (e.g., Coligan, supra; and Harlow & Lane, supra). Polyclonal antibodies can be raised in a mammal, e.g., by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of Tables 1-26 or fragment thereof or a fusion protein thereof. It may be useful to conjugate the immunizing agent to a protein known to be immunogenic in the mammal being immunized. Immunogenic proteins include, e.g., keyhole limpet hemocyanin, serum albumin, bovine thyroglobulin, and soybean trypsin inhibitor. Adjuvants include, e.g., Freund's complete adjuvant and MPL-TDM adjuvant (monophosphoryl Lipid A, synthetic trehalose dicorynomycolate). The immunization protocol may be selected by one skilled in the art.

The antibodies may, alternatively, be monoclonal antibodies. Monoclonal antibodies may be prepared using hybridoma methods, such as those described by Kohler & Milstein, Nature 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Tables 1-26, or fragment thereof, or a fusion protein thereof Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, pp. 59-103 (1986)). Immortalized cell lines are usually transformed mammalian cells, particularly myeloma cells of rodent, bovine and primate origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are typically monoclonal, preferably human or humanized, antibodies that have binding specificities for at least two different antigens or that have binding specificities for two epitopes on the same antigen. In one embodiment, one of the binding specificities is for a protein encoded by a nucleic acid of Tables 1-26 or a fragment thereof, the other one is for any other antigen, and preferably for a cell-surface protein or receptor or receptor subunit, preferably one that is tumor specific. Alternatively, tetramer-type technology may create multivalent reagents.

In a preferred embodiment, the antibodies to metastatic colorectal cancer protein are capable of reducing or eliminating a biological function of a metastatic colorectal cancer protein, as is described below. That is, the addition of anti-metastatic colorectal cancer protein antibodies (either polyclonal or preferably monoclonal) to metastatic colorectal cancer tissue (or cells containing metastatic colorectal cancer) may reduce or eliminate the metastatic colorectal cancer. Generally, at least a 25% decrease in activity, growth, size or the like is preferred, with at least about 50% being particularly preferred and about a 95-100% decrease being especially preferred.

In a preferred embodiment the antibodies to the metastatic colorectal cancer proteins are humanized antibodies (e.g., Xenerex Biosciences, Mederex, Inc., Abgenix, Inc., Protein Design Labs, Inc.) Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

Human-like antibodies can also be produced using various techniques known in the art, including phage display libraries (Hoogenboom & Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in virtually all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

By immunotherapy is meant treatment of metastatic colorectal cancer with an antibody raised against a metastatic colorectal cancer proteins. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. The antigen may be provided by injecting a polypeptide against which antibodies are desired to be raised into a recipient, or contacting the recipient with a nucleic acid capable of expressing the antigen and under conditions for expression of the antigen, leading to an immune response.

In a preferred embodiment the metastatic colorectal cancer proteins against which antibodies are raised are secreted proteins as described above. Without being bound by theory, antibodies used for treatment, bind and prevent the secreted protein from binding to its receptor, thereby inactivating the secreted metastatic colorectal cancer protein.

In another preferred embodiment, the metastatic colorectal cancer protein to which antibodies are raised is a transmembrane protein. Without being bound by theory, antibodies used for this treatment typically bind the extracellular domain of the metastatic colorectal cancer protein and prevent it from binding to other proteins, such as circulating ligands or cell-associated molecules. The antibody may cause down-regulation of the transmembrane metastatic colorectal cancer protein. The antibody may be a competitive, non-competitive or uncompetitive inhibitor of protein binding to the extracellular domain of the metastatic colorectal cancer protein. The antibody may be an antagonist of the metastatic colorectal cancer protein or may prevent activation of the transmembrane metastatic colorectal cancer protein. In some embodiments, when the antibody prevents the binding of other molecules to the metastatic colorectal cancer protein, the antibody prevents growth of the cell. The antibody may also be used to target or sensitize the cell to cytotoxic agents, including, but not limited to TNF-α, TNF-β, IL-1, INF-γ and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity or antigen-dependent cytotoxicity (ADCC). Thus, metastatic colorectal cancer is treated by administering to a patient antibodies directed against the transmembrane metastatic colorectal cancer protein. Antibody-labeling may activate a co-toxin, localize a toxin payload, or otherwise provide means to locally ablate cells.

In another preferred embodiment, the antibody is conjugated to an effector moiety. The effector moiety can be any number of molecules, including labeling moieties such as radioactive labels or fluorescent labels, or can be a therapeutic moiety. In one aspect the therapeutic moiety is a small molecule that modulates the activity of the metastatic colorectal cancer protein. In another aspect the therapeutic moiety modulates the activity of molecules associated with or in close proximity to the metastatic colorectal cancer protein. The therapeutic moiety may inhibit enzymatic activity such as protease or collagenase activity associated with metastatic colorectal cancer.

In a preferred embodiment, the therapeutic moiety can also be a cytotoxic agent. In this method, targeting the cytotoxic agent to metastatic colorectal cancer tissue or cells results in a reduction in the number of afflicted cells, thereby reducing symptoms associated with metastatic colorectal cancer. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against metastatic colorectal cancer proteins, or binding of a radionuclide to a chelating agent that has been covalently attached to the antibody. Targeting the therapeutic moiety to transmembrane metastatic colorectal cancer proteins not only serves to increase the local concentration of therapeutic moiety in the metastatic colorectal cancer afflicted area, but also serves to reduce deleterious side effects that may be associated with the therapeutic moiety.

In another preferred embodiment, the metastatic colorectal cancer protein against which the antibodies are raised is an intracellular protein. In this case, the antibody may be conjugated to a protein or other entity which facilitates entry into the cell. In one case, the antibody enters the cell by endocytosis. In another embodiment, a nucleic acid encoding the antibody is administered to the individual or cell. Moreover, wherein the metastatic colorectal cancer protein can be targeted within a cell, i.e., the nucleus, an antibody thereto contains a signal for that target localization, i.e., a nuclear localization signal.

The metastatic colorectal cancer antibodies of the invention specifically bind to metastatic colorectal cancer proteins. By “specifically bind” herein is meant that the antibodies bind to the protein with a K_{d} of at least about 0.1 mM, more usually at least about 1 μM, preferably at least about 0.1 μM or better, and most preferably, 0.01 μM or better. Selectivity of binding is also important.

Detection of Metastatic Colorectal Cancer Sequence for Diagnostic and Therapeutic Applications

In one aspect, the RNA expression levels of genes are determined for different cellular states in the metastatic colorectal cancer phenotype. Expression levels of genes in normal tissue (i.e., not undergoing metastatic colorectal cancer) and in metastatic colorectal cancer tissue (and in some cases, for varying severities of metastatic colorectal cancer that relate to prognosis, as outlined below) are evaluated to provide expression profiles. An expression profile of a particular cell state or point of development is essentially a “fingerprint” of the state. While two states may have any particular gene similarly expressed, the evaluation of a number of genes simultaneously allows the generation of a gene expression profile that is reflective of the state of the cell. By comparing expression profiles of cells in different states, information regarding which genes are important (including both up- and down-regulation of genes) in each of these states is obtained. Then, diagnosis may be performed or confirmed to determine whether a tissue sample has the gene expression profile of normal or cancerous tissue. This will provide for molecular diagnosis of related conditions.

“Differential expression,” or grammatical equivalents as used herein, refers to qualitative or quantitative differences in the temporal and/or cellular gene expression patterns within and among cells and tissue. Thus, a differentially expressed gene can qualitatively have its expression altered, including an activation or inactivation, in, e.g., normal versus metastatic colorectal cancer tissue. Genes may be turned on or turned off in a particular state, relative to another state thus permitting comparison of two or more states. A qualitatively regulated gene will exhibit an expression pattern within a state or cell type which is detectable by standard techniques. Some genes will be expressed in one state or cell type, but not in both. Alternatively, the difference in expression may be quantitative, e.g., in that expression is increased or decreased; i.e., gene expression is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays, Lockhart, Nature Biotechnology 14:1675-1680 (1996), hereby expressly incorporated by reference. Other techniques include, but are not limited to, quantitative reverse transcriptase PCR, northern analysis and RNase protection. As outlined above, preferably the change in expression (i.e., upregulation or downregulation) is typically at least about 50%, more preferably at least about 100%, more preferably at least about 150%, more preferably at least about 200%, with from 300 to at least 1000% being especially preferred.

Evaluation may be at the gene transcript, or the protein level. The amount of gene expression may be monitored using nucleic acid probes to the DNA or RNA equivalent of the gene transcript, and the quantification of gene expression levels, or, alternatively, the final gene product itself (protein) can be monitored, e.g., with antibodies to the metastatic colorectal cancer protein and standard immunoassays (ELISAs, etc.) or other techniques, including mass spectroscopy assays, 2D gel electrophoresis assays, etc. Proteins corresponding to metastatic colorectal cancer genes, i.e., those identified as being important in a metastatic colorectal cancer phenotype, can be evaluated in a metastatic colorectal cancer diagnostic test.

In a preferred embodiment, gene expression monitoring is performed simultaneously on a number of genes.

The metastatic colorectal cancer nucleic acid probes may be attached to biochips as outlined herein for the detection and quantification of metastatic colorectal cancer sequences in a particular cell. The assays are further described below in the example. PCR techniques can be used to provide greater sensitivity. Multiple protein expression monitoring can be performed as well. Similarly, these assays may be performed on an individual basis as well.

In a preferred embodiment nucleic acids encoding the metastatic colorectal cancer protein are detected. Although DNA or RNA encoding the metastatic colorectal cancer protein may be detected, of particular interest are methods wherein an mRNA encoding a metastatic colorectal cancer protein is detected. Probes to detect mRNA can be a nucleotide/deoxynucleotide probe that is complementary to and hybridizes with the mRNA and includes, but is not limited to, oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding a metastatic colorectal cancer protein is detected by binding the digoxygenin with an anti-digoxygenin secondary antibody and developed with nitro blue tetrazolium and 5-bromo-4-chloro-3-indoyl phosphate.

In a preferred embodiment, various proteins from the three classes of proteins as described herein (secreted, transmembrane or intracellular proteins) are used in diagnostic assays. The metastatic colorectal cancer proteins, antibodies, nucleic acids, modified proteins and cells containing metastatic colorectal cancer sequences are used in diagnostic assays. This can be performed on an individual gene or corresponding polypeptide level. In a preferred embodiment, the expression profiles are used, preferably in conjunction with high throughput screening techniques to allow monitoring for expression profile genes and/or corresponding polypeptides.

As described and defined herein, metastatic colorectal cancer proteins, including intracellular, transmembrane or secreted proteins, find use as markers of metastatic colorectal cancer. Detection of these proteins in putative metastatic colorectal cancer tissue allows for detection or diagnosis of metastatic colorectal cancer. In one embodiment, antibodies are used to detect metastatic colorectal cancer proteins. A preferred method separates proteins from a sample by electrophoresis on a gel (typically a denaturing and reducing protein gel, but may be another type of gel, including isoelectric focusing gels and the like). Following separation of proteins, the metastatic colorectal cancer protein is detected, e.g., by immunoblotting with antibodies raised against the metastatic colorectal cancer protein. Methods of immunoblotting are well known to those of ordinary skill in the art.

In another preferred method, antibodies to the metastatic colorectal cancer protein find use in in situ imaging techniques, e.g., in histology (e.g., Methods in Cell Biology: Antibodies in Cell Biology, volume 37 (Asai, ed. 1993)). In this method cells are contacted with from one to many antibodies to the metastatic colorectal cancer protein(s). Following washing to remove non-specific antibody binding, the presence of the antibody or antibodies is detected. In one embodiment the antibody is detected by incubating with a secondary antibody that contains a detectable label, e.g., multicolor fluorescence or confocal imaging. In another method the primary antibody to the metastatic colorectal cancer protein(s) contains a detectable label, e.g., an enzyme marker that can act on a substrate. In another preferred embodiment each one of multiple primary antibodies contains a distinct and detectable label. This method finds particular use in simultaneous screening for a plurality of metastatic colorectal cancer proteins. Many other histological imaging techniques are also provided by the invention.

In a preferred embodiment the label is detected in a fluorometer which has the ability to detect and distinguish emissions of different wavelengths. In addition, a fluorescence activated cell sorter (FACS) can be used in the method.

In another preferred embodiment, antibodies find use in diagnosing metastatic colorectal cancer from blood, serum, plasma, stool, and other samples. Such samples, therefore, are useful as samples to be probed or tested for the presence of metastatic colorectal cancer proteins. Antibodies can be used to detect a metastatic colorectal cancer protein by previously described immunoassay techniques including ELISA, immunoblotting (western blotting), immunoprecipitation, BIACORE technology and the like. Conversely, the presence of antibodies may indicate an immune response against an endogenous metastatic colorectal cancer protein or vaccine.

In a preferred embodiment, in situ hybridization of labeled metastatic colorectal cancer nucleic acid probes to tissue arrays is done. For example, arrays of tissue samples, including metastatic colorectal cancer tissue and/or normal tissue, are made. In situ hybridization (see, e.g., Ausubel, supra) is then performed. When comparing the fingerprints between an individual and a standard, the skilled artisan can make a diagnosis, a prognosis, or a prediction based on the findings. It is further understood that the genes which indicate the diagnosis may differ from those which indicate the prognosis and molecular profiling of the condition of the cells may lead to distinctions between responsive or refractory conditions or may be predictive of outcomes.

In a preferred embodiment, the metastatic colorectal cancer proteins, antibodies, nucleic acids, modified proteins and cells containing metastatic colorectal cancer sequences are used in prognosis assays. As above, gene expression profiles can be generated that correlate to metastatic colorectal cancer, in terms of long term prognosis. Again, this may be done on either a protein or gene level, with the use of genes being preferred. As above, metastatic colorectal cancer probes may be attached to biochips for the detection and quantification of metastatic colorectal cancer sequences in a tissue or patient. The assays proceed as outlined above for diagnosis. PCR method may provide more sensitive and accurate quantification.

Assays for Therapeutic Compounds

In a preferred embodiment members of the three classes of proteins as described herein are used in drug screening assays. The metastatic colorectal cancer proteins, antibodies, nucleic acids, modified proteins and cells containing metastatic colorectal cancer sequences are used in drug screening assays or by evaluating the effect of drug candidates on a “gene expression profile” or expression profile of polypeptides. In a preferred embodiment, the expression profiles are used, preferably in conjunction with high throughput screening techniques to allow monitoring for expression profile genes after treatment with a candidate agent (e.g., Zlokarnik, et al., Science 279:84-8 (1998); Heid, Genome Res 6:986-94, 1996).

In a preferred embodiment, the metastatic colorectal cancer proteins, antibodies, nucleic acids, modified proteins and cells containing the native or modified metastatic colorectal cancer proteins are used in screening assays. That is, the present invention provides novel methods for screening for compositions which modulate the metastatic colorectal cancer phenotype or an identified physiological function of a metastatic colorectal cancer protein. As above, this can be done on an individual gene level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the differentially expressed genes herein, a variety of assays may be applied. In a preferred embodiment, assays may be run on an individual gene or protein level. That is, having identified a particular gene with altered regulation in metastatic colorectal cancer, test compounds can be screened for the ability to modulate gene expression or for binding to the metastatic colorectal cancer protein. “Modulation” thus includes an increase or a decrease in gene expression. The preferred amount of modulation will depend on the original change of the gene expression in normal versus tissue undergoing metastatic colorectal cancer, with changes of at least 10%, preferably 50%, more preferably 100-300%, and in some embodiments 300-1000% or greater. Thus, if a gene exhibits a 4-fold increase in metastatic colorectal cancer tissue compared to normal tissue, a decrease of about four-fold is often desired; similarly, a 10-fold decrease in metastatic colorectal cancer tissue compared to normal tissue often provides a target value of a 10-fold increase in expression to be induced by the test compound.

The amount of gene expression may be monitored using nucleic acid probes and the quantification of gene expression levels, or, alternatively, the gene product itself can be monitored, e.g., through the use of antibodies to the metastatic colorectal cancer protein and standard immunoassays. Proteomics and separation techniques may also allow quantification of expression.

In a preferred embodiment, gene or protein expression monitoring of a number of entities, i.e., an expression profile, is monitored simultaneously. Such profiles will typically involve a plurality of those entities described herein.

In this embodiment, the metastatic colorectal cancer nucleic acid probes are attached to biochips as outlined herein for the detection and quantification of metastatic colorectal cancer sequences in a particular cell. Alternatively, PCR may be used. Thus, a series, e.g., of microtiter plate, may be used with dispensed primers in desired wells. A PCR reaction can then be performed and analyzed for each well.

Expression monitoring can be performed to identify compounds that modify the expression of one or more metastatic colorectal cancer-associated sequences, e.g., a polynucleotide sequence set out in Tables 1-26. Generally, in a preferred embodiment, a test compound is added to the cells prior to analysis. Moreover, screens are also provided to identify agents that modulate metastatic colorectal cancer, modulate metastatic colorectal cancer proteins, bind to a metastatic colorectal cancer protein, or interfere with the binding of a metastatic colorectal cancer protein and an antibody, substrate, or other binding partner.

The term “test compound” or “drug candidate” or “modulator” or grammatical equivalents as used herein describes any molecule, e.g., protein, oligopeptide, small organic molecule, polysaccharide, polynucleotide, etc., to be tested for the capacity to directly or indirectly alter the metastatic colorectal cancer phenotype or the expression of a metastatic colorectal cancer sequence, e.g., a nucleic acid or protein sequence. In preferred embodiments, modulators alter expression profiles of nucleic acids or proteins provided herein. In one embodiment, the modulator suppresses a metastatic colorectal cancer phenotype, e.g., to a normal tissue fingerprint. In another embodiment, a modulator induces a metastatic colorectal cancer phenotype. Generally, a plurality of assay mixtures are run in parallel with different agent concentrations to obtain a differential response to the various concentrations. Typically, one of these concentrations serves as a negative control, i.e., at zero concentration or below the level of detection.

In one aspect, a modulator will neutralize the effect of a metastatic colorectal cancer protein. By “neutralize” is meant that activity of a protein and the consequent effect on the cell is inhibited or blocked.

In certain embodiments, combinatorial libraries of potential modulators will be screened for an ability to bind to a metastatic colorectal cancer polypeptide or to modulate activity. Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et al., J. Med. Chem. 37(9):1233-1251 (1994)).

Preparation and screening of combinatorial chemical libraries is well known to those of skill in the art. Such combinatorial chemical libraries include, but are not limited to, peptide libraries (see, e.g., U.S. Pat. No. 5,010,175, Furka, Pept. Prot. Res. 37:487-493 (1991), Houghton et al., Nature, 354:84-88 (1991)), peptoids (PCT Publication No WO 91/19735), encoded peptides (PCT Publication WO 93/20242), random bio-oligomers (PCT Publication WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with a Beta-D-Glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho, et al., Science 261:1303 (1993)), and/or peptidyl phosphonates (Campbell et al., J. Org. Chem. 59:658 (1994)). See, generally, Gordon et al., J. Med. Chem. 37:1385 (1994), nucleic acid libraries (see, e.g., Strategene, Corp.), peptide nucleic acid libraries (see, e.g., U.S. Pat. No. 5,539,083), antibody libraries (see, e.g., Vaughn et al., Nature Biotechnology 14(3):309-314 (1996), and PCT/US96/10287), carbohydrate libraries (see, e.g., Liang et al., Science 274:1520-1522 (1996), and U.S. Pat. No. 5,593,853), and small organic molecule libraries (see, e.g., benzodiazepines, Baum, C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514; and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. The above devices, with appropriate modification, are suitable for use with the present invention. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

The assays to identify modulators are amenable to high throughput screening. Preferred assays thus detect modulation of metastatic colorectal cancer gene transcription, polypeptide expression, and polypeptide activity.

High throughput assays for evaluating the presence, absence, quantification, or other properties of particular nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate procedures, including sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

In one embodiment, modulators are proteins, often naturally occurring proteins or fragments of naturally occurring proteins. Thus, e.g., cellular extracts containing proteins, or random or directed digests of proteinaceous cellular extracts, may be used. In this way libraries of proteins may be made for screening in the methods of the invention. Particularly preferred in this embodiment are libraries of bacterial, fungal, viral, and mammalian proteins, with the latter being preferred, and human proteins being especially preferred. Particularly useful test compound will be directed to the class of proteins to which the target belongs, e.g., substrates for enzymes or ligands and receptors.

In a preferred embodiment, modulators are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that the nucleic acid or peptide consists of essentially random sequences of nucleotides and amino acids, respectively. Since these random peptides (or nucleic acids, discussed below) are often chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. In a preferred embodiment, the nucleotides or amino acid residues are randomized within a defined class, e.g., of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc.

Modulators of metastatic colorectal cancer can also be nucleic acids, as defined above.

As described above generally for proteins, nucleic acid modulating agents may be naturally occurring nucleic acids, random nucleic acids, or “biased” random nucleic acids. Digests of procaryotic or eucaryotic genomes may be used as is outlined above for proteins.

In a preferred embodiment, the candidate compounds are organic chemical moieties, a wide variety of which are available in the literature.

After a candidate agent has been added and the cells allowed to incubate for some period of time, the sample containing a target sequence is analyzed. If required, the target sequence is prepared using known techniques. For example, the sample may be treated to lyse the cells, using known lysis buffers, electroporation, etc., with purification and/or amplification such as PCR performed as appropriate. For example, an in vitro transcription with labels covalently attached to the nucleotides is performed. Generally, the nucleic acids are labeled with biotin-FITC or PE, or with cy3 or cy5.

In a preferred embodiment, the target sequence is labeled with, e.g., a fluorescent, a chemiluminescent, a chemical, or a radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. Unbound labeled streptavidin is typically removed prior to analysis.

Nucleic acid assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions which allow formation of the label probe hybridization complex only in the presence of target. Stringency can be controlled by altering a step parameter that is a thermodynamic variable, including, but not limited to, temperature, formamide concentration, salt concentration, chaotropic salt concentration, pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways. Components of the reaction may be added simultaneously, or sequentially, in different orders, with preferred embodiments outlined below. In addition, the reaction may include a variety of other reagents. These include salts, buffers, neutral proteins, e.g., albumin, detergents, etc. which may be used to facilitate optimal hybridization and detection, and/or reduce non-specific or background interactions. Reagents that otherwise improve the efficiency of the assay, such as protease inhibitors, nuclease inhibitors, anti-microbial agents, etc., may also be used as appropriate, depending on the sample preparation methods and purity of the target.

The assay data are analyzed to determine the expression levels, and changes in expression levels as between states, of individual genes, forming a gene expression profile.

Screens are performed to identify modulators of the metastatic colorectal cancer phenotype. In one embodiment, screening is performed to identify modulators that can induce or suppress a particular expression profile, thus preferably generating the associated phenotype. In another embodiment, e.g., for diagnostic applications, having identified differentially expressed genes important in a particular state, screens can be performed to identify modulators that alter expression of individual genes. In an another embodiment, screening is performed to identify modulators that alter a biological function of the expression product of a differentially expressed gene. Again, having identified the importance of a gene in a particular state, screens are performed to identify agents that bind and/or modulate the biological activity of the gene product, or evaluate genetic polymorphisms.

Genes can be screened for those that are induced in response to a candidate agent. After identifying a modulator based upon its ability to suppress a metastatic colorectal cancer expression pattern leading to a normal expression pattern, or to modulate a single metastatic colorectal cancer gene expression profile so as to mimic the expression of the gene from normal tissue, a screen as described above can be performed to identify genes that are specifically modulated in response to the agent. Comparing expression profiles between normal tissue and agent treated metastatic colorectal cancer tissue reveals genes that are not expressed in normal tissue or metastatic colorectal cancer tissue, but are expressed in agent treated tissue. These agent-specific sequences can be identified and used by methods described herein for metastatic colorectal cancer genes or proteins. In particular these sequences and the proteins they encode find use in marking or identifying agent treated cells. In addition, antibodies can be raised against the agent induced proteins and used to target novel therapeutics to the treated metastatic colorectal cancer tissue sample.

Thus, in one embodiment, a test compound is administered to a population of metastatic colorectal cancer cells, that have an associated metastatic colorectal cancer expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e., a peptide) may be put into a viral construct such as an adenoviral or retroviral construct, and added to the cell, such that expression of the peptide agent is accomplished, e.g., PCT US97/01019. Regulatable gene therapy systems can also be used.

Once the test compound has been administered to the cells, the cells can be washed if desired and are allowed to incubate under preferably physiological conditions for some period of time. The cells are then harvested and a new gene expression profile is generated, as outlined herein.

Thus, e.g., metastatic colorectal cancer tissue may be screened for agents that modulate, e.g., induce or suppress the metastatic colorectal cancer phenotype. A change in at least one gene, preferably many, of the expression profile indicates that the agent has an effect on metastatic colorectal cancer activity. By defining such a signature for the metastatic colorectal cancer phenotype, screens for new drugs that alter the phenotype can be devised. With this approach, the drug target need not be known and need not be represented in the original expression screening platform, nor does the level of transcript for the target protein need to change.

Measure of metastatic colorectal cancer polypeptide activity, or of metastatic colorectal cancer or the metastatic colorectal cancer phenotype can be performed using a variety of assays. For example, the effects of the test compounds upon the function of the metastatic polypeptides can be measured by examining parameters described above. A suitable physiological change that affects activity can be used to assess the influence of a test compound on the polypeptides of this invention. When the functional consequences are determined using intact cells or animals, one can also measure a variety of effects such as, in the case of metastatic colorectal cancer associated with tumors, tumor growth, tumor metastasis, neovascularization, hormone release, transcriptional changes to both known and uncharacterized genetic markers (e.g., northern blots), changes in cell metabolism such as cell growth or pH changes, and changes in intracellular second messengers such as cGMP. In the assays of the invention, mammalian metastatic colorectal cancer polypeptide is typically used, e.g., mouse, preferably human.

Assays to identify compounds with modulating activity can be performed in vitro. For example, a colorectal cancer polypeptide is first contacted with a potential modulator and incubated for a suitable amount of time, e.g., from 0.5 to 48 hours. In one embodiment, the metastatic colorectal cancer polypeptide levels are determined in vitro by measuring the level of protein or mRNA. The level of protein is measured using immunoassays such as western blotting, ELISA and the like with an antibody that selectively binds to the metastatic colorectal cancer polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, a reporter gene system can be devised using the metastatic colorectal cancer protein promoter operably linked to a reporter gene such as luciferase, green fluorescent protein, CAT, or β-gal. The reporter construct is typically transfected into a cell. After treatment with a potential modulator, the amount of reporter gene transcription, translation, or activity is measured according to standard techniques known to those of skill in the art.

In a preferred embodiment, as outlined above, screens may be done on individual genes and gene products (proteins). That is, having identified a particular differentially expressed gene as important in a particular state, screening of modulators of the expression of the gene or the gene product itself can be done. The gene products of differentially expressed genes are sometimes referred to herein as “metastatic colorectal cancer proteins.” The metastatic colorectal cancer protein may be a fragment, or alternatively, be the full length protein to a fragment shown herein.

In one embodiment, screening for modulators of expression of specific genes is performed. Typically, the expression of only one or a few genes are evaluated. In another embodiment, screens are designed to first find compounds that bind to differentially expressed proteins. These compounds are then evaluated for the ability to modulate differentially expressed activity. Moreover, once initial candidate compounds are identified, variants can be further screened to better evaluate structure activity relationships.

In a preferred embodiment, binding assays are done. In general, purified or isolated gene product is used; that is, the gene products of one or more differentially expressed nucleic acids are made. For example, antibodies are generated to the protein gene products, and standard immunoassays are run to determine the amount of protein present. Alternatively, cells comprising the metastatic colorectal cancer proteins can be used in the assays.

Thus, in a preferred embodiment, the methods comprise combining a metastatic colorectal cancer protein and a candidate compound, and determining the binding of the compound to the metastatic colorectal cancer protein. Preferred embodiments utilize the human metastatic colorectal cancer protein, although other mammalian proteins may also be used, e.g., for the development of animal models of human disease. In some embodiments, as outlined herein, variant or derivative metastatic colorectal cancer proteins may be used.

Generally, in a preferred embodiment of the methods herein, the metastatic colorectal cancer protein or the candidate agent is non-diffusably bound to an insoluble support having isolated sample receiving areas (e.g., a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, teflon™, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the metastatic colorectal cancer protein is bound to the support, and a test compound is added to the assay. Alternatively, the candidate agent is bound to the support and the metastatic colorectal cancer protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the test modulating compound to the metastatic colorectal cancer protein may be done in a number of ways. In a preferred embodiment, the compound is labeled, and binding determined directly, e.g., by attaching all or a portion of the metastatic colorectal cancer protein to a solid support, adding a labeled candidate agent (e.g., a fluorescent label), washing off excess reagent, and determining whether the label is present on the solid support. Various blocking and washing steps may be utilized as appropriate.

In some embodiments, only one of the components is labeled, e.g., the proteins (or proteinaceous candidate compounds) can be labeled. Alternatively, more than one component can be labeled with different labels, e.g., ¹²⁵I for the proteins and a fluorophor for the compound. Proximity reagents, e.g., quenching or energy transfer reagents are also useful.

In one embodiment, the binding of the test compound is determined by competitive binding assay. The competitor is a binding moiety known to bind to the target molecule (i.e., a metastatic colorectal cancer protein), such as an antibody, peptide, binding partner, ligand, etc. Under certain circumstances, there may be competitive binding between the compound and the binding moiety, with the binding moiety displacing the compound. In one embodiment, the test compound is labeled. Either the compound, or the competitor, or both, is added first to the protein for a time sufficient to allow binding, if present. Incubations may be performed at a temperature which facilitates optimal activity, typically between 4 and 40° C. Incubation periods are typically optimized, e.g., to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the test compound. Displacement of the competitor is an indication that the test compound is binding to the metastatic colorectal cancer protein and thus is capable of binding to, and potentially modulating, the activity of the metastatic colorectal cancer protein. In this embodiment, either component can be labeled. Thus, e.g., if the competitor is labeled, the presence of label in the wash solution indicates displacement by the agent. Alternatively, if the test compound is labeled, the presence of the label on the support indicates displacement.

In an alternative embodiment, the test compound is added first, with incubation and washing, followed by the competitor. The absence of binding by the competitor may indicate that the test compound is bound to the metastatic colorectal cancer protein with a higher affinity. Thus, if the test compound is labeled, the presence of the label on the support, coupled with a lack of competitor binding, may indicate that the test compound is capable of binding to the metastatic colorectal cancer protein.

In a preferred embodiment, the methods comprise differential screening to identity agents that are capable of modulating the activity of the metastatic colorectal cancer proteins. In this embodiment, the methods comprise combining a metastatic colorectal cancer protein and a competitor in a first sample. A second sample comprises a test compound, a metastatic colorectal cancer protein, and a competitor. The binding of the competitor is determined for both samples, and a change, or difference in binding between the two samples indicates the presence of an agent capable of binding to the metastatic colorectal cancer protein and potentially modulating its activity. That is, if the binding of the competitor is different in the second sample relative to the first sample, the agent is capable of binding to the metastatic colorectal cancer protein.

Alternatively, differential screening is used to identify drug candidates that bind to the native metastatic colorectal cancer protein, but cannot bind to modified metastatic colorectal cancer proteins. The structure of the metastatic colorectal cancer protein may be modeled, and used in rational drug design to synthesize agents that interact with that site. Drug candidates that affect the activity of a metastatic colorectal cancer protein are also identified by screening drugs for the ability to either enhance or reduce the activity of the protein.

Positive controls and negative controls may be used in the assays. Preferably control and test samples are performed in at least triplicate to obtain statistically significant results. Incubation of all samples is for a time sufficient for the binding of the agent to the protein. Following incubation, samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g., albumin, detergents, etc. which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in an order that provides for the requisite binding.

In a preferred embodiment, the invention provides methods for screening for a compound capable of modulating the activity of a metastatic colorectal cancer protein. The methods comprise adding a test compound, as defined above, to a cell comprising metastatic colorectal cancer proteins. Preferred cell types include almost any cell. The cells contain a recombinant nucleic acid that encodes a metastatic colorectal cancer protein. In a preferred embodiment, a library of candidate agents are tested on a plurality of cells.

In one aspect, the assays are evaluated in the presence or absence or previous or subsequent exposure of physiological signals, e.g., hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, compounds that modulate metastatic colorectal cancer agents are identified. Compounds with pharmacological activity are able to enhance or interfere with the activity of the metastatic colorectal cancer protein. Once identified, similar structures are evaluated to identify critical structural feature of the compound.

In one embodiment, a method of inhibiting metastatic colorectal cancer cell division is provided. The method comprises administration of a metastatic colorectal cancer inhibitor. In another embodiment, a method of inhibiting metastatic colorectal cancer is provided. The method comprises administration of a metastatic colorectal cancer inhibitor. In a further embodiment, methods of treating cells or individuals with metastatic colorectal cancer are provided. The method comprises administration of a metastatic colorectal cancer inhibitor.

A variety of cell growth, proliferation, and metastasis assays are known to those of skill in the art, as described below.

Soft Agar Growth or Colony Formation in Suspension

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumor suppressor genes, regenerate normal phenotype and require a solid substrate to attach and grow. Soft agar growth or colony formation in suspension assays can be used to identify modulators of metastatic colorectal cancer sequences, which when expressed in host cells, inhibit abnormal cellular proliferation and transformation. A therapeutic compound would reduce or eliminate the host cells' ability to grow in stirred suspension culture or suspended in semi-solid media, such as semi-solid or soft.

Techniques for soft agar growth or colony formation in suspension assays are described in Freshney, Culture of Animal Cells a Manual of Basic Technique (3^{rd} ed., 1994), herein incorporated by reference. See also, the methods section of Garkavtsev et al. (1996), supra, herein incorporated by reference.

Contact Inhibition and Density Limitation of Growth

Normal cells typically grow in a flat and organized pattern in a petri dish until they touch other cells. When the cells touch one another, they are contact inhibited and stop growing. When cells are transformed, however, the cells are not contact inhibited and continue to grow to high densities in disorganized foci. Thus, the transformed cells grow to a higher saturation density than normal cells. This can be detected morphologically by the formation of a disoriented monolayer of cells or rounded cells in foci within the regular pattern of normal surrounding cells. Alternatively, labeling index with (³H)-thymidine at saturation density can be used to measure density limitation of growth. See Freshney (1994), supra. The transformed cells, when transfected with tumor suppressor genes, regenerate a normal phenotype and become contact inhibited and would grow to a lower density.

In this assay, labeling index with (³H)-thymidine at saturation density is a preferred method of measuring density limitation of growth. Transformed host cells are transfected with a metastatic colorectal cancer-associated sequence and are grown for 24 hours at saturation density in non-limiting medium conditions. The percentage of cells labeling with (³H)-thymidine is determined autoradiographically. See, Freshney (1994), supra.

Growth Factor or Serum Dependence

Transformed cells have a lower serum dependence than their normal counterparts (see, e.g., Temin, J. Natl. Cancer Insti. 37:167-175 (1966); Eagle et al., J. Exp. Med. 131:836-879 (1970)); Freshney, supra. This is in part due to release of various growth factors by the transformed cells. Growth factor or serum dependence of transformed host cells can be compared with that of control.

Tumor Specific Markers Levels

Tumor cells release an increased amount of certain factors (hereinafter “tumor specific markers”) than their normal counterparts. For example, plasminogen activator (PA) is released from human glioma at a higher level than from normal brain cells (see, e.g., Gullino, Angiogenesis, tumor vascularization, and potential interference with tumor growth. in Biological Responses in Cancer, pp. 178-184 (Mihich (ed.) 1985)). Similarly, Tumor angiogenesis factor (TAF) is released at a higher level in tumor cells than their normal counterparts. See, e.g., Folkman, Angiogenesis and Cancer, Sem Cancer Biol. (1992)).

Various techniques which measure the release of these factors are described in Freshney (1994), supra. Also, see, Unkless et al., J. Biol. Chem. 249:4295-4305 (1974); Strickland & Beers, J. Biol. Chem. 251:5694-5702 (1976); Whur et al., Br. J. Cancer 42:305-312 (1980); Gullino, Angiogenesis, tumor vascularization, and potential interference with tumor growth. in Biological Responses in Cancer, pp. 178-184 (Mihich (ed.) 1985); Freshney Anticancer Res. 5:111-130 (1985).

Invasiveness Into Matrigel

The degree of invasiveness into Matrigel or some other extracellular matrix constituent can be used as an assay to identify compounds that modulate metastatic colorectal cancer-associated sequences. Tumor cells exhibit a good correlation between malignancy and invasiveness of cells into Matrigel or some other extracellular matrix constituent. In this assay, tumorigenic cells are typically used as host cells. Expression of a tumor suppressor gene in these host cells would decrease invasiveness of the host cells.

Techniques described in Freshney (1994), supra, can be used. Briefly, the level of invasion of host cells can be measured by using filters coated with Matrigel or some other extracellular matrix constituent. Penetration into the gel, or through to the distal side of the filter, is rated as invasiveness, and rated histologically by number of cells and distance moved, or by prelabeling the cells with ¹²⁵I and counting the radioactivity on the distal side of the filter or bottom of the dish. See, e.g., Freshney (1984), supra.

Tumor Growth in vivo

Effects of metastatic colorectal cancer-associated sequences on cell growth can be tested in transgenic or immune-suppressed mice. Knock-out transgenic mice can be made, in which the metastatic colorectal cancer gene is disrupted or in which a metastatic colorectal cancer gene is inserted. Knock-out transgenic mice can be made by insertion of a marker gene or other heterologous gene into the endogenous metastatic colorectal cancer gene site in the mouse genome via homologous recombination. Such mice can also be made by substituting the endogenous metastatic colorectal cancer gene with a mutated version of the metastatic colorectal cancer gene, or by mutating the endogenous metastatic colorectal cancer gene, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

Alternatively, various immune-suppressed or immune-deficient host animals can be used. For example, genetically athymic “nude” mouse (see, e.g., Giovanella et al., J. Natl. Cancer Inst. 52:921 (1974)), a SCID mouse, a thymectomized mouse, or an irradiated mouse (see, e.g., Bradley et al., Br. J. Cancer 38:263 (1978); Selby et al., Br. J. Cancer 41:52 (1980)) can be used as a host. Transplantable tumor cells (typically about 10⁶ cells) injected into isogenic hosts will produce invasive tumors in a high proportions of cases, while normal cells of similar origin will not. In hosts which developed invasive tumors, cells expressing a metastatic colorectal cancer-associated sequences are injected subcutaneously. After a suitable length of time, preferably 4-8 weeks, tumor growth is measured (e.g., by volume or by its two largest dimensions) and compared to the control. Tumors that have statistically significant reduction (using, e.g., Student's T test) are said to have inhibited growth. Additionally, human tumor cells expressing the genes of the invention may be injected into immune compromised animals. Growth of these tumors, or xenografts, is compared to growth of similar human tumor cell that do not express the genes of the invention. These animals may also be used to binding assays and efficacy studies for therapeutic compounds that modulate metastatic colorectal cancer, such as antibodies or small molecules.

Polynucleotide Modulators of Metastatic Colorectal Cancer

Antisense Polynucleotides

In certain embodiments, the activity of a metastatic colorectal cancer-associated protein is downregulated, or entirely inhibited, by the use of antisense polynucleotide, i.e., a nucleic acid complementary to, and which can preferably hybridize specifically to, a coding mRNA nucleic acid sequence, e.g., a metastatic colorectal cancer protein mRNA, or a subsequence thereof. Binding of the antisense polynucleotide to the mRNA reduces the translation and/or stability of the mRNA.

In the context of this invention, antisense polynucleotides can comprise naturally-occurring nucleotides, or synthetic species formed from naturally-occurring subunits or their close homologs. Antisense polynucleotides may also have altered sugar moieties or inter-sugar linkages. Exemplary among these are the phosphorothioate and other sulfur containing species which are known for use in the art. Analogs are comprehended by this invention so long as they function effectively to hybridize with the metastatic colorectal cancer protein mRNA. See, e.g., Isis Pharmaceuticals, Carlsbad, Calif.; Sequitor, Inc., Natick, Mass.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known to those of skill in the art.

Antisense molecules as used herein include antisense or sense oligonucleotides. Sense oligonucleotides can, e.g., be employed to block transcription by binding to the anti-sense strand. The antisense and sense oligonucleotide comprise a single-stranded nucleic acid sequence (either RNA or DNA) capable of binding to target mRNA (sense) or DNA (antisense) sequences for metastatic colorectal cancer molecules. A preferred antisense molecule is for a metastatic colorectal cancer sequence in Tables 1-26, or for a ligand or activator thereof. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, e.g., Stein & Cohen (Cancer Res. 48:2659 (1988 and van der Krol et al. (BioTechniques 6:958 (1988)).

Ribozymes

In addition to antisense polynucleotides, ribozymes can be used to target and inhibit transcription of metastatic colorectal cancer-associated nucleotide sequences. A ribozyme is an RNA molecule that catalytically cleaves other RNA molecules. Different kinds of ribozymes have been described, including group I ribozymes, hammerhead ribozymes, hairpin ribozymes, RNase P, and axhead ribozymes (see, e.g., Castanotto et al., Adv. in Pharmacology 25: 289-317 (1994) for a general review of the properties of different ribozymes).

The general features of hairpin ribozymes are described, e.g., in Hampel et al., Nucl. Acids Res. 18:299-304 (1990); European Patent Publication No. 0 360 257; U.S. Pat. No. 5,254,678. Methods of preparing are well known to those of skill in the art (see, e.g., WO 94/26877; Ojwang et al., Proc. Natl. Acad. Sci. USA 90:6340-6344 (1993); Yamada et al., Human Gene Therapy 1:39-45 (1994); Leavitt et al., Proc. Natl. Acad. Sci. USA 92:699-703 (1995); Leavitt et al., Human Gene Therapy 5:1151-120 (1994); and Yamada et al., Virology 205: 121-126 (1994)).

Polynucleotide modulators of metastatic colorectal cancer may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a polynucleotide modulator of metastatic colorectal cancer may be introduced into a cell containing the target nucleic acid sequence, e.g., by formation of an polynucleotide-lipid complex, as described in WO 90/10448. It is understood that the use of antisense molecules or knock out and knock in models may also be used in screening assays as discussed above, in addition to methods of treatment.

Thus, in one embodiment, methods of modulating metastatic colorectal cancer in cells or organisms are provided. In one embodiment, the methods comprise administering to a cell an anti-metastatic colorectal cancer antibody that reduces or eliminates the biological activity of an endogenous metastatic colorectal cancer protein. Alternatively, the methods comprise administering to a cell or organism a recombinant nucleic acid encoding a metastatic colorectal cancer protein. This may be accomplished in any number of ways. In a preferred embodiment, e.g., when the metastatic colorectal cancer sequence is down-regulated in metastatic colorectal cancer, such state may be reversed by increasing the amount of metastatic colorectal cancer gene product in the cell. This can be accomplished, e.g., by overexpressing the endogenous metastatic colorectal cancer gene or administering a gene encoding the metastatic colorectal cancer sequence, using known gene-therapy techniques. In a preferred embodiment, the gene therapy techniques include the incorporation of the exogenous gene using enhanced homologous recombination (EHR), e.g., as described in PCT/US93/03868, hereby incorporated by reference in its entirety. Alternatively, e.g., when the metastatic colorectal cancer sequence is up-regulated in metastatic colorectal cancer, the activity of the endogenous metastatic colorectal cancer gene is decreased, e.g., by the administration of a metastatic colorectal cancer antisense nucleic acid.

In one embodiment, the metastatic colorectal cancer proteins of the present invention may be used to generate polyclonal and monoclonal antibodies to metastatic colorectal cancer proteins. Similarly, the metastatic colorectal cancer proteins can be coupled, using standard technology, to affinity chromatography columns. These columns may then be used to purify metastatic colorectal cancer antibodies useful for production, diagnostic, or therapeutic purposes. In a preferred embodiment, the antibodies are generated to epitopes unique to a metastatic colorectal cancer protein; that is, the antibodies show little or no cross-reactivity to other proteins. The metastatic colorectal cancer antibodies may be coupled to standard affinity chromatography columns and used to purify metastatic colorectal cancer proteins. The antibodies may also be used as blocking polypeptides, as outlined above, since they will specifically bind to the metastatic colorectal cancer protein.

Methods of Identifying Variant Metastatic Colorectal Cancer-Associated Sequences

Without being bound by theory, expression of various metastatic colorectal cancer sequences is correlated with metastatic colorectal cancer. Accordingly, disorders based on mutant or variant metastatic colorectal cancer genes may be determined. In one embodiment, the invention provides methods for identifying cells containing variant metastatic colorectal cancer genes, e.g., determining all or part of the sequence of at least one endogenous metastatic colorectal cancer genes in a cell. This may be accomplished using any number of sequencing techniques. In a preferred embodiment, the invention provides methods of identifying the metastatic colorectal cancer genotype of an individual, e.g., determining all or part of the sequence of at least one metastatic colorectal cancer gene of the individual. This is generally done in at least one tissue of the individual, and may include the evaluation of a number of tissues or different samples of the same tissue. The method may include comparing the sequence of the sequenced metastatic colorectal cancer gene to a known metastatic colorectal cancer gene, i.e., a wild-type gene.

The sequence of all or part of the metastatic colorectal cancer gene can then be compared to the sequence of a known metastatic colorectal cancer gene to determine if any differences exist. This can be done using any number of known homology programs, such as Bestfit, etc. In a preferred embodiment, the presence of a difference in the sequence between the metastatic colorectal cancer gene of the patient and the known metastatic colorectal cancer gene correlates with a disease state or a propensity for a disease state, as outlined herein.

In a preferred embodiment, the metastatic colorectal cancer genes are used as probes to determine the number of copies of the metastatic colorectal cancer gene in the genome.

In another preferred embodiment, the metastatic colorectal cancer genes are used as probes to determine the chromosomal localization of the metastatic colorectal cancer genes. Information such as chromosomal localization finds use in providing a diagnosis or prognosis in particular when chromosomal abnormalities such as translocations, and the like are identified in the metastatic colorectal cancer gene locus.

Administration of Pharmaceutical and Vaccine Compositions

In one embodiment, a therapeutically effective dose of a metastatic colorectal cancer protein or modulator thereof, is administered to a patient. By “therapeutically effective dose” herein is meant a dose that produces effects for which it is administered. The exact dose will depend on the purpose of the treatment, and will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999)). As is known in the art, adjustments for metastatic colorectal cancer degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

The administration of the metastatic colorectal cancer proteins and modulators thereof of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, e.g., in the treatment of wounds and inflammation, the metastatic colorectal cancer proteins and modulators may be directly applied as a solution or spray.

The pharmaceutical compositions of the present invention comprise a metastatic colorectal cancer protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that metastatic colorectal cancer protein modulators (e.g., antibodies, antisense constructs, ribozymes, small organic molecules, etc.) when administered orally, should be protected from digestion. It is also recognized that, after delivery to other sites in the body (e.g., circulatory system, lymphatic system, or the tumor site) the metastatic colorectal cancer modulators of the invention may need to be protected from excretion, hydrolisis, proteolytic digestion or modification, or detoxification by the liver. In all these cases, protection is typically accomplished either by complexing the molecule(s) with a composition to render it resistant to acidic and enzymatic hydrolysis, or by packaging the molecule(s) in an appropriately resistant carrier, such as a liposome or a protection barrier or by modifying the molecular size, weight, and/or charge of the modulator. Means of protecting agents from digestion degradation, and excretion are well known in the art.

The compositions for administration will commonly comprise a metastatic colorectal cancer protein modulator dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacologial Basis of Therapeutics (Hardman et al., eds., 1996)).

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art, e.g., Remington 's Pharmaceutical Science and Goodman and Gilhman, The Pharmacologial Basis of Therapeutics, supra.

The compositions containing modulators of metastatic colorectal cancer proteins can be administered for therapeutic or prophylactic treatments. In therapeutic applications, compositions are administered to a patient suffering from a disease (e.g., a cancer) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the agents of this invention to effectively treat the patient. An amount of modulator that is capable of preventing or slowing the development of cancer in a mammal is referred to as a “prophylactically effective dose.” The particular dose required for a prophylactic treatment will depend upon the medical condition and history of the mammal, the particular cancer being prevented, as well as other factors such as age, weight, gender, administration route, efficiency, etc. Such prophylactic treatments may be used, e.g., in a mammal who has previously had cancer to prevent a recurrence of the cancer, or in a mammal who is suspected of having a significant likelihood of developing cancer.

It will be appreciated that the present metastatic colorectal cancer protein-modulating compounds can be administered alone or in combination with additional metastatic colorectal cancer modulating compounds or with other therapeutic agent, e.g., other anti-cancer agents or treatments.

In numerous embodiments, one or more nucleic acids, e.g., polynucleotides comprising nucleic acid sequences set forth in Tables 1-26, such as antisense polynucleotides or ribozymes, will be introduced into cells, in vitro or in vivo. The present invention provides methods, reagents, vectors, and cells useful for expression of metastatic colorectal cancer-associated polypeptides and nucleic acids using in vitro (cell-free), ex vivo or in vivo (cell or organism-based) recombinant expression systems.

The particular procedure used to introduce the nucleic acids into a host cell for expression of a protein or nucleic acid is application specific. Many procedures for introducing foreign nucleotide sequences into host cells may be used. These include the use of calcium phosphate transfection, spheroplasts, electroporation, liposomes, microinjection, plasma vectors, viral vectors and any of the other well known methods for introducing cloned genomic DNA, cDNA, synthetic DNA or other foreign genetic material into a host cell (see, e.g., Berger & Kimmel, Guide to Molecular Cloning Techniques, Methods in Enzymology volume 152 (Berger), Ausubel et al., eds., Current Protocols (supplemented through 1999), and Sambrook et al., Molecular Cloning—A Laboratory Manual (2nd ed., Vol. 1-3, 1989.

In a preferred embodiment, metastatic colorectal cancer proteins and modulators are administered as therapeutic agents, and can be formulated as outlined above. Similarly, metastatic colorectal cancer genes (including both the full-length sequence, partial sequences, or regulatory sequences of the metastatic colorectal cancer coding regions) can be administered in a gene therapy application. These metastatic colorectal cancer genes can include antisense applications, either as gene therapy (i.e., for incorporation into the genome) or as antisense compositions, as will be appreciated by those in the art.

Metastatic colorectal cancer polypeptides and polynucleotides can also be administered as vaccine compositions to stimulate HTL, CTL and antibody responses. Such vaccine compositions can include, e.g., lipidated peptides (see, e.g., Vitiello, et al., J. Clin. Invest. 95:341 (1995)), peptide compositions encapsulated in poly(DL-lactide-co-glycolide) (“PLG”) microspheres (see, e.g., Eldridge, et al., Molec. Immunol. 28:287-294, (1991); Alonso et al., Vaccine 12:299-306 (1994); Jones et al., Vaccine 13:675-681 (1995)), peptide compositions contained in immune stimulating complexes (ISCOMS) (see, e.g., Takahashi et al., Nature 344:873-875 (1990); Hu et al., Clin Exp Immunol. 113:235-243 (1998)), multiple antigen peptide systems (MAPs) (see, e.g., Tam, Proc. Natl. Acad. Sci. U.S.A. 85:5409-5413 (1988); Tam, J. Immunol. Methods 196:17-32 (1996)), peptides formulated as multivalent peptides; peptides for use in ballistic delivery systems, typically crystallized peptides, viral delivery vectors (Perkus, et al., In: Concepts in vaccine development (Kaufmann, ed., p. 379, 1996); Chakrabarti, et al., Nature 320:535 (1986); Hu et al., Nature 320:537 (1986); Kieny, et al., AIDS Bio/Technology 4:790 (1986); Top et al., J. Infect. Dis. 124:148 (1971); Chanda et al., Virology 175:535 (1990)), particles of viral or synthetic origin (see, e.g., Kofler et al., J. Immunol. Methods. 192:25 (1996); Eldridge et al., Sem. Hematol. 30:16 (1993); Falo et al., Nature Med. 7:649 (1995)), adjuvants (Warren et al., Annu. Rev. Immunol. 4:369 (1986); Gupta et al., Vaccine 11:293 (1993)), liposomes (Reddy et al., J. Immunol. 148:1585 (1992); Rock, Immunol. Today 17:131 (1996)), or, naked or particle absorbed cDNA (Ulmer, et al., Science 259:1745 (1993); Robinson et al., Vaccine 11:957 (1993); Shiver et al., In: Concepts in vaccine development (Kaufmann, ed., p. 423, 1996); Cease & Berzofsky, Annu. Rev. Immunol. 12:923 (1994) and Eldridge et al., Sem. Hematol. 30:16 (1993)). Toxin-targeted delivery technologies, also known as receptor mediated targeting, such as those of Avant Immunotherapeutics, Inc. (Needham, Mass.) may also be used.

Vaccine compositions often include adjuvants. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, e.g., Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Vaccines can be administered as nucleic acid compositions wherein DNA or RNA encoding one or more of the polypeptides, or a fragment thereof, is administered to a patient. This approach is described, for instance, in Wolff et. al., Science 247:1465 (1990) as well as U.S. Pat. Nos. 5,580,859; 5,589,466; 5,804,566; 5,739,118; 5,736,524; 5,679,647; WO 98/04720; and in more detail below. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

For therapeutic or prophylactic immunization purposes, the peptides of the invention can be expressed by viral or bacterial vectors. Examples of expression vectors include attenuated viral hosts, such as vaccinia or fowlpox. This approach involves the use of vaccinia virus, e.g., as a vector to express nucleotide sequences that encode metastatic colorectal cancer polypeptides or polypeptide fragments. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover et al., Nature 351:456-460 (1991). A wide variety of other vectors useful for therapeutic administration or immunization e.g., adeno and adeno-associated virus vectors, retroviral vectors, Salmonella typhi vectors, detoxified anthrax toxin vectors, and the like, will be apparent to those skilled in the art from the description herein (see, e.g., Shata et al., Mol Med Today 6:66-71 (2000); Shedlock et al., J. Leukoc Biol 68:793-806 (2000); Hipp et al., In Vivo 14:571-85 (2000)).

Methods for the use of genes as DNA vaccines are well known, and include placing a metastatic colorectal cancer gene or portion of a metastatic colorectal cancer gene under the control of a regulatable promoter or a tissue-specific promoter for expression in a metastatic colorectal cancer patient. The metastatic colorectal cancer gene used for DNA vaccines can encode full-length metastatic colorectal cancer proteins, but more preferably encodes portions of the metastatic colorectal cancer proteins including peptides derived from the metastatic colorectal cancer protein. In one embodiment, a patient is immunized with a DNA vaccine comprising a plurality of nucleotide sequences derived from a metastatic colorectal cancer gene. For example, metastatic colorectal cancer-associated genes or sequence encoding subfragments of a metastatic colorectal cancer protein are introduced into expression vectors and tested for their immunogenicity in the context of Class I MHC and an ability to generate cytotoxic T cell responses. This procedure provides for production of cytotoxic T cell responses against cells which present antigen, including intracellular epitopes.

In a preferred embodiment, the DNA vaccines include a gene encoding an adjuvant molecule with the DNA vaccine. Such adjuvant molecules include cytokines that increase the immunogenic response to the metastatic colorectal cancer polypeptide encoded by the DNA vaccine. Additional or alternative adjuvants are available.

In another preferred embodiment metastatic colorectal cancer genes find use in generating animal models of metastatic colorectal cancer. When the metastatic colorectal cancer gene identified is repressed or diminished in metastatic tissue, gene therapy technology, e.g., wherein antisense RNA directed to the metastatic colorectal cancer gene will also diminish or repress expression of the gene. Animal models of metastatic colorectal cancer find use in screening for modulators of a metastatic colorectal cancer-associated sequence or modulators of metastatic colorectal cancer. Similarly, transgenic animal technology including gene knockout technology, e.g., as a result of homologous recombination with an appropriate gene targeting vector, will result in the absence or increased expression of the metastatic colorectal cancer protein. When desired, tissue-specific expression or knockout of the metastatic colorectal cancer protein may be necessary.

It is also possible that the metastatic colorectal cancer protein is overexpressed in metastatic colorectal cancer. As such, transgenic animals can be generated that overexpress the metastatic colorectal cancer protein. Depending on the desired expression level, promoters of various strengths can be employed to express the transgene. Also, the number of copies of the integrated transgene can be determined and compared for a determination of the expression level of the transgene. Animals generated by such methods find use as animal models of metastatic colorectal cancer and are additionally useful in screening for modulators to treat metastatic colorectal cancer.

Kits for Use in Diagnostic and/or Prognostic Applications

For use in diagnostic, research, and therapeutic applications suggested above, kits are also provided by the invention. In the diagnostic and research applications such kits may include any or all of the following: assay reagents, buffers, metastatic colorectal cancer-specific nucleic acids or antibodies, hybridization probes and/or primers, antisense polynucleotides, ribozymes, dominant negative metastatic colorectal cancer polypeptides or polynucleotides, small molecules inhibitors of metastatic colorectal cancer-associated sequences etc. A therapeutic product may include sterile saline or another pharmaceutically acceptable emulsion and suspension base.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The present invention also provides for kits for screening for modulators of metastatic colorectal cancer-associated sequences. Such kits can be prepared from readily available materials and reagents. For example, such kits can comprise one or more of the following materials: a metastatic colorectal cancer-associated polypeptide or polynucleotide, reaction tubes, and instructions for testing metastatic colorectal cancer-associated activity. Optionally, the kit contains biologically active metastatic colorectal cancer protein. A wide variety of kits and components can be prepared according to the present invention, depending upon the intended user of the kit and the particular needs of the user. Diagnosis would typically involve evaluation of a plurality of genes or products. The genes will be selected based on correlations with important parameters in disease which may be identified in historical or outcome data.

TABLE 1

| | |
|---|---|
| Pkey: | Unique Eas probeset identifier nnmber |
| ExAccn: | Exemplar Aceession number, Genbank accession nnmber |
| UnigeneID: | Unigene number |
| Unigene Title: | Unigene gene title |

| | | | | Ratio | |
|---|---|---|---|---|---|
| Pkey | ExAccn | UnigeneID | Unigene Title | Mets/BS | Top 3 expressing cell lines |
| 103989 | AA314779 | Hs.105484 | ESTs; Weakly similar to LITHOSTATHINE 1 | 15.77 | EB_cells, HT29_cells, HMEC |
| 101169 | L15533 | Hs.423 | pancreatitis-associated protein | 11.98 | HMEC (total RNA), Fibroblasts 2, |
| | | | | | Fibroblasts 2 |
| 101880 | M97925 | Hs.72887 | defensin; alpha 5; Paneth cell-specific | 9.24 | Fibroblasts 2, MB231_sells, MB-MDA-453 |
| 129462 | D84239 | Hs.111732 | IgG Fc binding protein | 8.57 | EB_cells, OVCAR_cells, HS578T_cells |
| 131676 | C20785 | Hs.30514 | ESTs | 7.43 | HMEC (total RNA), HMEC, Fibroblasts 2 |
| 131861 | D11925 | Hs.184245 | KIAA0929 protein Msx2 interacting nuclea | 7.15 | HMEC, HMEC (total RNA), Fibroblasts 2 |
| 118823 | N79237 | Hs.50813 | ESTs; Weakly similar to long chain fatty | 6.72 | HMEC, HMEC (total RNA), Lu_AD_H23 |
| 101107 | L08010 | Hs.4158 | regenerating islet-derived 1 beta (pancr | 6.33 | BT474_cells, Fibroblasts 2, MB231_cells |
| 103466 | Y00339 | Hs.155097 | carbonic anhydrase II | 6.18 | OVCAR_cells, MCF7, 293T_cells |
| 102306 | U33317 | Hs.711 | defensin; alpha 6; Paneth cell-specific | 5.67 | Fibroblasts 2, HMEC, HT29_cells |
| 126419 | AA451775 | Hs.129064 | H sapiens chromosome 19; cosmid F22162 | 5.14 | HS578T_cells, HMEC (total RNA), HMEC |
| 101198 | L21998 | Hs.315 | mucin 2; intestinal/tracheal | 5.1 | EB_cells, HT29_cells, MB231_cells |
| 107652 | AA010195 | Hs.52642 | ESTs; Weakly similar to !!!! ALU CLASS F | 4.94 | HMEC (total RNA), HMEC, EB_cells |
| 128145 | AI498467 | Hs.166669 | ESTs; Weakly similar to sodium bicarbona | 4.77 | HS578T_cells, HMEC, Lu_SQ_H520 |
| 110660 | H82117 | Hs.28043 | ESTs | 4.54 | HMEC, HS578T_cells, BT474_cells |
| 111669 | R19305 | Hs.110347 | H sapiens mRNA for alpha integrin bindin | 4.52 | HMEC, HS578T_cells, Caco2 |
| 124867 | R68971 | Hs.188500 | ESTs | 4.5 | HMEC, HMEC (total RNA), HS578T_cells |
| 127352 | AA416577 | Hs.189105 | ESTs | 4.41 | HMEC, HMEC (total RNA), |
| | | | | | MB-MDA-435s |
| 130736 | T99385 | Hs.18646 | EST | 4.29 | HMEC, EB_cells, HMEC (total RNA) |
| 128592 | AA470056 | Hs.113994 | ESTs; Weakly similar to alternatively sp | 4.18 | HMEC (total RNA), HMEC, Fibroblasts 2 |
| 108092 | AA045961 | Hs.169355 | ESTs; Weakly similar to | 4.04 | HMEC (total RNA), HMEC, Fibroblasts 2 |
| | | | TRANSCRIPTION RE | | |
| 133373 | S72487 | Hs.73946 | endothellal cell growth factor 1 (platel | 4.03 | EB_cells, HMEC, HMEC (total RNA) |
| 100572 | HG2271 | | Profilaggrin | 4.03 | HMEC (total RNA), HMEC, Fibroblasts 2 |
| 115775 | AA424030 | Hs.46627 | ESTs | 4.02 | HMEC, HMEC (total RNA), EB_cells |
| 120811 | AA346854 | Hs.52788 | fragile X mental retardation; autosomal | 4.01 | HMEC (total RNA), HMEC, Fibroblasts 2 |
| 111919 | R39926 | Hs.21031 | ESTs | 3.98 | EB_cells, HMEC (total RNA), HMEC |
| 117009 | H85422 | Hs.108556 | ESTs | 3.97 | HMEC (total RNA), HMEC, Fibroblasts 2 |
| 101124 | L10343 | Hs.112341 | protease inhibitor 3; skin-derived (SKAL | 3.89 | PC3_cells, RPWE_2, Caco2 |
| 106151 | AA424958 | Hs.33735 | ESTs | 3.88 | EBsells, HMEC, HMEC (total RNA) |
| 134733 | U03644 | Hs.89421 | CBF1 interacting corepressor | 3.88 | EB_cells, HMEC, HMEC (total RNA) |
| 131739 | AA449749 | Hs.31386 | ESTs; Highly similar to secreted apoptos | 3.87 | HS578T_cells, MB-MDA-435s, |
| | | | | | HT29_cells |
| 116311 | AA490469 | Hs.48752 | ESTs | 3.84 | HS578T_cells, HMEC, LNCaP_cells |
| 134174 | U05259 | Hs.79630 | CD79A antigen (immunoglobulin-associated | 3.83 | DU145_cells, Lu_AD_H23, MB231_cells |
| 106753 | AA476944 | Hs.7331 | ESTs | 3.82 | LNCaP_sells, Lu_SC_H345, DU145_cells |
| 104842 | AA039854 | Hs.8065 | H sapiens mRNA full length insert cDNA c | 3.78 | HS578T_cells, A549_cells, CALU6_cells |
| 129161 | N27334 | Hs.181780 | ESTs | 3.75 | HMEC (total RNA), HMEC, BT474_cells |
| 105675 | AA284767 | Hs.252808 | ESTs; Highly similar to pulmonary surfac | 3.75 | 293T_cells, PRSC_con, HT29_cells |
| 100547 | HG2149 | | Mucin (Gb:M57417) | 3.75 | HMEC (total RNA), HMEC, Fibroblasts 2 |
| 116887 | H65841 | Hs.186550 | ESTs | 3.73 | HS578T_cells, 293T_cells, HMEC |
| 113222 | T59670 | Hs.10615 | ESTs | 3.7 | HMEC, HS578T_cells, Caco2 |
| 118768 | N74467 | Hs.94304 | EST | 3.68 | HMEC, HS578T_cells, OVCAR_cells |
| 114542 | AA055768 | Hs.122578 | ESTs | 3.66 | EB_cells, MCF7, LNCaP_cells |
| 101640 | M58459 | Hs.180911 | ribasomal protein S4; Y-linked | 3.62 | DU145_cells, RPWE_2, A549_cells |
| 107754 | AA017462 | Hs.187571 | ESTs | 3.6 | HMEC (total RNA), Fibroblasts 2, |
| | | | | | Fibroblasts 2 |
| 104668 | AA007312 | Hs.183852 | ESTs; Weakly similarto polymerase [H.sa | 3.58 | HMEC (total RNA), HMEC, Fibroblasts 2 |
| 135377 | C21382 | Hs.99768 | H sapiens mRNA; cDNA DKFZp564J0323 | 3.56 | HMEC, HMEC (total RNA), EB_cells |
| | | | (from | | |
| 127083 | Z44079 | Hs.91608 | otoferlin | 3.53 | HMEC (total RNA), HMEC, Fibroblasts 2 |
| 102329 | U35407 | Hs.158084 | peroxisome receptor 1 | 3.51 | HMEC, HMEC (total RNA), EB_cells |
| 117882 | N50101 | Hs.124724 | ESTs; Weakly similar to coded for by C. | 3.47 | HMEC (total RNA), HMEC, EB_cells |
| 126405 | U46278 | Hs.122489 | ESTs | 3.46 | LNCaP_cells, MCF7, DU145_cells |
| 131378 | AA463886 | Hs.203910 | small glutamine-rich tetratricopeptide r | 3.45 | EB_cells, HMEC, HMEC (total RNA) |
| 111418 | R01084 | Hs.19081 | ESTs | 3.43 | HS578_Tcells, EB_cells, Lu_AD_H23 |
| 135398 | AA194075 | Hs.99908 | nuclear receptor coactivator 4 | 3.4 | HS578_Tcells, EB_cells, HMEC |
| 108710 | AA121960 | | zm24g9.sl Stratagene pancreas (#93728) H | 3.4 | EB_cells, HMEC, HMEC (total RNA) |
| | | | mRNA seq | | |
| 105437 | AA252191 | Hs.25199 | ESTs; Highly similar to match to ESTsAA | 3.38 | EB_cells, LNCaP_cells, RPWE_2 |
| 103448 | X99133 | Hs.204238 | lipocalin 2 (oncogene 24p3) | 3.38 | PC3_cells, EB_cells, HT29_sells |
| 130436 | M84526 | Hs.155597 | D component of complement (adipsin) | 3.37 | PRSC_con, EB_cells, Lu_AD_H23 |
| 112309 | R55021 | | yj76d5.s1 Soares breast 2NbHBst H sapien | 3.36 | EB_cells, HMEC, HMEC (total RNA) |
| 103211 | X73079 | Hs.205126 | polymeric immunoglobulin receptor | 3.35 | MB231_cells, HT29_cells, Lu_SC_H69 |
| 109012 | AA156576 | Hs.191466 | ESTs | 3.21 | EB_cells, HMEC, HMEC (total RNA) |
| 129989 | AF005887 | Hs.247433 | activating transcription factor 6 | 3.19 | HMEC (total RNA), HMEC, Lu_AD_H23 |
| 113466 | T86945 | Hs.16304 | ESTs | 3.18 | HMEC, MB231_cells, Caco2 |
| 103029 | X54489 | Hs.789 | GRO1 oncogene (melanoma growth stimulati | 3.16 | Lu_LC_H460, PC3_cells, Fibroblasts 2 |
| 109374 | AA218727 | Hs.210785 | ESTs; Highly similar to lbd1 [H.sapiens] | 3.13 | Caco2, A549_cells, MB231_cells |
| 131403 | R55750 | Hs.26455 | ESTs | 3.13 | HS578T_cells, HMEC, MB231_cells |
| 113420 | T83964 | Hs.15400 | ESTs | 3.11 | HMEC (total RNA), HMEC, EB_cells |
| 112532 | R69824 | Hs.28313 | ESTs | 3.11 | HMEC, HMEC (total RNA), EB_cells |
| 117905 | N50782 | Hs.231713 | EST | 3.11 | HMEC, HS578T_cells, Caco2 |
| 125349 | T87826 | Hs.164480 | ESTs | 3.1 | HS578T_cells, EB_cells, MB-MDA-435s |
| 107072 | AA609113 | Hs.177533 | H sapiens mRNA; cDNA DKFZp586N0318 | 3.1 | Lu_SC_H69, MB-MDA-453, MB231_cells |
| | | | (from | | |
| 118389 | N64583 | Hs.182365 | ESTs | 3.05 | HMEC, HMEC, LNCaP_cells |
| 117653 | N38970 | Hs.194214 | ESTs | 3.04 | HMEC, HMEC (total RNA), Fibroblasts 2 |
| 101082 | L05072 | Hs.80645 | interferon regulatory factor 1 | 3.04 | EB_cells, PRSC_con, DU145_cells |
| 126105 | H75323 | Hs.167614 | ESTs | 3.03 | HS578T_cells, HMEC (total RNA), HMEC |
| 120006 | W90108 | Hs.10848 | KIAA0187 gene product | 3.03 | HMEC, HMEC (total RNA), EB_cells |
| 127191 | AA297581 | | EST113160 Gall bladder I H sapiens cDNA | 3.02 | HMEC, Lu_AD_H23, Lu_SQ_H520 |
| 106899 | AA490107 | Hs.21753 | JM5 protein | 3.02 | EB_cells, HMEC (total RNA), HMEC |
| 112784 | R96306 | Hs.191290 | ESTs | 3.02 | EB_cells, HMEC, Lu_AD_358 |
| 113613 | T93337 | Hs.17167 | ESTs; Highly similar to LRR FLI-l intera | 3.02 | HMEC (total RNA), EB_cells, HMEC |
| 107631 | AA007230 | Hs.95026 | ESTs | 3.02 | Lu_SC_H345, HS578T_cells, Lu_LC_H460 |
| 101923 | S75256 | | HNL = neutrophil lipocalin [human, ovarian | 3.01 | PC3_cells, EB_cells, HT29_cells |
| 100695 | HG315T | | Beta-1-Glycoprotein 11, Pregnancy-Specif | 3.01 | Fibroblasts 2, Lu_AD_H23, MB-MDA-435s |
| 102523 | U53445 | Hs.15432 | downregulated in ovarian cancer 1 | 2.98 | PRSC_con, Fibroblasts 2, HMEC |
| 121588 | AA416615 | Hs.98242 | ESTs | 2.94 | HMEC, HS578T_cells, BT474_cells |
| 103714 | AA047055 | Hs.192943 | ESTs | 2.94 | HS578T_cells, EB_cells, HMEC |
| 104916 | AA056588 | Hs.16542 | ESTs | 2.93 | HMEC (total RNA), Fibroblasts 2, HMEC |
| 109928 | H05961 | Hs.26331 | ESTs | 2.92 | HMEC, MB231_cells, HS578T_cells |
| 104586 | R78309 | Hs.20787 | ESTs | 2.92 | Caco2, Lu_AD_358, Lu_AD_358 |
| 101236 | L29433 | Hs.47913 | coagulation factor X | 2.91 | HMEC, HS578T_cells, Caco2 |
| 134749 | L10955 | Hs.89485 | carbonic anhydrase IV | 2.9 | BT474_cells, MCF7, HMEC (total RNA) |
| 124703 | R07294 | Hs.109108 | solute carrier family 22 (organic cation | 2.9 | HMEC, HMEC (total RNA), MB-MDA-435s |
| 114108 | Z38431 | Hs.27038 | ESTs; Moderately similar to X-linkod ret | 2.89 | HMEC, HMEC (total RNA), EB_cells |
| 107857 | AA024687 | Hs.61208 | ESTs | 2.88 | HS578T_cells, MB231_cells, HMEC |
| 111586 | R10759 | Hs.15177 | ESTs | 2.88 | HS578T_cells, Lu_LC_H460, PRSC_con |
| 127553 | AA282433 | | H sapiens p60 katanin mRNA; complete cds | 2.87 | EB_cells, MB-MDA-435s, RPWE_2 |
| 129881 | AA458952 | Hs.197728 | ESTs; Weakly similar to ZINC FINGER PROT | 2.86 | EB_cells, PC3_cells, HMEC |
| 116852 | H65459 | Hs.38323 | ESTs | 2.85 | HMEC, Caco2, HS578T_cells |
| 133468 | X03068 | Hs.73931 | major histocompatibility complex; class | 2.82 | MB-MDA-435s, BT474_cells, HT29_cells |
| 130998 | C00810 | Hs.21970 | guanine nucleotide binding protein (G pr | 2.82 | LNCaP_cells, Lu_SC_H345, E8_cells |
| 124075 | H05741 | Hs.101643 | ESTs | 2.82 | HMEC, HS578T_cells, HT29_cells |
| 128108 | AI247422 | Hs.129966 | ESTs | 2.82 | HS578T_cells, Lu_LC_H1460, Lu_SC_H69 |
| 128096 | R15413 | Hs.164919 | ESTs; Highly similarto PROTEIN KINASE C | 2.8 | MB231_cells, Lu_AD_H23, RPWE_2 |
| 126619 | Z28861 | | HSBA7E032 STRATAGENE Human skeletal musc | 2.77 | HMEC, Lu_AD_H23, HMEC (total RNA) |
| | | | cDNA clone A7E03, mRNA seq. | | |
| 114418 | AA011383 | Hs.177313 | ESTs | 2.77 | HS578T_cells, EB_cells, MCF7 |
| 120383 | AA228030 | Hs.120234 | ESTs | 2.77 | EB_cells, Fibroblasts 2, HMEC (total RNA) |
| 126535 | H73017 | Hs.250723 | ESTs; Weakly similar to atrophin-1 relat | 2.76 | Fibroblasts 2, PRSC_con, DU145_cells |
| 119347 | T64349 | | yc10d0B.s1 Stratagene lung (#937210) H s | 2.76 | EB_cells, Lu_AD_H23, Lu_SC_H69 |
| 126219 | N36368 | Hs.141438 | ESTs; Moderately similar to similar to C | 2.76 | Lu_AD_H23, HMEC (total RNA), MB-MDA-435s |
| 125426 | R43963 | Hs.169355 | ESTs; Weakly similar to TRANSCRIPTION RE | 2.75 | HMEC, HMEC (total RNA), Lu_SC_H69 |
| 103005 | X52008 | Hs.2700 | glycine receptor; alpha 2 | 2.74 | HS578T_cells, HMEC, MB-MDA-453 |
| 109170 | AA180352 | Hs.191472 | ESTs | 2.74 | Fibroblasts 2, HMEC (total RNA), MB-MDA-435s |
| 101125 | L10373 | Hs.82749 | transmembrane 4 superfamily member 2 | 2.73 | Lu_LC_H460, 293T_cells, EB_cells |
| 130656 | Z20481 | Hs.17411 | KIAA0699 protein | 2.73 | HMEC (total RNA), HMEC, Fibroblasts 2 |
| 122933 | AA476728 | Hs.107537 | ESTs | 2.72 | HMEC, EB_cells, HMEC (total RNA) |
| 126033 | AA055978 | Hs.3807 | ESTs; Weakly similar to PHOSPHOLEMMAN PR | 2.71 | Lu_SC_H345, Lu_SC_H69, 293T_cells |
| 111644 | R16539 | Hs.223649 | EST; Moderately similar to Cd-7 Metallo | 2.71 | EB_cells, HMEC, HMEC (total RNA) |
| 133719 | AA033790 | Hs.75736 | apolipoprotein D | 2.71 | Caco2, Fibroblasts 2, MB-MDA-435s |
| 127555 | AA582324 | Hs.192857 | ESTs | 2.7 | HMEC, HS578T_cells, HMEC (total RNA) |
| 113321 | T70580 | Hs.13759 | ESTs | 2.69 | HMEC (total RNA), Fibroblasts 2, PRSC_con |
| 109326 | AA210719 | Hs.86414 | ESTs | 2.68 | MB-MDA-435s, HS578T_cells, Lu_SC_H69 |
| 135003 | H42527 | Hs.92832 | ESTs | 2.68 | HS578T_cells, EB_cells, PRSC_con |
| 103650 | Z70220 | | H.sapiens mRNA for 5′UTR for unknown pro | 2.68 | HMEC, HS578T_cells, PRSC_con |
| 111507 | R07728 | Hs.191218 | ESTs | 2.67 | HMEC (total RNA), HMEC, EB_cells |
| 117084 | H93081 | Hs.41829 | ESTs | 2.67 | HS578T_cells, HMEC, MB231_cells |
| 103975 | AA306264 | Hs.176403 | ESTs; Moderately similar to !!!! ALU SUB | 2.67 | DU145_cells, HS578T_cells, MB-MDA-435s |
| 132850 | R89741 | Hs.58215 | ESTs; Moderately similar to rhotekin [M. | 2.66 | HS578T_cells, EB_cells, 293T_cells |
| 121599 | AA416770 | Hs.98255 | EST | 2.61 | HMEC (total RNA), HMEC, EB_cells |
| 124230 | H63111 | Hs.6655 | ESTs | 2.6 | HMEC (total RNA), HMEC, Fibroblasts 2 |
| 114174 | Z39055 | Hs.27264 | ESTs; Moderately similar to !!!! ALU SUB | 2.58 | Caco2, MB-MDA-453, A549_cells |
| 128469 | T23724 | Hs.258677 | EST | 2.57 | Lu_LC_H460, Lu_SC_H69, MB-MDA-435s |
| 117399 | N26480 | Hs.43805 | lipoma HMGIC fusion partner-like 3 | 2.57 | HMEC, HMEC (total RNA), EB_cells |
| 129279 | AA460551 | Hs.184860 | ESTs; Weakly similar to EG:87B1.6 [D.mel | 2.57 | HS578T_cells, EB_cells, HT29_cells |
| 119817 | W74257 | Hs.159690 | ESTs | 2.57 | HMEC, HMEC (total RNA), Lu_SC_H69 |
| 114445 | AA019594 | Hs.250493 | ESTs; Weakly similar to KIAA0390 [H.sapi | 2.56 | HMEC, HT29_cells, Lu_LC_H460 |
| 120651 | AA287286 | Hs.99657 | ESTs | 2.55 | HMEC, HMEC (total RNA), Fibroblasts 2 |
| 105707 | AA291012 | Hs.37617 | ESTs; Weakly similar to KIAA0727 protein | 2.55 | HMEC (total RNA), EB_cells, BT474_cells |
| 128483 | T58588 | Hs.5148 | FLN29 gene product | 2.54 | HMEC, HS578T_cells, MB231_cells |
| 125890 | AA448739 | Hs.116708 | ESTs; Weakly similar to HYPOTHETICAL PRO | 2.54 | HMEC (total RNA), HMEC, OVCAR_cells |
| 134764 | M74715 | Hs.89560 | iduronidase; alpha-L- | 2.54 | BT474_cells, PRSC_con, HT29_cells |
| 113404 | T82323 | Hs.70337 | immunoglobulin superfamily; member 4 | 2.54 | Caco2, HS578T_cells, HMEC |
| 129128 | AA423854 | Hs.108812 | ESTs | 2.54 | BT474_cells, MB-MDA-435s, HMEC |
| 101428 | M19684 | Hs.184929 | protease inhibitor 1 (alpha-1-antitrypsi | 2.54 | HMEC, HT29_cells, HMEC (total RNA) |
| 103206 | X72755 | Hs.77367 | monokine induced by gamma interferon | 2.53 | Fibroblasts 2, MB231_cells, HMEC (total RNA) |
| 132273 | AA489716 | Hs.43658 | DKFZP586L151 protein | 2.53 | EB_cells, HMEC, HMEC (total RNA) |
| 108392 | AA075124 | | zm86a1.s1 Stratagene ovarian cancer (#93 | 2.52 | HMEC (total RNA), HMEC, HS578T_cells |
| | | | IMAGE:544776 3′, mRNA seq | | |
| 119508 | W37895 | Hs.45519 | ESTs | 2.52 | Lu_SC_H69, CALU6_cells, 293T_cells |
| 109828 | F13763 | Hs.19827 | ESTs | 2.52 | PRSC_log, PRSC_con, HS578T_cells |
| 135096 | N89775 | Hs.132390 | zinc finger protein 36 (KOX 18) | 2.51 | HMEC, HS578T_cells, HT29_cells |
| 130860 | U66061 | Hs.241395 | protease; seine; 1 (trypsin 1) | 2.51 | OVCAR_cells, MB231_cells, PC3_cells |
| 105725 | AA292228 | Hs.199791 | STAT induced STAT inhibitor 3 | 2.51 | HS578T_cells, HT29_cells, HMEC |
| 110427 | H48579 | Hs.36275 | EST | 2.51 | HS578T_sells, Caco2, Lu_LC_H460 |
| 123762 | AA610013 | Hs.244553 | EST | 2.51 | HMEC (total RNA), HMEC, Fibroblasts 2 |
| 126406 | AA034096 | | zi06f05.r1 Soares_fetal_liver_spleen_1NF | 2.5 | Lu_AD_H23, HS578T_cells, Lu_AD_358 |
| | | | IMAGE:430017 5′, mRNA seq. | | |
| 129751 | AA346065 | Hs.111286 | KIAA0714 protein | 2.5 | HMEC, HS578T_cells, Fibroblasts 2 |
| 121704 | AA418743 | Hs.98306 | ESTs | 2.5 | EB_cells, HMEC (total RNA), HMEC |
| 112595 | R77783 | Hs.22404 | protease; serine; 12 (neurotrypsin; moto | 2.5 | Fibroblasts 2, EB_cells, PRSC_son |
| 108499 | AA083103 | | zn1b12.s1 Stratagene hNT neuron (#937233 | 2.5 | LNCaP_cells, MB-MDA-453, HMEC |
| | | | IMAGE:5477 3′, mRNA seq | | |
| 131968 | AA151333 | Hs.36029 | ESTs; Highly similar to basic helix-loop | 2.5 | Fibroblasts 2, A549_cells, 293T_cells |
| 112665 | R85661 | Hs.221447 | ESTs | 2.48 | Lu_AD_H23, HMEC, Lu_LC_H460 |
| 115764 | AA421562 | Hs.91011 | anterior gradient 2 (Xenepus laevis) hom | 2.48 | EB_cells, Caco2, MCF7 |
| 105959 | AA405540 | Hs.7001 | ESTs | 2.48 | OVCAR_cells, BT474_cells, Caco2 |
| 125804 | R79519 | Hs.16899 | ESTs | 2.48 | HMEC (total RNA), EB_cells, HMEC |
| 110102 | H16681 | Hs.180950 | guanine nucleotide binding protein (G pr | 2.46 | HS578T_cells, HMEC, OVCAR_cells |
| 104680 | AA009809 | Hs.37599 | ESTs | 2.46 | HMEC, HS578T_cells, Caco2 |
| 132339 | D80030 | Hs.45127 | chondroitin sulfate proteoglycan 5 (neur | 2.45 | OVCAR_cells, 293T_cells, HMEC (total RNA) |
| 121712 | AA419116 | Hs.193663 | ESTs; Weakly similar to !!!! ALU SUBFAMI | 2.45 | Lu_SQ_H520, Lu_AD_H23 Lu_SC_H69 |
| 129226 | M96843 | Hs.180919 | inhibitor of DNA binding 2; dominant neg | 2.44 | MB-MDA-453, 293T_cells, Caco2 |
| 128731 | AF005271 | Hs.104555 | neuropeptide FF-amide peptide precursor | 2.43 | HMEC, HMEC (total RNA), EB_cells |
| 106670 | AA461174 | Hs.5943 | ESTs | 2.43 | EB_cells, HS578T_cells, Lu_SC_H69 |
| 119306 | T26914 | Hs.132785 | EAP30 subunit of ELL complex | 2.43 | EB_cells, HMEC (total RNA), HMEC |
| 133507 | X74295 | Hs.74369 | integrin; alpha 7 | 2.42 | Fibroblasts 2, Caco2, EB_cells |
| 125713 | AA367905 | Hs.77356 | transferrin receptor (p90; CD71) | 2.41 | HS578T_cells, Fibroblasts 2, Lu_AD_H23 |
| 107438 | W27841 | Hs.17118 | ESTs; Weakly similar to B0025.2 ]C.elega | 2.41 | HMEC, HS578T_cells, MB231_cells |
| 101784 | M83186 | Hs.114346 | cytochrome c oxidase subunit VIIa polype | 2.41 | Fibroblasts 2, PRSC_con, PRSC_log |
| 134578 | AA194724 | Hs.182418 | endonuclease G | 2.4 | EB_cells, HMEC, Lu_AD_H23 |
| 125105 | T95642 | Hs.189759 | ESTs | 2.4 | EB_cells, A549_cells, HS578T_cells |
| 127067 | AA380418 | Hs.88012 | SHP2 interacling transmembrane adaptor | 2.4 | HMEC, HMEC (total RNA), EB_cells |
| 113118 | T47906 | Hs.220512 | ESTs | 2.39 | MB-MDA-435s, HS578T_cells, HMEC |
| 104791 | AA029046 | Hs.30377 | ESTs; Moderately similar to dAMP inducib | 2.39 | LNCaP_cells, OVCAR_cells, PC3_cells |
| 115833 | AA428269 | Hs.125035 | ESTs | 2.38 | Caco2, LNCaP_cells, CALU6_cells |
| 132223 | R77451 | Hs.4245 | ESTs; Weakly similar to similar to S. ce | 2.38 | HMEC, HMEC (total RNA), EB_cells |
| 115836 | AA428863 | Hs.89388 | ESTs | 2.38 | HS578T_cells, HMEC, PRSC_con |
| 101891 | S45630 | Hs.1940 | crystallin; alpha B | 2.38 | HS578T_cells, OVCAR_cells, Lu_LC_H460 |
| 132894 | D82422 | Hs.5944 | ESTs | 2.37 | Caco2, MB-MDA-453, HT29_cells |
| 106939 | AA496048 | Hs.26570 | ESTs | 2.35 | LNCaP_cells, 293T_cells, EB_cells |
| 131104 | W27770 | Hs.258721 | ESTs | 2.35 | HMEC (total RNA), HMEC, HT29_cells |
| 122355 | AA443789 | Hs.189324 | ESTs | 2.34 | HMEC (total RNA), HMEC, EB_cells |
| 119343 | T62873 | | yc3d2.s1 Stratagene lung (#93721) H sapi | 2.34 | HS578T_cells, Lu_SC_H69, HT29_cells |
| | | | to contains Alu repetitive element;, mR | | |
| 115442 | AA284722 | Hs.89121 | H sapiens mRNA; chromosome 1 specific tr | 2.33 | Lu_AD_H23, HMEC (total RNA), BT474_cells |
| 134286 | T69384 | Hs.68398 | period (Drosophila) homolog 1 | 2.33 | HMEC, HMEC (total RNA), MB231_cells |
| 125465 | AI375276 | Hs.158732 | ESTs | 2.33 | HMEC (total RNA), EB_cells, HMEC |
| 127449 | AI421866 | Hs.75722 | ribophorin II | 2.33 | Lu_AD_H23, HMEC (total RNA), HMEC |
| 110225 | H23927 | Hs.222381 | ESTs | 2.33 | HS578T_cells, HMEC, Lu_LC_H460 |
| 119930 | W86471 | Hs.151624 | hypocretin (orexin) receptor 2 | 2.32 | HMEC, HMEC (total RNA), EB_cells |
| 125958 | AI073357 | Hs.12311 | H sapiens clone 23570 mRNA seq | 2.32 | MB231_cells, HMEC (total RNA), HMEC |
| 119746 | W70279 | Hs.221189 | ESTs; Weakly similar to 15-HYDROXYPROSTA | 2.32 | HMEC, HS578T_cells, MB231_cells |
| 108874 | AA134112 | Hs.107187 | H sapiens DNA seq from cosmid ICK072IQ o | 2.32 | Caco2, PRSC_con, LNCaP_cells |
| | | | L12 LIKE protein in an intron of the HS | | |
| 127368 | AA434362 | Hs.193326 | ESTs | 2.32 | HMEC (total RNA), HS578T_cells, HMEC |
| 120437 | AA243427 | Hs.104311 | ESTs | 2.32 | HMEC (total RNA), HMEC, MB-MDA-435s |
| 119867 | W80852 | Hs.250696 | KDEL (Lys-Asp-Glu-Leu) endoplasmic retic | 2.32 | Fibroblasts 2, HS578T_cells, MB-MDA-435s |
| 131205 | J02947 | Hs.2420 | superoxide dismutase 3; extracellular | 2.32 | PRSC_con, EB_cells, Lu_AD_358 |
| 133710 | X76057 | Hs.75694 | mannose phosphate isomerase | 2.31 | 293T_cells, LNCaP_cells, RPWE_2 |
| 104834 | AA039331 | Hs.16323 | ESTs; Weakly similar to GAGE-7 [H.sapien | 2.31 | Caco2, HS578T_cells, HMEC |
| 113186 | T56048 | Hs.189674 | ESTs | 2.31 | HMEC, Fibroblasts 2, HMEC (total RNA) |
| 113462 | T86826 | Hs.142528 | ESTs | 2.31 | PC3_cells, HS578T_cells, HMEC |
| 104743 | AA0211157 | Hs.33619 | ESTs | 2.3 | HMEC (total RNA), HMEC, OVCAR_cells |
| 129687 | Y00097 | Hs.118796 | annexin A6 | 2.3 | PRSC_log, PRSC_con, HS578T_cells |
| 111573 | R10305 | Hs.185683 | ESTs | 2.3 | HMEC, HMEC (total RNA), EB_cells |
| 117523 | N32626 | Hs.145532 | ESTs; Weakly similar to Gag polyprotein | 2.29 | EB_cells, Fibroblasts 2, HS578T_cells |
| 115540 | AA349954 | Hs.56281 | ESTs; Weakly similar to ASB-1 protein [H | 2.29 | Fibroblasts 2, BT474_cells, MB231_cells |
| 101622 | M55621 | Hs.151513 | mannosyl (alpha-1;3-)-glycoprotein beta- | 2.29 | PRSC_con, RPWE_2, PRSC_log |
| 103535 | Y13620 | Hs.122607 | B-cell CLL/lymphoma 9 | 2.28 | Lu_SC_H69, Lu_AD_358, Lu_AD_H23 |
| 127482 | AI337294 | Hs.155014 | ESTs | 2.28 | HS578T_cells, 293T_cells, CALU6_cells |
| 104297 | D31111 | Hs.106005 | ESTs; Highly similar to NY-REN-50 antige | 2.27 | EB_cells, DU145_cells, HT29_cells |
| 112318 | R55470 | Hs.11067 | ESTs | 2.27 | MB-MDA-453, LNCaP_cells, OVCAR_cells |
| 101877 | M97496 | Hs.778 | guanylate cyclase activator 1B (retina) | 2.27 | HT29_cells, BT474_cells, Caco2 |
| 100760 | HG3576 | | Major Histocompatibility Complex, Class | 2.26 | MB-MDA-435s, MB231_cells, BT474_cells |
| 102362 | U39412 | Hs.75932 | N-ethylmaleimide-sensitive factor attach | 2.26 | LNCaP_cells, MB-MDA-453, Caco2 |
| 106142 | AA424590 | Hs.239631 | Golgi transport complex protein (90 kDa) | 2.26 | HMEC, HS578T_cells, Caco2 |
| 101461 | M22430 | Hs.76422 | phospholipase A2; group IIA (platelets; | 2.26 | LNCaP_cells, BT474_cells, Caco2 |
| 119336 | T55340 | Hs.208238 | ESTs | 2.26 | HS578T_cells, EB_cells, HMEC |
| 127619 | AA627122 | Hs.163787 | ESTS | 2.25 | Lu_SQ_H520, Lu_LC_H460, Lu_SC_H69 |
| 104113 | AA427510 | Hs.181202 | ESTs; Weakly similar to Wiscott-Aldrich | 2.25 | MB-MDA-435s, Fibroblasts 2, HMEC (total RNA) |
| 131219 | C00476 | Hs.24395 | small inducible cytokine subfamily B (Cy | 2.25 | Lu_SQ_H520, BT474_cells, Fibroblasts 2 |
| 118915 | N91481 | Hs.54713 | ESTs | 2.25 | HMEC (total RNA), HMEC, MCF7 |
| 127556 | AA679831 | Hs.190228 | ESTs | 2.24 | HS578T_cells, EB_cells, HMEC |
| 128700 | U59286 | Hs.103982 | small inducible cytokine subfamily B (Cy | 2.24 | HMEC, HS578T_cells, Fibroblasts 2 |
| 113674 | T96374 | Hs.5753 | Inositol(myo)-1(or 4)-monophosphatase 2 | 2.24 | A549_cells, DU145_cells, Lu_AD_358 |
| 133085 | M73720 | Hs.646 | carboxypeptidase A3 (mast cell) | 2.24 | HS578T_cells, Fibroblasts 2, HT29_cells |
| 106017 | AA411882 | Hs.26268 | ESTs | 2.24 | MB-MDA-453, OVCAR_cells, 293T_cells |
| 100582 | HG2348 | | Peptide Yy | 2.24 | HMEC, HS578T_cells, HMEC (total RNA) |
| 134811 | N66357 | Hs.89761 | ATP synthase; H+ transporting; mitochond | 2.23 | Lu_SQ_H520, LNCaP_cells, Lu_AD_H23 |
| 102543 | U57627 | Hs.234776 | oculocerebrorenal syndrome of Lowe | 2.23 | 293T_cells, EB_cells, LNCaP_cells |
| 127357 | AA452788 | | zx39g11.r1 Soares_total_fetus_Nb2HF8_9w | 2.23 | HS578T_cells, RPWE_2, HMEC (total RNA) |
| | | | IMAGE:788900 5′, mRNA seq. | | |
| 135288 | AA402930 | Hs.97876 | ESTs | 2.23 | HS578T_cells, 293T_cells, OVCAR_cells |
| 105581 | AA278850 | Hs.28891 | ESTs; Weakly similar to !!!! ALU SUBFAMI | 2.23 | BT474_cells, BT474_cells, MB231_cells |
| 103812 | AA137107 | Hs.124094 | ESTs; Weakly similarto NFAT1-A [M.muscu | 2.23 | Lu_SC_H345, Lu_AD_H23, PRSC_con |
| 117016 | H87171 | Hs.52170 | ESTs | 2.22 | Fibroblasts 2, Lu_LC_H460, HMEC (total RNA) |
| 114607 | AA079342 | Hs.129057 | breast carcinoma amplified seq 1 | 2.22 | BT474_cells, HT29_cells, HT29_cells |
| 134000 | U29091 | Hs.7833 | selenium binding protein 1 | 2.22 | LNCaP_cells, MB-MDA-453, BT474_cells |
| 111069 | N58461 | Hs.22036 | ESTs | 2.22 | HMEC, Lu_SC_H345, HS578T_cells |
| 129048 | L27670 | Hs.108287 | intercellular adhesion molecule 4; Lands | 2.21 | Lu_AD_H23, HS578T_cells, Lu_SQ_H520 |
| 124995 | T52700 | Hs.110044 | ESTs | 2.2 | Caco2, MB-MDA-453, HT29_cehls |
| 116678 | F05063 | Hs.251736 | ESTs | 2.2 | HS578T_cells, BT474_cells, 293T_cells |
| 118222 | N62263 | Hs.48501 | EST | 2.2 | HS578T_cells, BT474_cells, MB231_cells |
| 127888 | AI149662 | Hs.143590 | ESTs | 2.19 | BT474_cells, CALU6_cells, MB231_cells |
| 113790 | W33178 | Hs.26912 | ESTs | 2.19 | HMEC, HMEC (total RNA), Fibroblasts 2 |
| 100097 | AF002224 | | H sapiens Angelman Syndrome Gene, E6-AP | 2.19 | HS578T_cells, CALU6_cells, 293T_cells |
| | | | from promoter P1, 5′UTR | | |
| 109151 | AA176800 | Hs.73452 | ESTs | 2.19 | CALU6_cells, Lu_AD_H23, Lu_SC_H69 |
| 135368 | AA086057 | Hs.9964 | ribosomal protein; mitochondrial; S12 | 2.19 | OVCAR_cells, A549_cells, Lu_AD_H23 |
| 109016 | AA156936 | Hs.58069 | ESTs; Highly similar to type II cAMP-dep | 2.19 | HS578T_cells, BT474_cells, A549_cells |
| 124300 | H92575 | Hs.105959 | ESTs; Weakly similar to !!!! ALU SUBFAMI | 2.18 | Lu_AD_358, Lu_SC_H69, Lu_SC_H345 |
| 123450 | AA598913 | Hs.111207 | ESTs | 2.18 | HMEC (total RNA), HMEC, MB-MDA-435s |
| 117435 | N27628 | | yw50b08.s1 Weizmann Olfactory Epithelium | 2.18 | LNCaP_cells, DU145_cells, Lu_SQ_H520 |
| 119860 | W80709 | Hs.58485 | ESTs | 2.18 | HS578T_cells, MB231_cells, Caco2 |
| 123833 | AA620717 | Hs.112889 | ESTs | 2.18 | Lu_AD_H23, Lu_SQ_H520, Lu_AD_358 |
| 107936 | AA029446 | Hs.53115 | ESTs | 2.17 | Caco2, 293T_cells, 293T_cells |
| 119380 | T83659 | Hs.184407 | ESTs | 2.16 | Lu_AD_H23, Lu_AD_358, PRSC_con |
| 114066 | Z38152 | Hs.26920 | ESTs | 2.15 | HMEC (total RNA), HMEC, EB_cells |
| 128748 | T59001 | Hs.10475 | ESTs | 2.15 | HMEC, HT29_cells, MB231_cells |
| 130414 | M21121 | Hs.241392 | small inducible cytokine A5 (RANTES) | 2.15 | HS578T_cells, PC3_cells, A549_cells |
| 123490 | AA599723 | | TAP binding protein (tapasin) | 2.15 | HS578T_cells, EB_cells, Lu_SC_H69 |
| 112588 | R77302 | Hs.20226 | ESTs | 2.14 | HMEC (total RNA), HMEC, Fibroblasts 2 |
| 110548 | H58715 | Hs.14706 | ESTs | 2.14 | HMEC, HMEC (total RNA), HT29_cells |
| 101581 | M34996 | Hs.198253 | major histocompatibility complex; class | 2.14 | MB-MDA-435s, HMEC, HMEC |
| 115248 | AA278887 | Hs.194530 | ESTs; Weakly similarto unknown [H.sapie | 2.14 | HT29_cells, BT474_cells CALU6_cells |
| 105619 | AA280810 | Hs.24003 | ESTs; Moderately similar to LEYDIG CELL | 2.14 | Lu_SQ_H520, MB-MDA-435s, LNCaP_cells |
| 128058 | AI126617 | Hs.132449 | ESTs | 2.14 | HS578T_cells, EB_cells, HMEC (total RNA) |
| 134573 | AA442125 | Hs.171873 | ESTs; Weakly similar to PUTATIVE STEROID | 2.14 | EB_cells, MB231_cells, Caco2 |
| 134863 | AA353903 | Hs.183373 | ATX1 (antioxidant protein 1; yeast) homo | 2.14 | Lu_SC_H345, HT29_cells, BT474_cells |
| 128811 | H17317 | Hs.169100 | ESTs; Weakly similar to HPBRII-7 protein | 2.13 | Caco2, Lu_SC_H345, EB_cells |
| 112368 | R59371 | Hs.26653 | EST | 2.13 | HMEC, HMEC (total RNA), Lu_SQ_H520 |
| 108395 | AA075144 | | zm86f6.s1 Stratagene ovarian cancer (#93 | 2.13 | HMEC (total RNA), HMEC, OVCAR_cells |
| | | | gb:X1664 TRANSLATIONALLY CONTROLLED TUM | | |
| 129611 | D45680 | Hs.11614 | ESTs | 2.13 | HMEC, HS578T_cells, Caco2 |
| 101253 | L34355 | Hs.99931 | sarcoglycan; alpha (50 kD dystrophin-asso | 2.12 | HS578T_cells, OVCAR_cells, CALU6_cells |
| 126701 | AA515212 | Hs.202590 | ESTs; Weakly similar to mucin glycoprote | 2.12 | EB_cells, Lu_AD_H23, Lu_AD_H23 |
| 111628 | R15825 | Hs.4014 | KIAA0940 protein; Huntingtin interacting | 2.12 | A549_cells, BT474_cells, MB-MDA-435s |
| 108675 | AA115240 | Hs.61816 | ESTs | 2.12 | Lu_AD_H23, MB-MDA-453, PRSC_con |
| 127131 | Z44658 | Hs.105460 | DKFZP564O0823 protein | 2.12 | EB_cells, Lu_SC_H69, Lu_SC_H69 |
| 109590 | F02465 | Hs.27281 | ESTs | 2.12 | HMEC, HS578T_cells, HMEC (total RNA) |
| 116539 | D12124 | Hs.242890 | EST | 2.12 | Lu_AD_H23, Caco2, BT474_cells |
| 112117 | R45402 | Hs.23789 | ESTs | 2.12 | EB_cells, Lu_AD_H23, Lu_SQ_H520 |
| 126367 | AA477929 | Hs.25584 | ESTs | 2.12 | Lu_SC_H69, Lu_AD_H23, Lu_AD_358 |
| 135252 | U62966 | Hs.97207 | solute carrier family 28 (sodium-coupled | 2.11 | MB-MDA-435s, 293T_cells, CALU6_cells |
| 117565 | N34301 | Hs.248426 | EST | 2.11 | HMEC, HS578T_cells, MB231_cells |
| 129430 | AA258842 | Hs.197877 | H sapiens clone 23777 putative transmemb | 2.11 | HS578T_cells, Lu_AD_358, MB-MDA-435s |
| 120256 | AA169801 | | sema domain; immunoglobulin domain (lg); | 2.11 | HMEC, HMEC (total RNA), EB_cells |
| 134169 | D20342 | Hs.178137 | transducer of ERBB2; 1 (TOB1) | 2.11 | HMEC (total RNA), 293T_cells, OVCAR_cells |
| 130397 | AA487452 | Hs.155344 | DNA fragmentation factor; 45 kD; alpha s | 2.11 | 293T_cells, Caco2, Lu_AD_H23 |
| 132859 | D20925 | Hs.5842 | ESTs | 2.11 | HMEC (total RNA), Fibroblasts 2, HMEC |
| 117633 | N36404 | Hs.44807 | ESTs | 2.11 | HMEC, Caco2, HS578T_cells |
| 125003 | T59442 | Hs.100445 | ESTs | 2.11 | MB-MDA-435s, HMEC (total RNA), HT29_cells |
| 125329 | AA825437 | Hs.58875 | ESTs | 2.11 | HS578T_cells, PRSC_con, PRSC_log |
| 114065 | Z38149 | Hs.134015 | uronyl 2-sulfotransferase | 2.11 | MB-MDA-435s, 293T_cells, PRSC_con |
| 120718 | AA292747 | Hs.97296 | ESTs | 2.11 | HT29_cells, Lu_AD_H23, Lu_SC_H69 |
| 133869 | T49444 | Hs.77031 | Sp2 transcription factor | 2.1 | Lu_LC_H460, Lu_AD_358, RPWE_2 |
| 135351 | AA430179 | Hs.9933 | putative Ac-like transposon | 2.1 | HS578T_cells, EB_cells, HMEC |
| 110973 | N51529 | Hs.118047 | ESTs | 2.09 | EB_cells, HS578T_cells, MCF7 |
| 131879 | AA017161 | Hs.33792 | ESTs | 2.09 | HMEC (total RNA), MB231_cells, BT474_cells |
| 116656 | F03935 | Hs.241640 | EST | 2.09 | HS578T_cells, Lu_LC_H460, Lu_SC_H69 |
| 120311 | AA194074 | Hs.193401 | ESTs | 2.09 | OVCAR_cells, HMEC (total RNA), HMEC |
| 108024 | AA040433 | Hs.61898 | DKFZP586N2124 protein | 2.09 | HMEC (total RNA), BT474_cells, HT29_cells |
| 105871 | AA399633 | Hs.24872 | ESTs | 2.09 | Fibroblasts 2, A549_cells, HS578T_cells |
| 120206 | Z40805 | Hs.91668 | ESTs | 2.09 | BT474_cells, MB-MDA-453, EB_cells |
| 112333 | R56222 | Hs.26514 | ESTs | 2.09 | Lu_AD_H23, Fibroblasts 2, Lu_LC_H460 |
| 116746 | H04811 | Hs.79027 | ESTs | 2.08 | MB-MDA-435s, HMEC (total RNA), Lu_SC_H345 |
| 121529 | AA412257 | Hs.98121 | ESTs | 2.08 | HMEC, HMEC (total RNA), HS578T_cells |
| 105592 | AA279337 | Hs.180549 | ESTs; Highly similar to R26660_1; partia | 2.08 | LNCaP_cells, PRSC_log, PRSC_log |
| 108582 | AA088231 | Hs.91732 | ESTs | 2.08 | HS578T_cells, Lu_SC_H345, Lu_SC_H69 |
| 123197 | AA489250 | Hs.59403 | serine palmitoyltransferase; subunit II | 2.08 | EB_cells, Lu_SC_H69, Lu_SC_H345 |
| 134965 | J05480 | Hs.92 | protein phosphatase 3 (formerly 2B); cat | 2.08 | LNCaP_cells, MB-MDA-435s, HMEC |
| 123856 | AA620814 | Hs.144959 | ESTs | 2.08 | HS578T_cells, BT474_cells, BT474_cells |
| 132058 | AA251737 | Hs.172818 | Apg12 (autophagy 12; S. cerevisiae)-like | 2.07 | HS578T_cells, MCF7, HMEC |
| 126476 | R94666 | Hs.195155 | ESTs; Weakly similar to transporter prot | 2.07 | PRSC_log, Lu_LC_H460, RPWE_2 |
| 106087 | AA418740 | Hs.21111 | ESTs | 2.07 | OVCAR_cells, A549_cells, Lu_AD_H23 |
| 103802 | AA122003 | Hs.62954 | ferritin; heavy polypeptide 1 | 2.07 | HMEC, HMEC (total RNA), HS578T_cells |
| 125633 | AA908225 | Hs.126641 | ESTs | 2.07 | EB_cells, Flbroblasts 2, Lu_SC_H69 |
| 112817 | R98491 | Hs.14584 | ESTs | 2.07 | HMEC, HMEC (total RNA), Fibroblasts 2 |
| 111050 | N56984 | Hs.74335 | heat shock 90 kD protein 1; beta | 2.07 | LNCaP_cells, DU145_cells, 293T_cells |
| 133072 | AA425294 | Hs.64322 | ESTs; Weakly similar to Closely related | 2.07 | LNCaP_cells, MB-MDA-453, Caco2 |
| 118270 | N62868 | Hs.48653 | ESTs | 2.07 | HMEC (total RNA), HMEC, EB_cells |
| 105035 | AA128406 | Hs.8859 | ESTs | 2.07 | LNCaP_cells, PC3_cells, EB_cells |
| 102337 | U36922 | | Human fork head domain protein (FKHR) mR | 2.07 | 293T_cells, HMEC, HT29_cells |
| 109687 | F09380 | Hs.182859 | lifeguard | 2.06 | BT474_cells, BT474_cells, Lu_AD_H23 |
| 109802 | F10789 | Hs.12439 | ESTs | 2.06 | EB_cells, EB_cells, Caco2 |
| 128103 | AA905960 | Hs.48516 | ESTs | 2.06 | HT29_cells, HMEC (total RNA), HMEC |
| 128278 | AI018343 | Hs.131275 | ESTs | 2.06 | PRSC_con, Lu_SC_H345, HS578T_cells |
| 131873 | H39997 | Hs.33716 | ESTs | 2.08 | HMEC (total RNA), HMEC, EB_cells |
| 122683 | AA455528 | Hs.96772 | ESTs | 2.05 | LNCaP_cells, Lu_AD_H23, HS578T_cells |
| 128066 | AA884838 | Hs.189171 | ESTs | 2.05 | HMEC, HUEC (total RNA), Fibroblasts 2 |
| 131451 | N28028 | Hs.26968 | H sapiens mRNA from chromosome 5q21-22; | 2.05 | MB-MDA-435s, Lu_LC_H460, Lu_SQ_H520 |
| 120887 | AA365644 | Hs.97043 | ESTs | 2.05 | HS578T_cells, PRSC_con, HMEC |
| 103966 | AA303166 | Hs.127270 | ESTs | 2.05 | HMEC (total RNA), LNCaP_cells, PC3_cells |
| 105861 | AA399260 | Hs.28454 | ESTs | 2.05 | Fibroblasts 2, HMEC (total RNA), EB_cells |
| 104627 | AA001976 | Hs.19603 | ESTs | 2.05 | HS578T_cells, HMEC, BT474_cells |
| 108794 | AA129468 | Hs.203392 | ESTs | 2.04 | HS578T_cells, HMEC, A549_cells |
| 111896 | R38936 | Hs.24894 | H sapiens clone 25248 mRNA seq | 2.04 | HS578T_cells, PC3_cells, 293T_cells |
| 101849 | M94167 | Hs.172816 | neuregulin 1 | 2.04 | HMEC, HS578T_cells, HMEC (total RNA) |
| 119913 | W85931 | Hs.58785 | ESTs | 2.04 | HMEC, BT474_cells, MB231_cells |
| 130785 | AA242826 | Hs.19405 | caspase recruitment domain 4 | 2.04 | HMEC, HS578T_cells, BT474_cells |
| 124702 | R06984 | Hs.7745 | ESTs; Weakly similar to TESTIS-SPECIFIC | 2.03 | Fibroblasts 2, PRSC_con, HMEC |
| 105769 | AA478001 | Hs.225935 | diacylglycerol O-acyltransferase (mouse) | 2.03 | PC3_cells, EB_cells, HS578T_cells |
| 132219 | N48682 | Hs.172971 | ESTs | 2.03 | HT29_cells, PC3_cells, A549_cells |
| 122033 | AA431334 | Hs.109297 | ESTs | 2.03 | OVCAR_cells, A549_cells, Caco2 |
| 120461 | AA251301 | | zs10b02.s1 NCI_CGAP_GCB1 H sapiens cDNA | 2.03 | HS578T_cells, EB_cells, EB_cells |
| | | | contains Alu repetitive element;, mRNA | | |
| 134959 | U90550 | Hs.91813 | butyrophilin; subfamily 2; member A2 | 2.03 | HMEC, Fibroblasts 2, EB_cells |
| 104909 | AA055892 | Hs.14543 | ESTs | 2.03 | Lu_SC_H345, PC3_cells, DU145_cells |
| 101950 | S79219 | Hs.80741 | propionyl Coenzyme A carboxylase; alpha | 2.03 | Lu_SC_H69, EB_cells, CALU6_cells |
| 133878 | D78947 | Hs.7718 | ESTs; Weakly similar to weak similarity | 2.02 | EB_cells, MCF7, MB231_cells |
| 103459 | X99894 | Hs.32938 | insulin promoter factor 1; homeodomain t | 2.02 | EB_cells, Lu_AD_H23, Lu_AD_358 |
| 125507 | AI436377 | Hs.258590 | tetraspanin TM4-B | 2.02 | A549_cells, Lu_SQ_H520, Lu_AD_H23 |
| 116857 | F04014 | Hs.65996 | ESTs | 2.01 | HS578T_cells, HMEC, MB231_cells |
| 112920 | T10234 | Hs.4275 | ESTs | 2.01 | HS578T_cells, EB_cells, PRSC_con |
| 105533 | AA258572 | Hs.6418 | ESTs; Moderately similar to seven transm | 2.01 | HS578T_cells, HMEC, EB_cells |
| 126762 | AA064671 | | zm13b04.r1 Stratagene pancreas (#937208) | 2.01 | RPWE_2, Lu_AD_H23, Lu_AD_358 |
| | | | similar to TR:G413842 G413842 NONCLASSI | | |
| 128999 | R37808 | Hs.107765 | ESTs | 2.01 | HS578T_cells, OVCAR_cells, EB_cells |
| 133902 | AA114858 | Hs.7745 | ESTs; Weakly similar to TESTIS-SPECIFIC | 2 | Fibrablasts 2, PRSC_con, DU145_cells |

TABLE 2

| | |
|---|---|
| Pkey: | Unique Eos probeset identifier number |
| ExAccn: | Exemplar Accession number, Genbank accession number |
| UnigeneID: | Unigene number |
| Unigene Title: | Unigene gene title |

| Pkey | Ex Accn | UniG_ID | Complete_Title | Ratio Mets/BS | Top 3 expressing cell lines |
|---|---|---|---|---|---|
| 101447 | M21305 | Hs.247946 | Human alpha satellite and satellite 3 ju | 110.98 | EB_cells, Fibroblasts2, A549_cells |
| 105039 | AA130349 | Hs.36475 | ESTs | 9.13 | EB_cells, OVCAR_cells, Lu_SC_H345 |
| 106094 | AA419461 | Hs.18127 | ESTs | 8.51 | H729_cells, MB-MDA-453, HS578T_cells |
| 105777 | AA348412 | Hs.23096 | ESTs | 8.4 | 293T_cells, OVCAR_cells, EB_cells |
| 129818 | N54841 | Hs.172572 | ESTs | 7.2 | Lu_SC_H69, EB_cells, Lu_SC_H345 |
| 118475 | N66845 | Hs.165411 | ESTs; Weakly similar to !!!! ALU CLASS B | 7 | DU145_cells, EB₁₃ cells, Caco2 |
| 112170 | R48744 | Hs.192878 | ESTs | 6.91 | 293T_cells, DU145_cells, HT29_cells |
| 114918 | AA236813 | Hs.72324 | ESTs; Highly simharto unknown [H.sapie | 6.6 | EB_cells, 293T_cells, DU145_cells |
| 104590 | R79750 | Hs.83623 | nuclear receptor subfamily 1; group I; m | 6.58 | 293T_cells, OVCAR_cells, HMEC |
| 120625 | AA285053 | Hs.107168 | ESTs | 6.55 | CALU6_cells, OVCAR_cells, EB_cells |
| 115650 | AA404564 | Hs.47094 | ESTs | 6.43 | EB_cells, LNCaP_cells, Lu_SC_H345 |
| 124568 | N67086 | Hs.102000 | ESTs | 6.35 | PC3_cells, A549_cells, DU145_cells |
| 134238 | R81509 | Hs.184571 | splicing factor arginine/serine-rich 11 | 6.32 | 293T_cells, Lu_SC_H345, HMEC |
| 114721 | AA131450 | Hs.103822 | ESTs | 6.13 | Caco2, MB-MDA-435s, PRSC_log |
| 106145 | AA424791 | Hs.5734 | KIAA0679 protein | 6 | OVCAR_cells, EB_cells, 293T_cells |
| 114610 | AA081079 | | zn32h9.s1 Stratagene endothelial cell 93 | 5.97 | PRSC_con, DU145_cells, HS578T_cells |
| | | | IMAGE:549185 3′, mRNA seq | | |
| 130281 | R12777 | Hs.15395 | ESTs; Weakly similar to ARGINYL-TRNA SYN | 5.94 | PRSC_con, HT29_cells, EB_cells |
| 124690 | R05818 | Hs.173830 | ESTs | 5.92 | LNCaP_cells, EB_cells, OVCAR_cells |
| 113490 | T88700 | Hs.173374 | ESTs | 5.81 | DU145_cells, PC3_cells, HMEC (total RNA) |
| 104425 | H88496 | Hs.40583 | ESTs | 5.77 | OVCAR_cells, HS578T_cells, A549_cells |
| 118828 | N79496 | Hs.50824 | EST | 5.45 | LNCaP_cells, OVCAR_cells, DU145_cells |
| 129076 | AA262179 | Hs.169343 | ESTs | 5.35 | 293T_cells, BT474_cells, MCF7 |
| 109684 | F09317 | Hs.140885 | ESTs; Weakly similar to LINE-1 REVERSE T | 5.34 | Fibroblasts 2, Lu_SC_H69, DU145_cells |
| 104558 | R56678 | Hs.88959 | Human DNA seq from clone 967N21 on chr 2 | 5.32 | EB_cells, PC3_cells, Lu_SC_H345 |
| | | | part of KIAA0172; the gene for a novel | | |
| 109032 | AA158234 | Hs.72222 | ESTs | 5.23 | HT29_cells, PC3_cells, Lu_AD_358 |
| 129350 | U50535 | Hs.110630 | Human BRCA2 region: mRNA seq CG006 | 5.2 | 293T_cells, EB_cells, DU145_cells |
| 112662 | R85436 | Hs.193150 | ESTs | 5.2 | MB-MDA-435s, PRSC_con, MB-MDA-453 |
| 132902 | AA490969 | Hs.168147 | ESTs | 5.18 | PC3_cells, LNCaP_cells, CALU6_cells |
| 126872 | AA136653 | | ESTs | 5.04 | EB_cells, Fibroblasts 2, A549_cells |
| 122528 | AA449804 | Hs.250992 | EST | 5.04 | Lu_SC_H345, PRSC_con, LNCaP_cells |
| 102193 | U20758 | Hs.313 | secreted phosphoprotein 1 (osteopontin; | 5.02 | Lu_LC_H460, A549_cells, MB-MDA-435s |
| 121332 | AA404384 | Hs.97921 | ESTs | 5.01 | EB₁₃ cells, Lu_SC_H69, DU145_cells |
| 135357 | AA235803 | Hs.79572 | cathepsin D (lysosomal aspartyl protease | 4.96 | EB_cells, MCF7, DU145_cells |
| 109141 | AA176428 | Hs.193380 | ESTs | 4.86 | DU145_cells, PC3_cells, PRSC_log |
| 135324 | AA082041 | Hs.9873 | ESTs | 4.83 | EB_cells, Lu_SC_H345, HS578T_cells |
| 124875 | R70506 | Hs.207693 | ESTs; Weakly similar to !!!! ALU SUBFAMI | 4.75 | DU145_cells, OVCAR_cells, LNCaP_cells |
| 102380 | U40434 | Hs.155981 | mesothelin | 4.71 | OVCAR_cells, Lu_AD_H23, RPWE_2 |
| 127956 | AA826117 | Hs.194013 | ESTs | 4.69 | EB_cells, HS578T_cells, DU145_cells |
| 125038 | T78089 | Hs.168887 | ESTs | 4.58 | OVCAR_cells, 293T_cells, DU145_cells |
| 102515 | U52696 | | Humn adrenal Creb-rp hmlg (Creb-rp), com | 4.57 | Lu_SC_H345, Lu_SC_H69, HT29_cells |
| 109027 | AA157818 | Hs.238380 | Human endogenous retroviral protease mRN | 4.57 | PC3_cells, EB_cells, Lu_SQ_H520 |
| 115096 | AA255991 | Hs.175319 | ESTs | 4.57 | OVCAR_cells, 293T_cells, PC3_cells |
| 123470 | AA599106 | Hs.194208 | ESTs | 4.55 | LNCaP_cells, Lu_SC_H69, 293T_cells |
| 113219 | T59257 | Hs.194407 | ESTs | 4.55 | A549_cells, 293T_cells, 293T_cells |
| 123433 | AA598661 | Hs.112478 | ESTs | 4.55 | EB_cells, OVCAR_cells, HT29_cells |
| 135182 | M28170 | Hs.96023 | CD19 antigen | 4.53 | OVCAR_cells, DU145_cells, EB_cells |
| 121721 | AA419470 | Hs.199961 | ESTs | 4.51 | DU145_cells, LNCaP_cells, EB_cells |
| 129126 | H88486 | Hs.108806 | ESTs | 4.45 | LNCaP_cells, Caco2, EB_cells |
| 135232 | AA342457 | Hs.96800 | ESTs; Moderately similar to !!!! ALU SUB | 4.43 | LNCaP_cells, DU145_cells, OVCAR_cells |
| 124847 | R60044 | Hs.106706 | ESTs; Highly similar to BETA-CATENIN [H. | 4.42 | OVCAR_cells, CALU6_cells, CALU6_cells |
| 110349 | H40988 | | ESTs; Weakly similar to !!!! ALU SUBFAMI | 4.39 | DU145_cells, OVCAR_cells, LNCaP_cells |
| 134402 | U25165 | Hs.82712 | fragile X mental retardation; autosomal | 4.38 | HS578T_cells, OVCAR_cells, DU145_cells |
| 115494 | AA290603 | Hs.256517 | ESTs | 4.36 | Lu_SC_H345, OVCAR_cells, PC3_cells |
| 119174 | R71234 | | yi54c08.s1 Soares placenta Nb2HP H sapie | 4.33 | DU145_cells, OVCAR_cells, LNCaP_cells |
| | | | transcript (rRNA); gb:541458 ROD CGMP- | | |
| | | | BETA-SUBUNIT (HUMAN); contain | | |
| 121943 | AA429265 | Hs.126759 | ESTs | 4.3 | EB_cells, HT29_cells, Lu_SC_H69 |
| 110856 | N33063 | Hs.23291 | ESTs; Weakly similar to S164 [H.sapiens] | 4.28 | OVCAR_cells, EB_cells, Lu_SC_H69 |
| 102474 | U49973 | | Human Tigger1 transposable element comp | 4.28 | DU145_cells, LNCaP_cells, OVCAR_cells |
| 123458 | AA598963 | Hs.112499 | KIAA0612 protein | 4.27 | A549_cells, A549_cells, BT474_cells |
| 116459 | AA621399 | Hs.64193 | ESTs | 4.22 | Caco2, HS578T_cells, MB-MDA-435s |
| 126301 | N62371 | Hs.100043 | ESTs; Weakly similar to Similar to cutic | 4.22 | PC3_cells, DU145_cells, Lu_SC_H345 |
| 123461 | AA598990 | Hs.251119 | EST | 4.22 | Lu_SC_H345, Lu_SC_H69, OVCAR_cells |
| 130588 | AA287735 | Hs.16411 | Human DNA seq from done 1189B24 on chro | 4.2 | EB_cells, LNCaP_cells, MCF7 |
| | | | MLRQ subunit (EC 1.6.5.3; EC 1.6.99.3; | | |
| | | | Tyrosine-protein Kinase FER (EC 2.7.1.1 | | |
| 125756 | W25498 | Hs.81634 | ATP synthase; H + transporting; mitochond | 4.2 | HMEC, EB_cells, DU145_cells |
| 135009 | AA040507 | Hs.251865 | ESTs | 4.19 | 293T_cells, EB_cells, DU145_cells |
| 107001 | AA598589 | Hs.24492 | ESTs | 4.18 | 293T_cells, DU145_cells, EB_cells |
| 124896 | R82063 | Hs.101594 | EST | 4.16 | OVCAR_cells, Lu_SC_H345, HMEC (total RNA) |
| 119404 | 792950 | | ye27c10.s1 Stratagene lung (#937210) H s | 4.15 | DU145_cells, PC3_cells, Fibroblasts 2 |
| 125090 | T91518 | | ye20f05.s1 Stratagene lung (#937210) H s | 4.14 | LNCaP_cells, DU145_cells, OVCAR_cells |
| | | | contains Alu repetitive element; contain | | |
| 117348 | N24157 | Hs.139615 | ESTs | 4.1 | Lu_SC_H345, Lu_SC_H69, PRSC_log |
| 111389 | N95837 | Hs.169111 | ESTs; Weakly similar to LB2A [D.melanoga | 4.1 | DU145_cells, MCF7, LNCaP_cells |
| 134977 | AA464698 | Hs.19390 | ESTs; Weakly similar to bullous pemphigo | 4.09 | OVCAR_cells, Fibroblasts 2, Lu_SC_H69 |
| 124696 | R06273 | Hs.186467 | ESTs; Moderately similar to !!!! ALU SUB | 4.09 | OVCAR_cells, Lu_SC_H345, PRSC_con |
| 124090 | H09570 | Hs.143032 | ESTs; Weakly similar to neuronal thread | 3.98 | DU145_cells, OVCAR_cells, Lu_SC_H345 |
| 133992 | R46354 | Hs.169832 | zinc finger protein 42 (myeloid-speciflc | 3.98 | HT29_cells, MB231_cells, BT474_cells |
| 126009 | H51652 | Hs.242985 | hemoglobin; gamma G | 3.96 | Lu_SC_H69, OVCAR_cells, EB_cells |
| 114161 | Z38904 | Hs.22385 | ESTs; Weakly similar to KIAA0970 protein | 3.94 | HS578T_cells, EB_cells, PRSC_con |
| 109171 | AA180356 | Hs.73700 | EST | 3.94 | 293T_cells, MB-MDA-435s, A549_cells |
| 122007 | AA430629 | Hs.98564 | ESTs | 3.93 | PC3_cells, A549_cells, OVCAR_cells |
| 131936 | AA094865 | Hs.179972 | interferon; alpha-inducible protein (clo | 3.9 | CALU6_cells, EB_cells, Lu_SC_H69 |
| 128668 | AA194849 | Hs.103422 | ESTs | 3.9 | Lu_AD_H23, EB_cells, Lu_SC_H69 |
| 124977 | T33859 | Hs.190452 | KIAA0365 gene product | 3.89 | 293T_cells, DU145_cells, EB_cells |
| 107048 | AA600012 | Hs.10669 | ESTs; Moderately similarto KIAA0400 [H. | 3.89 | PC3_cells, HS578T_cells, DU145_cells |
| 105358 | AA236034 | Hs.25362 | ESTs | 3.89 | Caco2, EB_cells, CALU6_cells |
| 135106 | AA599037 | Hs.9456 | SWI/SNF related; matrix assocd; actin de | 3.86 | EB_cells, LNCaP_cells, Caco2 |
| 106686 | AA463215 | Hs.29896 | ESTs; Weakly similar to proline-rich pro | 3.85 | OVCAR_cells, DU145_cells, EB_cells |
| 132093 | AA400091 | Hs.39421 | ESTs | 3.85 | OVCAR_cells, OVCAR_cells, LNCaP_cells |
| 128651 | AA446990 | Hs.103135 | ESTs | 3.84 | EB_cells, LNCaP_cells, OVCAR_cells |
| 102459 | U48936 | | Human amiloride-sensitive epithelial sod | 3.84 | HT29_cells, BT474_cells, Lu_SC_H69 |
| 113732 | 798288 | Hs.193295 | ESTs; Weakly similar to !!!! ALU SUBFAMI | 3.82 | DU145_cells, OVCAR_cells, LNCaP_cells |
| 116000 | AA448710 | Hs.41327 | ESTs | 3.82 | DU145_cells, MB-MDA-453, Lu_SC_H69 |
| 120748 | AA303153 | Hs.237994 | EST; Weakly similarto !!!! ALU SUBFAMIL | 3.82 | DU145_cells, DU145_cells, Lu_SC_H345 |
| 116318 | AA490830 | Hs.58570 | deleted in cancer 1; RNA helicase HDB/DI | 3.79 | MB-MDA-453, CALU6_cells, EB_cells |
| 114366 | Z41747 | Hs.469 | succinate dehydrogenase complex; subunit | 3.78 | DU145_cells, Fibroblasts 2, Caco2 |
| 107248 | D59894 | Hs.34782 | ESTs | 3.75 | LNCaP_cells, DU145_cells, EB_cells |
| 132713 | AA286906 | Hs.55335 | ESTs | 3.75 | OVCAR_cells, EB_cells, Lu_SC_H345 |
| 102222 | U24683 | Hs.159386 | Immunoglobulin heavy variable 4-4 | 3.73 | EB_cells, OVCAR_cells, 293T_cells |
| 108201 | AA057518 | Hs.63394 | ESTs | 3.72 | 293T_cells, DU145_cells, EB_cells |
| 119940 | W86779 | Hs.171807 | DKFZP586B0319 protein | 3.71 | EB_cells, Caco2, DU145_cells |
| 106508 | AA452590 | Hs.30348 | ESTs | 3.67 | EB_cells, LNCaP_cells, 293T_cells |
| 114360 | Z41592 | Hs.22129 | hypothetical protein | 3.67 | HT29_cells, Lu_SQ_H520, Lu_SQ_H520 |
| 100991 | J03764 | Hs.82085 | plasminogen activator inhibitor; type I | 3.67 | Fibroblasts 2, HS578T_cells, MB231_cells |
| 107580 | AA002091 | Hs.175476 | ESTs; Weakly similar to !!!! ALU SUBFAMI | 3.67 | OVCAR_cells, LNCaP_cells, Lu_SC_H345 |
| 111685 | R21408 | Hs.106095 | ESTs | 3.66 | OVCAR_cells, A549_cells, 293T_cells |
| 128336 | AI242720 | Hs.146043 | ESTs; Weakly similar to alternatively sp | 3.66 | Lu_SC_H345, Caco2, OVCAR_cells |
| 130868 | AA004900 | Hs.171917 | ESTs; Weakly similar smlr to glycerophos | 3.61 | EB_cells, HS578T_cells, LNCaP_cells |
| 116802 | H44061 | Hs.194026 | ESTs | 3.6 | Lu_SC_H345, OVCAR_cells, DU145_cells |
| 130753 | Z46632 | Hs.189 | phosphodiesterase 40; cAMP-specific (dun | 3.6 | Lu_SC_H69, Lu_AD_H23, Lu_SC_H345 |
| 123074 | AA485117 | Hs.105653 | ESTs | 3.6 | 293T_cells, MB231_cells, Fibroblasts 2 |
| 114317 | Z41038 | Hs.469 | succinate dehydrogenase complex; subunit | 3.6 | DU145_cells, HS578T_cells, CALU6_cells |
| 134194 | AA233231 | Hs.79828 | ESTs | 3.59 | BT474_cells, MB231_cells, HT29_cells |
| 127752 | AA808388 | Hs.211167 | ESTs | 3.59 | Lu_SQ_H520, MB-MDA.435s, DU145_cells |
| 123526 | AA608657 | | ESTs; Moderately similar to !!!! ALU SUB | 3.59 | DU145_cells, OVCAR_cells, LNCaP_cells |
| 127917 | AA211895 | Hs.118831 | EST; Highly similar to dJ1163J1.2.1 [H.s | 3.58 | Lu_SC_H345, OVCAR_cells, PRSC_con |
| 105941 | AA404427 | Hs.10669 | ESTs; Moderately similar to KIAA0400 [H. | 3.58 | PC3_cells, DU145_cells, HS578T_cells |
| 124694 | R06108 | Hs.135258 | ESTs | 3.56 | Lu_AD_H23, Lu_SQ_H520, Lu_AD_358 |
| 105658 | AA282571 | Hs.203772 | FSHD region gene 1 | 3.56 | DU145_cells, EB_cells, A549_cells |
| 111168 | N66951 | Hs.238380 | Human endogenous retroviral protease mRN | 3.55 | PC3_cells, EB_cells, MB231_cells |
| 133254 | AA156670 | Hs.180780 | H sapiens agrin precursor mRNA; partial | 3.54 | OVCAR_cells, DU145_cells, PC3_cells |
| 132840 | U33821 | | Tax1 (human 7-cell leukemia virus type I | 3.53 | MB231_cells, CALU6_cells, BT474_cells |
| 116562 | D25807 | Hs.90145 | ESTs | 3.52 | MB231_cells, BT474_cells, Lu_SC_H345 |
| 126045 | N80361 | Hs.14248 | ESTs | 3.51 | DU145_cells, Lu_SC_H345, OVCAR_cells |
| 122878 | AA465341 | Hs.99640 | ESTs | 3.47 | HT29_cells, OVCAR_cells, HMEC |
| 105220 | AA210695 | Hs.17212 | ESTs | 3.47 | MB-MDA-435s, HT29_cells, HT29_cells |
| 127001 | AA731636 | Hs.59319 | ESTs; Weakly similar to !!!! ALU SUBFAMI | 3.45 | LNCaP_cells, DU145_cells, Lu_SC_H345 |
| 112693 | R88741 | Hs.91065 | ESTs; Moderately similar to proliferatio | 3.44 | EB_cells, LNCaP_cells, DU145_cells |
| 104935 | AA063280 | Hs.35552 | ESTs | 3.43 | LNCaP_cells, CALU6_cells, 293T_cells |
| 128710 | J04813 | Hs.104117 | cytochrome P450; subfamily IIIA (niphedi | 3.41 | HT29_cells, A549_cells, Fibroblasts 2 |
| 131996 | D86956 | Hs.36927 | heat shock 105 kD | 3.4 | EB_cells, PC3_cells, Lu_SC_H345 |
| 119229 | T03229 | | H sapiens (clone 104) retinoblastoma 1 g | 3.4 | DU145_cells, Lu_SC_H345, EB_cells |
| 128046 | AA873285 | Hs.137947 | ESTs | 3.39 | EB_cells, LNCaP_cells, DU145_cells |
| 105175 | AA186804 | Hs.25740 | ESTs; Weakly similar to ubiquitous TPR m | 3.39 | PC3_cells, MCF7, DU145_cells |
| 132349 | Y00705 | Hs.181286 | serine protease inhibitor; Kazal type 1 | 3.38 | Caco2, EB_cells, Lu_SC_H69 |
| 101569 | M32053 | | Human H19 RNA gene, complete cds | 3.37 | Lu_SC_H69, MCF7, OVCAR_cells |
| 116389 | AA599011 | | troponin T1; skeletal; slow | 3.36 | DU145_cells, LNCaP_cells, OVCAR_cells |
| 130641 | AA182001 | Hs.17155 | ESTs | 3.36 | DU145_cells, MB-MDA-435s, HS578T_cells |
| 109362 | AA214615 | Hs.194348 | ESTs | 3.33 | HT29_cells, Fibroblasts 2, BT474_cells |
| 106278 | AA432292 | Hs.23388 | ESTs; Moderately similar to !!!! ALU SUB | 3.33 | EB_cells, Fibroblasts 2, BT474_cells |
| 127241 | AA321849 | Hs.248340 | H sapiens mRNA; cDNA DKFZp564J2116 (from | 3.32 | LNCaP_cells, DU145_cells, EB_cells |
| 133339 | N64588 | Hs.71252 | ESTs | 3.32 | DU145_cells, EB_cells, Caco2 |
| 113260 | T64896 | Hs.237992 | ESTs | 3.32 | Lu_SQ_H345, LNCaP_cells, Lu_SC_H69 |
| 133349 | N75791 | Hs.7153 | L-3-hydroxyacyl-Coenzyme A dehydrogenase | 3.31 | Caco2, EB_cells, OVCAR_cells |
| 107149 | AA621159 | Hs.23284 | ESTs | 3.29 | HS578T_coells, DU145_cells, PRSC_con |
| 133195 | AA350744 | Hs.181409 | KIAA1007 protein | 3.29 | EB_cells, Lu_AD_H23, Lu_AD_358 |
| 111302 | N73838 | Hs.15049 | ESTs | 3.29 | DU145_cells, EB_cells, HS578T_cells |
| 106414 | AA447971 | Hs.28827 | ESTs | 3.28 | A549_cells, OVCAR_cells, PC3_cells |
| 121768 | AA421561 | Hs.251664 | insulin-like growth factor 2 (somatomedi | 3.28 | Caco2, PRSC_con, PRSC_log |
| 117176 | H98670 | Hs.49753 | ESTs; Weakly similar to hypothetical pro | 3.28 | PRSC_log, CALU6_cells, OVCAR_cells |
| 131320 | AA171948 | Hs.145696 | splicing factor(CC1.3) | 3.28 | EB_cells, LNCaP_cells, DU145_cells |
| 100700 | HG3227-H | | Guanine Nucleotide-Binding Protein Hsr1 | 3.27 | EB_cells, RPWE_2, Lu_AD_H23 |
| 134275 | AA132328 | Hs.3688 | acid-inducible phosphoprotein | 3.26 | EB_cells, DU145_cells, LNCaP_cells |
| 117667 | N39214 | Hs.44708 | Ser-Thr protein kinase related to the my | 3.26 | LNCaP_cells, DU145_cells, MB-MDA-453 |
| 124889 | R78604 | Hs.101570 | ESTs | 3.25 | Lu_AD_H23, Lu_SC_H69, Lu_SC_H345 |
| 126631 | W95117 | Hs.193337 | ESTs | 3.25 | Lu_SC_H345, OVCAR_cells, Lu_SC_H69 |
| 105643 | AA282069 | Hs.173802 | KIAA0603 gene product | 3.24 | Caco2, EB_cells, 293T_cells |
| 132718 | AA056731 | Hs.554 | Siogren syndrome antigen A2 (60 kD; ribon | 3.24 | CALU6_cells, OVCAR_cells, A549_cells |
| 116417 | AA609309 | Hs.239302 | ESTs; Weakly similar to !!!! ALU SUBFAMI | 3.24 | A549_cells, CALU6_cells, 293T_cells |
| 108039 | AA041341 | Hs.46670 | ESTs | 3.24 | 293T_cells, EB₁₃ cells, Caco2 |
| 114116 | Z38496 | Hs.103283 | KIAA0594 protein | 3.23 | DU145_cells, OVCAR_cells, EB_cells |
| 124514 | N58045 | Hs.142737 | ESTs | 3.22 | EB_cells, Caco2, Lu_SQ_H520 |
| 110802 | N26651 | Hs.252748 | ESTs | 3.22 | LNCaP_cells, MB-MDA-435s, MB-MDA-453 |
| 106920 | AA490899 | Hs.24462 | ESTs | 3.22 | DU145_cells, EB_cells, OVCAR_cells |
| 123523 | AA608588 | Hs.193634 | ESTs | 3.21 | DU145_cells, LNCaP_cells, OVCAR_cells |
| 131564 | AA491465 | Hs.28792 | ESTs | 3.2 | HS578T_cells, HMEC (total RNA), HMEC |
| 119423 | T99544 | Hs.173734 | ESTs; Weakly similar to !!!! ALU CLASS B | 3.2 | EB_cells, DU145_cells, Caco2 |
| 128736 | F03934 | Hs.104607 | ESTs | 3.19 | PC3_cells, Lu_SQ_H520, Lu_SC_H69 |
| 101511 | M27826 | Hs.238380 | Human endogenous retroviral protease mRN | 3.18 | PC3_cells, DU145_cells, Lu_SQ_H520 |
| 114509 | AA043551 | Hs.95249 | ESTs | 3.18 | EB₁₃ cells, Lu_SC_H345, DU145_cells |
| 124196 | H52617 | Hs.144167 | ESTs | 3.17 | BT474_cells, MB231_cells, HMEC |
| 129095 | L12350 | Hs.108623 | thrombospondin 2 | 3.17 | Fibroblasts 2, PRSC_con, PRSC_log |
| 116457 | AA621367 | Hs.119683 | ESTs | 3.17 | 293T_cells, Lu_SC_H345, CALU6_cells |
| 117040 | H89112 | | yw25e5.s1 Morton Fetal Cochlea H sapiens | 3.16 | OVCAR_cells, 293T_cells, EB_cells |
| 129112 | N32521 | Hs.108738 | ESTs | 3.16 | EB_cells, Fibroblasts 2, MB231_cells |
| 130418 | J03242 | Hs.251664 | insulin-like growth factor 2 (somatomedi | 3.16 | Caco2, PRSC_con, PRSC_log |
| 131199 | R80048 | Hs.234433 | ESTs; Weakly similar to transporter prot | 3.15 | PC3_cells, EB_cells, OVCAR_cells |
| 110357 | H41529 | Hs.33549 | ESTs; Highly similar to sulfonylurea rec | 3.15 | Lu_SQ_H345, PRSC_con, Lu_AD_H23 |
| 130068 | AA608903 | Hs.106220 | KIAA0336 gene product | 3.15 | OVCAR_cells, CALU6_cells, HS578T_cells |
| 127423 | T47546 | Hs.119252 | tumor protein; translationally-controlle | 3.15 | EB_cells, PRSC_con, LNCaP_cells |
| 105028 | AA126719 | Hs.25282 | ESTs | 3.14 | LNCaP_cells, PC3_cells, EB_cells |
| 102349 | U37547 | Hs.75263 | apoptosis inhibitor 1 | 3.14 | DU145_cells, HS578T_cells, LNCaP_cells |
| 105126 | AA157814 | Hs.36288 | ESTs | 3.13 | EB_cells, HS578T_cells, LNCaP_cells |
| 115465 | AA286941 | Hs.43691 | ESTs | 3.12 | EB_cells, DU145_cells, 293T_cells |
| 133240 | AA086452 | Hs.68731 | triadin | 3.12 | Lu_SQ_H520, Lu_AD_H23, PRSC_log |
| 122698 | AA456112 | Hs.99410 | ESTs | 3.12 | DU145_cells, OVCAR_cells, A549_cells |
| 123553 | AA608841 | Hs.111977 | ESTs | 3.12 | EB_cells, Caco2, DU145_cells |
| 133437 | R57419 | Hs.7370 | ESTs | 3.11 | HS578T_cells, 293T_cells, Caco2 |
| 104956 | AA074880 | Hs.120975 | ESTs; Weakly similar to hypothetical pro | 3.11 | OVCAR_cells, Fibroblasts 2, Caco2 |
| 116314 | AA490588 | Hs.43118 | ESTs | 3.11 | EB_cells, MB-MDA-435s, HT29_cells |
| 120562 | AA280036 | Hs.173912 | eukaryotic translation initiation factor | 3.11 | LNCaP_cells, DU145_cells, EB_cells |
| 108770 | AA127845 | Hs.71027 | EST | 3.11 | Lu_LC_H460, Lu_SC_H345, Lu_AD_358 |
| 129791 | F02778 | Hs.173887 | KIAA0876 protein | 3.1 | Lu_SC_H345, Lu_SC_H69, PRSC_log |
| 115783 | AA424487 | Hs.72289 | ESTs; Weakly similar to LIV-1 protein [H | 3.09 | Lu_AD_358, EB₁₃ cells, PC3_cells |
| 107630 | AA007218 | Hs.60178 | ESTs | 3.07 | Lu_SC_H345, CALU6_cells, Lu_SC_H69 |
| 124339 | H99093 | Hs.6179 | H sapiens mRNA; cDNA DKFZp586K2322 (from | 3.07 | 293T_cells, MB-MDA-453, Caco2 |
| 122314 | AA442257 | Hs.192076 | ESTs | 3.07 | 293T_cells, LNCaP_cells, PC3_cells |
| 104589 | R79299 | Hs.241160 | ESTs; Moderately similarto !!!! ALU SUB | 3.07 | 293T_cells, DU145_cells, EB_cells |
| 115687 | AA410508 | Hs.163765 | ESTs; Moderately smlr to ORF derived frm | 3.06 | Caco2, EB_cells, MB231_cells |
| 123796 | AA620390 | Hs.247444 | ESTs | 3.06 | Lu_SC_H345, LNCaP_cells, DU145_cells |
| 106483 | AA451676 | Hs.30299 | IGF-II mRNA-binding protein 2 | 3.06 | OVCAR_cells, HMEC (total RNA), HMEC |
| 133318 | AA256168 | Hs.70838 | ESTs | 3.05 | OVCAR_cells, LNCaP_cells, 293T_cells |
| 117244 | N20979 | Hs.1757 | L1 cell adhesion molecule (hydrocephalus | 3.05 | MB_cells, MCF7, CALU6_cells |
| | | | thumbs) syndrome; spastic paraplegia 1) | | |
| 130797 | AA430050 | Hs.180948 | KIAA0729 protein | 3.05 | EB_cells, DU145_cells, DU145_cells |
| 128959 | D79791 | Hs.107381 | ESTs; Weakly similar to F38A5.1 [C.elega | 3.05 | LNCaP_cells, HS578T_cells, Lu_SQ_H520 |
| 120481 | AA252703 | Hs.191754 | ESTs | 3.04 | EB_cells, Fibroblasts 2, PRSC_con |
| 126649 | AA856990 | Hs.125058 | ESTs | 3.03 | OVCAR_cells, LNCaP_cells, 293T_cells |
| 106970 | AA504835 | Hs.24252 | ESTs | 3.03 | EB_cells, OVCAR_cells, 293T_cells |
| 126488 | N34935 | Hs.25633 | ESts; Highly similar to ARF GTPase-activ | 3.03 | LU_AD_358, MCF7, MB231_cells |
| 119498 | W37226 | Hs.55573 | ESts | 3.01 | 293T_cells, HS578T_cells, CALU6_cells |
| 129967 | H99653 | Hs.138618 | ESTs | 3.01 | Lu_SC_H345, Lu_SC_H69, PRSC_log |
| 130698 | AA037357 | Hs.188212 | Ests | 3.01 | OVCAR_cells, LNCaP_cells, DU145_cells |
| 111018 | N54067 | Hs.3626 | mitogen-activated protein kinase kinase | 3.01 | PC3_cells, Caco2, Fibroblasts 2 |
| 123196 | AA489250 | Hs.59403 | serine palmitoyltransferase; subunit II | 3 | Lu_SC_H345, BT474_cells, Lu_SC_H69 |
| 133229 | AA203433 | Hs.6834 | KIAA1014 protein | 3 | OVCAR_cells, 293T_cells, EB_cells |
| 130405 | H88359 | Hs.155396 | nuclear factor (erythroid-derived 2)-lik | 3 | PRSC_con, EB₁₃ cells, DU145_cells |
| 107881 | AA025567 | Hs.61273 | H sapiens chromosome 19; cosmid R32611 | 3 | Lu_SQ_H520, MCF7, Lu_AD_358 |
| 116589 | D59570 | Hs.17132 | ESTs | 3 | EB_cells, A549_cells, HS578T_cells |
| 105479 | AA255546 | Hs.23467 | ESTs | 2.99 | Lu_SC_H345, PC3_cells, OVCAR_cells |
| 115560 | AA393812 | Hs.50575 | ESTs; Moderately similar to !!!! ALU SUB | 2.99 | EB_cells, Lu_SC_H69, Fibroblasts 2 |
| 130166 | AA350690 | Hs.151411 | KIAA0916 protein | 2.98 | LNCaP_cells, EB_cells, 293T_cells |
| 123355 | AA504773 | Hs.160557 | ESTs | 2.98 | PRSC_con, PRSC_log, PRSC_log |
| 109546 | F01449 | Hs.26954 | ESTs | 2.97 | Lu_SC_H345, HT29_cells, BT474_cells |
| 129001 | AA448946 | Hs.107812 | ESTs; Weakly similar to proline-rich pro | 2.97 | EB_cells, Lu_AD_H23, Lu_AD_358 |
| 102259 | U28369 | Hs.82222 | sema domain; immunoglobulin domain (Ig); | 2.97 | EB_cells, MB231_cells, OVCAR_cells |
| 105583 | AA278907 | Hs.24549 | ESTs | 2.96 | EB_cells, DU145_cells, 293T_cells |
| 131859 | M90657 | Hs.3337 | transmembrane 4 superfamily member 1 | 2.96 | A549_cells, PC3_cells, DU145_cells |
| 114533 | AA053401 | Hs.177526 | ESTs | 2.96 | 293T_cells, Lu_LC_H460, PC3_cells |
| 110220 | H23543 | Hs.27090 | ESTs | 2.95 | PRSC_log, Lu_SC_H345, MB231_cells |
| 124917 | R91241 | Hs.75470 | hypothetical protein; expressed in osteo | 2.95 | Lu_SC_H345, Lu_SC_H69, PRSC_log |
| 127111 | AA805726 | Hs.220509 | ESTs | 2.94 | HS578T_cells, 293T_cells, 293T_cells |
| 134882 | N73762 | Hs.90638 | ESTs | 2.94 | EB_cells, MB-MDA-453, Fibroblasts 2 |
| 121788 | AA423968 | Hs.178113 | ESTs; Moderately similar to kinesin like | 2.94 | HT29_cells, CALU6_cells, HMEC |
| 128530 | AA504343 | Hs.183475 | H sapiens clone 25061 mRNA seq | 2.94 | DU145_cells, Lu_SC_H345, Caco2 |
| 128435 | AI301201 | Hs.147112 | ESTs | 2.93 | EB_cells, Lu_SQ_H520, PRSC_con |
| 113782 | W15580 | Hs.15342 | phosphate cytidylyltransferase 1; cholin | 2.93 | EB_cells, Lu_AD_H23, PRSC_log |
| 127569 | AA588536 | Hs.191783 | ESTs | 2.93 | EB_cells, HS578T_cells, Lu_AD_358 |
| 109642 | F04465 | Hs.22394 | ESTs; Weakly similar to weak similarity | 2.92 | PC3_cells, EB_cells, OVCAR_cells |
| | | | protein US)1 [C.elegans] | | |
| 114615 | AA083812 | Hs.159456 | DKFZP566F123 protein | 2.92 | A549_cells, HS578T_cells, PRSC_con |
| 126808 | AA086320 | | zn52d12.s1 Stratagene muscle 937209 H sa | 2.92 | Lu_SC_H69, Lu_SC_H345, EB_cells |
| 113947 | W84768 | Hs.141742 | ESTs | 2.92 | DU145_cells, Fibroblasts 2, MCF7 |
| 129455 | W27301 | Hs.187991 | DKFZPS64A122 protein | 2.91 | OVCAR_cells, DU145_cells, CALU6_cells |
| 107772 | AA018587 | Hs.40515 | ESTs; Weakly similar to !!!! ALU SUBFAMI | 2.91 | OVCAR_cells, EB_cells, PC3_cells |
| 127159 | AA284097 | Hs.237955 | RAB7; member RAS oncogene family | 2.91 | 293T_cells, OVCAR_cells, PC3_cells |
| 124792 | R44357 | Hs.132784 | ESTs; Weakly similar to cDNA EST EMBL:T0 | 2.91 | DU145_cells, DU145_cells, CALU6_cells |
| 109751 | F10210 | Hs.6679 | H sapiens mRNA; cDNA DKFZp586A0424 (from | 2.91 | EB_cells, Lu_SC_H69, 293T_cells |
| 128926 | AA481403 | Hs.107213 | ESTs; Highly similar to NY-REN-6 antigen | 2.9 | CALU6_cells, EB_cells, OVCAR_cells |
| 106637 | AA459961 | Hs.250824 | ESTs | 2.9 | EB_cells, Caco2, MB-MDA-435s |
| 132164 | U84573 | Hs.41270 | procollagen-lysine; 2-oxoglutarate 5-dio | 2.9 | DU145_cells, HS578T_cells, A549_cells |
| 128099 | AA905327 | | ESTs | 2.9 | MCF7, HMEC (total RNA), 293T_cells |
| 104818 | AA034947 | Hs.24831 | ESTs | 2.9 | EB_cells, Lu_LC_H460, 293T_cells |
| 126050 | H27267 | Hs.75860 | hydroxyacyl-Coenzyme A dehydrogenase/3-k | 2.89 | LNCaP_cells, DU145_cells, OVCAR_cells |
| | | | -Coenzyme A hydratase (trifunctional pro | | |
| 116696 | F09780 | Hs.66124 | EST | 2.89 | CALU6_cells, 293T_cells, 293T_cells |
| 135204 | AA421146 | Hs.183418 | cell division cycle 2-like 1 (PITSLRE pr | 2.89 | PC3_cells, EB_cells, LNCaP_cells |
| 134946 | AA406534 | Hs.193053 | ESTs; Weakly similar to hiwi [H.sapiens] | 2.88 | EB_cells, LNCaP_cells, Caco2 |
| 114975 | AA250850 | Hs.13944 | adrenergic; beta; receptor kinase 2 | 2.88 | EB_cells, EB_cells, EB_cells |
| 113792 | W35212 | Hs.17691 | ESTs; Weakly similar to env protein [H.s | 2.88 | MB-MDA-435s, Lu_SC_H69, CALU6_cells |
| 102322 | U34962 | Hs.54473 | cardiac-specific homeo box | 2.88 | 293T_cells, HT29_cells, Lu_AD_H23 |
| 125642 | AI096849 | Hs.25274 | ESTs; Moderately similar to putative sev | 2.88 | PC3_cells, CALU6_cells, 293T_cells |
| 100288 | D43951 | Hs.153834 | Human mRNA for KIAA0099 gene; complete c | 2.88 | 293T_cells, LNCaP_cells, EB_cells |
| 105878 | AA400184 | Hs.24656 | KIAA0907 protein | 2.88 | OVCAR_cells, DU145_cells, 293T_cells |
| 125262 | W88755 | Hs.108514 | ESTs; Highly similar to Trio [H.sapiens] | 2.88 | DU145_cells, HS578T_cells, MB231_cells |
| 114419 | AA011448 | Hs.106532 | ESTs; Weakly similar to transposon LRE2 | 2.88 | EB_cells, Lu_AD_H23, Fibroblasts 2 |
| 130639 | D59711 | Hs.17132 | ESTs | 2.87 | EB_cells, A549_cells, OVCAR_cells |
| 130972 | AA370302 | Hs.21739 | H sapiens mRNA; cDNA DKFZp586I1518 (from | 2.87 | 293T_cells, A549_cells, Lu_LC_H460 |
| 126906 | H66949 | Hs.168069 | ESTs; Highly similar to CALCIUM-BINDING | 2.87 | Lu_SC_H345, Lu_SC_H69, LNCaP_cells |
| 121807 | AA424507 | Hs.247478 | H sapiens Mut S homolog 5 gene; partial | 2.87 | Lu_SC_H69, HT29_cells, RPWE_2 |
| | | | 1C7; LST-1; lymphotoxin beta; tumor necr | | |
| 105474 | AA255440 | Hs.219614 | F-box protein FBL11 | 2.87 | Lu_AD_H23, Caco2, EB_cells |
| 122348 | AA443695 | Hs.231476 | ESTs | 2.87 | HT29_cells, Lu_SC_H69, BT474_cells |
| 116368 | AA521186 | Hs.94217 | ESTs | 2.86 | MB-MDA-453, OVCAR_cells, Lu_SC_H69 |
| 135143 | AA102644 | Hs.69559 | KIAA1096 protein | 2.86 | PC3_cells, EB_cells, 293T_cells |
| 106711 | AA464741 | Hs.143187 | Human DNA from chromosome 19-specific co | 2.86 | EB_cells, Lu_AD_H23, Lu_LC_H460 |
| 128583 | L32832 | Hs.101842 | AT-binding transcription factor 1 | 2.85 | LNCaP_cells, Caco2, EB_cells |
| 132139 | AA213410 | Hs.111554 | ADP-ribosylation factor-like 7 | 2.85 | A549_cells, HS578T_cells, Caco2 |
| 114484 | AA034378 | Hs.252351 | HERV-H LTR-associating 2 | 2.85 | PC3_cells, Lu_SQ_H520, MB231_cells |
| 124620 | N74051 | Hs.194092 | ESTs; Weakly similar to !!!! ALU SUBFAMI | 2.85 | Lu_SC_H345, MB231_cells, Fibroblasts 2 |
| 100403 | D85527 | | H sapiens mRNA for LIM domain, partial c | 2.84 | Lu_AD_358, Lu_AD_358, MB231_cells |
| 129795 | AA448627 | Hs.125163 | ESTs; Weakly similar to !!!! ALU SUBFAMI | 2.84 | Lu_SC_H345, OVCAR_cells, PC3_cells |
| 128258 | T70214 | Hs.183548 | ESTs | 2.84 | DU145_cells, DU145_cells, OVCAR_cells |
| 102662 | U70321 | Hs.130227 | tumor necrosis factor receptor superfami | 2.84 | EB_cells, Lu_AD_H23, Fibroblasts 2 |
| 132232 | AA252030 | Hs.42640 | ESTs | 2.84 | EB_cells, OVCAR_cells, Lu_SC_H345 |
| 106111 | AA421638 | Hs.6451 | ESTs | 2.83 | EB_cells, Lu_LC_H460, OVCAR_cells |
| 123963 | C13961 | Hs.210115 | EST | 2.83 | DU145_cells, LNCaP_cells, Lu_SC_H345 |
| 122783 | AA459895 | Hs.98988 | ESTs | 2.83 | EB_cells, MCF7, Lu_SC_H69 |
| 112788 | R96586 | Hs.163630 | ESTs | 2.82 | DU145_cells, Lu_SC_H345, EB_cells |
| 120823 | AA347546 | Hs.185780 | ESTs | 2.82 | HT29_cells, HMEC (total RNA), BT474_cells |
| 100378 | D80009 | Hs.10848 | KIAA0187 gene product | 2.82 | Caco2, PC3_cells, OVCAR_cells |
| 114677 | AA114163 | Hs.188877 | ESTs | 2.81 | DU145_cells, MCF7, EB_cells |
| 108085 | AA045602 | Hs.62863 | ESTs; Moderately similar to senne/threo | 2.81 | EB_cells, Lu_AD_H23, HT29_cells |
| 104938 | AA064627 | Hs.18341 | ESTs; Highly similar to CGI-72 protein [ | 2.81 | PC3_cells, HS578T_cells, OVCAR_cells |
| 128743 | AA237013 | Hs.2730 | heterogeneous nuclear ribonucleoprotein | 2.8 | OVCAR_cells, LNCaP_cells, Caco2 |
| 124314 | H94877 | Hs.215766 | GTP-binding protein | 2.8 | LNCaP_cells, DU145_cells, Caco2 |
| 134227 | D79986 | Hs.80338 | KIAA0164 gene product | 2.8 | LNCaP_cells, A549_cells, EB_cells |
| 122922 | AA476268 | | zw44h1.s1 Soares_total_fetus_Nb2HF8_9w H | 2.79 | Lu_SC_H345, OVCAR_cells, Lu_SC_H69 |
| | | | contains Alu repetitive element; contain | | |
| 128096 | H42968 | Hs.155606 | paired mesoderm homeo box 1 | 2.78 | Lu_AD_H23, Lu_SC_H69, Lu_LC_H460 |
| 129295 | AA424782 | Hs.110121 | SEC7 homolog | 2.78 | Lu_AD_H23, EB_cells, Lu_SC_H345 |
| 116155 | AA460957 | Hs.76053 | DEAD/H (Asp-Glu-Ala-Asp/His) box polypep | 2.78 | EB_cells, OVCAR_cells, 293T_cells |
| 105911 | AA401809 | Hs.189910 | ESTs | 2.77 | 293T_cells, HS578T_cells, DU145_cells |
| 119232 | T03475 | Hs.258624 | EST | 2.77 | EB_cells, Lu_AD_H23, Lu_AD_358 |
| 131168 | AA482007 | Hs.23788 | ESTs; Weakly similar to homology with is | 2.77 | EB_cells, Lu_LC_H460, MCF7 |
| 106048 | AA416697 | Hs.15330 | ESTs | 2.76 | OVCAR_cells, Lu_SC_H345, 293T_cells |
| 124352 | N21626 | Hs.102406 | ESTs | 2.76 | MCF7, MB-MDA-453, CALU6_cells |
| 129349 | D86974 | Hs.110613 | KIAA0220 protein | 2.76 | DU145_cells, HT29_cells, Lu_SC_H69 |
| 106120 | AA423808 | Hs.8765 | RNA helicase-related protein | 2.76 | OVCAR_cells, EB_cells, 293T_cells |
| 100643 | HG2755-H | | T-Plastin | 2.75 | 293T_cells, PC3_cells, HS578T_cells |
| 128500 | U60521 | Hs.100641 | caspase 9; apoptosis-releted cysteine pr | 2.75 | Lu_AD_358, Lu_SC_H69, Lu_SC_H345 |
| 126090 | R44789 | Hs.119486 | ESTs; Weakly similar to rostral cerebell | 2.75 | Lu_SC_H69, Lu_SC_H345, BT474_cells |
| 127064 | Z43709 | | HSC1JA091 normalized infant brain cDNA H | 2.75 | Caco2, A549_cells, HT29_cells |
| 132989 | AA480074 | Hs.394 | adrenomedullin | 2.75 | EB_cells, OVCAR_cells, DU145_cells |
| 108888 | AA135606 | Hs.189384 | ESTs; Weakly similar to !!!! ALU SUBFAMI | 2.75 | OVCAR_cells, LNCaP_cells, DU145_cells |
| 119579 | W42429 | Hs.150607 | ESTs | 2.74 | 293T_cells, DU145_cells, PC3_cells |
| 100387 | D83777 | Hs.75137 | KIAA0193 gene product | 2.74 | CALU6_cells, DU145_cells, Caco2 |
| 114744 | AA135407 | Hs.252351 | HERV-H LTR-associating 2 | 2.74 | PC3_cells, Lu_SQ_H520, RPWE_2 |
| 129092 | AA011243 | Hs.63525 | poly(rC)-binding protein 2 | 2.74 | EB_cells, MCF7, DU145_cells |
| 125360 | AA677978 | Hs.189741 | ESTs | 2.74 | Lu_AD_358, Lu_AD_358, PRSC_log |
| 107874 | AA025305 | Hs.25218 | ESTs; Weakly similar to reverse transcti | 2.74 | Lu_SC_H345, Lu_LC_H460, HT29_cells |
| 114086 | Z38266 | Hs.12770 | H sapiens PAC done DJ0777O23 from 7p14- | 2.74 | EB_cells, LNCaP_cells, BT474_cells |
| 116180 | AA463902 | Hs.94964 | ESTs | 2.73 | Lu_SC_H69, PRSC_con, Lu_AD_H23 |
| 126027 | M61982 | | ESTs | 2.73 | LNCaP_cells, DU145_cells, A549_cells |
| 116339 | AA496257 | Hs.72165 | ESTs; Weakly similar to R26984_1 [H.sapi | 2.73 | EB_cells, DU145_cells, OVCAR_cells |
| 105387 | AA236951 | Hs.108636 | chromosome 1 open reading frame 9 | 2.72 | PC3_cells, EB_cells, Caco2 |
| 111359 | N91273 | Hs.27179 | ESTs | 2.72 | EB_cells, LNCaP_cells, 293T_cells |
| 106680 | AA461458 | Hs.24789 | ESTs | 2.72 | PC3_cells, Lu_SC_H345, Caco2 |
| 118598 | N69136 | Hs.214343 | ESTs | 2.72 | MB-MDA-453, 293T_cells, BT474_cells |
| 107913 | AA027161 | Hs.59523 | ESTs; Highly similar to G1 TO S PHASE TR | 2.71 | EB_cells, MCF7, Lu_SC_H345 |
| 134315 | AA136269 | Hs.81648 | ESTs; Weakly similar to S164 [H.sapiens] | 2.71 | EB_cells, DU145_cells, HMEC |
| 135233 | AA127463 | Hs.9683 | protein-kinase; interferon-inducible dou | 2.71 | EB₁₃ cells, OVCAR_cells, Caco2 |
| 112932 | T15470 | Hs.189810 | ESTs | 2.7 | 293T_cells, Lu_AD_H23, PC3_cells |
| 119053 | R11501 | | yf28f1.s1 Soares fetal liver spleen 1NFL | 2.7 | Lu_SC_H345, Lu_SC_H69, DU145_cells |
| | | | contains Alu repetitive element; mRNA | | |
| 131206 | AA044078 | Hs.24210 | ESTs | 2.7 | Caco2, Lu_SC_H345, HS578T_cells |
| 126759 | AA063642 | | ESTs; Highly similar to (defline not ava | 2.7 | LNCaP_cells, Lu_SC_H345, LuSC_H69 |
| 131060 | AA160890 | Hs.22564 | myosin VI | 2.7 | LNCaP_cells, MCF7, HT29_cells |
| 132135 | N69101 | Hs.40730 | ESTs | 2.7 | EB_cells, 293T_cells, OVCAR_cells |
| 120835 | AA348446 | Hs.96906 | ESTs | 2.7 | Fibroblasts 2, CALU6_cells, RPWE_2 |
| 113815 | W45311 | Hs.14756 | ESTs | 2.7 | EB_cells, PC3_cells, DU145_cells |
| 133234 | T90092 | Hs.6853 | ESTs; Weakly similar to !!!! ALU SUBFAMI | 2.69 | Lu_SC_H345, OVCAR_cells, DU145_cells |
| 126819 | AA305536 | Hs.161489 | ESTs | 2.69 | EB_cells, DU145_cells, Caco2 |
| 125198 | W69474 | Hs.225550 | ESTs | 2.69 | Lu_SC_H345, Lu_AD_H23, Lu_AD_H23 |
| 108394 | AA075144 | | zm86f6.s1 Stratagene ovarian cancer (#93 | 2.69 | HMEC, HMEC (total RNA), Fibroblasts 2 |
| | | | gb:X1664 TRANSLATIONALLY CONTROLLED TUM | | |
| 134456 | X59405 | Hs.83532 | membrane cofactor protein (CD46; trophob | 2.69 | EB_cells, LNCaP_cells, DU145_cells |
| 111720 | R23739 | Hs.23585 | KIAA1078 protein | 2.88 | PC3_cells, HMEC (total RNA), OVCAR_cells |
| 114617 | AA084148 | Hs.110659 | ESTs | 2.68 | DU145_cells, LNCaP_cells, OVCAR_cells |
| 127787 | AA731764 | | ESTs; Weakly similar to !!!! ALU CLASS C | 2.68 | HT29_cells, Lu_SC_H345, MB231_cells |
| 101437 | M20681 | Hs.7594 | solute carrier family 2 (facilitated glu | 2.68 | Caco2, Lu_LC_H460, Fibroblasts 2 |
| 133761 | AA477223 | Hs.75922 | brain protein I3 | 2.68 | EB_cells, Lu_AD_H23, Lu_SC_H345 |
| 105869 | AA399574 | Hs.19086 | ESTs | 2.68 | PC3_cells, MCF7, MB231_cells |
| 125191 | W67257 | Hs.138871 | ESTs; Weakly similar to !!!! ALU CLASS B | 2.88 | OVCAR_cells, DU145_cells, LNCaP_cells |
| 116238 | AA479352 | Hs.47144 | DKFZP586N0819 protein | 2.67 | OVCAR_cells, DU145_cells, LNCaP_cells |
| 124770 | R40555 | Hs.120429 | ESTs | 2.67 | Lu_AD_H23, Lu_SC_H69, PRSC_con |
| 101764 | M80563 | Hs.81256 | S100 calcium-binding protein A4 (calcium | 2.67 | A549_cells, MB231_cells, OVCAR_cells |
| | | | murine placental homolog) | | |
| 130897 | AA063428 | Hs.21022 | adaptor-related protein complex 3; beta | 2.67 | EB_cells, Lu_AD_H23, HMEC |
| 133303 | H61048 | Hs.237352 | EST | 2.66 | Lu_SC_H345, Lu_SC_H69, PRSC_con |
| 124724 | R12405 | Hs.112423 | H sapiens mRNA; cDNA DKFZpS86I1420 (from | 2.66 | Lu_SC_H345, BT474_cells, OVCAR_cells |
| 123697 | AA609601 | Hs.221224 | ESTs | 2.66 | OVCAR_cells, 293T_cells, Lu_SC_H69 |
| 111548 | R09170 | Hs.258707 | ESTs | 2.66 | 293T_cells, CALU6_cells, A549_cells |
| 107005 | AA598679 | Hs.194215 | ESTs | 2.66 | Lu_SC_H345, OVCAR_cells, Lu_AD_H23 |
| 105569 | AA278399 | Hs.20596 | ESTs | 2.65 | MCF7, HT29_cells, BT474_cells |
| 132687 | AB002301 | Hs.54985 | KIAA0303 protein | 2.65 | HMEC (total RNA), HMEC, LNCaP_cells |
| 104105 | AA422123 | Hs.42457 | ESTs | 2.65 | Lu_SC_H345, Lu_SC_H69, DU145_cells |
| 121335 | AA404418 | Hs.144953 | ESTs | 2.65 | EB_cells, Fibroblasts 2, DU145_cells |
| 124853 | R61693 | Hs.172330 | ESTs; Weakly similar to Wiskott-Aldrich | 2.64 | Lu_SC_H69, 293T_cells, EB_cells |
| 124253 | H69742 | Hs.102201 | ESTs | 2.64 | DU145_cells, OVCAR_cells, Lu_SC_H345 |
| 123044 | AA481549 | Hs.165694 | ESTs | 2.64 | EB_cells, Lu_SC_H69, Lu_SC_H345 |
| 129535 | AA608852 | Hs.112603 | EST | 2.64 | EB_cells, Lu_AD_H23, Fibroblasts 2 |
| 131397 | AB002336 | Hs.26395 | erythrocyte membrane protein band 4.1-li | 2.64 | EB_cells, DU145_cells, Caco2 |
| 130175 | X75593 | Hs.151536 | RAB13; member RAS oncogene family | 2.64 | Fibroblasts 2, PRSC_con, HS578T_cells |
| 127507 | AI188445 | Hs.152618 | ESTs | 2.63 | EB_cells, Lu_AD_H23, Lu_LC_H460 |
| 105377 | AA236702 | Hs.24371 | ESTs | 2.63 | Caco2, EB₁₃ cells, CALU6_cells |
| 114671 | AA112679 | Hs.252291 | ESTs; Weakly similar to !!!! ALU SUBFAMI | 2.63 | EB_cells, DU145_cells, Caco2 |
| 133726 | W19983 | Hs.75761 | SFRS protein kinase 1 | 2.63 | EB_cells, Lu_AD_H23, Lu_SC_H69 |
| 132380 | H68018 | | yr76h05.r1 Soares fetal liver spleen 1NF | 2.62 | EB₁₃ cells, Lu_AD_H23, Lu_SC_H69 |
| | | | IMAGE:211257 5′, mRNA seq. | | |
| 127986 | AI370418 | Hs.192050 | ESTs; Weakly similar to !!!! ALU CLASS A | 2.62 | DU145_cells, OVCAR_cells, LNCaP_cells |
| 116208 | AA476333 | Hs.42532 | ESTs | 2.61 | DU145_cells, PRSC_con, Fibroblasts 2 |
| 130946 | AA069456 | Hs.21490 | KIAA0438 gene product | 2.6 | LNCaP_cells, DU145_cells, HS578T_cells |
| 106687 | AA463234 | Hs.119387 | KIAA0792 gene product | 2.59 | EB₁₃ cells, MB-MDA-453, Caco2 |
| 101551 | M31606 | Hs.196177 | phosphorylase kinase; gamma 2 (testis) | 2.59 | LNCaP_cells, EB_cells, MB-MDA-453 |
| 114479 | AA032084 | Hs.124841 | ESTs; Moderately similar to transformati | 2.59 | DU145_cells, Caco2, OVCAR_cells |
| 111863 | R37495 | Hs.23578 | ESTs | 2.59 | HT29_cells, MB231_cells, Lu_SQ_H520 |
| 129018 | AA029973 | Hs.107979 | small membrane protein 1 | 2.59 | A549_cells, EB_cells, HS578T_cells |
| 107058 | AA600357 | Hs.239409 | TIA1 cytotoxic granule-associated RNA-bi | 2.58 | DU145_cells, Lu_SC_H345, EB_cells |
| 126175 | AA056181 | Hs.17311 | DKFZP434N161 protein | 2.58 | Lu_SC_H345, DU145_cells, LNCaP_cells |
| 131979 | D52154 | Hs.172458 | iduronate 2-sulfatase (Hunter syndrome) | 2.58 | DU145_cells, PC3_cells, A549_cells |
| 126122 | H80181 | | ESTs | 2.58 | DU145_cells, OVCAR_cells, LNCaP_cells |
| 106961 | AA504110 | Hs.18063 | ESTs | 2.58 | HMEC, DU145_cells, DU145_cells |
| 114730 | AA133527 | Hs.126925 | ESTs; Weakly similar to The K1AA0138 gen | 2.58 | DU145_cells, LNCaP_cells, MCF7 |
| 117342 | N24020 | Hs.132913 | ESTs | 2.58 | HS578T_cells, DU145_cells, LNCaP_cells |
| 131622 | AA424813 | Hs.29692 | ESTs | 2.57 | PRSC_con, PRSC_log, HS578T_cells |
| 104904 | AA055560 | Hs.13179 | ESTs; Moderately similar to !!!! ALU SUB | 2.57 | Lu_SC_H345, Lu_SC_H69, BT474_cells |
| 117359 | N24848 | Hs.114062 | ESTs; Weakly similar to T15B7.2 [C.elega | 2.57 | HS578T_cells, PRSC_con, EB_cells |
| 123331 | AA497013 | Hs.188740 | ESTs; Weakly similar to !!!! ALU SUBFAMI | 2.57 | Lu_SC_H69, Caco2, PRSC_con |
| 125324 | R07785 | | yf15c06.r1 Soares fetal liver spleen 1NF | 2.57 | EB_cells, Lu_AD_H23, Fibroblasts 2 |
| | | | contains Alu repetitive element; contain | | |
| 129813 | T33462 | Hs.12600 | ESTs | 2.57 | Lu_SC_H345, 293T_cells, Lu_SC_H69 |
| 100265 | D38521 | Hs.75935 | KIAA0077 protein | 2.57 | EB_cells, LNCaP_cells, PC3_cells |
| 134890 | T40902 | Hs.90786 | ATP-binding cassette; sub-family C (CFTR | 2.57 | A549_cells, DU145_cells, EB_cells |
| 133582 | AA421874 | Hs.75087 | Fas-activated serine/threonine kinase | 2.56 | EB_cells, Lu_AD_H123, Lu_AD_358 |
| 135011 | H73161 | Hs.92991 | ESTs; Weakly similar to C13F10.4 [C.eleg | 2.56 | EB_cells, LNCaP_cells, MB-MDA-453 |
| 107226 | D58185 | Hs.21945 | ESTs | 2.56 | Lu_SC_H345, Lu_SC_H69, HMEC (total RNA) |
| 126042 | H62441 | Hs.157082 | H sapiens PAC clone DJ0988G15 from 7q33- | 2.56 | HMEC (total RNA), HMEC, RPWE_2 |
| 114472 | AA028924 | Hs.177407 | ESTs; Weakly similar to !!!! ALU SUBFAMI | 2.56 | Lu_SC_H345, Lu_SC_H69, DU145_cells |
| 126291 | N42090 | | yy05b07.r1 Soares melanocyte 2NbHM H sap | 2.56 | HMEC, HMEC (total RNA), PC3_cells |
| 113349 | T79021 | Hs.14438 | ESTs; Moderately similar to histamine N- | 2.56 | HT29_cells, PRSC_log, Lu_SC_H345 |
| 105789 | AA347485 | Hs.25477 | ESTs; Moderately similar to rig-1 protel | 2.56 | Lu_AD_H23, RPWE_2, Lu_SQ_H520 |
| 110918 | N46423 | Hs.24283 | ESTs | 2.56 | EB_cells, CALU6_cells, DU145_cells |
| 117170 | H98153 | Hs.42500 | ADP-ribosylation factor-like 5 | 2.56 | OVCAR_cells, EB_cells, LNCaP_cells |
| 105159 | AA173981 | Hs.30490 | CD2-associated protein | 2.55 | LNCaP_cells, EB_cells, DU145_cells |
| 105726 | AA292328 | Hs.9754 | activating transcription factor 5 | 2.55 | MCF7, EB_cells, MB-MDA-453 |
| 132079 | H67964 | Hs.38694 | ESTs | 2.55 | EB_cells, DU145_cells, HS578T_cells |
| 131813 | X51757 | Hs.3268 | heat shock 70 kD protein 6 (HSP70B′) | 2.55 | Lu_AD_H23, MB231_cells, Fibroblasts 2 |
| 133538 | L14837 | Hs.74614 | tight junction protein 1 (zona occludens | 2.54 | DU145_cells, Caco2, A549_cells |
| 124981 | T40849 | Hs.114034 | maternal G10 transcript | 2.54 | EB_cells, Caco2, LNCaP_cells |
| 122028 | AA431306 | Hs.98722 | ESTs | 2.54 | Fibroblasts 2, BT474_cells, HMEC (total RNA) |
| 122487 | AA448332 | Hs.80598 | transcription elongation factor A (SII); | 2.54 | Lu_SC_H345, MCF7, MB-MDA-453 |
| 119315 | T41152 | Hs.90485 | ESTs | 2.54 | Lu_SC_H345, MB-MDA-435s, PRSC_con |
| 107957 | AA031948 | Hs.57548 | ESTs | 2.54 | A549_cells, RPWE_2, DU145_cells |
| 122457 | AA447780 | Hs.96418 | ESTs | 2.54 | DU145_cells, EB_cells, A549_cells |
| 103572 | Z25749 | Hs.75538 | ribosomal protein S7 | 2.54 | EB_cells, CALU6_cells, DU145_cells |
| 124395 | N29963 | Hs.193977 | ESTs | 2.54 | HMEC (total RNA), HMEC, RPWE_2 |
| 116024 | AA451748 | Hs.83883 | Human DNA seq from clone 718J7 on chromo | 2.53 | LNCaP_cells, RPWE_2, MB-MDA-453 |
| | | | phosphoenolpyruvate carboxykinase 1; ES | | |
| 134361 | D43682 | Hs.82208 | acyl-Coenzyme A dehydrogenase; very long | 2.63 | LNCaP_cells, CALU6_cells, DU145_cells |
| 130420 | U60975 | | Human hybrid receptor gp25 precursor mRN | 2.53 | EB_cells, HMEC (total RNA), Caco2 |
| 100336 | D63478 | Hs.8127 | KIAA0144 gene product | 2.53 | BT474_cells, HT29_cells, Lu_AD_358 |
| 105519 | AA258063 | Hs.23438 | ESTs | 2.53 | EB_cells, Caco2, MB-MDA-435s |
| 124684 | R02401 | Hs.221078 | ESTs | 2.53 | Lu_SC_H345, OVCAR_cells, Lu_SC_H69 |
| 105852 | AA398933 | Hs.172613 | solute carrier family 12 (potassium/chlo | 2.52 | LNCaP_cells, DU145_cells, EB_cells |
| 105012 | AA116036 | Hs.9329 | chromosome 20 open reading frame 1 | 2.52 | CALU6_cells, Caco2, DU145_cells |
| 126534 | W39128 | Hs.247901 | Human DNA seq from clone 8B1 on chromoso | 2.52 | BT474_cells, LNCaP_cells, Lu_AD_H23 |
| | | | -CELL MEMBRANE GLYCOPROTEIN PC-1; the ge | | |
| 135334 | AA053134 | Hs.241558 | ariadne-2 (D. melanogaster) homolog (all | 2.52 | 293T_cells, CALU6_cells, DU145_cells |
| 128538 | R44214 | Hs.101189 | ESTs | 2.52 | EB_cells, Lu_AD_H23, Lu_SC_H345 |
| 109865 | H02566 | Hs.191268 | H sapiens mRNA; cDNA DKFZp434N174 (from | 2.52 | DU145_cells, LNCaP_cells, OVCAR_cells |
| 118579 | N68905 | | small inducible cytokine A5 (RANTES) | 2.51 | Lu_SC_H345, LNCaP_cells, Lu_SC_H69 |
| 117590 | N34904 | | ESTs; Moderately similarto !!!! ALU SUB | 2.51 | Lu_SC_H345, DU145_cells, Lu_SC_H69 |
| 104340 | F15201 | | ESTs | 2.51 | Lu_SC_H345, PRSC_con, PRSC_log |
| 122455 | AA447744 | Hs.99141 | EST | 2.51 | Caco2, Lu_SC_H69, 293T_cells |
| 109339 | AA211901 | Hs.86430 | ESTs | 2.51 | EB_cells, DU145_cells, CALU6_cells |
| 123258 | AA490929 | Hs.105274 | ESTs | 2.51 | EB_cells, Lu_AD_H23, Lu_SC_H69 |
| 118467 | N66763 | Hs.43080 | ESTs | 2.51 | CALU6_cells, HS578T_cells, OVCAR_cells |
| 106044 | AA416548 | Hs.149436 | kinesin family member 5B | 2.51 | EB_cells, Caco2, DU145_cells |
| 107480 | W58057 | Hs.74304 | periplakin | 2.5 | Caco2, OVCAR_ceUs, HMEC (total RNA) |
| 111760 | R26892 | Hs.221434 | ESTs | 2.5 | Lu_AD_H23, EB_cells, Lu_AD_358 |
| 132474 | N68018 | Hs.180930 | TBP-associated factor 172 | 2.5 | LNCaP_cells, EB_cells, DU145_cells |
| 103423 | X97249 | Hs.123122 | FSH primary response (LRPR1; rat) homolo | 2.5 | HS578T_cells, Lu_SC_H345, PC3_cells |
| 123488 | AA599708 | Hs.187764 | ESTs; Weakly similar to !!!! ALU SUBEAMI | 2.49 | OVCAR_cells, Lu_SC_H345, DU145_cells |
| 100475 | D90276 | Hs.12 | carcincembryonic antigen-related cell ad | 2.49 | MB-MDA-453, 293T_cells, CALU6_cells |
| 112003 | R42547 | Hs.172551 | ESTs | 2.49 | EB_cells, Lu_AD_H23, Lu_SC_H345 |
| 114315 | Z41027 | Hs.26297 | ESTs | 2.49 | Lu_SC_H69, OVCAR_cells, Lu_AD_H23 |
| 105291 | AA233311 | Hs.28752 | ESTs | 2.49 | EB_cells, CALU6_cells, DU145_cells |
| 135354 | AA188934 | Hs.99367 | ESTs | 2.49 | MB-MDA-453, Lu_SC_H69, 293T_cells |
| 107521 | X78262 | | H.sapiens mRNA for TRE5 | 2.49 | Lu_SC_H345, Lu_SC_H69, PRSC_con |
| 108373 | AA074393 | Hs.61950 | ESTs: Weakly similar to nuclear protein | 2.49 | MCF7, MB-MDA-453, Lu_SC_H345 |
| 108836 | AA132061 | Hs.222727 | ESTs; Weakly similar to ubiquitous TPR m | 2.48 | DU145_cells, Lu_SC_H345, Lu_SC_H345 |
| 110386 | H45516 | Hs.33268 | ESTs | 2.48 | PC3_cells, OVCAR_cells, Lu_SQ_Y520 |
| 129658 | M22348 | Hs.131255 | ubiquinol-cytochrome c reductase binding | 2.48 | LNCaP_cells, CALU6_cells, PC3_cells |
| 134283 | H12661 | Hs.8107 | H sapiens mRNA; cDNA DKFZpS86B0918 (from | 2.48 | HMEC (total RNA), HS578T_cells, HMEC |
| 101844 | M93425 | Hs.62 | protein tyrosine phosphatase; non-recept | 2.48 | DU145_cells, EB_cells, CALU6_cells |
| 133461 | M33318 | Hs.183584 | cytochrome P450; subfamily IIA (phenobar | 2.48 | EB_cells, Lu_AD_H23, Lu_AD_358 |
| 103545 | Z14000 | Hs.35384 | ring finger protein 1 | 2.47 | HT29_cells, Lu_SQ_H520, BT474_cells |
| 128440 | N76763 | | ESTs | 2.47 | EB_cells, Lu_AD_H23, Lu_AD_358 |
| 134992 | H05625 | Hs.92414 | ESTs | 2.47 | Lu_SC_H345, CALU6_cells, Lu_SC_H69 |
| 116295 | AA489016 | Hs.91216 | ESTs; Highly similar to partial CDS; hum | 2.47 | MB-MDA-453, 293T_cells, MB-MDA-435s |
| 107004 | AA598675 | Hs.239475 | ESTs | 2.47 | LNCaP_cells, Cace2, OVCAR_cells |
| 132137 | AA282312 | Hs.4076 | CTD (carboxy-terminal domain; RNA polyme | 2.46 | Lu_SC_H69, HMEC, EB_cells |
| 126390 | W28286 | Hs.100090 | tetraspan 3 | 2.46 | EB_cells, DU145_cells, LNCaP_cells |
| 113050 | T26366 | Hs.22711 | EST; Weakly similar to 60S RIBOSOMAL PRO | 2.46 | Lu_LC_H460, EB_cells, Lu_AD_358 |
| 101667 | M60858 | Hs.79110 | nucleolin | 2.46 | PC3_cells, 293T_cells, A549_cells |
| 108569 | AA085398 | | zn7e3.s1 Stratagene hNT neuron (#937233) | 2.45 | HT29_cells, BT474_cells, Lu_SQ_H520 |
| | | | IMAGE:546748 3′, mRNA seq | | |
| 117186 | H98988 | Hs.42612 | ESTs | 2.45 | EB_cells, Lu_AD_H23, Lu_AD_358 |
| 129091 | AA044622 | Hs.183755 | Human Chromosome 16 BAG clone CIT987SK-A | 2.45 | EB_cells, Lu_AD_H23, Lu_AD_H23 |
| 128468 | T23625 | Hs.258674 | EST | 2.45 | Lu_AD_H23, EB_cells, Lu_SC_H69 |
| 117498 | N31726 | Hs.44268 | ESTs; Highly similar to myelin gene expr | 2.45 | Lu_SC_H69, DU145_cells, OVCAR_cells |
| 105407 | AA243478 | Hs.5206 | ESTs | 2.45 | EB_cells, 293T_cells, PC3_cells |
| 128941 | R55763 | Hs.107287 | ESTs | 2.44 | EB_cells, LNCaP_cehls, A549_cells |
| 116486 | C14128 | Hs.251980 | EST | 2.44 | MB-MDA-435s, HS578T_cells, 293T_cells |
| 134869 | T35288 | Hs.90421 | ESTs; Moderately similar to !!!! ALU SUB | 2.44 | EB_cells, Lu_AD_H23, Lu_AD_358 |
| 130664 | R09049 | Hs.17625 | ESTs | 2.44 | PC3_cells, EB_cells, A549_cells |
| 107985 | AA035638 | Hs.71968 | H sapiens mRNA; cDNA DKFZpS54F053 (from | 2.44 | PRSC_con, PRSC_log, Caco2 |
| 110300 | H37820 | Hs.124147 | ESTs | 2.44 | MB-MDA-453, Caco2, OVCAR_cells |
| 113471 | T87174 | Hs.16341 | ESTs; Moderately similar to !!!! ALU SUB | 2.44 | Caco2, OVCAR_cells, LNCaP_cells |
| 131474 | U28749 | Hs.2726 | high-mobility group (nonhistone chromoso | 2.44 | CALU6_cells, OVCAR_cells, 293T_cells |
| 120791 | AA342802 | Hs.194031 | ESTs | 2.44 | Lu_AD_H23, Lu_SQ_H520, PRSC_con |
| 133733 | AA416973 | Hs.75798 | Human DNA seq from clone 1183I21 on chro | 2.43 | EB_cells, Caco2, DU145_cells |
| | | | to predicted fly and worm proteins. Con | | |
| 119977 | W88579 | Hs.124744 | ESTs | 2.43 | HT_cells, HMEC (total RNA), HMEC |
| 134921 | W60186 | Hs.169487 | Kreisler (mouse) maf-related leucine zip | 2.43 | LNCaP_cells, HS578T_cells, MB-MDA-453 |
| 132295 | H66351 | Hs.181042 | Dmx-hike 1 | 2.43 | Lu_SC_H69, BT474_cells, Lu_SQ_H520 |
| 133395 | AA491296 | Hs.72805 | ESTs | 2.43 | EB_cells, LNCaP_cells, OVCAR_cells |
| 106728 | AA465355 | Hs.153768 | U3 snoRNP-associated 55-kDa protein | 2.43 | EB_cells, Lu_AD_H23, PC3_cells |
| 116370 | AA521256 | Hs.236204 | ESTs; Moderately similar to NUCLEAR PORE | 2.43 | EB_cells, A549_cells, 293T_cells |
| 113936 | W81552 | Hs.83623 | nuclear receptor subfamily 1; group I; m | 2.43 | 293T_cells, OVCAR_cells, Fibroblasts 2 |
| 128862 | R61297 | Hs.106673 | eukaryotic translation initiation factor | 2.43 | EB_cells, DU145_cells, DU145_cells |
| 111614 | R12581 | Hs.191146 | ESTs | 2.43 | HMEC (total RNA), Fibroblasts 2, MB-MDA-435s |
| 111993 | R42241 | Hs.106359 | ESTs | 2.43 | A549_cells, DU145_cells, CALU6_cells |
| 131554 | AA100026 | Hs.28669 | ESTs; Weakly similar to PROTEIN-TYROSINE | 2.43 | EB_cells, LNCaP_cells, Caco2 |
| 130983 | N71215 | Hs.21862 | NCK-associated protein 1 | 2.42 | EB_cells, Caco2, A549_cells |
| 131654 | AA497050 | Hs.30204 | ESTs | 2.42 | MCF7, MB-MDA-435s, Lu_SC_H345 |
| 105014 | AA121123 | Hs.191374 | ESTs | 2.42 | EB_cells, Lu_AD_H23, Lu_LC_H460 |
| 106300 | AA435840 | Hs.19114 | high-mobility group (nonhistone chromoso | 2.42 | EB_cells, LuSC_H345, A549_cells |
| 102386 | U40998 | Hs.81728 | unc119 (C.elegans) homolog | 2.42 | OVCAR_cells, EB_cells, DU145_cells |
| 112517 | R68589 | Hs.23721 | ESTs | 2.42 | Caco2, MCF7, DU145_cells |
| 125375 | H72971 | | KIAA0277 gene product | 2.42 | Lu_SC_H345, OVCAR_cells, Lu_SC_H69 |
| 123808 | AA620552 | Hs.25682 | ESTs; Weakly similar to PHOSPHATIDYLETHA | 2.42 | EB_cells, Lu_AD_H23, Lu_SC_H69 |
| 114950 | AA243503 | Hs.11801 | adenosine A2b receptor pseudogene | 2.42 | MB-MDA-453, HT29_cells, Lu_LC_H460 |
| 129906 | H39216 | Hs.239970 | ESTs; Weakly similar to ZNF91L [H.sapien | 2.41 | Lu_SC_H345, Fibroblasts 2, DU145_cells |
| 103408 | X95876 | Hs.198252 | G protein-coupled receptor 9 | 2.41 | RPWE_2, PRSC_log, Lu_SC_H345 |
| 129703 | AA401348 | Hs.179999 | ESTs | 2.41 | EB_cells, 293T_cells, DU145_cells |
| 105693 | AA287104 | Hs.181368 | U5 snRNP-speciflc protein (220 kD); orth | 2.41 | 293T_cells, CALU6_cells, A549_cells |
| 106532 | AA453628 | Hs.37443 | ESTs | 2.41 | ES_cells, OVCAR_cells, Caco2 |
| 132132 | AA010933 | Hs.4055 | core promoter element binding protein | 2.41 | HMEC, HMEC (total RNA), EB_cells |
| 111409 | R00311 | Hs.18798 | EST; Weakly similar to !!!! ALU SUBFAMIL | 2.41 | Lu_SC_H345, Lu_SC_H69, PRSC_con |
| 133813 | M26657 | Hs.250711 | dipeptidyl carboxypeptidase 1 (angiotens | 2.41 | HT29_cells, BT474_cells, MB231_cells |
| 127240 | AA888387 | Hs.243845 | ESTs; Moderately similar to !!!! ALU SUB | 2.41 | Lu_SC_H345, DU145_cells, LNCaP_cells |
| 104975 | AA086071 | Hs.50758 | chromosome-associated polypeptide C | 2.41 | OVCAR_cells, DU145_cells, PC3_cells |
| 118078 | N54321 | Hs.47790 | EST | 2.41 | EB_cells, Fibroblasts 2, HMEC (total RNA) |
| 115840 | AA429253 | Hs.58103 | A kinase (PRKA) anchor protein 9 | 2.41 | OVCAR_cells, EB_cells, PC3_cells |
| 101186 | L20298 | Hs.179881 | core-binding factor; beta subunit | 2.4 | EB_cells, DU145_cells, CALU6_cells |
| 113098 | T40936 | Hs.8349 | ESTs | 2.4 | Caco2, HT_cells, EB_cells |
| 115185 | AA259140 | Hs.60238 | ESTs | 2.4 | Lu_SC_H69, EB_cells, Caco2 |
| 113778 | W15263 | Hs.5422 | ESTs | 2.4 | Caco2, MB-MDA-435s, LNCaP_cells |
| 128261 | AI061213 | Hs.13179 | ESTs; Moderately similar to !!!! ALU SUB | 2.4 | DU145_cells, LNCaP_cells, OVCAR_cells |
| 132210 | AA235013 | Hs.42322 | A kinase (PRKA) anchor protein 2 | 2.4 | Caco2, DU145_cells, PRSC_log |
| 112561 | R72427 | Hs.129873 | ESTs; Weakly similar to CYTOCHROME P450 | 2.4 | Lu_SQ_H520, Lu_AD_H23, EB_cells |
| 127598 | AA610677 | Hs.168851 | ESTs | 2.4 | LNCaP_cells, DU145_cells, OVCAR_cells |
| 106664 | AA460969 | Hs.7510 | mitogen-activated protein kinase kinase | 2.4 | OVCAR_cells, 293T_cells, A549_cells |
| 131367 | AA456687 | Hs.26057 | ESTs | 2.4 | EB_cells, MB-MDA-453, 293T_cells |
| 103163 | X67683 | | H.sapiens mRNA for keratin 4 | 2.39 | EB_cells, LuAD_H23, Lu_AD_358 |
| 109639 | F04444 | Hs.6217 | ESTs; Weakly similar to !!!! ALU SUSFAMI | 2.39 | EB_cells, Lu_SC_H345, Lu_SC_H69 |
| 112007 | R42671 | Hs.140853 | EST; Weakly similar to !!!! ALU SUBFAMIL | 2.39 | MB-MDA-435s, Lu_SC_H345, Lu_AD_H23 |
| 100023 | | | AFFX control: BioC-3 | 2.39 | Caco2, Lu_AD_358, LNCaP_cells |
| 119923 | W86214 | Hs.184642 | ESTs | 2.39 | EB_cells, HS578T_cells, DU145_cells |
| 127705 | AJ003307 | | AJ003307 Selected dir 21 cDNA library H | 2.39 | Lu_AD_H23, Lu_SC_H345, Lu_LC_H460 |
| 130362 | AA182658 | Hs.179817 | DKFZP586F0222 protein | 2.39 | EB_cells, DU145_cells, PC3_cells |
| 100168 | D14874 | Hs.394 | adrenomedullin | 2.39 | Fibroblasts 2, Caco2, HS578T_cells |
| 134261 | AA227678 | Hs.8084 | Human DNA seq from clone 465N24 on chr 1 | 2.39 | PRSC_con, MB-MDA-453, LNCaP_cells |
| | | | Contains two novel genes; ESTs; GSSs an | | |
| 103392 | X94563 | | H.sapiens dbi/acbp gene exon 1 & 2 | 2.38 | ES_cells, Lu_AD_H23, Lu_SC_H69 |
| 129888 | U81001 | Hs.131891 | Human SNRPN mRNA; 3′ UTR; partial seq | 2.38 | LNCaP_cells, Lu_SC_H69, Lu_LC_H460 |
| 130119 | T12649 | Hs.251653 | tubulin; beta; 2 | 2.38 | Lu_AD_H23, Lu_LC_H460, Lu_LC_H460 |
| 118136 | N57710 | Hs.233952 | proteasome (prosome; macropain) subunit | 2.38 | 293T_cells, OVCAR_cells, HS578T_cells |
| 131163 | H80107 | Hs.23754 | ESTs | 2.38 | Lu_AD_H23, Lu_SC_H69, Lu_SC_H345 |
| 115964 | AA448622 | Hs.74313 | ESTs | 2.38 | EB_cells, LNCaP_cells, DU145_cells |
| 135026 | H59730 | Hs.93231 | ESTs | 2.37 | EB_cells, 293T_cells, Lu_SC_H69 |
| 133300 | D51401 | Hs.70333 | ESTs | 2.37 | OVCAR_cells, Caco2, CALU6_cells |
| 129948 | H69281 | Hs.13643 | ESTs | 2.37 | EB_cells, Lu_AD_H23, Lu_SC_H345 |
| 112505 | R67923 | Hs.23368 | ESTs | 2.37 | DU145_cells, OVCAR_cells, 293T_cells |
| 130715 | T98227 | Hs.171952 | occludin | 2.37 | Caco2, LNCaP_cells, DU145_cells |
| 120301 | AA192163 | Hs.104085 | EST | 2.37 | Lu_AD_H23, EB_cells, PRSC_con |
| 128062 | AA379500 | Hs.193155 | ESTs | 2.37 | EB_cells, LNCaP_cells, DU145_cells |
| 127154 | AA789101 | Hs.198860 | ESTs; Weakly similar to !!!! ALU SUBFAMI | 2.37 | HS578T_cells, MCF7, Lu_SC_H69 |
| 102814 | U90716 | Hs.79187 | coxsackie virus and adenovirus receptor | 2.37 | OVCAR_cells, DU145_cells, Lu_SC_H345 |
| 120239 | Z41691 | Hs.65919 | ESTs | 2.37 | EB₁₃ cells, DU145_cells, LNCaP_cells |
| 106829 | AA481883 | Hs.31236 | ESTs; Weakly similar to Unknown [H.sapie | 2.37 | EB_cells, DU145_cells, OVCAR_cells |
| 132681 | AA435762 | Hs.54894 | ESTs; Highly similar to unknown [H.sapie | 2.37 | EB_cells, LNCaP_Cells, PRSC_con |
| 108845 | AA132946 | Hs.68864 | ESTs | 2.36 | Lu_AD_H23, Lu_AD_358, Lu_SQ_H520 |
| 133226 | T85327 | Hs.169552 | ESTs | 2.36 | Caco2, MB-MDA-453, MCF7 |
| 106789 | AA478726 | Hs.26373 | ESTs; Moderately similar to !!!! ALU SUB | 2.36 | MS-MDA-453, Caco2, OVCAR_cells |
| 119236 | T10166 | Hs.237297 | ESTs | 2.36 | EB_cells, 293T_cells, LNCaP_cells |
| 106619 | AA459255 | Hs.23956 | ESTs | 2.36 | LNCaP_cells, A549_cells, Caco2 |
| 109178 | AA181600 | Hs.62741 | ESTs | 2.36 | Lu_SC_H345, LNCaP_cells, EB_cells |
| 112724 | R91753 | Hs.17757 | ESTs | 2.36 | Caco2, EB_cells, DU145_cells |
| 112655 | R85069 | Hs.141139 | ESTs | 2.36 | Fibroblasts 2, Lu_AD_H23, Lu_LC_H460 |
| 132820 | AA454988 | Hs.57621 | ESTs | 2.36 | EB_cells, OVCAR_cells, HS578T_cells |
| 106155 | AA425309 | Hs.33287 | nuclear factor I/B | 2.36 | OVCAR_cells, Lu_SC_H345, MB-MDS-453 |
| 114632 | AA084742 | Hs.194380 | ESTs; Weakly similar to !!!! ALU SUSFAMI | 2.35 | Lu_SC_H345, Lu_LC_H460, Lu_AD_H23 |
| 134776 | J05582 | Hs.89603 | mucin 1; transmembrane | 2.35 | DU145_cells, Lu_AD_H23, Lu_AD_358 |
| 101192 | L20859 | Hs.78452 | solute carrier family 20 (phosphate tran | 2.35 | PC3_cells, CALU6_cells, MS-MDA-435s |
| 130349 | W16686 | Hs.171825 | basic helix-loop-helix domain containing | 2.35 | A649_cells, DU145_cells, HT29_cells |
| 106389 | AA448949 | Hs.6236 | ESTs | 2.36 | LNCaP_cells, PC3_cells, DU145_cells |
| 109637 | F04426 | Hs.23131 | kinesin family member C3 | 2.35 | MB-MDA-435s, A549_cells, Lu_LC_H460 |
| 101483 | M24486 | Hs.76768 | procollagen-proline; 2-oxoglutarate 4-di | 2.35 | PC3_cells, HS578T_cells, EB_cells |
| 131751 | H18335 | Hs.31562 | ESTs | 2.35 | DU145_cells, MB231_cells, HMEC |
| 131050 | X13967 | Hs.2250 | leukemia inhibitory factor (cholinergic | 2.35 | Lu_AD_H23, PC3_cells, PRSC_log |
| 130097 | N21159 | Hs.14845 | forkhead box O3A | 2.34 | EB_cells, LNCaP_cells, LNCaP_cells |
| 134533 | AA013468 | Hs.241493 | natural killer-tumor recognition seq | 2.34 | EB_cells, HT29_cells, HMEC |
| 134839 | D63479 | Hs.115907 | diacylglycerol kinase; delta (130 kD) | 2.34 | Lu_LC_H460, Caco2, DU145_cells |
| 115690 | AA410894 | Hs.44159 | ESTs | 2.34 | PC3_cells, EB_cells, OVCAR_cells |
| 129079 | N91011 | Hs.108502 | ESTs | 2.34 | Lu_AD_H23, Lu_SC_H69, Lu_AD_358 |
| 123517 | AA608525 | Hs.243059 | EST | 2.34 | Lu_SC_H345, PC3_cells, MS-MDA-435s |
| 126239 | AA527215 | Hs.75879 | ribosomal protein L19 | 2.34 | BT474_cells, Lu_LC_H460, Lu_AD_H23 |
| 124440 | N46435 | | ESTs | 2.34 | Lu_SC_H69, HT29_cells, MS-MDA-435s |
| 111468 | R05809 | Hs.205481 | ESTs | 2.34 | Lu_AD_H23, PRSC_log, Lu_SQ_H520 |
| 129560 | H18428 | Hs.113613 | ESTs; Moderately similar to !!!! ALU SUB | 2.34 | Lu_SC_H69, Lu_SC_H345, LNCaP_cells |
| 104857 | AA043219 | Hs.19058 | ESTs | 2.34 | Lu_AD_H23, Lu_SC_H345, Lu_SC_H345 |
| 109647 | F04587 | Hs.28241 | ESTs | 2.34 | HS578T_cells, A549_cells, CALU6_cells |
| 117160 | H97817 | Hs.183302 | ESTs | 2.34 | EB_cells, Fibroblasts 2, Lu_SC_H69 |
| 112352 | R58974 | Hs.167343 | ESTs | 2.34 | EB_cells, Lu_SC_H345, HT29_cells |
| 113653 | T95745 | Hs.187433 | ESTs | 2.34 | MB-MDA-435s, MB-MDA-453, Lu_SC_H345 |
| 131606 | W56804 | Hs.29385 | AFG3 (ATPase family gene 3; yeast)-like | 2.34 | OVCAR_cells, Fibroblasts 2, MB-MDA-435s |
| 101525 | M29536 | Hs.12163 | eukaryotic translation initiation factor | 2.34 | EB_cells, Caco2, DU145_cells |
| 125921 | AA775029 | Hs.122591 | ESTs | 2.33 | 293T_cells, PRSC_log, Lu_SC_H345 |
| 125775 | AA213555 | Hs.29205 | alpha integrin binding protein 63 | 2.33 | EB_cells, DU145_cells, LNCaP_cells |
| 108743 | AA126917 | Hs.71074 | ESTs | 2.33 | Lu_AD_H23, Lu_AD_358, Lu_LC_H460 |
| 133735 | AC002045 | Hs.251928 | nuclear pore complex interacting protein | 2.33 | LNCaP_cells, Lu_SC_H69, DU145_cells |
| 120403 | AA234916 | Hs.243851 | ESTs | 2.33 | MB231_cells, Lu_SC_H345, Lu_SC_H69 |
| 134998 | R02207 | Hs.92679 | ESTs; Weakly similar to microtubule-base | 2.33 | LNCaP_cells, BT474_cells, MCF7 |
| 108456 | AA079326 | Hs.143654 | ESTs | 2.33 | HT29_cells, Lu_AD_H23, RPWE_2 |
| 130552 | M86667 | Hs.179662 | nucleosome assembly protein 1-like 1 | 2.33 | EB_cells, A549_cells, DU145_cells |
| 111114 | N63391 | Hs.9238 | ESTs | 2.33 | Caco2, EB_cells, MB-MDA-453 |
| 127767 | AI269498 | Hs.125543 | ESTs; Moderately similar to TADA1 protei | 2.33 | CALU6_cells, 293T_cells, PC3_cells |
| 106546 | AA454725 | Hs.21056 | H sapiens mRNA from chromosome 5q21-22; | 2.33 | OVCAR_cells, Caco2, LNCaP_cells |
| 122379 | AA446110 | Hs.250989 | EST | 2.33 | BT474_cells, Fibroblasts 2, MB-MDA-435s |
| 133650 | D84294 | Hs.118174 | tetratricopeptide repeat domain 3 | 2.33 | Lu_SC_H345, EB_cells, EB_cells |
| 106434 | AA449099 | Hs.8151 | ESTs; Weakly similar to atopy related au | 2.33 | EB_cells, LNCaP_cells, Caco2 |
| 105297 | AA233451 | Hs.183858 | transcriptional intermediary factor 1 | 2.33 | EB_cells, LNCaP_cells, Caco2 |
| 115976 | AA447442 | Hs.86327 | ESTs | 2.33 | EB_cells, 293T_cells, Lu_SC_H69 |
| 105788 | AA351031 | Hs.23965 | solute carrier family 22 (organic anion | 2.33 | EB_cells, Lu_AD_H23, Lu_SC_H345 |
| 113774 | W04550 | Hs.9927 | H sapiens mRNA; cDNA DKFZp564D156 (from | 2.32 | OVCAR_cells, EB_cells, Lu_SC_H69 |
| 110617 | H68772 | Hs.35820 | ESTs; Weakly similar to b3418.1 [H.sapie | 2.32 | Lu_SC_H345, Lu_AD_H23, PRSC_con |
| 102234 | U26312 | Hs.8123 | chromobox homolog 3 (Drosophila HP1 gamm | 2.32 | CALU6_cells, LNCaP_cells, A549_cells |
| 114777 | AA151699 | Hs.184519 | ESTs; Weakly similar to !!!! ALU SUBFAMI | 2.32 | HT29_cells, Fibroblasts 2, Lc_SC_H345 |
| 125518 | R20148 | Hs.193851 | ESTs | 2.32 | HT29_cells, HMEC (total RNA), MB231_cells |
| 130814 | AA256695 | Hs.19813 | ESTs | 2.32 | MB-MDA-435s, Lu_SC_H69, PRSC_log |
| 123473 | AA599143 | | ESTs; Moderately similar to !!!! ALU SUB | 2.32 | LNCaP_cells, DU145_cells, Lu_H345 |
| 134310 | AA313414 | Hs.8148 | H sapiens clone 24856 mRNA seq; complete | 2.32 | PC3_cells, LNCaP_cells, OVCAR_cells |
| 119192 | R85375 | Hs.237262 | EST | 2.32 | Lu_SC_H69, PRSC_log, PRSC_con |
| 114391 | AA004876 | Hs.133100 | ESTs | 2.32 | PC3_cells, 293T_cells, 293T_cells |
| 119133 | R49144 | Hs.119756 | ESTs | 2.32 | PRSC_log, 293T_cells, 293T_cells |
| 109710 | F09792 | Hs.12929 | ESTs | 2.32 | Lu_AD_H23, Lu_SC_H69, Lu_SC_H345 |
| 116726 | F13681 | Hs.42309 | ESTs | 2.32 | MCF7, BT474_cells, MB-MDA-453 |
| 133206 | R32993 | Hs.6762 | ESTs; Weakly similar to similar to leucy | 2.31 | DU145_cells, 293T_cells, EB_cells |
| 135163 | AA125988 | Hs.199955 | ESTs | 2.31 | Lu_SC_H345, LNCaP_cells, DU145_cells |
| 111219 | N68836 | Hs.19247 | ESTs | 2.31 | OVCAR_cells, LNCaP_cells, 293T_cells |
| 110283 | H29565 | Hs.12271 | ESTs | 2.31 | BT474_cells, MB231_cells, MB231_cells, MB-MDA-453 |
| 103772 | AA092473 | Hs.8123 | chromobox homolog 3 (Drosophila HP1 gamm | 2.31 | CALU6_cells, MCF7, DU145_cells |
| 122766 | AA459386 | Hs.194058 | ESTs; Weakly similar to atypical PKC spe | 2.31 | HT_cells, BT474_cells, HMEC |
| 120886 | AA365566 | Hs.132736 | ESTs; Weakly similarto allograft inflam | 2.31 | DU145_cells, A549_cells, Lu_LC_H460 |
| 123512 | AA600248 | Hs.142245 | HERV-H LTR-associating 3 | 2.31 | PC3_cells, 293T_cells, DU145_cells |
| 106644 | AA460239 | Hs.12680 | ESTs | 2.31 | HS578T_cells, MB231_cells, Lu_SQ_H520 |
| 127359 | H72971 | | KIAA0277 gene product | 2.31 | Lu_SC_H345, DU145_cells, OVCAR_cells |
| 105919 | AA402494 | Hs.3990 | ESTs | 2.31 | HS578T_cells, DU145_cells, LNCaP_cells |
| 125241 | W86291 | Hs.121593 | ESTs | 2.3 | HMEC, HMEC (total RNA), EB_cells |
| 104624 | AA001936 | Hs.184721 | ESTs | 2.3 | DU145_cells, PC3_cells, PRSC_log |
| 128765 | AA101767 | Hs.10494 | ESTs | 2.3 | EB_cells, HMEC (total RNA), Lu_LC_H460 |
| 108360 | AA071539 | | zm74b6.s1 Stratagene neuroepithelium (#9 | 2.3 | HT29_cells, RPWE_2, Lu_AD_H23 |
| | | | HYDROXYSTEROID DEHYDROGENASEIDELTA-5-DEL | | |
| 115682 | AA410300 | Hs.44618 | ESTs | 2.3 | HT29_cells, Lu_SQ_H520, Lu_AD_H23 |
| 134528 | M23161 | Hs.84775 | Human transposon-like element mRNA | 2.3 | EB_cells, CALU6_cells, A549_cells |
| 111091 | N59858 | Hs.33032 | H sapiens mRNA; cDNA DKFZp434N185 (from | 2.3 | LNCaP_cells, DU145_cells, PRSC_log |
| 134044 | AA262475 | Hs.78746 | phosphodiesterase 8A | 2.29 | DU145_cells, A549_cells, MCF7 |
| 118229 | N62339 | Hs.180532 | heat shock 90 kD protein 1; alpha | 2.29 | MCF7, DU145_cells, EB_cells |
| 110188 | H20522 | Hs.20969 | ESTs | 2.29 | Fibroblasts 2, MB-MDA-435s, Lu_LC_H460 |
| 125073 | T87185 | Hs.193638 | ESTs; Weakly similar to !!!! ALU CLASS C | 2.29 | EB_cells, Lu_SC_H345, Lu_SC_H69 |
| 111495 | R07210 | Hs.19913 | ESTs | 2.29 | CALU6_cells, EB_cells, MCF7 |
| 124024 | F03077 | Hs.106672 | ESTs | 2.29 | HS578T_cells, RPWE_2, Lu_AD_358 |
| 128230 | AA984074 | Hs.176757 | ESTs | 2.29 | LNCaP_cells, DU145_cells, OVCAR_cells |
| 125471 | AA477571 | Hs.152601 | UDP-glucose ceramide glucosyltransferase | 2.29 | DU145_cells, PRSC_con, PRSC_log |
| 120734 | AA299949 | | EST12545 Uterus tumor I H sapiens cDNA 3 | 2.28 | Lu_AD_H23, Lu_SC_H345, Lu_SC_H69 |
| 134349 | AA406373 | Hs.8208 | ESTs | 2.28 | DU145_cells, PC3_cells, LNCaP_cells |
| 123412 | AA521443 | Hs.187763 | ESTs | 2.28 | BT474_cells, BT474_cells, Lu_SC_H169 |
| 116297 | AA489042 | Hs.59498 | ESTs | 2.28 | EB_cells, 293T_cells, MB-MDA-453 |
| 104476 | N33807 | Hs.223014 | protease; serine; 15 | 2.28 | LNCaP_cells, MCF7, PC3_cells |
| 101004 | J04101 | Hs.248109 | v-ets avian erythroblastosis virus E26 o | 2.28 | HT29_cells, MB-MDA-435s, HMEC (total RNA) |
| 109991 | H09813 | Hs.12896 | KIAA1034 protein | 2.28 | EB_cells, CALU6_cells, 293T_cells |
| 118934 | N92571 | Hs.54808 | ESTs | 2.28 | HS578T_cells, 293T_cells, A549_cells |
| 125096 | T94328 | Hs.194533 | ESTs | 2.28 | Lu_SC_H345, Lu_SC_H69, 293T_cells |
| 117514 | N32226 | Hs.124058 | ESTs | 2.28 | CALU6_cells, HMEC, Lu_AD_H23 |
| 132792 | AA401903 | Hs.242985 | hemoglobin; gamma G | 2.28 | OVCAR_cells, Lu_SC_H69, MCF7 |
| 129009 | AA131421 | Hs.107884 | ESTs | 2.28 | Hs578T_cells, CALU6_cells, Caco2 |
| 111658 | R16981 | Hs.15276 | ESTs | 2.28 | MB-MDA-435s, 293T_cells, A549_cells |
| 112322 | R55757 | Hs.26457 | EST | 2.28 | Lu_SC_H345, Lu_SC_H69, Lu_AD_358 |
| 133477 | W69310 | Hs.740 | PTK2 protein tyrosine kinase 2 | 2.28 | EB_cells, PC3_cells, DU145_cells |
| 132149 | T10822 | Hs.4095 | ESTs | 2.28 | LNCaP_cells, EB_cells, PC3_cells |
| 115119 | AA256524 | Hs.46847 | Human DNA seq from done 30M3 on chromos | 2.27 | A549_cells, EB_cells, LNCaP_cells |
| | | | yeast and archaea bacterial genes; and | | |
| 102130 | U15009 | Hs.1575 | small nuclear ribonucleoprotein D3 polyp | 2.27 | LNCaP_cells, Caco2, EB_cells |
| 114343 | Z41424 | Hs.21259 | ESTs | 2.27 | HT29_cells, OVCAR_cells, Fibroblasts 2 |
| 106746 | AA476436 | Hs.7991 | ESTs | 2.27 | Lu_AD_358, RPWE_2, Lu_AD_H23 |
| 119359 | T71021 | Hs.93334 | ESTs; Highly similar to WS basic-helix-I | 2.27 | Lu_SC_H69, 293T_cells, DU145_cells |
| 106301 | AA435867 | Hs.168212 | kinesin family member 3B | 2.27 | OVCAR_cells, LNCaP_cells, EB_cells |
| 130280 | L13738 | Hs.153937 | activated p21cdc42Hs kinase | 2.27 | MB-MDA-453, DU145_cells, DU145_cells |
| 119724 | W69468 | Hs.47622 | ESTs | 2.27 | PC3_cells, HT29_cells, A549_cells |
| 108960 | AA150199 | Hs.49378 | DKFZP586D0919 protein | 2.27 | EB_cells, HS578T_cells, Lu_AD_358 |
| 103489 | Y08614 | Hs.79090 | exportin 1 (CRM1; yeast; homolog) | 2.26 | EB_cells, CALU6_cells, DU145_cells |
| 107711 | AA015736 | Hs.220687 | ESTs | 2.26 | EB_cells, Lu_AD_H23, Lu_AD_358 |
| 131950 | W84704 | Hs.35380 | ESTs | 2.26 | HS578T_cells, OVCAR_cells, MB-MDA-435s |
| 107093 | AA609600 | Hs.10018 | ESTs | 2.26 | LNCaP_cells, OVCAR_cells, DU145_cells |
| 113649 | T95641 | Hs.16400 | ESTs; Weakly similar to Hrs [H.sapiens] | 2.26 | Lu_AD_H23, Lu_SC_H69, PRSC_log |
| 105255 | AA227498 | Hs.3623 | ESTs | 2.26 | HS578T_cells, 293T_cells, Lu_SC_H345 |
| 130094 | H43286 | Hs.167017 | gamma-aminobutyric acid (GABA) B recepto | 2.26 | Fibroblasts 2, MB231_cells, 293T_cells |
| 111874 | R37959 | Hs.13358 | ESTs | 2.26 | CALU6_cells, Lu_SQ_H520, 293T_cells |
| 107890 | AA026030 | Hs.61311 | ESTs; Weakly similar to CALPAIN 2; LARGE | 2.26 | HT29_cells, MB-MDA-453, PC3_cells |
| 124628 | N74702 | Hs.102834 | ESTs | 2.26 | 293T_cells, CALU6_cells, CALU6_cells |
| 119707 | W67569 | Hs.44143 | ESTs; Weakly similar to SNF2alpha protei | 2.26 | 293T_cells, OVCAR_cells, Lu_SC_H345 |
| 106737 | AA470080 | Hs.36237 | ESTs; Moderately similar to CGI-34 prote | 2.26 | LNCaP_cells, DU145_cells, MB-MDA-435s |
| 117305 | N22798 | Hs.43248 | EST | 2.26 | HT29_cells, BT474_cells, Fibroblasts 2 |
| 134470 | X54942 | Hs.83758 | CDC28 protein kinase 2 | 2.26 | DU145_cells, CALU6_cells, LNCaP_cells |
| 130734 | T99337 | Hs.18624 | KIAA1052 protein | 2.26 | Lu_AD_H23, Lu_SC_H345, Lu_SC_H69 |
| 128561 | R69227 | Hs.101489 | ESTs | 2.26 | Lu_SC_H345, DU145_cells, OVCAR_cells |
| 100670 | HG2992-H | | Beta-Hexosaminidase, Alpha Polypeptide, | 2.26 | HT29_cells, BT474_cells, Lu_SC_H345 |
| 115953 | AA443958 | Hs.90960 | ESTs | 2.26 | Caco2, 293T_cells, DU145_cells |
| 129612 | H17476 | Hs.11615 | ESTs; Highly similarto map kinase phosp | 2.25 | CALU6_cells, LNCaP_cells, PC3_cells |
| 111362 | N91973 | Hs.23595 | deoxyribonuclease III; dnaQ/mutD (E. col | 2.25 | Lu_SQ_H520, Lu_AD_H23, RPWE_2 |
| 116275 | AA485453 | Hs.250911 | interleukin 13 receptor; alpha 1 | 2.25 | OVCAR_cells, 293T_cells, DU145_cells |
| 114461 | AA024848 | Hs.126705 | ESTs | 2.25 | EB_cells, Lu_AD_H23, Lu_AD_H23 |
| 134083 | AA278393 | Hs.79013 | ESTs | 2.25 | 293T_cells, EB_cells, OVCAR_cells |
| 132470 | Z24724 | Hs.4934 | H.sapiens polyA site DNA | 2.25 | EB_cells, HS578T_cells, Caco2 |
| 114718 | AA131328 | | zo8d1.s1 Stratagene neuroepithelium NT2R | 2.25 | MB-MDA-435s, HT29_cells, Lu_SC_H69 |
| | | | SW:COX2_MOUSE P45 CYTOCHROME C OXIDASE P | | |
| 129499 | R40395 | Hs.242908 | lecithin-cholesterol acyltransferase | 2.25 | HMEC (total RNA), Fibroblasts 2, HMEC |
| 124758 | R38422 | Hs.169168 | ESTs | 2.25 | 293T_cells, RPWE_2, Lu_LC_H460 |
| 130301 | X83127 | Hs.172471 | potassium voltage-gated channel; shaker- | 2.25 | EB_cells, OVCAR_cells, A549_cells |
| 131263 | R38334 | Hs.24950 | regulator of G-protein signalling 5 | 2.25 | Lu_AD_H23, EB_cells, Lu_SC_H69 |
| 107159 | AA621340 | Hs.10600 | ESTs; Weakly similarto ORF YKR081c [S.c | 2.25 | LNCaP_cells, HMEC, EB_cells |
| 133262 | N72009 | Hs.206710 | ESTs | 2.24 | Lu_SC_H345, DU145_cells, LNCaP_cells |
| 132985 | AA093619 | Hs.62113 | KIAA0717 protein | 2.24 | EB_cells, Lu_AD_H23, Lu_AD_358 |
| 114172 | Z39043 | Hs.21421 | ESTs; Weakly similar to cysteine desulfu | 2.24 | 293T_cells, CALU6_cells, Lu_SQ_H520 |
| 127847 | AA913387 | Hs.126717 | ESTs | 2.24 | LNCaP_cells, DU145_cells, Lu_SC_H69 |
| 106499 | AA452244 | Hs.16727 | ESTs | 2.24 | Lu_SC_H345, MB-MDA-453, Lu_SC_H69 |
| 105095 | AA150088 | Hs.27023 | KIAA0917 protein | 2.24 | DU145_cells, LNCaP_cells, CALU6_cells |
| 108876 | AA134361 | Hs.191453 | ESTs | 2.24 | EB_cells, Lu_SC_H345, Lu_AD_H23 |
| 121971 | AA429667 | Hs.120405 | ESTs | 2.24 | Lu_AD_H23, 293T_cells, CALU6_cells |
| 114334 | Z41342 | Hs.22941 | ESTs | 2.24 | DU145_cells, PC3_cells, EB_cells |
| 114565 | AA063001 | Hs.103527 | SH2 domain protein 2A | 2.24 | Lu_LC_H460, MCF7, HMEC (total RNA) |
| 115766 | AA421761 | Hs.77603 | ESTs | 2.24 | Fibroblasts 2, MB-MDA-435s, MB231_cells |
| 130989 | AA608546 | Hs.21906 | ESTs | 2.24 | PC3_cells, LNCaP_cells, DU145_cells |
| 116304 | AA489461 | Hs.64742 | H sapiens mRNA for KIAA0540 protein; par | 2.24 | BT474_cells, EB₁₃ cells, LNCaP_cells |
| 111154 | N66545 | Hs.29169 | ESTs | 2.24 | OVCAR_cells, MB-MDA-435s, HMEC |
| 105561 | AA262881 | Hs.16029 | ESTs; Weakly similar to altematively sp | 2.23 | HS578T_cells, A549_cells, HMEC |
| 105939 | AA404421 | Hs.12258 | ESTs | 2.23 | EB₁₃ cells, LNCaP_cells, DU145_cells |
| 126379 | AI085342 | Hs.166146 | Homer; neuronal immediate earty gene; 3 | 2.23 | HS578T_cells, PC3_cells, RPWE_2 |
| 106610 | AA458882 | Hs.4832 | ESTs; Moderately similar to Lasp-1 prote | 2.23 | DU145_cells, MCF7, Lu_SC_H345 |
| 132786 | AA424545 | Hs.56851 | H sapiens mRNA expressed in placenta | 2.23 | EB_cells, Lu_AD_H23, Fibroblasts 2 |
| 107206 | D20728 | Hs.30767 | ESTs | 2.23 | BT474_cells, Fibroblasts 2, MB-MDA-435s |
| 133708 | R42172 | Hs.75667 | synaptophysin | 2.23 | Lu_SC_H345, CALU6_cells, Lu_SC_H69 |
| 135123 | AA227567 | Hs.9482 | target of myb 1 (chicken) homolog | 2.23 | BT474_cells, MB231_cells, EB_cells |
| 132156 | AA157401 | Hs.4113 | S-adenosylhomocysteine hydrolase-like 1 | 2.23 | DU145_cells, 293T_cells, LNCaP_cells |
| 116934 | H75624 | Hs.39662 | ESTs | 2.23 | CALU6_cells, Lu_SC_H345, Lu_LC_H460 |
| 133660 | R87373 | | ym88e05.r1 Soares aduh brain N2b4HB55Y | 2.23 | DU145_cells, A549_cells, PC3_cells |
| | | | IMAGE: 166016 5′, mRNA seq. | | |
| 119458 | W23633 | Hs.125043 | ESTs | 2.23 | 293T_cells, MB-MDA-453, OVCAR_cells |
| 101247 | L33801 | Hs.78802 | glycogen synthase kinase 3 beta | 2.23 | LNCaP_cells, EB_cells, MB-MDA-435s |
| 126008 | AA253460 | | zs06f04.s1 NCI_CGAP_GCB1 H sapiens cDNA | 2.23 | HT29_cells, PRSC_log, Fibroblasts 2 |
| 122938 | AA477119 | | zu37c7.s1 Soares ovary tumor NbHOT H sap | 2.23 | PC3_cells, MCF7, MB-MDA-434s |
| | | | TR: G288289 G288289 MITOCHONDRIAL D-LOOP | | |
| 114148 | Z38804 | Hs.184777 | ESTs; Moderately similar to OPIOID BINDI | 2.23 | HS578T_cells, Fibroblasts 2, LuSC_H345 |
| | | | MOLECULE PRECURSOR [H.sapiens] | | |
| 103433 | X98001 | Hs.78948 | Rab geranylgeranyltransferase; beta subu | 2.22 | LNCaP_cells, EB_cells, 293T_cells |
| 132954 | AA027112 | Hs.216194 | ESTs | 2.22 | EB_cells, Lu_AD_H23, Fibroblasts 2 |
| 133228 | N90029 | Hs.6831 | H sapiens clone 1400 unknown protein mRN | 2.22 | 293T_cells, PC3_cells, DU145_cells |
| 103891 | AA242887 | Hs.124186 | ring finger protein 2 | 2.22 | EB_cells, Lu_SC_H69, Lu_SC_H345 |
| 124883 | R75630 | Hs.177242 | ESTs | 2.22 | EB_cells, Lu_AD_H23, Lu_SC_H345 |
| 109921 | H05734 | Hs.30559 | ESTs | 2.22 | Lu_SQ_H520, 293T_cells, RPWE_2 |
| 127306 | AI305162 | Hs.193687 | ESTs | 2.22 | MCF7, HT29_cells, MB-MDA-453 |
| 102707 | U77456 | Hs.78103 | nucleosome assembly protein 1-like 4 | 2.22 | Caco2, EB_cells, CALU6_cells |
| 106193 | AA427625 | Hs.23272 | ESTs | 2.22 | 293T_cells, EB_cells, A549_cells |
| 118819 | N79045 | Hs.50800 | ESTs; Weakly similar to !!!! ALU SUBEAMI | 2.22 | Lu_SC_H345, LuSC_H69, DU145_cells |
| 134326 | U16306 | Hs.81800 | chondroitin sulfate proteoglycan 2 (vers | 2.22 | HS578T_cells, PRSC_log, CALU6_cells |
| 112241 | R51248 | Hs.16027 | ESTs | 2.22 | 293T_cells, HMEC (total RNA), HMEC (total RNA) |
| 123693 | AA609591 | Hs.112728 | ESTs | 2.22 | HT29_cells, HMEC (total RNA), BT474_cells |
| 129052 | AA496297 | Hs.182740 | ribosomal protein S11 | 2.22 | EB_cells, Lu_AD_H23, Lu_AD_358 |
| 122481 | AA448271 | Hs.99126 | ESTs | 2.21 | Lu_AD_H23, HT29_cells, Lu_AD_358 |
| 128895 | R37753 | Hs.106985 | ESTs | 2.21 | EB_cells, Lu_AD_H23, Lu_SC_H345 |
| 124691 | R05835 | Hs.110153 | ESTs; Weakly similar to B-CELL GROWTH FA | 2.21 | EB_cells, Lu_AD_H23, Lu_AD_358 |
| 131556 | AA442853 | Hs.2869 | cyclin-dependent kinase 5; regulatory su | 2.21 | HT29_cells, Lu_LC_H460, Lu_SC_H69 |
| 128869 | AA424570 | Hs.106736 | ESTs | 2.21 | EB_cells, Lu_AD_H23, Lu_SC_H69 |
| 107114 | AA610089 | Hs.11776 | U4/U6-associated RNA splicing factor | 2.21 | MCF7, Lu_SC_H345, DU145_cells |
| 106255 | AA431191 | Hs.161489 | ESTs | 2.21 | EB_cells, Caco2, DU145_cells |
| 130724 | AA370091 | Hs.179680 | ESTs | 2.2 | EB_cells, Lu_AD_H23, Lu_SC_H69 |
| 105483 | AA255874 | Hs.23458 | ESTs | 2.2 | LNCaP_cells, DU145_cells, PC3_cells |
| 118970 | N93503 | Hs.54961 | stoned B/TFIIA-alpha/beta like factor | 2.2 | 293T_cells, HS578T_cells, OVCAR_cells |
| 120805 | AA346041 | Hs.96844 | ESTs | 2.2 | HT29_cells, HS578T_cells, 293T_cells |
| 106158 | AA425382 | Hs.6553 | ESTs | 2.2 | CALU6_cells, PC3_cells, EB_cells |
| 102121 | U14391 | Hs.82251 | myosin IC | 2.2 | A549_cells, EB_cells, Caco2 |
| 109446 | AA232125 | Hs.87062 | ESTs | 2.2 | HT29_cells, Lu_LC_H460, CALU6_cells |
| 129515 | AA490882 | Hs.112227 | ESTs | 2.2 | Lu_SC_H345, BT474_cells, Caco2 |
| 113128 | T49325 | Hs.8977 | ESTs | 2.2 | Lu_SQ_H520, Lu_AD_H23, Lu_AD_358 |
| 127289 | AI041014 | Hs.220752 | ESTs | 2.2 | EB_cells, Lu_AD_H23, Lu_AD_H23 |
| 129912 | AA047344 | Hs.107213 | ESTs; Highly similar to NY-REN-6 antigen | 2.2 | CALU6_cells, A549_cells, EB_cells |
| 115700 | AA411655 | Hs.67709 | ESTs | 2.2 | OVCAR_cells, EB_cells, Caco2 |
| 106267 | AA431873 | Hs.4988 | H sapiens clone 24711 mRNA seq | 2.2 | Lu_SQ_H520, EB_cells, PC3_cells |
| 112881 | T03593 | Hs.182814 | ESTs | 2.19 | A549_cells, OVCAR_cells, 293T_cells |
| 116902 | H70739 | | yu69f11.s1 Weizmann Olfactory Epithelium | 2.19 | LNCaP_cells, DU145_cells, PC3_cells |
| | | | IMAGE: 239085 3′ similar to contains LTR | | |
| 105621 | AA280865 | Hs.6375 | H sapiens mRNA; cDNA DKFZp564K0222 (from | 2.19 | HMEC, Caco2, HMEC (total RNA) |
| 126991 | R31652 | Hs.821 | biglycan | 2.19 | Fibroblasts 2, LuSC_H69, HS578T_cells |
| 125466 | R08234 | Hs.180461 | ESTs | 2.19 | Lu_AD_358, Lu_AD_H23, Lu_SQ_H520 |
| 108491 | AA082973 | | zn7g1.s1 Stratagene hNT neuron (#937233) | 2.19 | Lu_AD_358, RPWE_2, Lu_LC_H460 |
| | | | to gb:M3672 6S RIBOSOMAL PROTEIN L7A (H | | |
| 109978 | H09356 | Hs.22528 | ESTs | 2.19 | PRSC_log, Lu_SC_H345, Lu_SC_H69 |
| 106990 | AA521354 | Hs.24758 | ESTs | 2.19 | EB_cells, LNCaP_cells, OVCAR_cells |
| 122362 | AA443919 | Hs.96840 | ESTs | 2.19 | EB_cells, Lu_AD_358, PRSC_con |
| 125367 | AI016490 | Hs.81964 | SEC24 (S. cerevisiae) related gene famil | 2.19 | HT29_cells, Lu_SC_H69, Lu_AD_H23 |
| 110716 | H97188 | Hs.35096 | ESTs | 2.19 | DU145_cells, Fibroblasts 2, PRSC_con |
| 129297 | R11267 | Hs.180570 | H sapiens chromosome 19; cosmid F22329 | 2.19 | 293T_cells, MB-MDA-435s, A549_cells |
| 104992 | AA102652 | Hs.22753 | ESTs; Weakly similar to coded for by C. | 2.18 | MCF7, MB-MDA-453, Lu_SQ_H520 |
| 119896 | W84738 | Hs.137319 | ESTs | 2.18 | 293T_cells, 293T_cells, OVCAR_cells |
| 118594 | N69022 | Hs.49599 | ESTs | 2.18 | Lu_SC_H69, Lu_AD_H23, Lu_SC_H345 |
| 129786 | H98977 | Hs.246109 | ESTs | 2.18 | 293T_cells, 293T_cells, 293T_cells |
| 104325 | D81608 | Hs.150675 | polymerase (RNA) II (DNA directed) polyp | 2.18 | PC3_cells, Lu_SC_H345, LNCaP_cells |
| 123022 | AA480909 | | aa28f10.s1 NCI_CGAP_GCB1 H sapiens cDNA | 2.18 | OVCAR_cells, DU145_cells, LNCaP_cells |
| | | | Alu repetitive element; contains element | | |
| 133572 | W94333 | Hs.7499 | translocase of inner mitochondrial membr | 2.18 | Caco2, LNCaP_cells, Lu_SQ_H520 |
| 133363 | AA479713 | Hs.71962 | ESTs | 2.18 | EB_cells, Lu_AD_H23, Fibroblasts 2 |
| 135361 | AA053319 | Hs.167700 | ESTs | 2.18 | EB_cells, 293T_cells, Caco2 |
| 128319 | AA808904 | Hs.115095 | ESTs; Weakly similar to RHO-RELATED GTP- | 2.18 | Lu_SC_H345, OVCAR_cells, DU145_cells |
| 128660 | AA011597 | Hs.177398 | ESTs | 2.18 | EB_cells, Lu_AD_H23, Lu_SQ_H520 |
| 114877 | AA235618 | Hs.205125 | ESTs | 2.18 | DU145_cells, 293T_cells, OVCAR_cells |
| 125925 | H28737 | | ESTs; Moderately similar to !!!! ALU SUB | 2.18 | Lu_SC_H69, Lu_SC_H345, HS578T_cells |
| 113427 | T85105 | Hs.15471 | ESTs | 2.18 | EB_cells, Lu_AD_H23, Lu_SC_H69 |
| 117500 | N31909 | Hs.44278 | ESTs | 2.18 | PRSC_con, Lu_SC_H345, PRSC_log |
| 131384 | F13608 | Hs.26226 | ESTs | 2.18 | 293T_cells, LNCaP_cells, OVCAR_cells |
| 134499 | U70370 | Hs.84136 | paired-like homeodomain transcnption fa | 2.18 | Caco2, BT474_cells, MB231_cells |
| 128154 | AA922969 | Hs.127100 | ESTs | 2.17 | MB-MDA-453, MB-MDA-453, Lu_SC_H345 |
| 134585 | T48154 | Hs.168655 | H sapiens mRNA for H-2K binding factor-2 | 2.17 | LNCaP_cells, 293T_cells, PRSC_log |
| 104987 | AA101723 | Hs.16683 | ESTs | 2.17 | EB_cells, MCF7, DU145_cells |
| 132992 | AA091017 | Hs.6226 | ESTs | 2.17 | Caco2, LNCaP_cells, DU145_cells |
| 135311 | M36089 | Hs.98493 | X-ray repair complementing defective rep | 2.17 | HMEC (total RNA), Fibroblasts 2, HMEC |
| 113171 | T54613 | Hs.9761 | EST | 2.17 | HT29_cells, PRSC_con, Lu_SQ_H520 |
| 117736 | N46999 | Hs.46648 | ESTs | 2.16 | PRSC_log, OVCAR_cells, A549_cells |
| 125181 | W58461 | Hs.12396 | ESTs | 2.16 | LNCaP_cells, DU145_cells, 293T_cells |
| 120187 | Z40251 | Hs.56974 | ESTs | 2.16 | LNCaP_cells, MB-MDA-453, HMEC (total RNA) |
| 100308 | D50532 | Hs.54403 | macrophage lectin 2 (calcium dependent) | 2.16 | HT29_cells, Lu_AD_H23, Lu_AD_H23 |
| 110960 | N50887 | Hs.26549 | ESTs; Weakly similar to KIAA0449 protein | 2.16 | Caco2, A549_cells, LNCaP_cells |
| 113608 | T93113 | | ESTs; Moderately similar to !!!! ALU SUB | 2.16 | Lu_SC_H69, CALU6_cells, 293T_cells |
| 107538 | Z21089 | Hs.50094 | ESTs; Weakly similar to KALIRIN [R.norve | 2.16 | HS578T_cells, 293T_cells, DU145_cells |
| 128703 | S76992 | Hs.104005 | vav 2 oncogene | 2.16 | RPWE_2, Lu_SC_H69, HT29_cells |
| 126065 | AI366484 | | ESTs | 2.16 | 293T_cells, CALU6_cells, A549_cells |
| 130000 | AA465727 | Hs.124084 | ESTs; Weakly similar to !!!! ALU SUBFAMI | 2.16 | DU145_cells, LNCaP_cells, OVCAR_cells |
| 120407 | AA235040 | Hs.107283 | ESTs | 2.16 | EB_cells, 293T_cells, A549_cells |
| 121199 | AA400371 | Hs.97792 | ESTs | 2.16 | Lu_AD_358, Lu_AD_H23, A549_cells |
| 114963 | AA243867 | Hs.193055 | ESTs | 2.16 | DU145_cells, PRSC_con, LNCaP_cells |
| 100343 | D63874 | Hs.189509 | high-mobility group (nonhistone chromoso | 2.15 | CALU6_cells, MB-MDA-453, Caco2 |
| 125077 | T88822 | | yd32f5.s1 Soares fetal liver spleen 1NFL | 2.15 | Lu_AD_H23, Lu_SC_H69, Lu_SC_H345 |
| 117286 | N22181 | | yw36d12.s1 Morton Fetal Cochlea H sapien | 2.15 | 293T_cells, Lu_Sc_H345, Lu_SC_H69 |
| 132876 | AA130603 | Hs.169683 | ESTs; Moderately similarto !!!! ALU SUB | 2.15 | EB_cells, LNCaP_cells, HS578T_cells |
| 133834 | AA147510 | Hs.154737 | serine protease; umbilical endothelium | 2.15 | DU145_cells, EB_cells, Caco2 |
| 126908 | AA169866 | | ESTs; Weakly similar to !!!! ALU SUBFAMI | 2.15 | DU145_cells, LNCaP_cells, OVCAR_cells |
| 106900 | AA490142 | Hs.6193 | ESTs | 2.15 | Fibroblasts 2, Lu_AD_H23, PRSC_con |
| 129398 | AA437374 | Hs.234573 | H sapiens mRNA for TL132 | 2.15 | MCF7, DU145_cells, LNCaP_cells |
| 114512 | AA044274 | Hs.165215 | ESTs | 2.15 | Lu_AD_358, MB-MDA-453, HS578T_cells |
| 134381 | U56637 | Hs.184270 | capping protein (actin filament) muscle | 2.15 | LNCaP_cells, EB_cells, PC3_cells |
| 118843 | N80671 | Hs.220255 | ESTs | 2.14 | EB_cells, DU145_cells, MCF7 |
| 115526 | AA342049 | Hs.69606 | ESTs | 2.14 | 293T_cells, Caco2, Lu_SC_H69 |
| 123460 | AA598981 | Hs.251122 | EST | 2.14 | Lu_SC_H345, DU145_cells, MCF7 |
| 119812 | W73951 | Hs.58348 | ESTs; Weakly similar to CORNIFIN A [H.sa | 2.14 | 293T_cells, HS578T_cells, CALU6_cells |
| 105263 | AA227926 | Hs.6682 | ESTs | 2.14 | A549_cells, HMEC (total RNA), EB_cells |
| 129242 | W81679 | Hs.5174 | ribosomal protein S17 | 2.14 | 293T_cells, CALU6_cells, HMEC (total RNA) |
| 132348 | AA037285 | Hs.170311 | heterogeneous nuclear ribonucleoprotein | 2.14 | A549_cells, HT29_cells, Lu_SQ_H520 |
| 114425 | AA015763 | Hs.132812 | ESTs | 2.14 | 293T_cells, HS578T_cells, PRSC_con |
| 127759 | AI369384 | | arylsulfatase D | 2.14 | DU145_cells, LNCaP_cells, EB_cells |
| 134069 | U29607 | Hs.78935 | methionine aminopeplidase; elF-2-associa | 2.14 | Lu_SC_H345, DU145_cells, MCF7 |
| 116158 | AA461187 | Hs.61762 | ESTs | 2.14 | Lu_SC_H69, MCF7, MB-MDA-453 |
| 125627 | R35166 | Hs.14881 | ESTs | 2.14 | HT29_cells, Fibroblasts 2, BT474_cells |
| 118684 | N71364 | Hs.109510 | ESTs | 2.14 | OVCAR_cells, PRSC_con, HS578T_cells |
| 119419 | T97977 | Hs.60260 | ESTs | 2.14 | Lu_AD_H23, Lu_SQ_H520, Lu_SQ_H520 |
| 133097 | N67515 | Hs.6479 | ESTs; Weakly similar to KIAA0872 protein | 2.14 | EB_cells, Lu_AD_H23, Lu_AD_358 |
| 112121 | R45445 | Hs.252723 | H sapiens mRNA; cDNA DKFZp434D115 (from | 2.13 | Lu_AD_H23, Lu_AD_358, BT474_cells |
| 114894 | AA236019 | Hs.188803 | ESTs | 2.13 | MB-MDA-453, MCF7, Lu_SQ_H520 |
| 124087 | H08773 | | y194d5.s1 Soares infant brain 1NIB H sap | 2.13 | Lu_SC_H69, Fibroblasts 2, HMEC (total RNA) |
| 111902 | R39191 | Hs.109445 | KIAA1020 protein | 2.13 | Caco2, 293T_cells, Lu_SC_H69 |
| 119943 | W86835 | Hs.14158 | copine III | 2.13 | LNCaP_cells, PC3_cells, HS578T_cells |
| 109276 | AA196306 | Hs.86045 | ESTs | 2.13 | Lu_SC_H345, Lu_SC_H69, Lu_LC_H460 |
| 117351 | N24581 | Hs.43230 | ESTs | 2.13 | HS578T_cells, CALU6_cells, PRSC_con |
| 116046 | AA453461 | Hs.94491 | H sapiens clone 23585 mRNA seq | 2.13 | LNCaP_cells, Caco2, EB_cells |
| 112785 | R96478 | Hs.16586 | ESTs | 2.13 | EB₁₃ cells, Lu_AD_H23, Lu_SC_H69 |
| 115835 | AA428576 | Hs.41371 | ESTs | 2.13 | EB_cells, Lu_SC_H345, OVCAR_cells |
| 127499 | T49891 | Hs.119252 | tumor protein; translationally-controlle | 2.13 | EB_cells, PRSC_con, LNCaP_cells |
| 129951 | AA019475 | Hs.74615 | platelet-derived growth factor receptor, | 2.13 | EB_cells, Lu_AD_H23, Lu_SC_H69 |
| 124270 | H79560 | Hs.107840 | ESTs | 2.13 | OVCAR_cells, 293T_cells, 293T_cells |
| 133766 | D52420 | Hs.184326 | cell division cycle 10 (homologous to CD | 2.12 | CALU6_cells, DU145_cells, PC3_cells |
| 109248 | AA194720 | Hs.189996 | ESTs; Highly similar to sec61 homolog [H | 2.12 | HT29_cells, MB231_cells, HMEC (total RNA) |
| 106724 | AA465226 | Hs.28631 | ESTs | 2.12 | EB_cells, 293T_cells, DU145_cells |
| 100571 | HG2264-H | | Atpase, Ca2+ Transporting, Plasma Membra | 2.12 | EB_cells, Lu_AD_H23, Lu_SC_H69 |
| 133017 | AA450187 | Hs.178518 | ESTs | 2.12 | OVCAR_cells, PC3_cells, 293T_cells |
| 124313 | H94650 | Hs.108002 | ESTs | 2.12 | MB-MDA-453, Lu_SC_H345, HT29_cells |
| 113059 | T26925 | Hs.172684 | vesicle-associated membrane protein 8 (e | 2.12 | MB-MDA-453, PC3_cells, LNCaP_cells |
| 113241 | T63313 | Hs.226136 | ESTs; Moderately similar to !!!! ALU SUB | 2.12 | HMEC (total RNA), BT474_cells, HMEC |
| 111952 | R40782 | Hs.21296 | ESTs | 2.12 | HT29_cells, PC3_cells, A549_cells |
| 113965 | W86519 | Hs.19631 | ESTs | 2.12 | PC3_cells, EB_cells, LNCaP_cells |
| 108059 | AA043944 | Hs.62663 | ESTs | 2.12 | EB_cells, OVCAR_cells, 293T_cells |
| 124235 | H63994 | Hs.221134 | ESTs | 2.12 | Fibroblasts 2, MB-MDA-453, PRSC_con |
| 106400 | AA447621 | Hs.31257 | ESTs | 2.12 | DU145_cells, EB_cells, Caco2 |
| 119590 | W44798 | Hs.55876 | ESTs | 2.12 | PRSC_log, Lu_SC_H69, Lu_SC_H345 |
| 112434 | R63068 | Hs.159793 | EST | 2.11 | HS578T_cells, LNCaP_cells, OVCAR_cells |
| 122731 | AA457549 | | aa92b1.s1 Stratagene fetal retina 93722 | 2.11 | MB-MDA-453, RPWE_2, MCF7 |
| | | | gb:X5275_ma3 LEUKOSIALIN PRECURSOR (HU | | |
| 115348 | AA281562 | Hs.88860 | ESTs | 2.11 | EB_cells, Lu_AD_H23, Fibroblasts 2 |
| 128873 | AA226768 | Hs.109483 | ESTs; Weakly similar to predicted using | 2.11 | MB-MDA-435s, EB_cells, LNCaP_cells |
| 133742 | T54301 | Hs.75844 | ESTs | 2.11 | EB_cells, CALU6_cells, DU145_cells |
| 102099 | U11870 | Hs.194778 | interleukln 8 receptor alpha | 2.11 | Lu_AD358, PC3_cells, PRSC_con |
| 125840 | H05787 | Hs.12064 | ubiquitin specific protease 22 | 2.11 | EB_cells, LNCaP_celis, Caco2 |
| 106501 | AA256604 | Hs.31930 | ESTs | 2.1 | Fibroblasts 2, HS578T_cells, MB-MDA-4355 |
| 111576 | R10334 | Hs.15489 | ESTs | 2.1 | Lu_SC_H69, PRSC_log, Lu_SC_H345 |
| 104275 | C02170 | Hs.39387 | ESTs; Weakly similar to weak similarity | 2.1 | HT29_cells, MB231_cells, Lu_SC_H69 |
| 117803 | N48620 | Hs.28483 | pregnancy specific beta-1-glycoprotein 9 | 2.1 | HT29_cells, HMEC, RPWE_2 |
| 122725 | AA457407 | Hs.152204 | transmembrane protease; serine 2 | 2.1 | Lu_SC_H69, Lu_LC_H450, Lu_SC_H345 |
| 120987 | AA398233 | Hs.111894 | KIAA0108 gene product | 2.1 | Fibroblasts 2, PRSC_con, MCF7 |
| 105932 | AA403305 | Hs.12185 | ESTs; Weakly similar to myosin phosphata | 2.1 | LNCaP_cells, MCF7, OVCAR_cells |
| 118398 | N64706 | Hs.137282 | ESTs | 2.1 | Lu_SC_H345, HT29_cells, HMEC |
| 103679 | Z86000 | | Human DNA seq from PAC 151B14 onchromos | 2.1 | CALU6_cells, A549_cells, Lu_SC_H345 |
| | | | receptor subtype 3 (SSTR3), tRNA, ESTs, | | |
| 130303 | L40392 | Hs.180789 | H sapiens (clone S164) mRNA; 3′ end of c | 2.1 | PC3_cells, DU145_cells, LNCaP_cells |
| 122815 | AA461080 | Hs.139446 | ESTs | 2.1 | HT29_cells, BT474_cells, MB231_cells |
| 105598 | AA279439 | Hs.20594 | ESTs; Weakly similar to misato [D.melano | 2.1 | EB₁₃ cells, Lu_SC_H345, LNCaP_cells |
| 124889 | R69088 | Hs.28728 | ESTs; Weakly similar to F55A12.9 [C.eleg | 2.1 | HT_cells, BT474_cells, MB231_cells |
| 129599 | F10720 | Hs.180804 | ESTs | 2.1 | HS578T_cells, HT29_cells, HT29_cells |
| 110338 | H40359 | Hs.177256 | ESTs | 2.09 | MCF7, A549_cells, MB-MDA-435s |
| 134092 | H17490 | Hs.7905 | ESTs: Highly similar to sorting nexin 9 | 2.09 | EB_cells, Fibroblasts 2 HS578T_cells |
| 133002 | AF006082 | Hs.62461 | ARP2 (actin-related protein 2; yeast) ho | 2.09 | EB_cells, HS578T_cells, A549_cells |
| 115570 | AA398343 | Hs.94943 | ESTs | 2.09 | Lu_SC_H345, PC3_cells, LNCaP_cells |
| 120055 | W93299 | Hs.59363 | ESTs; Weakly similar to cytokeratin 20 [ | 2.09 | HMEC (total RNA), HS578T_cells, HS578T_cells |
| 116332 | AA491208 | Hs.62620 | ESTs | 2.09 | EB_cells, Lu_AD_H23, Lu_SC_H69 |
| 105415 | AA243768 | Hs.4232 | ESTs; Highly similar to match to ESTs Z4 | 2.09 | LNCaP_cells, Lu_AD_H23, MB-MDA-453 |
| 116607 | D80354 | Hs.256321 | EST | 2.09 | LNCaP_cells, DU145_cells, RPWE_2 |
| 126731 | AA593973 | Hs.232217 | ESTs; Weakly similar to !!!! ALU SUBFAMI | 2.09 | MB231_cells, HT29_cells, HMEC |
| 102276 | U30999 | Hs.10247 | activated leucocyte cell adhesion molecu | 2.09 | PC3_cells, HS578T_cells, DU145_cells |
| 113666 | T96077 | Hs.17738 | EST | 2.09 | Lu_AD_H23, Lu_AD_H23, Lu_SQ_H520 |
| 101183 | L19779 | Hs.795 | H2A histone family; member O | 2.09 | LNCaP_cells, MCF7, OVCAR_cells |
| 112177 | R49025 | Hs.22996 | ESTs | 2.09 | Lu_AD_H23, Lu_AD_358, Lu_SC_H69 |
| 115038 | AA252360 | Hs.87968 | ESTs | 2.08 | BT474_cells, MB231_cells, HT29_cells |
| 109638 | F04432 | Hs.17904 | ESTs | 2.08 | EB_cells, DU145_cells, PC3_cells |
| 109592 | F02475 | Hs.26370 | ESTs | 2.08 | Lu_AD_H23, Lu_SQ_H520, Lu_LC_H460 |
| 133740 | U68142 | Hs.170160 | RAB2: member RAS oncogene family-like | 2.08 | LNCaP_cells, MB-MDA-453, EB_cells |
| 126716 | AA031700 | Hs.251962 | ESTs | 2.08 | HS578T_cells, Fibroblasts 2, Lu_SC_H69 |
| 124055 | F10904 | Hs.100516 | H sapiens clone 23605 mRNA seq | 2.08 | Lu_SC_H345, OVCAR_cells, DU145_cells |
| 113283 | T66813 | Hs.12947 | EST | 2.08 | EB_cells, Lu_SC_H69, Lu_AD_H23 |
| 120097 | W95068 | Hs.59621 | ESTs | 2.08 | HS578T_cells, A549_cells, CALU6_cells |
| 102066 | U08471 | Hs.352 | folate receptor 3 (gamma) | 2.08 | EB_cells, Lu_AD_H23, Lu_AD_358 |
| 108712 | AA121993 | | zm24d11.s1 Stratagene pancreas (#93728) | 2.08 | Lu_SQ_H520, HT29_cells, BT474_cells |
| | | | similar to gb:Y433 GLUTATHIONE PEROXIDAS | | |
| 134453 | X70683 | Hs.83484 | SRY (sex determining region Y)-box 4 | 2.08 | EB_cells, Lu_SC_H345, Lu_SC_H69 |
| 103883 | AA232836 | Hs.87363 | ESTs | 2.08 | HT29_cells, 293T_cells, 293T_cells |
| 105313 | AA233856 | Hs.16930 | ESTs | 2.08 | DU145_cells, MB-MDA-435s, HS578T_cells |
| 113669 | T96148 | Hs.17762 | ESTs | 2.08 | EB_cells, Lu_SQ_H520, Fibroblasts 2 |
| 120380 | AA227904 | Hs.104223 | ESTs | 2.08 | 293T_cells, CALU6_cells, A549_cells |
| 121045 | AA398554 | Hs.181012 | double-stranded RNA-binding zinc finger | 2.08 | 293T_cells, PC3_cells, OVCAR_cells |
| 104949 | AA070735 | Hs.148090 | ESTs | 2.08 | Lu_SC_H69, Lu_SC_H345, RPWE_2 |
| 118751 | N74210 | Hs.50454 | EST | 2.08 | Lu_AD_H23, Lu_SC_H69, Lu_SC_H345 |
| 112399 | R60920 | Hs.26419 | H sapiens clone 24510 mRNA seq | 2.08 | EB_cells, Lu_AD_H23, Lu_SC_H69 |
| 129994 | AA599443 | Hs.38194 | ESTs; Moderately similar to !!!! ALU SUB | 2.08 | DU145_cells, EB_cells, HS578T_cells |
| 116402 | AA600054 | Hs.65302 | ESTs | 2.08 | HT29_cells, BT474_cells, Lu_AD_H23 |
| 125307 | Z40583 | Hs.101259 | ESTs | 2.08 | HMEC, HMEC (total RNA), EB_cells |
| 105047 | AA132453 | Hs.15396 | ESTs | 2.08 | Caco2, HT29_cells, LNCaP_cells |
| 128659 | T95280 | Hs.103315 | trinucleotide repeat containing 1 | 2.08 | EB_cells, Lu_AD_H23, Lu_SC_H69 |
| 122301 | AA437378 | Hs.98791 | ESTs | 2.08 | Lu_SC_H345, Lu_AD_H23, Lu_AD_358 |
| 121974 | AA429804 | Hs.229675 | EST | 2.08 | HS578T_cells, 293T_cells, OVCAR_cells |
| 116905 | H71420 | | ys8c12.s1 Soares fetal liver spleen 1NFL | 2.08 | Lu_AD_H23, EB_cells, PRSC_con |
| | | | 3′ similar to contains Alu repetitive e | | |
| 106703 | AA463979 | Hs.21264 | KIAA0782 protein | 2.08 | EB_cells, Caco2, PRSC_con |
| 121908 | AA427858 | Hs.98534 | EST | 2.07 | 293T_cells, Lu_SC_H345, CALU6_cells |
| 135119 | T23992 | Hs.94769 | ESTs; Moderately similar to RAS-RELATED | 2.07 | HS578T_cells, PRSC_con, OVCAR_cells |
| 103558 | Z19574 | Hs.2785 | keratin 17 | 2.07 | RPWE_2, HMEC (total RNA), HMEC |
| 124209 | H57317 | Hs.193433 | ESTs | 2.07 | Fibroblasts 2, OVCAR_cells, 293T_cells |
| 133936 | AA045083 | Hs.77719 | gamma-glutamyl carboxylase | 2.07 | Fibroblasts 2, MB-MDA-453, PRSC_con |
| 116246 | AA479961 | Hs.42913 | ESTs; Highly similar to ubiquilin-conjug | 2.07 | EB_cells, LNCaP_cells, LNCaP_cells |
| 123230 | AA490134 | Hs.105308 | EST | 2.07 | Lu_AD_H23, Lu_SC_H69, Lu_SC_H345 |
| 127378 | AA452696 | | zx39b05.r1 Soares_total_fetus_Nb2HF8_9w | 2.07 | HS578T_cells, LNCaP_cells, EB_cells |
| | | | to contains Alu repetitive element; cont | | |
| 110464 | H53013 | Hs.221901 | ESTs | 2.07 | Fibroblasts 2, Lu_SQ_H520, Lu_SQ_H520 |
| 135191 | X07619 | Hs.169876 | cytochrome P450; subfamily IID (debrisoq | 2.07 | Lu_AD_H23, Lu_SC_H69, Lu_AD_358 |
| | | | polypeptide 7a (pseudogene) | | |
| 101267 | L36818 | Hs.75339 | inositol polyphosphate phosphatase-like | 2.07 | Lu_SC_H345, OVCAR_cells, Caco2 |
| 105185 | AA191495 | Hs.189937 | ESTs | 2.07 | Lu_SC_H69, Lu_AD_H23, Lu_SC_H345 |
| 125366 | H60192 | Hs.76853 | ESTs; Weakly similar to human homolog of | 2.07 | DU145_cells, Lu_LC_H460, Lu_AD_358 |
| 117472 | N30131 | Hs.93738 | DKFZP434M098 protein | 2.07 | EB_cells, Lu_SC_H69, 293T_cells |
| 114235 | Z39710 | Hs.25341 | ESTs | 2.07 | DU145_cells, BT474_cells, Lu_SC_H69 |
| 109081 | AA165268 | Hs.72488 | ESTs | 2.07 | Lu_SC_H69, Lu_SC_H345, PC3_cells |
| 112596 | R78212 | Hs.163705 | ESTs | 2.07 | MB-MDA-435s, Lu_SQ_H520, MB-MDA-453 |
| 109254 | AA194940 | Hs.85956 | ESTs; Weakly similar to line-1 protein O | 2.07 | HS578T_cells, 293T_cells, OVCAR_cells |
| 105898 | AA401144 | Hs.27354 | ESTs | 2.07 | EB_cells, 293T_cells, PRSC_con |
| 116290 | AA488691 | Hs.57969 | phenylalanine-tRNA synthetase | 2.06 | Lu_AD_H23, Lu_SC_H345, PRSC_log |
| 122529 | AA449828 | Hs.99229 | ESTs | 2.06 | DU145_cells, HS578T_cells, 293T_cells |
| 104612 | R99199 | Hs.173063 | transducin-like enhancer of split 2: hom | 2.06 | MB-MDA-435s, 293T_cells, 293T_cells |
| 116465 | AA621650 | Hs.41045 | ESTs; Weakly similar to KIAA0734 protein | 2.06 | MB231_cells, HT29_cells, Lu_AD_358 |
| 123155 | AA488414 | Hs.76127 | hect (homologous to the E6-AP (UBE3A) ca | 2.06 | DU145_cells, CALU6_cells, PC3_cells |
| | | | domain (RLD) 1 | | |
| 126752 | AI073373 | Hs.183275 | ESTs | 2.06 | LNCaP_cells, EB_cells, DU145_cells |
| 126455 | N80749 | Hs.111515 | ESTs; Weakly similar to predicted using | 2.06 | CALU6_cells, PRSC_log, OVCAR_cells |
| 129339 | R77869 | Hs.28506 | ESTs | 2.06 | EB_cells, BT474_cells, Lu_AD_H23 |
| 115021 | AA252028 | Hs.39168 | ESTs | 2.06 | Lu_SQ_H520, Fibroblasts 2, EB_cells |
| 129054 | T67231 | Hs.168289 | succinate dehydrogenase complex; subunit | 2.06 | Caco2, LNCaP_cells, EB_cells |
| 101261 | L35545 | Hs.82353 | endothelial cell protein C/activated pro | 2.06 | EB_cells, RPWE_2, DU145_cells |
| 132697 | AA281951 | Hs.5518 | H sapiens mRNA; cDNA DKFZp566J2146 (from | 2.06 | OVCAR_cells, LNCaP_cells, DU145_cells |
| 124380 | N26536 | Hs.84999 | ATPase; Cu++ transporting; beta polypept | 2.06 | Caco2, Caco2, 293T_cells |
| 103967 | AA303711 | Hs.144700 | ephrin-B1 | 2.06 | HT29_cells, HMEC (total RNA), HMEC |
| 119403 | T92935 | Hs.119908 | ESTs; Highly similarto nucleolar protei | 2.06 | HMEC, EB_cells, HMEC (total RNA) |
| 125755 | R66080 | Hs.191268 | H sapiens mRNA; cDNA DKFZp434N174 (from | 2.06 | LNCaP_cells, DU145_cells, OVCAR_cells |
| 101843 | M93405 | Hs.170008 | methylmalonate-semialdehyde dehydrogenas | 2.05 | LNCaP_cells, MB-MDA-453, EB_cells |
| 113032 | T24024 | Hs.7387 | DKFZPS64B116 protein | 2.05 | EB_cells, A549_cells, A549_cells |
| 112563 | R72632 | Hs.29282 | ESTs | 2.05 | MCF7, HS578T_cells, PRSC_con |
| 126432 | AA583825 | Hs.235860 | ESTs | 2.05 | MB231_cells, HT29_cells, Fibroblasts 2 |
| 101636 | M57763 | Hs.89474 | ADP-ribosylation factor 6 | 2.05 | DU145_cells, LNCaP_cells, PC3_cells |
| 125174 | W51835 | Hs.231082 | EST | 2.05 | 26_cells, Fibroblasts 2, Lu_AD_H23 |
| 106168 | AA425943 | Hs.82208 | acyl-Coenzyme A dehydrogenase; very long | 2.05 | OVCAR_cells, PC3_cells, EB_cells |
| 135343 | AA236796 | Hs.9914 | follistatin | 2.05 | HMEC (total RNA), PC3_cells, HMEC |
| 105267 | AA227956 | Hs.25348 | follistatin-like 3 (secreted glycoprotel | 2.05 | HMEC, RPWE_2, HMEC (total RNA) |
| 134331 | AA452020 | Hs.234156 | ESTs; Weakly similar to CGI-128 protein | 2.05 | EB_cells, CALU6_cells, A549_cells |
| 121634 | AA417012 | Hs.28921 | ESTs | 2.05 | HS578T_cells, EB_cells, Lu_SC_H345 |
| 131394 | R72637 | Hs.26343 | ESTs | 2.05 | EB_cells, Lu_SC_H69, Lu_AD_H23 |
| 111526 | R05260 | Hs.20131 | ESTs | 2.05 | Lu_AD_H23, Lu_SC_H69, BT474_cells |
| 125049 | T79840 | Hs.111798 | ESTs | 2.05 | HT29_cells, Lu_AD_H23, Lu_SC_H345 |
| 120433 | AA237077 | Hs.180777 | H sapiens mRNA; cDNA DKFZp564M0264 (from | 2.05 | DU145_cells, CALU6_cells, PC3_cells |
| 129498 | AA449789 | Hs.75511 | connective tissue growth factor | 2.05 | HS578T_cells, PRSC_log, PRSC_con |
| 127805 | AA740921 | Hs.1197 | heat shock 10 kD protein 1 (chaperonin 10 | 2.05 | DU145_cells, LNCaP_cells, OVCAR_cells |
| 109275 | AA196287 | Hs.20303 | ESTs; Moderately similar to !!!! ALU SUB | 2.05 | EB_cells, MB-MDA-453, Fibroblasts 2 |
| 120683 | AA290987 | Hs.49657 | ESTs; Weakly similar to contains similar | 2.04 | Lu_AD_358, Lu_SQ_H520, Lu_LC_H460 |
| 135415 | X60655 | Hs.99967 | even-skipped homeo box 1 (homolog of Dro | 2.04 | Lu_AD_H23, RPWE_2, Lu_SQ_H520 |
| 132925 | AA252759 | Hs.238296 | DKFZP434A033 protein | 2.04 | 293T_cells, HS578T_cells, LNCaP_cells |
| 101875 | M97287 | Hs.74592 | special AT-rich seq binding protein 1 (b | 2.04 | EB_cells, Lu_SC_H69, 293T_cells |
| 101453 | M22490 | Hs.65879 | bone morphogenetic protein 4 | 2.04 | PRSC_con, HT29_cells, MB231_cells |
| 129177 | T95005 | Hs.209587 | ESTs | 2.04 | 293T_cells, MB-MDA-435s, Lu_SC_H69 |
| 130726 | W55946 | Hs.18508 | putative glycine-N-acyltransferase | 2.04 | HT29_cells, Fibroblasts 2, MB-MDA-435s |
| 105549 | AA262417 | Hs.5415 | ESTs | 2.04 | DU145_cells, OVCAR_cells, PC3_cells |
| 124543 | N63706 | Hs.104573 | ESTs | 2.04 | Caco2, 293T_cells, DU145_cells |
| 123062 | AA482069 | Hs.100847 | ESTs | 2.04 | Lu_AD_358, HT29_cells, HT29_cells |
| 109464 | AA232557 | Hs.87100 | ESTs | 2.04 | DU145_cells, Lu_AD_H23, LNCaP_cells |
| 129619 | AA610116 | Hs.11663 | tetraspan NET-6 protein | 2.04 | BT474_cells, Caco2, LNCaP_cells |
| 127545 | AA935809 | Hs.115899 | ESTs | 2.04 | BT474_cells, MB-MDA-4355, MB-MDA-453 |
| 133068 | R73427 | Hs.235712 | ESTs | 2.04 | Caco2, OVCAR_cells, MCF7 |
| 113609 | T93263 | Hs.16875 | ESTs; Weakly similar to hypothetical pro | 2.04 | EB_cells, Lu_SC_H345, PRSC_con |
| 106645 | AA460270 | Hs.27695 | midline 1 (Opitz/BBB syndrome) | 2.04 | A549_cells, 293T_cells, Caco2 |
| 126256 | Z21124 | | HSAAADNVE TEST1, Human adult Testis tiss | 2.04 | Fibroblasts 2, Fibroblasts 2, MCF7 |
| 129697 | R00541 | Hs.172069 | DKFZP434C212 protein | 2.04 | HT29_cells, Lu_SQ_H520, BT474_cells |
| 126730 | T19477 | | A1426R Heart H sapiens cDNA clone A1426, | 2.04 | EB_cells, Lu_AD_H23, Lu_SC_H69 |
| 125244 | W86466 | Hs.132756 | ESTs; Weakly similar to KIAA0591 protein | 2.04 | EB_cells, Lu_AD_H23, Lu_LC_H460 |
| 134762 | M91036 | Hs.242985 | hemoglobin; gamma G | 2.04 | MB231_cells, Lu_AD_358, HT29_cells |
| 119564 | W38206 | | Accession not listed in Genbank | 2.04 | BT474_cells, HT29_cells, Lu_AD_H23 |
| 132523 | AB002332 | Hs.50722 | clock (mouse) homolog | 2.04 | PC3_cells, OVCAR_cells, PRSC_log |
| 127758 | AI337031 | Hs.180195 | ESTs | 2.04 | 293T_cells, MB-MDA-435s, A549_cells |
| 126471 | AA158755 | Hs.175652 | ESTs; Weakly similar to !!!! ALU SUBFAMI | 2.04 | EB_cells, Lu_AR_358, Lu_LC_H460 |
| 110911 | N45120 | Hs.22305 | ESTs | 2.03 | Lu_AD_H23, RPWE_2, Lu_LC_H460 |
| 122317 | AA442742 | Hs.8693 | ESTs; Weakly similar to !!!! ALU SUSFAMI | 2.03 | EB_cells, Fibroblasts 2, Lu_SC_H345 |
| 100253 | D38024 | Hs.247951 | Humn facioscapulohumeral muscular dystro | 2.03 | Lu_AD_H23, Lu_AD_358, Lu_SQ_H520 |
| 120431 | AA236854 | Hs.247323 | H sapiens mRNA for G4 protein (G4 gene; | 2.03 | Lu_SQ_H69, EB_cells, Lu_SC_H345 |
| 122449 | AA447638 | Hs.104977 | ESTs | 2.03 | Lu_SC_H345, Lu_SC_H345, Lu_SQ_H520 |
| 100961 | J00148 | | Accession not listed in Genbank | 2.03 | HT29_cells, BT474_cells, HMEC |
| 130908 | W86389 | Hs.21122 | ESTs; Moderately similar to KIAA0438 [H. | 2.03 | 293T_cells, Lu_SC_H345, OVCAR_cells |
| 102643 | U67649 | | Human bota-galactoside alpha2,6-sialyltr | 2.03 | HT29_cells, 293T_cells, Lu_SC_H345 |
| 127932 | AA398510 | Hs.133148 | ESTs | 2.03 | EB_cells, Lu_SC_H345, Lu_SC_H69 |
| 109207 | AA190906 | Hs.204692 | ESTs | 2.03 | Lu_SQ_H520, Lu_SQ_H345, Lu_SC_H69 |
| 102598 | U62962 | Hs.106673 | eukatyotic translation initiation factor | 2.03 | EB_cells, DU145_cells, MCF7 |
| 124470 | N51702 | Hs.101392 | ESTs | 2.03 | HT29_cells, Fibroblasts 2, HMEC (total RNA) |
| 104961 | AA076672 | Hs.33905 | ESTs | 2.03 | Caco2, LNCaP_cells, EB_cells |
| 124164 | H30667 | Hs.7535 | ESTs; Highly similar to COBW-like placen | 2.03 | CALU6_cells, CALU6_cells, A549_cells |
| 126468 | AA242853 | Hs.237856 | ESTs; Moderately similar to cAMP inducib | 2.03 | MB231_cells, BT474_cells, Fibroblasts 2 |
| 129683 | W05348 | Hs.158196 | DKFZP434B103 protein | 2.03 | HT29_cells, MB-MDA-435s, Lu_AD_H23 |
| 105350 | AA235737 | Hs.186571 | ATPase; Na+/K+ transporting; alpha 3 pol | 2.03 | MB-MDA-453, Lu_SQ_H520, Lu_AD_358 |
| 129794 | AA447772 | Hs.14520 | eukaryotic translation initiation factor | 2.03 | EB_cells, Lu_AD_358, Lu_AD_H23 |
| 115664 | AA405974 | Hs.54673 | tumor necrosis factor (ligand) superfami | 2.03 | Lu_AD_358, HT29_cells, HT29_cells |
| 119096 | R41672 | Hs.91471 | ATPase type IV; phospholipid transportin | 2.03 | HT29_cells, MB231_cells, BT474_cells |
| 133866 | L36151 | Hs.171625 | phosphatidylinositol 4-kinase; catalytic | 2.03 | 293T_cells, DU145_cells, LNCaP_cells |
| 132055 | N69440 | Hs.38132 | ESTs | 2.03 | Lu_SC_H345, MB-MDA-453, MB-MDA-435s |
| 125691 | AI034361 | Hs.135150 | lung type-I cell membrane-associated gly | 2.03 | Lu_SC_H345, LNCaP_cells, DU145_cells |
| 121376 | AA405699 | Hs.166232 | ESTs; Moderately similar to SODIUM-AND | 2.03 | LNCaP_cells, HT29_cells, RPWE_2 |
| | | | TRANSPORTER 2 [H.sapiens] | | |
| 105289 | AA233178 | Hs.103000 | KIAA0831 protein | 2.02 | PC3_cells, Lu_AD_H23, MB231_cells |
| 100967 | J02621 | Hs.251064 | high-mobility group (nonhistone chromoso | 2.02 | MCF7, DU145_cells, OVCAR_cells |
| 124430 | N38913 | Hs.221575 | ESTs | 2.02 | MB-MDA-435s, Fibroblasts 2, EB_cells |
| 128322 | AI306331 | Hs.133296 | ESTs | 2.02 | HT29_cells, MB-MDA-435s, Lu_SC_H345 |
| 131077 | X91809 | Hs.22698 | G alpha interacting protein | 2.02 | Lu_AD_H23, RPWE_2, MCF7 |
| 108033 | AA040923 | Hs.92200 | KIAA0480 gene product | 2.02 | MCF7, Fibroblasts 2, DU145_cells |
| 107550 | AA001045 | Hs.46783 | ESTs | 2.02 | DU145_cells, PC3_cells, OVCAR_cells |
| 109475 | AA233159 | Hs.87131 | ESTs | 2.02 | HT29_cells, MB-MDA-435s, Lu_SC_H69 |
| 111400 | R00144 | Hs.189771 | ESTs | 2.02 | HT29_cells, Fibroblasts 2, HMEC |
| 117516 | N32495 | Hs.151560 | ESTs | 2.02 | HT29_cells, HMEC (total RNA), Fibroblasts 2 |
| 120506 | AA257955 | Hs.173705 | ESTs; Weakly similar to !!!! ALU CLASS C | 2.02 | MCF7, Fibroblasts 2, LNCaP_cells |
| 130850 | N39308 | Hs.20237 | DKFZPS66C134 protein | 2.02 | EB_cells, Lu_AD_H23, Lu_LC_H460 |
| 123118 | AA486571 | Hs.105696 | ESTs; Moderately similarto !!!! ALU SUB | 2.02 | CALU6_cells, 293T_cells, PRSC_log |
| 111285 | N71704 | Hs.4310 | eukaryotic translation initiation factor | 2.02 | 293T_cells, PC3_cells, EB_cells |
| 119106 | R42362 | Hs.91785 | ESTs | 2.02 | CALU6_cells, MB-MDA-453, PC3_cells |
| 111370 | N92915 | Hs.94031 | brefeldin A-inhibited guanine nucleotide | 2.02 | EB_cells, OVCAR_cells, LNCaP_cells |
| 125013 | T67261 | Hs.154431 | ESTs; Weakly similarto neuronal thread | 2.02 | Lu_SC_H345, Lu_SC_H69, PRSC_con |
| 129762 | AA460273 | Hs.12372 | KIAA0517 protein | 2.02 | EB_cells, MB-MDA-435s, OVCAR_cells |
| 120704 | AA291970 | Hs.107054 | KIAA0821 protein | 2.01 | Lu_SC_H69, EB_cells, MB-MDA-453 |
| 105355 | AA235985 | Hs.26938 | Human DNA seq from clone 126A5 on chromo | 2.01 | Lu_AD_H23, Lu_LC_H460, Lu_SQ_H520 |
| | | | genes (one with DnaJ domains); the gene | | |
| | | | family member HKR3. Contains ESTs; STSs; | | |
| 125952 | AA017723 | | small inducible cytokine A5 (RANTES) | 2.01 | LNCaP_cells, DU145_cells, MB231_cells |
| 103478 | Y07755 | Hs.38991 | S100 calcium-binding protein A2 | 2.01 | HMEC (total RNA), HMEC, RPWE_2 |
| 133544 | T33873 | Hs.74624 | protein tyrosine phosphatase; receptor t | 2.01 | Lu_SC_H345, BT474_cells, HT29_cells |
| 112746 | R93237 | | yq11e10.s1 Soares fetal liver spleen 1NF | 2.01 | PC3_cells, LNCaP_cells, OVCAR_cells |
| | | | IMAGE: 196650 3′, mRNA seq. | | |
| 118513 | N67504 | Hs.40061 | ESTs | 2.01 | Lu_SC_H345, Lu_SC_H69, PRSC_con |
| 123423 | AA598484 | Hs.238476 | EST | 2.01 | EB_cells, Lu_AD_H23, Lu_SC_H345 |
| 127854 | AA769520 | | ESTs; Weakly similar to REGULATOR OF MIT | 2.01 | HS578T_cells, CALU6_cells, Lu_SQ_H520 |
| 111843 | R36969 | Hs.18888 | ESTs | 2.01 | Lu_AD_H23, Lu_AD_358, Lu_SQ_H520 |
| 100221 | D28383 | | Human mRNA for ATP synthase B chain, 5′U | 2.01 | EB_cells, Lu_AD_H23, LNCaP_cells |
| 129968 | AA452237 | Hs.194443 | ESTs; Weakly similar to BC37295_2 [H.sap | 2.01 | Lu_SC_H345, Lu_SC_H69, DU145_calls |
| 106798 | AA478968 | Hs.20558 | ESTs | 2.01 | EB_cells, Lu_AD_H23, Lu_LC_H460 |
| 114636 | AA085374 | | zn13dS.s1 Stratagene hNT neuron (#937233 | 2.01 | EB_cells, CALU6,_cells, OVCAR_cells |
| | | | gb:L8441 CYTOCHROME C OXIDASE | POLYPEPTI | |
| 125348 | H21585 | Hs.191277 | ESTs; Moderately similar to ATP binding | 2.01 | EB_cells, HS578T_cells, PC3_cells |
| 130620 | AA233245 | Hs.16773 | ESTs | 2.01 | EB_cells, DU145_cells, 293T_cells |
| 106471 | AA450118 | Hs.25722 | ESTs; Weakly similar to ZINC FINGER PROT | 2.01 | OVCAR_cells, LNCaP_cells, EB_cells |
| 134175 | T33128 | Hs.7966 | ESTs | 2 | Lu_SC_H345, Fibroblasts 2, Lu_AD_H23 |
| 117291 | N22289 | | yw36g08.s1 Morton Fetal Cochlea H sapien | 2 | MB-MDA-453, OVCAR_cells, CALU6_cells |
| 134199 | U47635 | Hs.79877 | myotubularin related protein 6 | 2 | EB_cells, PC3_cells, LNCaP_cells |
| 128758 | AA129545 | Hs.181165 | eukaryotic translation elongation factor | 2 | Lu_SC_H69, EB_cells, Lu_SC_H345 |
| 112005 | R42569 | Hs.22444 | ESTs | 2 | Lu_AD_H23, PRSC_log, Lu_AD_358 |
| 122521 | AA449433 | Hs.149227 | ESTs; Weakly similar to PROLINE-RICH PRO | 2 | HT29_cells, RPWE_2, MB231_cells |
| 130368 | X84373 | Hs.155017 | nuclear receptor interacting protein 1 | 2 | DU145_cells, PC3_cells, MCF7 |
| 114067 | Z38153 | Hs.26921 | ESTs | 2 | 293T_cells, MB-MDA-435s, HT29_cells |
| 107136 | AA620795 | Hs.8207 | ESTs | 2 | LNCaP_cells, PC3_cells, EB_cells |

TABLE 3

| | |
|---|---|
| Pkey: | Unique Eas probeset identifier number |
| ExAcon: | Exemplar Accession number, Genbank accession number |
| UnigeneID: | Unigene number |
| Unigene Title: | Unigene gene title |

| | | | | Ratio | |
|---|---|---|---|---|---|
| Pkey | Ex Accn | UG_ID | Complete_Title | BS/Met | Top 3 expressing cell lines |
| 302347 | AF039400 | Hs.194059 | chloride channel; calcium activated; fam | 19.71 | EB, NCI-H520, NCI-H23 |
| 316304 | AI936587 | Hs.221599 | ESTs | 14.49 | PRSC_con, RPWE-2, OVCA-R |
| 339196 | | | CH22_FF113D11.GENSCAN.3-1 | 10.37 | NCI-H69, PRSC_con, NCI-H345 |
| 336171 | | | CH22_FGENES.708_3 | 9.45 | NCI-H69, NCI-H460, NCI-H23 |
| 338895 | | | CH22_DJ32I10.GENSCAN.9-2 | 9.31 | PC3, BT474, OVCA-R |
| 333625 | | | CH22_FGENES.223_2 | 8.96 | NCI-H69, PRSC_con, NCI-H345 |
| 333730 | | | CH22_FGENES.258_1 | 8.82 | NCI-H69, BT474, MB-MDA-231 |
| 320244 | AA296922 | Hs.129778 | gastrointestinal peptide | 8.22 | BT474, CALU6, DU145 |
| 333643 | | | CH22_FGENES.232_2 | 7.66 | MCF7, NCI-H69, LnCap |
| 333423 | | | CH22_FGENES.147_3 | 7.57 | HT29, MB-MDA-231, EB |
| 302332 | AI833168 | Hs.184507 | H sapiens Chromosome 16 BAC clone CIT987 | 7.55 | MB-MDA-231, HT29, MB-MDA-453 |
| 333588 | | | CH22_FGENES.206_2 | 7.46 | HT29, OVCA-R, BT474 |
| 322033 | AL137507 | | EST cluster (not in UniGene) | 7.35 | PRSC_con, PRSC_log, NCI-H345 |
| 308601 | AI719930 | | EST singleton (not in UniGene) with exon | 6.83 | PC3, DU145, DU145 |
| 339044 | | | CH22_DA59H18.GENSCAN.27-5 | 6.46 | NCI-H69, NCI-H345, PRSC_log |
| 314516 | AA371513 | Hs.231748 | ESTs | 6.41 | EB, OVCA-R, Caco2 |
| 327805 | | | CH.05_hs gi\|5867968 | 6.28 | NCI-H69, NCI-H345, PRSC_con |
| 334239 | | | CH22_FGENES.364_2 | 6.09 | NCI-H520, MB-MDA-435s, MB-MDA-453 |
| 332958 | | | CH22_FGENES.48_15 | 6.04 | NCI-H69, PRSC_con, PRSC_log |
| 313386 | W85772 | Hs.173924 | ESTs | 5.88 | MB-MDA-231, OVCA-R, BT474 |
| 314350 | AL037927 | Hs.190675 | ESTs; Moderately similar to !!!! ALU SUB | 5.84 | OVCA-R, CALU6, EB |
| 337170 | | | CH22_FGENES.564-1 | 5.67 | LnCap, CALU6, NCI-H69 |
| 337503 | | | CH22_FGENES.803-1 | 5.66 | NCI-H345, PRSC_con, RPWE-2 |
| 337562 | | | CH22_C65E1.GENSCAN.1-2 | 5.53 | HT29, MB-MDA-453, BT474 |
| 337219 | | | CH22_FGENES.614-3 | 5.45 | NCI-H69, NCI-H345, PRSC_log |
| 311331 | AI679622 | Hs.32225 | immunoglobulin alpha 1 | 5.43 | NCI-H69, NCI-H23, NCI-H345 |
| 314251 | AA713589 | | EST cluster (not in UniGene) | 5.41 | PC3, EB, LnCap |
| 336246 | | | CH22_FGENES.746_5 | 5.34 | NCI-H69, NCI-H345, PRSC_log |
| 335009 | | | CH22_FGENES.472_13 | 5.31 | ES, EB, NCI-H69 |
| 339365 | | | CH22_BA354I12.GENSCAN.34-1 | 5.25 | PRSC_con, NCI-H69, PRSC_log |
| 336088 | | | CH22_FGENES.688_17 | 5.21 | PRSC_con, Caco2, PRSC_log |
| 334966 | | | CH22_FGENES.465_36 | 5.16 | DU145, BT474, MB-MDA-231 |
| 334666 | | | CH22_FGENES.418_18 | 5.15 | NCI-H69, NCI-H345, PRSC_log |
| 316830 | AW182106 | Hs.127821 | ESTs | 5.12 | NCI-H345, PRSC_con, PRSC_log |
| 339413 | | | CH22_DJ579N16.GENSCAN.5-8 | 5.06 | NCI-H69, NCI-H345, PRSC_log |
| 337951 | | | CH22_EM:AC005500.GENSCAN.94-1 | 5.01 | NCI-H345, NCI-H69, PRSC_con |
| 330153 | | | CH21_p2 gi\|4325335 | 5 | PRSC_con, PRSC_log, NCI-H69 |
| 333987 | | | CH22_FGENES.310_11 | 4.96 | MB-MDA-231, MB-MDA-453, MB-MDA-453 |
| 334304 | | | CH22_FGENES.373_7 | 4.96 | OVCA-R, CALU6, NCI-H23 |
| 338990 | | | CH22.DA59H18.GENSCAN.6-6 | 4.95 | PRSC_log, PRSC_con, NCI-H69 |
| 333152 | | | CH22_FGENES.89_1 | 4.89 | MB-MDA-435s, OVCA-R, A549 |
| 327049 | | | CH.21_hs gi\|6531965 | 4.87 | PRSC_con, NCI-H345, PRSC_log |
| 337225 | | | CH22_FGENES.626-3 | 4.83 | DU145, CALU6, EB |
| 333496 | | | CH22_FGENES.168_6 | 4.81 | NCI-H69, NCI-H345, PRSC_con |
| 334451 | | | CH22_FGENES.387_11 | 4.79 | RPWE-2, PRSC_con, NCI-H69 |
| 333594 | | | CH22_FGENES.210_3 | 4.78 | OVCA-R, PC3, HT29 |
| 333635 | | | CH22_FGENES.228_2 | 4.78 | NCI-H69, PRSC_log, PRSC_con |
| 336796 | | | CH22.FGENES.176-6 | 4.73 | NCI-H69, NCI-H345, PRSC_log |
| 333313 | | | CH22_FGENES.138_5 | 4.72 | NCI-H69, NCI-H345, PRSC_log |
| 336833 | | | CH22_FGENES.242-2 | 4.7 | NCI-H345, NCI-H69, PRSC_con |
| 336090 | | | CH22_FGENES.689_2 | 4.7 | NCI-H69, PRSC_con, PRSC_log |
| 336645 | | | CH22_FGENES.26-1 | 4.63 | HT29, OVCA-R, DU145 |
| 334565 | | | CH22_FGENES.405_5 | 4.62 | NCI-H345, PRSC_log, RPWE-2 |
| 333242 | | | CH22_FGENES.111_6 | 4.56 | NCI-H345, PRSC_log, PRSC_con |
| 326304 | | | CH.17_hs gi\|5867277 | 4.48 | OVCA-R, EB, DU145 |
| 337445 | | | CH22_FGENES.769-4 | 4.47 | RPWE-2, NCI-H69, PRSC_log |
| 327413 | | | CH.02_hs gi\|5867750 | 4.46 | NCI-H69, PRSC_log, NCI-H345 |
| 327990 | | | CH.06_hs gi\|5868218 | 4.44 | PRSC_con, PRSC_log, RPWE-2 |
| 325038 | H38304 | Hs.21782 | ESTs | 4.43 | PRSC_con, MB-MDA-231, HT29 |
| 314923 | AI732489 | Hs.136370 | ESTs | 4.4 | HT29, MB-MDA-231, NCI-358 |
| 328859 | | | CH.07_hs gi\|6381928 | 4.4 | OVCA-R, BT474, A549 |
| 334476 | | | CH22_FGENES.394_7 | 4.38 | OVCA-R, PC3, EB |
| 336092 | | | CH22_FGENES.689_6 | 4.35 | PRSC_con, Caco2, PRSC_log |
| 333965 | | | CH22_FGENES.305_3 | 4.35 | NCI-H69, NCI-H345, PRSC_log |
| 336402 | | | CH22.FGENES.823_17 | 4.34 | RPWE-2, HT29, OVCA-R |
| 337947 | | | CH22_EM:AC005500.GENSCAN.90-5 | 4.33 | OVCA-R, DU145, PC3 |
| 337504 | | | CH22_FGENES.803-2 | 4.33 | NCI-H345, PRSC_con, PRSC_log |
| 336813 | | | CH22_FGENES.213-6 | 4.33 | DU145, HT29, OVCA-R |
| 338069 | | | CH22_EM:AC005500.GENSCAN.166-14 | 4.33 | NCI-H69, PRSC_con, NCI-H345 |
| 318538 | N28625 | Hs.74034 | caveolin 1; caveolae protein; 22 kD | 4.31 | PC3, A549, BT474 |
| 333631 | | | CH22_FGENES.227_2 | 4.3 | OVCA-R, PRSC_con, LnCap |
| 302646 | M14268 | | EST | 4.27 | PRSC_con, PRSC_log, RPWE-2 |
| 336049 | | | CH22.FGENES.681_2 | 4.26 | HT29, DU145, DU145 |
| 335667 | | | CH22_FGENES.590_18 | 4.25 | NCI-H520, Caco2, MB-MDA-453 |
| 320352 | Y13323 | Hs.145296 | disintegrin protease | 4.25 | MB-MDA-231, DU145, BT474 |
| 304480 | AA430373 | | EST singleton (not in UniGene) with exon | 4.22 | NCI-358, NCI-H460, NCI-H23 |
| 327273 | | | CH.01_hs gi\|5867466 | 4.22 | NCI-H69, NCI-H345, PRSC_con |
| 334540 | | | CH22FGENES.403_5 | 4.17 | NCI-H69, NCI-H345, PRSC_log |
| 334719 | | | CH22_FGENES.421_30 | 4.17 | NCI-H69, NCI-H345, RPWE-2 |
| 327827 | | | CH.05_hs gi\|5867968 | 4.17 | OVCA-R, NCI-H69, CALU6 |
| 333599 | | | CH22_FGENES.212_2 | 4.17 | PRSC_log, NCI-H69, PRSC_con |
| 329638 | | | CH.12_p2 gi\|3779004 | 4.16 | DU145, MB-MDA-231, HT29 |
| 307556 | AI281651 | | EST singleton (not in UniGene) with exon | 4.16 | BT474, HT29, CALU6 |
| 336836 | | | CH22_FGENES.247-11 | 4.15 | PRSC_con, NCI-H345, NCI-H69 |
| 323187 | AL121180 | Hs.240038 | ESTs | 4.14 | NCI-H345, MB-MDA-435s, RPWE-2 |
| 336397 | | | CH22_FGENES.823.12 | 4.13 | NCI-H345, PRSC_con, RPWE-2 |
| 325007 | AA736429 | | EST cluster (not in UniGene) | 4.13 | NCI-H69, PRSC_con, NCI-H345 |
| 300199 | AI304386 | Hs.150836 | ESTs | 4.11 | NCI-H345, PRSC_con, PRSC_log |
| 335832 | | | CH22_FGENES.620_6 | 4.08 | NCI-H69, NCI-H345, PRSC_log |
| 312778 | AI631655 | Hs.197919 | ESTs | 4.07 | NCI-358, NCI-H23, PRSC_con |
| 323154 | AA765301 | Hs.151858 | ESTs | 4.06 | NCI-H23, A549, HT29 |
| 315871 | AW135312 | Hs.117237 | ESTs | 4.05 | MB-MDA-231, EB, MCF7 |
| 337452 | | | CH22_FGENES.775-1 | 4.02 | PRSC_con, PRSC_log, NCI-H345 |
| 335265 | | | CH22_FGENES.521_1 | 4.01 | NCI-H69, MCF7, RPWE-2 |
| 335200 | | | CH22_FGENES.508_9 | 4.01 | NCI-H69, PRSC_log, PRSC_con |
| 336917 | | | CH22_FGENES.346-4 | 3.99 | PRSC_con, NCI-H345, PRSC_log |
| 336584 | | | CH22_FGENES.847_1 | 3.98 | PRSC_log, PRSC_con, RPWE-2 |
| 333382 | | | CH22_FGENES.143_21 | 3.97 | EB, A549, HT29 |
| 329436 | | | CH.Y_hs gi\|5868883 | 3.97 | BT474, PC3, HT29 |
| 336929 | | | CH22_FGENES.349-3 | 3.94 | NCI-H69, NCI-H345, PRSC_log |
| 337238 | | | CH22.FGENES.641-3 | 3.92 | NCI-H69, NCI-H345, PRSC_log |
| 333875 | | | CH22.FGENES.291_11 | 3.92 | PRSC_con, RPWE-2, PRSC_log |
| 337069 | | | CH22_FGENES.448.2 | 3.9 | NCI-H69, LnCap, RPWE-2 |
| 332491 | M24470 | Hs.1435 | guanosine monophosphate reductase | 3.86 | OVCA-R, MB-MDA-435s, CALU6 |
| 304623 | AA521331 | | EST singleton (not in UniGene) with exon | 3.86 | OVCA-R, DU145, PC3 |
| 335348 | | | CH22_FGENES.537_4 | 3.85 | HT29, MB-MDA-231, PC3 |
| 334568 | | | CH22_FGENES.405_9 | 3.85 | NCI-H69, NCI-H345, PRSC_log |
| 336924 | | | CH22.FGENES.347-9 | 3.84 | NCI-H345, PRSC_log, RPWE-2 |
| 301654 | H81795 | | EST | 3.84 | NCI-H520, LnCap, NCI-358 |
| 334677 | | | CH22_FGENES.418_30 | 3.83 | PRSC_con, NCI-H345, NCI-H69 |
| 326688 | | | CH.20.hs gi\|5867582 | 3.83 | NCI-H345, PRSC_con, PRSC_log |
| 327790 | | | CH.05_hs gi\|5867977 | 3.8 | PRSC_con, PRSC_log, NCI-H345 |
| 334591 | | | CH22_FGENES.408_1 | 3.8 | NCI-H69, PRSC_log, NCI-H345 |
| 337974 | | | CH22_EM:AC005500.GENSCAN.106-3 | 3.78 | PRSC_log, PRSC_con, NCI-H345 |
| 311274 | AW293128 | Hs.197101 | ESTs | 3.78 | NCI-H345, PRSC_con, RPWE-2 |
| 326668 | | | CH.20_hs gi\|6552455 | 3.78 | NCI-H345, NCI-H69, PRSC_log |
| 304195 | N35382 | | EST singleton (not in UniGene) with exon | 3.77 | NCI-H69, RPWE-2, PRSC_con |
| 336294 | | | CH22_FGENES.786_4 | 3.77 | PRSC_con, PRSC_log, NCI-H69 |
| 311613 | AL046311 | Hs.252443 | ESTs; Weakly similar to !!!! ALU SUBFAMI | 3.76 | HT29, BT474, MB-MDA-231 |
| 338123 | | | CH22_EM:AC005500.GENSCAN.195-5 | 3.75 | MB-MDA-231, HT29, BT474 |
| 318230 | AA558125 | | EST cluster (not in UniGene) | 3.74 | RPWE-2, PRSC_con, NCI-H345 |
| 303985 | AW514501 | Hs.156110 | Immunoglobulin kappa variable 1D-8 | 3.73 | MB-MDA-231, BT474, PRSC_con |
| 336502 | | | CH22_FGENES.833_8 | 3.72 | NCI-H345, RPWE-2, PRSC_con |
| 334063 | | | CH22_FGENES.327_17 | 3.71 | NCI-H69, NCI-H345, PRSC_con |
| 333600 | | | CH22_FGENES.213_2 | 3.7 | NCI-H69, OVCA-R, PC3 |
| 339424 | | | CH22_DJ579N16.GENSCAN.14-3 | 3.69 | NCI-H69, NCI-H345, PRSC_con |
| 336862 | | | CH22_FGENES.297-2 | 3.67 | NCI-H345, PRSC_con, PRSC_log |
| 334823 | | | CH22.FGENES.437_5 | 3.67 | RPWE-2, PRSC_log, PRSC_con |
| 329940 | | | CH.16_p2 gi\|6165199 | 3.66 | CALU6, EB, MCF7 |
| 300275 | A1632123 | Hs.231521 | ESTs | 3.66 | PRSC_con, NCI-H69, RPWE-2 |
| 328820 | | | CH.07_hs gi\|5868330 | 3.66 | NCI-H69, NCI-H345, PRSC_con |
| 332398 | AA446446 | Hs.104788 | H sapiens clone 24554 unknown mRNA | 3.66 | PRSC_con, PRSC_log, NCI-H345 |
| 325791 | | | CH.14_hs gi\|6682476 | 3.65 | NCI-H345, BT474, LnCap |
| 300672 | R14469 | Hs.258573 | ESTs | 3.65 | MCF7, MB-MDA-453, MB-MDA-435s |
| 338344 | | | CH22_EM:AC005500.GENSCAN.312-8 | 3.65 | NCI-H345, PRSC_log, PRSC_con |
| 333257 | | | CH22_FGENES.118_5 | 3.65 | DU145, EB, OVCA-R |
| 332140 | AA620724 | Hs.112890 | ESTs | 3.65 | MB-MDA-453, DU145, MCF7 |
| 337489 | | | CH22_FGENES.799-2 | 3.63 | NCI-H345, NCI-H69, PRSC_log |
| 305167 | AA663080 | | EST singleton (not in UniGene) with exon | 3.63 | OVCA-R, MB-MDA-231, MB-MDA-435s |
| 336200 | | | CH22_FGENES.719_4 | 3.61 | NCI-H69, PRSC_log, NCI-H345 |
| 339208 | | | CH22_FF113D11.GENSCAN,6-3 | 3.59 | PRSC_con, NCI-H69, PRSC_log |
| 320090 | AB002058 | Hs.113275 | purinergic receptor P2X-Iike 1; orphan r | 3.58 | OVCA-R, LnCap, NCI-H69 |
| 335999 | | | CH22_FGENES.657_1 | 3.57 | NCI-H345, NCI-H69, PRSC_con |
| 332909 | | | CH22_FGENES.36_13 | 3.57 | NCI-H345, PRSC_con, PRSC_log |
| 306531 | AA991423 | | EST singleton (not in UniGene) with exon | 3.56 | BT474, MB-MDA-453, MB-MDA-435s |
| 333261 | | | CH22_FGENES.119_1 | 3.55 | HT29, CALU6, MB-MDA-231 |
| 303883 | AA176396 | Hs.169624 | ESTs | 3.54 | NCI-H69, NCI-H345, RPWE-2 |
| 335831 | | | CH22_FGENES.620_5 | 3.53 | MCF7, BT474, OVCA-R |
| 333983 | | | CH22_FGENES.310_7 | 3.52 | NCI-H345, PRSC_con, PRSC_log |
| 333623 | | | CH22_FGENES.222_2 | 3.51 | NCI-H69, PRSC_con, PRSC_log |
| 333997 | | | CH22_FGENES.310_22 | 3.5 | NCI-H345, PRSC_con, PRSC_log |
| 325623 | | | CH.14_hs gi\|5867000 | 3.5 | CALU6, HT29, BT474 |
| 309151 | AI935829 | Hs.140 | immunoglobulin gamma 3 (Gm marker) | 3.49 | EB, MCF7, MB-MDA-453 |
| 305080 | AA641485 | | EST singleton (not in UniGene) with exon | 3.49 | NCI-H23, NCI-H460, NCI-358 |
| 339268 | | | CH22_BA354I12.GENSCAN.10-6 | 3.47 | NCI-H69, NCI-H345, PRSC_con |
| 310048 | AI198352 | Hs.105077 | ESTs | 3.47 | Caco2, PRSC_con, NCI-H69 |
| 314758 | AA521458 | Hs.192738 | ESTs | 3.46 | NCI-H23, NCI-H23, NCI-H520 |
| 334664 | | | CH22_FGENES.418_15 | 3.45 | NCI-H69, PRSC_log, PRSC_con |
| 334661 | | | CH22_FGENES.418_9 | 3.45 | NCI-H69, PRSC_con, PRSC_log |
| 330984 | H38678 | Hs.32766 | H sapiens clone 24803 mRNA seq | 3.44 | OVCA-R, MCF7, PC3 |
| 333464 | | | CH22_FGENES.160_1 | 3.44 | NCI-H69, MB-MDA-231, MCF7 |
| 333580 | | | CH22_FGENES.199_2 | 3.42 | PRSC_con, NCI-H69, PRSC_log |
| 313356 | AI266254 | Hs.132929 | ESTs | 3.42 | RPWE-2, PRSC_con, NCI-H345 |
| 334518 | | | CH22_FGENES.400_1 | 3.41 | PRSC_log, PRSC_con, RPWE-2 |
| 333627 | | | CH22_FGENES.225_2 | 3.4 | HT29, BT474, BT474 |
| 309641 | AW194230 | Hs.253100 | EST | 3.4 | HT29, MB-MDA-453, MCF7 |
| 338221 | | | CH22_EM:AC005500.GENSCAN.246-10 | 3.4 | NCI-H69, PRSC_log, NCI-H345 |
| 312993 | AI392673 | Hs.125230 | ESTs | 3.4 | PRSC_log, NCI-H345, NCI-H345 |
| 318336 | AI971806 | Hs.164158 | ESTs | 3.38 | OVCA-R, ES, CALU6 |
| 326218 | | | CH.17_hs gi\|5867226 | 3.38 | NCI-H460, NCI-H69, NCI-H345 |
| 336231 | | | CH22_FGENES.736_3 | 3.38 | NCI-H69, NCI-H345, PRSC_log |
| 307912 | AI382224 | | EST singleton (not in UniGene) with exon | 3.37 | NCI-H345, PRSC_con, RPWE-2 |
| 336161 | | | CH22FGENES.707_6 | 3.37 | NCI-H69, NCI-H345, RPWE-2 |
| 300875 | AW134756 | Hs.192477 | ESTs | 3.37 | RPWE-2, PRSC_log, PRSC_con |
| 336593 | | | CH22_FGENES.135_1 | 3.37 | PRSC_con, NCI-H69, RPWE-2 |
| 310696 | AI431620 | Hs.160875 | ESTs | 3.36 | HT29, OVCA-R, BT474 |
| 304745 | AA577771 | | EST singleton (not in UniGene) with exon | 3.36 | NCI-H345, RPWE-2, PRSC_con |
| 308911 | AI860287 | Hs.156110 | Immunoglobulin kappa variable 1D-8 | 3.36 | EB, DU145, CALU6 |
| 336347 | | | CH22.FGENES.815_3 | 3.36 | NCI-H69, PRSC_log, PRSC_con |
| 334906 | | | CH22_FGENES.452_21 | 3.33 | Caco2, CALU6, MB-MDA-453 |
| 334548 | | | CH22_FGENES.403_13 | 3.33 | NCI-H345, PRSC_con, NCI-H69 |
| 336695 | | | CH22_FGENES.48-4 | 3.32 | NCI-H69, PRSC_log, PRSC_con |
| 316684 | AA807187 | Hs.220783 | ESTs; Weakly similar to WNT-1 PROTO-ONCO | 3.31 | DU145, ES, MB-MDA-231 |
| 315901 | AI521558 | Hs.179718 | v-myb avian myeloblastosis viral oncogen | 3.3 | Caco2, LnCap, NCI-H69 |
| 320115 | T93574 | | EST cluster (not in UniGene) | 3.3 | DU145, HT29, CALU6 |
| 307847 | AI363993 | Hs.157273 | EST | 3.3 | NCI-H345, PRSC_con, PRSC_log |
| 327899 | | | CH.06_hs gi\|5868156 | 3.28 | BT474, MB-MDA-231, A549 |
| 304612 | AA514207 | | EST singleton (not in UniGene) with exon | 3.28 | DU145, CALU6, LnCap |
| 330021 | | | CH.16_p2 gi\|6671889 | 3.27 | A549, HT29, EB |
| 338132 | | | CH22_EM:AC005500.GENSCAN.200-2 | 3.27 | MB-MDA-231, CALU6, EB |
| 323690 | AA317497 | Hs.188897 | ESTs | 3.27 | RPWE-2, NCI-H345, MCF7 |
| 327362 | | | CH.01_hs gi\|6552412 | 3.26 | NCI-H69, RPWE-2, PRSC_log |
| 333488 | | | CH22.FGENES.167_3 | 3.26 | NCI-H69, NCI-H345, PRSC_log |
| 334106 | | | CH22_FGENES.330_5 | 3.26 | NCI-H69, PRSC_con, PRSC_log |
| 306990 | AI129298 | Hs.146491 | EST; Weakly similar to FERRITIN HEAVY CH | 3.26 | NCI-H345, PRSC_log, PRSC_con |
| 328420 | | | CH.07_hs gi\|5868411 | 3.26 | NCI-H69, NCI-H345, PRSC_log |
| 336214 | | | CH22_FGENES.722_8 | 3.26 | MCF7, ES, OVCA-R |
| 330565 | U51095 | Hs.1545 | caudal type homeo box transcription fact | 3.25 | EB, DU145, HT29 |
| 333879 | | | CH22_FGENES.291_15 | 3.25 | PRSC_con, PRSC_log, NCI-H69 |
| 300145 | AI240850 | Hs.232016 | ESTs | 3.25 | NCI-H345, PRSC_con, PRSC_log |
| 327581 | | | CH.03_hs gi\|5867825 | 3.25 | EB, DU145, MB-MDA-453 |
| 308153 | AI500429 | Hs.1103 | transforming growth factor; beta 1 | 3.24 | MCF7, EB, EB |
| 308337 | AI608947 | | EST singleton (not in UniGene) with exon | 3.24 | PRSC_log, PRSC_con, NCI-H345 |
| 329406 | | | CH.X_hs gi\|6682547 | 3.23 | DU145, HT29, MB-MDA-231 |
| 325482 | | | CH.12_hs gi\|5866957 | 3.23 | NCI-H69, NCI-H345, PRSC_con |
| 337544 | | | CH22_FGENES.833-7 | 3.22 | NCI-H69, NCI-H345, PRSC_con |
| 337204 | | | CH22_FGENES.595-1 | 3.22 | NCI-H69, PRSC_con, PRSC_log |
| 309451 | AW105128 | Hs.246687 | EST | 3.22 | PRSC_con, RPWE-2, NCI-H345 |
| 337259 | | | CH22_FGENES.649-3 | 3.2 | PRSC_con, NCI-H345, NCI-H69 |
| 336489 | | | CH22_FGENES.831_10 | 3.2 | CALU6, MB-MDA-435s, Caco2 |
| 334804 | | | CH22_FGENES.435_4 | 3.18 | PRSC_log, PRSC_con, RPWE-2 |
| 335739 | | | CH22_FGENES.601_10 | 3.18 | NCI-H69, RPWE-2, PRSC_con |
| 306264 | AA935305 | Hs.179779 | ribosomal protein L37 | 3.17 | LnCap, NCI-H69, EB |
| 329386 | | | CH.X_hs gi\|6004484 | 3.17 | RPWE-2, NCI-H345, PRSC_log |
| 323479 | AA278246 | | EST cluster (not in UniGene) | 3.16 | PRSC_con, NCI-H345, RPWE-2 |
| 304731 | AA576085 | | EST singleton (not in UniGene) with exon | 3.16 | NCI-H69, LnCap, DU145 |
| 339419 | | | CH22_DJ579N16.GENSCAN.11-11 | 3.15 | NCI-H69, PRSC_log, RPWE-2 |
| 301202 | AI536797 | Hs.173155 | ESTs | 3.15 | LnCap, NCI-H69, Caco2 |
| 333608 | | | CH22_FGENES.216_3 | 3.15 | NCI-H345, PRSC_con, PRSC_log |
| 339193 | | | CH22_FF113D11.GENSCAN.1-5 | 3.14 | NCI-H69, NCI-H345, PRSC_con |
| 310527 | AW293404 | Hs.211986 | ESTs | 3.14 | PRSC_log, PRSC_con, RPWE-2 |
| 321146 | AA707443 | Hs.183983 | ESTs | 3.14 | PRSC_con, NCI-H69, PRSC_log |
| 333271 | | | CH22_FGENES.121_2 | 3.13 | NCI-H345, NCI-H69, RPWE-2 |
| 330280 | | | CH.05_p2 gi\|6671910 | 3.13 | NCI-H69, NCI-H345, PRSC_log |
| 309977 | AW451663 | | EST singleton (not in UniGene) with exon | 3.13 | PRSC_con, PRSC_log, RPWE-2 |
| 307588 | AI285535 | | EST singleton (not in UniGene) with exon | 3.13 | MB-MDA-231, BT474, BT474 |
| 330551 | U39840 | Hs.105440 | hepatocyte nuclear factor 3; alpha | 3.13 | MB.MDA-453, LnCap, Caco2 |
| 314404 | AW104203 | Hs.157505 | ESTs | 3.13 | DU145, EB, OVCA-R |
| 334030 | | | CH22_FGENES.320_2 | 3.13 | NCI-H69, NCI-H345, PRSC_con |
| 309108 | AI925949 | | EST singleton (not in UniGene) with exon | 3.13 | BT474, MCF7, EB |
| 317516 | AI733250 | Hs.192262 | ESTs | 3.12 | OVCA-R, EB, MB-MDA-453 |
| 304161 | H71886 | | EST singleton (not in UniGene) with exon | 3.12 | PRSC_con, NCI-H69, RPWE-2 |
| 334590 | | | CH22_FGENES.407_13 | 3.12 | NCI-H69, NCI-H345, PRSC_con |
| 333408 | | | CH22_FGENES.145_6 | 3.11 | PRSC_log, RPWE-2, PRSC_con |
| 330387 | H14624 | Hs.31386 | ESTs; Highly similar to secreted apoptos | 3.11 | DU145, OVCA-R, PC3 |
| 332567 | N23730 | Hs.25647 | v-fos FBJ murine osteosarcoma viral onco | 3.11 | EB, MB-MDA-453, MCF7 |
| 333682 | | | CH22_FGENES.247_10 | 3.1 | PRSC_con, PRSC_log, RPWE-2 |
| 323152 | AI680562 | Hs.246192 | ESTs; Weakly similar to REGULATOR OF MIT | 3.1 | PC3, MB-MDA-453, DU145 |
| 311142 | AI638441 | Hs.195649 | ESTs | 3.1 | PRSC_con, RPWE-2, PRSC_log |
| 333441 | | | CH22_FGENES.151_5 | 3.1 | RPWE-2, NCI-H345, PRSC_log |
| 326459 | | | CH.19_hs gi\|5867400 | 3.09 | EB, CALU6, PC3 |
| 313493 | AA910339 | Hs.126868 | ESTs | 3.09 | NCI-H345, PRSC_con, RPWE-2 |
| 339356 | | | CH22_SA354I12.GENSCAN.31-1 | 3.08 | NCI-H69, NCI-H345, PRSC_log |
| 333629 | | | CH22_FGENES.226_5 | 3.08 | NCI-H69, NCI-H345, PRSC_log |
| 304127 | H42981 | | EST singleton (not in UniGene) with exon | 3.07 | LnCap, PRSC_con, DU145 |
| 325691 | | | CH.14_hs gi\|5867021 | 3.07 | NCI-H345, PRSC_con, NCI-H69 |
| 333014 | | | CH22_FGENES.61_6 | 3.07 | PRSC_con, PRSC_log, NCI-H345 |
| 327379 | | | CH.02_hs gi\|5867795 | 3.07 | PRSC_con, PRSC_log, NCI-H69 |
| 337816 | | | CH22_EM:AC005500.GENSCAN.13-1 | 3.06 | NCI-H69, PRSC_con, PRSC_log |
| 337954 | | | CH22_EM:AC005500.GENSCAN.96-3 | 3.06 | PRSC_log, NCI-H69, NCI-H345 |
| 328109 | | | CH.06_hs gi\|5868020 | 3.05 | HT29, BT474, MB-MDA-231 |
| 338527 | | | CH22_EM:AC005500.GENSCAN.396-15 | 3.05 | NCI-H69, NCI-H345, PRSC_con |
| 320083 | T87761 | | EST duster (not in UniGene) | 3.05 | BT474, MS-MDA-435s, MCF7 |
| 333486 | | | CH22_FGENES.161_2 | 3.05 | NCI-H345, RPWE-2, PRSC_log |
| 334788 | | | CH22.FGENES.432_13 | 3.04 | EB, A549, CALU6 |
| 302681 | X97550 | | EST | 3.04 | OVCA-R, EB, MB-MDA-453 |
| 336238 | | | CH22_FGENES.743_3 | 3.03 | NCI-H69, PRSC_log, PRSC_con |
| 337606 | | | CH22_C20H12.GENSCAN.17-2 | 3.02 | HT29, BT474, MS-MDA-231 |
| 333545 | | | CH22_FGENES.180_1 | 3.02 | NCI-H69, NCI-H345, RPWE-2 |
| 309782 | AW275156 | Hs.156110 | Immunoglobulin kappa variable 1D-8 | 3.02 | PRSC_log, PRSC_con, RPWE-2 |
| 324277 | AA429440 | Hs.207285 | ESTs | 3.02 | BT474, MB-MDA-231, HT29 |
| 321074 | H38098 | Hs.32756 | ESTs | 3.02 | PC3, BT474, MB-MDA-231 |
| 337094 | | | CH22_FGENES.465-19 | 3.01 | PRSC_con, PRSC_log, RPWE-2 |
| 313913 | AW391342 | | EST cluster (not in UniGene) | 3 | NCI-H345, RPWE-2, PRSC_log |
| 329140 | | | CH.X_hs gi\|6017060 | 3 | EB, DU145, PC3 |
| 335331 | | | CH22.FGENES.535_4 | 3 | MS-MDA-435s, HT29, BT474 |
| 334827 | | | CH22_FGENES.437_9 | 2.99 | CALU6, EB, DU145 |
| 326029 | | | CH.17_hs gi\|5867176 | 2.99 | NCI-H345, RPWE-2, PRSC_con |
| 303100 | T09353 | | EST | 2.99 | MS-MDA-453, NCI-H345, RPWE-2 |
| 328768 | | | CH.07_hs gi\|6017031 | 2.99 | NCI-H345, PRSC_con, NCI-H69 |
| 329392 | | | CH.X_hs gi\|6478815 | 2.98 | NCI-H69, NCI-H345, PRSC_con |
| 305168 | AA663105 | | EST singleton (not in UniGene) with exon | 2.98 | LnCap, NCI-H345, MCF7 |
| 300992 | AA601213 | Hs.191798 | ESTs | 2.98 | Caco2, HT29, NCI-358 |
| 334474 | | | CH22_FGENES.394_5 | 2.98 | NCI-H69, PRSC_con, RPWE-2 |
| 322647 | AA007534 | Hs.125062 | ESTs | 2.98 | HT29, OVCA-R, A549 |
| 310620 | AI341328 | Hs.178953 | ESTs | 2.97 | PRSC_con, RPWE-2, PRSC_log |
| 328276 | | | CH.07_hs gi\|6004471 | 2.97 | NCI-H345, NCI-H69, RPWE-2 |
| 331018 | N26904 | Hs.24048 | ESTs; Weakly similar to FK506/rapamycin- | 2.96 | Caco2, NCI-H60, A549 |
| 321523 | H78472 | Hs.191325 | ESTs; Weakly similar to cDNA EST yk414c9 | 2.96 | PRSC_con, PRSC_log, NCI-H345 |
| 339280 | | | CH22_BA354I12,GENSCAN.14-12 | 2.96 | NCI-H69, PRSC_log, NCI-H345 |
| 305967 | AA886428 | | EST singleton (not in UniGene) with exon | 2.96 | NCI-H520, NCI-358, MS-MDA-453 |
| 335755 | | | CH22_FGENES.604_4 | 2.95 | EB, A549, MB-MDA-453 |
| 323907 | AL043098 | Hs.165387 | ESTs | 2.95 | PRSC_con, NCI-H345, PRSC_log |
| 330370 | | | CH.X_p2 gi\|6580495 | 2.95 | EB, DU145, MB-MDA-435s |
| 334529 | | | CH22_FGENES.402_9 | 2.94 | EB, MCF7, DU145 |
| 339256 | | | CH22_BA354I12.GENSCAN.7-11 | 2.94 | NCI-H69, NCI-H345, PRSC_con |
| 334783 | | | CH22_FGENES.432_8 | 2.94 | A549, Caco2, PC3 |
| 335266 | | | CH22_FGENES.521_2 | 2.94 | NCI-H69, PRSC_con, PRSC_con |
| 323707 | AA845957 | Hs.128385 | ESTs | 2.94 | NCI-H345, PRSC_con, PRSC_log |
| 336199 | | | CH22_FGENES.719_3 | 2.93 | NCI-H69, NCI-H345, PRSC_log |
| 338326 | | | CH22_EM:AC005500.GENSCAN.308-2 | 2.93 | NCI-H69, NCI-H345, NCI-358 |
| 333652 | | | CH22_FGENES.239_1 | 2.93 | PC3, OVCA-R, BT474 |
| 336479 | | | CH22.FGENES.829_39 | 2.92 | NCI-H69, PRSC_con, PRSC_log |
| 336086 | | | CH22_FGENES.688_15 | 2.92 | PRSC_con, Caco2, CALU6 |
| 338516 | | | CH22.EM:AC005500.GENSCAN.392-6 | 2.92 | NCI-H69, NCI-H345, PRSC_con |
| 320121 | T93657 | | EST cluster (not in UrnGene) | 2.92 | EB, BT474, HT29 |
| 305782 | AA844730 | | EST singleton (not in UniGene) with exon | 2.92 | MB-MDA-453, MCF7, DU145 |
| 339304 | | | CH22_BA354I12.GENSCAN.20-16 | 2.91 | PRSC_con, PRSC_log, NCI-H69 |
| 327472 | | | CH.02_hs gi\|5867775 | 2.91 | PRSC_log, PRSC_con, RPWE-2 |
| 311458 | AW139426 | Hs.244718 | ESTs | 2.91 | PRSC_con, PRSC_log, RPWE-2 |
| 338431 | | | CH22_EM:AC005500.GENSCAN.351-4 | 2.9 | BT474, MCF7, MB-MDA-453 |
| 339230 | | | CH22_BA354I12.GENSCAN.1-6 | 2.89 | NCI-H69, NCI-H345, PRSC_log |
| 320588 | NM_00365 | | EST cluster (not in UniGene) | 2.89 | OVCA-R, HT29, MB-MDA-231 |
| 304777 | AA581692 | Hs.2186 | eukaryotic translation elongation factor | 2.89 | OVCA-R, EB, MCF7 |
| 337768 | | | CH22_EM:AC000097.GENSCAN.119-6 | 2.88 | NCI-H69, LnCap, DU145 |
| 319465 | AA319115 | Hs.191558 | ESTs | 2.88 | NCI-H460, NCI-H520, NCI-358 |
| 319068 | W93011 | Hs.110155 | ESTs | 2.87 | BT474, MB-MDA-453, MB-MDA-435s |
| 330958 | H08815 | Hs.159824 | EST | 2.87 | OVCA-R, PC3, A549 |
| 334215 | | | CH22_FGENES.357_7 | 2.87 | NCI-H69, PRSC_con, PRSC_log |
| 333568 | | | CH22_FGENES.185_1 | 2.87 | PRSC_con, PRSC_log, NCI-H69 |
| 333142 | | | CH22_FGENES.85_5 | 2.87 | NCI-H69, HT29, HT29 |
| 330239 | | | CH.05_p2 gi\|6671857 | 2.87 | MB-MDA-453, MB-MDA-453, EB |
| 302120 | R55140 | Hs.31075 | ESTs; Weakly similar to Weak similarity | 2.87 | CALU6, MB-MDA-435s, BT474 |
| 338679 | | | CH22_EMAC005500.GENSCAN.470-1 | 2.86 | NCI-H345, PRSC_log, PRSC_con |
| 329041 | | | CH.X_hs gi\|5868564 | 2.86 | LnCap, PRSC_con, RPWE-2 |
| 333541 | | | CH22_FGENES.178_3 | 2.86 | NCI-H69, NCI-H345, PRSC_con |
| 337011 | | | CH22_FGENES.427-6 | 2.86 | NCI-H69, PRSC_log, PRSC_con |
| 324031 | AA375646 | | EST cluster (not in UniGene) | 2.86 | NCI-H345, PRSC_log, LnCap |
| 331842 | AA416586 | Hs.98232 | ESTs | 2.86 | DU145, OVCA-R, HT29 |
| 336599 | | | CH22_FGENES.350_3 | 2.85 | LnCap, NCI-H69, NCI-H345 |
| 337586 | | | CH22_C20H12.GENSCAN.5-4 | 2.85 | NCI-H345, NCI-H69, PRSC_con |
| 336177 | | | CH22_FGENES.712_2 | 2.85 | NCI-H69, PRSC_log, RPWE-2 |
| 337522 | | | CH22_FGENES.819-1 | 2.85 | CALU6, OVCA-R, HT29 |
| 338598 | | | CH22_EM:AC005500.GENSCAN.437-2 | 2.85 | NCI-H69, PRSC_con, NCI-H345 |
| 309522 | AW150044 | Hs.252259 | ribosomal protein S3 | 2.85 | MB-MDA-453, MB-MDA-435s, MB-MDA-435s |
| 336981 | | | CH22_FGENES.397-7 | 2.85 | NCI-H69, PRSC_con, PRSC_log |
| 330286 | | | CH.05_p2 gi\|6671913 | 2.84 | NCI-H345, PRSC_log, NCI-H69 |
| 333713 | | | CH22_FGENES.251_2 | 2.84 | RPWE-2, PRSC_con, NCI-H69 |
| 335068 | | | CH22_FGENES.483_5 | 2.83 | MB-MDA-231, NCI-H345, RPWE-2 |
| 305075 | AA641288 | Hs.181165 | eukaryotic translation elongation factor | 2.83 | EB, LnCap, DU145 |
| 326380 | | | CH.19_hs gi\|5867327 | 2.82 | NCI-H69, PRSC_con, PRSC_log |
| 334970 | | | CH22_FGENES.466_3 | 2.82 | PRSC_con, NCI-H69, RPWE-2 |
| 337097 | | | CH22_FGENES.471-1 | 2.82 | NCI-H345, NCI-H69, PRSC_log |
| 323676 | AI702835 | | EST cluster (not in UniGene) | 2.82 | LnCap, A549, CALU6 |
| 333785 | | | CH22.FGENES.274_4 | 2.82 | OVCA-R, Caco2, MB-MDA-453 |
| 334175 | | | CH22.FGENES.349_10 | 2.81 | RPWE-2, BT474, MCF7 |
| 337865 | | | CH22_EM:AC005500.GENSCAN.46-5 | 2.81 | Caco2, NCI-H23, BT474 |
| 302585 | AA083564 | Hs.249220 | H sapiens mRNA for hTbr2; complete cds | 2.81 | EB, DU145, MB-MDA-453 |
| 336623 | | | CH22_FGENES.4-5 | 2.81 | NCI-H345, PRSC_con, NCI-H69 |
| 332854 | | | CH22.FGENES.22_1 | 2.8 | RPWE-2, PRSC_log, PRSC_con |
| 336978 | | | CH22.FGENES.384-10 | 2.8 | PRSC_con, NCI-H345, RPWE-2 |
| 326874 | | | CH.20_hs gi\|6682507 | 2.8 | RPWE-2, NCI-H345, PRSC_log |
| 315121 | AA585011 | Hs.105902 | ESTs | 2.8 | NCI-H345, PRSC_log, RPWE-2 |
| 311185 | AI638294 | Hs.224665 | ESTs | 2.8 | NCI-H69, NCI-H345, PRSC_log |
| 334682 | | | CH22_FGENES.419_4 | 2.8 | NCI-H69, PRSC_log, RPWE-2 |
| 316845 | AW418715 | Hs.250388 | ESTs | 2.79 | RPWE-2, NCI-H345, PRSC_log |
| 331599 | N74826 | Hs.50535 | ESTs | 2.79 | A549, MB-MDA-453, MB-MDA-435s |
| 315681 | AW022054 | Hs.136591 | ESTs | 2.78 | NCI-H460, MB-MDA-453, MCF7 |
| 313012 | AI207390 | Hs.143929 | ESTs | 2.78 | DU145, MS-MDA-453, MCF7 |
| 313476 | AA010267 | | EST cluster (not in UniGene) | 2.78 | NCI-H520, NCI-H460, HT29 |
| 327277 | | | CH.01_hs gi\|5867473 | 2.78 | DU145, CALU6, EB |
| 310981 | AI494514 | Hs.171380 | ESTs | 2.78 | LnCap, RPWE-2, NCI-H460 |
| 335090 | | | CH22.FGENES.490_1 | 2.77 | NCI-H69, PRSC_log, PRSC_con |
| 328581 | | | CH.07.hs gi\|6006033 | 2.77 | HT29, MB-MDA-453, MCF7 |
| 333219 | | | CH22_FGENES.104_11 | 2.77 | NCI-H69, PRSC_log, NCI-H345 |
| 308311 | AI581855 | | EST singleton (not in UniGene) with exon | 2.77 | MB-MDA-231, HT29, CALU6 |
| 329760 | | | CH.14_p2 gi\|6048280 | 2.77 | CALU6, DU145, ES |
| 303925 | AW469999 | Hs.258523 | ESTs | 2.77 | NCI-H69, LnCap, MS-MDA-231 |
| 337628 | | | CH22_C20H12.GENSCAN.28-31 | 2.77 | NCI-H69, LnCap, MB-MDA-453 |
| 333520 | | | CH22_FGENES.174_3 | 2.77 | NCI-H69, NCI-H345, PRSC_con |
| 303168 | AA872479 | Hs.197770 | ESTs; Weakly similar to estrogen-respons | 2.76 | DU145, OVCA-R, MB-MDA-453 |
| 313451 | AW138189 | Hs.122672 | ESTs | 2.76 | OVCA-R, EB, DU145 |
| 328474 | | | CH.07_hs gi\|5868446 | 2.76 | NCI-H69, NCI-H345, RPWE-2 |
| 331988 | AA477414 | Hs.9242 | purine-rich element binding protein B | 2.76 | MB-MDA-435s, A549, OVCA-R |
| 306180 | AA922503 | | EST singleton (not in UniGene) with exon | 2.76 | NCI-H69, DU145, LnCap |
| 321071 | AA013011 | Hs.241502 | Cdc42 effector protein 4 | 2.76 | PRSC_log, PRSC_con, NCI-H345 |
| 302972 | W73400 | | EST | 2.76 | NCI-H345, RPWE-2, NCI-H69 |
| 305185 | AA663985 | Hs.248038 | major histocompatibility complex; class | 2.75 | DU145, A549, BT474 |
| 335998 | | | CH22_FGENES.656_16 | 2.75 | NCI-H69, PRSC_con, RPWE-2 |
| 319138 | R11699 | Hs.73818 | ubiquinol-cytochrome c reductase hinge p | 2.75 | NCI-H345, NCI-H69, PRSC_con |
| 336387 | | | CH22_FGENES.822_7 | 2.75 | PRSC_con, RPWE-2, PRSC_log |
| 338054 | | | CH22.EM:AC005500.GENSCAN.158-2 | 2.75 | OVCA-R, EB, DU145 |
| 316041 | AA719183 | | EST duster (not in UniGene) | 2.74 | DU145, MCF7, MB-MDA-453 |
| 336863 | | | CH22_FGENES.297-4 | 2.74 | MB-MDA-453, MCF7, OVCA-R |
| 335975 | | | CH22_FGENES.652_9 | 2.74 | CALU6, EB, A549 |
| 302952 | AF103179 | | EST | 2.74 | CALU6, MB.MDA-435s, BT474 |
| 326122 | | | CH.17_hs gi\|5867194 | 2.74 | HT29, Caco2, PC3 |
| 337427 | | | CH22_FGENES.761-4 | 2.74 | RPWE-2, NCI-H89, PRSC_log |
| 308063 | AI469244 | Hs.119252 | tumor protein; translationally-controlle | 2.74 | NCI-358, NCI-H23, Caco2 |
| 325433 | | | CH.12_hs gi\|5866936 | 2.74 | NCI-H345, PRSC_con, RPWE-2 |
| 316252 | AI572633 | Hs.190406 | ESTs | 2.74 | OVCA-R, MCF7, A549 |
| 310837 | AI418688 | Hs.170301 | ESTs | 2.74 | NCI-H345, PRSC_con, RPWE-2 |
| 313562 | AW467335 | Hs.257676 | ESTs | 2.74 | HT29, MCF7, MB-MDA-231 |
| 335455 | | | CH22_FGENES.562_15 | 2.74 | NCI-H69, LnCap, PRSC_con |
| 304792 | AA583101 | Hs.29797 | ribosomal protein L10 | 2.73 | EB, OVCA-R, MB-MDA-453 |
| 331979 | AA469937 | Hs.105322 | EST | 2.73 | MCF7, BT474, NCI-H460 |
| 336198 | | | CH22_PGENES.719_2 | 2.73 | NCI-H69, PRSC_con, PRSC_log |
| 314698 | AI660452 | Hs.187127 | ESTs | 2.73 | MB-MDA-231, LnCap, BT474 |
| 307954 | AI419692 | | EST singleton (not in UniGene) with exon | 2.73 | HT29, HT29, EB |
| 318288 | AI088590 | Hs.134702 | ESTs | 2.73 | PRSC_log, NCI-H345, PRSC_con |
| 327833 | | | CH.05_hs gi\|5867968 | 2.73 | BT474, PC3, MB-MDA-231 |
| 300221 | AW449602 | Hs.217953 | ESTs; Highly similar to NK-TUMOR RECOGNI | 2.73 | NCI-H520, NCI-358, MB-MDA-453 |
| 326039 | | | CH.17_hs gi\|5867179 | 2.73 | MB-MDA-453, EB, ES |
| 318457 | AI149678 | Hs.143952 | ESTs | 2.72 | PRSC_con, PRSC_log, NCI-H345 |
| 336753 | | | CH22_FGENES.128-9 | 2.72 | MB-MDA-435s, NCI-H520, MCF7 |
| 330086 | | | CH.194_p2 gi\|6015293 | 2.72 | HT29, MB-MDA-453, MCF7 |
| 333566 | | | CH22_FGENES.183_2 | 2.72 | HT29, BT474, OVCA-R |
| 339384 | | | CH22_BA232E17.GENSCAN.3-22 | 2.71 | NCI-H69, NCI-H345, PRSC_log |
| 338668 | | | CH22_EMAC005500.GENSCAN.465-1 | 2.71 | NCI-H69, RPWE-2, PRSC_con |
| 300798 | AI382618 | Hs.194813 | ESTs | 2.71 | PRSC_con, NCI-H345, PRSC_log |
| 303745 | AI142379 | | EST | 2.71 | PRSC_log, PRSC_con, RPWE-2 |
| 305197 | AA666301 | | EST singleton (not in UniGene) with exon | 2.71 | EB, NCI-H520, OVCA-R |
| 338725 | | | CH22_EM:AC005500.GENSCAN.499-1 | 2.7 | OALU6, MB-MDA-453, PC3 |
| 307799 | AI351112 | | EST singleton (not in UniGene) with exon | 2.7 | HT29, BT474, MCF7 |
| 309598 | AW173642 | Hs.250106 | EST | 2.69 | NCI-358, NCI-H69, NCI-H23 |
| 302727 | L10141 | | EST | 2.69 | OVCA-R, BT474, PC3 |
| 308544 | AI695133 | | EST singleton (not in UniGene) with exon | 2.69 | HT29, CALU6, MB-MDA-435s |
| 322877 | AA079727 | | EST duster (not in UniGene) | 2.69 | NCI-H345, NCI-H69, PRSC_con |
| 325695 | | | CH.14_hs gi\|6552446 | 2.69 | NCI-H69, NCI-H460, NCI-H460 |
| 307728 | AI335557 | | EST singleton (not in UniGene) with exon | 2.68 | NCI-H69, PRSC_log, NCI-358 |
| 302399 | N79624 | | EST | 2.68 | NCI-H69, PRSC_con, NCI-H345 |
| 309343 | AW028652 | | EST singleton (not in UniGene) with exon | 2.68 | HT29, MB-MDA-231, MB-MDA-231 |
| 339360 | | | CH22_BA354I12.GENSCAN.32-2 | 2.68 | NCI-H69, PRSC_log, PRSC_con |
| 337821 | | | CH22_EMAC005500.GENSCAN.13-11 | 2.68 | PRSC_con, PRSC_log, PRSC_log |
| 337338 | | | CH22_FGENES.717-7 | 2.66 | NCI-H69, PRSC_con, PRSC_log |
| 334510 | | | CH22_FGENES.398_8 | 2.68 | NCI-H460, NCI-H23, NCI-358 |
| 300918 | AA491286 | Hs.128792 | ESTs | 2.68 | MB-MDA-435s, CALU6, DU145 |
| 335536 | | | CH22_FGENES.574_2 | 2.67 | NCI-H69, NCI-H345, PRSC_log |
| 335311 | | | CH22_FGENES.532_4 | 2.67 | MB-MDA-435s, Caco2, A549 |
| 338959 | | | CH22_DJ32I10.GENSCAN.23-31 | 2.67 | NCI-H345, PRSC_con, NCI-H69 |
| 339081 | | | CH22_DA59H18.GENSCAN.37-10 | 2.67 | NCI-H345, RPWE-2, NCI-H69 |
| 334068 | | | CH22_FGENES.327_23 | 2.67 | PRSC_con, RPWE-2, PRSC_log |
| 338976 | | | CH22_DAS9HI8.GENSCAN.1-3 | 2.66 | PRSC_con, PRSC_log, RPWE-2 |
| 325524 | | | CH.12_hs gi\|5866981 | 2.66 | NCI-H345, RPWE-2, PRSC_con |
| 333069 | | | CH22_FGENES.76_5 | 2.66 | NCI-H69, NCI-H345, PRSC_con |
| 336203 | | | CH22_FGENES.719_7 | 2.66 | OVCA-R, PC3, A549 |
| 333133 | | | CH22_FGENES.83_9 | 2.66 | HT29, OVCA-R, A549 |
| 304074 | T77842 | Hs.142528 | ESTs | 2.65 | DU145, CALU6, EB |
| 330919 | AA224594 | Hs.86941 | ESTs | 2.65 | PRSC_con, RPWE-2, LnCap |
| 333248 | | | CH22_FGENES.115_5 | 2.65 | NCI-H345, PRSC_con, MB-MDA-231 |
| 336665 | | | CH22_FGENES.42-2 | 2.65 | NCI-H69, PRSC_log, PRSC_con |
| 315322 | AA770599 | | EST cluster (not in UniGene) | 2.65 | A549, MB-MDA-453, MB-MDA-435s |
| 307474 | AI264023 | | EST singleton (not in UniGene) with exon | 2.65 | NCI-H69, NCI-H345, RPWE-2 |
| 320221 | AL050020 | Hs.127384 | DKFZPS64C196 protein | 2.65 | MB-MDA-453, MCF7, HT29 |
| 301767 | AW361892 | | EST | 2.65 | NCI-H345, PRSC_con, PRSC_log |
| 327246 | | | CH.01_hs gi\|5867547 | 2.65 | EB, OVCA-R, DU145 |
| 337403 | | | CH22_FGENES.752-2 | 2.65 | PRSC_con, PRSC_log, RPWE-2 |
| 328221 | | | CH.06_hs gi\|5868099 | 2.64 | MCF7, MB-MDA-231, BT474 |
| 336759 | | | CH22_FGENES.133-2 | 2.64 | NCI-H69, PRSC_log, PRSC_con |
| 327532 | | | CH.02_hs gi\|6469818 | 2.64 | PC3, CALU6, A549 |
| 305621 | AA789095 | | EST singleton (not in UniGene) with exon | 2.64 | HT29, MB-MDA-231, MB-MDA-453 |
| 322931 | AA099329 | Hs.151784 | ESTs | 2.64 | PRSC_con, RPWE-2, NCI-H345 |
| 327278 | | | CH.01_hs gi\|5867473 | 2.64 | EB, NCI-H460, NCI-H69 |
| 332235 | N51413 | Hs.109284 | ESTs | 2.64 | DU145, EB, OVCA-R |
| 332792 | | | CH22_FGENES.3_2 | 2.63 | HT29, Caco2, A549 |
| 312340 | A1862668 | Hs.176333 | ESTs | 2.63 | NCI-358, NCI-358, HT29 |
| 337484 | | | CH22_FGENES.795-8 | 2.63 | NCI-H69, NCI-H345, PRSC_con |
| 325783 | | | CH.14_hs gi\|6456780 | 2.63 | EB, OVCA-R, PC3 |
| 303672 | AW502380 | Hs.210527 | ESTs | 2.63 | PRSC_log, NCI-H345, NCI-H69 |
| 306009 | AA894560 | | EST singleton (not in UniGene) with exon | 2.63 | HT29, MB-MDA-231, CALU6 |
| 308548 | AI695484 | | EST singleton (not in UniGene) with exon | 2.63 | PC3, A549, NCI-358 |
| 337930 | | | CH22_EM:AC005500.GENSCAN.81-3 | 2.62 | PC3, OVCA-R, MCF7 |
| 327791 | | | CH.05_hs gi\|5867977 | 2.62 | PRSC_log, PRSC_con, NCI-H345 |
| 330925 | AA232678 | Hs.87073 | ESTs | 2.62 | OVCA-R, MCF7, LnCap |
| 327259 | | | CH.01_hs gi\|5867454 | 2.62 | NCI-H345, PRSC_con, RPWE-2 |
| 302150 | AF061756 | Hs.152531 | heart and neural crest derivatives expre | 2.61 | OVCA-R, PC3, A549 |
| 304881 | AA598501 | Hs.195188 | glyceraldehyde-3-phosphate dehydrogenase | 2.61 | MB-MDA-435s, NCI-H23, MCF7 |
| 335956 | | | CH22_FGENES.647_3 | 2.61 | DU145, PRSC_con, PC3 |
| 326506 | | | CH.19_hs gi\|5867435 | 2.61 | RPWE-2, NCI-H460, NCI-358 |
| 335863 | | | CH22_FGENES.629_8 | 2.61 | PC3, HT29, NCI-358 |
| 334752 | | | CH22_FGENES.428_1 | 2.61 | PRSC_con, NCI-H69, PRSC_log |
| 333288 | | | CH22_FGENES.128_19 | 2.61 | HT29, NCI-358, Caco2 |
| 306709 | AI024215 | Hs.131477 | EST | 2.61 | MB-MDA-435s, MCF7, BT474 |
| 305816 | AA854776 | | EST singleton (not in UniGene) with exon | 2.6 | MB-MDA-453, MCF7, MB-MDA-435s |
| 327264 | | | CH.01_hs gi\|5867461 | 2.6 | MB-MDA-435s, MB-MDA-435s, MB-MDA-453 |
| 310905 | AW075527 | Hs.252259 | ribosomal protein S3 | 2.6 | OVCA-R, EB, DU145 |
| 324492 | AA479507 | Hs.135179 | ESTs | 2.6 | DU145, EB, OVCA-R |
| 322649 | AA526549 | | EST cluster (not in UniGene) | 2.6 | PRSC_con, RPWE-2, PRSC_log |
| 329384 | | | CH.X_hs gi\|5868869 | 2.6 | NCI-H69, NCI-H345, PRSC_con |
| 321240 | M62378 | | EST duster (not in UniGene) | 2.6 | BT474, CALU6, MB-MDA-231 |
| 302751 | AA299576 | Hs.156110 | Immunoglobulin kappa variable 1D-8 | 2.59 | MCF7, MB-MDA-453, OVCA-R |
| 305841 | AA860348 | | EST singleton (not in UniGene) with exon | 2.59 | NCI-H345, PRSC_log, PRSC_con |
| 324180 | AA402242 | Hs.122799 | ESTs | 2.58 | EB, PC3, HT29 |
| 334196 | | | CH22_FGENES.353_4 | 2.58 | NCI-H345, NCI-H69, PRSC_con |
| 338451 | | | CH22_EM:AC005500.GENSCAN.359-39 | 2.58 | MB-MDA-435s, NCI-H23, MCF7 |
| 300333 | AW297396 | Hs.227052 | ESTs | 2.58 | PRSC_con, PRSC_log, NCI-H69 |
| 305046 | AA632201 | | EST singleton (not in UniGene) with exon | 2.58 | NCI-H460, MB-MDA-453, MB-MDA-435s |
| 305648 | AA807652 | Hs.156110 | Immunoglobulin kappa variable 1D-8 | 2.57 | PRSC_con, RPWE-2, NCI-H345 |
| 301744 | W22230 | | EST | 2.57 | PRSC_con, PRSC_log, NCI-H345 |
| 329182 | | | CH.X_hs gi\|6056331 | 2.57 | PRSC_con, RPWE-2, NCI-H345 |
| 318178 | AW137425 | Hs.158401 | ESTs | 2.57 | MB-MDA-231, PRSC_con, BT474 |
| 330057 | | | CH.17_p2 gi\|6478962 | 2.57 | NCI-H345, RPWE-2, PRSC_con |
| 326552 | | | CH.19._hs gi\|5867308 | 2.57 | NCI-H345, PRSC_con, RPWE-2 |
| 311956 | T67085 | Hs.188484 | ESTs | 2.57 | HT29, MB-MDA-453, NCI-H460 |
| 327185 | | | CH.01_hs gi\|6117805 | 2.57 | CALU6, HT29, EB |
| 302183 | NM_00224 | | EST | 2.57 | MCF7, PC3, OVCA-R |
| 327263 | | | CH.01_hs gi\|6525274 | 2.56 | PRSC_con, NCI-H69, PRSC_log |
| 339164 | | | CH22.DA59H18.GENSCAN.69-4 | 2.56 | NCI-H69, PRSC_con, NCI-H345 |
| 332763 | AA063554 | Hs.90959 | ESTs | 2.58 | RPWE-2, NCI-H345, PRSC_con |
| 330579 | U67733 | Hs.154437 | phosphodiesterase 2A; cGMP-stimulated | 2.55 | HT29, CALU6, PC3 |
| 329948 | | | CH.16_p2 gi\|5540101 | 2.55 | NCI-H460, MCF7, MB-MDA-453 |
| 300282 | AW044305 | Hs.236131 | ESTs; Highly similar to homeodomain-inte | 2.55 | NCI-H460, NCI-H23, NCI-H23 |
| 335448 | | | CH22_FGENES.562_5 | 2.55 | MB-MDA-453, BT474, MCF7 |
| 330959 | H09174 | Hs.26484 | HIRA-interacting protein 3 | 2.55 | MB-MDA-453, HT29, MCF7 |
| 307262 | AI202100 | | EST singleton (not in UniGene) with exon | 2.55 | MCF7, DU145, MB-MDA-435s |
| 335806 | | | CH22_FGENES.616_8 | 2.55 | NCI-H345, NCI-H69, PRSC_con |
| 335782 | | | CH22_FGENES.609_4 | 2.55 | Caco2, MB-MDA-453, MB-MDA-435s |
| 301703 | AW301478 | | EST | 2.55 | PC3, MCF7, MB-MDA-453 |
| 329018 | | | CH.X_hs gi\|6249620 | 2.54 | NCI-H69, PRSC_log, PRSC_con |
| 329870 | | | CH.14_p2 gi\|6706435 | 2.54 | NCI-H23, NCI-H460, NCI-358 |
| 334504 | | | CH22_FGENES.398_2 | 2.54 | HT29, BT474, MB.MDA-231 |
| 304707 | AA564846 | | EST singleton (not in UniGene) with exon | 2.53 | NCI-H520, EB, NCI-H460 |
| 329326 | | | CH.X_hs gi\|5868806 | 2.53 | MB-MDA-231, NCI-H345, NCI-H69 |
| 334418 | | | CH22_FGENES.384_5 | 2.53 | NCI-H23, NCI-358, NCI-H460 |
| 338124 | | | CH22_EM:AC005500.GENSCAN.196-2 | 2.53 | NCI-H69, PRSC_con, PRSC_log |
| 318423 | AI362671 | Hs.214491 | ESTs | 2.53 | OVCA-R, EB, DU145 |
| 333006 | | | CH22_FGENES.60_3 | 2.53 | NCI-H69, PRSC_con, PRSC_log |
| 333668 | | | CH22_FGENES.245_2 | 2.53 | NCI-H69, PRSC_log, PRSC_con |
| 333567 | | | CH22_FGENES.184_2 | 2.63 | NCI-H69, NCI-H345, PRSC_con |
| 309592 | AW172384 | | EST singleton (not in UniGene) with exon | 2.52 | LnCap, NCI-H69, DU145 |
| 328989 | | | CH.09_hs gi\|5868535 | 2.52 | MB-MDA-435s, OVCA-R, EB |
| 326725 | | | CH.20_hs gi\|6552456 | 2.52 | PRSC_con, NCI-H345, NCI-H69 |
| 302996 | AF054863 | | EST | 2.52 | HT29, BT474, CALU6 |
| 335733 | | | CH22_FGENES.601_3 | 2.52 | NCI-H69, PRSC_log, NCI-H345 |
| 336000 | | | CH22_FGENES.658_1 | 2.52 | LnCap, OVCA-R, DU145 |
| 327774 | | | CH.05_hs gi\|5867964 | 2.52 | DU145, CALU6, HT29 |
| 328557 | | | CH.07_hs gi\|5868489 | 2.52 | MB-MDA-453, MB-MDA-435s, MCF7 |
| 328228 | | | CH.06_hs gi\|5868105 | 2.52 | NCI-H69, NCI-H345, PRSC_con |
| 328305 | | | CH.07_hs gi\|6004478 | 2.52 | NCI-H69, NCI-H460, PRSC_log |
| 334010 | | | CH22_FGENES.313_1 | 2.51 | NCI-H69, PRSC_log, PRSC_con |
| 339033 | | | CH22_DA59H18.GENSCAN.26-1 | 2.51 | NCI-H69, NCI-H345, PRSC_con |
| 335340 | | | CH22_FGENES.535_17 | 2.51 | NCI-H69, PRSC_con, PRSC_log |
| 300156 | AI245582 | Hs.233395 | ESTs | 2.51 | PRSC_con, PRSC_log, NCI-H345 |
| 305880 | AA866065 | Hs.156110 | Immunoglobulin kappa variable 1D-8 | 2.5 | EB, OVCA-R, DU145 |
| 310841 | AI968009 | Hs.232024 | ESTs | 2.5 | LnCap, NCI-358, CALU6 |
| 336908 | | | CH22_FGENES.343-2 | 2.5 | NCI-H345, RPWE-2, PRSC_log |
| 304674 | AA541735 | | EST singleton (not in UniGene) with exon | 2.5 | RPWE-2, NCI-H69, MCF7 |
| 314521 | AW503939 | Hs.107149 | ESTs; Weakly similar to PTB-ASSOCIATED S | 2.5 | NCI-H460, EB, Caco2 |
| 307592 | AI285739 | | EST singleton (not in UniGene) with exon | 2.5 | PRSC_con, NCI-H345, PRSC_log |
| 331476 | N26190 | Hs.43768 | ESTs | 2.5 | NCI-H345, NCI-H69, PRSC_con |
| 325803 | | | CH.14_hs gi\|6552451 | 2.5 | NCI-H345, RPWE-2, PRSC_con |
| 306549 | AA993796 | | EST singleton (not in UniGene) with exon | 2.49 | A549, OVCA-R, CALU6 |
| 304833 | AA586504 | | EST singleton (not in UniGene) with exon | 2.49 | MCF7, DU145, LnCap |
| 336333 | | | CH22_FGENES.813.1 | 2.49 | NCI-H345, PRSC_con, PRSC_log |
| 332320 | T71134 | Hs.100551 | EST | 2.49 | NCI-H345, LnCap, RPWE-2 |
| 328236 | | | CH.06_hs gi\|5868117 | 2.49 | PRSC_con, NCI-H345, PRSC_log |
| 317335 | AI656979 | Hs.130210 | ESTs | 2.49 | MCF7, MB-MDA-453, PC3 |
| 339188 | | | CH22_DA59H18.GENSCAN.72-16 | 2.48 | NCI-H69, PRSC_con, PRSC_log |
| 334235 | | | CH22_FGENES.361_19 | 2.48 | NCI-H520, MB-MDA-453, A549 |
| 301214 | AW450950 | Hs.157034 | ESTs; Weakly similar to Unknown [H.sapie | 2.48 | HT29, A549, A549 |
| 332843 | | | CH22_FGENES.19_1 | 2.48 | DU145, CALU6, EB |
| 337431 | | | CH22_FGENES.763-7 | 2.48 | PRSC_con, RPWE-2, NCI-H69 |
| 336757 | | | CH22_FGENES.131-1 | 2.48 | NCI-H69, PRSC_log, PRSC_con |
| 305403 | AA723748 | | EST singleton (not in UniGene) with exon | 2.48 | NCI-H23, DU145, OVCA-R |
| 330065 | | | CH.19_p2 gi\|6165044 | 2.48 | PRSC_con, PRSC_log, NCI-H69 |
| 309245 | AI972447 | | EST singleton (not in UniGene) with exon | 2.48 | MB-MDA-231, NCI-H69, HT29 |
| 328876 | | | CH.07_hs gi\|6525286 | 2.47 | MB-MDA-231, CALU6, PC3 |
| 333944 | | | CH22_FGENES.302_2 | 2.47 | NCI-H69, RPWE-2, PRSC_log |
| 328504 | | | CH.07_hs gi\|5868471 | 2.47 | LnCap, MB-MDA-453, MB-MDA-435s |
| 336120 | | | CH22_EM:AC005500.GENSCAN.195-1 | 2.47 | MB-MDA-231, NCI-H69, PRSC_con |
| 306710 | AI024221 | | EST singleton (not in UniGene) with exon | 2.47 | OVCA-R, EB, LnCap |
| 305064 | AA636012 | | EST singleton (not in UniGene) with exon | 2.47 | NCI-H69, RPWE-2, PRSC_con |
| 329995 | | | CH.16_p2 gi\|4567166 | 2.47 | OVCA-R, DU145, MB-MDA-453 |
| 315694 | AI821743 | Hs.168418 | ESTs; Moderately similar to !!!! ALU SUB | 2.46 | EB, A549, LnCap |
| 331004 | H64622 | Hs.32748 | ESTs | 2.46 | EB, MCF7, MB-MDA-435s |
| 305259 | AA679225 | | EST singleton (not in UniGene) with exon | 2.46 | PRSC_con, NCI-H345, RPWE-2 |
| 304576 | AA496563 | | EST singleton (not in UniGene) with exon | 2.46 | PRSC_con, RPWE-2, PRSC_log |
| 318887 | R60487 | Hs.21065 | ESTs | 2.46 | NCI-H345, Caco2, Caco2 |
| 308954 | AI868958 | | EST singleton (not in UniGene) with exon | 2.46 | PRSC_con, PRSC_log, RPWE-2 |
| 301140 | AI807692 | Hs.207128 | ESTs | 2.46 | OVCA-R, MB-MDA-231, HT29 |
| 322085 | AA088500 | Hs.170298 | ESTs | 2.46 | PRSC_log, PRSC_con, NCI-H345 |
| 339130 | | | CH22_DA59H18.GENSCAN.56-3 | 2.46 | NCI-H345, PRSC_con, RPWE-2 |
| 337612 | | | CH22_C20H12.GENSCAN.22-5 | 2.46 | EB, A549, Caco2 |
| 313765 | AW206181 | Hs.185981 | ESTs; Weakly similar to gag [H.sapiens] | 2.45 | RPWE-2, PRSC_log, PRSC_con |
| 311665 | AW294254 | Hs.223742 | ESTs | 2.45 | PRSC_log, RPWE-2, PRSC_con |
| 328620 | | | CH.07.hs gi\|5868241 | 2.45 | MB-MDA-453, MCF7, MB-MDA-435s |
| 305361 | AA708902 | | EST singleton (not in UniGene) with exon | 2.45 | HT29, MB-MDA-435s, A549 |
| 336243 | | | CH22_FGENES.746_1 | 2.44 | OVCA-R, MB-MDA-453, MB-MDA-435s |
| 320299 | H08323 | Hs.177181 | ESTs | 2.44 | PRSC_con, RPWE-2, NCI-H345 |
| 302535 | H48676 | | EST | 2.44 | MB-MDA-453, EB, DU145 |
| 333465 | | | CH22_FGENES.160_2 | 2.44 | NCI-H69, PRSC_con, PRSC_log |
| 334109 | | | CH22_FGENES.330_8 | 2.44 | NCI-H69, NCI-H345, PRSC_log |
| 301749 | F12998 | Hs.90790 | ESTs | 2.44 | NCI-H345, RPWE-2, PRSC_log |
| 324575 | AW502257 | | EST cluster (not in UniGene) | 2.44 | NCI-H345, PRSC_con, RPWE-2 |
| 337114 | | | CH22_FGENES.494-17 | 2.44 | NCI-H69, PRSC_log, PRSC_con |
| 336087 | | | CH22_FGENES.688_16 | 2.44 | PRSC_con, Caco2, PRSC_log |
| 315678 | AI657119 | Hs.120036 | ESTs | 2.44 | NCI-358, PC3, NCI-H23 |
| 333258 | | | CH22.FGENES.118_6 | 2.44 | MB-MDA-231, HT29, CALU6 |
| 303798 | V00505 | Hs.36977 | hemoglobin; delta | 2.44 | MB-MDA-435s, MCF7, MB-MDA-453 |
| 309759 | AW268822 | | EST singleton (not in UniGene) with exon | 2.44 | MB-MDA-453, EB, MCF7 |
| 318946 | AI122843 | | EST cluster (not in UniGene) | 2.44 | PC3, OVCA-R, DU145 |
| 321986 | AL133656 | | EST cluster (not in UniGene) | 2.44 | DU145, CALU6, CALU6 |
| 336151 | | | CH22_EM:AC005500.GENSCAN.207-5 | 2.44 | PRSC_con, PRSC_log, RPWE-2 |
| 327056 | | | CH.21_hs gi\|6531965 | 2.44 | PRSC_con, NCI-H345, RPWE-2 |
| 309605 | AW182800 | | EST singleton (not in UniGene) with exon | 2.43 | NCI-358, NCI-H23, NCI-H520 |
| 335783 | | | CH22_FGENES.610_3 | 2.43 | PRSC_con, PRSC_log, NCI-H345 |
| 325790 | | | CH.14_hs gi\|6381957 | 2.43 | MB-MDA-435s, MB-MDA-453, MB-MDA-453 |
| 339342 | | | CH22_BA354I12, GENSCAN.27-10 | 2.43 | BT474, MB-MDA-231, MB-MDA-453 |
| 335777 | | | CH22_FGENES.607_13 | 2.43 | DU145, EB, BT474 |
| 309972 | AW450350 | Hs.257283 | ESTs | 2.43 | MCF7, MB-MDA-453, OVCA-R |
| 308718 | AI798009 | | EST singleton (not in UniGene) with exon | 2.43 | NCI-H345, PRSC_con, PRSC_log |
| 338087 | | | CH22_EM:AC005500.GENSCAN.174-16 | 2.43 | DU145, PC3, CALU6 |
| 306930 | AI124518 | | EST singleton (not in UniGene) with exon | 2.43 | NCI-H69, MCF7, BT474 |
| 319032 | AW409728 | Hs.80449 | ESTs; Weakly similar to cytoplasmic dyne | 2.43 | RPWE-2, A549, NCI-H69 |
| 304330 | AA157834 | | EST singleton (not in UniGene) with exon | 2.43 | MB-MDA-453, PC3, OVCA-R |
| 320636 | R54766 | Hs.101120 | ESTs | 2.43 | MCF7, MB-MDA-435s, MB-MDA-453 |
| 335281 | | | CH22_FGENES.524_4 | 2.43 | PC3, LnCap, A549 |
| 317431 | AI675790 | Hs.132453 | ESTs | 2.43 | NCI-H345, RPWE-2, PRSC_log |
| 306511 | AA988891 | | EST singleton (not in UniGene) with exon | 2.43 | OVCA-R, EB, DU145 |
| 333298 | | | CH22_FGENES.133_4 | 2.43 | EB, DU145, PC3 |
| 328436 | | | CH.07_hs gi\|5868417 | 2.43 | EB, LnCap, A549 |
| 333420 | | | CH22_FGENES.146_11 | 2.43 | NCI-H345, NCI-H69, PRSC_log |
| 338113 | | | CH22_EM:AC005500.GENSCAN.188-13 | 2.42 | DU145, EB, CALU6 |
| 335188 | | | CH22_FGENES.507_3 | 2.42 | EB, A549, BT474 |
| 329164 | | | CH.X_hs gi\|5868691 | 2.42 | RPWE-2, PRSC_con, PRSC_log |
| 336316 | | | CH22_FGENES.799_11 | 2.42 | MB-MDA-435s, MCF7, NCI-H69 |
| 310831 | AI927594 | Hs.161142 | ESTs | 2.42 | NCI-H345, PRSC_con, PRSC_log |
| 327334 | | | CH.01_hs gi\|5902477 | 2.42 | MB-MDA-453, MB-MDA-435s, MCF7 |
| 334017 | | | CH22_FGENES.315_2 | 2.42 | PRSC_con, PRSC_log, RPWE-2 |
| 308138 | AI494446 | | EST singleton (not in UniGene) with exon | 2.42 | DU145, LnCap, EB |
| 333074 | | | CH22_FGENES.76_10 | 2.42 | NCI-H69, RPWE-2, PRSC_log |
| 306548 | AA993109 | | EST singleton (not in UniGene) with exon | 2.42 | HT29, CALU6, LnCap |
| 336516 | | | CH22_FGENES.836_1 | 2.42 | NCI-H69, PRSC_con, PRSC_log |
| 306791 | AI042387 | | EST singleton (not in UniGene) with exon | 2.42 | CALU6, DU145, EB |
| 329411 | | | CH.X_hs gi\|6682549 | 2.42 | OVCA-R, EB, LnCap |
| 308659 | AI750091 | | EST singleton (not in UniGene) with exon | 2.41 | EB, DU145, CALU6 |
| 313504 | AI190405 | Hs.143127 | ESTs | 2.41 | DU145, EB, CALU6 |
| 326073 | | | CH.17_hs gi\|6682495 | 2.41 | DU145, A549, MB-MDA-435s |
| 334047 | | | CH22_FGENES.326_5 | 2.41 | PRSC_con, PRSC_log, NCI-H345 |
| 325464 | | | CH.12_hs gi\|5866947 | 2.41 | NCI-358, NCI-H23, NCI-H460 |
| 334764 | | | CH22_FGENES.428_13 | 2.41 | NCI-H69, NCI-H345, RPWE-2 |
| 312737 | AI033500 | Hs.132895 | ESTs | 2.41 | OVCA-R, DU145, CALU6 |
| 306591 | AI000248 | | EST singleton (not in UniGene) with exon | 2.41 | MB-MDA-231, MCF7, DU145 |
| 333582 | | | CH22_FGENES.201_2 | 2.41 | NCI-H69, PRSC_con, PRSC_log |
| 337843 | | | CH22_EM:AC005500.GENSCAN.30-8 | 2.4 | EB, LnCap, A549 |
| 335284 | | | CH22_FGENES.526_6 | 2.4 | NCI-H69, NCI-H345, PRSC_log |
| 305134 | AA653159 | | EST singleton (not in UniGene) with exon | 2.4 | DU145, HT29, MB-MDA-453 |
| 335527 | | | CH22_FGENES.572_7 | 2.4 | DU145, OVCA-R, EB |
| 336795 | | | CH22_FGENES.176-5 | 2.4 | NCI-H69, NCI-H345, PRSC_log |
| 303144 | AF202889 | | EST | 2.4 | PRSC_con, PRSC_log, NCI-H69 |
| 334948 | | | CH22_FGENES.465_15 | 2.4 | PRSC_con, PRSC_log, RPWE-2 |
| 328860 | | | CH.07_hs gi\|6381928 | 2.4 | PRSC_con, PRSC_log, NCI-H345 |
| 322929 | AI365585 | Hs.148246 | ESTs | 2.4 | NCI-H460, A549, HT29 |
| 333561 | | | CH22_FGENES.180_18 | 2.4 | OVCA-R, EB, DU145 |
| 338239 | | | CH22_EM:AC005500.GENSCAN.264-5 | 2.4 | NCI-H69, NCI-H345, PRSC_con |
| 323670 | AL040411 | Hs.161763 | ESTs; Weakly similar to KIAA0738 protein | 2.4 | DU145, MB-MDA-453, EB |
| 305903 | AA873085 | | EST singleton (not in UniGene) with exon | 2.4 | MCF7, A549, NCI-H520 |
| 312573 | AW297673 | Hs.190526 | ESTs | 2.4 | LnCap, NCI-H460, NCI-H23 |
| 334470 | | | CH22_FGENES.394_1 | 2.4 | NCI-H520, HT29, NCI-H23 |
| 333272 | | | CH22_FGENES.122_1 | 2.39 | NCI-H345, PRSC_con, RPWE-2 |
| 304010 | AW518383 | Hs.177592 | ribosomal protein; large; P1 | 2.39 | DU145, CALU6, EB |
| 337316 | | | CH22_FGENES.692-1 | 2.39 | MCF7, BT474, OVCA-R |
| 316769 | AI914939 | Hs.212184 | ESTs | 2.39 | PRSC_con, NCI-H345, RPWE-2 |
| 336280 | | | CH22_FGENES.763_4 | 2.39 | NCI-H345, PRSC_log, PRSC_con |
| 331223 | T98872 | Hs.194181 | ESTs | 2.39 | DU145, HT29, PC3 |
| 337172 | | | CH22_FGENES.565-2 | 2.39 | EB, OVCA-R, DU145 |
| 300625 | AI671992 | Hs.143631 | ESTs; Weakly similar to WASP-family prot | 2.39 | EB, NCI-H520, LnCap |
| 337092 | | | CH22_FGENES.465-12 | 2.39 | PRSC_con, PRSC_log, NCI-H69 |
| 334528 | | | CH22_FGENES.402_8 | 2.39 | NCI-H345, PRSC_con, NCI-H69 |
| 338411 | | | CH22_EM:AC005500.GENSCAN.341-7 | 2.39 | NCI-H345, NCI-H69, PRSC_con |
| 331344 | AA357927 | Hs.70208 | ESTs | 2.39 | PC3, EB, A549 |
| 334044 | | | CH22.FGENES.323_2 | 2.38 | MB-MDA-231, MCF7, LnCap |
| 333918 | | | CH22_FGENES.296_7 | 2.38 | RPWE-2, NCI-H345, EB |
| 317168 | AI042614 | Hs.125910 | ESTs | 2.38 | NCI-H345, PRSC_con, RPWE-2 |
| 333424 | | | CH22_FGENES.147_4 | 2.38 | DU145, MCF7, OVCA-R |
| 317779 | AW450515 | Hs.128361 | ESTs | 2.38 | EB, DU145, OVCA-R |
| 315142 | AI380577 | Hs.190219 | ESTs | 2.38 | OVCA-R, EB, CALU6 |
| 310471 | AW270515 | Hs.149596 | ESTs | 2.38 | NCI-H460, NCI-H23, NCI-H23 |
| 325049 | AW410339 | Hs.256310 | ESTs; Weakly similar to centaurin beta2 | 2.38 | PRSC_con, RPWE-2, NCI-H345 |
| 305234 | AA670431 | | EST singleton (not in UniGene) with exon | 2.38 | MB-MDA-453, MB-MDA-231, A549 |
| 337760 | | | CH22_EM:AC000097.GENSCAN.116-8 | 2.38 | PRSC_con, PRSC_log, RPWE-2 |
| 311502 | AW204360 | Hs.208662 | ESTs | 2.38 | NCI-H345, NCI-H69, LnCap |
| 337548 | | | CH22_FGENES.844-5 | 2.38 | MB-MDA-453, MCF7, CALU6 |
| 326981 | | | CH.21_hs gi\|6588016 | 2.38 | NCI-H348, NCI-H69, PRSC_con |
| 309600 | AW182066 | | EST singleton (not in UniGene) with exon | 2.37 | RPWE-2, NCI-358, NCI-H69 |
| 328936 | | | CH.08_hs gi\|5868500 | 2.37 | OVCA-R, MB-MDA-453, CALU6 |
| 327937 | | | CH.06_hs gi\|5868192 | 2.37 | BT474, EB, OVCA-R |
| 328282 | | | CH.07_hs gi\|5868353 | 2.37 | DU145, CALU6, CALU6 |
| 303607 | AL046388 | Hs.208206 | ESTs; Weakly similar to Naf1 alpha prote | 2.37 | LnCap, PRSC_log, NCI-H345 |
| 304227 | N94974 | Hs.75344 | ribosomal protein S4; X-linked | 2.37 | EB, PC3, OVCA-R |
| 314101 | AW452279 | Hs.257542 | ESTs | 2.37 | OVCA-R, CALU6, CALU6 |
| 325026 | AI671168 | Hs.12285 | ESTs | 2.37 | NCI-H345, PRSC_con, PRSC_log |
| 315015 | AI659989 | Hs.132625 | ESTs | 2.37 | MB-MDA-453, MB-MDA-231, LnCap |
| 328662 | | | CH.07_hs gi\|6004473 | 2.37 | NCI-H345, RPWE-2, PRSC_con |
| 305867 | AA864572 | | EST singleton (not in UniGene) with exon | 2.37 | MCF7, MB-MDA-453, MB-MDA-231 |
| 333296 | | | CH22_FGENES.132_3 | 2.37 | EB, PC3, CALU6 |
| 331070 | R01116 | Hs.182059 | ESTs | 2.36 | OVCA-R, MB-MDA-453, A549 |
| 333698 | | | CH22_FGENES.250_12 | 2.36 | HT29, OVCA-R, Caco2 |
| 316423 | AA758756 | Hs.121380 | ESTs | 2.36 | HT29, MCF7, MB-MDA-435s |
| 323189 | AL121194 | Hs.120589 | ESTs | 2.36 | PC3, NCI-H460, DU145 |
| 318889 | Z43296 | Hs.18720 | programmed cell death 8 (apoptosis-induc | 2.36 | OVCA-R, A549, MB-MDA-453 |
| 334237 | | | CH22_FGENES.362_1 | 2.36 | NCI-H345, NCI-H69, LnCap |
| 315931 | AI700148 | Hs.117328 | ESTs | 2.36 | MCF7, NCI-H345, DU145 |
| 326884 | | | CH.20_hs gi\|668251 1 | 2.36 | A549, EB, PC3 |
| 333132 | | | CH22_FGENES.83_8 | 2.36 | NCI-H69, HT29, EB |
| 306574 | AA995719 | Hs.76067 | heat shock 27 kD protein 1 | 2.36 | RPWE-2, PRSC_log, PRSC_con |
| 324416 | AI669524 | Hs.194115 | ESTs | 2.36 | NCI-H345, RPWE-2, PRSC_con |
| 329496 | | | CH.10_p2 gi\|3983518 | 2.35 | HT29, MCF7, MB-MDA-231 |
| 320994 | H22381 | | EST cluster (not in UniGene) | 2.35 | NCI-H23, A549, CALU6 |
| 320481 | AA461139 | Hs.24372 | ESTs; Weakly similar to dJ207H1.1 [H.sap | 2.35 | PRSC_con, RPWE-2, PRSC_log |
| 309958 | AW444488 | | EST singleton (not in UniGene) with exon | 2.35 | NCI-H345, PRSC_con, PRSC_log |
| 327009 | | | CH.21_hs gi\|5867664 | 2.35 | HT29, BT474, MCF7 |
| 309594 | AW172821 | Hs.181165 | eukaryotic translation elongation factor | 2.35 | HT29, DU145, EB |
| 335468 | | | CH22_FGENES.567_4 | 2.35 | NCI-H69, PRSC_con, NCI-H345 |
| 304269 | AA069029 | | EST singleton (not in UniGene) with exon | 2.35 | PRSC_con, PRSC_log, RPWE-2 |
| 305877 | AA865649 | | EST singleton (not in UniGene) with exon | 2.35 | A549, MCF7, OVCA-R |
| 305700 | AA815428 | | EST singleton (not in UniGene) with exon | 2.35 | PRSC_con, NCI-H345, PRSC_log |
| 326423 | | | CH.19_hs gi\|5867369 | 2.34 | PC3, MCF7, LnCap |
| 334560 | | | CH22_FGENES.404_3 | 2.34 | HT29, NCI-H460, MB-MDA-435s |
| 337100 | | | CH22_FGENES.472-3 | 2.34 | PRSC_log, PRSC_con, RPWE-2 |
| 301505 | AW014374 | Hs.144849 | ESTs | 2.34 | CALU6, MB-MDA-231, DU145 |
| 312142 | AW298359 | Hs.221069 | ESTs | 2.34 | PRSC_con, RPWE-2, PRSC_log |
| 305787 | AA845035 | | EST singleton (not in UniGene) with exon | 2.34 | NCI-H23, NCI-H520, NCI-H460 |
| 338686 | | | CH22_EM:AC005500.GENSCAN.472-5 | 2.33 | BT474, MB-MDA-231, MB-MDA-453 |
| 331977 | AA465207 | Hs.125887 | ESTs | 2.33 | OVCA-R, A549, MB-MDA-435s |
| 314687 | M79114 | Hs.135177 | ESTs | 2.33 | NCI-H69, PRSC_con, NCI-H345 |
| 336089 | | | CH22_FGENES.688_18 | 2.33 | PRSC_con, Caco2, PRSC_log |
| 338952 | | | CH22_DJ32110.GENSCAN.23-22 | 2.33 | PC3, OVCA-R, HT29 |
| 334612 | | | CH22_FGENES.411_11 | 2.33 | OVCA-R, MB-MDA-453, EB |
| 338223 | | | CH22_EM:AC005500.GENSCAN.250-10 | 2.33 | DU145, MB-MDA-453, MCF7 |
| 327845 | | | CH.05_hs gi\|6531962 | 2.32 | OVCA-R, MB-MDA-453, PC3 |
| 308187 | AI538108 | Hs.156110 | Immunoglobulin kappa variable 10-8 | 2.32 | NCI-H69, NCI-358, PRSC_con |
| 317767 | AW294164 | Hs.128340 | ESTs; Weakly similar to Cdc42 GTPase-act | 2.32 | BT474, CALU6, MB-MDA-231 |
| 330408 | L10343 | Hs.112341 | protease inhibitor 3; skin-derived (SKAL | 2.32 | PC3, Caco2, HT29 |
| 319003 | R17712 | | EST cluster (not in UniGene) | 2.32 | MCF7, PC3, MB-MDA-453 |
| 323022 | AI066733 | Hs.133885 | ESTs | 2.32 | CALU6, MB-MDA-231, DU145 |
| 303148 | R73167 | Hs.127317 | ESTs; Weakly similar to CYTOCHROME P450 | 2.32 | NCI-H345, PRSC_con, RPWE-2 |
| 303215 | AW250314 | | EST | 2.32 | NCI-H345, PRSC_con, PRSC_log |
| 318891 | H10477 | Hs.196208 | ESTs; Weakly similar to !!!! ALU SUBFAMI | 2.32 | NCI-H69, LnCap, NCI-H345 |
| 336653 | | | CH22_FGENES.33-4 | 2.32 | DU145, EB, LnCap |
| 333329 | | | CH22_FGENES.138_22 | 2.32 | DU145, BT474, MB-MDA-231 |
| 301980 | U69962 | Hs.121498 | potassium voltage-gated channel; Shab-re | 2.31 | NCI-H345, MB-MDA-231, LnCap |
| 336968 | | | CH22_FGENES.375-28 | 2.31 | HT29, BT474, EB |
| 308539 | AI694191 | | EST singleton (not in UniGene) with exon | 2.31 | NCI-H345, NCI-H69, PRSC_log |
| 326417 | | | CH.19_hs gi\|5867362 | 2.31 | HT29, MCF7, BT474 |
| 328861 | | | CH.07_hs gi\|6381923 | 2.31 | NCI-H520, NCI-H460, NCI-H23 |
| 329254 | | | CH.X_hs gi\|5868733 | 2.31 | RPWE-2, NCI-H345, PRSC_con |
| 303075 | W88779 | Hs.59125 | ESTs | 2.3 | DU145, OVCA-R, EB |
| 335131 | | | CH22_FGENES.497_15 | 2.3 | NCI-H69, NCI-H345, PRSC_log |
| 303129 | AA308334 | Hs.172210 | MUF1 protein | 2.3 | LnCap, DU145, HT29 |
| 327067 | | | CH.21_hs gi\|6531965 | 2.3 | NCI-H345, NCI-H69, MB-MDA-435s |
| 324064 | AW137650 | | EST cluster (not in UniGene) | 2.3 | DU145, HT29, EB |
| 325965 | | | CH.16_hs gi\|5887147 | 2.3 | NCI-H69, NCI-H345, RPWE-2 |
| 334525 | | | CH22_FGENES.402_4 | 2.3 | NCI-H345, PRSC_con, NCI-H69 |
| 336654 | | | CH22_FGENES.34-2 | 2.3 | BT474, PC3, ME-MDA-453 |
| 302348 | AF100779 | Hs.194680 | WNT1 inducible signaling pathway protein | 2.3 | LnCap, CALU6, DU145 |
| 309275 | AI989570 | | EST singleton (not in UniGene) with exon | 2.3 | NCI-H460, NCI-H23, NCI-H520 |
| 329246 | | | CH.X_hs gi\|5868732 | 2.3 | NCI-H69, NCI-H345, PRSC_log |
| 305557 | AA774834 | | EST singleton (not in UniGene) with exon | 2.3 | CALU6, CALU6, MCF7 |
| 322907 | AA084941 | | EST cluster (not in UniGene) | 2.3 | MB-MDA-231, CALU6, EB |
| 318683 | AI703241 | Hs.202653 | ESTs; Weakly similar to Xin [M.musculus] | 2.29 | NCI-H345, PRSC_con, RPWE-2 |
| 309233 | AI971416 | | EST singleton (not in UniGene) with exon | 2.29 | CALU6, OVCA-R, EB |
| 308913 | AI860692 | Hs.119122 | ribosomal protein L13a | 2.29 | MB-MDA-435s, MCF7, HT29 |
| 335827 | | | CH22_FGENES.620_1 | 2.29 | PRSC_con, PRSC_log, RPWE-2 |
| 334066 | | | CH22_FGENES.327_21 | 2.29 | PRSC_con, PRSC_log, NCI-H345 |
| 302656 | AW293005 | Hs.220905 | ESTs | 2.29 | NCI-H23, Caco2, CALU6 |
| 308974 | AI872290 | Hs.140 | immunoglobulin gamma 3 (Gm marker) | 2.29 | CALU6, A549, NCI-H69 |
| 333607 | | | CH22_FGENES.216_2 | 2.29 | OVCA-R, MCF7, A549 |
| 335174 | | | CH22_FGENES.504_4 | 2.29 | HT29, A549, MB-MDA-453 |
| 332028 | AA489680 | Hs.134406 | ESTs; Weakly similar to Dim1p homolog [H | 2.29 | EB, A549, DU145 |
| 336417 | | | CH22_FGENES.823_39 | 2.29 | NCI-H69, NCI-H345, PRSC_log |
| 323426 | AA251401 | | EST cluster (not in UniGene) | 2.29 | HT29, MB-MDA-231, BT474 |
| 336618 | | | CH22_FGENES.2-1 | 2.29 | NCI-358, NCI-H460, NCI-H69 |
| 310017 | AI188739 | Hs.148488 | ESTs | 2.29 | NCI-H345, PRSC_log, PRSC_con |
| 334055 | | | CH22_FGENES.327_6 | 2.28 | DU145, OVCA-R, MB-MDA-453 |
| 337168 | | | CH22_FGENES.562-28 | 2.28 | NCI-H69, PRSC_log, NCI-H345 |
| 329824 | | | CH.14_p2 gi\|6630758 | 2.28 | NCI-H23, CALU6, RPWE-2 |
| 333891 | | | CH22_FGENES.292_13 | 2.28 | NCI-H69, MB-MDA-231, RPWE-2 |
| 339127 | | | CH22_DAS9H18.GENSCAN.55-1 | 2.28 | PRSC_con, NCI-H345, RPWE-2 |
| 305686 | AA812726 | | EST singleton (not in UniGene) with exon | 2.28 | NCI-H520, NCI-H23, NCI-H460 |
| 329782 | | | CH.14_p2 gi\|5912597 | 2.28 | NCI-H69, NCI-H345, PRSC_log |
| 311059 | AI810001 | Hs.175346 | ESTs | 2.28 | MCF7, BT474, MB-MDA-435s |
| 336934 | | | CH22_FGENES.351-1 | 2.28 | BT474, HT29, MB-MDA-435s |
| 314893 | AA761093 | | EST cluster (not in UniGene) | 2.28 | OVCA-R, HT29, DU145 |
| 331596 | N72574 | Hs.50220 | ESTs | 2.28 | A549, MCF7, NCI-358 |
| 330729 | AA258559 | Hs.3736 | ESTs; Weakly similar to DELTA-LIKE PROTE | 2.28 | MB-MDA-231, CALU6, MCF7 |
| 338285 | | | CH22_EM:AC005500.GENSCAN.293-3 | 2.27 | NCI-H69, PRSC_log, PRSC_con |
| 300154 | AI245127 | Hs.179331 | ESTs | 2.27 | NCI-H23, NCI-H520, NCI-358 |
| 306383 | AA969078 | Hs.183698 | ribosomal protein L29 | 2.27 | RPWE-2, NCI-H345, PRSC_log |
| 309005 | AI884454 | | EST singleton (not in UniGene) with exon | 2.27 | A549, MCF7, BT474 |
| 332995 | | | CH22_FGENES.58_2 | 2.27 | RPWE-2, NCI-H345, PRSC_log |
| 337426 | | | CH22_FGENES.761-3 | 2.27 | DU145, EB, CALU6 |
| 337778 | | | CH22_EM:AC000097.GENSCAN.119-20 | 2.27 | NCI-H69, PRSC_con, PRSC_log |
| 329705 | | | CH.14_p2 gi\|6065790 | 2.27 | PRSC_con, PRSC_log, RPWE-2 |
| 335971 | | | CH22_FGENES.652_4 | 2.27 | PRSC_log, MB-MDA-231, NCI-H23 |
| 315862 | AI075848 | Hs.133996 | ESTs | 2.27 | HT29, MB-MDA-435s, OVCA-R |
| 316466 | AI911204 | Hs.126365 | ESTs | 2.27 | NCI-8460, NCI-358, BT474 |
| 334430 | | | CH22_FGENES.385_3 | 2.27 | NCI-H345, NCI-H69, PRSC_con |
| 331941 | AA452257 | Hs.99272 | ESTs | 2.26 | PRSC_con, LnCap, PRSC_log |
| 301230 | AW269804 | Hs.153019 | ESTs | 2.26 | NCI-H345, PRSC_log, NCI-H520 |
| 317394 | AI935024 | Hs.190518 | ESTs | 2.26 | NCI-H345, PRSC_con, PRSC_log |
| 306220 | AA928363 | | EST singleton (not in UniGene) with exon | 2.26 | NCI-H345, PRSC_con, PRSC_log |
| 304134 | H54627 | | EST singleton (not in UniGene) with exon | 2.26 | DU145, CALU6, PC3 |
| 335421 | | | CH22_FGENES.55_1 | 2.26 | NCI-H69, PRSC_con, PRSC_log |
| 305260 | AA679280 | Hs.156110 | Immunoglobulin kappa variable 10-8 | 2.26 | NCI-H345, NCI-H69, PRSC_con |
| 303592 | AA421129 | | EST | 2.26 | CALU6, OVCA-R, DU145 |
| 317982 | AI004985 | Hs.130607 | ESTs | 2.26 | PC3, MB-MDA-435s, A549 |
| 325304 | | | CH11_hs gi\|5866910 | 2.26 | MCF7, CALU6, A549 |
| 334118 | | | CH22_FGENES.330_19 | 2.26 | PRSC_con, NCI-H69, PRSC_log |
| 335687 | | | CH22_FGENES.596_2 | 2.26 | A549, CALU6, LnCap |
| 334035 | | | CH22_FGENES.322.3 | 2.26 | NCI-H345, PRSC_con, RPWE-2 |
| 305454 | AA738413 | | EST singleton (not in UniGene) with exon | 2.25 | EB, HT29, CALU6 |
| 335902 | | | CH22_FGENES.635_10 | 2.25 | EB, DU145, HT29 |
| 339215 | | | CH22_FF113D11.GENSCAN.6-10 | 2.25 | PRSC_con, PRSC_log, RPWE-2 |
| 328810 | | | CH.07_hs gi\|5868327 | 2.25 | PC3, OVCA-R, MB-MDA-453 |
| 337396 | | | CH22_FGENES.749-1 | 2.25 | EB, A549, DU145 |
| 336808 | | | CH22_FGENES.205-3 | 2.25 | NCI-H345, NCI-H69, PRSC_con |
| 305808 | AA853958 | | EST singleton (not in UniGene) with exon | 2.24 | MB-MDA-453, DU145, EB |
| 333571 | | | CH22_FGENES.188_2 | 2.24 | MCF7, MB-MDA-453, PC3 |
| 323023 | AA225188 | Hs.258539 | ESTs | 2.24 | EB, DU145, CALU6 |
| 334626 | | | CH22_FGENES.416_2 | 2.24 | NCI-H69, NCI-H345, PRSC_log |
| 333593 | | | CH22_FGENES.210_2 | 2.24 | NCI-H69, NCI-H345, PRSC_con |
| 326708 | | | CH.20_hs gi\|5867593 | 2.24 | NCI-H460, NCI-H23, NCI-H520 |
| 314502 | AI041717 | Hs.132141 | ESTs | 2.23 | NCI-H345, RPWE-2, PRSC_con |
| 309181 | AI951727 | | EST singleton (not in UniGene) with exon | 2.23 | PRSC_con, PC3, MB-MDA-231 |
| 324926 | H56196 | Hs.117798 | ESTs | 2.23 | EB, EB, DU145 |
| 333632 | | | CH22_FGENES.227_3 | 2.23 | CALU6, CALU6, MB-MDA-453 |
| 328243 | | | CH.06_hs gi\|6056292 | 2.23 | PC3, LnCap, LnCap |
| 327037 | | | CH.21_hs gi\|6531965 | 2.23 | LnCap, DU145, EB |
| 307380 | AI222985 | | EST singleton (not in UniGene) with exon | 2.23 | NCI-H345, PRSC_con, PRSC_log |
| 334766 | | | CH22_FGENES.428_15 | 2.23 | PRSC_log, NCI-H345, RPWE-2 |
| 335236 | | | CH22_FGENES.515_8 | 2.23 | OVCA-R, MCF7, BT474 |
| 336615 | | | CH22_FGENES.613_5 | 2.23 | NCI-H69, PRSC_log, PRSC_con |
| 307558 | AI281998 | | EST singleton (not in UniGene) with exon | 2.23 | DU145, OVCA-R, CALU6 |
| 308029 | AI457115 | Hs.62954 | ferritin; heavy polypeptide 1 | 2.23 | EB, OVCA-R, MB-MDA-453 |
| 331508 | N47559 | Hs.46732 | EST | 2.23 | MB-MDA-453, MCF7, BT474 |
| 320980 | AJ237672 | Hs.214142 | 5;10-methylenetetrahydrafolate reductase | 2.23 | OVCA-R, EB, EB |
| 304241 | AA010976 | | EST singleton (not in UniGene) with exon | 2.23 | BT474, MB-MDA-435s, MB-MDA-231 |
| 314682 | AI190864 | Hs.178226 | ESTs; Weakly similar to !!!! ALU SUBFAMI | 2.23 | MB-MDA-231, MCF7, OVCA-R |
| 308382 | AI624301 | | EST singleton (not in UniGene) with exon | 2.22 | OVCA-R, BT474, CALU6 |
| 314476 | AW207857 | Hs.169604 | ESTs | 2.22 | DU145, EB, A549 |
| 327864 | | | CH.06_hs gi\|5868130 | 2.22 | NCI-H69, PRSC_log, PRSC_con |
| 337279 | | | CH22_FGENES.665-2 | 2.22 | NCI-H345, NCI-H69, PRSC_con |
| 302263 | AA325517 | | EST | 2.22 | BT474, NCI-H520, DU145 |
| 322840 | AA083710 | | EST cluster (not in UniGene) | 2.22 | HT29, MB-MDA-453, CALU6 |
| 307574 | AI283549 | | EST singleton (not in UniGene) with exon | 2.22 | OVCA-R, CALU6, BT474 |
| 319027 | AA716812 | | EST cluster (not in UniGene) | 2.22 | LnCap, NCI-H69, NCI-H69 |
| 305925 | AA877883 | | EST singleton (not in UniGene) with exon | 2.22 | NCI-H345, NCI-H69, NCI-H69 |
| 329725 | | | CH.14_p2 gi\|6065785 | 2.22 | NCI-H69, PRSC_con, NCI-H345 |
| 316194 | AW298529 | Hs.255774 | ESTs | 2.22 | CALU6, EB, NCI-H520 |
| 301119 | AF142579 | | EST | 2.22 | A549, OVCA-R, EB |
| 333815 | | | CH22_FGENES.282_4 | 2.22 | MB-MDA-435s, EB, MB-MDA-453 |
| 334358 | | | CH22_FGENES.378_1 | 2.22 | NCI-H345, RPWE-2, PRSC_con |
| 303763 | AF043250 | Hs.30928 | DNA segment on chromosome 19 (unique) 11 | 2.21 | Caco2, NCI-H23, NCI-H520 |
| 335593 | | | CH22_FGENES.581_32 | 2.21 | NCI-H345, PRSC_log, RPWE-2 |
| 334026 | | | CH22_FGENES.318_3 | 2.21 | NCI-H69, PRSC_con, NCI-H345 |
| 322224 | AF086064 | | EST cluster (not in UniGene) | 2.21 | PRSC_con, PRSC_log, RPWE-2 |
| 309836 | AW295497 | Hs.157397 | ESTs | 2.21 | NCI-H345, PRSC_con, RPWE-2 |
| 332669 | M33374 | Hs.661 | NADH dehydrogenase (ubiquinone) 1 betas | 2.21 | NCI-H520, CALU6, OVCA-R |
| 307629 | AI300246 | | EST singleton (not in UniGene) with exon | 2.21 | MB-MDA-231, MB-MDA-453, HT29 |
| 300470 | T87841 | | EST | 2.21 | PC3, EB, CALU6 |
| 330084 | | | CH.19_p2 gi\|6165044 | 2.21 | NCI-H69, PRSC_con, BT474 |
| 338819 | | | CH22_DJ246D7.GENSCAN.1-24 | 2.21 | NCI-H69, RPWE-2, PRSC_log |
| 337797 | | | CH22_EM:AC005500.GENSCAN.3-4 | 2.21 | LnCap, NCI-H69, NCI-H520 |
| 328025 | | | CH.06_hs gi\|5902482 | 2.2 | RPWE-2, PRSC_con, PRSC_log |
| 326240 | | | CH.17_hs gi\|5867260 | 2.2 | EB, LnCap, MB-MDA-453 |
| 312865 | AW005376 | Hs.173280 | ESTs | 2.2 | DU145, DU145, OVCA-R |
| 338450 | | | CH22_EM:AC005500.GENSCAN.359-36 | 2.2 | MCF7, MB-MDA-453, MB-MDA-435s |
| 302532 | U60181 | Hs.248115 | growth hormone secretagogue receptor | 2.2 | PRSC_con, PRSC_log, PRSC_log |
| 321132 | AA081495 | | EST cluster (not in UniGene) | 2.2 | NCI-H23, NCI-H520, NCI-358 |
| 337787 | | | CH22_EM:AC000097.GENSCAN.123-3 | 2.2 | EB, PC3, LnCap |
| 337032 | | | CH22_FGENES.438-3 | 2.2 | NCI-H69, NCI-H345, RPWE-2 |
| 300026 | M11507 | | AFFX control: transferrin receptor | 2.2 | HT29, EB, MB-MDA-231 |
| 333139 | | | CH22_FGENES.83_16 | 2.2 | HT29, MB-MDA-453, Caco2 |
| 334298 | | | CH22_FGENES.372_4 | 2.2 | PRSC_con, PRSC_log, RPWE-2 |
| 335002 | | | CH22_FGENES.470_7 | 2.2 | PRSC_con, NCI-H345, NCI-H345 |
| 335000 | | | CH22_FGENES.470_5 | 2.2 | EB, PC3, A549 |
| 337298 | | | CH22_FGENES.678-3 | 2.2 | NCI-H69, A549, HT29 |
| 302481 | AF104253 | Hs.241381 | cofactor required for Sp1 transcriptiona | 2.2 | EB, CALU6, LnCap |
| 334819 | | | CH22_FGENES.436_15 | 2.19 | CALU6, BT474, Caco2 |
| 300426 | AW452660 | Hs.253296 | ESTs | 2.19 | DU145, CALU6, HT29 |
| 302569 | AC004472 | | multiple UniGene matches | 2.19 | RPWE-2, PRSC_log, PRSC_con |
| 339401 | | | CH22_BA232E17.GENSCAN.7-7 | 2.19 | NCI-H345, NCI-H69, PRSC_log |
| 328791 | | | CH.07_hs gi\|5868309 | 2.19 | DU145, PC3, HT29 |
| 337333 | | | CH22_FGENES.711-3 | 2.19 | NCI-H69, NCI-H345, PRSC_log |
| 339363 | | | CH22_BA354I12.GENSCAN.33-6 | 2.19 | NCI-H69, PRSC_log, PRSC_con |
| 329429 | | | CH.Y_hs gi\|5868882 | 2.19 | CALU6, HT29, OVCA-R |
| 336927 | | | CH22_FGENES.348-3 | 2.19 | NCI-869, PRSC_log, NCI-358 |
| 336351 | | | CH22_FGENES.816_3 | 2.19 | DU145, EB, MB-MDA-231 |
| 313466 | AA004731 | Hs.148876 | ESTs | 2.19 | CALU6, DU145, OVCA-R |
| 307433 | AI244895 | | EST singleton (not in UniGene) with exon | 2.19 | NCI-H23, NCI-H23, NCI-358 |
| 336590 | | | CH22_FGENES.51_2 | 2.19 | PRSC_con, NCI-H69, PRSC_log |
| 310758 | AI770001 | Hs..209445 | ESTs | 2.18 | EB, MB-MDA-231, BT474 |
| 327823 | | | CH.05_hs gi\|5867968 | 2.18 | PRSC_con, NCI-H69, NCI-H345 |
| 313257 | N92638 | | EST cluster (not in UniGene) | 2.18 | PRSC_log, RPWE-2, PRSC_con |
| 335377 | | | CH22_FGENES.543_17 | 2.18 | PC3, MB-MDA-435s, CALU6 |
| 303958 | AL042931 | | EST singleton (not in UniGene) with exon | 2.18 | NCI-H345, RPWE-2, PRSC_con |
| 320153 | AF064594 | Hs.120360 | phospholipase A2; group VI | 2.18 | LnCap, PC3, MB-MDA-435s |
| 335201 | | | CH22_FGENES.508_10 | 2.18 | OVCA-R, DU145, HT29 |
| 338591 | | | CH22.EMAC005500.GENSCAN.434-4 | 2.18 | NCI-H69, NCI-H345, RPWE-2 |
| 331958 | AA455960 | Hs.99405 | ESTs | 2.18 | MCF7, NCI-H23, NCI-H460 |
| 337218 | | | CH22_FGENES.614-2 | 2.18 | CALU6, A549, MCF7 |
| 309470 | AW118833 | | EST singleton (not in UniGene) with exon | 2.18 | PC3, EB, MB-MDA-435s |
| 331896 | AA435495 | Hs.97174 | H sapiens mRNA; cDNA DKFZp566E164 (from | 2.18 | RPWE-2, NCI-H69, PRSC_log |
| 330275 | | | CH.05_p2 gi\|6671904 | 2.18 | NCI-H345, PRSC_log, PRSC_con |
| 335817 | | | CH22_FGENES.618_5 | 2.18 | A549, Caco2, PC3 |
| 332896 | | | CH22_FGENES.35_10 | 2.18 | NCI-H345, RPWE-2, PRSC_log |
| 303294 | AA205300 | | EST | 2.17 | MB-MDA-435s, A549, MCF7 |
| 338703 | | | CH22_EM:AC005500.GENSCAN.480-2 | 2.17 | HT29, BT474, NCI-H69 |
| 300115 | AI215044 | Hs.208130 | ESTs | 2.17 | PC3, OVCA-R, HT29 |
| 330979 | H22466 | Hs.31795 | ESTs | 2.17 | MCF7, EB, MB-MDA-435s |
| 317246 | AW105092 | Hs.155690 | ESTs | 2.17 | MB-MDA-453, DU145, EB |
| 329078 | | | CH.X_hs gi\|5868597 | 2.17 | MB-MDA-453, MB-MDA-231, BT474 |
| 312554 | AI222630 | Hs.109390 | ESTs | 2.17 | NCI-H520, OVCA-R, MCF7 |
| 323207 | AI052795 | Hs.192201 | ESTs | 2.17 | NCI-H69, NCI-H345, PRSC_log |
| 301894 | AA484435 | Hs.41997 | alpha-1-B glycoprotein | 2.17 | PRSC_con, LnCap, PRSC_log |
| 329097 | | | CH.X_hs gi\|5868624 | 2.16 | MB-MDA-231, MCF7, NCI-358 |
| 328328 | | | CH.07_hs gi\|5868375 | 2.16 | NCI-H345, PRSC_con, NCI-H69 |
| 302671 | AA522440 | Hs.135917 | ESTs | 2.16 | BT474, DU145, A549 |
| 329201 | | | CH.X_hs gi\|5868718 | 2.16 | OVCA-R, PC3, MB-MDA-435s |
| 329902 | | | CH.15_p2 gi\|6634760 | 2.16 | PRSC_con, NCI-H69, NCI-H345 |
| 334435 | | | CH22_FGENES.385_10 | 2.16 | PRSC_con, NCI-H345, RPWE-2 |
| 330742 | AA400979 | Hs.25691 | calcitonin receptor-like receptor activi | 2.16 | MCF7, MB-MDA-453, PC3 |
| 328484 | | | CH.07_hs gi\|5868454 | 2.16 | NCI-H69, PRSC_log, NCI-H345 |
| 334784 | | | CH22_FGENES.432_9 | 2.16 | PRSC_log, RPWE-2, PRSC_con |
| 337771 | | | CH22.EM:AC000097.GENSCAN.119-10 | 2.16 | NCI-H69, PRSC_con, RPWE-2 |
| 300181 | AI284955 | Hs.157568 | ESTs; Weakly similar to ataxin-2 [M.musc | 2.16 | DU145, EB, CALU6 |
| 300268 | AI539446 | Hs.245450 | ESTs | 2.16 | PRSC_con, RPWE-2, PRSC_log |
| 309575 | AW168096 | Hs.195188 | glyceraldehyde-3-phosphate dehydrogenase | 2.16 | A549, NCI-H23, MB-MDA-453 |
| 336548 | | | CH22_FGENES.841_5 | 2.16 | NCI-H345, NCI-H69, MB-MDA-231 |
| 328506 | | | CH.07_hs gi\|5868471 | 2.16 | EB, A549, CALU6 |
| 330189 | | | CH.05_p2 gi\|6165182 | 2.16 | NCI-H460, MCF7, MB-MDA-453 |
| 305480 | AA746500 | Hs.25911 | HLA-B associated transcript-2 | 2.16 | EB, DU145, NCI-358 |
| 302270 | R56151 | | EST | 2.16 | OVCA-R, MB-MDA-435s, PRSC_con |
| 306669 | AI004899 | | EST singleton (not in UniGene) with exon | 2.16 | PRSC_log, PRSC_con, NCI-H345 |
| 325887 | | | CH.16_hs gi\|5867087 | 2.16 | EB, CALU6, NCI-358 |
| 327015 | | | CH.21_hs gi\|5867664 | 2.15 | EB, PC3, HT29 |
| 338576 | | | CH22_EM:AC005500.GENSCAN.429-1 | 2.15 | NCI-H69, NCI-H345, PRSC_con |
| 333592 | | | CH22_FGENES.209_2 | 2.15 | NCI-H69, OVCA-R, PRSC_con |
| 317253 | AW071241 | Hs.199685 | ESTs | 2.15 | MB-MDA-435s, NCI-H23, MB-MDA-453 |
| 302301 | R67493 | Hs.127150 | ESTs; Weakly similar to ZINC FINGER PROT | 2.15 | PC3, MCF7, MB-MDA-435s |
| 336858 | | | CH22_FGENES.293-8 | 2.15 | RPWE-2, PRSC_con, NCI-H69 |
| 308417 | AI640693 | Hs.2186 | eukaryotic translation elongation factor | 2.15 | EB, OVCA-R, CALU6 |
| 338177 | | | CH22_EM:AC005500.GENSCAN.219-5 | 2.15 | NCI-H345, NCI-H23, NCI-H520 |
| 337592 | | | CH22_C20H12.GENSCAN.6-7 | 2.15 | PC3, A549, HT29 |
| 325945 | | | CH.16_hs gi\|5867138 | 2.15 | MB-MDA-453, MB-MDA-435s, DU145 |
| 335262 | | | CH22_FGENES.520_3 | 2.15 | EB, PC3, A549 |
| 333665 | | | CH22_FGENES.244_1 | 2.15 | PRSC_con, RPWE-2, PRSC_log |
| 333710 | | | CH22_FGENES.250_25 | 2.14 | PRSC_log, NCI-H69, PRSC_con |
| 304927 | AA604728 | Hs.195188 | glyceraldehyde-3-phosphate dehydrogenase | 2.14 | LnCap, PC3, MCF7 |
| 336999 | | | CH22_FGENES.417-20 | 2.14 | NCI-H69, NCI-H345, PRSC_con |
| 313283 | W32480 | Hs.157099 | ESTs | 2.14 | EB, MB-MDA-231, A549 |
| 306221 | AA928686 | | EST singleton (not in UniGene) with exon | 2.14 | NCI-H460, PRSC_con, NCI-H23 |
| 333205 | | | CH22_FGENES.102_5 | 2.14 | NCI-H69, PRSC_con, PRSC_log |
| 312932 | AI804218 | Hs.209614 | ESTs | 2.14 | PRSC_con, NCI-H345, RPWE-2 |
| 328938 | | | CH.08_hs gi\|5868500 | 2.14 | HT29, PC3, MB-MDA-453 |
| 326748 | | | CH.20_hs gi\|5867611 | 2.14 | NCI-H345, NCI-H69, PRSC_con |
| 337964 | | | CH22_EM:AC005500.GENSCAN.100-9 | 2.14 | RPWE-2, PRSC_con, PRSC_log |
| 337984 | | | CH22_EM:AC005500.GENSCAN.110-2 | 2.14 | EB, DU145, NCI-H345 |
| 337704 | | | CH22_EM.AC000097.GENSCAN.87-6 | 2.14 | NCI-H69, NCI-H460, NCI-358 |
| 302162 | AF119046 | | EST | 2.14 | MB-MDA-435s, PC3, EB |
| 303192 | AA081755 | Hs.8059 | ESTs; Highly similar to SYNAPTOTAGMIN IV | 2.14 | MB-MDA-435s, MB-MDA-435s, MB-MDA-453 |
| 306200 | AA926816 | | EST singleton (not in UniGene) with exon | 2.14 | MB-MDA-453, CALU6, DU145 |
| 303996 | AW515979 | Hs.84298 | CD74 antigen (invanant polypptd of majo | 2.14 | LnCap, MB-MDA-231, BT474 |
| 325409 | | | CH.12_hs gi\|5866921 | 2.14 | PRSC_log, PRSC_con, RPWE-2 |
| 308558 | AI700145 | Hs.172182 | poly(A)-binding protein; cytoplasmic 1 | 2.14 | MCF7, ER, MB-MDA-435s |
| 302185 | AA243837 | Hs.156915 | ESTs | 2.14 | MB-MDA-453, MCF7, EB |
| 303021 | W39612 | | EST | 2.14 | PRSC_con, NCI-H69, RPWE-2 |
| 301005 | AW451916 | Hs.210848 | ESTs | 2.14 | DU145, EB, HT29 |
| 336029 | | | CH22_FGENES.672_4 | 2.14 | NCI-H69, PRSC_con, RPWE-2 |
| 305443 | AA736653 | | EST singleton (not in UniGene) with exon | 2.14 | NCI-358, NCI-H520, NCI-H23 |
| 335485 | | | CH22_FGENES.570_17 | 2.13 | NCI-H460, MB-MDA-435s, MCF7 |
| 304817 | AA584712 | | EST singleton (not in UniGene) with exon | 2.13 | MCF7, MCF7, NCI-H520 |
| 309859 | AW298760 | | EST singleton (not in UniGene) with exon | 2.13 | NCI-H69, PRSC_con, LnCap |
| 326206 | | | CH.17_hs gi\|5867219 | 2.13 | EB, MB-MDA-231, LnCap |
| 303656 | AA437189 | Hs.122574 | ESTs | 2.13 | LnCap, MB-MDA-435s, EB |
| 334745 | | | CH22_FGENES.426_3 | 2.13 | OVCA-R, DU145, MB-MDA-453 |
| 318504 | T26453 | | EST cluster (not in UniGene) | 2.13 | RPWE-2, LnCap, CALU6 |
| 306839 | AI077385 | | EST singleton (not in UniGene) with exon | 2.13 | MCF7, MB-MDA-453, MB-MDA-435s |
| 303843 | W94322 | Hs.58094 | melanoma inhibitory activity | 2.13 | MB-MDA-435s, NCI-H345, RPWE-2 |
| 308444 | AI659398 | Hs.197097 | EST | 2.13 | MB-MDA-453, MCF7, BT474 |
| 301322 | AW448965 | Hs.256305 | ESTs | 2.13 | NCI-H345, LnCap, PC3 |
| 326997 | | | CH.21_hs gi\|5867660 | 2.13 | HT29, A549, CALU6 |
| 326793 | | | CH.20_hs gi\|5867631 | 2.13 | PRSC_log, PRSC_con, MB-MDA-453 |
| 320360 | H12405 | | EST cluster (not in UniGene) | 2.12 | MB-MDA-231, BT474, HT29 |
| 316301 | AW206279 | Hs.192009 | ESTs | 2.12 | DU145, DU145, EB |
| 335371 | | | CH22_FGENES.543_9 | 2.12 | PC3, MB-MDA-435s, DU145 |
| 301178 | AA828385 | | EST | 2.12 | EB, OVCA-R, LnCap |
| 326136 | | | CH.17_hs gi\|5867202 | 2.12 | RPWE-2, PRSC_log, PRSC_con |
| 339213 | | | CH22_FF113D11.GENSCAN.6.8 | 2.12 | OVCA-R, PC3, MB-MDA-231 |
| 335980 | | | CH22_FGENES.653_2 | 2.12 | BT474, BT474, OVCA-R |
| 314380 | AA758797 | Hs.192807 | ESTs | 2.11 | PRSC_con, PRSC_log, RPWE-2 |
| 306779 | AI041302 | | EST singleton (not in UniGene) with exon | 2.11 | NCI-H345, PRSC_con, PRSC_log |
| 335774 | | | CH22_FGENES.607_10 | 2.11 | PC3, A549, MB-MDA-453 |
| 334914 | | | CH22_FGENES.457_3 | 2.11 | PRSC_con, NCI-H345, NCI-H69 |
| 304619 | AA515554 | Hs.119598 | ribosomal protein L3 | 2.11 | EB, MB-MDA-453, MB-MDA-435s |
| 303358 | AI199714 | Hs.158149 | ESTs | 2.11 | CALU6, OVCA-R, DU145 |
| 306558 | AA994743 | | EST singleton (not in UniGene) with exon | 2.11 | HT29, MB-MDA-453, CALU6 |
| 337781 | | | CH22_EM:AC000097.GENSCAN.121-3 | 2.11 | PRSC_log, PRSC_con, RPWE-2 |
| 333140 | | | CH22_FGENES.84_1 | 2.11 | HT29, NCI-H69, OVCA-R |
| 315081 | AI247134 | Hs.155281 | ESTs | 2.11 | MB-MDA-453, MCF7, HT29 |
| 302965 | AA446441 | Hs.138842 | ESTs | 2.11 | NCI-358, NCI-H23, CALU6 |
| 302138 | N83965 | | EST | 2.11 | PRSC_log, PRSC_con, NCI-H345 |
| 320802 | D83824 | Hs.185055 | BENE protein | 2.11 | A549, PC3, HT29 |
| 322152 | AA565332 | | EST cluster (not in UniGene) | 2.11 | A549, CALU6, EB |
| 326418 | | | CH.19_hs gi\|5867365 | 2.1 | EB, OVCA-R, DU145 |
| 308709 | AI783498 | Hs.181165 | eukaryotic translation elongation factor | 2.1 | MB-MDA-435s, MB-MDA-453, DU145 |
| 332737 | C01852 | Hs.84359 | hypothetical protein | 2.1 | NCI-H23, A549, DU145 |
| 333283 | | | CH22_FGENES.128_13 | 2.1 | NCI-H345, RPWE-2, PRSC_con |
| 328636 | | | CH.07_hs gi\|6004473 | 2.1 | DU145, EB, MB-MDA-453 |
| 329187 | | | CH.X_hs gi\|5868713 | 2.1 | NCI-358, NCI-H23, NCI-H460 |
| 305999 | AA889603 | | EST singleton (not in UniGene) with exon | 2.1 | HT29, OVCA-R, PC3 |
| 333220 | | | CH22_FGENES.104_12 | 2.1 | PRSC_con, PRSC_log, RPWE-2 |
| 335092 | | | CH22_FGENES.492_2 | 2.1 | NCI-H69, PRSC_con, NCI-H345 |
| 304887 | AA599355 | | EST singleton (not in UniGene) with exon | 2.1 | DU145, EB, MCF7 |
| 325359 | | | CH.12_hs gi\|5866920 | 2.1 | MB-MDA-453, EB, MB-MDA-435s |
| 330956 | H08730 | Hs.6933 | ESTs | 2.1 | NCI-H520, PRSC_con, NCI-H345 |
| 323786 | AW449315 | Hs.165795 | ESTs | 2.1 | OVCA-R, A549, LnCap |
| 333619 | | | CH22_FGENES.219_3 | 2.1 | BT474, OVCA-R, HT29 |
| 324538 | AW502979 | | EST cluster (not in UniGene) | 2.09 | CALU6, A549, DU145 |
| 303405 | AA308601 | | EST | 2.09 | DU145, CALU6, NCI-H69 |
| 328570 | | | CH.07_hs gi\|5868231 | 2.09 | LnCap, MB-MDA-231, DU145 |
| 308971 | AI871218 | Hs.224731 | EST | 2.09 | NCI-H23, NCI-H460, NCI-358 |
| 330467 | K02268 | Hs.22584 | prodynorphin | 2.09 | PC3, BT474, MB-MDA-453 |
| 334793 | | | CH22_FGENES.433_5 | 2.09 | EB, DU145, LnCap |
| 300908 | AA618335 | Hs.146137 | ESTs; Weakly similar to putative [C.eleg | 2.09 | NCI-H345, PRSC_log, PRSC_con |
| 309656 | AW197060 | Hs.195188 | glyceraldehyde-3-phosphate dehydrogenase | 2.09 | A549, NCI-H23, NCI-H460 |
| 320963 | AB029041 | Hs.209646 | KIAA1118 protein | 2.09 | PRSC_con, PRSC_log, NCI-H345 |
| 310833 | AW295351 | Hs.169136 | ESTs | 2.09 | PC3, LnCap, MB-MDA-453 |
| 335693 | | | CH22_FGENES.596_8 | 2.09 | NCI-H69, LnCap, PRSC_log |
| 325966 | | | CH.16_hs gi\|5867147 | 2.09 | MCF7, CALU6, MB-MDA-453 |
| 329319 | | | CH.X_hs gi\|6381976 | 2.09 | NCI-H460, EB, DU145 |
| 338526 | | | CH22_EM:AC005500.GENSCAN.396-14 | 2.09 | NCI-H69, NCI-H345, PRSC_log |
| 336751 | | | CH22_FGENES.128-5 | 2.09 | NCI-H69, NCI-H345, PRSC_log |
| 325510 | | | CH.12_hs gi\|5866974 | 2.09 | HT29, OVCA-R, CALU6 |
| 323553 | AA292626 | Hs.122854 | ESTs | 2.08 | NCI-H345, RPWE-2, NCI-358 |
| 326343 | | | CH.17_hs gi\|8525295 | 2.08 | EB, LnCap, DU145 |
| 335470 | | | CH22_FGENES.568_3 | 2.08 | NCI-H69, PRSC_con, PRSC_log |
| 320122 | T93681 | Hs.187515 | ESTs | 2.08 | MCF7, MB-MDA-453, BT474 |
| 335320 | | | CH22_FGENES.534_7 | 2.08 | BT474, MB-MDA-231, HT29 |
| 307120 | AI184343 | | EST singleton (not in UniGene) with exon | 2.08 | HT29, MCF7, PC3 |
| 338080 | | | CH22_EM:AC005500.GENSCAN.172-11 | 2.08 | LnCap, PC3, HT29 |
| 313113 | AI056258 | Hs.122523 | ESTs | 2.08 | MCF7, DU145, MB-MDA-453 |
| 337685 | | | CH22_EM:AC000097.GENSCAN.77-1 | 2.08 | NCI-H69, NCI-H345, PRSC_log |
| 327461 | | | CH.02_hs gi\|6004455 | 2.08 | NCI-H23, BT474, NCI-358 |
| 335895 | | | CH22_FGENES.635_3 | 2.08 | HT29, MB-MDA-231, NCI-H520 |
| 303933 | AW471472 | | EST singleton (not in UniGene) with exon | 2.08 | MS-MDA-231, BT474, NCI-H345 |
| 314803 | AI935159 | Hs.166841 | ESTs; Weakly similar to MYOSIN LIGHT CHA | 2.08 | PC3, A549, BT474 |
| | | | NON-MUSCLE ISOZYMES [H.sapiens] | | |
| 302722 | U53530 | | EST | 2.08 | DU145, MB-MDA-435s, OVCA-R |
| 307703 | AI318588 | | EST singleton (not in UniGene) with exon | 2.08 | HT29, MB-MDA-435s, CALU6 |
| 310558 | AI334965 | Hs.176976 | ESTs | 2.08 | A549, LnCap, PC3 |
| 315276 | AA860090 | | EST cluster (not in UniGene) | 2.08 | PC3, MCF7, OVCA-R |
| 306443 | AA976950 | | EST singleton (not in UniGene) with exon | 2.07 | OVCA-R, PC3, EB |
| 307961 | AI421059 | | EST singleton (not in UniGene) with exon | 2.07 | HT29, OVCA-R, CALU6 |
| 329735 | | | CH.14_p2 gi\|6065780 | 2.07 | EB, HT29, OVCA-R |
| 335193 | | | CH22_FGENES.507_8 | 2.07 | EB, A549, A549 |
| 320347 | R34423 | Hs.221535 | ESTs | 2.07 | CALU6, A549, EB |
| 316153 | AA724474 | Hs.147208 | ESTs | 2.07 | MS-MDA-453, PC3, HT29 |
| 300921 | AW293224 | Hs.232165 | ESTs | 2.07 | HT29, CALU6, CALU6 |
| 319264 | T65096 | | EST cluster (not in UniGene) | 2.07 | MS-MDA-453, MCF7, CALU6 |
| 330204 | | | CH.05_p2 gi\|6013606 | 2.07 | OVCA-R, DU145, EB |
| 317070 | AI142037 | Hs.125379 | ESTs | 2.07 | PRSC_con, NCI-H345, OVCA-R |
| 337645 | | | CH22.EM:AC000097.GENSCAN.10-8 | 2.07 | NCI-H345, PRSC_log, NCI-H69 |
| 312501 | AW450490 | Hs.132886 | ESTs | 2.07 | NCI-H520, CALU6, MCF7 |
| 335587 | | | CH22_FGENES.581_26 | 2.07 | NCI-H69, NCI-H345, PRSC_log |
| 311482 | AI917706 | Hs.129997 | ESTs | 2.07 | NCI-H520, MCF7, MS-MDA-435s |
| 302488 | AF161441 | | EST | 2.07 | EB, DU145, CALU6 |
| 304692 | AA554202 | Hs.76067 | heat shock 27 kD protein 1 | 2.07 | MCF7, MB-MDA-453, PC3 |
| 325369 | | | CH.12_hs gi\|5866920 | 2.07 | DU145, DU145, MS-MDA-453 |
| 306284 | AA936835 | | EST singleton (not in UniGene) with exon | 2.07 | BT474, MB-MDA-231, HT29 |
| 337402 | | | CH22_FGENES.752-1 | 2.07 | A549, BT474, DU145 |
| 327418 | | | CH.02_hs gi\|5867750 | 2.07 | MCF7, MB-MDA-453, MB-MDA-435s |
| 317977 | AI004775 | Hs.205091 | ESTs; Weakly similar to WW domain bindin | 2.07 | BT474, MB-MDA-453, PC3 |
| 331870 | AA428560 | Hs.161845 | EST | 2.07 | MB-MDA-231, MB-MDA-435s, BT474 |
| 300750 | AA514805 | Hs.105464 | ESTs | 2.07 | HT29, BT474, BT474 |
| 336657 | | | CH22_FGENES.35-14 | 2.07 | MB-MDA-453, MCF7, NCI-H460 |
| 336035 | | | CH22_FGENES.678_6 | 2.07 | NCI-H69, PRSC_con, RPWE-2 |
| 325320 | | | CH.11_hs gi\|5866870 | 2.06 | NCI-H69, PRSC_log, PRSC_con |
| 306053 | AA905312 | | EST singleton (not in UniGene) with exon | 2.06 | HT29, OVCA-R, MB-MDA-231 |
| 333175 | | | CH22_FGENES.95_2 | 2.06 | LnCap, HT29, DU145 |
| 304491 | AA437096 | Hs.115502 | EST | 2.06 | MB-MDA-435s, CALU6, CALU6 |
| 310632 | AI697536 | Hs.176991 | ESTs | 2.06 | NCI-H69, PRSC_log, NCI-H345 |
| 338521 | | | CH22_EMAC005500.GENSCAN.395-35 | 2.06 | NCI-H345, PRSC_log, PRSC_log |
| 334900 | | | CH22_FGENES.452_14 | 2.06 | A549, CALU6, NCI-H69 |
| 337451 | | | CH22_FGENES.774-2 | 2.06 | PRSC_con, PRSC_log, RPWE-2 |
| 306792 | AI815153 | Hs.195188 | glyceraldehyde-3-phosphate dehydrogenase | 2.06 | DU145, BT474, MB-MDA-453 |
| 336854 | | | CH22_FGENES.280-1 | 2.06 | LnCap, EB, MB-MDA-435s |
| 304485 | AA434076 | | EST singleton (not in UniGene) with exon | 2.06 | MB-MDA-231, BT474, CALU6 |
| 326458 | | | CH.19_hs gi\|5867400 | 2.06 | EB, DU145, LnCap |
| 303506 | AA340605 | Hs.105887 | ESTs | 2.06 | LnCap, MCF7, CALU6 |
| 333628 | | | CH22_FGENES.226_2 | 2.06 | NCI-H520, NCI-358, NCI-358 |
| 300763 | AA190753 | | EST | 2.06 | NCI-H69, NCI-H345, PRSC_con |
| 334836 | | | CH22_FGENES.439_6 | 2.06 | NCI-H345, PRSC_con, RPWE-2 |
| 335217 | | | CH22_FGENES.512_3 | 2.06 | PRSC_log, PRSC_con, NCI-H69 |
| 338970 | | | CH22_DJ32I10.GENSCAN.26-3 | 2.06 | A549, MB-MDA-453, LnCap |
| 334842 | | | CH22_FGENES.439_21 | 2.06 | DU145, HT29, CALU6 |
| 309309 | AW006428 | Hs.232857 | EST | 2.06 | EB, DU145, OVCA-R |
| 332949 | | | CH22_FGENES.47_12 | 2.06 | EB, DU145, OVCA-R |
| 310530 | AW369663 | Hs.150150 | ESTs | 2.06 | PRSC_con, PRSC_log, RPWE-2 |
| 329401 | | | CH.X_hs gi\|6682544 | 2.06 | NCI-H69, PRSC_con, RPWE-2 |
| 316893 | AA837332 | | EST duster (not in UniGene) | 2.06 | OVCA-R, MCF7, MR-MDA-453 |
| 325022 | W95840 | Hs.59745 | NADH dehydrogenase (ubiquinone) flavopro | 2.06 | Caco2, NCI-358, OVCA-R |
| 329839 | | | CH.14_p2 gi\|6672062 | 2.05 | MB-MDA-231, RPWE-2, CALU6 |
| 306668 | AI004890 | | EST singleton (not in UniGene) with exon | 2.05 | DU145, MB-MDA-453, MCF7 |
| 315604 | AW137442 | Hs.136965 | ESTs | 2.05 | LnCap, EB, PC3 |
| 318551 | AI909951 | Hs.239307 | tyrosyl-tRNA synthetase | 2.05 | NCI-H345, PRSC_con, RPWE-2 |
| 339344 | | | CH22_BA354I12.GENSCAN.28-1 | 2.05 | BT474, MR-MDA-231, A549 |
| 310621 | AI632098 | Hs.198099 | ESTs | 2.05 | NCI-H69, RPWE-2, MCF7 |
| 327051 | | | CH.21_hs gi\|6531965 | 2.05 | PRSC_con, NCI-H345, PRSC_log |
| 336827 | | | CH22_FGENES.236-2 | 2.05 | NCI-H345, A549, MB-MDA-231 |
| 311846 | AI078033 | Hs.177170 | ESTs; Moderately similar to !!!! ALU SUB | 2.05 | OVCA-R, DU145, CALU6 |
| 335036 | | | CH22_FGENES.475_14 | 2.05 | NCI-H69, PRSC_con, NCI-H345 |
| 313100 | N52880 | Hs.122817 | ESTs | 2.05 | RPWE-2, NCI-H345, PRSC_log |
| 301927 | AF014459 | Hs.113250 | retinoschisis (X-linked; juvenile) 1 | 2.05 | MB-MDA-231, NCI-H345, PRSC_con |
| 326070 | | | CH.17_hs gi\|5867175 | 2.05 | MB-MDA-435s, MB-MDA-231, BT474 |
| 338514 | | | CH22_EM:AC005500.GENSCAN.392-4 | 2.05 | PRSC_con, PRSC_log, RPWE-2 |
| 328098 | | | CH.06_hs gi\|5868020 | 2.05 | DU145, CALU6, EB |
| 301102 | AA679361 | Hs.249487 | ESTs | 2.05 | NCI-H460, PRSC_con, NCI-H23 |
| 306193 | AA923457 | | EST singleton (not in UniGene) with exon | 2.05 | NCI-H345, PRSC_con, RPWE-2 |
| 317027 | AA883808 | Hs.174148 | ESTs | 2.05 | EB, DU145, CALU6 |
| 336102 | | | CH22_FGENES.693_2 | 2.04 | LnCap, NCI-H69, PRSC_log |
| 301372 | AI239895 | Hs.130555 | ESTs | 2.04 | PRSC_con, RPWE-2, PRSC_log |
| 333252 | | | CH22_FGENES.116_4 | 2.04 | NCI-358, A549, HT29 |
| 322516 | AW372340 | Hs.159717 | ESTs | 2.04 | HT29, MB-MDA-231, BT474 |
| 324146 | AA393624 | | EST cluster (not in UniGene) | 2.04 | RPWE-2, PRSC_con, MB-MDA-231 |
| 338770 | | | CH22_EM:AC005500.GENSCAN.520-1 | 2.04 | PRSC_con, NCI-H69, NCI-H460 |
| 314795 | AI798611 | Hs.157277 | ESTs | 2.04 | EB, PC3, LnCap |
| 333004 | | | CH22_FGENES.60_1 | 2.04 | A549, NCI-358, DU145 |
| 302405 | AW245825 | Hs.211914 | NADH dehydrogenase (ubiquinone) Fe-S pro | 2.04 | NCI-H520, CALU6, Caco2 |
| 323587 | AI905527 | Hs.141901 | ESTs; Moderately similar to !!!! ALU SUB | 2.04 | EB, A549, HT29 |
| 300898 | AI276278 | Hs.157176 | ESTs | 2.04 | PC3, MR-MDA-453, BT474 |
| 301506 | AI149878 | Hs.143519 | ESTs; Weakly similar to testicular tekti | 2.04 | NCI-H69, RPWE-2, NCI-H345 |
| 325851 | | | CH.16_hs gi\|5867067 | 2.04 | MB-MDA-231, HT29, EB |
| 323945 | AI125604 | Hs.155117 | ESTs | 2.04 | MCF7, DU145, DU145 |
| 303265 | AW160951 | | EST | 2.04 | LnCap, OVCA-R, DU145 |
| 334135 | | | CH22_FGENES.336_2 | 2.04 | PC3, A549, MB-MDA-435s |
| 329793 | | | CH.14_p2 gi\|8522661 | 2.04 | DU145, CALU6, HT29 |
| 332595 | AA256431 | Hs.3244 | G protein pathway suppressor 2 | 2.04 | A549, CALU6, NCI-H23 |
| 316059 | AW166388 | Hs.250181 | ESTs | 2.04 | MCF7, HT29, A549 |
| 324104 | AW248071 | Hs.133122 | ESTs | 2.04 | Caco2, A549, MCF7 |
| 306801 | AI052653 | | EST singleton (not in UniGene) with exon | 2.03 | EB, LnCap, PC3 |
| 338096 | | | CH22.EM:AC005500.GENSCAN.181-14 | 2.03 | DU145, HT29, CALU6 |
| 327544 | | | CH.03_hs gi\|5867797 | 2.03 | PRSC_con, NCI-H69, NCI-H345 |
| 318813 | F13195 | | EST cluster (not in UniGene) | 2.03 | PRSC_con, RPWE-2, PRSC_log |
| 325289 | | | CH.11_hs gi\|5866903 | 2.03 | EB, OVCA-R, A549 |
| 311099 | T56361 | Hs.182167 | hemoglobin: gamma A | 2.03 | HT29, RT474, EB |
| 316079 | AA922213 | Hs.121735 | ESTs | 2.03 | LnCap, OVCA-R, EB |
| 309533 | AW151131 | | EST singleton (not in UniGene) with exon | 2.03 | MB-MDA-231, BT474, LnCap |
| 338579 | | | CH22.EM:AC005500.GENSCAN.431-3 | 2.03 | NCI-H69, NCI-H345, RPWE-2 |
| 326549 | | | CH.19_hs gi\|5867307 | 2.03 | NCI-H69, Caco2, NCI-H345 |
| 320012 | AI628384 | Hs.193745 | ESTs | 2.03 | BT474, MB-MDA-453, MCF7 |
| 334111 | | | CH22_FGENES.330_10 | 2.03 | NCI-H69, MB-MDA-231, BT474 |
| 327123 | | | CH.21_hs gi\|6531971 | 2.03 | NCI-H345, NCI-H69, RPWE-2 |
| 324568 | AW502311 | | EST cluster (not in UniGene) | 2.03 | NCI-H345, NCI-H520, NCI-H460 |
| 306012 | AA896989 | | EST singleton (not in UniGene) with exon | 2.03 | NCI-H69, PRSC_log, PRSC_con |
| 303106 | AA012877 | | EST | 2.03 | RPWE-2, OVCA-R, EB |
| 302194 | U52219 | Hs.158329 | G protein-coupled receptor 50 | 2.03 | NCI-H520, NCI-H23, PC3 |
| 326646 | | | CH.20_hs gi\|5867562 | 2.03 | NCI-H460, OVCA-R, HT29 |
| 304060 | T61464 | | EST singleton (not in UniGene) with exon | 2.03 | NCI-H345, PRSC_con, PRSC_log |
| 304667 | AA535602 | | EST singleton (not in UniGene) with exon | 2.03 | A549, DU145, EB |
| 330514 | M83652 | Hs.53155 | properdin P factor, complement | 2.02 | NCI-H23, NCI-H460, NCI-358 |
| 310324 | AI473273 | Hs.159674 | ESTs; Weakly similar to GLUTAMATE [H.sap | 2.02 | NCI-H345, MB-MDA-231, BT474 |
| 330327 | | | CH.08_p2 gi\|5919194 | 2.02 | NCI-H345, NCI-H69, PRSC_log |
| 308447 | AI659985 | | EST singleton (not in UniGene) with exon | 2.02 | NCI-H345, RPWE-2, PRSC_log |
| 307778 | AI344972 | Hs.231496 | EST | 2.02 | NCI-H69, CALU6, OVCA-R |
| 319459 | T87351 | Hs.194121 | ESTs | 2.02 | NCI-H460, NCI-358, NCI-H520 |
| 300935 | AA513644 | Hs.222815 | ESTs; Weakly similar to Wiskott-Aldrich | 2.02 | DU145, EB, OVCA-R |
| 314318 | AL037405 | Hs.176141 | ESTs | 2.02 | PRSC_con, LnCap, PRSC_log |
| 334779 | | | CH22_FGENES.432_1 | 2.02 | EB, HT29, DU145 |
| 336994 | | | CH22_FGENES.410-2 | 2.02 | NCI-H345, PRSC_con, NCI-H69 |
| 334076 | | | CH22_FGENES.327_31 | 2.02 | OVCA-R, CALU6, EB |
| 318116 | AW452865 | Hs.132339 | ESTs | 2.02 | MB-MDA-231, NCI-H69, NCI-H345 |
| 326783 | | | CH.20_hs gi\|6525298 | 2.02 | NCI-H69, PRSC_con, RPWE-2 |
| 336142 | | | CH22_FGENEB.705_4 | 2.02 | NCI-H69, PRSC_log, PRSC_con |
| 320913 | AA663733 | | EST cluster (not in UniGene) | 2.02 | DU145, EB, CALU6 |
| 301644 | AW239364 | | EST | 2.02 | PRSC_con, RPWE-2, PRSC_log |
| 300944 | AW081072 | Hs.164624 | ESTs; Weakly similar to Slit-3 protein [ | 2.01 | RPWE-2, NCI-H69, NCI-H23 |
| 310080 | AW137088 | Hs.144857 | ESTs | 2.01 | PRSC_con, NCI-H345, PRSC_log |
| 311246 | AI863918 | Hs.195078 | ESTs | 2.01 | NCI-H345, NCI-H69, RPWE-2 |
| 319207 | R87679 | | EST cluster (not in UniGene) | 2.01 | HT29, A549, NCI-H460 |
| 334760 | | | CH22_FGENES.428_9 | 2.01 | NCI-358, NCI-H69, PRSC_log |
| 338368 | | | CH22_EM:AC005500.GENSCAN.325-2 | 2.01 | NCI-H23, NCI-H520, NCI-H460 |
| 317300 | AI417007 | Hs.166338 | ESTs | 2.01 | NCI-H460, DU145, NCI-H23 |
| 323699 | AW178750 | | EST cluster (not in UniGene) | 2.01 | MCF7, MB-MDA-453, OVCA-R |
| 301366 | AA907713 | Hs.221667 | ESTs | 2.01 | PRSC_con, NCI-H345, RPWE-2 |
| 333306 | | | CH22_FGENES.137_3 | 2.01 | NCI-H69, NCI-H345, PRSC_con |
| 328031 | | | CH.06_hs gi\|5902482 | 2.01 | MB-MDA-231, NCI-H345, PRSC_con |
| 301806 | AA326007 | Hs.12056 | asialoglycoprotein receptor 1 | 2.01 | MB-MDA-453, DU145, EB |
| 300993 | AA584930 | Hs.191777 | ESTs; Weakly similar to XAP-5-like prote | 2.01 | HT29, NCI-H23, NCI-358 |
| 320042 | T84520 | | EST cluster (not in UniGene) | 2.01 | PRSC_con, NCI-H345, NCI-H69 |
| 331082 | R17059 | Hs.22100 | ESTs | 2.01 | EB, DU145, MB-MDA-435s |
| 308851 | AI829820 | | EST singleton (not in UniGene) with exon | 2.01 | DU145, EB, PC3 |
| 301163 | AA732066 | | EST | 2.01 | OVCA-R, PC3, MB-MDA-435s |
| 304734 | AA576428 | | EST singleton (not in UniGene) with exon | 2.01 | LnCap, MB-MDA-453, DU145 |
| 334855 | | | CH22_FGENES.442_6 | 2.01 | NCI-H345, RPWE-2, PRSC_log |
| 337121 | | | CH22_FGENES.519-1 | 2.01 | NCI-H69, NCI-H345, PRSC_con |
| 331838 | AA412498 | Hs.104778 | ESTs | 2.01 | BT474, BT474, MCF7 |
| 339181 | | | CH22_DA59H18.GENSCAN.72-6 | 2.01 | NCI-H345, PRSC_con, NCI-H69 |
| 327564 | | | CH.03_hs gi\|5867811 | 2.01 | BT474, HT29, DU145 |
| 304108 | R63932 | Hs.28467 | EST | 2 | BT474, OVCA-R, MCF7 |
| 315036 | AA534953 | Hs.163297 | ESTs | 2 | MB-MDA-435s, MB-MDA-453, LnCap |
| 312777 | W92809 | Hs.138557 | ESTs | 2 | PRSC_con, NCI-H345, MB-MDA-231 |
| 305888 | AA868536 | Hs.126145 | EST | 2 | HT29, HT29, BT474 |
| 323185 | R52177 | | EST cluster (not in UniGene) | 2 | EB, A549, BT474 |
| 308681 | AI761307 | | EST singleton (not in UniGene) with exon | 2 | RPWE-2, PRSC_con, NCI-H345 |
| 325755 | | | CH.14_hs gi\|6682474 | 2 | NCI-H345, PRSC_con, PRSC_log |
| 324376 | AW499705 | | EST cluster (not in UniGene) | 2 | DU145, BT474, PC3 |
| 331890 | AA432166 | Hs.3577 | succinate dehydrogenase complex; subunit | 2 | CALU6, MB-MDA-453, A549 |

TABLE 4
Pkey: Unique Eos probeset identifier number
ExAccn: Exemplar Accession number, Genbank accession number
UnigeneID: Unigene number
Unigene Title: Unigene gene title

| | | | | Ratio | |
|---|---|---|---|---|---|
| Pkey | Exr_Accn | UniG_ID | Complete_Title | Met/BS | Top 3 expressing cell lines |
| 313166 | AI801098 | Hs.151500 | ESTs | 12.23 | Caco2, EB, OVCA-R |
| 334593 | | | CH22_FGENES.408_3 | 8.06 | NCI-H169, OVCA-R, OVCA-R |
| 331084 | R20655 | Hs.81281 | Human clone 23732 mRNA; partial cds | 7.89 | LnCap, OVCA-R, EB |
| 324598 | AA502659 | Hs.163986 | ESTs | 7.77 | OVCA-R, EB, CALU6 |
| 314071 | AA192455 | Hs.188690 | ESTs | 7.76 | CALU6, EB, DU145 |
| 315178 | AW362945 | Hs.162459 | ESTs | 6.81 | OVCA-R, EB, CALU6 |
| 325519 | | | CH.12_hs gi\|6017036 | 6.34 | NCI-H69, NCI-H345, PRSC_con |
| 331433 | H68097 | Hs.161023 | EST | 6.16 | OVCA-R, A549, EB |
| 315021 | AA533447 | | EST cluster (not in UniGene) | 6.15 | PC3, EB, CALU6 |
| 337695 | | | CH22_EM:AC000097.GENSCAN.84-1 | 5.84 | NCI-H69, NCI-H345, DU145 |
| 324048 | AA378739 | | EST cluster (not in UniGene) | 5.77 | OVCA-R, DU145, EB |
| 300781 | AA731209 | | EST cluster (not in UniGene) with exon h | 5.72 | MB-MDA-453, MCF7, MB-MDA-435s |
| 320701 | AI093177 | Hs.134923 | ESTs | 5.68 | A549, NCI-H345, NCI-H69 |
| 332471 | AA416967 | Hs.120980 | nuclear receptor co-repressor 2 | 5.68 | LnCap, A549, OVCA-R |
| 331858 | AA421163 | Hs.163848 | ESTs | 5.66 | OVCA-R, DU145, Caco2 |
| 330987 | H40988 | Hs.131965 | ESTs | 5.35 | NCI-H345, OVCA-R, LnCap |
| 322309 | AF086372 | | EST cluster (not in UniGene) | 5.31 | OVCA-R, DU145, PC3 |
| 324733 | AA582082 | Hs.199410 | ESTs | 5.17 | PRSC_con, PRSC_log, NCI-H345 |
| 313577 | AA565051 | Hs.155029 | ESTs | 5.16 | OVCA-R, PC3, EB |
| 310966 | AW271974 | Hs.210295 | ESTs | 5.15 | NCI-H69, PRSC_log, PRSC_con |
| 311332 | AW292247 | Hs.255052 | ESTs | 5.05 | Caco2, OVCA-R, EB |
| 314522 | AI732331 | Hs.187750 | ESTs; Moderately similar to !!!! ALU CLA | 5.04 | EB, DU145, HT29 |
| 330886 | AA135606 | Hs.189384 | ESTs; Weakly similar to !!!! ALU SUBFAMI | 4.93 | OVCA-R, DU145, Caco2 |
| 313597 | AW162263 | Hs.249990 | ESTs | 4.84 | NCI-H460, NCI-H345, NCI-H23 |
| 314439 | AI539443 | Hs.137447 | ESTs | 4.84 | DU145, Caco2, MB-MDA-231 |
| 320807 | AA086110 | Hs.188536 | H sapiens clone 24838 mRNA seq | 4.83 | PC3, OVCA-R, DU145 |
| 311804 | AA135159 | Hs.203349 | ESTs | 4.82 | OVCA-R, PC3, Caco2 |
| 321354 | AA078493 | | EST cluster (not in UniGene) | 4.81 | DU145, EB, OVCA-R |
| 325169 | H01560 | Hs.163818 | ESTs; Weakly similar to !!!! ALU SUBFAMI | 4.8 | NCI-H345, DU145, LnCap |
| 312828 | AI865455 | Hs.211818 | ESTs; Moderately similar to !!!! ALU SUB | 4.78 | DU145, DU145, DU145 |
| 321226 | AA311443 | Hs.251416 | H sapiens mRNA; cDNA DKFZp586E2317 (from | 4.75 | DU145, OVCA-R, MB-MDA-453 |
| 327772 | | | CH.05_hs gi\|5867964 | 4.74 | HT29, MB-MDA-231, NCI-H345 |
| 315642 | AA742222 | Hs.120634 | ESTs | 4.7 | DU145, EB, MB-MDA-453 |
| 311905 | AA555215 | Hs.151913 | ESTs | 4.7 | DU145, Caco2, PRSC_con |
| 312754 | R99834 | Hs.250383 | ESTs | 4.59 | OVCA-R, PC3, EB |
| 336637 | | | CH22_FGENES.13-7 | 4.58 | NCI-H69, PRSC_log, NCI-H345 |
| 331644 | T99544 | Hs.173734 | ESTs; Weakly similar to !!!! ALU CLASS B | 4.55 | OVCA-R, NCI-H345, Caco2 |
| 336984 | | | CH22_FGENES.401-2 | 4.55 | Caco2, Caco2, EB |
| 316261 | AW134485 | Hs.144967 | ESTs | 4.53 | NCI-H460, NCI-H345, Caco2 |
| 300417 | AW139492 | Hs.245887 | ESTs | 4.52 | DU145, CALU6, EB |
| 300610 | N72596 | Hs.99120 | DEAD/H (Asp-Glu-Ala-Asp/His) box polypep | 4.52 | OVCA-R, PC3, EB |
| 324718 | AI557019 | Hs.116467 | ESTs | 4.5 | LnCap, PC3, PRSC_con |
| 332170 | F04112 | Hs.177178 | ESTs | 4.47 | Caco2, DU145, DU145 |
| 324042 | AA377589 | | EST cluster (not in UniGene) | 4.45 | NCI-H345, PRSC_con, PRSC_log |
| 331148 | R73816 | Hs.17385 | ESTs | 4.44 | CALU6, OVCA-R, EB |
| 328981 | | | CH.09_hs gi\|5868527 | 4.43 | HT29, BT474, NCI-H69 |
| 321920 | N63915 | | EST cluster (not in UniGene) | 4.34 | Caco2, A549, A549 |
| 320832 | AA214584 | | EST cluster (not in UniGene) | 4.34 | NCI-H23, CALU6, OVCA-R |
| 321971 | AI680459 | Hs.201441 | ESTs | 4.33 | DU145, HT29, CALU6 |
| 308572 | AI707882 | | EST singleton (not in UniGene) with exon | 4.33 | MCF7, NCI-H345, OVCA-R |
| 302459 | AF169255 | | EST cluster (not in UniGene) with exon h | 4.28 | MB-MDA-231, OVCA-R, LnCap |
| 321847 | T08401 | | EST cluster (not in UniGene) | 4.25 | MB-MDA-453, MB-MDA-435s, MBS-MDA-231 |
| 337884 | | | CH22_EM:AC005500.GENSCAN.54-2 | 4.23 | HT29, NCI-H23, MB-MDA-435s |
| 307494 | AI269188 | Hs.175656 | EST | 4.23 | NCI-H23, NCI-H520, NCI-358 |
| 314915 | AA573072 | Hs.187748 | ESTs; Weakly similar to !!!! ALU SUSFAMI | 4.21 | PC3, OVCA-R, Caco2 |
| 336638 | | | CH22_FGENES.14-2 | 4.21 | NCI-H69, NCI-H345, PRSC_log |
| 319379 | T91443 | Hs.193963 | ESTs | 4.2 | PC3, OVCA-R, LnCap |
| 312332 | R33041 | Hs.106200 | ESTs | 4.19 | NCI-H69, OVCA-R, NCI-H460 |
| 331445 | H89093 | Hs.41215 | ESTs | 4.19 | EB, HT29, DU145 |
| 315841 | AW138397 | Hs.247572 | ESTs | 4.19 | Caco2, MB-MDA-453, LnCap |
| 315712 | AI950133 | Hs.120882 | ESTs; Moderately similar to !!!! ALU SUB | 4.18 | LnCap, NCI-H345, OVCA-R |
| 319559 | AA773876 | Hs.251597 | ESTs | 4.15 | NCI-H345, Caco2, DU145 |
| 300791 | AL138455 | Hs.256135 | ESTs; Moderately similar to !!!! ALU SUB | 4.13 | NCI-358, RPWE-2, NCI-H460 |
| 312129 | AW300867 | | EST cluster (not in UniGene) | 4.12 | OVCA-R, MCF7, A549 |
| 321166 | AA411263 | Hs.128783 | ESTs | 4.11 | QVCA-R, Caco2, PRSC_con |
| 313220 | AI971981 | Hs.118241 | ESTs | 4.1 | OVCA-R, DU145, Caco2 |
| 314022 | AW452420 | Hs.248678 | ESTs | 4.1 | OVCA-R, EB, PC3 |
| 321359 | AW474412 | | EST cluster (not in UniGene) | 4.1 | DU145, OVCA-R, PC3 |
| 328841 | | | CH.07_hs gi\|6381920 | 4.09 | NCI-H69, PRSC_log, NCI-H345 |
| 337898 | | | CH22_EM:AC005500.GENSCAN.56-5 | 4.09 | NCI-H345, NCI-H69, OVCA-R |
| 333245 | | | CH22_FGENES.115_2 | 4.09 | PRSC_log, PRSC_con, NCI-H345 |
| 311958 | AI247472 | Hs.132965 | ESTs | 4.06 | EB, DU145, CALU6 |
| 314775 | AI149880 | Hs.188809 | ESTs | 4.06 | OVCA-R, PC3, EB |
| 317901 | AW150944 | Hs.250541 | ESTs | 4.06 | BT474, MB-MDA-453, MB-MDA-435s |
| 309985 | AW452919 | | EST singleton (not in UniGene) with exon | 4.05 | MB-MDA-453, NCI-H23, NCI-H520 |
| 311004 | AA632846 | | EST cluster (not in UniGene) | 4.05 | MB-MDA-453, OVCA-R, EB |
| 323497 | AI523613 | Hs.221544 | ESTs | 4.04 | LnCap, OVCA-R, EB |
| 332347 | W60326 | Hs.221716 | ESTs | 4.04 | EB, CALU6, PC3 |
| 331388 | AA456852 | Hs.43543 | suppressor of whhe apricot homolog 2 | 4.01 | A549, EB, Caco2 |
| 313197 | AI738851 | Hs.222487 | ESTs | 3.96 | OVCA-R, EB, PC3 |
| 315710 | AA931550 | Hs.192785 | ESTs | 3.95 | EB, MB-MDA-231, OVCA-R |
| 316897 | AA838114 | | EST cluster (not in UniGene) | 3.94 | OVCA-R, A549, MB-MDA-453 |
| 322564 | W86440 | Hs.118344 | ESTs | 3.94 | NCI-H460, Caco2, EB |
| 304605 | AA513225 | | EST singleton (not in UniGene) with exon | 3.9 | NCI-H345, RPWE-2, BT474 |
| 325726 | | | CH.14_hs gi\|6552447 | 3.9 | OVCA-R, LnCap, LnCap |
| 320190 | R32047 | Hs.141012 | ESTs; Weakly similar to !!!! ALU SUBFAMI | 3.89 | DU145, NCI-H23, PRSC_log |
| 331566 | N63062 | Hs.48703 | EST | 3.87 | NCI-H23, NCI-H460, NCI-358 |
| 319403 | T98413 | | EST cluster (not in UniGene) | 3.86 | NCI-H345, PRSC_log, LnCap |
| 324643 | AI436356 | Hs.130729 | ESTs | 3.84 | OVCA-R, DU145, NCI-H345 |
| 315298 | AI969314 | Hs.211377 | ESTs | 3.82 | NCI-H345, PRSC_con, PRSC_log |
| 321632 | AA419617 | | EST cluster (not in UniGene) | 3.81 | EB, OVCA-R, A549 |
| 313219 | N74924 | Hs.182099 | ESTs | 3.8 | EB, Caco2, OVCA-R |
| 330833 | AA046804 | | ESTs; Weakly similar to !!!! ALU SUBFAMI | 3.8 | LnCap, DU145, PC3 |
| 327289 | | | CH.01_hs gi\|5867481 | 3.79 | EB, HT29, DU145 |
| 314429 | AW300749 | | EST cluster (not in UniGene) | 3.79 | OVCA-R, PC3, PRSC_con |
| 314475 | AI911160 | Hs.127505 | ESTs | 3.79 | DU145, CALU6, NCI-H69 |
| 317130 | AW293995 | Hs.192277 | ESTs | 3.78 | EB, PC3, Caco2 |
| 336635 | | | CH22_FGENES.13-5 | 3.77 | NCI-H69, NCI-H345, PRSC_log |
| 333323 | | | CH22_FGENES.138_16 | 3.76 | NCI-H460, NCI-H23, PRSC_con |
| 332135 | AA620331 | Hs.245351 | EST | 3.75 | NCI-H345, A549, Caco2 |
| 316979 | AA861087 | | EST cluster (not in UniGene) | 3.75 | NCI-H345, NCI-H69, RPWE-2 |
| 316435 | AI671871 | Hs.192618 | ESTs; Weakly similar to !!!! ALU CLASS C | 3.74 | MB-MDA-435s, MCF7, MB-MDA-453 |
| 316422 | AW135357 | Hs.192374 | ESTs | 3.73 | OVCA-R, A549, EB |
| 336616 | | | CH22_FGENES.613_5 | 3.72 | NCI-H69, NCI-H345, RPWE-2 |
| 320258 | W93241 | | EST cluster (not in UniGene) | 3.71 | MB-MDA-231, NCI-H69, EB |
| 300463 | N52510 | Hs.186470 | ESTs | 3.69 | OVCA-R, A549, DU145 |
| 306881 | AI088695 | | EST singleton (not in UniGene) with exon | 3.68 | CALU6, HT29, EB |
| 337304 | | | CH22_FGENES.681-6 | 3.67 | MCF7, MB-MDA-453, LnCap |
| 323693 | AW297758 | Hs.249721 | ESTs | 3.67 | OVCA-R, MB-MDA-453, DU145 |
| 331073 | R07998 | Hs.18628 | ESTs; Weakly similar to !!!! ALU SUBFAMI | 3.67 | RPWE-2, NCI-H345, OVCA-R |
| 318162 | AW296277 | Hs.132171 | ESTs | 3.67 | MB-MDA-231, DU145, CALU6 |
| 318042 | AW294522 | Hs.149991 | ESTs | 3.66 | ES, HT29, CALU6 |
| 308069 | AI470895 | | EST singleton (not in UniGene) with exon | 3.64 | Caco2, Caco2, NCI-H23 |
| 327614 | | | CH.04_hs gi\|6525283 | 3.62 | NCI-H460, NCI-H345, NCI-H69 |
| 337514 | | | CH22_FGENES.809.7 | 3.62 | NCI-358, NCI-H23, NCI-H460 |
| 332093 | AA608794 | Hs.112592 | ESTs | 3.6 | EB, OVCA-R, DU145 |
| 327793 | | | CH.05_hs gi\|5867979 | 3.59 | LnCap, OVCA-R, EB |
| 331053 | N70242 | Hs.183146 | ESTs | 3.59 | OVCA-R, EB, Caco2 |
| 303769 | AA134888 | Hs.173415 | ESTs | 3.58 | HT29, CALU6, CALU6 |
| 319872 | R97130 | Hs.189699 | ESTs | 3.58 | PRSC_con, LnCap, RPWE-2 |
| 317902 | AI828602 | Hs.211265 | ESTs | 3.57 | CALU6, NCI-H345, OVCA-R |
| 324090 | AI656531 | Hs.116070 | ESTs | 3.57 | PRSC_con, NCI-H345, PRSC_log |
| 300120 | AW204314 | Hs.170784 | ESTs | 3.57 | NCI-H69, NCI-H345, PRSC_con |
| 307752 | AI339447 | | EST singleton (not in UniGene) with exon | 3.56 | NCI-358, HT29, MB-MDA-231 |
| 322438 | W44531 | Hs.167851 | ESTs | 3.55 | NCI-H345, NCI-H69, Caco2 |
| 311275 | AI659166 | Hs.207144 | ESTs | 3.55 | MB-MDA-231, PRSC_con, LnCap |
| 338830 | | | CH22_DJ246D7.GENSCAN.6-7 | 3.54 | LnCap, PC3, OVCA-R |
| 315647 | AA648983 | Hs.212911 | ESTs | 3.53 | OVCA-R, MB-MDA-453, CALU6 |
| 331469 | N22273 | Hs.39140 | ESTs | 3.52 | EB, A549, CALU6 |
| 313445 | AI123657 | Hs.127264 | ESTs | 3.51 | EB, OVCA-R, A549 |
| 330139 | | | CH.21_432 gi\|4210430 | 3.5 | EB, CALU6, DU145 |
| 304450 | AA404521 | Hs.10326 | coatomer protein complex: subunit epsilo | 3.49 | NCI-H345, NCI-H69, NCI-H460 |
| 325763 | | | CH.14_hs gi\|6682475 | 3.49 | PC3, BT474, OVCA-R |
| 312803 | AA677934 | Hs.117864 | ESTs | 3.47 | OVCA-R, Caco2, MB-MDA-453 |
| 303654 | AA436942 | Hs.168308 | ESTs | 3.46 | DU145, NCI-H460, NCI-H69 |
| 317924 | AI222324 | Hs.166306 | ESTs; Weakly similar to zinc finger prot | 3.46 | PRSC_con, PRSC_log, NCI-H69 |
| 312354 | AA036955 | Hs.167040 | ESTs | 3.44 | Caco2, MB-MDA-435s, NCI-H460 |
| 337517 | | | CH22_FGENES.814-6 | 3.43 | NCI-H69, HT29, PC3 |
| 324865 | AA702138 | Hs.114103 | ESTs | 3.42 | NCI-H23, NCI-H460, NCI-H520 |
| 323755 | AW300094 | | EST cluster (not in UniGene) | 3.42 | PRSC_con, RPWE-2, NCI-H345 |
| 314452 | AL042699 | Hs.209222 | ESTs | 3.42 | NCI-H345, PRSC_con, PRSC_log |
| 337911 | | | CH22_EM:AC0055500.GENSCAN.59-6 | 3.42 | OVCA-R, PC3, HT29 |
| 318086 | AI025499 | Hs.132238 | ESTs | 3.41 | CALU6, LnCap, OVCA-R |
| 311859 | AA704705 | Hs.181044 | ESTs; Weakly similar to Chain A: Human O | 3.41 | LnCap, MB-MDA-435s, A549 |
| | | | Complexed With L-Canaline [H. sapiens] | | |
| 314409 | H15560 | Hs.131833 | ESTs | 3.41 | NCI-H69, LnCap, LnCap |
| 323333 | AA228883 | | EST cluster (not in UniGene) | 3.41 | Caco2, OVCA-R, NCI-H69 |
| 325690 | | | CH.14_hs gi\|5867021 | 3.4 | HT29, CALU6, DU145 |
| 314539 | AA398216 | Hs.190092 | ESTs | 3.4 | MB-MDA-231, BT474, EB |
| 310567 | AI691065 | Hs.155780 | ESTs | 3.4 | PRSC_con, NCI-H345, NCI-H69 |
| 330527 | S77356 | | transcript ch21 = oligomycin sensitivity c | 3.39 | NCI-H23, Caco2, A549 |
| | | | 8 stomach cancer cell lines, mRNA, 262 n | | |
| 314660 | AA436007 | Hs.188780 | ESTs | 3.39 | OVCA-R, BT474, Caco2 |
| 321321 | AB033072 | | EST cluster (not in UniGene) | 3.39 | NCI-358, EB, Caco2 |
| 323356 | AA234009 | Hs.188715 | ESTs | 3.38 | DU145, CALU6, CALU6 |
| 328592 | | | CH.07_ha gi\|5868227 | 3.38 | MCF7, NCI-358, MB-MDA-231 |
| 311116 | AI631195 | Hs.232193 | ESTs | 3.36 | NCI-H520, NCI-H23, PRSC_log |
| 323853 | AA393460 | | EST cluster (not in UniGene) | 3.36 | DU145, EB, Caco2 |
| 327740 | | | CH.05_hs gi\|5867943 | 3.35 | EB, LnCap, OVCA-R |
| 326857 | | | CH.20_hs gi\|6552460 | 3.33 | NCI-H69, MCF7, NCI-H345 |
| 317787 | AW339612 | Hs.249364 | ESTs | 3.31 | NCI-H345, PRSC_con, PRSC_log |
| 325760 | | | CH.14_hs gi\|6552449 | 3.3 | EB, CALU6, HT29 |
| 337513 | | | CH22_FGENES.809-4 | 3.29 | LnCap, NCI-H23, NCI-H460 |
| 336606 | | | CH22_FGENES.429_3 | 3.29 | NCI-H69, A549, NCI-H23 |
| 322895 | AW470295 | Hs.192152 | ESTs | 3.29 | DU145, Caco2, EB |
| 314312 | AA814971 | Hs.257634 | ESTs | 3.29 | RPWE-2, NCI-H69, NCI-H345 |
| 328224 | | | CH.06_hs gi\|5868101 | 3.28 | DU145, NCI-H345, LnCap |
| 336128 | | | CH22_FGENES.701_16 | 3.27 | BT474, NCI-H520, MB-MDA-231 |
| 332442 | AA281323 | Hs.4947 | ESTs | 3.27 | Caco2, PC3, NCI-H345 |
| 302514 | M14269 | | EST cluster (not in UniGene) with exon h | 3.27 | DU145, CALU6, NCI-H520 |
| 313749 | AW450376 | Hs.130803 | ESTs | 3.26 | OVCA-R, NCI-H69, DU145 |
| 302891 | AI681578 | Hs.114164 | ESTs | 3.26 | LnCap, NCI-H345, PRSC_log |
| 334690 | | | CH22_FGENES.420_3 | 3.25 | NCI-H69, RPWE-2, PRSC_con |
| 308676 | AI761036 | | EST singleton (not in UniGene) with exon | 3.25 | DU145, MB-MDA-231, HT29 |
| 304254 | AA046273 | Hs.111334 | ferritin; light polypeptide | 3.24 | OVCA-R, DU145, A549 |
| 311994 | AA648314 | Hs.13849 | ESTs | 3.24 | NCI-H460, NCI-H23, MB-MDA-453 |
| 321020 | AB023170 | Hs.227850 | KIAA0953 protein | 3.24 | EB, MCF7, MB-MDA-435s |
| 316724 | AA810788 | Hs.123337 | ESTs | 3.23 | DU145, OVCA-R, BT474 |
| 326942 | | | CH.21_hs gi\|6004446 | 3.22 | HT29, BT474, NCI-H23 |
| 324824 | AI826999 | Hs.224624 | ESTs | 3.21 | OVCA-R, MB-MDA-453, EB |
| 320789 | R78712 | | EST cluster (not in UniGene) | 3.21 | DU145, LnCap, EB |
| 315070 | AW131368 | Hs.185736 | ESTs | 3.21 | Caco2, NCI-358, NCI-H460 |
| 303794 | AW241987 | Hs.197025 | ESTs | 3.19 | OVCA-R, PC3, LnCap |
| 310237 | AI884313 | Hs.158906 | ESTs | 3.19 | NCI-358, NCI-H345, MCF7 |
| 313960 | AA130859 | | EST cluster (not in UniGene) | 3.18 | MB-MDA-231, HT29, BT474 |
| 336634 | | | CH22_FGENES.13-4 | 3.18 | NCI-H69, NCI-H345, BT474 |
| 301085 | AA779058 | Hs.190428 | ESTs; Weakly similar to NG26 [H. sapiens] | 3.17 | NCI-H345, NCI-H345, NCI-358 |
| 313774 | AW136836 | Hs.144583 | ESTs | 3.17 | Caco2, EB, OVCA-R |
| 307177 | AI188864 | | EST singleton (not in UniGene) with exon | 3.17 | EB, CALU6, CALU6 |
| 324025 | AI174861 | Hs.190623 | ESTs | 3.17 | OVCA-R, DU145, PC3 |
| 313099 | AI307359 | Hs.128064 | ESTs | 3.17 | MB-MDA-231, BT474, EB |
| 305536 | AA770682 | | EST singleton (not in UniGene) with exon | 3.17 | NCI-358, Caco2, HT29 |
| 331916 | AA446131 | Hs.124918 | ESTs | 3.17 | EB, OVCA-R, Caco2 |
| 314912 | AI431345 | Hs.161784 | ESTs | 3.17 | EB, BT474, MCF7 |
| 303388 | AL039604 | | EST cluster (not in UniGene) with exon h | 3.17 | HT29, NCI-358, Caco2 |
| 332273 | R05818 | Hs.173830 | ESTs | 3.16 | MCF7, DU145, EB |
| 314697 | AW088739 | Hs.243770 | ESTs | 3.16 | MB-MDA-453, DU145, MCF7 |
| 335344 | | | CH22_FGENES.536_3 | 3.15 | PRSC_log, NCI-H345, PRSC_con |
| 326162 | | | CH.17_hs gi\|5867168 | 3.15 | BT474, HT29, HT29 |
| 304467 | AA424703 | | EST singleton (not in UniGene) with exon | 3.15 | NCI-H23, RPWE-2, NCI-H460 |
| 339340 | | | CH22_BA354I12.GENSCAN.27-8 | 3.15 | LnCap, OVCA-R, MB-MDA-453 |
| 325393 | | | CH.12_hs gi\|5866921 | 3.13 | Caco2, NCI-H23, NCI-358 |
| 315367 | AA732484 | Hs.169399 | ESTs | 3.13 | OVCA-R, EB, MB-MDA-453 |
| 307085 | AI160868 | | EST singleton (not in UniGene) with exon | 3.12 | RPWE-2, PRSC_con, PRSC_log |
| 313001 | N29264 | Hs.249591 | ESTs; Moderately similar to !!!! ALU SUB | 3.12 | NCI-H345, OVCA-R, Caco2 |
| 307606 | AI290006 | | EST singleton (not in UniGene) with exon | 3.12 | MB-MDA-231, HT29, NCI-H23 |
| 325710 | | | CH.14_hs gi\|6682473 | 3.09 | NCI-H69, MB-MDA-453, BT474 |
| 313810 | AA400079 | Hs.257854 | ESTs | 3.09 | EB, DU145, CALU6 |
| 335482 | | | CH22_FGENES.570_11 | 3.09 | NCI-H460, NCI-358, NCI-H23 |
| 326310 | | | CH.17_hs gi\|5867277 | 3.08 | MCF7, MB-MDA-453, PC3 |
| 325742 | | | CH.14_hs gi\|6552448 | 3.08 | NCI-H23, NCI-H460, HT29 |
| 312467 | AI241809 | Hs.75458 | ribosomal protein L18 | 3.08 | NCI-358, NCI-H23, NCI-H460 |
| 327309 | | | CH.01_hs gi\|6456757 | 3.07 | NCI-H69, MB-MDA-435s, MB-MDA-435s |
| 310583 | AW205632 | Hs.211198 | ESTs | 3.07 | OVCA-R, A549, Caco2 |
| 322373 | W25673 | Hs.130829 | ESTs | 3.07 | NCI-H69, PRSC_con, NCI-H345 |
| 324497 | AW152624 | Hs.136340 | ESTs | 3.06 | NCI-H345, RPWE-2, PRSC_con |
| 315095 | AA831815 | Hs.243788 | ESTs | 3.06 | Caco2, DU145, EB |
| 302445 | N79647 | | EST cluster (not in UniGene) with exon h | 3.05 | OVCA-R, A549, NCI-H460 |
| 302842 | AW383226 | Hs.163834 | ESTs; Highly similar to Chp [R. norvegicu | 3.05 | A549, DU145, NCI-H23 |
| 317346 | AA952875 | Hs.221274 | ESTs | 3.04 | BT474, HT29, HT29 |
| 334650 | | | CH22_FGENES.417_17 | 3.04 | MCF7, BT474, OVCA-R |
| 306644 | AI002913 | | EST singleton (not in UniGene) with exon | 3.04 | CALU6, MCF7, BT474 |
| 322682 | AI110679 | | EST cluster (not in UniGene) | 3.03 | NCI-H345, RPWE-2, OVCA-R |
| 311065 | AW204582 | Hs.224906 | ESTs | 3.03 | PRSC_log, PRSC_con, NCI-H460 |
| 318623 | AA355439 | Hs.151547 | ESTs | 3.03 | DU145, MB-MDA-435s, HT29 |
| 304978 | AA617735 | | EST singleton (not in UniGene) with exon | 3.03 | CALU6, BT474, MB-MDA-435s |
| 305554 | AA774567 | Hs.121774 | EST | 3.03 | EB, NCI-H460, Caco2 |
| 302574 | U66199 | Hs.249165 | fibroblast growth factor 11 | 3.03 | HT29, DU145, PC3 |
| 336202 | | | CH22_FGENES.719_6 | 3.02 | NCI-H69, NCI-H23, NCI-H23 |
| 302893 | AL117539 | Hs.173515 | H sapiens mRNA; cDNA DKFZp586H021 (from | 3.02 | EB, DU145, CALU6 |
| 315166 | AI343966 | Hs.158528 | ESTs | 3.01 | Caco2, EB, NCI-H69 |
| 335606 | | | CH22_FGENES.582_3 | 3.01 | NCI-H23, NCI-H520, NCI-H345 |
| 330058 | | | CH.17_p2 gi\|6634847 | 3.01 | OVCA-R, HT29, LnCap |
| 303179 | AA071215 | | EST cluster (not in UniGene) with exon h | 3.01 | MCF7, RPWE-2, MB-MDA-453 |
| 307625 | AI299617 | | EST singleton (not in UniGene) with exon | 3 | MB-MDA-231, LnCap, BT474 |
| 323074 | AL119445 | Hs.203213 | ESTs | 3 | NCI-H23, NCI-H520, NCI-H460 |
| 336232 | | | CH22_FGENES.736_7 | 3 | HT29, BT474, MB-MDA-231 |
| 334915 | | | CH22_FGENES.457_4 | 3 | NCI-H345, PRSC_con, NCI-H69 |
| 329116 | | | CH.X_hs gi\|5868650 | 3 | NCI-H69, PRSC_con, RPWE-2 |
| 333495 | | | CH22_FGENES.168_5 | 3 | OVCA-R, NCI-H169, NCI-H345 |
| 303756 | AI738488 | Hs.115838 | ESTs | 2.99 | HT29, PRSC_con, DU145 |
| 332134 | AA610123 | Hs.139240 | DKFZP564F1422 protein | 2.99 | EB, A549, MCF7 |
| 322916 | AW367294 | Hs.154091 | ESTs | 2.99 | DU145, DU145, OVCA-R |
| 318050 | AI052093 | Hs.133132 | ESTs | 2.99 | NCI-H345, DU145, NCI-H520 |
| 301019 | AI147356 | Hs.98722 | ESTs | 2.99 | NCI-358, NCI-H69, MB-MDA-435s |
| 315213 | AA587773 | Hs.136494 | ESTs | 2.98 | MB-MDA-231, BT474, LnCap |
| 339251 | | | CH22_BA354I12.GENSCAN.7-5 | 2.98 | NCI-H69, PRSC_log, HT29 |
| 303835 | T05645 | | EST cluster (not in UniGene) with exon h | 2.97 | BT474, NCI-H345, LnCap |
| 300070 | AI174603 | Hs.256832 | ESTs | 2.97 | DU145, A549, OVCA-R |
| 320954 | AB028953 | Hs.204121 | KIAA1030 protein | 2.97 | LnCap, DU145, PC3 |
| 327624 | | | CH.04_hs gi\|5867871 | 2.97 | EB, DU145, LnCap |
| 329029 | | | CH.X_hs gi\|6525302 | 2.96 | NCI-H69, PRSC_log, LnCap |
| 317040 | AA868584 | Hs.126154 | ESTs | 2.96 | DU145, EB, LnCap |
| 328016 | | | CH.06_hs gi\|5902482 | 2.96 | NCI-H345, PRSC_con, DU145 |
| 312674 | AI762475 | Hs.151327 | ESTs; Moderately similar to !!!! ALU SUB | 2.96 | OVCA-R, NCI-H69, NCI-H69 |
| 332301 | R70253 | Hs.127826 | ESTs | 2.96 | OVCA-R, DU145, MB-MDA-231 |
| 300951 | AI732374 | Hs.105834 | ESTs; Weakly similar to 25 kDa trypsin i | 2.95 | NCI-358, NCI-H460, Caco2 |
| 318226 | AI078446 | Hs.134125 | ESTs | 2.95 | NCI-H460, NCI-H23, NCI-358 |
| 311349 | AW292933 | Hs.254110 | ESTs | 2.94 | EB, DU145, OVCA-R |
| 312757 | AI285970 | Hs.183817 | ESTs | 2.94 | DU145, LnCap, LnCap |
| 316507 | AI381515 | Hs.158381 | ESTs | 2.94 | PRSC_con, PRSC_log, RPWE-2 |
| 302278 | AF018080 | Hs.173730 | Mediterranean fever | 2.93 | EB, NCI-H69, DU145 |
| 311016 | AW173166 | Hs.243468 | ESTs | 2.93 | NCI-H345, LnCap, LnCap |
| 323864 | AA340724 | Hs.214028 | ESTs | 2.92 | EB, Caco2, HT29 |
| 336632 | | | CH22_FGENES.13-2 | 2.92 | NCI-H69, NCI-H345, MB-MDA-231 |
| 328886 | | | CH.07_hs gi\|6588003 | 2.92 | HT29, PC3, LnCap |
| 301859 | T61587 | | EST cluster (not in UniGene) with exon h | 2.92 | LnCap, EB, EB |
| 323775 | AA329856 | Hs.143022 | ESTs | 2.92 | PRSC_con, PRSC_log, RPWE-2 |
| 315426 | AI391486 | Hs.128171 | ESTs | 2.92 | CALU6, EB, A549 |
| 322264 | AF086242 | | EST cluster (not in UniGene) | 2.92 | Caco2, OVCA-R, DU145 |
| 315135 | AA627561 | Hs.192446 | ESTs | 2.91 | EB, HT29, DU145 |
| 327982 | | | CH.06_hs gi\|5868216 | 2.91 | LnCap, MS-MDA-453, NCI-H169 |
| 314530 | AI052358 | Hs.131741 | ESTs | 2.91 | NCI-H460, NCI-H520, RPWE-2 |
| 315003 | AA527650 | Hs.156037 | ESTs | 2.9 | PRSC_con, RPWE-2, MB-MDA-231 |
| 339032 | | | CH22_DA59H18.GENSCAN.25-1 | 2.9 | NCI-H69, PRSC_con, RPWE-2 |
| 308379 | AI623950 | Hs.2186 | eukaryotic translation elongation factor | 2.89 | BT474, MB-MDA-231, HT29 |
| 312133 | T87714 | Hs.221665 | ESTs | 2.88 | Caco2, MB-MDA-453, MCF7 |
| 307992 | AI434166 | | EST singleton (not in UniGene) with exon | 2.88 | NCI-H520, MCF7, NCI-H23 |
| 308010 | AI439190 | Hs.181165 | eukaryotic translation elongation factor | 2.88 | Caco2, NCI-H69, NCI-H345 |
| 320154 | AA336019 | Hs.119559 | ESTs | 2.88 | MB-MDA-453, DU145, EB |
| 331496 | N34929 | Hs.171984 | ESTs | 2.86 | MB-MDA-453, PC3, MCF7 |
| 320016 | H57622 | Hs.194574 | ESTs | 2.86 | PRSC_con, RPWE-2, PRSC_log |
| 317923 | AW450544 | Hs.220751 | ESTs | 2.86 | NCI-H345, PRSC_con, PRSC_log |
| 301822 | X17033 | Hs.1142 | integrin; alpha 2 (CD49B: alpha 2 subuni | 2.86 | PC3, BT474, CALU6 |
| 311759 | AA705075 | Hs.169536 | Rhesus blood group-associated glycoprote | 2.85 | DU145, HT29, MB-MDA-231 |
| 315083 | AI221325 | Hs.210655 | ESTs | 2.84 | PRSC_con, RPWE-2, NCI-H345 |
| 317759 | AI908455 | Hs.202460 | ESTs; Weakly similar to hypothetical L1 | 2.83 | HT29, MB-MDA-231, BT474 |
| 313980 | AI633205 | Hs.159914 | ESTs | 2.83 | Caco2, MB-MDA-453, A549 |
| 310941 | AI453402 | Hs.173705 | ESTs; Weakly similar to !!!! ALU CLASS C | 2.83 | NCI-H345, MCF7, Caco2 |
| 313593 | AI911488 | Hs.213724 | ESTs | 2.83 | LnCap, Caco2, NCI-H460 |
| 314973 | AW273128 | Hs.254669 | EST | 2.82 | BT474, LnCap, RPWE-2 |
| 310950 | AI582758 | Hs.170561 | ESTs | 2.82 | EB, MB-MDA-453, LnCap |
| 323626 | AL039822 | Hs.207604 | ESTs | 2.82 | PC3, HT29, CALU6 |
| 325410 | | | CH.12_hs gi\|5866921 | 2.81 | MB-MDA-453, PRSC_con, NCI-358 |
| 313911 | AI565458 | Hs.116385 | ESTs | 2.81 | PRSC_con, EB, RPWE-2 |
| 334244 | | | CH22_GENES.365_5 | 2.81 | OVCA-R, PC3, MB-MDA-453 |
| 309333 | AW025709 | | EST singleton (not in UniGene) with exon | 2.81 | NCI-H460, NCI-H23, NCI-358 |
| 328467 | | | CH.07_hs gi\|5868434 | 2.81 | EB, OVCA-R, HT29 |
| 318563 | AW250501 | | EST cluster (not in UniGene) | 2.81 | BT474, NCI-H23, MB-MDA-231 |
| 326412 | | | CH.19_hs gi\|5867362 | 2.81 | BT474, PRSC_log, RPWE-2 |
| 303407 | AA309616 | | EST cluster (not in UniGene) with exon h | 2.8 | CALU6, NCI-H345, DU145 |
| 328462 | | | CH.07_hs gi\|5868433 | 2.8 | BT474, CALU6, MCF7 |
| 335157 | | | CH22_FGENES.501_7 | 2.8 | NCI-H69, NCI-H345, PRSC_log |
| 313458 | AA007259 | Hs.255853 | ESTs | 2.79 | OVCA-R, DU145, LnCap |
| 310416 | AI695047 | Hs.202395 | ESTs | 2.79 | DU145, MB-MDA-435s, PC3 |
| 317709 | AI435973 | Hs.128056 | ESTs | 2.79 | NCI-H460, NCI-358, DU145 |
| 321415 | AI377596 | Hs.3337 | transmembrane 4 superfamily member 1 | 2.79 | A549, PC3, OVCA-R |
| 313693 | AW469180 | Hs.170651 | ESTs | 2.79 | OVCA-R, MCF7, EB |
| 309438 | AW102802 | Hs.225787 | ESTs; Moderately similar to hypothetical | 2.79 | PC3, OVCA-R, DU145 |
| 308961 | AI870248 | | EST singleton (not in UniGene) with exon | 2.78 | BT474, MB-MDA-231, EB |
| 329107 | | | CH.X_hs gi\|5868626 | 2.78 | DU145, MCF7, MB-MDA-435s |
| 313975 | AW025024 | Hs.65114 | keratin 18 | 2.78 | Caco2, EB, DU145 |
| 330901 | AA157818 | Hs.238380 | Human endogenous retroviral pretease mRN | 2.78 | PC3, NCI-H520, BT474 |
| 311749 | R06249 | Hs.13911 | ESTs | 2.78 | OVCA-R, MB-MDA-453, MCF7 |
| 329853 | | | CH.14_p2 gi\|6682295 | 2.78 | BT474, BT474, HT29 |
| 322340 | AF088076 | | EST cluster (not in UniGene) | 2.77 | NCI-H345, Caco2, LnCap |
| 326806 | | | CH.20_hs gi\|6469835 | 2.77 | NCI-H69, NCI-H345, MB-MDA-231 |
| 314661 | AA436432 | | EST cluster (not in UniGene) | 2.77 | NCI-H460, MB-MDA-435s, CALU6 |
| 322135 | AF075082 | | EST cluster (not in UniGene) | 2.77 | NCI-358, NCI-H460, Caco2 |
| 331849 | AA417078 | Hs.193767 | ESTs | 2.77 | DU145, EB, CALU6 |
| 301056 | AI797955 | Hs.208076 | ESTs; Weakly similar to D(4) DOPAMINE RE | 2.76 | NCI-H69, RPWE-2, PRSC_con |
| 327739 | | | CH.05_hs gi\|5867942 | 2.76 | EB, PC3, LnCap |
| 308016 | AI445116 | | EST singleton (not in UniGene) with exon | 2.76 | LnCap, HT29, MB-MDA-231 |
| 331549 | N56866 | Hs.237507 | EST | 2.76 | MB-MDA-453, MCF7, OVCA-R |
| 331851 | AA418599 | Hs.98303 | caveolin 3 | 2.75 | MB-MDA-231, NCI-H345, BT474 |
| 315023 | AA533505 | Hs.185844 | ESTs | 2.75 | PRSC_con, OVCA-R, EB |
| 335565 | | | CH22_FGENES.579_1 | 2.75 | OVCA-R, EB, A549 |
| 306137 | AA916176 | | EST singleton (not in UniGene) with exon | 2.74 | EB, LnCap, DU145 |
| 332240 | N54803 | | yv31d2.s1 Soares fetal liver spleen 1NFL | 2.74 | DU145, EB, CALU6 |
| | | | 3′ similar to contains L1.t3 L1 repetit | | |
| 313246 | N90762 | Hs.159454 | ESTs | 2.74 | NCI-H69, NCI-H345, PRSC_log |
| 303642 | AW299459 | | EST cluster (not in UniGene) with exon h | 2.74 | EB, A549, Caco2 |
| 325513 | | | CH.12_hs gi\|6017035 | 2.74 | MB-MDA-231, NCI-H345, BT7474 |
| 337236 | | | CH22_FGENES.639-2 | 2.74 | MCF7, MB-MDA-453, NCI-H69 |
| 311555 | AW407892 | Hs.244807 | ESTs | 2.74 | BT474, NCI-H345, NCI-H69 |
| 339266 | | | CH22_BA354I12.GENSCAN.10-4 | 2.73 | CALU6, DU145, OVCA-R |
| 300127 | AW028615 | Hs.235224 | ESTs; Weakly similar to KIAA0422 [H. sapi | 2.73 | NCI-H345, RPWE-2, PRSC_log |
| 311741 | R00099 | Hs.193642 | ESTs | 2.72 | LnCap, PC3, OVCA-R |
| 310915 | AW449673 | Hs.201893 | ESTs | 2.72 | DU145, EB, MB-MDA-435s |
| 324982 | T31689 | Hs.98518 | ESTs | 2.71 | PRSC_con, PRSC_log, RPWE-2 |
| 305030 | AA629988 | | EST singleton (not in UniGene) with exon | 2.71 | DU145, DU145, NCI-358 |
| 315396 | AW296107 | Hs.152686 | ESTs | 2.69 | OVCA-R, Oaco2, EB |
| 319098 | AI908374 | | EST cluster (not in UniGene) | 2.69 | RPWE-2, LnCap, PC3 |
| 309119 | AI927384 | Hs.228499 | EST; Moderately similar to PK-120 precur | 2.69 | LnCap, NCI-H23, NCI-358 |
| 312095 | AW444937 | Hs.233482 | ESTs | 2.68 | Caco2, OVCA-R, HT29 |
| 324316 | AI291330 | | EST cluster (not in UniGene) | 2.68 | NCI-H460, Caco2, PRSC_log |
| 331367 | AA425688 | Hs.41641 | ESTs; Weakly similar to CAGH4 [H. sapiens | 2.68 | MB-MDA-435s, NCI-H520, NCI-H460 |
| 339116 | | | CH22_DA59H18.GENSCAN.49-4 | 2.68 | DU145, EB, CALU6 |
| 324297 | AI565566 | Hs.168587 | ESTs | 2.68 | PRSC_con, OVCA-R, PRSC_log |
| 318728 | Z30201 | | EST cluster (not in UniGene) | 2.68 | LnCap, Caco2, PC3 |
| 304813 | AA584540 | | EST singleton (not in UniGene) with exon | 2.68 | BT474, OVCA-R, RPWE-2 |
| 312393 | N34376 | Hs.191659 | ESTs; Weakly similar to !!!! ALU CLASS E | 2.68 | NCI-H345, PRSC_con, EB |
| 330671 | AB002302 | Hs.92236 | KIAA0304 gene product | 2.67 | NCI-358, OVCA-R, Caco2 |
| 305406 | AA723860 | | EST singleton (not in UniGene) with exon | 2.66 | OVCA-R, EB, MCF7 |
| 330957 | H08778 | Hs.133521 | ESTs | 2.66 | EB, PC3, OVCA-R |
| 300350 | AI871129 | Hs.172597 | ESTs; Weakly similar to zinc finger prot | 2.66 | NCI-H23, NCI-H520, NCI-H460 |
| 322302 | W76021 | | EST cluster (not in UniGene) | 2.66 | DU145, OVCA-R, PC3 |
| 321891 | AW157424 | Hs.165954 | ESTs | 2.66 | EB, OVCA-R, Caco2 |
| 300124 | AI217394 | Hs.242447 | ESTs | 2.65 | PRSC_con, A549, HT29 |
| 302747 | AF062275 | | EST cluster (not in UniGene) with exon h | 2.65 | NCI-H23, BT474, MCF7 |
| 309741 | AI802780 | Hs.209002 | ESTs; Weakly similar to !!!! ALU SUBFAMI | 2.65 | PC3, EB, OVCA-R |
| 310802 | AI631546 | Hs.159732 | ESTs | 2.65 | PRSC_con, PRSC_log, NCI-H69 |
| 300694 | AA063406 | | EST cluster (not in UniGene) with exon h | 2.65 | BT474, EB, MCF7 |
| 311395 | R23313 | | EST cluster (not in UniGene) | 2.64 | EB, OVCA-R, DU145 |
| 336538 | | | CH22_FGENES.840_2 | 2.64 | DU145, NCI-H460, NCI-358 |
| 316473 | AA829961 | | EST cluster (not in UniGene) | 2.64 | LnCap, OVCA-R, EB |
| 328134 | | | CH.06_hs gi\|5868039 | 2.64 | LnCap, EB, CALU6 |
| 329330 | | | CH.X_hs gi\|5868806 | 2.64 | EB, CALU6, DU145 |
| 316664 | AI042101 | | EST cluster (not in UniGene) | 2.64 | NCI-H345, MB-MDA-231, PRSC_log |
| 328015 | | | CH.06_hs gi\|5902482 | 2.63 | BT474, HT29, MB-MDA-231 |
| 308991 | AI879831 | | EST singleton (not in UniGene) with exon | 2.63 | BT474, EB, NCI-H23 |
| 323899 | AL042966 | | EST cluster (not in UniGene) | 2.62 | DU145, A549, CALU6 |
| 321708 | AA476817 | | EST cluster (not in UniGene) | 2.62 | EB, A549, CALU6 |
| 301752 | T75247 | | EST cluster (not in UniGene) with exon h | 2.62 | HT29, BT474, NCI-H345 |
| 309351 | AW057547 | | EST singleton (not in UniGene) with exon | 2.62 | NCI-H23, PRSC_con, LnCap |
| 314412 | AI864270 | Hs.155654 | ESTs | 2.62 | CALU6, MB-MDA-231, BT474 |
| 309441 | AW103055 | Hs.244230 | EST | 2.62 | BT474, MB-MDA-231, MB-MDA-453 |
| 335993 | | | CH22_FGENES.656_6 | 2.61 | NCI-H460, NCI-358, NCI-H520 |
| 318196 | AI056776 | Hs.133397 | ESTs | 2.6 | EB, CALU6, HT29 |
| 322880 | AA310521 | Hs.50848 | ESTs; Weakly similar to KIAA0862 protein | 2.6 | DU145, A549, PC3 |
| 300558 | AI540051 | Hs.122638 | ESTs | 2.6 | OVCA-R, NCI-H69, MCF7 |
| 318594 | AA918320 | Hs.224581 | ESTs | 2.6 | PC3, MB-MDA-453, DU145 |
| 308554 | AI698132 | Hs.201923 | EST | 2.6 | LnCap, EB, NCI-H345 |
| 335108 | | | CH22_FGENES.494_14 | 2.6 | NCI-H69, NCI-H345, MB-MDA-231 |
| 312483 | AI417526 | Hs.184636 | ESTs | 2.59 | PC3, DU145, OVCA-R |
| 311981 | AW452773 | Hs.257612 | EST | 2.59 | NCI-H460, MB-MDA-453, NCI-H23 |
| 319359 | F13458 | | EST cluster (not in UniGene) | 2.59 | LnCap, NCI-H460, MB-MDA-231 |
| 300230 | AI377746 | Hs.158846 | ESTs | 2.59 | HT29, NCI-358, NCI-H345 |
| 316504 | AW135854 | Hs.132458 | ESTs | 2.59 | DU145, EB, CALU6 |
| 322337 | AA249804 | | EST cluster (not in UniGene) | 2.59 | NCI-H69, NCI-H345, NCl-H345 |
| 301775 | AW247670 | | EST cluster (not in UniGene) with exon h | 2.59 | NCI-H345, RPWE-2, PRSC_log |
| 301089 | AA666396 | Hs.220727 | ESTs | 2.58 | PRSC_log, PRSC_con, RPWE-2 |
| 331213 | T88698 | Hs.163862 | ESTs | 2.58 | DU145, EB, OVCA-R |
| 321121 | W23285 | | EST cluster (not in UniGene) | 2.58 | NCI-H69, MB-MDA-435s, PC3 |
| 316634 | AW241910 | Hs.122254 | ESTs | 2.58 | MCF7, HT29, BT474 |
| 322141 | AF075092 | | EST cluster (not in UniGene) | 2.58 | PC3, OVCA-R, HT29 |
| 312108 | T82331 | Hs.127453 | ESTs | 2.58 | A549, CALU6, Caco2 |
| 339071 | | | CH22_DA59H18.GENSCAN.34-1 | 2.58 | CALU6, DU145, EB |
| 311666 | AW389509 | Hs.223747 | ESTs | 2.57 | OVCA-R, MB-MDA-231, BT474 |
| 318662 | AI285898 | Hs.115367 | ESTs | 2.57 | OVCA-R, DU145, EB |
| 317010 | AA863395 | | EST cluster (not in UniGene) | 2.57 | NCI-H520, PRSC_con, NCI-358 |
| 324710 | AI742028 | Hs.120884 | ESTs; Weakly similar to RAS-RELATED PROT | 2.57 | LnCap, DU145, MB-MDA-453 |
| 327888 | | | CH.06_hs gi\|5868149 | 2.56 | NCI-H345, MB-MDA-435s, RPWE-2 |
| 336149 | | | CH22_FGENES.706.5 | 2.56 | NCI-H69, PC3, A549 |
| 312816 | H74319 | Hs.188620 | ESTs | 2.56 | EB, Caco2, NCI-H460 |
| 327999 | | | CH.06_hs gi\|5867994 | 2.56 | NCI-358, NCI-H520, NCI-H23 |
| 316761 | AI911173 | Hs.213722 | ESTs | 2.55 | NCI-H345, NCI-H460, MB-MDA-231 |
| 336958 | | | CH22_FGENES.367-1 | 2.55 | HT29, CALU6, CALU6 |
| 325043 | W27919 | Hs.32944 | inositol polyphosphate-4-phosphatase; ty | 2.55 | NCI-H460, NCI-H23, HT29 |
| 315417 | AW452360 | Hs.186770 | ESTs | 2.55 | NCI-H345, NCI-H69, PRSC_con |
| 331603 | N78656 | Hs.161535 | EST | 2.55 | NCI-H345, PRSC_con, PRSC_log |
| 309403 | AW082954 | | EST singleton (not in UniGene) with exon | 2.55 | BT474, MB-MDA-231, MCF7 |
| 337289 | | | CH22_FGENES.672-8 | 2.54 | BT474, HT29, MB-MDA-231 |
| 314242 | AI570943 | Hs.246280 | ESTs | 2.54 | Caco2, MB-MDA-435s, MB-MDA-453 |
| 328053 | | | CH.06_hs gi\|5902482 | 2.54 | MB-MDA-231, DU145, MB-MDA-453 |
| 307215 | AI193189 | | EST singleton (not in UniGene) with exon | 2.53 | HT29, CALU6, MB-MDA-231 |
| 327566 | | | CH.03_hs gi\|5867811 | 2.53 | NCI-H69, NCI-H520, NCI-H345 |
| 326338 | | | CH.17_hs gi\|6056311 | 2.53 | PC3, A549, DU145 |
| 318115 | AI384027 | Hs.159130 | ESTs; Moderately similar to !!!! ALU SUB | 2.53 | DU145, EB, PC3 |
| 307437 | AI245683 | | EST singleton (not in UniGene) with exon | 2.52 | NCI-H23, NCI-H520, NCI-358 |
| 322059 | AA412371 | Hs.121344 | ESTs | 2.52 | EB, DU145, OVCA-R |
| 322505 | AF147315 | | EST cluster (not in UniGene) | 2.52 | PRSC_con, RPWE-2, NCI-H69 |
| 314032 | AW081897 | Hs.193211 | ESTs | 2.52 | NCI-H345, LnCap, DU145 |
| 336125 | | | CH22_FGENES.701_12 | 2.51 | NCI-H69, LnCap, DU145 |
| 312765 | AI692908 | Hs.181873 | ESTs | 2.51 | NCI-H23, NCI-358, NCI-H520 |
| 335523 | | | CH22_FGENES.572_3 | 2.51 | HT29, BT474, OVCA-R |
| 327585 | | | CH.03_hs gi\|5867825 | 2.51 | HT29, NCI-H460, MB-MDA-453 |
| 323183 | AW393850 | | EST cluster (not in UniGene) | 2.51 | MB-MDA-231, LnCap, RPWE-2 |
| 314418 | AI478722 | Hs.232275 | ESTs; Moderately similar to !!!! ALU SUB | 2.51 | EB, DU145, DU145 |
| 313361 | AI359782 | Hs.137312 | ESTs | 2.5 | CALU6, HT29, DU145 |
| 305632 | AA805276 | | EST singleton (not in UniGene) with exon | 2.5 | MB-MDA-453, NCI-H460, NCI-H23 |
| 331689 | W90131 | Hs.184675 | ESTs | 2.5 | NCI-H69, EB, A549 |
| 323438 | AI540243 | Hs.113817 | ESTs | 2.5 | NCI-H345, PRSC_con, MB-MDA-231 |
| 315742 | AI821724 | Hs.143198 | H sapiens PAC clone DJ0872F07 from 7q31 | 2.5 | MCF7, MB-MDA-453, MB-MDA-435s |
| 305971 | AA886874 | | EST singleton (not in UniGene) with exon | 2.5 | NCI-358, NCI-H23, NCI-H520 |
| 336633 | | | CH22_FGENES.13-3 | 2.5 | NCI-H69, NCI-H345, PRSC_log |
| 304746 | AA577793 | | EST singleton (not in UniGene) with exon | 2.49 | NCI-H69, BT474, MB-MDA-231 |
| 327925 | | | CH.06_hs gi\|5868172 | 2.49 | NCI-358, NCI-358, NCI-H460 |
| 336055 | | | CH22_FGENES.683_4 | 2.49 | EB, HT29, MB-MDA-231 |
| 328888 | | | CH.07_hs gi\|6588003 | 2.48 | MB-MDA-435s, MB-MDA-453, PRSC_log |
| 311244 | AW016694 | Hs.197689 | ESTs | 2.48 | NCI-H345, MCF7, PC3 |
| 327155 | | | CH.01_hs gi\|5867549 | 2.48 | NCI-H69, MB-MDA-231, NCI-H345 |
| 334907 | | | CH22_FGENES.453_2 | 2.48 | DU145, NCI-H345, MB-MDA-231 |
| 314887 | AA910236 | Hs.139469 | ESTs | 2.48 | DU145, A549, A549 |
| 339435 | | | CH22_DJ579N16.GENSCAN.18-10 | 2.48 | NCI-H69, MCF7, BT474 |
| 334172 | | | CH22_FGENES.349_5 | 2.48 | NCI-H69, NCI-H345, PRSC_log |
| 320767 | AA299525 | | EST cluster (not in UniGene) | 2.48 | NCI-358, NCI-H23, NCI-H460 |
| 338772 | | | CH22_FGENES.156-1 | 2.47 | NCI-358, NCI-358, NCI-H23 |
| 326957 | | | CH.21_hs gi\|6469836 | 2.47 | BT474, RPWE-2, PRSC_con |
| 308505 | AI686615 | Hs.200778 | EST; Weakly similar to SALIVARY PROLINE- | 2.47 | MCF7, MB-MDA-453, MB-MDA-435s |
| 321325 | AB033100 | | EST cluster (not in UniGene) | 2.47 | EB, CALU6, A549 |
| 313149 | AW291092 | Hs.201058 | ESTs | 2.47 | NCI-H345, PRSC_con, RPWE-2 |
| 338325 | | | CH22_EM:AC005500.GENSCAN.307-7 | 2.46 | BT474, LnCap, EB |
| 307877 | AI368880 | | EST singleton (not in UniGene) with exon | 2.46 | NCI-H23, PRSC_log, NCI-H520 |
| 311525 | AI799444 | Hs.247095 | ESTs; Moderately similar to !!!! ALU SUB | 2.46 | PRSC_con, PRSC_log, NCI-H345 |
| 337023 | | | CH22_FGENES.433-12 | 2.46 | OVCA-R, CALU6, PRSC_con |
| 300916 | AI361798 | Hs.164675 | ESTs | 2.45 | LnCap, DU145, CALU6 |
| 302919 | AL137382 | | EST cluster (not in UniGene) with exon h | 2.45 | LnCap, MB-MDA-231, CALU6 |
| 320303 | AL079289 | Hs.137154 | H sapiens mRNA full length insert cDNA c | 2.45 | BT474, MB-MDA-231, MB-MDA-453 |
| 318359 | AI097439 | Hs.135548 | ESTs | 2.45 | NCI-H460, MB-MDA-453, NCI-H345 |
| 314384 | AA535840 | Hs.162203 | ESTs; Weakly similar to altematively sp | 2.45 | OVCA-R, PC3, EB |
| 326763 | | | CH.20_hs gi\|6598307 | 2.45 | NCI-H69, NCI-H345, RPWE-2 |
| 319900 | AW408392 | | EST cluster (not in UniGene) | 2.45 | Caco2, NCI-H460, NCI-H23 |
| 314451 | AA586368 | Hs.190232 | ESTs | 2.45 | PRSC_con, NCI-H345, MB-MDA-231 |
| 300641 | AW237699 | Hs.118346 | ESTs | 2.44 | NCI-H345, PRSC_log, PRSC_con |
| 324368 | AW299374 | | EST cluster (not in UniGene) | 2.44 | PC3, DU145, OVCA-R |
| 336510 | | | CH22_FGENES.834_5 | 2.44 | NCI-H69, RPWE-2, PRSC_con |
| 326876 | | | CH.20_hs gi\|6682507 | 2.44 | NCI-H23, NCI-H460, NCI-H520 |
| 307753 | AI340509 | Hs.182426 | ribosomal protein S2 | 2.44 | NCI-H23, NCI-H460, Caco2 |
| 317071 | M78728 | Hs.132694 | ESTs | 2.44 | NCI-H345, NCI-H69, RPWE-2 |
| 313877 | AA767869 | Hs.250113 | ESTs; Moderately similar to thyroid horm | 2.44 | DU145, LnCap, CALU6 |
| | | | component TRAP150 [H. sapiens] | | |
| 315974 | AW029203 | Hs.191952 | ESTs | 2.43 | EB, DU145, OVCA-R |
| 322970 | AI885052 | Hs.142287 | ESTs; Weakly similar to !!!! ALU CLASS F | 2.43 | NCI-H345, RPWE-2, EB |
| 317733 | AI028257 | Hs.132317 | ESTs | 2.43 | CALU6, RPWE-2, OVCA-R |
| 313599 | AA748749 | Hs.136742 | ESTs | 2.42 | NCI-H460, NCI-358, NCI-H520 |
| 323014 | AA305198 | | EST cluster (not in UniGene) | 2.42 | PRSC_con, NCI-H460, RPWE-2 |
| 324980 | AA969121 | Hs.254296 | ESTs | 2.41 | MCF7, OVCA-R, PC3 |
| 301326 | AA883831 | Hs.252924 | ESTs | 2.41 | PRSC_con, PRSC_log, RPWE-2 |
| 308695 | AI763350 | | EST singleton (not in UniGene) with exon | 2.41 | RPWE-2, NCI-H69, NCI-H345 |
| 330166 | | | CH.02_p2 gi\|6648220 | 2.41 | CALU6, DU145, A549 |
| 317552 | AW451400 | Hs.127019 | ESTs | 2.41 | NCI-358, NCI-358, NCI-H23 |
| 320572 | AI929508 | Hs.159590 | lymphocyte antigen 6 complex; locus H | 2.41 | CALU6, HT29, A549 |
| 315618 | AI287341 | Hs.154029 | ESTs; Weakly similar to TRANSCRIPTION FA | 2.41 | OVCA-R, Caco2, MB-MDA-231 |
| 331610 | N91109 | Hs.54681 | ESTs | 2.41 | NCI-H23, NCI-H520, NCI-358 |
| 311731 | AW393528 | Hs.246875 | ESTs | 2.41 | NCI-H69, NCI-H345, PRSC_con |
| 318571 | Z43383 | Hs.8053 | ESTs | 2.4 | NCI-358, NCI-H23, NCI-H520 |
| 334958 | | | CH22_FGENES.465_27 | 2.4 | DU145, PRSC_con, RPWE-2 |
| 323570 | AL038623 | Hs.208752 | ESTs; Weakly similar to !!!! ALU SUBFAMI | 2.4 | OVCA-R, EB, BT474 |
| 301685 | W67730 | | EST cluster (not in UniGene) with exon h | 2.4 | MB-MDA-231, NCI-H345, EB |
| 303849 | AW163324 | | EST cluster (not in UniGene) with exon h | 2.4 | RPWE-2, PRSC_log, NCI-H345 |
| 325702 | | | CH.14_hs gi\|5867028 | 2.4 | NCI-H23, NCI-H460, NCI-H520 |
| 313074 | N48261 | Hs.127171 | ESTs | 2.4 | MB-MDA-231, RPWE-2, PRSC_log |
| 308994 | AI880051 | | EST singleton (not in UniGene) with exon | 2.4 | RPWE-2, EB, PRSC_con |
| 330338 | | | CH.08_p2 gi\|5457162 | 2.4 | DU145, EB, LnCap |
| 327274 | | | CH.01_hs gi\|5867470 | 2.4 | OVCA-R, DU145, MB-MDA-231 |
| 325953 | | | CH.16_hs gi\|5867140 | 2.4 | MB-MDA-453, MB-MDA-435s, MCF7 |
| 333281 | | | CH22_FGENES.128_7 | 2.4 | NCI-H23, HT29, DU145 |
| 314778 | AW079559 | Hs.152258 | ESTs | 2.39 | EB, CALU6, Caco2 |
| 317005 | AI800251 | Hs.197773 | ESTs | 2.38 | MB-MDA-231, BT474, HT29 |
| 334257 | | | CH22_FGENES.367_5 | 2.38 | HT29, NCI-358, MB-MDA-231 |
| 324783 | AA640770 | | EST cluster (not in UniGene) | 2.38 | EB, OVCA-R, MB-MDA-453 |
| 300949 | AA534325 | Hs.162183 | ESTs | 2.38 | NCI-H69, NCI-H345, PRSC_log |
| 314957 | AW029274 | Hs.208368 | ESTs; Moderately similar to !!!! ALU SUB | 2.38 | LnCap, DU145, DU145 |
| 324350 | AW292501 | Hs.157174 | ESTs; Weakly similar to similar to SH3-b | 2.38 | HT29, NCI-H23, NCI-H23 |
| 338235 | | | CH22_EM:AC005500.GENSCAN.260-16 | 2.38 | NCI-H69, NCI-H460, NCI-H23 |
| 300937 | AW297302 | Hs.255631 | ESTs | 2.38 | PRSC_log, PRSC_con, PRSC_con |
| 317439 | AW451327 | Hs.170623 | ESTs | 2.38 | A549, DU145, EB |
| 324745 | AI742120 | Hs.116506 | ESTs; Weakly similar to !!!! ALU SUBFAMI | 2.38 | NCI-358, NCI-H460, BT474 |
| 338306 | | | CH22_EM:AC005500.GENSCAN.302-2 | 2.38 | NCI-H69, PRSC_con, PRSC_log |
| 318765 | Z42071 | Hs.23961 | ESTs | 2.38 | LnCap, NCI-H23, NCI-H520 |
| 310254 | AI239811 | Hs.157491 | ESTs | 2.37 | OVCA-R, DU145, EB |
| 305116 | AA649244 | | EST singleton (not in UniGene) with exon | 2.37 | CALU6, MB-MDA-435s, MB-MDA-453 |
| 324016 | AL045285 | Hs.245849 | ESTs; Moderately similar to !!!! ALU SUB | 2.37 | EB, DU145, OVCA-R |
| 322774 | AA131111 | | EST cluster (not in UniGene) | 2.37 | OVCA-R, EB, A549 |
| 335745 | | | CH22_FGENES.601_16 | 2.37 | PRSC_log, PRSC_con, NCI-H69 |
| 300972 | AI979100 | Hs.211518 | ESTs | 2.37 | NCI-H69, NCI-H345, PRSC_log |
| 338809 | | | CH22_EMAC005500.GENSCAN.531-10 | 2.37 | NCI-H23, NCI-H69, NCI-H520 |
| 316983 | AI480204 | Hs.177131 | ESTs | 2.37 | NCI-H345, PRSC_con, PRSC_log |
| 321308 | AI247480 | Hs.117029 | ESTs | 2.37 | BT474, NCI-H69, HT29 |
| 323578 | AA299492 | Hs.168166 | ESTs | 2.37 | LnCap, EB, MB-MDA-453 |
| 335747 | | | CH22_FGENES.601_20 | 2.36 | NCI-H69, LnCap, PRSC_con |
| 322362 | AF039697 | | EST cluster (not in UniGene) | 2.36 | DU145, PRSC_con, NCI-H345 |
| 314430 | N76302 | Hs.78110 | ESTs; Weakly similar to F17A9.2 [C. elega | 2.36 | DU145, MB-MDA-453, CALU6 |
| 304831 | AA586422 | | EST singleton (not in UniGene) with exon | 2.36 | NCI-H23, NCI-H460, CALU6 |
| 337432 | | | CH22_FGENES.765-1 | 2.36 | MB-MDA-231, BT474, HT29 |
| 305984 | AA887654 | | EST singleton (not in UniGene) with exon | 2.36 | DU145, HT29, CALU6 |
| 313486 | AW134523 | Hs.247186 | ESTs | 2.36 | DU145, A549, CALU6 |
| 309028 | AI889109 | Hs.212032 | EST | 2.36 | NCI-358, NCI-H520, NCI-H23 |
| 318292 | AI679966 | Hs.150603 | ESTs | 2.35 | NCI-H460, Caco2, NCI-H23 |
| 334198 | | | CH22_FGENES.354_4 | 2.35 | NCI-H69, PRSC_log, PRSC_con |
| 314458 | AI217440 | Hs.143873 | ESTs | 2.35 | Caco2, A549, PC3 |
| 333346 | | | CH22_FGENES.139_15 | 2.35 | CALU6, DU145, LnCap |
| 325408 | | | CH.12_hs gi\|5866921 | 2.35 | NCI-H460, NCI-H520, NCI-H23 |
| 313758 | AA076743 | Hs.129770 | ESTs | 2.35 | NCI-H23, MB-MDA-435s, NCI-H345 |
| 309825 | AW293701 | | EST singleton (not in UniGene) with exon | 2.35 | NCI-H460, NCI-H23, NCI-H520 |
| 303536 | R55497 | Hs.183941 | ESTs; Moderately similar to H beta 58 ho | 2.35 | DU145, CALU6, NCI-H520 |
| 331534 | N51583 | Hs.133756 | EST | 2.35 | NCI-H23, NCI-H520, NCI-358 |
| 325164 | T16981 | Hs.21963 | ESTs | 2.34 | NCI-H345, PRSC_log, NCI-H460 |
| 327710 | | | CH.04_hs gi\|5867860 | 2.34 | BT474, MB-MDA-231, NCI-H345 |
| 306351 | AA961356 | | EST singleton (not in UniGene) with exon | 2.34 | BT474, MB-MDA-231, MB-MDA-435s |
| 304968 | AA614308 | | EST singleton (not in UniGene) with exon | 2.34 | CALU6, HT29, MB-MDA-453 |
| 334015 | | | CH22_FGENES.313_7 | 2.34 | HT29, MB-MDA-231, BT474 |
| 318315 | AI091370 | Hs.134852 | ESTs | 2.33 | CALU6, NCI-H520, DU145 |
| 306809 | AI057134 | | EST singleton (not in UniGene) with exon | 2.33 | PC3, DU145, EB |
| 337697 | | | CH22_EM:AC000097.GENSCAN.86-1 | 2.33 | RPWE-2, PRSC_log, NCI-H345 |
| 329630 | | | CH.11_p2 gi\|6729060 | 2.33 | NCI-H520, NCI-H23, NCI-H460 |
| 326577 | | | CH.19_hs gi\|5867317 | 2.33 | NCI-H460, NCI-358, NCI-H23 |
| 333428 | | | CH22_FGENES.149_1 | 2.33 | NCI-H345, PRSC_con, RPWE-2 |
| 301080 | AI479391 | Hs.155405 | ESTs; Weakly similar to !!!! ALU SUBFAMI | 2.33 | OVCA-R, MCF7, MCF7 |
| 324829 | AA714311 | | EST cluster (not in UniGene) | 2.33 | NCI-H460, NCI-358, NCI-H23 |
| 302776 | AJ133798 | | EST cluster (not in UniGene) with exon h | 2.32 | NCI-H23, NCI-H460, NCI-H520 |
| 325801 | | | CH.14_hs gi\|6552451 | 2.32 | PRSC_log, MCF7, NCI-H23 |
| 332122 | AA609698 | Hs.112389 | ESTs | 2.32 | DU145, HT29, PC3 |
| 314167 | AA243633 | Hs.208983 | ESTs | 2.32 | DU145, MCF7, PC3 |
| 324023 | AA669615 | Hs.214226 | ESTs | 2.31 | DU145, NCI-H345, EB |
| 320503 | NM_00589 | | EST cluster (not in UniGene) | 2.31 | A549, OVCA-R, PC3 |
| 312217 | T98289 | | EST cluster (not in UniGene) | 2.31 | NCI-H23, Caco2, NCI-H69 |
| 321304 | AA078293 | | EST cluster (not in UniGene) | 2.31 | DU145, OVCA-R, EB |
| 323517 | AA527359 | Hs.154366 | ESTs | 2.31 | NCI-H345, DU145, EB |
| 336455 | | | CH22_FGENES.829_13 | 2.31 | NCI-H345, PRSC_con, RPWE-2 |
| 313352 | AW292127 | Hs.144758 | ESTs | 2.31 | MCF7, DU145, OVCA-R |
| 331457 | H93135 | Hs.41840 | ESTs | 2.31 | Caco2, NCI-H460, NCI-H23 |
| 333054 | | | CH22_FGENES.73_8 | 2.31 | NCI-H69, NCI-358, NCI-H23 |
| 308598 | AI719237 | | EST singleton (not in UniGene) with exon | 2.31 | OVCA-R, CALU6, Caco2 |
| 327059 | | | CH.21_hs gi\|6531965 | 2.3 | NCI-H460, LnCap, LnCap |
| 334120 | | | CH22_FGENES.333_1 | 2.3 | NCI-H69, RPWE-2, MB-MDA-435s |
| 324154 | AI457449 | Hs.192817 | ESTs | 2.3 | NCI-H460, MB-MDA-453, NCI-358 |
| 326509 | | | CH.19_hs gi\|6682496 | 2.3 | NCI-H345, CALU6, OVCA-R |
| 316855 | AW291384 | Hs.254974 | ESTs | 2.3 | NCI-H345, NCI-H460, BT474 |
| 337918 | | | CH22_EM:AC005500.GENSCAN.66-4 | 2.3 | RPWE-2, NCI-H345, PRSC_log |
| 317471 | AI825351 | Hs.144084 | ESTs | 2.29 | HT29, OVCA-R, DU145 |
| 331023 | N32599 | Hs.5856 | ESTs | 2.29 | OVCA-R, LnCap, A549 |
| 332231 | N48008 | Hs.102629 | EST | 2.29 | CALU6, DU145, EB |
| 309912 | AW339671 | | EST singleton (not in UniGene) with exon | 2.29 | MB-MDA-435s, PRSC_con, NCI-358 |
| 316427 | AI241019 | Hs.145644 | ESTs | 2.29 | Caco2, HT29, EB |
| 313329 | AW293704 | Hs.122658 | ESTs | 2.29 | OVCA-R, DU145, Caco2 |
| 335019 | | | CH22_FGENES.474_7 | 2.29 | HT29, CALU6, MB-MDA-231 |
| 324394 | F20654 | Hs.152128 | ESTs; Moderately similar to !!!! ALU SUB | 2.29 | NCI-H345, MB-MDA-231, RPWE-2 |
| 339357 | | | CH22_BA354\|12.GENSCAN.31-2 | 2.29 | NCI-H69, OVCA-R, BT474 |
| 322128 | AI346033 | | EST cluster (not in UniGene) | 2.28 | NCI-H23, NCI-H520, NCI-H460 |
| 301310 | AI239457 | Hs.130794 | ESTs | 2.28 | OVCA-R, DU145, MB-MDA-231 |
| 300623 | AI929130 | Hs.118261 | ESTs; Moderately similar to finger prote | 2.28 | BT474, RPWE-2, PRSC_con |
| 323409 | AL135534 | | EST cluster (not in UniGene) | 2.27 | NCI-H345, NCI-358, Caco2 |
| 308406 | AI634885 | | EST singleton (not in UniGene) with exon | 2.27 | OVCA-R, EB, HT29 |
| 322518 | AI133446 | | EST cluster (not in UniGene) | 2.27 | DU145, MB-MDA-435s, OVCA-R |
| 338381 | | | CH22_EM:AC005500.GENSCAN.330-10 | 2.27 | NCI-H69, PRSC_con, PRSC_log |
| 316003 | AA704584 | Hs.119993 | ESTs | 2.27 | NCI-358, NCI-H520, NCI-H23 |
| 307090 | AI161024 | | EST singleton (not in UniGene) with exon | 2.27 | NCI-H345, DU145, RPWE-2 |
| 300356 | AA758411 | Hs.121335 | ESTs | 2.27 | LnCap, NCI-H460, Caco2 |
| 331887 | AA431328 | Hs.98660 | ESTs | 2.27 | NCI-358, NCI-H520, CALU6 |
| 330951 | H02566 | Hs.191268 | H sapiens mRNA; cDNA DKFZp434N174 (from | 2.27 | OVCA-R, BT474, BT474 |
| 305547 | AA773111 | | EST singleton (not in UniGene) with exon | 2.27 | LnCap, DU145, BT474 |
| 312457 | AA776743 | Hs.191589 | ESTs | 2.26 | NCI-H345, RPWE-2, PRSC_con |
| 333929 | | | CH22_FGENES.300_2 | 2.26 | HT29, CALU6, EB |
| 319845 | AA649011 | Hs.187902 | ESTs | 2.26 | LnCap, DU145, MCF7 |
| 306739 | AI028393 | | EST singleton (not in UniGene) with exon | 2.26 | MB-MDA-435s, NCI-358, CALU6 |
| 306919 | AI096832 | | EST singleton (not in UniGene) with exon | 2.26 | HT29, BT474, PC3 |
| 333312 | | | CH22_FGENES.138_4 | 2.26 | OVCA-R, DU145, PC3 |
| 334955 | | | CH22_FGENES.465_24 | 2.25 | RPWE-2, PRSC_con, NCI-H345 |
| 312295 | AA578233 | Hs.173863 | ESTs | 2.25 | OVCA-R, DU145, NCI-H345 |
| 307643 | AI302124 | | EST singleton (not in UniGene) with exon | 2.25 | CALU6, CALU6, OVCA-R |
| 324252 | AA421989 | | EST cluster (not in UniGene) | 2.25 | OVCA-R, EB, A549 |
| 309767 | AW271805 | | EST singleton (not in UniGene) with exon | 2.25 | DU145, NCI-H460, CALU6 |
| 311492 | AW410240 | Hs.4437 | ribosomal protein L28 | 2.25 | NCI-H69, NCI-H460, NCI-H520 |
| 312260 | H05392 | Hs.230597 | EST | 2.25 | Caco2, EB, DU145 |
| 327125 | | | CH.21_hs gi\|6531971 | 2.25 | HT29, NCI-358, BT474 |
| 316919 | AA845382 | Hs.204520 | ESTs | 2.24 | NCI-H23, NCI-H345, NCI-H520 |
| 316361 | AI433833 | Hs.164159 | ESTs; Weakly similar to !!!! ALU SUBFAMI | 2.24 | DU145, EB, PC3 |
| 315772 | AW515373 | Hs.158893 | ESTs | 2.24 | OVCA-R, EB, LnCap |
| 320236 | H03688 | | EST cluster (not in UniGene) | 2.24 | NCI-358, DU145, NCI-H23 |
| 315444 | AW138821 | Hs.221737 | ESTs | 2.24 | NCI-358, CALU6, PRSC_con |
| 333903 | | | CH22_FGENES.294_1 | 2.24 | MB-MDA-231, BT474, A549 |
| 335234 | | | CH22_FGENES.515_3 | 2.24 | NCI-H69, PRSC_con, PRSC_log |
| 333727 | | | CH22_FGENES.256_1 | 2.23 | MB-MDA-231, NCI-H69, BT474 |
| 332002 | AA482009 | Hs.105104 | ESTs | 2.23 | EB, NCI-H520, HT29 |
| 329611 | | | CH.10_p2 gi\|3962478 | 2.23 | BT474, HT29, MB-MDA-231 |
| 310559 | AI783594 | Hs.155718 | ESTs | 2.22 | BT474, MCF7, MB-MDA-231 |
| 327315 | | | CH.01_hs gi\|5867508 | 2.22 | NCI-H69, EB, EB |
| 323170 | U83527 | | EST cluster (not in UniGene) | 2.22 | EB, DU145, LnCap |
| 331522 | N49309 | Hs.117012 | ESTs | 2.22 | A549, LnCap, DU145 |
| 313261 | AA730472 | Hs.142805 | ESTs | 2.22 | OVCA-R, PC3, LnCap |
| 312740 | R97191 | Hs.134106 | ESTs | 2.22 | BT474, MCF7, OVCA-R |
| 325055 | Z44631 | Hs.21658 | ESTs | 2.22 | MB-MDA-453, DU145, CALU6 |
| 337895 | | | CH22_EM:AC005500.GENSCAN.56-2 | 2.22 | NCI-H345, PRSC_log, PRSC_con |
| 307140 | AI185762 | | EST singleton (not in UniGene) with exon | 2.22 | NCI-H520, NCI-H460, EB |
| 321643 | W76005 | Hs.32094 | ESTs | 2.21 | EB, NCI-H345, PRSC_con |
| 302683 | X85153 | | EST cluster (not in UniGene) with exon h | 2.21 | BT474, MB-MDA-231, MCF7 |
| 322644 | AA340904 | | EST cluster (not in UniGene) | 2.21 | NCI-H460, NCI-H23, NCI-H520 |
| 330415 | D83777 | Hs.75137 | KIAA0193 gene product | 2.21 | CALU6, A549, Caco2 |
| 302334 | AF120491 | | EST cluster (not in UniGene) with exon h | 2.21 | NCI-H69, NCI-H345, PC3 |
| 326710 | | | CH.20_hs gi\|5867593 | 2.21 | NCI-H520, NCI-358, NCI-H23 |
| 323561 | AA825426 | Hs.238832 | ESTs; Weakly similar to !!!! ALU SUBFAMI | 2.21 | NCI-H345, DU145, NCI-H69 |
| 337706 | | | CH22_EM:AC000097.GENSCAN.87-11 | 2.21 | MB-MDA-435s, NCI-358, NCI-H520 |
| 339309 | | | CH22_BA354I12.GENSCAN.22-7 | 2.21 | BT474, HT29, PC3 |
| 330436 | HG2724-H | | Oncogene Tls/Chop, Fusion Activated | 2.21 | PRSC_con, NCI-H69, Caco2 |
| 312360 | AI922972 | Hs.196073 | ESTs | 2.21 | OVCA-R, MB-MDA-435s, DU145 |
| 301855 | AF053356 | | multiple UniGene matches | 2.2 | NCI-H69, HT29, NCI-H23 |
| 331192 | T55182 | Hs.152571 | ESTs; Highly similar to IGF-II mRNA-bind | 2.2 | OVCA-R, PC3, CALU6 |
| 315872 | AW051819 | Hs.204516 | ESTs | 2.2 | LnCap, OVCA-R, EB |
| 337904 | | | CH22_EM:AC005500.GENSCAN.56-17 | 2.2 | OVCA-R, LnCap, EB |
| 308258 | AI565612 | | EST singleton (not in UniGene) with exon | 2.2 | DU145, MB-MDA-231, CALU6 |
| 320965 | H18166 | | EST cluster (not in UniGene) | 2.2 | DU145, EB, LnCap |
| 333910 | | | CH22_FGENES.295_3 | 2.2 | DU145, MB-MDA-231, EB |
| 300707 | AA080921 | | EST cluster (not in UniGene) with exon h | 2.2 | BT474, MCF7, HT29 |
| 336011 | | | CH22_FGENES.668_9 | 2.19 | NCI-H460, BT474, NCI-H345 |
| 325712 | | | CH.14_hs gi\|6682473 | 2.19 | NCI-H460, NCI-H23, NCI-358 |
| 322738 | AF201832 | | EST cluster (not in UniGene) | 2.19 | PC3, RPWE-2, PRSC_con |
| 335339 | | | CH22_FGENES.535_16 | 2.19 | HT29, PRSC_log, MCF7 |
| 320733 | AA738436 | Hs.134407 | ESTs | 2.19 | DU145, EB, Caco2 |
| 319412 | AA679426 | Hs.187505 | ESTs | 2.19 | NCI-H345, PRSC_log, PRSC_con |
| 337132 | | | CH22_FGENES.526-3 | 2.19 | NCI-H69, NCI-H345, PRSC_con |
| 301544 | AI951651 | Hs.224290 | ESTs | 2.19 | PRSC_con, MB-MDA-231, NCI-H23 |
| 325285 | | | CH.11_hs gi\|5866903 | 2.18 | PRSC_con, PRSC_log, MB-MDA-231 |
| 338280 | | | CH22_EM:AC005500.GENSCAN.290-11 | 2.18 | PC3, NCI-358, HT29 |
| 311421 | AI701635 | Hs.207077 | ESTs | 2.18 | RPWE-2, NCI-H345, NCI-358 |
| 330638 | X89576 | Hs.159581 | matrix metalloproteinase 17 (membrane-in | 2.18 | HT29, MB-MDA-435s, MB-MDA-453 |
| 326603 | | | CH.20_hs gi\|6056312 | 2.18 | CALU6, DU145, HT29 |
| 319055 | AA412305 | | EST cluster (not in UniGene) | 2.18 | A549, OVCA-R, MB-MDA-435s |
| 335451 | | | CH22_FGENES.562_9 | 2.18 | DU145, LnCap, CALU6 |
| 317989 | AI203009 | Hs.130664 | ESTs | 2.18 | NCI-H345, NCI-H69, NCI-H520 |
| 322024 | AA334384 | | EST cluster (not in UniGene) | 2.18 | Caco2, PC3, NCI-H520 |
| 300734 | AW205197 | Hs.240951 | ESTs | 2.18 | NCI-358, A549, EB |
| 304022 | T02990 | | EST singleton (not in UniGene) with exon | 2.18 | NCI-H23, NCI-358, NCI-H460 |
| 330082 | | | CH.19_p2 gi\|6015314 | 2.18 | NCI-H23, Caco2, Caco2 |
| 312516 | AA363245 | Hs.189831 | ESTs | 2.18 | BT474, HT29, MB-MDA-231 |
| 333932 | | | CH22_FGENES.300_5 | 2.17 | PC3, Caco2, EB |
| 308115 | AI479071 | | EST singleton (not in UniGene) with exon | 2.17 | BT474, OVCA-R, OVCA-R |
| 320184 | U91510 | Hs.123036 | CD39-like 1 | 2.17 | NCI-H520, NCI-358, NCI-H23 |
| 324432 | AA464510 | | EST cluster (not in UniGene) | 2.17 | CALU6, RPWE-2, HT29 |
| 320882 | AI832098 | | EST cluster (not in UniGene) | 2.17 | OVCA-R, PC3, BT474 |
| 312251 | H03952 | | EST cluster (not in UniGene) | 2.17 | NCI-H460, NCI-H23, NCI-358 |
| 315049 | AW340486 | Hs.121210 | ESTs | 2.17 | NCI-H520, NCI-358, NCI-H23 |
| 305018 | AA627127 | | EST singleton (not in UniGene) with exon | 2.17 | MB-MDA-231, MB-MDA-453, EB |
| 303807 | AI792785 | Hs.130434 | ESTs | 2.16 | NCI-H345, PRSC_con, PRSC_log |
| 317792 | AI653389 | Hs.196121 | ESTs | 2.16 | NCI-H345, PRSC_con, LnCap |
| 321668 | AA872730 | Hs.125229 | ESTs | 2.16 | OVCA-R, PC3, MCF7 |
| 328863 | | | CH.07_hs gi\|6381929 | 2.16 | PRSC_con, NCI-H345, NCI-H460 |
| 319373 | R00371 | | EST cluster (not in UniGene) | 2.16 | PRSC_con, RPWE-2, NCI-H345 |
| 320069 | T86541 | Hs.189732 | ESTs | 2.16 | NCI-H23, NCI-358, NCI-H345 |
| 320235 | AF064090 | Hs.129708 | tumor necrosis factor (ligand) superfami | 2.16 | NCI-H23, NCI-8460, NCI-H520 |
| 338880 | | | CH22_DJ32I10.GENSCAN.6-2 | 2.16 | BT474, MCF7, OVCA-R |
| 318314 | AI091349 | Hs.161133 | ESTs | 2.16 | NCI-H23, NCI-H520, NCI-H460 |
| 332696 | D86973 | Hs.75354 | GCN1 (general control of amino-acid synt | 2.16 | A549, PC3, DU145 |
| 331352 | AA406133 | Hs.7482 | KIAA0682 gene product | 2.16 | PC3, EB, MB-MDA-231 |
| 339019 | | | CH22_DA59H18.GENSCAN.21-15 | 2.15 | LnCap, EB, OVCA-R |
| 306975 | AI127042 | | EST singleton (not in UniGene) with exon | 2.15 | MB-MDA-435s, NCI-H520, NCI-358 |
| 318069 | AI024557 | Hs.131540 | ESTs | 2.15 | Caco2, Caco2, BT474 |
| 312997 | AW205686 | Hs.135130 | ESTs | 2.15 | NCI-H460, NCI-H23, NCI-358 |
| 331372 | AA433935 | Hs.55044 | DKFZP586H2123 protein | 2.15 | PRSC_con, HT29, CALU6 |
| 335049 | | | CH22_FGENES.481_5 | 2.15 | NCI-H69, NCI-H345, PRSC_log |
| 324280 | AA429772 | Hs.191610 | ESTs | 2.15 | MB-MDA-453, MB-MDA-435s, MCF7 |
| 330363 | | | CH.X_p2 gi\|3126882 | 2.15 | NCI-H23, NCI-H460, NCI-358 |
| 322896 | AW470296 | Hs.144830 | ESTs | 2.15 | HT29, CALU6, EB |
| 321981 | AA948204 | Hs.127361 | ESTs | 2.15 | MB-MDA-231, DU145, HT29 |
| 333294 | | | CH22_FGENES.130_6 | 2.14 | EB, DU145, MB-MDA-453 |
| 330170 | | | CH.02_p2 gi\|6648220 | 2.14 | HT29, MB-MDA-453, PC3 |
| 312973 | AI123346 | Hs.135241 | ESTs | 2.14 | LnCap, DU145, EB |
| 311104 | AI627352 | Hs.201449 | ESTs | 2.14 | NCI-H520, NCI-H23, LnCap |
| 325086 | T10019 | Hs.4194 | ESTs | 2.14 | NCI-H460, NCI-H23, NCI-358 |
| 317182 | AW183524 | Hs.192298 | ESTs | 2.14 | HT29, BT474, MB-MDA-435s |
| 323644 | AA310711 | Hs.124340 | ESTs | 2.14 | RPWE-2, PRSC_con, PRSC_log |
| 308092 | AI474896 | | EST singleton (not in UniGene) with exon | 2.14 | BT474, MCF7, MB-MDA-231 |
| 322265 | AF086244 | | EST cluster (not in UniGene) | 2.14 | NCI-H345, RPWE-2, PRSC_con |
| 303521 | AA746272 | | EST cluster (not in UniGene) with exon h | 2.14 | DU145, MB-MDA-453, EB |
| 312102 | AW439340 | Hs.189720 | ESTs | 2.14 | NCI-H23, NCI-H460, MB-MDA-435s |
| 316559 | AI249468 | Hs.228251 | EST | 2.14 | NCI-H460, NCI-358, NCI-H23 |
| 338486 | | | CH22_EM:AC005500.GENSCAN.382-8 | 2.14 | NCI-H520, NCI-H23, NCI-H69 |
| 301302 | AI825444 | Hs.210956 | ESTs | 2.14 | BT474, HT29, MB-MDA-231 |
| 310591 | AI650372 | Hs.195979 | ESTs | 2.14 | CALU6, CALU6, Caco2 |
| 316231 | AA732301 | | EST cluster (not in UniGene) | 2.14 | NCI-H23, NCI-H520, NCI-358 |
| 326559 | | | CH.19_hs gi\|5867310 | 2.14 | DU145, NCI-H460, NCI-H23 |
| 324062 | AA525291 | Hs.204099 | ESTs; Weakly similar to !!!! ALU SUBFAMI | 2.13 | OVCA-R, DU145, EB |
| 323844 | AI811303 | Hs.143490 | ESTs | 2.13 | MB-MDA-453, MCF7, MB-MDA-435s |
| 333895 | | | CH22_FGENES.293_2 | 2.13 | CALU6, LnCap, DU145 |
| 308264 | AI567114 | Hs.171454 | EST | 2.13 | DU145, CALU6, MB-MDA-453 |
| 306081 | AA908472 | | EST singleton (not in UniGene) with exon | 2.13 | HT29, 6T474, MB-MDA-231 |
| 333101 | | | CH22_FGENES.79_6 | 2.13 | NCI-H345, NCI-H69, PRSC_log |
| 328544 | | | CH.07_hs gi\|5868486 | 2.13 | NCI-H23, NCI-H69, PRSC_log |
| 333355 | | | CH22_.FGENES.141_6 | 2.13 | DU145, EB, CALU6 |
| 323397 | AI524519 | Hs.239699 | ESTs | 2.13 | EB, NCI-H460, NCI-H345 |
| 305697 | AA814956 | | EST singleton (not in UniGene) with exon | 2.13 | NCI-H520, NCI-H460, NCI-358 |
| 327809 | | | CH.05_hs gi\|5867968 | 2.13 | HT29, PC3, OVCA-R |
| 325092 | T10115 | Hs.92423 | ESTs | 2.13 | HT29, NCI-358, MB-MDA-231 |
| 322299 | AI971935 | Hs.252784 | ESTs | 2.13 | PRSC_con, DU145, DU145 |
| 312145 | AA029526 | Hs.126706 | ESTs | 2.12 | OVCA-R, A549, MB-MDA-435s |
| 323704 | AA319421 | Hs.193577 | ESTs | 2.12 | Caco2, LnCap, OVCA-R |
| 328971 | | | CH.08_hs gi\|6478806 | 2.12 | NCI-358, NCI-H23, NCI-H520 |
| 325338 | | | CH.11_hs gi\|5866883 | 2.12 | LnCap, NCI-H69, NCI-H345 |
| 331332 | AA282554 | Hs.89034 | ESTs | 2.12 | NCI-H520, NCI-H23, Caco2 |
| 327159 | | | CH.01_hs gi\|5867550 | 2.12 | EB, DU145, PC3 |
| 335180 | | | CH22_FGENES.505_2 | 2.12 | LnCap, NCI-H69, A549 |
| 338062 | | | CH22_EM:AC005500.GENSCAN.162-3 | 2.12 | PRSC_con, PRSC_log, NCI-H69 |
| 318350 | AI636018 | Hs.135538 | ESTs | 2.12 | EB, HT29, DU145 |
| 312070 | AW293140 | Hs.108790 | ESTs | 2.11 | Caco2, NCI-H23, A549 |
| 328314 | | | CH.07_hs gi\|5868371 | 2.11 | HT29, NCI-H23, NCI-H460 |
| 315869 | AI033547 | Hs.132826 | ESTs | 2.11 | BT474, CALU6, MCF7 |
| 339246 | | | CH22_BA354\|12.GENSCAN.5-9 | 2.11 | CALU6, CALU6, BT474 |
| 329921 | | | CH.16_p2 gi\|6165205 | 2.11 | BT474, MB-MDA-231, HT29 |
| 324981 | Z25333 | Hs.4947 | ESTs | 2.11 | A549, NCI-H460, NCI-H520 |
| 331291 | AAI59323 | Hs.109929 | ESTs | 2.11 | NCI-H345, A549, PRSC_con |
| 332729 | AA058907 | Hs.83190 | fatty acid synthase | 2.11 | NCI-358, LnCap, MB-MDA-453 |
| 325448 | | | CH.12_hs gi\|5866941 | 2.11 | DU145, MCF7, CALU6 |
| 314929 | AW188286 | Hs.143612 | ESTs | 2.1 | EB, BT474, MB-MDA-231 |
| 301063 | AI057634 | Hs.124596 | ESTs | 2.1 | NCI-H23, NCI-H460, BT474 |
| 301952 | A5029016 | Hs.117333 | KIAA1093 protein | 2.1 | OVCA-R, A549, CALU6 |
| 326309 | | | CH.17_hs gi\|5867277 | 2.1 | MB-MDA-435s, NCI-H69, MB-MDA-453 |
| 315406 | AI823453 | Hs.146625 | ESTs | 2.1 | OVCA-R, DU145, EB |
| 302376 | AB007867 | Hs.200480 | KIAA0407 protein | 2.1 | OVCA-R, Caco2, HT29 |
| 312181 | AA417281 | Hs.191595 | ESTs | 2.1 | OVCA-R, A549, DU145 |
| 334254 | | | CH22_FGENES.366_4 | 2.1 | LnCap, OVCA-R, DU145 |
| 318073 | AW167087 | Hs.131562 | ESTs | 2.1 | A549, CALU6, EB |
| 304724 | AA569881 | Hs.65114 | keratin 18 | 2.1 | NCI-H23, NCI-H520, NCI-H460 |
| 332359 | W87704 | Hs.211558 | ESTs | 2.1 | MB-MDA-435s, PRSC_con, NCI-H460 |
| 331884 | AA431302 | Hs.98721 | EST: Weakly similar to N-copine [H. sapie | 2.1 | NCI-H345, MB-MDA-231, PRSC_con |
| 308226 | AI559106 | Hs.181165 | eukaryotic translation elongation factor | 2.1 | EB, CALU6, OVCA-R |
| 324279 | AA501412 | Hs.191688 | ESTs; Weakly similar to Pro-Pol-dUTPase | 2.09 | OVCA-R, LnCap, PC3 |
| 337203 | | | CH22_.FGENES.591-3 | 2.09 | NCI-H69, NCI-H345, MB-MDA-231 |
| 322346 | AA227618 | Hs.10882 | HMG-box containing protein 1 | 2.09 | HT29, BT474, MB-MDA-231 |
| 304470 | AA426654 | Hs.195188 | glyceraldehyde-3-phosphate dehydrogenase | 2.09 | NCI-H23, CALU6, NCI-H520 |
| 325977 | | | CH.16_hs gi\|6249602 | 2.09 | NCI-H23, NCI-H520, HT29 |
| 304696 | AA554758 | | EST singleton (not in UniGene) with exon | 2.09 | MB-MDA-435s, NCI-H23, BT474 |
| 317412 | AI301528 | Hs.132604 | ESTs | 2.09 | Caco2, EB, NCI-358 |
| 315570 | AI860360 | Hs.160316 | ESTs | 2.08 | PRSC_con, PRSC_log, NCI-H345 |
| 327341 | | | CH.01_hs gi\|6017016 | 2.08 | MB-MDA-231, PRSC_con, NCI-H69 |
| 327431 | | | CH.02_hs gi\|5867754 | 2.08 | NCI-H23, NCI-358, NCI-H520 |
| 314685 | AI870811 | Hs.158709 | ESTs; Weakly similar to KIAA0938 protein | 2.08 | MB-MDA-453, MCF7, OVCA-R |
| 328624 | | | CH.07_hs gi\|5868246 | 2.08 | MCF7, NCI-358, RPWE-2 |
| 303596 | AW303377 | | EST cluster (not in UniGene) with exon h | 2.08 | RPWE-2, PRSC_con, PRSC_log |
| 336717 | | | CH22_FGENES.81-1 | 2.08 | BT474, HT29, MCF7 |
| 317370 | AW204139 | Hs.174424 | ESTs; Weakly similar to p140mDia [M. musc | 2.08 | NCI-H23, NCI-H460, NCI-H69 |
| 331287 | AA149061 | Hs.172971 | ESTs | 2.08 | OVCA-R, EB, NCI-H345 |
| 304211 | N62228 | | EST singleton (not in UniGene) with exon | 2.08 | BT474, MCF7, MB-MDA-231 |
| 315613 | AW137420 | Hs.192311 | ESTs | 2.08 | PRSC_con, PRSC_log, PRSC_log |
| 325636 | | | CH.14_hs gi\|5867002 | 2.08 | NCI-358, NCI-H460, MB-MDA-453 |
| 336406 | | | CH22_FGENES.823_21 | 2.08 | HT29, EB, DU145 |
| 301714 | F06529 | | EST cluster (not in UniGene) with exon h | 2.08 | LnCap, PRSC_log, PRSC_con |
| 300496 | R45159 | Hs.221804 | ESTs | 2.08 | PRSC_con, LnCap, RPWE-2 |
| 318970 | R21114 | Hs.21383 | ESTs | 2.08 | NCI-H23, NCI-H520, NCI-H460 |
| 334115 | | | CH22_FGENES.330_115 | 2.08 | BT474, NCI-H69, HT29 |
| 308082 | AI473682 | | EST singleton (not in UniGene) with exon | 2.08 | MB-MDA-435s, NCI-H345, MB-MDA-231 |
| 308282 | AI569456 | | EST singleton (not in UniGene) with exon | 2.08 | LnCap, EB, PRSC_con |
| 313038 | AW451618 | Hs.124195 | ESTs | 2.07 | NCI-H345, PRSC_con, LnCap |
| 317974 | AW444468 | Hs.144900 | ESTs | 2.07 | NCI-358, NCI-H23, NCI-H520 |
| 324063 | AW292740 | Hs.254815 | ESTs | 2.07 | Caco2, NCI-358, NCI-H520 |
| 334759 | | | CH22_FGENES.428_8 | 2.07 | CALU6, HT29, NCI-H520 |
| 307864 | AI367417 | | EST singleton (not in UniGene) with exon | 2.07 | NCI-H460, NCI-358, NCI-H423 |
| 304356 | AA196027 | Hs.195188 | glyceraldehyde-3-phosphate dehydrogenase | 2.07 | HT29, MCF7, MB-MDA-435s |
| 303929 | AW470753 | | EST singleton (not in UniGene) with exon | 2.07 | NCI-H345, PRSC_con, RPWE-2 |
| 331857 | AA421160 | Hs.9456 | SWI/SNF related; matrix assocd; actin de | 2.07 | EB, A549, PC3 |
| 322814 | AI824495 | Hs.211038 | ESTs | 2.06 | PRSC_con, RPWE-2, Caco2 |
| 303650 | AA430709 | | EST cluster (not in UniGene) with exon h | 2.06 | RPWE-2, NCI-H345, PRSC_con |
| 333403 | | | CH22_FGENES.144_21 | 2.06 | OVCA-R, CALU6, PC3 |
| 313663 | AI953261 | Hs.169813 | ESTs | 2.06 | NCI-H345, OVCA-R, NCI-H23 |
| 338594 | | | CH22_EM:AC005500.GENSCAN.435-4 | 2.06 | DU145, LnCap, EB |
| 334676 | | | CH22_FGENES.418_29 | 2.06 | NCI-H69, PRSC_log, PRSC_con |
| 310046 | AI198032 | Hs.210356 | ESTs | 2.06 | MB-MDA-435s, NCI-H23, Caco2 |
| 309169 | AI949216 | | EST singleton (not in UniGene) with exon | 2.06 | CALU6, EB, NCI-358 |
| 329752 | | | CH.14_p2 gi\|6065777 | 2.06 | CALU6, HT29, DU145 |
| 325085 | T10001 | Hs.4188 | ESTs | 2.06 | EB, OVCA-R, MB-MDA-435s |
| 332062 | AA521016 | Hs.185375 | ESTs | 2.06 | OVCA-R, MB-MDA-453, MCF7 |
| 302074 | AA382871 | Hs.132794 | phosphate cytidylyltransferase 1; cholin | 2.06 | LnCap, EB, NCI-H69 |
| 326344 | | | CH.17_hs gi\|6525295 | 2.06 | HT29, BT474, MB-MDA-453 |
| 330855 | AA079318 | | zm98c2.s1 Stratagene colon HT29 (#937221 | 2.06 | RPWE-2, LnCap, PRSC_con |
| | | | IMAGE:545954 3′, mRNA seq | | |
| 302525 | AF024690 | Hs.248056 | G protein-coupled receptor 43 | 2.05 | NCI-358, NCI-H23, DU145 |
| 331903 | AA436673 | Hs.29417 | H sapiens mRNA; cDNA DKFZp586B0323 | 2.05 | Caco2, DU145, A549 |
| | | | (from | | |
| 316322 | AW296618 | Hs.120637 | ESTs | 2.05 | BT474, MB-MDA-453, OVCA-R |
| 321525 | H78875 | | EST cluster (not in UniGene) | 2.05 | NCI-H23, PRSC_con, NCI-H520 |
| 305071 | AA640579 | | EST singleton (not in UniGene) with exon | 2.05 | MB-MDA-231, BT474, HT29 |
| 326033 | | | CH.17_hs gi\|5867178 | 2.05 | HT29, DU145, BT474 |
| 334730 | | | CH22_FGENES.424_5 | 2.05 | BT474, EB, OVCA-R |
| 305335 | AA704235 | | EST singleton (not in UniGene) with exon | 2.05 | MCF7, OVCA-R, MB-MDA-453 |
| 320521 | N31464 | Hs.24743 | ESTs | 2.05 | MB-MDA-453, MB-MDA-231, PC3 |
| 333515 | | | CH22_FGENES.172_5 | 2.04 | NCI-H345, RPWE-2, PRSC_con |
| 311020 | AI918672 | Hs.213783 | ESTs | 2.04 | NCI-H460, NCI-H23, NCI-H520 |
| 324323 | AA393739 | | EST cluster (not in UniGene) | 2.04 | OVCA-R, PC3, LnCap |
| 305486 | AA748889 | | EST singleton (not in UniGene) with exon | 2.04 | NCI-H345, PRSC_log, CALU6 |
| 312162 | T91823 | | EST cluster (not in UniGene) | 2.04 | NCI-H520, NCI-H23, NCI-358 |
| 330980 | H28794 | Hs.6659 | ESTs | 2.04 | MCF7, MB-MDA-453, MB-MDA-435s |
| 317463 | AA927290 | Hs.130462 | ESTs | 2.04 | NCI-H23, Caco2, NCI-H69 |
| 303460 | AA700155 | Hs.117900 | ESTs | 2.04 | DU145, EB, CALU6 |
| 337435 | | | CH22_FGENES.766-2 | 2.03 | NCI-H345, OVCA-R, LnCap |
| 305464 | AA742425 | | EST singleton (not in UniGene) with exon | 2.03 | CALU6, NCI-H520, NCI-358 |
| 307918 | AI383496 | | EST singleton (not in UniGene) with exon | 2.03 | NCI-H23, BT474, MB-MDA-231 |
| 322209 | H89360 | | EST cluster (not in UniGene) | 2.03 | DU145, OVCA-R, MB-MDA-453 |
| 310295 | AW205198 | Hs.149146 | ESTs | 2.03 | NCI-H23, NCI-H460, NCI-358 |
| 325886 | | | CH.16_hs gi\|5867087 | 2.03 | NCI-H345, NCI-H345, RPWE-2 |
| 329719 | | | CH.14_.p2 gi\|6065785 | 2.03 | NCI-H69, RPWE-2, PRSC_con |
| 309247 | AI972768 | | EST singleton (not in UniGene) with exon | 2.03 | LnCap, PRSC_con, RPWE-2 |
| 328277 | | | CH.07_hs gi\|6004471 | 2.03 | LnCap, RPWE-2, A549 |
| 307296 | AI205705 | Hs.147222 | EST | 2.03 | NCI-H460, NCI-358, NCI-H23 |
| 327203 | | | CH.01_hs gi\|5867447 | 2.03 | HT29, BT474, MB-MDA-231 |
| 306866 | AI086683 | | EST singleton (not in UniGene) with exon | 2.03 | BT474, NCI-H345, HT29 |
| 333339 | | | CH22_FGENES.139_8 | 2.03 | HT29, DU145, CALU6 |
| 323115 | AI921875 | | EST cluster (not in UniGene) | 2.03 | BT474, BT474, MB-MDA-231 |
| 304811 | AA584361 | | EST singleton (not in UniGene) with exon | 2.03 | NCI-H23, NCI-358, NCI-H460 |
| 323372 | AL135125 | Hs.13913 | ESTs | 2.02 | DU145, EB, A549 |
| 312854 | AA828713 | | EST cluster (not in UniGene) | 2.02 | NCI-H345, PRSC_con, PRSC_log |
| 307904 | AI381019 | | EST singleton (not in UniGene) with exon | 2.02 | HT29, MCF7, MB-MDA-453 |
| 332099 | AA608983 | | afsd4.s1 Soares_testis_NHT H sapiens cDN | 2.02 | PRSC_con, NCI-H345, RPWE-2 |
| 324634 | AI684571 | Hs.175831 | ESTs | 2.02 | NCI-H460, Caco2, NCI-358 |
| 335721 | | | CH22_FGENES.599_24 | 2.02 | NCI-H69, PRSC_log, NCI-H345 |
| 312452 | AI692643 | Hs.172749 | ESTs | 2.02 | HT29, Caco2, MB-MDA-231 |
| 325396 | | | CH.12_hs gi\|5866921 | 2.01 | HT29, NCI-H520, NCI-H450 |
| 328770 | | | CH.07_hs gi\|6017031 | 2.01 | NCI-H23, NCI-H460, NCI-358 |
| 335585 | | | CH22_FGENES.581_24 | 2.01 | MB-MDA-453, DU145, MCF7 |
| 335634 | | | CH22_FGENES.584_14 | 2.01 | NCI-H23, NCI-H460, NCI-H69 |
| 338271 | | | CH22_EM:AC005500.GENSCAN.287-1 | 2.01 | MCF7, DU145, PC3 |
| 328607 | | | CH.07_hs gi\|5868233 | 2.01 | NCI-H460, NCI-H23, NCI-358 |
| 307050 | AI147341 | Hs.146734 | EST | 2.01 | NCI-H520, NCI-H23, NCI-358 |
| 334946 | | | CH22_FGENES.465_13 | 2.01 | CALU6, BT474, DU145 |
| 319793 | R56360 | | EST cluster (not in UniGene) | 2.01 | NCI-H460, HT29, NCI-358 |
| 307223 | AI193698 | Hs.184776 | ribosomal protein L23a | 2.01 | NCI-358, NCI-H520, NCI-H23 |
| 312627 | AA344698 | Hs.133169 | ESTs | 2.01 | PC3, LnCap, MB-MDA-231 |
| 329221 | | | CH.X_hs gi\|5868727 | 2.01 | NCI-H345, NCI-H69, NCI-358 |
| 305145 | AA653589 | | EST singleton (not in UniGene) with exon | 2.01 | LnCap, EB, OVCA-R |
| 328428 | | | CH.07_hs gi\|5868417 | 2.01 | NCI-H69, MB-MDA-453, BT474 |
| 305990 | AA888866 | Hs.125919 | EST | 2.01 | NCI-H520, NCI-358, NCI-H23 |
| 319368 | R00003 | Hs.133171 | ESTs | 2 | OVCA-R, LnCap, PC3 |
| 324805 | AA927002 | Hs.131350 | ESTs | 2 | NCI-H460, NCI-H23, NCI-358 |
| 301138 | AA719179 | Hs.189419 | ESTs | 2 | NCI-H69, NCI-H23, PRSC_con |
| 304675 | AA541740 | | EST singleton (not in UniGene) with exon | 2 | NCI-H460, NCI-H520, MB-MDA-231 |
| 326194 | | | CH.17_hs gi\|5867213 | 2 | HT29, NCI-358, 87474 |

TABLE 5
H chip - B survivor vs Met query - up in Mets

| Pkey | Ex Accn | UniG_ID | Complete_Title | Ratio Met/B surv. |
|---|---|---|---|---|
| 102193 | U20758 | Hs. 313 | secreted phosphoprotein 1 (osteopontin; | 5.56 |
| 128530 | AA504343 | Hs. 183475 | Homo sapiens clone 25061 mRNA sequence | 4.62 |
| 129093 | AA262710 | Hs. 108614 | KIAA0627 protein | 4.23 |
| 124690 | R05818 | Hs. 173830 | ESTs | 3.96 |
| 115558 | AA393806 | Hs. 1010 | regulator of mitotic spindle assembly 1 | 3.39 |
| 134261 | AA227678 | Hs. 8084 | Humn DNA sequence from clone 465N24 on c | 3.22 |
| 104792 | AA029288 | Hs. 29147 | ESTs; Highly similar to ZINC FINGER PROT | 3.17 |
| 133770 | M69197 | Hs. 242279 | haptoglobin-related protein | 3.07 |

Pkey: Unique Eos probeset identifier number
ExAccn: Exemplar Accession number, Genbank accession number
UnigeneID: Unigene number
Unigene Title: Unigene gene title

TABLE 6
H chip - B survivor vs Met query - down in Mets

| Pkey | Ex Accn | UniG_ID | Complete_Title | Ratio Met/B surv. |
|---|---|---|---|---|
| 100116 | D00654 | Hs. 77443 | actin; gamma 2; smooth muscle; enteric | 0.07 |
| 101923 | S75256 | | HNL = neutrophil lipocalin [human, ovarian | 0.2 |
| 129982 | M87789 | Hs. 140 | immunoglobulin gamma 3 (Gm marker) | 0.2 |
| 130064 | T67053 | Hs. 181125 | immunoglobulin lambda gene cluster | 0.2 |

Pkey: Unique Eos probeset identifier number
ExAccn: Exemplar Accession number, Genbank accession number
UnigeneID: Unigene number
Unigene Title: Unigene gene title

TABLE 7
I chip - B survivor vs Met query - up in Mets

| Pkey | Ex_Accn | UniG_ID | Title | Ratio Met/B surv |
|---|---|---|---|---|
| 319379 | T91443 | Hs. 193963 | ESTs | 19.65 |
| 321920 | N63915 | | | 11.9 |
| 324302 | AA543008 | Hs. 136806 | ESTs; Weakly similar to !!!! ALU SUBFAMI | 9.31 |
| 314522 | AI732331 | Hs. 187750 | ESTs; Moderately similar to !!!! ALU CLA | 5.79 |
| 331433 | H68097 | Hs. 161023 | EST | 4.79 |
| 324643 | AI436356 | Hs. 130729 | ESTs | 4.59 |
| 332471 | AA416967 | Hs. 120980 | nuclear receptor co-repressor 2 | 4.58 |
| 314915 | AA573072 | Hs. 187748 | ESTs; Weakly similar to !!!! ALU SUBFAMI | 4.3 |
| 321354 | AA078493 | | EST cluster (not in UniGene) | 4.26 |
| 322309 | AF086372 | | EST cluster (not in UniGene) | 3.89 |
| 325100 | T10265 | Hs. 116122 | ESTs; Weakly similar to coded for by C. | 3.81 |
| 314071 | AA192455 | Hs. 188690 | ESTs | 3.74 |
| 315178 | AW362945 | Hs. 162459 | ESTs | 3.66 |
| 330987 | H40988 | Hs. 131965 | ESTs; Weakly similar to !!!! ALU SUBFAMI | 3.51 |
| 337898 | | | CH22_EM: AC005500.GENSCAN.56-5 | 3.21 |
| 319403 | T98413 | | EST cluster (not in UniGene) | 3.2 |
| 331469 | N22273 | Hs. 39140 | ESTs | 3.15 |
| 331549 | N56866 | Hs. 237507 | EST | 3.14 |
| 331644 | T99544 | Hs. 173734 | ESTs; Weakly similar to !!!! ALU CLASS B | 3.14 |
| 313220 | AI971981 | Hs. 118241 | ESTs | 3.04 |

Pkey: Unique Eos probeset identifier number
ExAccn: Exemplar Accession number, Genbank accession number
UnigeneID: Unigene number
Unigene Title: Unigene gene title

TABLE 8
I chip - B survivor vs Met query - down in Mets

| Pkey | Ex_Accn | UniG_ID | Title | Ratio Met/B surv |
|---|---|---|---|---|
| 333658 | | | CH22_FGENES.241_4 | 0.06 |
| 333657 | | | CH22_FGENES.241_2 | 0.07 |
| 333654 | | | CH22_FGENES.240_2 | 0.07 |
| 332859 | | | CH22_FGENES.27_2 | 0.07 |
| 333656 | | | CH22_FGENES.240_4 | 0.07 |
| 304480 | AA430373 | | EST singleton (not in UniGene) with exon | 0.08 |
| 333737 | | | CH22_FGENES.261_1 | 0.09 |
| 308601 | AI719930 | | EST singleton (not in UniGene) with exon | 0.1 |
| 334030 | | | CH22_FGENES.320_2 | 0.1 |
| 333637 | | | CH22_FGENES.229_2 | 0.13 |
| 302347 | AF039400 | Hs. 194659 | chloride channel; calcium activated; fam | 0.16 |
| 333653 | | | CH22_FGENES.239_2 | 0.16 |
| 333635 | | | CH22_FGENES.228_2 | 0.19 |
| 333647 | | | CH22_FGENES.235_2 | 0.19 |
| 307588 | AI285535 | | EST singleton (not in UniGene) with exon | 0.2 |
| 337954 | | | CH22_EM: AC005500.GENSCAN.96-3 | 0.2 |
| 333588 | | | CH22_FGENES.206_2 | 0.21 |
| 320244 | AA296922 | Hs. 129778 | gastrointestinal peptide | 0.22 |
| 333642 | | | CH22_FGENES.231_2 | 0.23 |
| 337951 | | | CH22_EM: AC005500.GENSCAN.94-1 | 0.23 |
| 333730 | | | CH22_FGENES.258_1 | 0.23 |
| 333646 | | | CH22_FGENES.234_2 | 0.24 |

Pkey: Unique Eos probeset identifier number
ExAccn: Exemplar Accession number, Genbank accession number
UnigeneID: Unigene number
Unigene Title: Unigene gene title

TABLE 9
H chip - B survivor vs Met query - up in Mets

| | | | | Median Mets AI vs |
|---|---|---|---|---|
| Pkey | Ex Accn | UniGID | Complete_Title | Median B-Sur AI |
| 100655 | HG2841-HT2970 | | Albumin, Alt. Splice 5 | 11.98 |
| 124875 | R70506 | Hs. 207693 | ESTs; Weakly similar to !!!! ALU SUBFAMI | 9.21 |
| 102193 | U20758 | Hs. 313 | secreted phosphoprotein 1 (osteopontin; | 6.73 |
| 100654 | HG2841-HT2969 | | Albumin, Alt. Splice 3, Missplicing In Alloalbumin Venezia | 6.18 |
| 118828 | N79496 | Hs. 50824 | EST | 5.93 |
| 128046 | AA873285 | Hs. 137947 | ESTs | 5.9 |
| 128896 | D14446 | Hs. 107 | fibrinogen-like 1 | 5.17 |
| 127917 | AA211895 | Hs. 118831 | EST; Highly similar to dJ1163J1.2.1 [H.s | 5.11 |
| 125090 | T91518 | | ye20f05.s1 Stratagene lung (#937210) Hom | 4.47 |
| 118579 | N68905 | | small inducible cytokine A5 (RANTES) | 4.23 |
| 123526 | AA608657 | | ESTs; Moderately similar to !!!! ALU SUB | 4.21 |
| 128062 | AA379500 | Hs. 193155 | ESTs | 4.14 |
| 119174 | R71234 | | yi54c08.s1 Soares placenta Nb2HP Homo sa | 4.11 |
| 128530 | AA504343 | Hs. 183475 | Homo sapiens clone 25061 mRNA sequence | 4.09 |
| 119404 | T92950 | | ye27c10.s1 Stratagene lung (#937210) Hom | 3.98 |
| 118475 | N66845 | Hs. 165411 | ESTs; Weakly similar to !!!! ALU CLASS B | 3.96 |
| 129974 | K00629 | Hs. 199300 | Human kpni repeat mma (cdna clone pcd-k | 3.87 |
| 108888 | AA135606 | Hs. 189384 | ESTs; Weakly similar to !!!! ALU SUBFAMI | 3.85 |
| 123963 | C13961 | Hs. 210115 | EST | 3.8 |
| 123523 | AA608588 | Hs. 193634 | ESTs | 3.76 |
| 128230 | AA984074 | Hs. 176757 | ESTs | 3.75 |
| 124090 | H09570 | Hs. 143032 | ESTs; Weakly similar to neuronal thread | 3.67 |
| 124690 | R05818 | Hs. 173830 | ESTs | 3.58 |
| 134261 | AA227678 | Hs. 8084 | Human DNA sequence from clone 465N24 on | 3.57 |
| 126917 | AA176225 | Hs. 193929 | ESTs | 3.52 |
| 126050 | H27267 | Hs. 75860 | hydroxyacyl-Coenzyme A dehydrogenase/3-k | 3.45 |
| 126649 | AA856990 | Hs. 125058 | ESTs | 3.42 |
| 115096 | AA255991 | Hs. 175319 | ESTs | 3.4 |
| 129906 | H39216 | Hs. 239970 | ESTs; Weakly similar to ZNF91L [H.sapien | 3.38 |
| 123022 | AA480909 | | aa28f10.s1 NCI_CGAP_GCB1 Homo sapiens cD | 3.38 |
| 106145 | AA424791 | Hs. 5734 | KIAA0679 protein | 3.38 |
| 125191 | W67257 | Hs. 138871 | ESTs; Weakly similar to !!!! ALU CLASS B | 3.36 |
| 108836 | AA132061 | Hs. 222727 | ESTs; Weakly similar to ubiquitous TPR m | 3.3 |
| 128710 | J04813 | Hs. 104117 | cytochrome P450; subfamily IIIA (niphedi | 3.27 |
| 123460 | AA598981 | Hs. 251122 | EST | 3.25 |
| 133735 | AC002045 | Hs. 251928 | nuclear pore complex interacting protein | 3.24 |
| 124696 | R06273 | Hs. 186467 | ESTs; Moderately similar to !!!! ALU SUB | 3.24 |
| 120748 | AA303153 | Hs. 237994 | EST; Weakly similar to !!!! ALU SUBFAMIL | 3.21 |
| 133770 | M69197 | Hs. 242279 | haptoglobin-related protein | 3.17 |
| 128336 | AI242720 | Hs. 146043 | ESTs; Weakly similar to alternatively sp | 3.14 |
| 135357 | AA235803 | Hs. 79572 | cathepsin D (lysosomal aspartyl protease | 3.12 |
| 128088 | R02443 | Hs. 186467 | ESTs; Moderately similar to !!!! ALU SUB | 3.08 |
| 124055 | F10904 | Hs. 100516 | Homo sapiens clone 23605 mRNA sequence | 3.06 |
| 124896 | R82063 | Hs. 101594 | EST | 3.06 |
| 127598 | AA610677 | Hs. 168851 | ESTs | 3.04 |
| 116802 | H44061 | Hs. 194026 | ESTs | 3.01 |

Pkey: Unique Eos probeset identifier number
ExAccn: Exemplar Accession number, Genbank accession number
UnigeneID: Unigene number
Unigene Title: Unigene gene title

TABLE 10
H chip - B survivor vs Met query - Down in Mets

| Pkey | Ex Accn | UniG_ID | Complete_Title | Ratio Met/B surv. |
|---|---|---|---|---|
| 100116 | D00654 | Hs. 77443 | actin; gamma 2; smooth muscle; enteric | 0.09 |
| 130064 | T67053 | Hs. 181125 | immunoglobulin lambda gene cluster | 0.11 |
| 129982 | M87789 | Hs. 140 | immunoglobulin gamma 3 (Gm marker) | 0.12 |
| 131219 | C00476 | Hs. 24395 | small inducible cytokine subfamily B (Cy | 0.13 |
| 133806 | M12759 | Hs. 76325 | Human Ig J chain gene | 0.17 |
| 132982 | L02326 | Hs. 198118 | immunoglobulin lambda-like polypeptide 2 | 0.18 |
| 131713 | X57809 | Hs. 181125 | immunoglobulin lambda gene cluster | 0.18 |
| 131791 | S71043 | Hs. 32225 | immunoglobulin alpha 1 | 0.2 |
| 133725 | V00563 | Hs. 179543 | immunoglobulin mu | 0.22 |
| 101923 | S75256 | | HNL = neutrophil lipocalin [human, ovarian | 0.23 |
| 101461 | M22430 | Hs. 76422 | phospholipase A2; group IIA (platelets; | 0.24 |
| 103448 | X99133 | Hs. 204238 | lipocalin 2 (oncogene 24p3) | 0.24 |

Pkey: Unique Eos probeset identifier number
ExAccn: Exemplar Accession number, Genbank accession number
UnigeneID: Unigene number
Unigene Title: Unigene gene title

TABLE 11
H chip - Met vs Normal query - up in Mets

| | | | | Median Mets Al vs |
|---|---|---|---|---|
| Pkey | Ex Accn | UniG_ID | Complete_Title | Median Normal Al |
| 100655 | HG2841-HT2970 | | Albumin, Alt. Splice 5 | 15.91 |
| 102193 | U20758 | Hs. 313 | secreted phosphoprotein 1 (osteopontin; | 6.83 |
| 124875 | R70506 | Hs. 207693 | ESTs; Weakly similar to !!!! ALU SUBFAMI | 6.68 |
| 100654 | HG2841-HT2969 | | Albumin, Alt. Splice 3, Missplicing In Alloalbumin Venezia | 5.28 |
| 124059 | F13673 | Hs. 99769 | ESTs | 5.11 |
| 128896 | D14446 | Hs. 107 | fibrinogen-like 1 | 5.05 |
| 134453 | X70683 | Hs. 83484 | SRY (sex determining region Y)-box 4 | 4.82 |
| 131564 | AA491465 | Hs. 28792 | ESTs | 4.78 |
| 127917 | AA211895 | Hs. 118831 | EST; Highly similar to dJ1163J1.2.1 [H.s | 4.76 |
| 115096 | AA255991 | Hs. 175319 | ESTs | 4.67 |
| 104558 | R56678 | Hs. 88959 | Human DNA sequence from clone 967N21 on | 4.63 |
| 123526 | AA608657 | | ESTs; Moderately similar to !!!! ALU SUB | 4.61 |
| 125090 | T91518 | | ye20f05.s1 Stratagene lung (#937210) Hom | 4.59 |
| 129666 | M77349 | Hs. 118787 | transforming growth factor, beta-induced | 4.58 |
| 118828 | N79496 | Hs. 50824 | EST | 4.56 |
| 128046 | AA873285 | Hs. 137947 | ESTs | 4.45 |
| 133421 | AA436560 | Hs. 7327 | claudin 1 | 4.09 |
| 129158 | J05257 | Hs. 109 | dipeptidase 1 (renal) | 4.04 |
| 128062 | AA379500 | Hs. 193155 | ESTs | 4.03 |
| 124696 | R06273 | Hs. 186467 | ESTs; Moderately similar to !!!! ALU SUB | 4.01 |
| 118475 | N66845 | Hs. 165411 | ESTs; Weakly similar to !!!! ALU CLASS B | 3.96 |
| 104755 | AA024482 | Hs. 9029 | DKFZP434G032 protein | 3.83 |
| 104978 | AA088458 | Hs. 19322 | ESTs | 3.74 |
| 118579 | N68905 | | small inducible cytokine A5 (RANTES) | 3.7 |
| 123796 | AA620390 | Hs. 247444 | ESTs | 3.62 |
| 127240 | AA888387 | Hs. 243845 | ESTs; Moderately similar to !!!! ALU SUB | 3.61 |
| 104105 | AA422123 | Hs. 42457 | ESTs | 3.55 |
| 129349 | D86974 | Hs. 110613 | KIAA0220 protein | 3.54 |
| 119329 | T51832 | | ESTs; Moderately similar to !!!! ALU SUB | 3.53 |
| 114617 | AA084148 | Hs. 110659 | ESTs | 3.52 |
| 123143 | AA487595 | | aa95e2.s1 Stratagene fetal retina 93722 | 3.48 |
| 103119 | X63629 | Hs. 2877 | cadherin 3; P-cadherin (placental) | 3.48 |
| 119404 | T92950 | | ye27c10.s1 Stratagene lung (#937210) Hom | 3.47 |
| 123963 | C13961 | Hs. 210115 | EST | 3.47 |
| 116480 | C14088 | Hs. 195188 | glyceraldehyde-3-phosphate dehydrogenase | 3.4 |
| 108836 | AA132061 | Hs. 222727 | ESTs; Weakly similar to ubiquitous TPR m | 3.39 |
| 120748 | AA303153 | Hs. 237994 | EST; Weakly similar to !!!! ALU SUBFAMIL | 3.38 |
| 133770 | M69197 | Hs. 242279 | haptoglobin-related protein | 3.38 |
| 132358 | X60486 | Hs. 46423 | H4 histone family; member G | 3.37 |
| 127759 | AI369384 | | arylsulfatase D | 3.37 |
| 129095 | L12350 | Hs. 108623 | thrombospondin 2 | 3.37 |
| 128261 | AI061213 | Hs. 13179 | ESTs; Moderately similar to !!!! ALU SUB | 3.36 |
| 126908 | AA169866 | | ESTs; Weakly similar to !!!! ALU SUBFAMI | 3.36 |
| 128954 | N32118 | Hs. 209100 | DKFZP434C171 protein | 3.34 |
| 119174 | R71234 | | yi54c08.s1 Soares placenta Nb2HP Homo sa | 3.33 |
| 106687 | AA463234 | Hs. 119387 | KIAA0792 gene product | 3.32 |
| 128230 | AA984074 | Hs. 176757 | ESTs | 3.3 |
| 126649 | AA856990 | Hs. 125058 | ESTs | 3.25 |
| 124620 | N74051 | Hs. 194092 | ESTs; Weakly similar to !!!! ALU SUBFAMI | 3.24 |
| 135427 | | | AFFX control: human alu repeats | 3.23 |
| 129967 | H99653 | Hs. 138618 | ESTs | 3.22 |
| 125191 | W67257 | Hs. 138871 | ESTs; Weakly similar to !!!! ALU CLASS B | 3.2 |
| 124684 | R02401 | Hs. 221078 | ESTs | 3.2 |
| 128010 | AA856953 | Hs. 23348 | S-phase kinase-associated protein 2 (p45 | 3.17 |
| 119423 | T99544 | Hs. 173734 | ESTs; Weakly similar to !!!! ALU CLASS B | 3.16 |
| 123022 | AA480909 | | aa28f10.s1 NCI_CGAP_GCB1 Homo sapiens cD | 3.15 |
| 103654 | Z70759 | | H. sapiens mitochondrial 16S rRNA gene (p | 3.13 |
| 128336 | AI242720 | Hs. 146043 | ESTs; Weakly similar to alternatively sp | 3.12 |
| 124690 | R05818 | Hs. 173830 | ESTs | 3.1 |
| 129791 | F02778 | Hs. 173887 | KIAA0876 protein | 3.07 |
| 114472 | AA028924 | Hs. 177407 | ESTs; Weakly similar to !!!! ALU SUBFAMI | 3.07 |
| 115429 | AA284139 | Hs. 89295 | EST | 3.06 |
| 130020 | AA433930 | Hs. 240443 | ESTs; Weakly similar to !!!! HNK-1 sulfotrans | 3.06 |
| 126050 | H27267 | Hs. 75860 | hydroxyacyl-Coenzyme A dehydrogenase/3-k | 3.05 |
| 129906 | H39216 | Hs. 239970 | ESTs; Weakly similar to ZNF91L [H.sapien | 3.04 |
| 123422 | AA598484 | Hs. 238476 | EST | 3.03 |
| 103059 | X57351 | Hs. 174195 | interferon induced transmembrane protein | 3.02 |
| 124253 | H69742 | Hs. 102201 | ESTs | 3.02 |
| 123523 | AA608588 | Hs. 193634 | ESTs | 3.02 |
| 132669 | AA188378 | Hs. 54602 | ESTs; Weakly similar to 60S RIBOSOMAL PR | 3.02 |
| 123196 | AA489250 | Hs. 59403 | serine palmitoyltransferase; subunit II | 3.01 |
| 122948 | AA477483 | | zu44h2.s1 Soares ovary tumor NbHOT Homo | 3.01 |
| 119053 | R11501 | | yf28f1.s1 Soares fetal liver spleen 1NFL | 3.01 |
| 125953 | H40829 | | yo05d11.r1 Soares adult brain N2b5HB55Y | 3 |
| 119155 | R61715 | Hs. 138237 | ESTs | 3 |

Pkey: Unique Eos probeset identifier number
ExAccn: Exemplar Accession number, Genbank accession number
UnigeneID: Unigene number
Unigene Title: Unigene gene title

TABLE 12
H chip - Met vs Normal query - down in Mets

| Pkey | Ex Accn | UniG_ID | Complete_Title | Median Mets Al vs Median Normal Al |
|---|---|---|---|---|
| 103466 | Y00339 | Hs. 155097 | carbonic anhydrase II | 0.01 |
| 104258 | AF007216 | Hs. 5462 | solute carrier family 4; sodium bicarbon | 0.02 |
| 108999 | AA156064 | Hs. 72115 | ESTs | 0.04 |
| 101046 | K01160 | | Accession not listed in Genbank | 0.04 |
| 133565 | H57056 | Hs. 204831 | ESTs | 0.05 |
| 101346 | L76465 | Hs. 77348 | hydroxyprostaglandin dehydrogenase 15-(N | 0.05 |
| 123137 | AA487468 | Hs. 100686 | ESTs; Weakly similar to secreted cement | 0.05 |
| 134534 | X73501 | Hs. 84905 | H. Sapiens mRNA for cytokeratin 20 | 0.05 |
| 118823 | N79237 | Hs. 50813 | ESTs; Weakly similar to long chain fatty | 0.06 |
| 102095 | U11313 | Hs. 75760 | sterol carrier protein 2 | 0.06 |
| 111855 | R37362 | Hs. 21351 | ESTs | 0.06 |
| 129105 | AA224351 | Hs. 108681 | ESTs | 0.07 |
| 130320 | U19495 | Hs. 237356 | stromal cell-derived factor 1 | 0.07 |
| 113778 | W15263 | Hs. 5422 | ESTs | 0.07 |
| 116786 | H25836 | Hs. 83429 | tumor necrosis factor (ligand) superfami | 0.07 |
| 100116 | D00654 | Hs. 77443 | actin; gamma 2; smooth muscle; enteric | 0.07 |
| 104636 | AA004415 | Hs. 106106 | ESTs | 0.07 |
| 107032 | AA599472 | Hs. 247309 | succinate-CoA ligase; GDP-forming; beta | 0.08 |
| 106605 | AA457718 | Hs. 21103 | Homo sapiens mRNA; cDNA DKFZp564B076 (fr | 0.08 |
| 128906 | AA487557 | Hs. 10706 | ESTs | 0.08 |
| 130016 | AA055811 | Hs. 143131 | transmembrane glycoprotein | 0.08 |
| 113523 | T90037 | Hs. 16686 | ESTs | 0.08 |
| 102638 | U67319 | Hs. 9216 | caspase 7; apoptosis-related cysteine pr | 0.09 |
| 124308 | H93575 | Hs. 227146 | Homo sapiens mRNA; cDNA DKFZp564J142 (fr | 0.09 |
| 129519 | AA298786 | Hs. 112242 | ESTs | 0.09 |
| 134749 | L10955 | Hs. 89485 | carbonic anhydrase IV | 0.09 |
| 130366 | L11708 | Hs. 155109 | hydroxysteroid (17-beta) dehydrogenase 2 | 0.09 |
| 109272 | AA195718 | Hs. 86030 | ESTs | 0.09 |
| 102124 | U14528 | Hs. 29981 | solute carrier family 26 (sulfate transp | 0.1 |
| 132711 | N73702 | Hs. 238927 | ESTs | 0.1 |
| 131861 | D11925 | Hs. 184245 | KIAA0929 protein Msx2 interacting nuclea | 0.1 |
| 133806 | M12759 | Hs. 76325 | Human Ig J chain gene | 0.1 |
| 102571 | U60115 | | Homo sapiens skeletal muscle LIM-protein | 0.1 |
| 114846 | AA234929 | Hs. 44343 | ESTs | 0.11 |
| 131328 | V01512 | Hs. 25647 | v-fos FBJ murine osteosarcoma viral onco | 0.11 |
| 106569 | AA455983 | Hs. 117816 | sorcin | 0.11 |
| 103542 | Z11793 | Hs. 3314 | selenoprotein P; plasma; 1 | 0.11 |
| 128915 | C02386 | Hs. 107139 | ESTs | 0.11 |
| 120914 | AA377254 | Hs. 97107 | EST | 0.11 |
| 130867 | J04093 | Hs. 2056 | UDP glycosyltransferase 1 | 0.11 |
| 110837 | N30796 | Hs. 17424 | ESTs; Weakly similar to semaphorin F [H. | 0.12 |
| 101877 | M97496 | Hs. 778 | guanylate cyclase activator 1B (retina) | 0.12 |
| 132617 | AA171913 | Hs. 5338 | carbonic anhydrase XII | 0.12 |
| 129113 | AA147646 | Hs. 108740 | DKFZP586A0522 protein | 0.12 |
| 133435 | T23983 | Hs. 7365 | ESTs | 0.13 |
| 132836 | F09557 | Hs. 57929 | slit (Drosophila) homolog 3 | 0.13 |
| 125832 | AA628600 | Hs. 117587 | ESTs | 0.13 |
| 104613 | AA001049 | Hs. 24713 | Homo sapiens mRNA; cDNA DKFZp586G0123 (f | 0.13 |
| 132903 | AA235404 | Hs. 5985 | Homo sapiens clone 25186 mRNA sequence | 0.13 |
| 119479 | W32094 | Hs. 55501 | ESTs | 0.14 |
| 131273 | AA421139 | Hs. 173542 | ESTs | 0.14 |
| 106674 | AA461303 | Hs. 7946 | DKFZP586D1519 protein | 0.14 |
| 108980 | AA151676 | Hs. 33455 | peptidyl arginine deiminase; type II | 0.14 |
| 103211 | X73079 | Hs. 205126 | polymeric immunoglobulin receptor | 0.14 |
| 131219 | C00476 | Hs. 24395 | small inducible cytokine subfamily B (Cy | 0.15 |
| 116459 | AA621399 | Hs. 64193 | ESTs | 0.15 |
| 130219 | R77539 | Hs. 15285 | ESTs | 0.15 |
| 113863 | W68388 | Hs. 21288 | ESTs; Weakly similar to KIAA0704 protein | 0.15 |
| 101564 | M32886 | Hs. 117816 | sorcin | 0.15 |
| 109502 | AA233837 | Hs. 44755 | ESTs; Weakly similar to membrane glycopr | 0.15 |
| 107222 | D51235 | Hs. 82689 | tumor rejection antigen (gp96) 1 | 0.15 |
| 135237 | AA454930 | Hs. 9691 | ESTs | 0.15 |
| 112483 | R66534 | Hs. 28403 | ESTs | 0.15 |
| 132387 | R70914 | Hs. 8997 | heat shock 70 kD protein 1 | 0.15 |
| 130343 | AA490262 | Hs. 15485 | ESTs; Weakly similar to APICAL-LIKE PROT | 0.16 |
| 105496 | AA256323 | Hs. 25264 | DKFZP434N126 protein | 0.16 |
| 104037 | AA372630 | Hs. 100347 | differentially expressed in hematopoieti | 0.16 |
| 101461 | M22430 | Hs. 76422 | phospholipase A2; group IIA (platelets; | 0.16 |
| 116551 | D20458 | Hs. 229071 | EST | 0.16 |
| 133889 | AA099391 | Hs. 211582 | myosin; light polypeptide kinase | 0.16 |
| 103653 | Z70295 | Hs. 32966 | guanylate cyclase activator 2B (uroguany | 0.16 |
| 101070 | L02785 | Hs. 1650 | down-regulated in adenoma | 0.17 |
| 131501 | AA121127 | Hs. 181307 | H3 histone; family 3A | 0.17 |
| 133515 | X98311 | Hs. 74466 | carcinoembryonic antigen-related cell ad | 0.17 |
| 108604 | AA099820 | Hs. 49696 | ESTs | 0.17 |
| 132982 | L02326 | Hs. 198118 | immunoglobulin lambda-like polypeptide 2 | 0.17 |
| 131676 | C20785 | Hs. 30514 | ESTs | 0.17 |
| 134675 | AA250745 | Hs. 87773 | protein kinase; cAMP-dependent; catalyti | 0.17 |
| 133441 | M82962 | Hs. 179704 | meprin A; alpha (PABA peptide hydrolase) | 0.18 |
| 130455 | X17059 | Hs. 155956 | N-acetyltransferase 1 (arylamine N-acety | 0.18 |
| 131734 | D62965 | Hs. 31297 | ESTs | 0.18 |
| 100749 | HG3521-HT3715 | | Ras-Related Protein Rap1b | 0.18 |
| 116724 | F13665 | Hs. 65641 | ESTs | 0.18 |
| 129265 | X68277 | Hs. 171695 | dual specificity phosphatase 1 | 0.18 |
| 102347 | U37518 | Hs. 83429 | tumor necrosis factor (ligand) superfami | 0.18 |
| 114542 | AA055768 | Hs. 122576 | ESTs | 0.18 |
| 123900 | AA621223 | Hs. 112953 | EST | 0.19 |
| 121780 | AA422086 | Hs. 124660 | ESTs | 0.19 |
| 115662 | AA405715 | Hs. 64179 | hypothetical protein | 0.19 |
| 113803 | W42789 | Hs. 31446 | ESTs | 0.19 |
| 105493 | AA256268 | Hs. 10283 | ESTs | 0.19 |
| 113195 | T57112 | | yc20g11.s1 Stratagene lung(#937210) Hom | 0.19 |
| 129462 | D84239 | Hs. 111732 | IgG Fc binding protein | 0.19 |
| 133664 | X86693 | Hs. 75445 | hevin | 0.2 |
| 126180 | R18070 | Hs. 3712 | ubiquinol-cytochrome c reductase; Rieske | 0.2 |
| 100687 | HG3115-HT3291 | | Golli-Mbp (Gb: L18862) | 0.2 |
| 130064 | T67053 | Hs. 181125 | immunoglobulin lambda gene cluster | 0.2 |
| 101367 | M12963 | Hs. 73843 | alcohol dehydrogenase 1 (class I); alpha | 0.2 |
| 132254 | L20826 | Hs. 430 | plastin 1 (I isoform) | 0.2 |
| 105646 | AA282147 | Hs. 5888 | ESTs | 0.2 |
| 132883 | AA047151 | Hs. 5897 | Homo sapiens mRNA; cDNA DKFZp586P1622 (f | 0.21 |
| 132618 | AA253330 | Hs. 5344 | adaptor-related protein complex 1; gamma | 0.21 |
| 108931 | AA147186 | Hs. 250746 | ESTs | 0.22 |
| 131421 | X64177 | Hs. 2667 | metallothionein 1H | 0.22 |
| 107295 | T34527 | Hs. 80120 | UDP-N-acetyl-alpha-D-galactosamine:polyp | 0.22 |
| 103576 | Z26317 | Hs. 2631 | desmoglein 2 | 0.22 |
| 105173 | AA182030 | Hs. 8364 | ESTs | 0.22 |
| 134843 | H60595 | Hs. 90061 | progesterone binding protein | 0.22 |
| 102009 | U02680 | Hs. 82643 | protein tyrosine kinase 9 | 0.23 |
| 123997 | D51171 | Hs. 78902 | voltage-dependent anion channel 2 | 0.23 |
| 106609 | AA458652 | Hs. 32181 | ESTs | 0.23 |
| 101300 | L40391 | Hs. 6445 | Homo sapiens (clone s153) mRNA fragment | 0.23 |
| 129717 | AA481670 | Hs. 12150 | ESTs; Weakly similar to retinal short-ch | 0.23 |
| 108565 | AA085342 | Hs. 1526 | ATPase; Ca++ transporting; cardiac muscl | 0.23 |
| 121314 | AA402799 | Hs. 182538 | ESTs | 0.23 |
| 124803 | R45480 | Hs. 164866 | cyclin K | 0.23 |
| 130208 | AA620556 | Hs. 15250 | peroxisomal D3;D2-enoyl-CoA isomerase | 0.23 |
| 132888 | AA490775 | Hs. 5920 | UDP-N-acetylglucosamine-2-epimerase/N-ac | 0.23 |
| 132720 | Z69881 | Hs. 5541 | ATPase; Ca++ transporting; ubiquitous | 0.23 |
| 102239 | U26726 | Hs. 1376 | hydroxysteroid (11-beta) dehydrogenase 2 | 0.23 |
| 115764 | AA421562 | Hs. 91011 | anterior gradient 2 (Xenepus laevis) hom | 0.24 |
| 130558 | H96654 | Hs. 15984 | ESTs; Weakly similar to gene pp21 protei | 0.24 |
| 122666 | AA455052 | Hs. 99387 | ESTs | 0.24 |
| 134495 | D63477 | Hs. 84087 | KIAA0143 protein | 0.24 |
| 124017 | F02202 | Hs. 100960 | ESTs | 0.24 |
| 106925 | AA491261 | Hs. 37558 | Homo sapiens clone 23923 mRNA sequence | 0.24 |
| 115187 | AA261805 | Hs. 44021 | ESTs | 0.24 |
| 105309 | AA233790 | Hs. 4104 | ESTs; Weakly similar to cDNA EST yk386g7 | 0.24 |
| 124457 | N50114 | Hs. 128704 | ESTs | 0.24 |
| 130616 | AA233763 | Hs. 16726 | Homo sapiens mRNA; cDNA DKFZp564A132 (fr | 0.25 |
| 105795 | AA369245 | Hs. 17448 | ESTs; Weakly similar to !!!! ALU SUBFAMI | 0.25 |
| 134579 | N23222 | Hs. 85963 | CD36 antigen (collagen type I receptor; | 0.25 |

Pkey: Unique Eos probeset identifier number
ExAccn: Exemplar Accession number, Genbank accession number
UnigeneID: Unigene number
Unigene Title: Unigene gene title

TABLE 13
H chip - Met vs Normal query - up in Mets

| Pkey | Ex Accn | UniG_ID | Complete_Title | Ratio Met/Normal |
|---|---|---|---|---|
| 102193 | U20758 | Hs. 313 | secreted phosphoprotein 1 (osteopontin; | 8.457 |
| 111307 | N73988 | Hs. 37477 | ESTs; Weakly similar to CGI-141 protein | 6.05 |
| 103119 | X63629 | Hs. 2877 | cadherin 3; P-cadherin (placental) | 5.207 |
| 131564 | AA491456 | Hs. 28792 | ESTs | 5.136 |
| 119729 | W69747 | Hs. 94806 | KIAA1062 protein | 4.667 |
| 124059 | F13673 | Hs. 99769 | ESTs | 4.398 |
| 123987 | C21171 | Hs. 95497 | ESTs; Weakly similar to GLUCOSE TRANSPOR | 4.292 |
| 128817 | N47524 | Hs. 28491 | spermidine/spermine N1-acetyltransferase | 3.964 |
| 133770 | M69197 | Hs. 242279 | haptoglobin-related protein | 3.823 |
| 130412 | AA406554 | Hs. 241572 | golgi autoantigen; golgin subfamily a; 5 | 3.719 |
| 104755 | AA024482 | Hs. 9029 | DKFZP434G032 protein | 3.702 |
| 132676 | AA283035 | Hs. 54813 | ESTs | 3.645 |
| 134453 | X70683 | Hs. 83484 | SRY (sex determining region Y)-box 4 | 3.581 |
| 124690 | R05818 | Hs. 173830 | ESTs | 3.446 |
| 106949 | AA496805 | Hs. 177425 | KIAA0964 protein | 3.42 |
| 130724 | AA370091 | Hs. 179680 | ESTs | 3.402 |
| 128992 | R49693 | Hs. 107708 | ESTs | 3.32 |
| 133421 | AA436560 | Hs. 7327 | claudin 1 | 3.255 |
| 103047 | X55990 | Hs. 73839 | ribonuclease; RNase A family; 3 (eosinop | 3.229 |
| 102990 | X51441 | Hs. 181062 | serum amyloid A1 | 3.149 |
| 115429 | AA284139 | Hs. 89295 | EST | 3.114 |
| 129158 | J05257 | Hs. 109 | dipeptidase 1 (renal) | 3.019 |
| 123533 | AA608751 | Hs. 244904 | ESTs; Weakly similar to !!!! ALU SUBFAMI | 3.011 |

Pkey: Unique Eos probeset identifier number
ExAccn: Exemplar Accession number, Genbank accession number
UnigeneID: Unigene number
Unigene Title: Unigene gene title

TABLE 14
H chip - Met vs Normal query - down in Mets

| PkeyY | Ex Accn | UniG_ID | Complete_Title | Ratio Met/Normal |
|---|---|---|---|---|
| 103466 | Y00339 | Hs. 155097 | carbonic anhydrase II | 0.012 |
| 104258 | AF007216 | Hs. 5462 | solute carrier family 4; sodium bicarbon | 0.025 |
| 108999 | AA156064 | Hs. 72115 | ESTs | 0.034 |
| 101046 | K01160 | | Accession not listed in Genbank | 0.041 |
| 133565 | H57056 | Hs. 204831 | ESTs | 0.042 |
| 101346 | L76465 | Hs. 77348 | hydroxyprostaglandin dehydrogenase 15-(N | 0.043 |
| 102095 | U11313 | Hs. 75760 | sterol carrier protein 2 | 0.054 |
| 111855 | R37362 | Hs. 21351 | ESTs | 0.055 |
| 130320 | U19495 | Hs. 237356 | stromal cell-derived factor 1 | 0.058 |
| 123137 | AA487468 | Hs. 100686 | ESTs; Weakly similar to secreted cement | 0.06 |
| 107222 | D51235 | Hs. 82689 | tumor rejection antigen (gp96) 1 | 0.06 |
| 102638 | U67319 | Hs. 9216 | caspase 7; apoptosis-related cysteine pr | 0.063 |
| 128906 | AA487557 | Hs. 10706 | ESTs | 0.065 |
| 129105 | AA224351 | Hs. 108681 | ESTs | 0.069 |
| 110837 | N30796 | Hs. 17424 | ESTs; Weakly similar to semaphorin F [H. | 0.069 |
| 100116 | D00654 | Hs. 77443 | actin; gamma 2; smooth muscle; enteric | 0.071 |
| 116786 | H25836 | Hs. 83429 | tumor necrosis factor (ligand) superfami | 0.074 |
| 130867 | J04093 | Hs. 2056 | UDP glycosyltransferase 1 | 0.075 |
| 132836 | F09557 | Hs. 57929 | slit (Drosophila) homolog 3 | 0.076 |
| 131861 | D11925 | Hs. 184245 | KIAA0929 protein Msx2 interacting nuclea | 0.081 |
| 106674 | AA461303 | Hs. 7946 | DKFZP586D1519 protein | 0.084 |
| 109272 | AA195718 | Hs. 86030 | ESTs | 0.088 |
| 132711 | N73702 | Hs. 238927 | ESTs | 0.091 |
| 106569 | AA455983 | Hs. 117816 | sorcin | 0.092 |
| 104636 | AA004415 | Hs. 106106 | ESTs | 0.093 |
| 118823 | N79237 | Hs. 50813 | ESTs; Weakly similar to long chain fatty | 0.094 |
| 134534 | X73501 | Hs. 84905 | H. Sapiens mRNA for cytokeratin 20 | 0.095 |
| 119479 | W32094 | Hs. 55501 | ESTs | 0.096 |
| 113778 | W15263 | Hs. 5422 | ESTs | 0.098 |
| 128482 | U83908 | Hs. 100407 | programmed cell death 4 | 0.102 |
| 124653 | N92884 | Hs. 109641 | ESTs | 0.106 |
| 133407 | AA093348 | Hs. 7306 | secreted frizzled-related protein 1 | 0.108 |
| 135237 | AA454930 | Hs. 9691 | ESTs | 0.109 |
| 116250 | AA480975 | Hs. 44829 | ESTs | 0.111 |
| 132617 | AA171913 | Hs. 5338 | carbonic anhydrase XII | 0.112 |
| 131273 | AA421139 | Hs. 173542 | ESTs | 0.113 |
| 116710 | F10577 | Hs. 70312 | ESTs | 0.114 |
| 131791 | S71043 | Hs. 32225 | immunoglobulin alpha 1 | 0.114 |
| 112483 | R66534 | Hs. 28403 | ESTs | 0.115 |
| 132017 | W67251 | Hs. 37331 | Homo sapiens vav 3 oncogene (VAV3) mRNA | 0.116 |
| 124308 | H93575 | Hs. 227146 | Homo sapiens mRNA; cDNA DKFZp564J142 (fr | 0.117 |
| 114846 | AA234929 | Hs. 44343 | ESTs | 0.119 |
| 116551 | D20458 | Hs. 229071 | EST | 0.12 |
| 105299 | AA233511 | Hs. 194720 | ATP-binding cassette; sub-family G (WHIT | 0.122 |
| 130366 | L11708 | Hs. 155109 | hydroxysteroid (17-beta) dehydrogenase 2 | 0.122 |
| 133806 | M12759 | Hs. 76325 | Human Ig J chain gene | 0.122 |
| 104776 | AA026349 | Hs. 31412 | ESTs | 0.125 |
| 129565 | X77777 | Hs. 198726 | vasoactive intestinal peptide receptor 1 | 0.125 |
| 131272 | AA423884 | Hs. 139033 | paternally expressed gene 3 | 0.127 |
| 105774 | AA348014 | Hs. 23412 | ESTs | 0.128 |
| 134604 | M22995 | Hs. 865 | RAP1A; member of RAS oncogene family | 0.128 |
| 134711 | X04011 | Hs. 88974 | cytochrome b-245; beta polypeptide (chro | 0.128 |
| 129113 | AA147646 | Hs. 108740 | DKFZP586A0522 protein | 0.133 |
| 123995 | D51119 | Hs. 100090 | tetraspan 3 | 0.133 |
| 129168 | T90621 | Hs. 109052 | chromosome 14 open reading frame 2 | 0.133 |
| 123891 | AA621103 | Hs. 99216 | ESTs; Moderately similar to !!!! ALU SUB | 0.135 |
| 132694 | M60830 | Hs. 5509 | ecotropic viral integration site 2B | 0.135 |
| 135342 | W60097 | Hs. 99120 | DEAD/H (Asp-Glu-Ala-Asp/His) box polypep | 0.135 |
| 131510 | AA207114 | Hs. 27842 | ESTs; Weakly similar to similar to 1-acy | 0.137 |
| 133652 | AA287383 | Hs. 7540 | ESTs | 0.137 |
| 134749 | L10955 | Hs. 89485 | carbonic anhydrase IV | 0.139 |
| 106586 | AA456598 | Hs. 256269 | ESTs | 0.139 |
| 106893 | AA489636 | Hs. 25253 | ESTs | 0.139 |
| 101070 | L02785 | Hs. 1650 | down-regulated in adenoma | 0.14 |
| 114293 | Z40718 | Hs. 20196 | adenylate cyclase 9 | 0.14 |
| 113966 | W86600 | Hs. 9842 | ESTs | 0.141 |
| 101185 | L19872 | Hs. 170087 | aryl hydrocarbon receptor | 0.145 |
| 131492 | AA393876 | Hs. 1255 | nuclear receptor subfamily 2; group F; m | 0.145 |
| 133889 | AA099391 | Hs. 211582 | myosin; light polypeptide kinase | 0.145 |
| 120914 | AA377254 | Hs. 97107 | EST | 0.147 |
| 118771 | N74690 | Hs. 50547 | ESTs | 0.149 |
| 105496 | AA256323 | Hs. 25264 | DKFZP434N126 protein | 0.151 |
| 131011 | R41771 | Hs. 22146 | ESTs | 0.153 |
| 106210 | AA428239 | Hs. 10338 | ESTs | 0.154 |
| 114069 | Z38161 | Hs. 197335 | plasma glutamate carboxypeptidase | 0.154 |
| 133011 | AA042990 | Hs. 171921 | sema domain; immunoglobulin domain (Ig); | 0.154 |
| 115967 | AA446887 | Hs. 42911 | ESTs | 0.154 |
| 102571 | U60115 | | Homo sapiens skeletal muscle LIM-protein | 0.155 |
| 100687 | HG3115-HT3291 | | Golli-Mbp (Gb: L18862) | 0.155 |
| 132903 | AA235404 | Hs. 5985 | Homo sapiens clone 25186 mRNA sequence | 0.155 |
| 125832 | AA628600 | Hs. 117587 | ESTs | 0.155 |
| 130064 | T67053 | Hs. 181125 | immunoglobulin lambda gene cluster | 0.157 |
| 123264 | AA491003 | Hs. 99824 | BCE-1 protein | 0.159 |
| 130919 | AA291710 | Hs. 21276 | collagen; type IV; alpha 3 (Goodpasture | 0.159 |
| 103542 | Z11793 | Hs. 3314 | selenoprotein P; plasma; 1 | 0.161 |
| 101478 | M23379 | Hs. 758 | RAS p21 protein activator (GTPase activa | 0.162 |
| 108921 | AA142913 | Hs. 71721 | ESTs | 0.164 |
| 100642 | HG2743-HT3926 | | Caldesmon 1, Alt. Splice 6, Non-Muscle | 0.167 |
| 132109 | AA599801 | Hs. 40098 | ESTs | 0.167 |
| 115719 | AA416997 | Hs. 59622 | ESTs | 0.169 |
| 128915 | C02386 | Hs. 107139 | ESTs | 0.171 |
| 117634 | N36421 | Hs. 107854 | ESTs; Weakly similar to SODIUM-AND CHLO | 0.172 |
| 129462 | D84239 | Hs. 111732 | IgG Fc binding protein | 0.174 |
| 131328 | V01512 | Hs. 25647 | v-fos FBJ murine osteosarcoma viral onco | 0.176 |
| 130343 | AA490262 | Hs. 15485 | ESTs; Weakly similar to APICAL-LIKE PROT | 0.177 |
| 115764 | AA421562 | Hs. 91011 | anterior gradient 2 (Xenepus laevis) hom | 0.177 |
| 122261 | AA436830 | Hs. 98902 | ESTs | 0.179 |
| 106605 | AA457718 | Hs. 21103 | Homo sapiens mRNA; cDNA DKFZp564B076 (fr | 0.179 |
| 109991 | H09813 | Hs. 12896 | KIAA1034 protein | 0.181 |
| 101300 | L40391 | Hs. 6445 | Homo sapiens (clone s153) mRNA fragment | 0.181 |
| 123080 | AA485303 | Hs. 205126 | polymeric immunoglobulin receptor | 0.182 |
| 130016 | AA055811 | Hs. 143131 | transmembrane glycoprotein | 0.186 |
| 122666 | AA455052 | Hs. 99387 | ESTs | 0.188 |
| 105453 | AA252893 | Hs. 9001 | ESTs | 0.189 |
| 108980 | AA151676 | Hs. 33455 | peptidyl arginine deiminase; type II | 0.19 |
| 100248 | D31888 | Hs. 78398 | KIAA0071 protein | 0.192 |
| 130036 | AA195260 | Hs. 206738 | ESTs; Moderately similar to !!!! ALU SUB | 0.192 |
| 110882 | N36001 | Hs. 17348 | ESTs; Weakly similar to !!!! ALU SUBFAMI | 0.193 |
| 131676 | C20785 | Hs. 30514 | ESTs | 0.195 |
| 111029 | N54792 | Hs. 24697 | cytidine monophosphate-N-acetylneuramini | 0.196 |
| 131257 | AA256042 | Hs. 24908 | ESTs | 0.196 |
| 133348 | T23517 | Hs. 7149 | ESTs | 0.196 |
| 133784 | AA214305 | Hs. 76173 | ESTs | 0.196 |
| 113863 | W68388 | Hs. 21288 | ESTs; Weakly similar to KIAA0704 protein | 0.197 |
| 103158 | X67235 | Hs. 118651 | hematopoietically expressed homeobox | 0.198 |
| 102347 | U37518 | Hs. 83429 | tumor necrosis factor (ligand) superfami | 0.2 |
| 111351 | N90223 | Hs. 23392 | ESTs | 0.2 |
| 123495 | AA599850 | Hs. 106747 | ESTs; Weakly similar to similar to BPTV | 0.2 |
| 123802 | AA620448 | Hs. 61408 | Homo sapiens clone 24760 mRNA sequence | 0.2 |
| 129243 | H88033 | Hs. 109727 | KIAA0733 protein | 0.2 |
| 130219 | R77539 | Hs. 15285 | ESTs | 0.2 |
| 131171 | H04644 | Hs. 167619 | ESTs; Weakly similar to !!!! ALU SUBFAMI | 0.2 |
| 133746 | U44378 | Hs. 75862 | MAD (mothers against decapentaplegic; Dr | 0.2 |
| 116459 | AA621399 | Hs. 64193 | ESTs | 0.201 |
| 109613 | F03031 | Hs. 27519 | ESTs | 0.202 |
| 133435 | T23983 | Hs. 7365 | ESTs | 0.202 |
| 103002 | X52001 | Hs. 1408 | endothelin 3 | 0.204 |
| 125153 | W38294 | | Accession not listed in Genbank | 0.204 |
| 131919 | AA121266 | Hs. 34641 | ESTs | 0.204 |
| 100749 | HG3521-HT3715 | | Ras-Related Protein Rap1b | 0.205 |
| 105085 | AA147537 | Hs. 4811 | ESTs | 0.208 |
| 124571 | N67470 | Hs. 173074 | DKFZP564O1863 protein | 0.21 |
| 129519 | AA298786 | Hs. 112242 | ESTs | 0.21 |
| 116724 | F13665 | Hs. 65641 | ESTs | 0.21 |
| 132932 | T15482 | Hs. 6093 | ESTs | 0.21 |
| 113803 | W42789 | Hs. 31446 | ESTs | 0.211 |
| 110792 | N24899 | Hs. 6630 | ESTs | 0.212 |
| 105178 | AA187490 | Hs. 21941 | ESTs | 0.212 |
| 107295 | T34527 | Hs. 80120 | UDP-N-acetyl-alpha-D-galactosamine: polyp | 0.212 |
| 115262 | AA279112 | Hs. 88594 | ESTs | 0.213 |
| 115839 | AA429038 | Hs. 40541 | ESTs | 0.213 |
| 103211 | X73079 | Hs. 205126 | polymeric immunoglobulin receptor | 0.214 |
| 108604 | AA099820 | Hs. 49696 | ESTs | 0.215 |
| 105173 | AA182030 | Hs. 8364 | ESTs | 0.217 |
| 108539 | AA084677 | Hs. 54558 | ESTs; Weakly similar to protein B [H.sap | 0.217 |
| 109984 | H09594 | Hs. 10299 | ESTs | 0.217 |
| 133536 | Y00264 | Hs. 177486 | amyloid beta (A4) precursor protein (pro | 0.217 |
| 129965 | T71333 | Hs. 13854 | ESTs | 0.219 |
| 114542 | AA055768 | Hs. 122576 | ESTs | 0.219 |
| 132982 | L02326 | Hs. 198118 | immunoglobulin lambda-like polypeptide 2 | 0.22 |
| 101809 | M86849 | | Homo sapiens connexin 26 (GJB2) mRNA, co | 0.222 |
| 105795 | AA369245 | Hs. 17448 | ESTs; Weakly similar to !!!! ALU SUBFAMI | 0.222 |
| 132119 | H99211 | Hs. 40334 | ESTs | 0.222 |
| 132733 | R25385 | Hs. 123654 | KIAA0824 protein | 0.222 |
| 109415 | AA227219 | Hs. 110826 | trinucleotide repeat containing 9 | 0.222 |
| 113083 | T40530 | Hs. 8241 | ESTs; Weakly similar to heat shock prote | 0.223 |
| 107053 | AA600147 | Hs. 5741 | ESTs; Weakly similar to NADH-cytochrome | 0.224 |
| 103653 | Z70295 | Hs. 32966 | guanylate cyclase activator 2B (uroguany | 0.225 |
| 104613 | AA001049 | Hs. 24713 | Homo sapiens mRNA; cDNA DKFZp586G0123 (f | 0.225 |
| 126180 | R18070 | Hs. 3712 | ubiquinol-cytochrome c reductase; Rieske | 0.227 |
| 132015 | D11900 | Hs. 3731 | ESTs | 0.227 |
| 130616 | AA233763 | Hs. 16726 | Homo sapiens mRNA; cDNA DKFZp564A132 (fr | 0.227 |
| 132883 | AA047151 | Hs. 5897 | Homo sapiens mRNA; cDNA DKFZp586P1622 (f | 0.23 |
| 123169 | AA488892 | Hs. 104472 | ESTs; Weakly similar to Gag-Pol polyprot | 0.233 |
| 115187 | AA261805 | Hs. 44021 | ESTs | 0.234 |
| 116787 | H28581 | Hs. 15641 | ESTs | 0.234 |
| 113195 | T57112 | | yc20g11.s1 Stratagene lung (#937210) Hom | 0.235 |
| 130707 | W45457 | Hs. 203559 | ESTs | 0.235 |
| 124803 | R45480 | Hs. 164866 | cyclin K | 0.235 |
| 116844 | H64938 | Hs. 38331 | ESTs | 0.235 |
| 102759 | U81607 | Hs. 788 | A kinase (PRKA) anchor protein (gravin) | 0.238 |
| 130584 | AA009839 | Hs. 180841 | tumor necrosis factor receptor superfami | 0.238 |
| 133240 | D31161 | Hs. 68613 | ESTs | 0.238 |
| 132952 | AA425154 | Hs. 61426 | ESTs | 0.239 |
| 132720 | Z69881 | Hs. 5541 | ATPase; Ca++ transporting; ubiquitous | 0.24 |
| 131734 | D62965 | Hs. 31297 | ESTs | 0.24 |
| 111890 | R38678 | Hs. 12365 | ESTs | 0.241 |
| 102325 | U35139 | Hs. 50130 | necdin (mouse) homolog | 0.244 |
| 104968 | AA084602 | Hs. 29669 | ESTs | 0.244 |
| 105674 | AA284755 | Hs. 214742 | CDW52 antigen (CAMPATH-1 antigen) | 0.244 |
| 120519 | AA258585 | Hs. 129887 | cadherin 19 (NOTE: redefinition of symbo | 0.244 |
| 134675 | AA250745 | Hs. 87773 | protein kinase; cAMP-dependent; catalyti | 0.244 |
| 130642 | M63438 | Hs. 156110 | Immunoglobulin kappa variable 1D-8 | 0.245 |
| 134418 | R78190 | Hs. 82933 | ESTs; Weakly similar to cDNA EST EMBL: T0 | 0.245 |
| 115137 | AA257976 | Hs. 56156 | ESTs | 0.245 |
| 131713 | X57809 | Hs. 181125 | immunoglobulin lambda gene cluster | 0.246 |
| 108931 | AA147186 | Hs. 250746 | ESTs | 0.246 |
| 106609 | AA458652 | Hs. 32181 | ESTs | 0.248 |
| 115559 | AA393810 | Hs. 41067 | ESTs | 0.25 |
| 133985 | L34657 | Hs. 78146 | platelet/endothelial cell adhesion molec | 0.25 |
| 134088 | D43636 | Hs. 79025 | KIAA0096 protein | 0.25 |
| 134487 | R38185 | Hs. 83954 | Homo sapiens unknown mRNA | 0.25 |

Pkey: Unique Eos probeset identifier number
ExAccn: Exemplar Accession number, Genbank accession number
UnigeneID: Unigene number
Unigene Title: Unigene gene title

TABLE 15
I chip - Met vs Normal query - up in Mets

| Pkey | Ex_Accn | UniG_ID | Title | Ratio Met/Normal |
|---|---|---|---|---|
| 319379 | T91443 | Hs. 193963 | ESTs | 18.71 |
| 321920 | N63915 | | EST cluster (not in UniGene) | 11.9 |
| 314522 | AI732331 | Hs. 187750 | ESTs; Moderately similar to !!!! ALU CLA | 7.23 |
| 315720 | AW291875 | Hs. 163900 | ESTs | 6.06 |
| 308010 | AI439190 | Hs. 181165 | eukaryotic translation elongation factor | 5.76 |
| 313774 | AW136836 | Hs. 144583 | ESTs | 5.01 |
| 300734 | AW205197 | Hs. 240951 | ESTs | 3.98 |
| 337895 | | | CH22_EM: AC005500.GENSCAN.56-2 | 3.98 |
| 312339 | AA524394 | | EST cluster (not in UniGene) | 3.66 |
| 331644 | T99544 | Hs. 173734 | ESTs; Weakly similar to !!!! ALU CLASS B | 3.53 |
| 324643 | AI436356 | Hs. 130729 | ESTs | 3.52 |
| 324302 | AA543008 | Hs. 136806 | ESTs; Weakly similar to !!!! ALU SUBFAMI | 3.41 |
| 314912 | AI431345 | Hs. 161784 | ESTs | 3.33 |
| 319403 | T98413 | | EST cluster (not in UniGene) | 3.32 |
| 308676 | AI761036 | | EST singleton (not in UniGene) with exon | 3.27 |
| 331858 | AA421163 | Hs. 163848 | ESTs | 3.22 |
| 315178 | AW362945 | Hs. 162459 | ESTs | 3.21 |
| 321354 | AA078493 | | EST cluster (not in UniGene) | 3.18 |
| 337898 | | | CH22_EM: AC005500.GENSCAN.56-5 | 3.16 |
| 322682 | AI110679 | | EST cluster (not in UniGene) | 3.15 |
| 313197 | AI738851 | Hs. 222487 | ESTs | 3.1 |
| 308991 | AI879831 | | EST singleton (not in UniGene) with exon | 3.08 |
| 310016 | AW449612 | Hs. 152475 | ESTs | 3.05 |

Pkey: Unique Eos probeset identifier number
ExAccn: Exemplar Accession number, Genbank accession number
UnigeneID: Unigene number
Unigene Title: Unigene gene title

TABLE 16
I chip - Met vs Normal query - down in Mets

| Pkey | Ex_Accn | UniG_ID | title | Ratio Met/Normal |
|---|---|---|---|---|
| 303041 | AF127035 | | EST cluster (not in UniGene) with exon h | 0.02 |
| 302360 | AJ010901 | Hs. 198267 | mucin 4; tracheobronchial | 0.03 |
| 301948 | AA344647 | Hs. 116724 | aldo-keto reductase family 1; member B11 | 0.03 |
| 336091 | | | CH22_FGENES.689_3 | 0.04 |
| 333657 | | | CH22_FGENES.241_2 | 0.04 |
| 333658 | | | CH22_FGENES.241_4 | 0.04 |
| 333737 | | | CH22_FGENES.261_1 | 0.05 |
| 333656 | | | CH22_FGENES.240_4 | 0.05 |
| 302347 | AF039400 | Hs. 194659 | chloride channel; calcium activated; fam | 0.06 |
| 336084 | | | CH22_FGENES.688_13 | 0.06 |
| 330385 | AA449749 | Hs. 31386 | ESTs; Highly similar to secreted apoptos | 0.06 |
| 304487 | AA434241 | | EST singleton (not in UniGene) with exon | 0.07 |
| 302292 | AF067797 | | EST cluster (not in UniGene) with exon h | 0.07 |
| 334030 | | | CH22_FGENES.320_2 | 0.07 |
| 332859 | | | CH22_FGENES.27_2 | 0.07 |
| 333654 | | | CH22_FGENES.240_2 | 0.07 |
| 303270 | AL120518 | Hs. 105352 | ESTs | 0.08 |
| 320352 | Y13323 | Hs. 145296 | disintegrin protease | 0.08 |
| 333637 | | | CH22_FGENES.229_2 | 0.08 |
| 324094 | AA382603 | | EST cluster (not in UniGene) | 0.08 |
| 320590 | U67058 | Hs. 168102 | Human proteinase activated receptor-2 mR | 0.08 |
| 330622 | X63597 | Hs. 2996 | sucrase-isomaltase | 0.08 |
| 331441 | H75860 | Hs. 39720 | ESTs | 0.08 |
| 308601 | AI719930 | | EST singleton (not in UniGene) wilh exon | 0.09 |
| 323770 | AA722425 | | EST cluster (not in UniGene) | 0.09 |
| 335188 | | | CH22_FGENES.507_3 | 0.09 |
| 333730 | | | CH22_FGENES.258_1 | 0.09 |
| 304480 | AA430373 | | EST singleton (not in UniGene) with exon | 0.09 |
| 336081 | | | CH22_FGENES.688_10 | 0.1 |
| 332071 | AA598594 | Hs. 112475 | ESTs | 0.1 |
| 318538 | N28625 | Hs. 74034 | caveolin 1; caveolae protein; 22 kD | 0.1 |
| 311331 | AI679622 | Hs. 32225 | immunoglobulin alpha 1 | 0.1 |
| 319668 | NM_002731 | | EST cluster (not in UniGene) | 0.11 |
| 332567 | N23730 | Hs. 25647 | v-fos FBJ murine osteosarcoma viral onco | 0.11 |
| 319395 | AW062570 | Hs. 13809 | ESTs | 0.11 |
| 315594 | AI983437 | Hs. 155145 | ESTs | 0.11 |
| 321539 | N98619 | Hs. 62461 | ARP2 (actin-related protein 2; yeast) ho | 0.12 |
| 333647 | | | CH22_FGENES.235_2 | 0.12 |
| 333588 | | | CH22_FGENES.206_2 | 0.12 |
| 321286 | AI380940 | | EST cluster (not in UniGene) | 0.12 |
| 320727 | U96044 | | EST cluster (not in UniGene) | 0.13 |
| 335687 | | | CH22_FGENES.596_2 | 0.13 |
| 324611 | AA743462 | Hs. 165337 | ESTs | 0.14 |
| 335115 | | | CH22_FGENES.496_2 | 0.14 |
| 324660 | AA541644 | Hs. 186044 | ESTs | 0.14 |
| 337951 | | | CH22_EM: AC005500.GENSCAN.94-1 | 0.14 |
| 302332 | AI833168 | Hs. 184507 | Homo sapiens Chromosome 16 BAC clone CIT | 0.14 |
| 300921 | AW293224 | Hs. 232165 | ESTs | 0.14 |
| 333646 | | | CH22_FGENES.234_2 | 0.14 |
| 335116 | | | CH22_FGENES.496_3 | 0.14 |
| 320211 | AL039402 | Hs. 125783 | DEME-6 protein | 0.15 |
| 336092 | | | CH22_FGENES.689_6 | 0.15 |
| 330673 | D57823 | Hs. 92962 | Sec23 (S. cerevisiae) homolog A | 0.16 |
| 303042 | AF129532 | | EST cluster (not in UniGene) with exon h | 0.16 |
| 337954 | | | CH22_EM: AC005500.GENSCAN.96-3 | 0.16 |
| 336645 | | | CH22_FGENES.26-1 | 0.16 |
| 335651 | | | CH22_FGENES.590_2 | 0.16 |
| 314499 | AL044570 | Hs. 147975 | ESTs | 0.17 |
| 336124 | | | CH22_FGENES.701_9 | 0.17 |
| 315199 | AA877996 | Hs. 125376 | ESTs | 0.17 |
| 324525 | AW044647 | Hs. 196284 | ESTs | 0.17 |
| 320825 | NM_004751 | | EST cluster (not in UniGene) | 0.18 |
| 302049 | AA377072 | Hs. 129792 | Homo sapiens Chromosome 16 BAC clone CIT | 0.18 |
| 336083 | | | CH22_FGENES.688_12 | 0.18 |
| 333653 | | | CH22_FGENES.239_2 | 0.18 |
| 323243 | W44372 | | EST cluster (not in UniGene) | 0.19 |
| 316610 | AW087973 | Hs. 126731 | ESTs | 0.19 |
| 315033 | AI493046 | Hs. 146133 | ESTs | 0.19 |
| 330551 | U39840 | Hs. 105440 | hepatocyte nuclear factor 3; alpha | 0.19 |
| 333642 | | | CH22_FGENES.231_2 | 0.19 |
| 301281 | AA843986 | Hs. 190586 | ESTs | 0.2 |
| 333626 | | | CH22_FGENES.224_2 | 0.21 |
| 303792 | C75094 | Hs. 199839 | ESTs; Highly similar to NG22 [H. sapiens] | 0.21 |
| 332325 | T79428 | Hs. 191264 | ESTs | 0.21 |
| 321223 | AA431366 | | EST cluster (not in UniGene) | 0.21 |
| 333635 | | | CH22_FGENES.228_2 | 0.22 |
| 314645 | AI808999 | Hs. 207570 | ESTs | 0.22 |
| 322929 | AI365585 | Hs. 146246 | ESTs | 0.22 |
| 324718 | AI557019 | Hs. 116467 | ESTs | 0.22 |
| 335652 | | | CH22_FGENES.590_3 | 0.22 |
| 307783 | AI347274 | | EST singleton (not in UniGene) with exon | 0.22 |
| 331344 | AA357927 | Hs. 70208 | ESTs | 0.22 |
| 336088 | | | CH22_FGENES.688_17 | 0.23 |
| 320802 | D83824 | Hs. 185055 | BENE protein | 0.23 |
| 335692 | | | CH22_FGENES.596_7 | 0.23 |
| 333593 | | | CH22_FGENES.210_2 | 0.23 |
| 335667 | | | CH22_FGENES.590_18 | 0.24 |
| 314853 | AA729232 | Hs. 153279 | ESTs | 0.24 |
| 320244 | AA296922 | Hs. 129778 | gastrointestinal peptide | 0.24 |
| 300601 | AI762130 | Hs. 165619 | ESTs | 0.24 |
| 305080 | AA641485 | | EST singleton (not in UniGene) with exon | 0.25 |
| 335189 | | | CH22_FGENES.507_4 | 0.25 |

Pkey: Unique Eos probeset identifier number
ExAccn: Exemplar Accession number, Genbank accession number
UnigeneID: Unigene number
Unigene Title: Unigene gene title

TABLE 17
B survivor vs Mets - Up in B survivor

| Pkey | Ex Accn | UniG_ID | Complete Title | Ratio BS/Met |
|---|---|---|---|---|
| 101006 | J04132 | Hs. 97087 | CD3Z antigen; zeta polypeptide (TiT3 com | 7.28 |
| 114173 | Z39050 | Hs. 21963 | ESTs | 6.13 |
| 130284 | X82206 | Hs. 153961 | ARP1 (actin-related protein 1; yeast) ho | 5.77 |
| 100787 | HG3872-HT4142 | | Immunoglobulin Gamma Heavy Chain, V(6)Djc Regions (Gb: U13200) | 5.63 |
| 132461 | AA405775 | Hs. 49005 | hypothetical protein | 5.62 |
| 133806 | M12759 | Hs. 76325 | Human Ig J chain gene | 5.46 |
| 133747 | D86972 | Hs. 75863 | KIAA0218 gene product | 5.45 |
| 123328 | AA496968 | Hs. 105403 | EST | 5.28 |
| 132671 | X76302 | Hs. 54649 | putative nucleic acid binding protein RY | 5.25 |
| 132018 | AA293194 | Hs. 3737 | ESTs | 5.22 |
| 100186 | D17516 | Hs. 4748 | adenylate cyclase activating polypeptide | 5.14 |
| 107155 | AA621202 | Hs. 7946 | DKFZP586D1519 protein | 5.1 |
| 103566 | Z22555 | Hs. 180616 | CD36 antigen (collagen type I receptor; | 5.06 |
| 113355 | T79203 | Hs. 14480 | ESTs | 4.99 |
| 129040 | U38864 | Hs. 108139 | zinc finger protein 212 | 4.96 |
| 130214 | H78003 | Hs. 15266 | ESTs | 4.93 |
| 129550 | AA480991 | Hs. 113025 | ESTs | 4.92 |
| 129704 | W81301 | Hs. 12064 | ubiquitin specific protease 22 | 4.91 |
| 116425 | AA609574 | Hs. 51483 | ESTs | 4.77 |
| 105166 | AA179787 | Hs. 30570 | polyglutamine binding protein 1 | 4.65 |
| 118765 | N74442 | Hs. 183696 | ESTs | 4.6 |
| 108999 | AA156064 | Hs. 72115 | ESTs | 4.57 |
| 112756 | R93908 | Hs. 35258 | ESTs | 4.54 |
| 111655 | R16884 | Hs. 187462 | ESTs | 4.48 |
| 119392 | T90672 | Hs. 238859 | ESTs | 4.42 |
| 131957 | AA609008 | Hs. 183232 | ESTs | 4.41 |
| 129275 | D82061 | Hs. 109993 | Ke6 gene; mouse; human homolog of | 4.4 |
| 113634 | T95085 | Hs. 125182 | ESTs | 4.4 |
| 127187 | AA297138 | Hs. 207422 | ESTs | 4.32 |
| 101147 | L13266 | Hs. 105 | glutamate receptor; ionotropic; N-methyl | 4.3 |
| 134901 | S78873 | Hs. 90875 | RAB interacting factor | 4.26 |
| 100896 | HG4593-HT4998 | | Sodium Channel 1 | 4.24 |
| 100687 | HG3115-HT3291 | | Golli-Mbp (Gb: L18862) | 4.21 |
| 129758 | AA599552 | Hs. 183770 | Homo sapiens mRNA; cDNA DKFZp566P2346 (f | 4.19 |
| 105440 | AA252243 | Hs. 22851 | ESTs | 4.16 |
| 131551 | AA127867 | Hs. 28608 | ESTs | 4.15 |
| 113761 | T99373 | Hs. 189786 | ESTs | 4.09 |
| 105897 | AA401091 | | ESTs | 4.07 |
| 129495 | AA382529 | Hs. 239676 | ESTs | 4.06 |
| 103436 | X98206 | | H. sapiens mRNA for UV-B repressed sequen | 4.03 |
| 104673 | AA007633 | Hs. 20010 | ESTs | 4.03 |
| 128886 | L36720 | Hs. 106880 | bystin-like | 4.02 |
| 100702 | HG3236-HT3413 | | Neurofibromatosis 2 Tumor Suppressor (Gb: L27065) | 3.99 |
| 123547 | AA608820 | Hs. 124085 | KIAA0921 protein | 3.98 |
| 134877 | AA455241 | Hs. 90527 | ESTs | 3.97 |
| 123650 | AA609332 | Hs. 180696 | ESTs | 3.94 |
| 106482 | AA451672 | Hs. 108824 | ESTs; Weakly similar to cDNA EST yk415c1 | 3.94 |
| 101909 | S69265 | | Homo sapiens mRNA for PLE21 protein; com | 3.93 |
| 108390 | AA075070 | | zm86b6.s1 Stratagene ovarian cancer (#93 | 3.93 |
| | | | LYMPHOCYTE ANTIGEN LY-6A.2/LY-6E.1 PREC | |
| 135403 | U06643 | Hs. 99923 | lectin; galactoside-binding; soluble; 7 | 3.89 |
| 121038 | AA398536 | Hs. 97365 | ESTs | 3.88 |
| 128496 | T83496 | Hs. 100610 | ESTs | 3.86 |
| 108785 | AA128946 | | ESTs | 3.86 |
| 119838 | W79499 | Hs. 58580 | ESTs | 3.85 |
| 130109 | L12060 | Hs. 1497 | retinoic acid receptor; gamma | 3.84 |
| 134538 | U79288 | Hs. 85053 | KIAA0513 gene product | 3.83 |
| 110310 | H38209 | Hs. 32728 | EST | 3.81 |
| 110433 | H49425 | Hs. 32992 | ESTs | 3.78 |
| 111834 | R36138 | Hs. 152458 | ESTs | 3.76 |
| 130903 | N27086 | Hs. 21068 | ESTs | 3.74 |
| 105142 | AA164851 | Hs. 15380 | ESTs; Weakly similar to HERV-E envelope | 3.73 |
| 130248 | U84569 | Hs. 153452 | chromosome 21 open reading frame 2 | 3.73 |
| 130645 | AA020942 | Hs. 17200 | STAM-like protein containing SH3 and ITA | 3.73 |
| 123378 | AA521043 | Hs. 185832 | ESTs | 3.73 |
| 103985 | AA313880 | | EST185737 Colon carcinoma (HCC) cell in | 3.73 |
| 112397 | R60822 | Hs. 26805 | EST | 3.72 |
| 100980 | J03069 | Hs. 72931 | v-myc avian myelocytomatosis viral oncog | 3.72 |
| 102609 | U64863 | Hs. 158297 | programmed cell death 1 | 3.7 |
| 108974 | AA151402 | Hs. 46531 | ESTs | 3.7 |
| 130192 | Y12661 | Hs. 171014 | VGF nerve growth factor inducible | 3.69 |
| 131318 | X51699 | Hs. 2558 | bone gamma-carboxyglutamate (gla) protei | 3.68 |
| 113759 | T99364 | Hs. 16074 | Homo sapiens mRNA; cDNA DKFZp564\|153 (fr | 3.66 |
| 133712 | L19267 | Hs. 198836 | dystrophia myotonica-containing WD repea | 3.65 |
| 134229 | R15108 | Hs. 8037 | ESTs | 3.65 |
| 134241 | AA300265 | Hs. 80540 | KIAA0195 gene product | 3.65 |
| 124699 | R06413 | Hs. 112278 | arrestin; beta 1 | 3.62 |
| 107343 | U03115 | Hs. 103945 | Human V beta T-cell receptor (TCRBV) gen | 3.62 |
| 128511 | AA425636 | Hs. 10082 | potassium intermediate/small conductance | 3.62 |
| 105466 | AA253412 | Hs. 21489 | ESTs | 3.61 |
| 131377 | R41389 | Hs. 26159 | ESTs | 3.6 |
| 119135 | R49548 | Hs. 169681 | death effector domain-containing | 3.6 |
| 132982 | L02326 | Hs. 198118 | immunoglobulin lambda-like polypeptide 2 | 3.59 |
| 128514 | H84261 | Hs. 100843 | ESTs; Weakly similar to similar to GTP-b | 3.56 |
| 102396 | U41804 | Hs. 54411 | putative T1/ST2 receptor binding protein | 3.55 |
| 134945 | R50247 | Hs. 91600 | ESTs | 3.55 |
| 134913 | X60483 | Hs. 91031 | H4 histone family; member D | 3.54 |
| 102053 | U07664 | Hs. 37035 | homeo box HB9 | 3.52 |
| 121569 | AA412686 | Hs. 97955 | ESTs | 3.52 |
| 132560 | AA005315 | Hs. 204524 | ESTs; Weakly similar to KIAA0747 protein | 3.51 |
| 118456 | N66580 | Hs. 161496 | EST; Weakly similarto HC1 ORF [M.muscul | 3.51 |
| 111518 | R08160 | Hs. 222529 | ESTs; Weakly similar to !!!! ALU SUBFAMI | 3.51 |
| 116795 | H38858 | Hs. 251783 | EST | 3.5 |
| 130377 | AA378316 | Hs. 155182 | KIAA1036 protein | 3.5 |
| 121774 | AA421756 | Hs. 98361 | ESTs | 3.49 |
| 123413 | AA521448 | Hs. 103845 | ESTs | 3.49 |
| 133798 | AA444115 | Hs. 76277 | ESTs; Weakly similar to salivary proline | 3.49 |
| 135183 | X93996 | Hs. 239663 | myeloid/lymphoid or mixed-lineage leukem | 3.48 |
| 132479 | AA477715 | Hs. 4953 | golgi autoantigen; golgin subfamily a; 3 | 3.47 |
| 117191 | H99394 | Hs. 40339 | EST | 3.47 |
| 130942 | X87852 | Hs. 21432 | H. sapiens mRNA for SEX gene | 3.46 |
| 130700 | D55696 | Hs. 18069 | protease; cysteine; 1 (legumain) | 3.43 |
| 131301 | T17386 | Hs. 164501 | ESTs | 3.43 |
| 100818 | HG4018-HT4288 | | Opioid-Binding Cell Adhesion Molecule | 3.43 |
| 103393 | X94612 | Hs. 41749 | protein kinase; cGMP-dependent type II | 3.43 |
| 131337 | AA228116 | Hs. 170204 | KIAA0551 protein | 3.42 |
| 133403 | X68688 | Hs. 72991 | zinc finger protein 33b (KOX 31) | 3.42 |
| 124728 | R16231 | Hs. 106620 | Homo sapiens clone 23950 mRNA sequence | 3.41 |
| 123168 | AA488881 | Hs. 105218 | EST | 3.39 |
| 123324 | AA496932 | Hs. 105399 | KIAA0809 protein | 3.38 |
| 106947 | AA496685 | Hs. 37936 | suppressor of variegation 3-9 (Drosophil | 3.38 |
| 116717 | F11065 | Hs. 79363 | ESTs | 3.36 |
| 102794 | U88629 | Hs. 173334 | ELL-RELATED RNA POLYMERASE II; ELONGATIO | 3.34 |
| 117503 | N31963 | Hs. 44286 | ESTs | 3.33 |
| 112220 | R50295 | Hs. 25703 | ESTs | 3.33 |
| 106340 | AA441792 | Hs. 22857 | chord domain-containing protein 1 | 3.33 |
| 106308 | AA436186 | Hs. 30662 | ESTs | 3.32 |
| 130894 | D16105 | Hs. 210 | leukocyte tyrosine kinase | 3.31 |
| 120039 | W92548 | Hs. 94985 | ESTs | 3.31 |
| 131428 | U17838 | Hs. 26719 | PR domain containing 2; with ZNF domain | 3.3 |
| 113285 | T66830 | Hs. 182712 | ESTs | 3.3 |
| 109458 | AA232648 | Hs. 87068 | ESTs | 3.29 |
| 132134 | AA242904 | Hs. 40637 | proline-rich Gla (G-carboxyglutamic acid | 3.29 |
| 118964 | N93330 | Hs. 54937 | Homo sapiens clone 24722 unknown mRNA; p | 3.29 |
| 127621 | AI218205 | Hs. 116204 | ESTs | 3.29 |
| 135149 | U40002 | Hs. 95351 | lipase; hormone-sensitive | 3.28 |
| 114371 | Z41835 | Hs. 27810 | ESTs | 3.28 |
| 130043 | AA055404 | Hs. 193953 | ESTs; Weakly similar to !!!! ALU SUBFAMI | 3.27 |
| 121347 | AA405181 | Hs. 97972 | ESTs | 3.25 |
| 105754 | AA302657 | Hs. 192028 | ESTs | 3.25 |
| 121327 | AA404286 | Hs. 173125 | peptidylprolyl isomerase F (cyclophilin | 3.25 |
| 111204 | N68295 | Hs. 37982 | ESTs | 3.25 |
| 120949 | AA397830 | Hs. 98347 | ESTs; Weakly similar to GLIOMA PATHOGENE | 3.25 |
| 130024 | U15197 | Hs. 241560 | Human histo-blood group ABO protein mRNA | 3.24 |
| 125005 | T61449 | Hs. 193727 | ESTs | 3.24 |
| 121067 | AA398662 | Hs. 97302 | ESTs | 3.24 |
| 120996 | AA398281 | Hs. 143684 | ESTs | 3.23 |
| 117101 | H94043 | Hs. 24341 | DKFZP586\|1419 protein | 3.23 |
| 130708 | U40490 | Hs. 18136 | nicotinamide nucleotide transhydrogenase | 3.23 |
| 130270 | L40399 | Hs. 153820 | hypothetical protein | 3.22 |
| 131605 | AA256220 | Hs. 29383 | ESTs | 3.22 |
| 100854 | HG4194-HT4464 | | Sodium/Hydrogen Exchanger 5 | 3.22 |
| 123026 | AA481072 | Hs. 99743 | ESTs | 3.21 |
| 108328 | AA070204 | | zm68b3.s1 Stratagene neuroepithelium (#9 | 3.2 |
| 104259 | AF007833 | Hs. 159265 | Homo sapiens kruppel-related zinc finger | 3.2 |
| 133711 | J04130 | Hs. 75703 | small inducible cytokine A4 (homologous | 3.2 |
| 112261 | R52145 | Hs. 25894 | ESTs; Highly similar to hypothetical pro | 3.19 |
| 119529 | W38053 | | Accession not listed in Genbank | 3.19 |
| 122386 | AA446221 | Hs. 6092 | F-box protein containing leucine-rich re | 3.19 |
| 109157 | AA179161 | Hs. 73562 | ESTs | 3.19 |
| 119903 | W85707 | Hs. 75936 | erythrocyte membrane protein band 4.9 (d | 3.18 |
| 127452 | AA491317 | | aa65c01.r1 NCI_CGAP_GCB1 Homo sapiens cD | 3.18 |
| 124229 | H62793 | Hs. 221892 | ESTs | 3.18 |
| 129221 | AA417126 | Hs. 109571 | translocase of inner mitochondrial membr | 3.17 |
| 133185 | AA481404 | Hs. 6686 | ESTs | 3.16 |
| 121479 | AA411911 | Hs. 98110 | ESTs | 3.16 |
| 133872 | T79868 | Hs. 180903 | hypothetical protein | 3.16 |
| 132504 | U12897 | Hs. 5022 | imprinted in Prader-Willi syndrome | 3.16 |
| 103089 | X60382 | Hs. 179729 | collagen; type X; alpha 1 (Schmid metaph | 3.15 |
| 129654 | AA019943 | Hs. 118463 | H. sapiens mRNA for unknown liver orphan | 3.15 |
| 117295 | N22360 | Hs. 43153 | ESTs | 3.15 |
| 107349 | U48224 | Hs. 158321 | beaded filament structural protein 2; ph | 3.14 |
| 103451 | X99459 | Hs. 154782 | adaptor-related protein complex 3; sigma | 3.14 |
| 114854 | AA235056 | Hs. 120244 | ESTs | 3.14 |
| 121044 | AA398551 | Hs. 97374 | ESTs | 3.13 |
| 128582 | U22963 | Hs. 101840 | major histocompatibility complex; class | 3.13 |
| 112598 | R78565 | Hs. 138395 | EST | 3.13 |
| 113170 | T54342 | Hs. 222506 | ESTs | 3.13 |
| 111714 | R23146 | Hs. 23466 | ESTs | 3.13 |
| 111809 | R33616 | Hs. 24688 | EST | 3.12 |
| 115249 | AA278961 | Hs. 71124 | ESTs | 3.11 |
| 103228 | X75546 | Hs. 230 | fibromodulin | 3.11 |
| 129944 | L00389 | Hs. 1361 | cytochrome P450; subfamily I (aromatic c | 3.11 |
| 107927 | AA028915 | Hs. 237709 | EST | 3.11 |
| 130297 | H94949 | Hs. 171955 | trophinin-assisting protein (tastin) | 3.1 |
| 125742 | H81181 | Hs. 183654 | ESTs; Weakly similar to unknown [S.cerev | 3.1 |
| 134802 | L35548 | Hs. 89709 | glutamate-cysteine ligase (gamma-glutamy | 3.1 |
| 112560 | R72293 | Hs. 6179 | Homo sapiens mRNA; cDNA DKFZp586K2322 (f | 3.1 |
| 129266 | AA343881 | Hs. 209061 | sudD (suppressor of bimD6; Aspergillus n | 3.09 |
| 126982 | AA211419 | | small inducible cytokine A5 (RANTES) | 3.09 |
| 131594 | H29723 | Hs. 29261 | ESTs; Weakly similar to serine protease | 3.08 |
| 134910 | AA431320 | Hs. 9100 | ESTs | 3.08 |
| 103505 | Y09912 | Hs. 33102 | transcription factor AP-2 beta (activati | 3.08 |
| 110525 | H57330 | Hs. 37430 | EST | 3.07 |
| 123276 | AA491270 | Hs. 187946 | ESTs | 3.06 |
| 130519 | H91819 | Hs. 10669 | ESTs; Moderately similar to KIAA0400 [H. | 3.06 |
| 126621 | AA192638 | | zq01h08.r1 Stratagene muscle 937209 Homo | 3.05 |
| 134327 | AF006041 | Hs. 178743 | death-associated protein 6 | 3.04 |
| 103513 | Y10209 | | H. sapiens mRNA for CD3L protein | 3.04 |
| 131243 | R16667 | Hs. 24752 | spectrin SH3 domain binding protein 1 | 3.04 |
| 115187 | AA261805 | Hs. 44021 | ESTs | 3.04 |
| 107543 | Z43703 | Hs. 4552 | Homo sapiens HRIHFB2157 mRNA; partial cd | 3.04 |
| 134051 | S67070 | Hs. 78846 | heat shock 27 kD protein 2 | 3.04 |
| 113461 | T86737 | Hs. 193536 | ESTs | 3.03 |
| 130490 | X57522 | Hs. 158164 | ATP-binding cassette; sub-family B (MDR/ | 3.03 |
| 128843 | AA234141 | Hs. 203004 | katanin p80 (WD40-containing) subunit B | 3.03 |
| 100941 | HG862-HT862 | | Transition Protein 2 | 3.03 |
| 122268 | AA436855 | Hs. 178202 | ESTs | 3.02 |
| 107425 | W26719 | Hs. 30204 | ESTs | 3.02 |
| 130930 | U19261 | | TNF receptor-associated factor 1 | 3.02 |
| 132958 | W90398 | Hs. 6147 | KIAA1075 protein | 3.02 |
| 100973 | J02888 | Hs. 73956 | NAD(P)H menadione oxidoreductase 2; diox | 3.01 |
| 104924 | AA058532 | Hs. 28774 | ESTs | 3.01 |
| 129998 | Y10055 | Hs. 162808 | phosphoinositide-3-kinase; catalytic; de | 3.01 |
| 130023 | X13401 | Hs. 239600 | calmodulin-like 3 | 3.01 |
| 129536 | M33493 | Hs. 184504 | tryptase; alpha | 3 |
| 112015 | R42836 | Hs. 23198 | ESTs | 3 |
| 103036 | X54925 | Hs. 83169 | matrix metalloproteinase 1 (interstitial | 2.99 |
| 100756 | HG3565-HT3768 | | Zinc Finger Protein (Gb: M88357) | 2.99 |
| 103425 | X97301 | | H. sapiens mRNA for Ptg-11 protein | 2.99 |
| 118291 | N63076 | Hs. 138745 | EST | 2.98 |
| 125877 | H15229 | | ym30g04.r1 Soares infant brain 1NIB Homo | 2.98 |
| | | | repetitive element;, mRNA sequence. | |
| 101371 | M13232 | Hs. 36989 | coagulation factor VII (serum prothrombi | 2.98 |
| 102958 | X15675 | Hs. 93174 | Human endogenous retrovirus pHE.1 (ERV9) | 2.97 |
| 121183 | AA400138 | Hs. 97703 | ESTs | 2.97 |
| 119241 | T12559 | Hs. 221382 | ESTs | 2.96 |
| 115067 | AA253458 | Hs. 91299 | postmeiotic segregation increased 2-like | 2.96 |
| 126196 | AA084394 | | zn05g10.s1 Stratagene hNT neuron (#93723 | 2.96 |
| 111642 | R16153 | Hs. 128740 | ESTs; Highly similar to DNb-5 [H. sapiens | 2.95 |
| 100898 | HG4638-HT5050 | | Spliceosomal Protein Sap 49 | 2.95 |
| 129370 | AA287879 | Hs. 110796 | ESTs; Moderately similar to GTP-binding | 2.94 |
| 128915 | C02386 | Hs. 107139 | ESTs | 2.94 |
| 101868 | M96233 | Hs. 82891 | glutathione S-transferase M4 | 2.94 |
| 124394 | N29724 | | gamma2-adaptin | 2.93 |
| 103559 | Z19585 | Hs. 75774 | thrombospondin 4 | 2.93 |
| 107882 | AA025630 | Hs. 17801 | ESTs; Moderately similar to serine/proli | 2.93 |
| 134919 | T99639 | Hs. 91142 | KH-type splicing regulatory protein (FUS | 2.92 |
| 110293 | H30258 | Hs. 37165 | collagen; type IX; alpha 2 | 2.92 |
| 132433 | AA082546 | Hs. 48516 | ESTs | 2.92 |
| 127347 | AA428350 | | ESTs | 2.92 |
| 121976 | AA429807 | Hs. 98632 | ESTs | 2.91 |
| 133025 | AA135492 | Hs. 6318 | ESTs; Highly similar to peroxisomal shor | 2.91 |
| 133413 | S72043 | Hs. 73133 | metallothionein 3 (growth inhibitory fac | 2.91 |
| 111694 | R22035 | Hs. 23331 | ESTs | 2.91 |
| 128369 | F12681 | Hs. 205300 | ESTs | 2.9 |
| 102464 | U49260 | Hs. 3828 | mevalonate (diphospho) decarboxylase | 2.9 |
| 135358 | C21431 | Hs. 99486 | ESTs; Weakly similar to aralar1 [H.sapie | 2.9 |
| 108661 | AA113287 | Hs. 65905 | ESTs; Weakly similar to PTB-ASSOCIATED S | 2.9 |
| 102185 | U20230 | | Human guanyl cyclase C gene, partial cds | 2.89 |
| 122071 | AA431787 | Hs. 98762 | EST | 2.89 |
| 102040 | U06088 | Hs. 159479 | galactosamine (N-acetyl)-6-sulfate sulfa | 2.89 |
| 115689 | AA410645 | Hs. 199014 | ESTs | 2.88 |
| 135110 | T15817 | Hs. 193788 | nitric oxide synthase 2A (inducible; hep | 2.88 |
| 118729 | N73717 | Hs. 161526 | EST | 2.88 |
| 129518 | AA369807 | Hs. 112238 | ESTs | 2.88 |
| 125788 | R74309 | Hs. 44499 | small EDRK-rich factor 2 | 2.87 |
| 128650 | U57971 | Hs. 103124 | ATPase; Ca++ transporting; plasma membra | 2.87 |
| 125936 | H30751 | Hs. 182859 | lifeguard | 2.87 |
| 100779 | HG3731-HT4001 | | Immunoglobulin Heavy Chain, Vdjrc Regions (Gb: L23566) | 2.87 |
| 104451 | M13299 | Hs. 102119 | blue cone pigment | 2.86 |
| 133539 | M21574 | Hs. 74615 | platelet-derived growth factor receptor; | 2.86 |
| 119506 | W37833 | Hs. 55563 | ESTs | 2.86 |
| 126568 | AA190515 | | zp85d12.r1 Stratagene HeLa cell s3 93721 | 2.86 |
| 134184 | X53742 | Hs. 79732 | fibulin 1 | 2.86 |
| 127633 | AI339609 | Hs. 152733 | potassium voltage-gated channel; Isk-rel | 2.86 |
| 128716 | AA045978 | Hs. 173611 | NADH dehydrogenase (ubiquinone) Fe-S pro | 2.86 |
| 107135 | AA620782 | Hs. 23247 | ESTs | 2.85 |
| 117748 | N47317 | Hs. 141858 | ESTs | 2.85 |
| 124030 | F04143 | Hs. 151032 | Homo sapiens clone 23856 unknown mRNA; p | 2.85 |
| 135120 | AA449841 | Hs. 108300 | NOT3 (negative regulator of transcriptio | 2.84 |
| 102156 | U17977 | | HSU17977 Humn fibroblast cDNA H sapiens | 2.84 |
| 129418 | AA401401 | Hs. 11127 | PET112 (yeast homolog)-like | 2.84 |
| 103222 | X74795 | Hs. 77171 | minichromosome maintenance deficient (S. | 2.84 |
| 125145 | W38001 | | Accession not listed in Genbank | 2.83 |
| 100560 | HG2228-HT2305 | | Crystallin, Beta B | 2.83 |
| 105370 | AA238476 | Hs. 22791 | ESTs; Weakly similar to transmembrane pr | 2.83 |
| 127036 | AI468598 | | ESTs | 2.83 |
| 128788 | AA029073 | Hs. 105685 | ESTs | 2.83 |
| 119523 | W38041 | | Accession not listed in Genbank | 2.82 |
| 126436 | N31224 | Hs. 211579 | melanoma adhesion molecule | 2.82 |
| 126559 | R15866 | Hs. 170263 | tumor protein 53-binding protein; 1 | 2.82 |
| 118183 | N59287 | Hs. 48361 | EST | 2.82 |
| 101298 | L40387 | Hs. 118633 | 2′-5′oligoadenylate synthetase-like | 2.81 |
| 131830 | U33054 | Hs. 32959 | G protein-coupled receptor kinase 2 (Dro | 2.81 |
| 124173 | H41281 | Hs. 107619 | ESTs | 2.81 |
| 102295 | U32581 | | Homo sapiens KIAA0421 mRNA; partial cds | 2.81 |
| 129719 | N66396 | Hs. 167766 | ESTs; Moderately similar to Pro-a2(XI) [ | 2.81 |
| 126573 | AA482023 | Hs. 155218 | E1B-55 kDa-associated protein 5 | 2.81 |
| 125477 | AI270093 | Hs. 234642 | aquaporin 3 | 2.81 |
| 106492 | AA451896 | Hs. 7922 | ESTs; Weakly similar to contains similar | 2.8 |
| | | | p19; an RNA polymerase II elongation fa | |
| 132881 | T86118 | Hs. 58875 | ESTs | 2.8 |
| 114733 | AA133778 | Hs. 95734 | ESTs | 2.79 |
| 104618 | AA001611 | Hs. 186494 | ESTs | 2.79 |
| 134137 | F10045 | Hs. 79347 | KIAA0211 gene product | 2.79 |
| 133212 | U82979 | Hs. 67846 | leukocyte Ig-like receptor; subfamily B | 2.78 |
| 100882 | HG4460-HT4729 | | Immunoglobulin Heavy Chain, Vdjc Regions (Gb: L23564) | 2.78 |
| 104756 | AA024622 | Hs. 15813 | solute carrier family 22 (organic cation | 2.78 |
| 129861 | N69507 | Hs. 129849 | DKFZP564M182 protein | 2.78 |
| 120824 | AA347548 | Hs. 96876 | ESTs | 2.78 |
| 100684 | HG3107-HT3283 | | Plasma Membrane Calcium Pump Hpmca2a | 2.78 |
| 121789 | AA423970 | Hs. 178111 | ESTs | 2.78 |
| 101847 | M59941 | Hs. 118200 | colony stimulating factor 2 receptor; be | 2.78 |
| 113722 | T97957 | Hs. 202948 | ESTs; Weakly similar to alternatively sp | 2.77 |
| 115107 | AA256371 | Hs. 186645 | ESTs | 2.77 |
| 111464 | R05518 | Hs. 19521 | ESTs | 2.77 |
| 108446 | AA079120 | | zm95e1.s1 Stratagene colon HT29 (#937221 | 2.77 |
| 123921 | AA621329 | Hs. 250671 | Hu DNA seq frm clone 1163J1 on chr 22q13 | 2.77 |
| | | | prot (similar to mouse Celsr1; rat MEGF | |
| 134445 | M59488 | Hs. 83384 | S100 calcium-binding protein; beta (neur | 2.76 |
| 114132 | Z38688 | Hs. 24192 | ESTs | 2.76 |
| 120500 | AA256430 | Hs. 132525 | ESTs | 2.76 |
| 101860 | M95610 | Hs. 37165 | collagen; type IX; alpha 2 | 2.76 |
| 134430 | H52105 | Hs. 8309 | KIAA0747 protein | 2.76 |
| 124152 | H27216 | Hs. 107635 | ESTs | 2.76 |
| 132268 | AA058833 | Hs. 23445 | ESTs; Weakly smlr to similar to M. muscu | 2.76 |
| 116257 | AA481493 | Hs. 88537 | ESTs | 2.76 |
| 102438 | U46570 | Hs. 7733 | tetratricopeptide repeat domain 1 | 2.75 |
| 122393 | AA446334 | Hs. 99064 | ESTs | 2.75 |
| 107653 | AA010210 | Hs. 47041 | ESTs | 2.75 |
| 123674 | AA609473 | Hs. 105187 | ESTs; Moderately similar to kinesin like | 2.75 |
| 129858 | T66906 | Hs. 12970 | ESTs | 2.75 |
| 130117 | U06641 | Hs. 150207 | UDP glycosyltransferase 2 family; polype | 2.75 |
| 133464 | M13982 | Hs. 73917 | interleukin 4 | 2.75 |
| 127039 | AA233366 | Hs. 256491 | ESTs | 2.74 |
| 128318 | AA418202 | Hs. 13810 | ESTs | 2.74 |
| 123363 | AA504818 | Hs. 171279 | ESTs | 2.74 |
| 127654 | AA649249 | Hs. 75640 | natriuretic peptide precursor A | 2.74 |
| 132067 | L20860 | Hs. 178382 | glycoprotein Ib (platelet); beta polypep | 2.74 |
| 125664 | AA948418 | Hs. 25744 | ESTs; Weakly similar to Ydr412wp [S.cere | 2.73 |
| 132354 | L05187 | Hs. 211913 | small proline-rich protein 1A | 2.73 |
| 101568 | M33764 | Hs. 75212 | ornithine decarboxylase 1 | 2.73 |
| 101438 | M20777 | Hs. 159263 | Homo sapiens; alpha-2 (VI) collagen | 2.73 |
| 116233 | AA479082 | Hs. 61142 | ESTs | 2.73 |
| 122194 | AA435882 | Hs. 97531 | ESTs | 2.72 |
| 113995 | W88466 | Hs. 22010 | ESTs | 2.72 |
| 124251 | H68286 | Hs. 107924 | ESTs | 2.71 |
| 120583 | AA281304 | Hs. 78614 | complement component 1; q subcomponent b | 2.71 |
| 134958 | U72507 | Hs. 234216 | Human 40871 mRNA partial sequence | 2.71 |
| 124280 | H85835 | Hs. 100058 | dihydropyrimidinase-like 4 | 2.71 |
| 130113 | M64673 | Hs. 1499 | heat shock transcription factor 1 | 2.71 |
| 106588 | AA456612 | Hs. 25682 | ESTs; Weakly smlr to PHOSPHATIDYLETHANOL | 2.71 |
| 132023 | F01927 | Hs. 3743 | ESTs; Weakly similar to proline-rich pro | 2.7 |
| 112284 | R53558 | Hs. 26052 | ESTs | 2.7 |
| 107897 | AA026240 | Hs. 61387 | ESTs | 2.7 |
| 122610 | AA453598 | Hs. 99336 | ESTs | 2.7 |
| 119070 | R27788 | Hs. 52302 | ESTs | 2.7 |
| 103491 | Y08836 | | Homo sapiens mRNA for HRX-like protein | 2.7 |
| 108225 | AA058843 | Hs. 161620 | EST | 2.7 |
| 105829 | AA398290 | Hs. 21965 | ESTs | 2.69 |
| 127749 | AI251757 | Hs. 145234 | ESTs | 2.69 |
| 128428 | AI185718 | Hs. 143900 | ESTs | 2.69 |
| 108409 | AA075578 | | zm88h3.s1 Stratagene ovarian cancer (#93 | 2.69 |
| 114739 | AA134923 | Hs. 103833 | ESTs; Weakly similar to predicted using | 2.68 |
| 128821 | D87002 | Hs. 135 | multiple UniGene matches | 2.68 |
| 107412 | W26105 | Hs. 8961 | ESTs | 2.68 |
| 117012 | H85893 | Hs. 194387 | ESTs; Weakly similar to !!!! ALU SUBFAMI | 2.68 |
| 135262 | AA416551 | Hs. 9732 | ESTs | 2.68 |
| 105367 | AA236397 | Hs. 20304 | ESTs | 2.68 |
| 134771 | L13939 | Hs. 89576 | adaptor-related protein complex 1; beta | 2.68 |
| 105036 | AA128617 | Hs. 25549 | ESTs | 2.68 |
| 125093 | T92930 | Hs. 186750 | ESTs | 2.68 |
| 119340 | T61899 | Hs. 90677 | ESTs; Highly similar to CGI-82 protein [ | 2.67 |
| 132603 | H62900 | Hs. 53066 | hsp70-interacting protein | 2.67 |
| 113733 | T98386 | Hs. 184548 | ESTs | 2.67 |
| 123564 | AA608902 | Hs. 112612 | ESTs | 2.66 |
| 116059 | AA454165 | Hs. 53455 | ESTs | 2.66 |
| 125803 | R79373 | Hs. 29852 | ESTs | 2.66 |
| 123012 | AA479962 | Hs. 139636 | EST | 2.66 |
| 106080 | AA418046 | Hs. 35124 | ESTs | 2.66 |
| 128809 | T59668 | Hs. 102267 | lysyl oxidase | 2.66 |
| 104354 | H08988 | Hs. 113759 | ESTs | 2.66 |
| 107068 | AA609028 | Hs. 8032 | ESTs | 2.65 |
| 101418 | M17754 | Hs. 1276 | BN51 (BHK21) temperature sensitivity com | 2.65 |
| 135157 | AA460138 | Hs. 95582 | SRY (sex-determining region Y)-box 20 | 2.65 |
| 123312 | AA496258 | Hs. 99601 | ESTs | 2.65 |
| 130034 | C00350 | Hs. 14454 | chromosome 2 open reading frame 1 | 2.65 |
| 103897 | AA248870 | Hs. 55058 | ESTs | 2.65 |
| 117771 | N47961 | Hs. 46794 | ESTs | 2.65 |
| 109980 | H09529 | Hs. 98693 | DKFZP586J0917 protein | 2.64 |
| 121966 | AA429653 | Hs. 98616 | EST | 2.64 |
| 114233 | Z39652 | Hs. 27457 | ESTs | 2.64 |
| 129594 | R70379 | Hs. 115396 | Human germline IgD chain gene; C-region; | 2.63 |
| 102319 | U34587 | Hs. 66578 | corticotropin releasing hormone receptor | 2.63 |
| 111700 | R22212 | Hs. 23361 | ESTs | 2.63 |
| 127365 | AA001628 | Hs. 74335 | heat shock 90 kD protein 1; beta | 2.63 |
| 104205 | AA496240 | Hs. 17270 | DKFZP434C211 protein | 2.63 |
| 124559 | N66223 | Hs. 135928 | ESTs; Weakly similar to !!!! ALU SUBFAMI | 2.63 |
| 106351 | AA442772 | Hs. 191987 | ESTs; Weakly similar to !!!! ALU SUBFAMI | 2.63 |
| 121903 | AA427605 | Hs. 258742 | myosin-binding protein C; cardiac | 2.62 |
| 116442 | AA620310 | Hs. 184343 | ESTs; Weakly similar to KIAA0585 protein | 2.62 |
| 127041 | F06090 | | HSC0WG031 normalized infant brain cDNA H | 2.62 |
| 132860 | U93049 | Hs. 58435 | FYN-binding protein (FYB-120/130) | 2.62 |
| 131591 | L22454 | Hs. 180069 | nuclear respiratory factor 1 | 2.61 |
| 118118 | N56901 | Hs. 47995 | ESTs | 2.61 |
| 134809 | X52611 | Hs. 18387 | transcription factor AP-2 alpha (activat | 2.61 |
| 117706 | N45091 | Hs. 46472 | ESTs | 2.61 |
| 127488 | AA312179 | Hs. 178617 | ESTs; Weakly similar to CGI-82 protein [ | 2.61 |
| 114891 | AA235984 | Hs. 87469 | ESTs | 2.6 |
| 116426 | AA609668 | Hs. 71657 | ESTs | 2.6 |
| 132589 | AA432197 | Hs. 5260 | ESTs; Weakly similar to CGI-08 protein [ | 2.6 |
| 128410 | AA452788 | | zx39g11.r1 Soares_total_fetus_Nb2HF8_9w | 2.6 |
| 106081 | AA418394 | Hs. 25354 | ESTs | 2.6 |
| 129919 | R02003 | Hs. 191208 | ESTs; Weakly similar to weak similarity | 2.59 |
| 124672 | R00307 | Hs. 188504 | ESTs | 2.59 |
| 122758 | AA459013 | Hs. 99742 | X-ray repair complementing defective rep | 2.59 |
| 125656 | AA040118 | Hs. 78687 | neutral sphingomyelinase (N-SMase) activ | 2.59 |
| 130052 | J00220 | Hs. 145288 | Human Ig active epsilon1 5′ UT; V-D-J re | 2.59 |
| 134878 | U28055 | Hs. 250826 | macrophage stimulating; pseudogene 9 | 2.59 |
| 131908 | L05624 | Hs. 3446 | mitogen-activated protein kinase kinase | 2.59 |
| 126470 | AA843339 | Hs. 193168 | ESTs; Weakly similar to CGI-52 protein [ | 2.59 |
| 132353 | M31651 | Hs. 46319 | sex hormone-binding globulin | 2.58 |
| 119588 | W44559 | Hs. 142525 | ESTs | 2.58 |
| 131757 | D17532 | Hs. 316 | DEAD/H (Asp-Glu-Ala-Asp/His) box polypep | 2.58 |
| 118114 | N56875 | Hs. 143212 | cystatin F (leukocystatin) | 2.58 |
| 128200 | AI279952 | Hs. 158037 | ESTs; Weakly similar to transcription re | 2.58 |
| 131208 | C14586 | Hs. 24220 | Homo sapiens mRNA; cDNA DKFZp566M051 (fr | 2.58 |
| 124721 | R11131 | Hs. 154966 | ESTs | 2.57 |
| 108706 | AA121820 | | Homo sapiens mRNA for KIAA0842 protein; | 2.57 |
| 118831 | N79592 | Hs. 50838 | ESTs | 2.57 |
| 115708 | AA412212 | Hs. 44033 | ESTs | 2.57 |
| 107233 | D59322 | Hs. 22595 | ESTs | 2.57 |
| 129559 | AA234945 | Hs. 11360 | ESTs | 2.57 |
| 126953 | AA743849 | Hs. 127286 | ESTs | 2.56 |
| 108165 | AA055221 | Hs. 63168 | ESTs | 2.56 |
| 104069 | AA401547 | Hs. 172694 | ESTs | 2.56 |
| 112146 | R46512 | Hs. 25374 | ESTs | 2.56 |
| 108364 | AA074891 | Hs. 124917 | ESTs; Highly similar to KIAA0838 protein | 2.56 |
| 131779 | R49047 | Hs. 179779 | ribosomal protein L37 | 2.56 |
| 111829 | R36070 | Hs. 25079 | EST | 2.55 |
| 103424 | X97267 | Hs. 155975 | protein tyrosine phosphatase; receptor t | 2.55 |
| 100133 | D13118 | Hs. 80986 | ATP synthase; H+ transporting; mitochond | 2.55 |
| 130208 | AA620556 | Hs. 15250 | peroxisomal D3;D2-enoyl-CoA isomerase | 2.55 |
| 124649 | N92593 | Hs. 102907 | ESTs | 2.55 |
| 106511 | AA452865 | Hs. 206713 | UDP-Gal: betaGlcNAc beta 1;4-galactosylt | 2.55 |
| 128467 | AA176446 | Hs. 180428 | ESTs; Weakly similar to hypothetical 43. | 2.55 |
| 113524 | T90072 | Hs. 15060 | ESTs | 2.55 |
| 107821 | AA020991 | Hs. 172856 | ESTs | 2.55 |
| 111900 | R39044 | Hs. 25318 | Homo sapiens clone 25194 mRNA sequence | 2.54 |
| 109908 | H05255 | Hs. 203237 | EST | 2.54 |
| 132069 | D87454 | Hs. 192966 | KIAA0265 protein | 2.54 |
| 130660 | T95262 | Hs. 17538 | ESTs | 2.54 |
| 112983 | T23443 | Hs. 7111 | ESTs | 2.54 |
| 128279 | H08885 | | yl88b08.r1 Soares infant brain 1NIB Homo | 2.54 |
| 106415 | AA447994 | Hs. 29188 | ESTs | 2.53 |
| 116741 | H03268 | Hs. 181746 | EST | 2.53 |
| 103148 | X66362 | Hs. 2994 | PCTAIRE protein kinase 3 | 2.53 |
| 132336 | AA342422 | Hs. 45073 | ESTs | 2.53 |
| 129484 | R92488 | Hs. 111989 | ESTs | 2.53 |
| 110169 | H19696 | Hs. 31612 | ESTs; Moderately similar to CAGH4 [H.sap | 2.53 |
| 116880 | H68380 | Hs. 144174 | EST | 2.53 |
| 133511 | X04106 | Hs. 74451 | calpain; small polypeptide | 2.53 |
| 126037 | M85772 | Hs. 6066 | KIAA1112 protein | 2.53 |
| 132678 | AA599876 | Hs. 5486 | ESTs | 2.53 |
| 128751 | AA442274 | Hs. 183176 | ESTs | 2.52 |
| 133664 | X86693 | Hs. 75445 | hevin | 2.52 |
| 126977 | AA309665 | | EST180547 Jurkat T-cells V Homo sapiens | 2.52 |
| 120697 | AA291522 | Hs. 97250 | EST | 2.52 |
| 128571 | AA416619 | Hs. 101661 | ESTs | 2.52 |
| 104422 | H86858 | Hs. 132909 | ESTs | 2.52 |
| 122372 | AA446008 | Hs. 99044 | EST | 2.52 |
| 112154 | R46769 | Hs. 25388 | ESTs | 2.52 |
| 126900 | R16034 | Hs. 12701 | ESTs; Highly similar to plasmolipin [H.s | 2.51 |
| 115000 | AA251342 | Hs. 144584 | ESTs | 2.51 |
| 110632 | H72344 | Hs. 171635 | ESTs | 2.51 |
| 129154 | N23673 | Hs. 108969 | mannosidase; alpha; class 2B; member 1 | 2.51 |
| 107440 | W28069 | Hs. 251993 | ESTs; Weakly similar to similar to zinc | 2.51 |
| 105694 | AA287109 | Hs. 37883 | ESTs | 2.51 |
| 106249 | AA430388 | Hs. 13144 | ESTs; Weakly similar to ORF YGR038w [S.c | 2.51 |
| 134462 | U11037 | Hs. 83620 | sel-1 (suppressor of lin-12; C.elegans)- | 2.51 |
| 101800 | M85276 | Hs. 105806 | granulysin | 2.51 |
| 119884 | W81606 | Hs. 58662 | Homo sapiens mRNA; cDNA DKFZp564G212 (fr | 2.51 |
| 110289 | H29829 | Hs. 31524 | ESTs | 2.51 |
| 125506 | H54273 | Hs. 154073 | UDP-galactose transporter related | 2.51 |
| 102954 | X15393 | Hs. 2813 | motilin | 2.51 |
| 127851 | AI469331 | Hs. 130497 | ESTs; Weakly similar to CHLORIDE CONDUCT | 2.5 |
| 126179 | AI191445 | Hs. 143855 | ESTs; Highly similar to IROQUOIS-CLASS H | 2.5 |
| 129443 | W69967 | Hs. 111497 | ESTs; Moderately similar to neuronal pro | 2.5 |
| 104480 | N41486 | Hs. 99654 | protein-O-mannosyltransferase 1 | 2.5 |
| 115580 | AA398695 | Hs. 144339 | Hu DNA seq frm clone 495O10 on chr 6q26- | 2.5 |
| | | | Prot L37A) pseudogene; last exon of gene for a novel prot smlr | |
| | | | to worm E04F6.2; ESTs; STSs and GSSs | |
| 119595 | W45031 | Hs. 55878 | EST | 2.5 |
| 103336 | X85785 | Hs. 183 | Duffy blood group | 2.5 |
| 102792 | U87964 | Hs. 227576 | GTP binding protein 1 | 2.49 |
| 129643 | L27584 | Hs. 250712 | calcium channel; voltage-dependent; beta | 2.49 |
| 134503 | U34880 | Hs. 84183 | diptheria toxin resistance protein reqrd | 2.49 |
| 117245 | N20989 | Hs. 42927 | ESTs | 2.49 |
| 126888 | H78745 | Hs. 1063 | small nuclear ribonucleoprotein polypept | 2.49 |
| 135313 | D63484 | Hs. 98508 | KIAA0150 protein | 2.49 |
| 121186 | AA400156 | Hs. 183294 | ESTs | 2.49 |
| 130651 | X04445 | Hs. 1734 | inhibin; alpha | 2.49 |
| 134218 | AA227480 | Hs. 80205 | pim-2 oncogene | 2.49 |
| 104008 | AA334630 | | EST38874 Embryo, 9 week Homo sapiens cDN | 2.49 |
| 129705 | X78706 | Hs. 12068 | camitine acetyltransferase | 2.49 |
| 127900 | AI143912 | Hs. 121824 | ESTs | 2.49 |
| 104609 | R96417 | Hs. 107795 | ESTs | 2.48 |
| 131628 | U47292 | Hs. 2979 | trefoil factor 2 (spasmolytic protein 1) | 2.48 |
| 132184 | U51003 | Hs. 419 | distal-less homeo box 2 | 2.48 |
| 130450 | U70735 | Hs. 15591 | COP9 subunit 6 (MOV34 homolog; 34 kD) | 2.48 |
| 101679 | M62628 | Hs. 163271 | Human alpha-1 Ig germline C-region membr | 2.48 |
| 120858 | AA350147 | Hs. 96940 | EST | 2.48 |
| 101012 | J04444 | Hs. 697 | cytochrome c-1 | 2.48 |
| 110453 | H52133 | Hs. 33026 | ESTs; Weakly similar to similar to Enter | 2.48 |
| 133771 | M68891 | Hs. 760 | GATA-binding protein 2 | 2.48 |
| 102944 | X14445 | Hs. 37092 | fibroblast grwth fctr 3 (murine mammary | 2.48 |
| 113269 | T65159 | Hs. 85044 | ESTs | 2.48 |
| 107069 | AA609045 | Hs. 11759 | ESTs; Weakly similar to !!!! ALU CLASS B | 2.48 |
| 100476 | HG1019-HT1019 | | Serine Kinase Psk-H1 | 2.47 |
| 106457 | AA449718 | Hs. 27801 | zinc finger protein 278 | 2.47 |
| 105718 | AA291629 | Hs. 74335 | heat shock 90 kD protein 1; beta | 2.47 |
| 104925 | AA058683 | Hs. 5548 | Homo sapiens clone 23765 mRNA sequence | 2.47 |
| 109913 | H05527 | Hs. 31588 | ESTs | 2.47 |
| 103412 | X96698 | Hs. 42957 | methyltransferase-like 1 | 2.47 |
| 102326 | U35246 | Hs. 226025 | vacuolar protein sorting 45A (yeast homo | 2.47 |
| 116813 | H49911 | Hs. 93102 | ESTs | 2.47 |
| 123690 | AA609566 | Hs. 112723 | EST | 2.47 |
| 124714 | R09486 | Hs. 193118 | ESTs | 2.47 |
| 126154 | AI004105 | Hs. 14232 | ESTs; Moderately similar to KIAA0563 pro | 2.47 |
| 118880 | N90168 | Hs. 54593 | EST | 2.47 |
| 122274 | AA437094 | Hs. 184456 | ESTs; Weakly similar to !!!! ALU SUBFAMI | 2.46 |
| 129600 | N78980 | Hs. 11567 | ESTs; Moderately similar to unknown [H.s | 2.46 |
| 121356 | AA405437 | Hs. 93581 | Homo sapiens mRNA; cDNA DKFZp586E171 (fr | 2.46 |
| 109560 | F01778 | Hs. 8154 | ESTs | 2.46 |
| 123342 | AA504336 | Hs. 31659 | thyroid hormone receptor-associated prot | 2.46 |
| 128032 | AI150084 | Hs. 126678 | ESTs | 2.46 |
| 129101 | H90310 | Hs. 108665 | ESTs; Weakly similar to CELL-CYCLE NUCLE | 2.46 |
| 131185 | M25753 | Hs. 23960 | cyclin B1 | 2.46 |
| 121451 | AA411008 | Hs. 98085 | EST | 2.46 |
| 104328 | D81932 | | HUM424C5B Hu fetal brain (TFujiwara) H.s | 2.46 |
| 126543 | AA723810 | Hs. 69517 | ESTs; Highly similar to differentially e | 2.45 |
| 123600 | AA609106 | Hs. 112644 | ESTs | 2.45 |
| 131020 | AA411756 | Hs. 20594 | ESTs; Weakly similar to misato [D.melano | 2.45 |
| 134191 | W28902 | Hs. 7979 | KIAA0736 gene product | 2.45 |
| 130446 | X79510 | Hs. 155693 | protein tyrosine phosphatase; non-recept | 2.45 |
| 131613 | R88228 | Hs. 29595 | JM4 protein | 2.45 |
| 118864 | N89670 | Hs. 42148 | ESTs; Weakly similar to Su(P) [D.melanog | 2.45 |
| 104232 | AB002351 | Hs. 10587 | KIAA0353 protein | 2.45 |
| 122604 | AA453489 | Hs. 99333 | ESTs | 2.45 |
| 120626 | AA285064 | Hs. 104485 | EST | 2.45 |
| 116655 | F03866 | Hs. 68090 | ESTs | 2.44 |
| 116267 | AA485080 | Hs. 256539 | ESTs | 2.44 |
| 114944 | AA243172 | Hs. 87619 | TED protein | 2.44 |
| 127629 | AA293279 | Hs. 29173 | ESTs | 2.44 |
| 120350 | AA211300 | Hs. 104166 | ESTs | 2.44 |
| 103620 | Z47087 | Hs. 182643 | transcription elongation factor B (SIII) | 2.44 |
| 131420 | Z11737 | Hs. 2664 | flavin containing monooxygenase 4 | 2.44 |
| 131312 | AA399226 | Hs. 25527 | tight junction protein 3 (zona occludens | 2.43 |
| 122812 | AA461044 | Hs. 142980 | EST | 2.43 |
| 135100 | AA398926 | Hs. 251108 | Homo sapiens mRNA; chromosome 1 specific | 2.43 |
| 113464 | T86931 | Hs. 16295 | ESTs | 2.43 |
| 100045 | M11507 | | AFFX control: transferrin receptor | 2.43 |
| 128975 | AA092129 | Hs. 107538 | ESTs; Moderately similar to/prediction | 2.43 |
| 103688 | AA011479 | Hs. 154701 | ESTs | 2.43 |
| 127331 | F20186 | | HSPD05873 HM3 Homo sapiens cDNA clone 05 | 2.43 |
| 107337 | T97111 | Hs. 191235 | ESTs; Weakly similar to Ydr324cp [S.cere | 2.43 |
| 122171 | AA435750 | Hs. 98830 | EST | 2.43 |
| 107601 | AA004636 | Hs. 50223 | ESTs | 2.43 |
| 119800 | W73523 | Hs. 58314 | ESTs | 2.43 |
| 104886 | AA053348 | Hs. 144626 | growth differentiation factor 11 | 2.42 |
| 122899 | AA469960 | Hs. 178420 | ESTs; Highly similar to WASP interacting | 2.42 |
| 125933 | AI308037 | Hs. 84120 | ESTs; Weakly similar to nucleoporin p62 | 2.42 |
| 121664 | AA417291 | Hs. 97978 | ESTs | 2.42 |
| 125450 | AA377194 | Hs. 238909 | ESTs; Weakly similar to POLYPOSIS LOCUS | 2.42 |
| 114611 | AA081374 | Hs. 108110 | DKFZP547E2110 protein | 2.42 |
| 111595 | R11492 | Hs. 191225 | ESTs | 2.42 |
| 111671 | R19368 | Hs. 229084 | EST | 2.42 |
| 110687 | H93005 | Hs. 177311 | ESTs | 2.42 |
| 103019 | X53414 | Hs. 144567 | alanine-glyoxylate aminotransferase (oxa | 2.42 |
| 119076 | R36634 | Hs. 235534 | ESTs | 2.42 |
| 130589 | AA234308 | Hs. 16441 | DKFZP434H204 protein | 2.42 |
| 125975 | AA495891 | Hs. 152290 | ESTs; Highly similar to PACAP type-3/VIP | 2.42 |
| 106380 | AA446188 | Hs. 16614 | ESTs | 2.41 |
| 121965 | AA429652 | Hs. 104901 | EST | 2.41 |
| 121604 | AA416788 | Hs. 98259 | EST | 2.41 |
| 100885 | HG4490-HT4876 | | Proline-Rich Protein Prb4, Allele | 2.41 |
| 117807 | N48701 | Hs. 46523 | EST | 2.41 |
| 119840 | W79525 | Hs. 58586 | ESTs | 2.41 |
| 102458 | U48861 | Hs. 54397 | cholinergic receptor, nicotinic; beta po | 2.41 |
| 116152 | AA460920 | Hs. 215683 | ESTs; Moderately similar to !!!! ALU SUB | 2.41 |
| 126741 | AA522512 | Hs. 29759 | Homo sapiens mRNA; cDNA DKFZp586L2123 (f | 2.41 |
| 103381 | X92715 | Hs. 3057 | zinc finger protein 74 (Cos52) | 2.41 |
| 124837 | R55630 | Hs. 233602 | KIAA0596 protein | 2.41 |
| 129322 | AA437153 | Hs. 110407 | ESTs; Weakly similar to coded for by C. | 2.4 |
| 129291 | AA281930 | Hs. 110099 | core-binding factor; runt domain; alpha | 2.4 |
| 124789 | R43803 | Hs. 78110 | ESTs; Weakly similar to F17A9.2 [C.elega | 2.4 |
| 133253 | Y00970 | Hs. 183088 | acrosin | 2.4 |
| 118990 | N94447 | Hs. 55047 | EST | 2.4 |
| 134897 | R71427 | Hs. 9081 | phenylalanyl-tRNA synthetase beta-subuni | 2.4 |
| 116572 | D45654 | Hs. 65582 | DKFZP586C1324 protein | 2.4 |
| 104294 | D14539 | Hs. 234774 | myeloid/lymphoid or mixed-lineage leukem | 2.4 |
| 118764 | N74440 | Hs. 205264 | ESTs | 2.4 |
| 117437 | N27645 | | yw5e3.s1 Weizmann Olfactory Epithelium H | 2.4 |
| | | | 3′ similar to contains L1.t3 L1 repetit | |
| 111651 | R16733 | Hs. 20499 | ESTs | 2.39 |
| 109583 | F02322 | Hs. 26135 | ESTs | 2.39 |
| 125969 | R94247 | Hs. 193879 | ESTs | 2.39 |
| 130647 | AA457216 | Hs. 214190 | interleukin enhancer binding factor 1 | 2.39 |
| 113708 | T97487 | Hs. 18065 | ESTs | 2.39 |
| 133469 | L03785 | Hs. 170482 | myosin; light polypeptide 5; regulatory | 2.39 |
| 118266 | N62837 | Hs. 48647 | immunoglobulin-like transcript 7 | 2.39 |
| 121656 | AA417248 | Hs. 98212 | ESTs | 2.39 |
| 126530 | AI422841 | Hs. 180086 | ESTs | 2.39 |
| 123708 | AA609648 | Hs. 207767 | EST | 2.39 |
| 107875 | AA025308 | Hs. 61182 | ESTs | 2.39 |
| 111711 | R22891 | Hs. 7093 | ESTs | 2.39 |
| 131405 | U79255 | Hs. 26468 | amyloid beta (A4) precursor protein-bind | 2.39 |
| 127454 | AA502957 | Hs. 153590 | ESTs | 2.39 |
| 132341 | AA448419 | Hs. 45209 | ESTs | 2.38 |
| 133673 | D87673 | Hs. 75486 | heat shock transcription factor 4 | 2.38 |
| 113213 | T58607 | | ya94a02.s1 Stratagene placenta (#937225) | 2.38 |
| 106230 | AA429356 | Hs. 12047 | ESTs | 2.38 |
| 116692 | F09261 | Hs. 66103 | ESTs | 2.38 |
| 126197 | AA172284 | Hs. 103657 | ESTs; Weakly similar to CH-TOG PROTEIN [ | 2.38 |
| 115966 | AA446866 | Hs. 71371 | ESTs | 2.38 |
| 132636 | U65785 | Hs. 5417 | oxygen regulated protein (150 kD) | 2.38 |
| 109965 | H09077 | Hs. 30895 | EST | 2.38 |
| 130203 | L14754 | Hs. 1521 | immunoglobulin mu binding protein 2 | 2.38 |
| 131332 | R50487 | Hs. 25717 | ESTs | 2.38 |
| 119105 | R42357 | Hs. 91453 | ESTs | 2.37 |
| 129253 | W69316 | Hs. 109778 | ESTs; Weakly similar to similar to beta- | 2.37 |
| 113602 | T92558 | Hs. 17036 | ESTs | 2.37 |
| 118102 | N55272 | Hs. 145798 | ESTs | 2.37 |
| 100734 | HG3432-HT3620 | | Fibroblast Growth Factor Receptor K-Sam, Alt. Splice 3, K-Sam lii | 2.37 |
| 111533 | R08548 | Hs. 251651 | EST | 2.37 |
| 130813 | U12259 | Hs. 198 | paired box gene 3 (Waardenburg syndrome | 2.37 |
| 119180 | R80413 | Hs. 92520 | ESTs | 2.37 |
| 109335 | AA211443 | Hs. 86492 | ESTs | 2.37 |
| 107386 | U97698 | Hs. 159593 | mucin 6; gastric | 2.36 |
| 122486 | AA448328 | Hs. 115527 | ESTs | 2.36 |
| 112997 | T23548 | Hs. 167467 | ESTs | 2.36 |
| 109674 | F09051 | Hs. 21837 | ESTs; Weakly similar to KIAA0927 protein | 2.36 |
| 128868 | AA423827 | Hs. 106730 | hypothetical protein | 2.36 |
| 127027 | R17261 | | yg12g07.r1 Soares infant brain 1NIB H.sa | 2.36 |
| 123099 | AA485931 | Hs. 79 | aminoacylase 1 | 2.36 |
| 115716 | AA416767 | Hs. 43498 | ESTs; Weakly similar to ORF YKL201c [S.c | 2.36 |
| 130830 | D86982 | Hs. 20060 | KIAA0229 protein | 2.36 |
| 109051 | AA159920 | Hs. 72322 | ESTs | 2.36 |
| 130181 | R39552 | Hs. 151608 | Homo sapiens clone 23622 mRNA sequence | 2.36 |
| 131114 | R46233 | Hs. 23107 | ESTs | 2.36 |
| 123589 | AA609047 | Hs. 188922 | ESTs | 2.36 |
| 130872 | U03891 | | phorbolin (similar to apolipoprotein B m | 2.36 |
| 131962 | H78550 | Hs. 2780 | jun D proto-oncogene | 2.36 |
| 130502 | M55067 | Hs. 1583 | neutrophil cytosolic factor 1 (47 kD; chr | 2.36 |
| 121785 | AA423883 | Hs. 142442 | ESTs | 2.35 |
| 125405 | T97171 | Hs. 121570 | ESTs | 2.35 |
| 103682 | AA000993 | | ESTs | 2.35 |
| 125649 | T77395 | Hs. 194816 | stomatin-like protein 1 | 2.35 |
| 115452 | AA285019 | Hs. 55263 | ESTs; Highly similar to mitochondrial di | 2.35 |
| 129338 | T56800 | Hs. 47274 | Homo sapiens mRNA; cDNA DKFZp564B176 (fr | 2.35 |
| 106105 | AA421268 | Hs. 149443 | putative tumor suppressor | 2.35 |
| 134770 | R72079 | Hs. 89575 | CD79B antigen (immunoglobulin-associated | 2.35 |
| 119422 | T99496 | Hs. 229598 | EST | 2.35 |
| 109869 | H02849 | Hs. 30345 | EST | 2.35 |
| 134314 | AA263032 | Hs. 81634 | ATP synthase; H+ transporting; mitochond | 2.35 |
| 114989 | AA251097 | Hs. 189119 | ESTs | 2.35 |
| 122619 | AA453755 | Hs. 191515 | ESTs | 2.35 |
| 133129 | AA428580 | Hs. 65551 | ESTs | 2.35 |
| 128465 | AA416762 | Hs. 100221 | nuclear receptor subfamily 1; group H; m | 2.35 |
| 115636 | AA402715 | Hs. 58389 | ESTs | 2.35 |
| 130836 | J05068 | Hs. 2012 | transcobalamin I (vitamin B12 binding pr | 2.34 |
| 132385 | Y10256 | Hs. 47007 | serine/threonine protein-kinase | 2.34 |
| 107776 | AA018820 | Hs. 221147 | ESTs | 2.34 |
| 109791 | F10669 | Hs. 13228 | DRE-antagonist modulator; calsenilin | 2.34 |
| 124409 | N33212 | Hs. 107197 | ESTs | 2.34 |
| 131068 | AA397916 | Hs. 22595 | ESTs | 2.34 |
| 121079 | AA398719 | Hs. 14169 | ESTs; Weakly similar to CREB-binding pro | 2.34 |
| 124662 | N94340 | Hs. 171835 | ESTs; Weakly smlr to PUT PRE-MRNA SPLICI | 2.34 |
| 133820 | M13686 | Hs. 177582 | surfactant; pulmonary-associated protein | 2.34 |
| 129424 | M55593 | Hs. 111301 | matrix metalloproteinase2 (gelatinase A | 2.34 |
| 109066 | AA161377 | Hs. 72404 | EST | 2.34 |
| 100339 | D63485 | Hs. 181359 | KIAA0151 gene product | 2.34 |
| 100809 | HG3991-HT4261 | | Cpg-Enriched Dna, Clone E18 | 2.34 |
| 120844 | AA349417 | Hs. 96917 | ESTs | 2.33 |
| 124927 | R96146 | Hs. 221459 | ESTs | 2.33 |
| 109779 | F10527 | Hs. 3353 | Homo sapiens clone 24940 mRNA sequence | 2.33 |
| 101171 | L16842 | Hs. 119251 | ubiquinol-cytochrome c reductase core pr | 2.33 |
| 110805 | N26904 | Hs. 24048 | ESTs; Weakly similar to FK506/rapamycin- | 2.33 |
| 125440 | AI090982 | Hs. 31895 | ESTs | 2.33 |
| 133159 | AC000061 | Hs. 663 | cystic fibrosis transmemb conductance re | 2.33 |
| 101829 | M91368 | Hs. 129763 | solute carrier family 8 (sodium/calcium | 2.33 |
| 126492 | AA778565 | Hs. 142505 | ESTs | 2.33 |
| 102774 | U83303 | Hs. 164021 | small inducible cytokine subfamily B (CX | 2.33 |
| 130480 | N50809 | Hs. 15760 | ESTs; Weakly similar to similar to Yeast | 2.33 |
| 126878 | AI424759 | Hs. 238928 | ESTs | 2.33 |
| 117338 | N23889 | Hs. 43466 | ESTs | 2.32 |
| 118662 | N70877 | Hs. 13055 | ESTs | 2.32 |
| 130354 | AA416685 | Hs. 155001 | UNC13 (C. elegans)-like | 2.32 |
| 106760 | AA477330 | Hs. 12293 | ESTs | 2.32 |
| 124294 | H90573 | Hs. 102298 | EST | 2.32 |
| 119428 | W02129 | Hs. 55242 | EST | 2.32 |
| 132629 | Z40942 | Hs. 5383 | ESTs | 2.32 |
| 127998 | AA854161 | Hs. 143585 | ESTs | 2.32 |
| 132728 | AA293334 | Hs. 5566 | ESTs; Highly similar to RAS-RELATED PROT | 2.32 |
| 120292 | AA189116 | Hs. 96168 | ESTs | 2.32 |
| 107598 | AA004528 | Hs. 169444 | ESTs | 2.32 |
| 128164 | AI478174 | Hs. 144846 | ESTs | 2.32 |
| 105753 | AA299789 | Hs. 15277 | ESTs | 2.31 |
| 131256 | AA262340 | Hs. 24907 | coronin; actin-binding protein: 2B | 2.31 |
| 110891 | N38863 | Hs. 234392 | platelet-activating factor acetylhydrola | 2.31 |
| 116767 | H13689 | Hs. 92530 | ESTs | 2.31 |
| 100545 | HG2147-HT2217 | | Mucin 3, Intestinal (Gb: M55405) | 2.31 |
| 125264 | W88995 | Hs. 167641 | ESTs; Weakly similar to C15H9.5 [C.elega | 2.31 |
| 118387 | N64579 | | yz51d11.s1 Morton Fetal Cochlea H.sapien | 2.31 |
| 104335 | D83847 | Hs. 183864 | elastase 3B | 2.31 |
| 107464 | W42944 | Hs. 171939 | ESTs | 2.31 |
| 112304 | R54798 | Hs. 26239 | ESTs | 2.31 |
| 134313 | AA136100 | Hs. 6673 | trinucleotide repeat containing 15 | 2.31 |
| 116322 | AA490900 | Hs. 58643 | ESTs; Highly similar to JAK3B [H. sapiens | 2.31 |
| 111275 | N70970 | Hs. 35006 | ESTs | 2.31 |
| 100109 | AJ000480 | Hs. 143513 | phosphoprotein regulated by mitogenic pa | 2.31 |
| 109338 | AA211717 | Hs. 86507 | ESTs | 2.31 |
| 134432 | AA053022 | Hs. 8312 | ESTs | 2.31 |
| 129649 | AD000092 | Hs. 182628 | Homo sapiens DNA from chr 19p13.2 cosmid | 2.31 |
| | | | EKLF; GCDH; CRTC; and RAD23A genes; gen | |
| 122623 | AA453990 | Hs. 99248 | ESTs | 2.31 |
| 112070 | R43976 | Hs. 236310 | EST | 2.31 |
| 127683 | AA668123 | Hs. 134170 | ESTs | 2.31 |
| 104920 | AA057620 | Hs. 30807 | ESTs; Highly similar to dJ186O1.1 [H.sap | 2.31 |
| 106064 | AA417373 | Hs. 15898 | ESTs | 2.31 |
| 106782 | AA478487 | | ESTs | 2.31 |
| 126709 | AA028159 | Hs. 47234 | ESTs | 2.3 |
| 105129 | AA158386 | Hs. 186476 | ESTs | 2.3 |
| 105719 | AA291644 | Hs. 36793 | ESTs | 2.3 |
| 121698 | AA418399 | Hs. 10351 | KIAA0308 protein | 2.3 |
| 119069 | R27619 | Hs. 231046 | EST | 2.3 |
| 130388 | U72515 | Hs. 189583 | putative protein similar to nessy (Droso | 2.3 |
| 103444 | X98801 | Hs. 74617 | dynactin 1 (p150; Glued (Drosophila) hom | 2.3 |
| 114604 | AA076128 | | zm18g4.s1 Stratagene pancreas (#93728) H | 2.3 |
| | | | 3′ similar to SW: RS1A_HUMAN P3927 4S RI | 2.3 |
| 103878 | AA227635 | Hs. 202588 | ESTs | 2.3 |
| 105828 | AA398276 | Hs. 11962 | ESTs | 2.3 |
| 119778 | W72920 | Hs. 58244 | ESTs | 2.3 |
| 120401 | AA234309 | Hs. 193011 | ESTs | 2.3 |
| 116290 | AA488691 | Hs. 57969 | phenylalanine-tRNA synthetase | 2.3 |
| 130479 | R44163 | Hs. 12457 | Homo sapiens clone 23770 mRNA sequence | 2.3 |
| 104253 | AF002672 | Hs. 152944 | loss of heterozygosity; 11; chromosomal | 2.29 |
| 132615 | H66367 | Hs. 53358 | ESTs; Weakly similar to !!!! ALU SUBFAMI | 2.29 |
| 121954 | AA429598 | Hs. 98587 | ESTs | 2.29 |
| 101336 | L49169 | Hs. 75678 | FBJ murine osteosarcoma viral oncogene h | 2.29 |
| 127247 | AA313802 | Hs. 6289 | growth factor receptor-bound protein 2 | 2.29 |
| 117300 | N22565 | Hs. 43212 | ESTs | 2.29 |
| 122229 | AA436198 | Hs. 103902 | ESTs | 2.29 |
| 125106 | T95766 | Hs. 189760 | ESTs | 2.29 |
| 128083 | R16100 | Hs. 166476 | ESTs | 2.29 |
| 131279 | AA089853 | Hs. 25197 | STIP1 homology and U-Box containing prot | 2.29 |
| 133838 | M97796 | Hs. 180919 | inhibitor of DNA binding 2; dominant neg | 2.29 |
| 111837 | R36447 | Hs. 24453 | ESTs | 2.29 |
| 111435 | R01620 | Hs. 19198 | ESTs | 2.29 |
| 123613 | AA609158 | Hs. 112656 | EST | 2.29 |
| 133560 | AA256365 | Hs. 7486 | protein expressed in thyroid | 2.29 |
| 122896 | AA469952 | Hs. 97899 | ESTs; Weakly similar to dal2; len: 343; C | 2.29 |
| 113378 | T80627 | Hs. 14757 | ESTs | 2.29 |
| 127174 | AA293204 | Hs. 139352 | ESTs | 2.29 |
| 120153 | Z39582 | Hs. 65777 | EST | 2.29 |
| 112741 | R93080 | Hs. 35035 | ESTs | 2.28 |
| 132152 | AA044784 | Hs. 4105 | Homo sapiens mRNA; cDNA DKFZp586A0618 (f | 2.28 |
| 109790 | F10665 | Hs. 25031 | ESTs | 2.28 |
| 113776 | W04657 | Hs. 24248 | ESTs | 2.28 |
| 102934 | X13451 | | Hu mRNA for lymphocyte lineage-rstrcted | 2.28 |
| 126188 | AA322034 | | EST24690 Cerebellum II Homo sapiens cDNA | 2.28 |
| 126363 | N94706 | | Human Chromosome 16 BAC clone CIT987SK-A | 2.28 |
| 101427 | M19508 | | Human myeloperoxidase gene, exons 1-4 | 2.28 |
| 132616 | AA386264 | Hs. 5337 | isocitrate dehydrogenase 2 (NADP+); mito | 2.28 |
| 105537 | AA258813 | Hs. 27160 | ESTs | 2.28 |
| 126527 | AA548559 | Hs. 103853 | ESTs | 2.28 |
| 115359 | AA281936 | Hs. 88914 | ESTs | 2.28 |
| 108474 | AA079667 | | zm93d1.s1 Stratagene ovarian cncr (#9372 | 2.28 |
| 120685 | AA291066 | Hs. 105099 | ESTs | 2.28 |
| 126171 | AA704771 | Hs. 191942 | ESTs | 2.28 |
| 112858 | T02963 | Hs. 4454 | ESTs | 2.28 |
| 121817 | AA424826 | Hs. 98475 | EST | 2.28 |
| 107895 | AA026150 | Hs. 61384 | ESTs | 2.28 |
| 131161 | Z38223 | Hs. 23735 | potassium voltage-gated channel; subfami | 2.28 |
| 135173 | M72885 | Hs. 95910 | Human G0S2 protein gene; complete cds | 2.27 |
| 103182 | X69819 | Hs. 99995 | intercellular adhesion molecule 3 | 2.27 |
| 113889 | W72720 | Hs. 194347 | ESTs | 2.27 |
| 128984 | AA319615 | Hs. 238030 | secretory carrier membrane protein 2 | 2.27 |
| 101531 | M29877 | Hs. 576 | fucosidase; alpha-L-1; tissue | 2.27 |
| 115916 | AA436889 | Hs. 91910 | ESTs | 2.27 |
| 129892 | H96850 | Hs. 89674 | dolichyl-diphosphooligosaccharide-protei | 2.27 |
| 103035 | X54871 | Hs. 77690 | RAB5B; member RAS oncogene family | 2.27 |
| 126479 | T78141 | | ESTs | 2.27 |
| 125778 | R71976 | Hs. 161791 | ESTs; Weakly similar to !!!! ALU SUBFAMI | 2.27 |
| 108132 | AA053586 | Hs. 63048 | ESTs | 2.27 |
| 111017 | N53965 | Hs. 256327 | ESTs | 2.27 |
| 127165 | AA359719 | Hs. 127121 | ESTs | 2.27 |
| 126446 | AI421309 | Hs. 118926 | DKFZP586K0919 protein | 2.26 |
| 107864 | AA025061 | Hs. 61246 | ESTs | 2.26 |
| 122277 | AA437133 | Hs. 98936 | ESTs | 2.26 |
| 115604 | AA400378 | Hs. 49391 | ESTs | 2.26 |
| 105061 | AA134824 | Hs. 4865 | ESTs | 2.26 |
| 118549 | N68163 | Hs. 49455 | EST | 2.26 |
| 110509 | H56493 | Hs. 61960 | ESTs; Moderately similar to HYPOTHETICAL | 2.26 |
| 114088 | Z38280 | Hs. 26971 | Human Chromosome 16 BAC clone CIT987SK-2 | 2.26 |
| 103225 | X74837 | Hs. 2750 | mannosidase; alpha; class 1A; member 1 | 2.26 |
| 125842 | AA746654 | Hs. 5181 | proliferation-associated 2G4; 38 kD | 2.26 |
| 104538 | R25069 | Hs. 175681 | ESTs | 2.26 |
| 130304 | U09368 | Hs. 154205 | zinc finger protein 140 (clone pHZ-39) | 2.26 |
| 120680 | AA290743 | Hs. 97242 | ESTs | 2.26 |
| 124062 | H00440 | Hs. 144524 | ESTs; Weakly similar to signal transduce | 2.26 |
| 103289 | X80915 | Hs. 1573 | growth differentiation factor 5 (cartila | 2.26 |
| 109286 | AA197273 | Hs. 191324 | ESTs | 2.26 |
| 128555 | U62739 | Hs. 101408 | branched chain aminotransferase 2; mitoc | 2.26 |
| 129439 | AA171694 | Hs. 111461 | ceruloplasmin (ferroxidase) | 2.26 |
| 109221 | AA192755 | Hs. 85840 | ESTs; Weakly similar to stac [H. sapiens] | 2.26 |
| 109906 | H05084 | Hs. 28077 | ESTs; Highly similar to GDP-mannose pyro | 2.26 |
| 130540 | U35234 | Hs. 159534 | protein tyrosine phosphatase; receptor t | 2.26 |
| 122870 | AA405158 | Hs. 192861 | Spi-B transcription factor (Spi-1/PU.1 r | 2.26 |
| 120219 | Z41124 | Hs. 66045 | EST | 2.26 |
| 128021 | AI001136 | Hs. 78223 | N-acylaminoacyl-peptide hydrolase | 2.26 |
| 121732 | AA421047 | Hs. 98330 | ESTs | 2.26 |
| 107817 | AA020781 | Hs. 60847 | ESTs | 2.25 |
| 101069 | L02648 | Hs. 84232 | transcobalamin II; macrocytic anemia | 2.25 |
| 103065 | X58399 | Hs. 81221 | Human L2-9 transcript of unrearranged im | 2.25 |
| 118019 | N52585 | Hs. 47517 | ESTs | 2.25 |
| 122220 | AA436011 | Hs. 98187 | ESTs | 2.25 |
| 109161 | AA179392 | Hs. 73601 | EST | 2.25 |
| 128699 | K03207 | Hs. 103972 | proline-rich protein BstNI subfamily 4 | 2.25 |
| 101914 | S71824 | Hs. 167988 | neural cell adhesion molecule 1 | 2.25 |
| 102697 | U74667 | Hs. 6364 | Tat interactive protein (60 kD) | 2.25 |
| 119939 | AA86753 | Hs. 82407 | ESTs | 2.25 |
| 127793 | AI298835 | Hs. 30445 | ESTs; Weakly similar to transcription re | 2.25 |
| 104450 | L77564 | Hs. 103978 | serine/threonine kinase 22B (spermiogene | 2.25 |
| 133096 | AA136042 | Hs. 131053 | ESTs | 2.25 |
| 115416 | AA283893 | Hs. 203866 | ESTs | 2.25 |
| 117058 | H90322 | Hs. 41387 | EST | 2.25 |
| 115598 | AA400129 | Hs. 65735 | ESTs | 2.25 |
| 121267 | AA401397 | Hs. 165296 | ESTs; Highly similar to kallikrein-like | 2.25 |
| 104778 | AA026397 | Hs. 11039 | Homo sapiens clone 24804 mRNA sequence | 2.25 |
| 110926 | N48252 | Hs. 135287 | ESTs | 2.24 |
| 102795 | U88667 | Hs. 198396 | ATP-binding cassette; sub-family A (ABC1 | 2.24 |
| 118643 | N70324 | Hs. 49840 | ESTs | 2.24 |
| 103304 | X82240 | Hs. 2484 | T-cell leukemia/lymphoma 1A | 2.24 |
| 134814 | Z48475 | Hs. 89771 | glucokinase (hexokinase 4) regulatory pr | 2.24 |
| 125912 | AA171719 | Hs. 5233 | eukaryotic translation initiation factor | 2.24 |
| 134365 | R32377 | Hs. 82240 | syntaxin 3A | 2.24 |
| 117224 | N20300 | Hs. 218707 | ESTs | 2.24 |
| 107169 | AA621601 | Hs. 184446 | ESTs; Weakly similar to small GTP-bindin | 2.24 |
| 133948 | M59916 | Hs. 77813 | sphingomyelin phosphodiesterase 1; acid | 2.24 |
| 101426 | M19483 | Hs. 25 | ATP synthase; H+ transporting; mitochond | 2.24 |
| 119922 | W86196 | Hs. 177384 | ESTs | 2.24 |
| 123361 | AA504810 | Hs. 139649 | EST | 2.24 |
| 123915 | AA621298 | Hs. 112967 | ESTs | 2.24 |
| 123540 | AA608792 | Hs. 112591 | EST | 2.24 |
| 124978 | T40560 | Hs. 221759 | ESTs | 2.24 |
| 102354 | U38268 | | Human cytochrome b pseudogene, partial c | 2.24 |
| 124198 | H53099 | Hs. 198271 | NADH dehydrogenase (ubiquinone) 1 alpha | 2.24 |
| 102160 | U18235 | Hs. 121561 | ATP-binding cassette; sub-family A (ABC1 | 2.24 |
| 107520 | X76091 | Hs. 100007 | regulatory factor X; 2 (influences HLA c | 2.24 |
| 131589 | U52100 | Hs. 29191 | epithelial membrane protein 2 | 2.24 |
| 126633 | AA206993 | Hs. 154145 | guanine nucl binding protein (G protein) | 2.23 |
| 130887 | AA258379 | Hs. 155986 | angiotensin receptor-like 2 | 2.23 |
| 119894 | W84670 | Hs. 58518 | EST | 2.23 |
| 124544 | N63837 | Hs. 40500 | similar to S. cerevisiae RER1 | 2.23 |
| 103104 | X61587 | Hs. 75082 | ras homolog gene family; member G (rho G | 2.23 |
| 110119 | H17306 | Hs. 177229 | ESTs | 2.23 |
| 131411 | AA464043 | Hs. 26506 | ESTs; Weakly similar to NY-REN-45 antige | 2.23 |
| 102346 | U37359 | Hs. 227297 | meiotic recombination (S. cerevisiae) 11 | 2.23 |
| 106003 | AA411167 | Hs. 8734 | ESTs; Moderately similar to !!!! ALU CLA | 2.23 |
| 122564 | AA452251 | Hs. 98669 | ESTs | 2.23 |
| 133688 | U42031 | Hs. 7557 | FK506-binding protein 5 | 2.23 |
| 132096 | AA131410 | Hs. 3964 | Homo sapiens clone 24877 mRNA sequence | 2.23 |
| 110038 | H11746 | Hs. 31097 | ESTs | 2.23 |
| 123788 | AA620293 | Hs. 112853 | ESTs | 2.23 |
| 135070 | X99350 | Hs. 93974 | forkhead box J1 | 2.23 |
| 104908 | AA055841 | Hs. 154396 | ESTs | 2.22 |
| 128674 | AA025001 | Hs. 169452 | ESTs | 2.22 |
| 100810 | HG3992-HT4262 | | Cpg-Enriched Dna, Clone E35 | 2.22 |
| 120065 | W93579 | Hs. 59478 | EST | 2.22 |
| 122775 | AA459692 | Hs. 112143 | ESTs | 2.22 |
| 125443 | H71482 | Hs. 177592 | tibosomal protein; large; P1 | 2.22 |
| 118617 | N69666 | Hs. 183413 | ESTs; Moderately similar to !!!! ALU SUB | 2.22 |
| 128001 | AI167814 | Hs. 166664 | ESTs | 2.22 |
| 128160 | AI279080 | Hs. 149971 | ESTs; Moderately similar to !!!! ALU CLA | 2.22 |
| 106608 | AA458644 | Hs. 27115 | ESTs | 2.22 |
| 103485 | Y08409 | Hs. 248415 | thyroid hormone responsive SPOT14 (rat) | 2.22 |
| 135008 | AA173423 | Hs. 92918 | ESTs; Weakly similar to R07G3.8 [C.elega | 2.22 |
| 110122 | H17333 | Hs. 159837 | EST | 2.22 |
| 128397 | AI393421 | Hs. 14032 | ESTs | 2.22 |
| 110231 | H24359 | Hs. 28733 | ESTs | 2.22 |
| 123188 | AA489092 | Hs. 177726 | ESTs | 2.22 |
| 131903 | AA481723 | Hs. 3436 | deleted in oral cancer (mouse; homolog) | 2.22 |
| 122649 | AA454616 | Hs. 90336 | ATPase; H+ transporting; lysosomal (vacu | 2.22 |
| 133090 | AA448228 | Hs. 6468 | ESTs | 2.22 |
| 108002 | AA037664 | Hs. 55067 | ESTs; Weakly similar to T07F12.1 gene pr | 2.22 |
| 133120 | X64559 | Hs. 65424 | tetranectin (plasminogen-binding protein | 2.21 |
| 114263 | Z40073 | Hs. 6045 | ESTs | 2.21 |
| 125518 | R20148 | Hs. 193851 | ESTs | 2.21 |
| 128613 | U78551 | Hs. 102482 | Homo sapiens gallbladder mucin MUC5B mRN | 2.21 |
| 102773 | U83192 | Hs. 23731 | discs; large (Drosophila) homolog 4 | 2.21 |
| 119526 | W38049 | | Accession not listed in Genbank | 2.21 |
| 126844 | AA299325 | | EST11903 Uterus tumor I Homo sapiens cDN | 2.21 |
| 105860 | AA399251 | Hs. 180933 | ESTs; Weakly similar to methyl-CpG bindi | 2.21 |
| 126957 | AA733145 | Hs. 194560 | ESTs | 2.21 |
| 108959 | AA150107 | Hs. 81810 | ESTs | 2.2 |
| 131663 | AA423926 | Hs. 30318 | ESTs | 2.2 |
| 127468 | H02941 | Hs. 8888 | ESTs | 2.2 |
| 104483 | N42776 | Hs. 146233 | EST | 2.2 |
| 123848 | AA620773 | Hs. 221996 | ESTs | 2.2 |
| 101623 | M55905 | Hs. 75342 | malic enzyme 2; NAD(+)-dependent; mitoch | 2.2 |
| 120872 | AA357993 | Hs. 96996 | ESTs | 2.2 |
| 135033 | AA173241 | Hs. 93454 | ESTs | 2.2 |
| 122286 | AA437259 | Hs. 104944 | EST | 2.2 |
| 114862 | AA235174 | Hs. 50250 | ESTs | 2.2 |
| 100255 | D38047 | Hs. 78466 | proteasome (prosome; macropain) 26S subu | 2.2 |
| 103063 | X58234 | Hs. 123178 | translocase of inner mitochondrial membr | 2.2 |
| 132777 | R56898 | Hs. 56663 | ESTs | 2.2 |
| 133082 | AA457129 | Hs. 6455 | RuvB (E. coli homolog)-like 2 | 2.2 |
| 127529 | AA558980 | Hs. 191750 | ESTs | 2.2 |
| 114602 | AA075642 | Hs. 103594 | deleted in malignant brain tumors 1 | 2.2 |
| 120722 | AA293435 | Hs. 97277 | ESTs | 2.2 |
| 102675 | U72512 | | Human B-cell receptor associated protein | 2.2 |
| 128551 | H09058 | Hs. 237323 | N-acetylglucosamine-phosphate mutase; DK | 2.2 |
| 112020 | R43001 | Hs. 22298 | EST | 2.2 |
| 123625 | AA609216 | Hs. 112666 | EST | 2.2 |
| 120315 | AA194266 | Hs. 178393 | ESTs | 2.2 |
| 122081 | AA431992 | Hs. 104920 | ESTs | 2.19 |
| 101798 | M85220 | | Accession not listed in Genbank | 2.19 |
| 111501 | R07444 | Hs. 163118 | ESTs | 2.19 |
| 132832 | D63482 | Hs. 57734 | KIAA0148 gene product | 2.19 |
| 100544 | HG2147-HT2217 | | Mucin 3, Intestinal (Gb: M55405) | 2.19 |
| 106835 | AA482077 | Hs. 33713 | ESTs; Weakly similar to hypothetical pro | 2.19 |
| 132934 | AA076145 | Hs. 61053 | ESTs | 2.19 |
| 108762 | AA127515 | Hs. 71787 | ESTs; Highly similar to 30S ribosomal pr | 2.19 |
| 120164 | Z39733 | Hs. 158159 | FAT tumor suppressor (Drosophila) homolo | 2.19 |
| 135395 | L08096 | Hs. 99899 | tumor necrosis factor (ligand) superfami | 2.19 |
| 101717 | M69013 | Hs. 1686 | guanine nucleotide binding protein (G pr | 2.19 |
| 121172 | AA400013 | Hs. 97750 | EST | 2.18 |
| 114861 | AA235123 | Hs. 40719 | ESTs | 2.18 |
| 120851 | AA349662 | Hs. 174248 | ESTs | 2.18 |
| 121083 | AA398736 | Hs. 97653 | EST | 2.18 |
| 107171 | AA621624 | Hs. 28088 | Homo sapiens clone 24515 mRNA sequence | 2.18 |
| 128754 | D31446 | Hs. 10488 | Breakpoint cluster region protein; uteri | 2.18 |
| 100149 | D13897 | Hs. 169249 | peptide YY | 2.18 |
| 132405 | AA323787 | Hs. 4770 | KIAA1068 protein | 2.18 |
| 114666 | AA112274 | | zm27g6.s1 Stratagene pancreas (#93728) H | 2.18 |
| | | | element; contains element LTR8 repetitiv | |
| 127008 | AA223879 | | zr10g05.r1 Stratagene NT2 neuronal precu | 2.18 |
| 110373 | H42896 | Hs. 29438 | ESTs | 2.18 |
| 119354 | T66942 | Hs. 100651 | golgi SNAP receptor complex member 2 | 2.18 |
| 130115 | M31627 | Hs. 149923 | X-box binding protein 1 | 2.18 |
| 130514 | AA161085 | Hs. 15871 | ESTs; Weakly similar to acid phosphatase | 2.18 |
| 128848 | H08077 | Hs. 217179 | ESTs; Weakly similar to T27A1.5 [C.elega | 2.18 |
| 110161 | H19312 | Hs. 28096 | ESTs | 2.18 |
| 132367 | X82224 | Hs. 46634 | cysteine conjugate-beta lyase; cytoplasm | 2.18 |
| 125882 | H45538 | Hs. 101448 | metastasis associated 1 | 2.17 |
| 113837 | W57698 | Hs. 8888 | ESTs | 2.17 |
| 106376 | AA444004 | Hs. 6084 | ESTs | 2.17 |
| 113755 | T99075 | Hs. 18570 | ESTs | 2.17 |
| 107525 | X91817 | Hs. 102866 | transketolase-like 1 | 2.17 |
| 119207 | R93186 | Hs. 84298 | CD74 antigen (invar polypept of maj hist | 2.17 |
| 131862 | AA236365 | | 3-phosphoglycerate dehydrogenase | 2.17 |
| 115514 | AA297739 | Hs. 55609 | ESTs; Weakly similar to ISOLEUCYL-TRNA S | 2.17 |
| 112290 | R53940 | Hs. 26016 | ESTs | 2.17 |
| 126136 | H83353 | | yv82f02.r1 Soares melanocyte 2NbHM Homo | 2.17 |
| 121574 | AA412712 | Hs. 119325 | Huntingtin-interacting protein A | 2.17 |
| 118530 | N67900 | Hs. 118446 | ESTs | 2.16 |
| 132327 | AA203285 | Hs. 44892 | ESTs; Weakly similar to dJ733D15.1 [H.sa | 2.16 |
| 100564 | HG2239-HT2324 | | Potassium Channel Protein (Gb: Z11585) | 2.16 |
| 129376 | AA022622 | Hs. 13543 | ESTs; Weakly similar to hypothetical pro | 2.16 |
| 135317 | X86012 | Hs. 98602 | Human DNA sequence from intron 22 of the | 2.16 |
| | | | 9.5 kb repeated region; int22h-1; involv | |
| 114973 | AA250845 | Hs. 87762 | ESTs | 2.16 |
| 107559 | AA001504 | Hs. 59860 | ESTs | 2.16 |
| 111014 | N53787 | Hs. 191117 | ESTs | 2.16 |
| 101250 | L34060 | Hs. 79133 | cadherin 8 | 2.16 |
| 110697 | H93721 | Hs. 20798 | ESTs | 2.16 |
| 126843 | AA450166 | Hs. 22641 | ESTs; Moderately similar to predicted pr | 2.16 |
| 108272 | AA063616 | Hs. 43773 | ESTs | 2.16 |
| 125012 | T66935 | Hs. 104859 | ESTs | 2.16 |
| 111639 | R16101 | Hs. 140834 | EST | 2.15 |
| 123157 | AA488443 | Hs. 100426 | DKFZP564A063 protein | 2.15 |
| 102315 | U34252 | Hs. 2533 | aldehyde dehydrogenase 9 (gamma-aminobut | 2.15 |
| 131897 | AA287623 | Hs. 3426 | GTPase; human homolog of E. coli essenti | 2.15 |
| 121528 | AA412253 | Hs. 238909 | ESTs; Weakly similar to POLYPOSIS LOCUS | 2.15 |
| 122806 | AA460707 | Hs. 106397 | ESTs | 2.15 |
| 125727 | H00958 | Hs. 181641 | ESTs | 2.15 |
| 133279 | AA069571 | Hs. 6957 | Homo sapiens clone 24616 mRNA sequence | 2.15 |
| 103219 | X74570 | Hs. 75268 | sialyltransferase 4C (beta-galactosidase | 2.15 |
| 120881 | AA362144 | Hs. 104601 | EST | 2.15 |
| 134060 | D42039 | Hs. 78871 | KIAA0081 protein | 2.15 |
| 106598 | AA457140 | Hs. 11411 | DKFZP566O084 protein | 2.15 |
| 125576 | R66208 | | yi30h03.r1 Soares placenta Nb2HP H sapie | 2.15 |
| | | | contains Alu repetitive element; contain | |
| 126727 | AA037230 | Hs. 135084 | cystatin C (amyloid angiopathy and cereb | 2.15 |
| 101490 | M25629 | Hs. 123107 | kallikrein 1; renal/pancreas/salivary | 2.15 |
| 129708 | AA417181 | Hs. 120858 | ESTs | 2.14 |
| 100627 | HG2702-HT2798 | | Serine/Threonine Kinase (Gb: Z25424) | 2.14 |
| 121703 | AA418671 | Hs. 104807 | ESTs | 2.14 |
| 106809 | AA479704 | Hs. 220324 | Humn DNA seq frm clone 283E3 on chr 1p36 | 2.14 |
| | | | Female Reproductive tract MIFR1; -2; MM | |
| 129525 | F03873 | Hs. 112306 | Homo sapiens clone 24955 mRNA sequence; | 2.14 |
| 100478 | HG1067-HT1067 | | Mucin (Gb: M22406) | 2.14 |
| 118593 | N69020 | Hs. 207689 | EST | 2.14 |
| 114047 | W94427 | Hs. 3807 | ESTs; Weakly similar to PHOSPHOLEMMAN PR | 2.14 |
| 128823 | AA478207 | Hs. 10632 | ESTs; Moderately similar to sex-determin | 2.14 |
| 100534 | HG1980-HT2023 | | Tubulin, Beta 2 | 2.14 |
| 105757 | AA321146 | Hs. 30596 | ESTs | 2.14 |
| 109617 | F03192 | Hs. 26789 | ESTs; Weakly similar to dJ162H14.1 [H.sa | 2.14 |
| 121547 | AA412448 | Hs. 104777 | ESTs | 2.14 |
| 119420 | T98291 | Hs. 102484 | glutathione S-transferaseA3 | 2.14 |
| 120274 | AA177051 | | nc02a02.s1 NCI_CGAP_Pr3 Homo sapiens cDN | 2.14 |
| | | | repetitive element;contains element LTR | |
| 132933 | AA598702 | Hs. 6101 | bone morphogenetic protein 6 | 2.14 |
| 133405 | X07881 | Hs. 73031 | proline-rich protein BstNI subfamily 3 | 2.14 |
| 119811 | W73922 | Hs. 49047 | ESTs | 2.14 |
| 134536 | AA457735 | Hs. 850 | IMP (inosine monophosphate) dehydrogenas | 2.14 |
| 105125 | AA157799 | Hs. 6980 | aldo-keto reductase family 7; member A2 | 2.14 |
| 101398 | M15881 | Hs. 1137 | uromodulin (uromucoid;Tamm-Horsfall gly | 2.14 |
| 132751 | AA397901 | Hs. 55993 | ESTs | 2.13 |
| 115777 | AA424142 | Hs. 39384 | putative secreted ligand homologous to f | 2.13 |
| 123193 | AA489228 | Hs. 136956 | ESTs | 2.13 |
| 116875 | H67749 | Hs. 161022 | EST | 2.13 |
| 107271 | D60607 | Hs. 34931 | EST | 2.13 |
| 134551 | R44839 | Hs. 8526 | i-beta-1; 3-N-acetylglucosaminyltransfera | 2.13 |
| 113413 | T83739 | Hs. 186512 | ESTs | 2.13 |
| 120522 | AA258843 | Hs. 258748 | ESTs | 2.13 |
| 119965 | W87738 | Hs. 59039 | EST | 2.13 |
| 131283 | AA101601 | Hs. 183986 | herpesvirus entry mediator B (poliovirus | 2.13 |
| 107347 | U43628 | Hs. 102598 | mucosal vascular addressin cell adhesion | 2.13 |
| 116490 | C14265 | Hs. 66450 | ESTs | 2.13 |
| 100563 | HG2239-HT2324 | | Potassium Channel Protein (Gb: Z11585) | 2.13 |
| 110441 | H50302 | Hs. 19845 | ESTs; Highly similar to protein phosphat | 2.13 |
| 101035 | J05158 | Hs. 73858 | carboxypeptidase N; polypeptide 2; 83 kD | 2.13 |
| 132500 | AA047297 | Hs. 50107 | ESTs; Moderately similar to CDO [H.sapie | 2.13 |
| 129807 | L34820 | Hs. 5299 | aldehyde dehydrogenase 5 family; member | 2.13 |
| 106250 | AA430466 | Hs. 28890 | ESTs | 2.13 |
| 113569 | T91086 | Hs. 162070 | EST | 2.13 |
| 122911 | AA470087 | Hs. 239726 | ESTs | 2.13 |
| 107452 | W28988 | Hs. 250746 | ESTs | 2.12 |
| 111824 | R35661 | Hs. 25006 | EST | 2.12 |
| 132831 | U53442 | Hs. 57732 | mitogen-activated protein kinase 11 | 2.12 |
| 110244 | H26742 | Hs. 25367 | ESTs; Weakly similar to ALR [H. sapiens] | 2.12 |
| 128918 | H85347 | Hs. 107164 | spectrin; beta; non-erythrocytic 1 | 2.12 |
| 133728 | M10901 | Hs. 75772 | nuclear receptor subfamily 3; group C; m | 2.12 |
| 122476 | AA448211 | Hs. 99164 | ESTs | 2.12 |
| 132004 | L37360 | Hs. 37054 | ephrin-A3 | 2.12 |
| 113971 | W86760 | Hs. 220682 | ESTs | 2.12 |
| 103386 | X92972 | Hs. 80324 | protein phosphatase 6; catalytic subunit | 2.12 |
| 131120 | AA443676 | Hs. 23133 | ESTs; Weakly similar to alcohol sulfotra | 2.12 |
| 102186 | U20285 | | G protein pathway suppressor 1 | 2.12 |
| 103694 | AA018541 | Hs. 60580 | zinc finger protein | 2.12 |
| 111995 | R42333 | Hs. 20893 | ESTs | 2.12 |
| 124436 | N39596 | Hs. 182584 | ESTs | 2.12 |
| 100306 | D50495 | Hs. 80598 | transcription elongation factor A (SII); | 2.12 |
| 103084 | X59932 | Hs. 77793 | c-src tyrosine kinase | 2.11 |
| 115092 | AA255903 | Hs. 80975 | CD39-like 4 | 2.11 |
| 121579 | AA416543 | Hs. 111981 | ESTs | 2.11 |
| 127101 | AI349351 | Hs. 118944 | ESTs | 2.11 |
| 121195 | AA400273 | Hs. 97791 | ESTs | 2.11 |
| 112721 | R91484 | Hs. 30853 | ESTs | 2.11 |
| 113253 | T64207 | Hs. 55296 | HLA-B associated transcript-1 | 2.11 |
| 120838 | AA348887 | Hs. 96907 | ESTs | 2.11 |
| 114122 | Z38582 | Hs. 12751 | ESTs | 2.11 |
| 112635 | R82298 | Hs. 29497 | ESTs | 2.11 |
| 103785 | AA095600 | Hs. 225647 | ESTs | 2.11 |
| 128260 | AA331445 | | EST35277 Embryo, 8 week I Homo sapiens c | 2.11 |
| 122987 | AA479155 | Hs. 103364 | ESTs | 2.11 |
| 110374 | H42983 | Hs. 227263 | ESTs | 2.11 |
| 116595 | D60625 | Hs. 177656 | calmodulin 1 (phosphorylase kinase; delt | 2.11 |
| 126117 | H78617 | | yu26a08.r1 Soares fetal liver spleen 1NF | 2.11 |
| 116610 | D80448 | Hs. 45177 | ESTs | 2.11 |
| 111430 | R01248 | Hs. 19165 | ESTs | 2.11 |
| 106700 | AA463929 | Hs. 28701 | ESTs | 2.11 |
| 120181 | Z40121 | Hs. 65870 | ESTs; Weakly similar to Pro-Pol-dUTPase | 2.1 |
| 132545 | AA147218 | Hs. 5105 | ESTs | 2.1 |
| 105005 | AA115253 | Hs. 28805 | ESTs | 2.1 |
| 126702 | U54602 | Hs. 2785 | keratin 17 | 2.1 |
| 124096 | H10060 | Hs. 101687 | EST | 2.1 |
| 132720 | Z69881 | Hs. 5541 | ATPase; Ca++ transporting; ubiquitous | 2.1 |
| 121926 | AA428559 | Hs. 104895 | ESTs | 2.1 |
| 125734 | AA157445 | Hs. 227391 | DKFZP547E1010 protein | 2.1 |
| 122368 | AA443963 | Hs. 104964 | EST | 2.1 |
| 116910 | H72014 | Hs. 161031 | ESTs; Weakly similar to SYNAPTOTAGMIN I | 2.1 |
| 113171 | T54613 | Hs. 9761 | EST | 2.1 |
| 134629 | U00951 | Hs. 87150 | Human clone A9A2BR11 (CAC)n/(GTG)n repea | 2.1 |
| 105712 | AA291293 | Hs. 25219 | ESTs | 2.1 |
| 106931 | AA495918 | Hs. 26714 | ESTs | 2.1 |
| 114278 | Z40424 | Hs. 27728 | ESTs | 2.1 |
| 116615 | D80666 | Hs. 45203 | ESTs | 2.09 |
| 100189 | D21089 | Hs. 320 | xeroderma pigmentosum; complementation g | 2.09 |
| 119500 | W37694 | Hs. 55561 | ESTs | 2.09 |
| 129605 | S72493 | Hs. 115947 | keratin 16 (focal non-epidermolytic palm | 2.09 |
| 133912 | X62744 | Hs. 77522 | major histocompatibility complex; class | 2.09 |
| 129636 | N34942 | Hs. 11782 | ESTs | 2.09 |
| 106372 | AA443941 | Hs. 4992 | tumor suppressing subtransferable candid | 2.09 |
| 101885 | M98539 | Hs. 8272 | prostaglandin D2 synthase (21 kD; brain) | 2.09 |
| 132749 | AA235989 | Hs. 55967 | short stature homeobox 2 | 2.09 |
| 135042 | X91348 | Hs. 93522 | putative non-coding transcript (DiGeorge | 2.09 |
| 109404 | AA224594 | Hs. 86941 | ESTs | 2.09 |
| 101333 | L47738 | Hs. 80313 | p53 inducible protein | 2.09 |
| 100114 | D00596 | Hs. 82962 | thymidylate synthetase | 2.09 |
| 130536 | T17045 | Hs. 159492 | spastic ataxia of Charlevoix-Saguenay (s | 2.09 |
| 125772 | R83903 | Hs. 78040 | KDEL (Lys-Asp-Glu-Leu) endoplasmic retic | 2.09 |
| 132192 | AA247569 | Hs. 4209 | ESTs | 2.09 |
| 124697 | R06273 | Hs. 186467 | ESTs; Moderately similar to !!!! ALU SUB | 2.09 |
| 127694 | AI247780 | Hs. 117036 | ESTs | 2.08 |
| 127895 | AA772600 | Hs. 187998 | ESTs; Weakly similar to ATP-binding cass | 2.08 |
| 121315 | AA402883 | Hs. 82269 | progestagen-associated endometrial prote | 2.08 |
| | | | endometrial alpha-2-globulin; alpha ute | |
| 112150 | R46576 | Hs. 23239 | ESTs | 2.08 |
| 105054 | AA133584 | Hs. 26333 | JM1 protein | 2.08 |
| 113151 | T51620 | Hs. 9326 | EST | 2.08 |
| 118783 | N75285 | Hs. 50593 | ESTs; Moderately similar to cytoplasmic | 2.08 |
| 126748 | AA249580 | Hs. 239975 | ESTs; Moderately similar to CDO [H.sapie | 2.08 |
| 135160 | U77643 | Hs. 95655 | secreted and transmembrane 1 | 2.08 |
| 107518 | X60152 | | zinc finger protein 2 | 2.08 |
| 126055 | N28990 | | yx39g04.r1 Soares melanocyte 2NbHM Homo | 2.08 |
| 116982 | H81933 | Hs. 40317 | ESTs | 2.08 |
| 101756 | M77235 | Hs. 169331 | sodium channel; voltage-gated; type V; a | 2.08 |
| 116935 | H75763 | Hs. 53468 | ESTs | 2.08 |
| 118556 | N68408 | Hs. 194637 | Homo sapiens mRNA; cDNA DKFZp564D113 (fr | 2.08 |
| 129812 | L07807 | Hs. 166161 | dynamin 1 | 2.08 |
| 121946 | AA429411 | Hs. 104888 | ESTs | 2.08 |
| 133843 | AA489045 | Hs. 76691 | Homo sapiens clone 25100 mRNA sequence; | 2.08 |
| 122170 | AA435744 | Hs. 163913 | ESTs | 2.08 |
| 122399 | AA446449 | Hs. 231112 | EST | 2.08 |
| 105775 | AA348274 | Hs. 6664 | ESTs | 2.08 |
| 123943 | AA621553 | Hs. 112998 | ESTs | 2.08 |
| 105771 | AA347967 | Hs. 256267 | neuroblastoma RAS viral (v-ras) oncogene | 2.08 |
| 114454 | AA021091 | Hs. 226208 | ESTs | 2.08 |
| 125802 | R78852 | Hs. 151099 | ESTs | 2.08 |
| 131556 | AA442853 | Hs. 2869 | cyclin-dependent kinase 5; regulatory su | 2.08 |
| 118837 | N79836 | Hs. 216338 | ESTs | 2.08 |
| 107345 | U26209 | Hs. 102307 | solute carrier family 13 (sodium-depende | 2.08 |
| 131324 | H58690 | Hs. 25625 | ESTs | 2.08 |
| 105233 | AA216759 | Hs. 191132 | ESTs | 2.07 |
| 112886 | T03864 | Hs. 7436 | putative acyltransferase | 2.07 |
| 120252 | AA169400 | Hs. 152701 | DKFZP434F124 protein | 2.07 |
| 114867 | AA235310 | Hs. 52899 | ESTs; Moderately similar to !!!! ALU SUB | 2.07 |
| 106715 | AA464955 | Hs. 126062 | ESTs; Weakly similar to EPIDERMAL GROWTH | 2.07 |
| 125560 | R51281 | Hs. 13692 | ESTs; Highly similar to PROTEIN TSG24 [M | 2.07 |
| 112270 | R53021 | Hs. 203358 | ESTs | 2.07 |
| 134626 | S82198 | Hs. 8709 | caldecrin (serum calcium decreasing fact | 2.07 |
| 115723 | AA417345 | Hs. 54846 | ESTs | 2.07 |
| 123895 | AA621192 | Hs. 112949 | EST | 2.07 |
| 119906 | W85818 | | ESTs; Moderately similar to !!!! ALU SUB | 2.07 |
| 108559 | AA085161 | | zn12c5.s1 Stratagene hNT neuron (#937233 | 2.07 |
| | | | IMAGE: 54728 3′ similar to TR: G1151228 G | |
| 101246 | L33799 | Hs. 202097 | procollagen C-endopeptidase enhancer | 2.07 |
| 100663 | HG2915-HT3059 | | Major Histocompatibility Complex, Class I, E (Gb: M20022) | 2.07 |
| 114178 | Z39063 | Hs. 17930 | Humn DNA seq frm clone 1033B10 on chr 6p | 2.07 |
| | | | for GalT3 (beta3-Galactosyltransferase) | |
| 125672 | AA152281 | Hs. 78601 | uroporphyrinogen decarboxylase | 2.07 |
| 118052 | N53360 | Hs. 165133 | ESTs | 2.07 |
| 102387 | U41163 | Hs. 229731 | solute carrier family 6 (neurotransmitte | 2.07 |
| 127305 | AA535148 | Hs. 255277 | ESTs | 2.07 |
| 101182 | L19711 | Hs. 76111 | dystroglycan 1 (dystrophin-associated gl | 2.07 |
| 131111 | R33245 | Hs. 23076 | ESTs; Weakly similar to putative [C.eleg | 2.07 |
| 112441 | R63388 | Hs. 28412 | ESTs | 2.06 |
| 117796 | N48571 | Hs. 46689 | EST | 2.06 |
| 116099 | AA456309 | Hs. 58831 | regulator of Fas-induced apoptosis | 2.06 |
| 125559 | AA307550 | Hs. 119571 | collagen; type III; alpha 1 (Ehlers-Danl | 2.06 |
| 135271 | AA397763 | Hs. 97562 | ESTs | 2.06 |
| 106083 | AA418545 | Hs. 31659 | thyroid hormone receptor-associated prot | 2.06 |
| 133419 | U67369 | Hs. 73172 | growth factor independent 1 | 2.06 |
| 127816 | AA743646 | Hs. 120604 | ESTs | 2.06 |
| 127502 | AA614422 | Hs. 183502 | ESTs | 2.06 |
| 129371 | M10321 | Hs. 110802 | von Willebrand factor | 2.06 |
| 108417 | AA075716 | | zm89e5.s1 Stratagene ovarian cancer (#93 | 2.06 |
| | | | CLUSTERIN PRECURSOR (HUMAN);, mRNA sequ | |
| 102837 | U94585 | Hs. 13495 | requiem; apoptosis response zinc finger | 2.06 |
| 124226 | H62396 | Hs. 190266 | ESTs | 2.06 |
| 102254 | U28131 | | Human HMGI-C chimeric transcript mRNA, p | 2.06 |
| 128472 | X87212 | Hs. 10029 | cathepsin C | 2.06 |
| 107545 | Z82022 | Hs. 26433 | dolichyl-phosphate (UDP-N-acetylglucosam | 2.06 |
| 135311 | M36089 | Hs. 98493 | X-ray repair complementing defective rep | 2.06 |
| 121727 | AA420973 | Hs. 104234 | ESTs | 2.06 |
| 131846 | U02619 | Hs. 331 | general transcription factor IIIC; polyp | 2.06 |
| 120415 | AA235810 | Hs. 182522 | ESTs | 2.06 |
| 110529 | H57686 | Hs. 37486 | ESTs | 2.06 |
| 104996 | AA112307 | Hs. 105894 | Homo sapiens mRNA; cDNA DKFZp434G231 (fr | 2.06 |
| 110351 | H41222 | Hs. 196459 | ESTs | 2.06 |
| 131261 | AA223746 | Hs. 171776 | inositol(myo)-1(or 4)-monophosphatase 1 | 2.06 |
| 110585 | H62223 | Hs. 133526 | ESTs; Weakly similar to !!!! ALU SUBFAMI | 2.06 |
| 129420 | AA234259 | Hs. 99816 | ESTs | 2.06 |
| 103796 | AA112595 | Hs. 31146 | Human DNA sequence from clone 1042K10 on | 2.06 |
| | | | lyase (EC 4.3.2.2; Adenylosuccinase; AS | |
| | | | 3). Contains ESTs; STSs; GS | |
| 119782 | W72982 | Hs. 58262 | ESTs | 2.06 |
| 108641 | AA112059 | | ATP synthase; H+ transporting; mitochond | 2.06 |
| 134875 | U66672 | Hs. 180513 | ATP-binding cassette; sub-family A (ABC1 | 2.06 |
| 106832 | AA482015 | Hs. 30114 | ESTs; Highly similar to C8 [H. sapiens] | 2.06 |
| 109403 | AA224413 | Hs. 86937 | ESTs | 2.06 |
| 115485 | AA287667 | Hs. 188804 | ESTs | 2.06 |
| 102923 | X12517 | Hs. 1063 | small nuclear ribonucleoprotein polypept | 2.06 |
| 123320 | AA496792 | Hs. 139572 | EST | 2.05 |
| 111901 | R39066 | Hs. 17638 | ESTs | 2.05 |
| 106558 | AA455111 | Hs. 182447 | heterogeneous nuclear ribonucleoprotein | 2.05 |
| 126885 | AA293052 | Hs. 10101 | ESTs; Weakly similar to coded for by C. | 2.05 |
| 113429 | T85190 | Hs. 179808 | ESTs | 2.05 |
| 102270 | U30255 | Hs. 75888 | phosphogluconate dehydrogenase | 2.05 |
| 103204 | X72475 | Hs. 192989 | H. sapiens mRNA for rearranged Ig kappa I | 2.05 |
| 106666 | AA461072 | Hs. 37916 | ESTs | 2.05 |
| 100947 | HG907-HT907 | | Mg44 | 2.05 |
| 102578 | U60666 | Hs. 57693 | testis specific leucine rich repeat prot | 2.05 |
| 105827 | AA398255 | Hs. 31520 | ESTs | 2.05 |
| 122324 | AA442830 | Hs. 98921 | EST | 2.05 |
| 101025 | J04823 | Hs. 81097 | cytochrome c oxidase subunit VIII | 2.05 |
| 115861 | AA431768 | Hs. 90259 | ESTs; Weakly similar to alpha 1 [H.sapie | 2.05 |
| 108081 | AA045306 | Hs. 42996 | ESTs | 2.05 |
| 133994 | X74929 | Hs. 242463 | keratin 8 | 2.05 |
| 119131 | R46700 | Hs. 129692 | ESTs; Moderately similar to !!!! ALU SUB | 2.05 |
| 129793 | AA300151 | Hs. 126857 | ESTs | 2.05 |
| 101653 | M60284 | Hs. 161305 | tachykinin receptor 2 | 2.05 |
| 120300 | AA191648 | Hs. 131476 | ESTs | 2.05 |
| 106519 | AA453415 | Hs. 8763 | Hu DNA sequence from clone 889N15 on chr | 2.05 |
| | | | Thymocyte Marker CTX; the possibly alte | |
| 114291 | Z40690 | Hs. 123666 | Homo sapiens mRNA full length insert cDN | 2.05 |
| 105747 | AA293719 | Hs. 30251 | ESTs; Weakly similar to GLUCOSE-6-PHOSPH | 2.04 |
| 125325 | AA332944 | Hs. 8402 | adenylate cyclase 3 | 2.04 |
| 119978 | W88623 | Hs. 59190 | EST | 2.04 |
| 102449 | U48231 | Hs. 46348 | bradykinin receptor B1 | 2.04 |
| 101454 | M21812 | Hs. 50889 | myosin light chain 2 | 2.04 |
| 116086 | AA455904 | Hs. 86023 | ESTs | 2.04 |
| 102297 | U32674 | Hs. 198252 | G protein-coupled receptor 9 | 2.04 |
| 130889 | D57622 | Hs. 20985 | sin3-associated polypeptide; 30 kD | 2.04 |
| 100196 | D21853 | Hs. 79768 | KIAA0111 gene product | 2.04 |
| 120967 | AA398111 | Hs. 97503 | ESTs | 2.04 |
| 105735 | AA293096 | Hs. 32417 | ESTs | 2.04 |
| 135031 | R41604 | Hs. 9344 | ESTs; Weakly similar to !!!! ALU SUBFAMI | 2.04 |
| 104882 | AA052954 | Hs. 29546 | ESTs | 2.04 |
| 132619 | AA404565 | Hs. 53447 | ESTs; Moderately similar to kinesin ligh | 2.04 |
| 127993 | AA847856 | Hs. 124565 | ESTs | 2.04 |
| 116441 | AA620299 | Hs. 91696 | ESTs | 2.04 |
| 102272 | U30610 | Hs. 41682 | killer cell lectin-like receptor subfami | 2.04 |
| 119566 | W38209 | | Accession not listed in Genbank | 2.04 |
| 116622 | D81171 | Hs. 45208 | ESTs; Weakly similar to collagen type VI | 2.04 |
| 127182 | AA248620 | Hs. 166011 | catenin (cadherin-associated protein); d | 2.04 |
| 116870 | H67146 | Hs. 38564 | ESTs | 2.04 |
| 115448 | AA284845 | Hs. 165051 | ESTs | 2.04 |
| 127231 | AA434584 | | zw52c03.r1 Soares_total_fetus_Nb2HF8_9w | 2.04 |
| 103457 | X99728 | | H. sapiens NDUFV3 gene, exon 3 | 2.04 |
| 134737 | U00802 | Hs. 89434 | drebrin 1 | 2.04 |
| 117046 | H89505 | | yu81f4.s1 Soares fetal liver spleen 1NFL | 2.04 |
| | | | to contains Alu repetitive element; mR | |
| 124579 | N68345 | Hs. 127179 | ESTs; Weakly similar to TERATOCARCINOMA- | 2.04 |
| 112132 | R45970 | Hs. 236349 | EST | 2.04 |
| 132281 | AA133300 | Hs. 43803 | leukocyte-associated Ig-like receptor 2 | 2.03 |
| 103668 | Z83741 | Hs. 248174 | H2A histone family; member M | 2.03 |
| 113501 | T89107 | Hs. 13262 | ESTs | 2.03 |
| 125021 | T70060 | Hs. 163918 | ESTs | 2.03 |
| 115754 | AA420998 | Hs. 178095 | ESTs | 2.03 |
| 123405 | AA521370 | Hs. 191708 | ESTs | 2.03 |
| 102054 | U07695 | Hs. 155227 | EphB4 | 2.03 |
| 115627 | AA401910 | Hs. 119175 | ESTs; Weakly similar to ZINC FINGER PROT | 2.03 |
| 129252 | AA234663 | Hs. 109773 | ESTs | 2.03 |
| 103417 | X96849 | | H. sapiens 5′ mRNA of PECAM-1 molecule | 2.03 |
| 133721 | U11863 | Hs. 75741 | amiloride binding protein 1 (amine oxida | 2.03 |
| 114176 | Z39059 | Hs. 27267 | ESTs; Weakly similar to tetraspan TM4SF | 2.03 |
| 123966 | C14068 | Hs. 21806 | ESTs; Moderately similar to similar to N | 2.03 |
| 134236 | D45371 | Hs. 80485 | adipose most abundant gene transcript 1 | 2.03 |
| 116381 | AA598614 | Hs. 65394 | ESTs | 2.03 |
| 103711 | AA046737 | Hs. 102792 | ESTs | 2.03 |
| 109316 | AA206914 | Hs. 86322 | EST | 2.03 |
| 123793 | AA620343 | Hs. 112858 | ESTs | 2.03 |
| 128462 | M69238 | Hs. 166172 | aryl hydrocarbon receptor nuclear transl | 2.03 |
| 117690 | N40467 | Hs. 93834 | ESTs | 2.03 |
| 113301 | T67452 | Hs. 13104 | EST | 2.03 |
| 134563 | AA173430 | Hs. 85335 | Home sapiens mRNA; cDNA DKFZp564D1462 (f | 2.03 |
| 108316 | AA070160 | | zm69f4.s1 Stratagene neuroepithelium (#9 | 2.03 |
| 135239 | AA454599 | Hs. 19399 | Homo sapiens chromosome 19; fosmid 39554 | 2.03 |
| 120342 | AA207105 | Hs. 45068 | Home sapiens mRNA; cDNA DKFZp434I143 (fr | 2.02 |
| 103493 | Y08976 | Hs. 234759 | H. sapiens mRNA for FEV protein | 2.02 |
| 114204 | Z39259 | Hs. 26096 | ESTs | 2.02 |
| 125425 | H62307 | Hs. 18575 | ESTs; Weakly similar to KIAA0246 [H.sapi | 2.02 |
| 133027 | AA402624 | Hs. 63236 | synuclein; gamma (breast cancer-specific | 2.02 |
| 131323 | H54036 | Hs. 25619 | death-associated protein kinase 3 | 2.02 |
| 121515 | AA412133 | Hs. 104696 | ESTs | 2.02 |
| 129780 | AA291526 | Hs. 124699 | ESTs | 2.02 |
| 131292 | AF005039 | Hs. 200600 | secretory carrier membrane protein 3 | 2.02 |
| 132973 | AA035446 | Hs. 214361 | ESTs | 2.02 |
| 103727 | AA059415 | Hs. 6289 | growth factor receptor-bound protein 2 | 2.02 |
| 113174 | T54659 | Hs. 9779 | ESTs | 2.02 |
| 120964 | AA398085 | Hs. 142390 | ESTs | 2.02 |
| 134303 | AA457242 | Hs. 8141 | etoposide-induced mRNA | 2.02 |
| 128118 | T81623 | Hs. 21765 | hypothetical protein of unknown functio | 2.02 |
| 121087 | AA398751 | Hs. 97304 | ESTs | 2.02 |
| 102806 | U90306 | | Human iroquois-class homeodomain protein | 2.02 |
| 103195 | X70940 | Hs. 2642 | eukaryotic translation elongation factor | 2.02 |
| 126767 | C17148 | | C17148 Clontech human aorta polyA+ mRNA | 2.02 |
| 105179 | AA189083 | Hs. 21974 | ESTs; Moderately similar to mBOCT [M.mus | 2.02 |
| 116797 | H40486 | | yn87a08.s1 Soares adult brain N2b5HB55Y | 2.02 |
| | | | 3′ similar to contains Alu repetitive e | |
| 133268 | AA099404 | Hs. 69307 | ESTs | 2.02 |
| 123951 | AA621721 | Hs. 231130 | EST | 2.02 |
| 115463 | AA286819 | Hs. 69485 | ESTs; Weakly similar to similar to other | 2.02 |
| 110603 | H65776 | Hs. 222403 | ESTs | 2.02 |
| 101234 | L29277 | Hs. 142258 | signal transducer and activator of trans | 2.02 |
| 121208 | AA400470 | Hs. 97805 | ESTs | 2.02 |
| 122598 | AA453465 | Hs. 99329 | ESTs | 2.02 |
| 110668 | H84882 | Hs. 33791 | ESTs; Weakly similar to K: Cl cotransport | 2.02 |
| 117137 | H96670 | Hs. 42221 | ESTs | 2.02 |
| 119389 | T88826 | Hs. 90973 | ESTs | 2.01 |
| 102940 | X13956 | Hs. 24998 | Human 12S RNA induced by poly(rl); poly( | 2.01 |
| 100748 | HG3517-HT3711 | | Alpha-1-Antitrypsin, 5′ End | 2.01 |
| 103012 | X52638 | Hs. 739 | 6-phosphofructo-2-kinase/fructose-2; 6-bi | 2.01 |
| 132755 | AA609201 | Hs. 182635 | ESTs | 2.01 |
| 130842 | H39589 | Hs. 20159 | ESTs; Highly similar to CGI-92 protein [ | 2.01 |
| 133599 | M64788 | Hs. 75151 | RAP1; GTPase activating protein 1 | 2.01 |
| 117250 | N21081 | Hs. 15299 | HMBA-inducible | 2.01 |
| 115124 | AA256666 | Hs. 39156 | ESTs | 2.01 |
| 128155 | AA926843 | Hs. 143302 | ESTs | 2.01 |
| 130574 | AA379087 | Hs. 16178 | apoptosis antagonizing transcription fac | 2.01 |
| 132601 | R78838 | Hs. 54943 | fracture callus 1 (rat) homolog | 2.01 |
| 117428 | N27366 | Hs. 43933 | EST | 2.01 |
| 121108 | AA399053 | Hs. 97529 | EST | 2.01 |
| 130518 | X69550 | Hs. 159161 | Rho GDP dissociation inhibitor (GDI) alp | 2.01 |
| 110606 | H66049 | Hs. 19085 | ESTs; Weakly similar to putative p150 [H | 2.01 |
| 120606 | AA282956 | | zt15h4.s1 NCI_CGAP_GCB1 Homo sapiens cDN | |
| | | | SW: CADR_MOUSE P3938 RETINAL-CADHERIN PR | 2.01 |
| 130070 | T47969 | Hs. 194660 | ceroid-lipofuscinosis; neuronal 3; juven | 2.01 |
| 130331 | Z80783 | Hs. 239884 | H2B histone family; member L | 2.01 |
| 109599 | F02602 | Hs. 6749 | ESTs | 2.01 |
| 131749 | W78211 | Hs. 31547 | ESTs; Highly similar to NADH: ubiquinone | 2.01 |
| 129463 | AA376905 | Hs. 111742 | ESTs; Weakly similar to !!!! ALU SUBFAMI | 2.01 |
| 114880 | AA235698 | Hs. 65862 | ESTs | 2.01 |
| 114745 | AA135523 | Hs. 139064 | EST | 2.01 |
| 115637 | AA402727 | Hs. 76925 | ESTs; Highly similar to R31167_2; partia | 2.01 |
| 109043 | AA159605 | Hs. 72580 | ESTs | 2.01 |
| 128901 | Z41411 | Hs. 107040 | ESTs | 2.01 |
| 124427 | N36812 | Hs. 178663 | ESTs | 2 |
| 100673 | HG3033-HT3194 | | Spliceosomal Protein Sap 62 | 2 |
| 108436 | AA078801 | | zm94a9.s1 Stratagene colon HT29 (#937221 | 2 |
| 123764 | AA610019 | Hs. 112654 | ESTs | 2 |
| 129343 | N70791 | Hs. 180060 | ESTs | 2 |
| 122794 | AA460254 | Hs. 105043 | EST | 2 |
| 128688 | AA161469 | Hs. 103755 | receptor-interacting serine-threonine ki | 2 |
| 115592 | AA399543 | Hs. 48026 | ESTs | 2 |
| 111693 | R22007 | Hs. 23321 | EST | 2 |
| 113353 | T79186 | Hs. 14468 | ESTs | 2 |

Pkey: Unique Eos probeset identifier number
ExAccn: Exemplar Accession number, Genbank accession number
UnigeneID: Unigene number
Unigene Title: Unigene gene title

TABLE 18
B survivor vs Mets - Up in Mets

| Pkey | Ex Accn | UniG ID | Complete Title | Ratio BS/Met |
|---|---|---|---|---|
| 106024 | AA412059 | Hs. 111742 | ESTs; Weakly similar to !!!! ALU SUBFAMI | 0.17 |
| 110930 | N48603 | Hs. 14947 | ESTs | 0.18 |
| 105772 | AA347973 | Hs. 221132 | ESTs | 0.2 |
| 133271 | Z48633 | Hs. 6940 | H. sapiens mRNA for retrotransposon | 0.2 |
| 107109 | AA609943 | Hs. 32793 | ESTs | 0.24 |
| 109593 | F02506 | Hs. 159591 | thyroid hormone receptor interactor 8 | 0.24 |
| 123016 | AA480103 | Hs. 111730 | ESTs; Weakly similar to alternatively sp | 0.25 |
| 100739 | HG3484-HT3678 | | Protein Kinase (Gb: M59287) | 0.25 |
| 130252 | U92014 | Hs. 153527 | Human clone 121711 defective mariner tra | 0.26 |
| 105149 | AA169253 | Hs. 8958 | ESTs | 0.26 |
| 115412 | AA283804 | Hs. 193552 | ESTs | 0.27 |
| 105952 | AA405263 | Hs. 181400 | ESTs | 0.28 |
| 106596 | AA456981 | Hs. 35349 | ESTs | 0.28 |
| 120249 | AA167567 | Hs. 133325 | ESTs | 0.28 |
| 111676 | R19414 | Hs. 166459 | ESTs | 0.29 |
| 111161 | N66767 | Hs. 124145 | ESTs | 0.29 |
| 109364 | AA215379 | Hs. 50418 | ESTs | 0.29 |
| 132316 | U28831 | | Human protein immuno-reactive with anti- | 0.3 |
| 104030 | AA363131 | Hs. 222992 | ESTs; Weakly similar to TRANSFORMATION-S | 0.3 |
| 109825 | F13663 | Hs. 16798 | ESTs | 0.3 |
| 111110 | N63165 | Hs. 23618 | ESTs | 0.31 |
| 135315 | W90583 | Hs. 9853 | ESTs | 0.32 |
| 104792 | AA029288 | Hs. 29147 | ESTs; Highly similar to ZINC FINGER PROT | 0.33 |
| 123562 | AA608893 | Hs. 190065 | ESTs | 0.33 |
| 116079 | AA455286 | Hs. 54982 | ESTs; Weakly similar to !!!! ALU SUBFAMI | 0.33 |
| 110671 | H87770 | Hs. 153800 | ESTs | 0.33 |
| 108819 | AA130986 | Hs. 193253 | ESTs | 0.34 |
| 115558 | AA393806 | Hs. 1010 | regulator of mitotic spindle assembly 1 | 0.34 |
| 104781 | AA026617 | Hs. 21610 | ESTs; Highly similar to BAI1-associated | 0.34 |
| 111236 | N69324 | Hs. 12526 | Homo sapiens clone 23903 mRNA sequence | 0.34 |
| 113341 | T77866 | Hs. 189703 | ESTs | 0.35 |
| 125371 | AI084676 | Hs. 133266 | ESTs; Moderately similar to Sqv-7-like p | 0.35 |
| 115890 | AA435853 | Hs. 44114 | ESTs; Weakly similar to CGI-73 protein [ | 0.35 |
| 113571 | T91116 | Hs. 15713 | ESTs | 0.35 |
| 121683 | AA417911 | Hs. 175663 | ESTs | 0.35 |
| 105489 | AA256157 | Hs. 24115 | ESTs | 0.35 |
| 116320 | AA490866 | Hs. 39429 | ESTs | 0.36 |
| 111917 | R39882 | Hs. 21397 | ESTs | 0.36 |
| 127568 | T53722 | | ya91c06.r3 Stratagene placenta (#937225) | 0.36 |
| 123541 | AA608794 | Hs. 112592 | ESTs | 0.36 |
| 123131 | AA487207 | Hs. 193272 | ESTs | 0.36 |
| 125069 | T86914 | Hs. 194485 | ESTs | 0.36 |
| 114757 | AA136725 | Hs. 161990 | ESTs | 0.37 |
| 132778 | AA446695 | Hs. 5671 | Homo sapiens clone 23926 mRNA sequence | 0.37 |
| 123132 | AA487233 | Hs. 106711 | eukaryotic translation initiation factor | 0.37 |
| 134029 | AA378597 | Hs. 143601 | ESTs; Moderately similar to 67A9.b [D.me | 0.37 |
| 126956 | AI434405 | Hs. 171957 | triple functional domain (PTPRF interact | 0.38 |
| 106869 | AA487563 | Hs. 188813 | ESTs | 0.38 |
| 107818 | AA020957 | Hs. 167948 | ESTs | 0.38 |
| 129974 | K00629 | Hs. 199300 | Human kpni repeat mma (cdna clone pcd-k | 0.38 |
| 129477 | D49728 | Hs. 1119 | nuclear receptor subfamily 4; group A; m | 0.38 |
| 119369 | T79020 | Hs. 245915 | ESTs; Weakly similar to kinase-related p | 0.39 |
| 114021 | W91995 | Hs. 16145 | ESTs | 0.39 |
| 122024 | AA431296 | Hs. 139433 | EST | 0.39 |
| 130014 | N50959 | Hs. 143102 | amine oxidase; copper containing 2 (reti | 0.39 |
| 110163 | H19326 | Hs. 22073 | ESTs; Highly similar to J KAPPA-RECOMBIN | 0.39 |
| 104641 | AA004652 | Hs. 18564 | ESTs | 0.39 |
| 124777 | R41933 | Hs. 140237 | ESTs | 0.39 |
| 125382 | AA713494 | Hs. 194660 | ceroid-lipofuscinosis; neuronal 3; juven | 0.4 |
| 120406 | AA234999 | Hs. 111279 | ESTs; Weakly similar to unnamed protein | 0.4 |
| 132734 | R23653 | Hs. 164250 | ESTs | 0.4 |
| 117001 | H84719 | Hs. 40721 | EST | 0.4 |
| 120905 | AA371602 | Hs. 182930 | ESTs; Highly similar to PHOSPHATIDYLINOS | 0.4 |
| 125488 | AA355158 | Hs. 41181 | Homo sapiens mRNA; cDNA DKFZp727C191 (fr | 0.4 |
| 121989 | AA430044 | Hs. 193784 | Homo sapiens mRNA; cDNA DKFZp586K1922 (f | 0.4 |
| 127921 | AA806616 | Hs. 209523 | ESTs | 0.4 |
| 119830 | W74700 | Hs. 53478 | ESTs | 0.41 |
| 106292 | AA435571 | Hs. 148560 | ESTs | 0.41 |
| 102762 | U82303 | Hs. 123080 | Homo sapiens unknown protein mRNA; parti | 0.41 |
| 113518 | T89731 | | ye11f06.s1 Stratagene lung (#937210) H.s | |
| | | | to contains Alu repetitive element;cont | 0.41 |
| 100635 | HG2724-HT2820 | | Oncogene Tls/Chop, Fusion Activated | 0.41 |
| 113319 | T70356 | Hs. 193141 | ESTs; Weakly similar to coding sequence | 0.41 |
| 121319 | AA402935 | Hs. 194242 | ESTs; Weakly similar to !!!! ALU CLASS B | 0.42 |
| 111818 | R34382 | Hs. 24779 | ESTs | 0.42 |
| 104883 | AA052959 | Hs. 177409 | ESTs; Highly similar to dJ1119D9.2 [H.sa | 0.42 |
| 129258 | W95592 | Hs. 251946 | ESTs; Moderately similar to POLYADENYLAT | 0.42 |
| 130576 | T86475 | Hs. 16193 | Homo sapiens mRNA; cDNA DKFZp586B211 (fr | 0.43 |
| 106354 | AA443271 | Hs. 26764 | KIAA0546 protein | 0.43 |
| 108841 | AA132524 | Hs. 70614 | ESTs | 0.43 |
| 113922 | W80741 | Hs. 37890 | ESTs | 0.43 |
| 120997 | AA398285 | Hs. 97598 | EST | 0.43 |
| 108158 | AA054597 | Hs. 221935 | ESTs | 0.43 |
| 124516 | N58185 | Hs. 131830 | ESTs | 0.43 |
| 114477 | AA032013 | Hs. 144260 | EST | 0.43 |
| 104290 | C16652 | Hs. 107205 | Homo sapiens mRNA; cDNA DKFZp434L2221 (f | 0.43 |
| 126700 | AI318412 | Hs. 108258 | actin binding protein; macrophin (microf | 0.44 |
| 110887 | N38770 | Hs. 4283 | ESTs | 0.44 |
| 116141 | AA460420 | Hs. 44949 | ESTs | 0.44 |
| 110689 | H93046 | Hs. 15571 | ESTs | 0.44 |
| 115314 | AA280583 | Hs. 256501 | ESTs | 0.44 |
| 110904 | N39453 | Hs. 27371 | Homo sapiens mRNA; cDNA DKFZp566J123 (fr | 0.44 |
| 109482 | AA233375 | Hs. 78085 | ESTs | 0.44 |
| 102284 | U31449 | Hs. 11881 | transmembrane 4 superfamily member 4 | 0.44 |
| 118654 | N70582 | Hs. 49892 | ESTs | 0.44 |
| 115334 | AA281244 | Hs. 65300 | ESTs | 0.44 |
| 113149 | T51588 | | ESTs; Moderately similar to !!!! ALU SUB | 0.44 |
| 113721 | T97931 | Hs. 18190 | EST | 0.44 |
| 111299 | N73808 | Hs. 24936 | ESTs | 0.44 |
| 103778 | AA094107 | Hs. 7187 | ESTs; Weakly similar to similar to glyco | 0.44 |
| 113204 | T57865 | Hs. 10310 | EST | 0.44 |
| 100315 | D50857 | Hs. 82295 | dedicator of cyto-kinesis 1 | 0.44 |
| 115254 | AA279024 | Hs. 194437 | ESTs | 0.44 |
| 125500 | H46104 | Hs. 244624 | ESTs | 0.44 |
| 117387 | N26011 | Hs. 53810 | ESTs | 0.45 |
| 135113 | W42450 | Hs. 206833 | ESTs | 0.45 |
| 124517 | N58204 | Hs. 199945 | ESTs | 0.45 |
| 120379 | AA227849 | Hs. 238380 | Human endogenous retroviral protease mRN | 0.45 |
| 119205 | R91954 | Hs. 153699 | ESTs | 0.45 |
| 128266 | T70341 | Hs. 131897 | ESTs | 0.45 |
| 104106 | AA422123 | Hs. 42457 | ESTs | 0.45 |
| 115864 | AA432080 | Hs. 81200 | ESTs | 0.45 |
| 113771 | W02695 | Hs. 18714 | ESTs | 0.45 |
| 126515 | AI124649 | Hs. 252708 | Homo sapiens mRNA; cDNA DKFZp586O031 (fr | 0.45 |
| 127823 | AA524806 | Hs. 78869 | transcription elongation factor A (SII); | 0.45 |
| 116665 | F04405 | Hs. 223654 | EST | 0.45 |
| 106355 | AA443272 | Hs. 27836 | ESTs | 0.45 |
| 132693 | AA621429 | Hs. 55075 | KIAA0410 gene product | 0.45 |
| 107388 | W01587 | Hs. 173319 | ESTs | 0.45 |
| 110688 | H93021 | Hs. 182937 | peptidylprolyl isomerase A (cyclophilin | 0.46 |
| 116893 | H69569 | Hs. 191316 | EST | 0.46 |
| 105375 | AA236542 | Hs. 9512 | ESTs; Moderately similar to !!!! ALU SUB | 0.46 |
| 115601 | AA400277 | Hs. 48849 | ESTs | 0.46 |
| 106896 | AA489707 | Hs. 29896 | ESTs; Weakly similar to proline-rich pro | 0.46 |
| 111770 | R27975 | Hs. 187469 | ESTs | 0.46 |
| 115663 | AA405838 | Hs. 40507 | ESTs | 0.46 |
| 131404 | AA504744 | Hs. 26461 | ESTs; Weakly similar to gc-rich sequence | 0.46 |
| 108622 | AA101828 | Hs. 189956 | ESTs | 0.46 |
| 128286 | AI025771 | Hs. 144090 | ESTs | 0.46 |
| 105760 | AA338960 | Hs. 28170 | ESTs | 0.46 |
| 100020 | | | AFFX control: BioB-3 | 0.46 |
| 105209 | AA205072 | Hs. 227743 | KIAA0980 protein | 0.47 |
| 111975 | R41724 | Hs. 149566 | ESTs | 0.47 |
| 114688 | AA121403 | Hs. 144331 | ESTs | 0.47 |
| 116994 | H83918 | Hs. 40528 | ESTs | 0.47 |
| 118401 | N64762 | Hs. 49053 | EST | 0.47 |
| 110997 | N52540 | Hs. 74316 | desmoplakin (DPI; DPII) | 0.47 |
| 123791 | AA620331 | Hs. 245351 | EST | 0.47 |
| 109858 | H02266 | Hs. 167451 | ESTs | 0.47 |
| 115470 | AA287122 | Hs. 48391 | ESTs | 0.47 |
| 130606 | AA402109 | Hs. 16593 | ESTs | 0.47 |
| 116067 | AA454827 | Hs. 124823 | ESTs | 0.47 |
| 125881 | AA775807 | Hs. 150741 | 2′;3′-cyclic nucleotide 3′ phosphodieste | 0.47 |
| 124028 | F04112 | Hs. 177178 | ESTs | 0.47 |
| 108995 | AA155574 | Hs. 172702 | ESTs | 0.47 |
| 125102 | T95105 | Hs. 173772 | ESTs | 0.47 |
| 110421 | H48462 | Hs. 36093 | ESTs; Weakly similar to reverse transcri | 0.47 |
| 105658 | AA282914 | Hs. 10176 | ESTs | 0.47 |
| 129046 | AA195678 | Hs. 108258 | actin binding protein; macrophin (microf | 0.47 |
| 113639 | T95128 | Hs. 17529 | ESTs | 0.48 |
| 132575 | AA045365 | Hs. 5188 | ESTs; Weakly similar to 60S RIBOSOMAL PR | 0.48 |
| 132592 | AA129390 | Hs. 5285 | ESTs | 0.48 |
| 107619 | AA004955 | Hs. 60015 | ESTs | 0.48 |
| 118664 | N70907 | Hs. 230619 | EST | 0.48 |
| 127612 | AA917801 | Hs. 116076 | ESTs | 0.48 |
| 112319 | R55615 | Hs. 26432 | ESTs; Weakly similar to finger protein H | 0.48 |
| 113635 | T95087 | Hs. 15543 | ESTs | 0.48 |
| 119344 | T62969 | Hs. 193348 | ESTs | 0.48 |
| 121080 | AA398720 | Hs. 177953 | ESTs | 0.48 |
| 133686 | X83378 | Hs. 211614 | chloride channel 6 | 0.48 |
| 130395 | R54534 | Hs. 87889 | helicase-moi | 0.49 |
| 127530 | AA563806 | Hs. 145728 | ESTs | 0.49 |
| 132971 | AA033951 | Hs. 61700 | ESTs | 0.49 |
| 127132 | AA721156 | Hs. 190440 | ESTs | 0.49 |
| 129980 | T72661 | Hs. 13969 | ESTs | 0.49 |
| 105323 | AA234112 | Hs. 29075 | ESTs | 0.49 |
| 114439 | AA018937 | Hs. 128629 | ESTs | 0.49 |
| 107632 | AA007242 | Hs. 60179 | EST | 0.49 |
| 130952 | AB002296 | Hs. 21560 | Human mRNA for KIAA0298 gene; complete c | 0.49 |
| 127595 | AA927308 | Hs. 130464 | ESTs | 0.49 |
| 124276 | H83465 | Hs. 221934 | ESTs | 0.49 |
| 125935 | H30721 | Hs. 30172 | ESTs | 0.49 |
| 131275 | U45974 | Hs. 25156 | Human phosphatidylinositol (4;5) bisphos | 0.49 |
| 131196 | C20633 | Hs. 24129 | ESTs | 0.49 |
| 125505 | AI127843 | Hs. 155071 | ESTs | 0.5 |
| 113327 | T71776 | Hs. 12097 | ESTs | 0.5 |
| 104709 | AA017146 | Hs. 34579 | ESTs; Moderately similar to !!!! ALU SUB | 0.5 |
| 115772 | AA423972 | Hs. 8154 | ESTs | 0.5 |
| 118296 | N63150 | Hs. 48723 | ESTs | 0.5 |
| 131453 | C20596 | Hs. 26985 | KIAA0457 protein | 0.5 |
| 104734 | AA019528 | Hs. 32677 | ESTs | 0.5 |
| 119358 | T70550 | Hs. 193651 | ESTs; Weakly similar to alternatively sp | 0.5 |

Pkey: Unique Eos probeset identifier number
ExAccn: Exemplar Accession number, Genbank accession number
UnigeneID: Unigene number
Unigene Title: Unigene gene title

TABLE 19
B survivor vs Mets - Up in B survivor

| Pkey | Ex Accn | UniG_ID | Complete Title | Ratio BS/Met |
|---|---|---|---|---|
| 333601 | | | CH22_FGENES.213_4 | 5.5 |
| 325300 | | | CH.11_hs gi\|5866908 | 4.67 |
| 333642 | | | CH22_FGENES.231_2 | 4.64 |
| 333591 | | | CH22_FGENES.208_4 | 4.46 |
| 332859 | | | CH22_FGENES.27_2 | 4.39 |
| 304013 | AW518573 | Hs. 156110 | Immunoglobulin kappa variable 1D-8 | 4.23 |
| 333791 | | | CH22_FGENES.274_10 | 4.18 |
| 327641 | | | CH.04_hs gi\|5867890 | 4.03 |
| 321172 | H49160 | Hs. 133472 | ESTs | 3.9 |
| 334125 | | | CH22_FGENES.334_4 | 3.88 |
| 333646 | | | CH22_FGENES.234_2 | 3.88 |
| 326554 | | | CH.19_hs gi\|5867308 | 3.84 |
| 333650 | | | CH22_FGENES.238_3 | 3.82 |
| 333647 | | | CH22_FGENES.235_2 | 3.79 |
| 333626 | | | CH22_FGENES.224_2 | 3.68 |
| 314671 | AW236550 | Hs. 131914 | ESTs | 3.68 |
| 310847 | AI420523 | Hs. 161282 | ESTs | 3.67 |
| 333657 | | | CH22_FGENES.241_2 | 3.65 |
| 338522 | | | CH22_EM: AC005500.GENSCAN.395-36 | 3.64 |
| 329464 | | | CH.Y_hs gi\|6456788 | 3.6 |
| 328868 | | | CH.07_hs gi\|6381930 | 3.6 |
| 333637 | | | CH22_FGENES.229_2 | 3.59 |
| 329737 | | | CH.14_p2 gi\|6065779 | 3.5 |
| 317828 | AI791749 | Hs. 128896 | ESTs | 3.44 |
| 330520 | M96995 | Hs. 6289 | growth, factor receptor-bound protein 2 | 3.44 |
| 339271 | | | CH22_BA354I12.GENSCAN.11-2 | 3.44 |
| 314927 | AI735482 | Hs. 159580 | ESTs | 3.42 |
| 334782 | | | CH22_FGENES.432_7 | 3.42 |
| 313138 | AW138842 | Hs. 196669 | ESTs | 3.4 |
| 332650 | H51596 | Hs. 5541 | ATPase; Ca++ transporting; ubiquitous | 3.38 |
| 338648 | | | CH22_EM: AC005500.GENSCAN.460-6 | 3.38 |
| 325677 | | | CH.14_hs gi\|5867017 | 3.34 |
| 312639 | H50648 | Hs. 213221 | ESTs; Weakly similar to !!!! ALU SUBFAMI | 3.33 |
| 326545 | | | CH.19_hs gi\|5867307 | 3.32 |
| 318354 | R44616 | Hs. 138280 | ESTs; Moderately similar to !!!! ALU SUB | 3.3 |
| 308385 | AI625428 | | EST singleton (not in UniGene) with exon | 3.26 |
| 328569 | | | CH.07_hs gi\|6004480 | 3.26 |
| 328582 | | | CH.07_hs gi\|6006033 | 3.24 |
| 310975 | AI492857 | Hs. 170940 | ESTs | 3.24 |
| 336883 | | | CH22_FGENES.322-2 | 3.21 |
| 324425 | AW236939 | Hs. 172154 | ESTs | 3.2 |
| 337870 | | | CH22_EM: AC005500.GENSCAN.48-3 | 3.19 |
| 306624 | AI001043 | | EST singleton (not in UniGene) with exon | 3.17 |
| 319091 | Z45264 | | EST cluster (not in UniGene) | 3.16 |
| 335247 | | | CH22_FGENES.516_8 | 3.12 |
| 324945 | AA088768 | | EST cluster (not in UniGene) | 3.1 |
| 319468 | R06504 | | EST cluster (not in UniGene) | 3.09 |
| 301635 | AI590720 | Hs. 192662 | ESTs; Weakly similar to ZINC FINGER PROT | 3.08 |
| 321215 | AW378128 | Hs. 120243 | ESTs; Weakly similar to CGI-56 protein [ | 3.04 |
| 328507 | | | CH.07_hs gi\|5868473 | 3.03 |
| 330266 | | | CH.05_p2 gi\|6671885 | 3.02 |
| 326249 | | | CH.17_hs gi\|5867263 | 3.01 |
| 325649 | | | CH.14_hs gi\|6588011 | 2.99 |
| 304575 | AA496437 | | EST singleton (not in UniGene) with exon | 2.98 |
| 304559 | AA488050 | | EST singleton (not in UniGene) with exon | 2.97 |
| 338412 | | | CH22_EM: AC005500.GENSCAN.341-25 | 2.96 |
| 308707 | AI769997 | | EST singleton (not in UniGene) with exon | 2.95 |
| 313027 | N34307 | Hs. 184003 | ESTs; Weakly similar to !!!! ALU SUBFAMI | 2.95 |
| 306590 | AI000246 | | EST singleton (not in UniGene) with exon | 2.95 |
| 306183 | AA922622 | | EST singleton (not in UniGene) with exon | 2.94 |
| 308611 | AI735372 | Hs. 203820 | EST; Moderately similar to TRANSLATIONAL | 2.94 |
| 332454 | T63265 | Hs. 11186 | ESTs; Weakly similar to transformation-r | 2.94 |
| 330061 | | | CH.17_p2 gi\|6721261 | 2.94 |
| 317671 | AW138139 | Hs. 244598 | ESTs | 2.93 |
| 338705 | | | CH22_EM: AC005500.GENSCAN.480-4 | 2.93 |
| 333737 | | | CH22_FGENES.261_1 | 2.9 |
| 337756 | | | CH22_EM: AC000097.GENSCAN.109-3 | 2.9 |
| 333572 | | | CH22_FGENES.189_1 | 2.89 |
| 335349 | | | CH22_FGENES.539_2 | 2.89 |
| 328835 | | | CH.07_hs gi\|5868339 | 2.89 |
| 319886 | AA984628 | | EST cluster (not in UniGene) | 2.88 |
| 311247 | AI655313 | Hs. 197692 | ESTs | 2.87 |
| 303887 | R72672 | Hs. 193484 | ESTs; Weakly similar to Similarity with | 2.86 |
| 337564 | | | CH22_C65E1.GENSCAN.1-7 | 2.85 |
| 333225 | | | CH22_FGENES.107_3 | 2.84 |
| 314938 | AA515635 | | EST cluster (not in UniGene) | 2.83 |
| 305803 | AA846052 | | EST singleton (not in UniGene) with exon | 2.83 |
| 305264 | AA679505 | | EST singleton (not in UniGene) with exon | 2.83 |
| 332646 | AA386264 | Hs. 5337 | isocitrate dehydrogenase 2 (NADP+); mito | 2.81 |
| 338508 | | | CH22_EM: AC005500.GENSCAN.391-1 | 2.81 |
| 308097 | AI475411 | | EST singleton (not in UniGene) with exon | 2.81 |
| 301130 | AW194167 | Hs. 149418 | ESTs; Weakly similar to salivary proline | 2.8 |
| 325571 | | | CH.12_hs gi\|6552439 | 2.8 |
| 307054 | AI148181 | Hs. 176835 | EST | 2.8 |
| 337456 | | | CH22_FGENES.777-2 | 2.79 |
| 317870 | AI797066 | Hs. 201995 | ESTs | 2.79 |
| 303171 | AA065003 | Hs. 64179 | hypothetical protein | 2.78 |
| 333717 | | | CH22_FGENES.253_3 | 2.76 |
| 303778 | AW505368 | | EST cluster (not in UniGene) with exon h | 2.76 |
| 304918 | AA602697 | | EST singleton (not in UniGene) with exon | 2.76 |
| 319373 | R00371 | | EST cluster (not in UniGene) | 2.75 |
| 336072 | | | CH22_FGENES.685_4 | 2.74 |
| 306023 | AA897764 | | EST singleton (not in UniGene) with exon | 2.74 |
| 336127 | | | CH22_FGENES.701_15 | 2.74 |
| 337355 | | | CH22_FGENES.728-1 | 2.73 |
| 337885 | | | CH22_EM: AC005500.GENSCAN.54-3 | 2.73 |
| 308506 | AI686791 | Hs. 119598 | ribosomal protein L3 | 2.73 |
| 300629 | AA152119 | Hs. 155101 | ATP synthase; H+ transporting; mitochond | 2.73 |
| 333043 | | | CH22_FGENES.70_4 | 2.72 |
| 327736 | | | CH.05_hs gi\|5867940 | 2.72 |
| 333007 | | | CH22_FGENES.60_4 | 2.72 |
| 321966 | AL122111 | | EST cluster (not in UniGene) | 2.72 |
| 323179 | AW452576 | Hs. 156875 | ESTs | 2.72 |
| 332459 | AA609625 | Hs. 112933 | Homo sapiens Tax interaction protein 40 | 2.71 |
| 326224 | | | CH.17_hs gi\|5867230 | 2.71 |
| 329114 | | | CH.X_hs gi\|5868650 | 2.7 |
| 333577 | | | CH22_FGENES.196_2 | 2.69 |
| 300413 | AW090347 | Hs. 243443 | ESTs | 2.67 |
| 304055 | R07994 | | EST singleton (net in UniGene) with exon | 2.67 |
| 301013 | AI935304 | Hs. 125262 | DKFZP586G1624 protein | 2.67 |
| 337848 | | | CH22_EM: AC005500.GENSCAN.33-1 | 2.66 |
| 327946 | | | CH.06_hs gi\|5868206 | 2.66 |
| 306300 | AA937573 | | EST singleton (not in UniGene) with exon | 2.66 |
| 331071 | R01646 | Hs. 200538 | ESTs | 2.65 |
| 304841 | AA587541 | | EST singleton (not in UniGene) with exon | 2.65 |
| 301321 | AI860987 | Hs. 189097 | ESTs | 2.65 |
| 311280 | AI767957 | Hs. 197737 | ESTs; Weakly similar to Y38A8.1 gene pro | 2.65 |
| 338843 | | | CH22_DJ246D7.GENSCAN.8-1 | 2.64 |
| 335720 | | | CH22_FGENES.599_23 | 2.64 |
| 333670 | | | CH22_FGENES.245_4 | 2.64 |
| 313588 | AI803591 | Hs. 209667 | ESTs | 2.64 |
| 335750 | | | CH22_FGENES.602_4 | 2.63 |
| 333240 | | | CH22_FGENES.111_4 | 2.63 |
| 332721 | R70212 | Hs. 79630 | CD79A antigen (immunoglobulin-associated | 2.62 |
| 338747 | | | CH22_EM: AC005500.GENSCAN.511-1 | 2.62 |
| 303582 | AA377444 | | EST cluster (not in UniGene) with exon h | 2.62 |
| 336898 | | | CH22_FGENES.330-1 | 2.62 |
| 325835 | | | CH.16_hs gi\|6552452 | 2.62 |
| 301660 | F13112 | | EST cluster (not in UniGene) with exon h | 2.61 |
| 335968 | | | CH22_FGENES.652_1 | 2.61 |
| 336705 | | | CH22_FGENES.63-2 | 2.6 |
| 309815 | AW292760 | | EST singleton (not in UniGene) with exon | 2.6 |
| 339220 | | | CH22_FF113D11.GENSCAN.6-15 | 2.6 |
| 308582 | AI709056 | | EST singleton (not in UniGene) with exon | 2.6 |
| 334260 | | | CH22_FGENES.367_8 | 2.6 |
| 309963 | AW449073 | | EST singleton (not in UniGene) with exon | 2.6 |
| 300178 | AI282665 | Hs. 166969 | ESTs | 2.59 |
| 335690 | | | CH22_FGENES.596_5 | 2.59 |
| 308127 | AI492187 | | EST singleton (not in UniGene) with exon | 2.59 |
| 337835 | | | CH22_EM: AC005500.GENSCAN.22-4 | 2.58 |
| 333251 | | | CH22_FGENES.116_3 | 2.58 |
| 330319 | | | CH.08_p2 gi\|5932415 | 2.58 |
| 314490 | AI758114 | Hs. 197032 | ESTs | 2.57 |
| 305934 | AA878815 | Hs. 75442 | albumin | 2.57 |
| 329665 | | | CH.14_p2 gi\|6272129 | 2.57 |
| 328558 | | | CH.07_hs gi\|5868489 | 2.57 |
| 336094 | | | CH22_FGENES.691_3 | 2.57 |
| 307899 | AI380270 | | EST singleton (not in UniGene) with exon | 2.57 |
| 339312 | | | CH22_BA354\|12.GENSCAN.22-10 | 2.57 |
| 336442 | | | CH22_FGENES.827_8 | 2.57 |
| 317894 | R60848 | | EST cluster (not in UniGene) | 2.56 |
| 330435 | HG2689-HT2785 | | Mucin 5b, Tracheobronchial (Gb: X74955) | 2.56 |
| 327304 | | | CH.01_hs gi\|5867494 | 2.56 |
| 308859 | AI830787 | | EST singleton (not in UniGene) with exon | 2.55 |
| 302224 | AI951549 | Hs. 161166 | KIAA1094 protein | 2.55 |
| 304324 | AA137045 | | EST singleton (not in UniGene) with exon | 2.54 |
| 338090 | | | CH22_EM: AC005500.GENSCAN.176-3 | 2.53 |
| 334797 | | | CH22_FGENES.434_5 | 2.52 |
| 303535 | AL043430 | | EST cluster (not in UniGene) with exon h | 2.52 |
| 339037 | | | CH22_DA59H18.GENSCAN.26-5 | 2.52 |
| 327846 | | | CH.05_hs gi\|6531962 | 2.52 |
| 325271 | | | CH.11_hs gi\|5866901 | 2.52 |
| 312385 | R42885 | Hs. 215555 | ESTs | 2.51 |
| 302816 | AI733918 | Hs. 204112 | ESTs; Weakly similar to alternatively sp | 2.51 |
| 316941 | AW449871 | Hs. 124591 | ESTs | 2.5 |
| 300184 | AI285912 | Hs. 254515 | ESTs | 2.5 |
| 333762 | | | CH22_FGENES.270_2 | 2.5 |
| 317028 | AA962623 | Hs. 189144 | ESTs; Weakly similar to RENAL SODIUM-DEP | 2.5 |
| 326266 | | | CH.17_hs gi\|5867264 | 2.49 |
| 326005 | | | CH.16_hs gi\|5867112 | 2.49 |
| 301971 | AJ003125 | Hs. 120330 | a disintegrin-like and metalloprotease ( | 2.48 |
| 326539 | | | CH.19_hs gi\|5867307 | 2.48 |
| 338896 | | | CH22_DJ32I10.GENSCAN.9-4 | 2.48 |
| 306773 | AI040750 | | EST singleton (not in UniGene) with exon | 2.47 |
| 336279 | | | CH22_FGENES.763_3 | 2.47 |
| 321017 | AL050345 | Hs. 227637 | hypothetical protein | 2.47 |
| 306090 | AA908609 | | EST singleton (not in UniGene) with exon | 2.47 |
| 333216 | | | CH22_FGENES.104_8 | 2.46 |
| 338593 | | | CH22_EM: AC005500.GENSCAN.435-2 | 2.46 |
| 333587 | | | CH22_FGENES.205_2 | 2.46 |
| 300396 | AW295466 | Hs. 232051 | ESTs | 2.45 |
| 304693 | AA554263 | | EST singleton (not in UniGene) with exon | 2.45 |
| 338934 | | | CH22_DJ32I10.GENSCAN.18-2 | 2.45 |
| 325751 | | | CH.14_hs gi\|6682474 | 2.45 |
| 334137 | | | CH22_FGENES.337_1 | 2.45 |
| 333581 | | | CH22_FGENES.200_1 | 2.45 |
| 302083 | AI422807 | Hs. 134012 | C1q-related factor | 2.44 |
| 307318 | AI208577 | | EST singleton (not in UniGene) with exon | 2.44 |
| 302181 | AW374284 | Hs. 157732 | Homo sapiens chromosome 19; cosmid R2689 | 2.44 |
| 337425 | | | CH22_FGENES.761-1 | 2.44 |
| 336227 | | | CH22_FGENES.730_2 | 2.44 |
| 314657 | AI015953 | Hs. 125265 | ESTs | 2.44 |
| 338529 | | | CH22_EM: AC005500.GENSCAN.398-10 | 2.44 |
| 333680 | | | CH22_FGENES.247_7 | 2.43 |
| 324834 | AJ003258 | Hs. 250891 | ESTs | 2.43 |
| 305093 | AA642917 | | EST singleton (not in UniGene) with exon | 2.43 |
| 335787 | | | CH22_FGENES.611_3 | 2.43 |
| 311704 | AI655206 | Hs. 121512 | ESTs; Moderately similar to kinesin like | 2.43 |
| 329382 | | | CH.X_hs gi\|5868868 | 2.42 |
| 334785 | | | CH22_FGENES.432_10 | 2.42 |
| 330130 | | | CH.21_p2 gi\|6002196 | 2.42 |
| 327206 | | | CH.01_hs gi\|5867447 | 2.41 |
| 319235 | F11330 | Hs. 177633 | ESTs | 2.41 |
| 334691 | | | CH22_FGENES.420_4 | 2.4 |
| 327610 | | | CH.04_hs gi\|5867868 | 2.4 |
| 327646 | | | CH.04_hs gi\|5867894 | 2.4 |
| 337093 | | | CH22_FGENES.465-18 | 2.4 |
| 335081 | | | CH22_FGENES.488_4 | 2.4 |
| 333576 | | | CH22_FGENES.193_2 | 2.4 |
| 337604 | | | CH22_C20H12.GENSCAN.16-5 | 2.4 |
| 329879 | | | CH.15_p2 gi\|6466518 | 2.4 |
| 328444 | | | CH.07_hs gi\|5868420 | 2.39 |
| 335700 | | | CH22_FGENES.598.1 | 2.39 |
| 331255 | Z41009 | Hs. 21446 | ESTs; Weakly similar to HYPOTHETICAL PRO | 2.39 |
| 327927 | | | CH.06_hs gi\|5868173 | 2.39 |
| 334354 | | | CH22_FGENES.377_1 | 2.39 |
| 308517 | AI689279 | | EST singleton (not in UniGene) with exon | 2.39 |
| 303669 | AW499648 | Hs. 233750 | copine V | 2.39 |
| 333648 | | | CH22_FGENES.237_2 | 2.38 |
| 318318 | AI653893 | Hs. 174463 | ESTs; Weakly similar to alpha3b subunit | 2.38 |
| 338336 | | | CH22_EM: AC005500.GENSCAN.310-8 | 2.38 |
| 304125 | H40976 | | EST singleton (not in UniGene) with exon | 2.38 |
| 304983 | AA617786 | | EST singleton (not in UniGene) with exon | 2.38 |
| 334935 | | | CH22_FGENES.464_3 | 2.38 |
| 314326 | AW170057 | Hs. 133179 | ESTs | 2.38 |
| 330406 | D49490 | Hs. 76901 | for protein disulfide isomerase-related | 2.38 |
| 307646 | AI302236 | | EST singleton (not in UniGene) with exon | 2.38 |
| 338911 | | | CH22_DJ32I10.GENSCAN.11-3 | 2.38 |
| 319952 | T79532 | Hs. 225725 | ESTs; Moderately similar to CGI-101 prot | 2.37 |
| 336878 | | | CH22_FGENES.318-5 | 2.37 |
| 338140 | | | CH22_EM: AC005500.GENSCAN.203-6 | 2.37 |
| 300564 | AI383878 | Hs. 225588 | ESTs | 2.37 |
| 304635 | AA523976 | | EST singleton (not in UniGene) with exon | 2.37 |
| 334091 | | | CH22_FGENES.327_47 | 2.37 |
| 336328 | | | CH22_FGENES.812_7 | 2.37 |
| 325310 | | | CH.11_hs gi\|5866864 | 2.37 |
| 338043 | | | CH22_EM: AC005500.GENSCAN.153-2 | 2.37 |
| 307090 | AI161024 | | EST singleton (not in UniGene) with exon | 2.37 |
| 335768 | | | CH22_FGENES.607_2 | 2.37 |
| 334969 | | | CH22_FGENES.466_2 | 2.37 |
| 333640 | | | CH22_FGENES.230_2 | 2.36 |
| 330002 | | | CH.16_p2 gi\|6623963 | 2.36 |
| 338829 | | | CH22-DJ246D7.GENSCAN.5-12 | 2.36 |
| 323808 | AW250114 | | EST cluster (not in UniGene) | 2.36 |
| 327755 | | | CH.05_hs gi\|5867955 | 2.35 |
| 306426 | AA975039 | | EST singleton (not in UniGene) with exon | 2.35 |
| 336481 | | | CH22_FGENES.830_1 | 2.35 |
| 335163 | | | CH22_FGENES.502_7 | 2.35 |
| 322012 | AL137357 | | EST cluster (not in UniGene) | 2.35 |
| 337345 | | | CH22_FGENES.723-1 | 2.35 |
| 334625 | | | CH22_FGENES.414_3 | 2.35 |
| 320957 | AI878933 | | EST cluster (not in UniGene) | 2.35 |
| 334915 | | | CH22_FGENES.457_4 | 2.35 |
| 336295 | | | CH22_FGENES.787_1 | 2.35 |
| 321556 | N46402 | Hs. 14570 | ESTs | 2.35 |
| 338491 | | | CH22_EM: AC005500.GENSCAN.385-2 | 2.35 |
| 335517 | | | CH22_FGENES.571_34 | 2.34 |
| 330639 | X90872 | Hs. 75854 | SULT1C sulfotransferase | 2.34 |
| 310383 | AI263102 | Hs. 145596 | ESTs | 2.34 |
| 331526 | N49967 | Hs. 46624 | ESTs | 2.34 |
| 334396 | | | CH22_FGENES.381_2 | 2.34 |
| 332993 | | | CH22_FGENES.57_2 | 2.34 |
| 327487 | | | CH.02_hs gi\|5867785 | 2.34 |
| 335920 | | | CH22_FGENES.636_16 | 2.33 |
| 336463 | | | CH22_FGENES.829_22 | 2.33 |
| 319000 | Z44318 | | EST cluster (not in UniGene) | 2.33 |
| 332992 | | | CH22_FGENES.57_1 | 2.33 |
| 332920 | | | CH22_FGENES.37_6 | 2.33 |
| 337590 | | | CH22_C20H12.GENSCAN.6-5 | 2.33 |
| 327059 | | | CH.21_hs gi\|6531965 | 2.33 |
| 334399 | | | CH22_FGENES.382_5 | 2.33 |
| 300982 | AA837754 | | EST cluster (not in UniGene) with, exon h | 2.32 |
| 327430 | | | CH.02_hs gi\|5867754 | 2.32 |
| 326808 | | | CH.20_hs gi\|6682504 | 2.32 |
| 309324 | AW015373 | | EST singleton (not in UniGene) with exon | 2.32 |
| 329779 | | | CH.14_p2 gi\|6002090 | 2.32 |
| 330492 | M25809 | Hs. 64173 | ATPase; H+ transporting; lysosomal (vacu | 2.31 |
| 330080 | | | CH.19_p2 gi\|6015314 | 2.31 |
| 334342 | | | CH22_FGENES.375_20 | 2.31 |
| 336306 | | | CH22_FGENES.793_5 | 2.31 |
| 336400 | | | CH22_FGENES.823_15 | 2.31 |
| 323735 | AA323714 | | EST cluster (not in UniGene) | 2.31 |
| 334496 | | | CH22_FGENES.397_12 | 2.31 |
| 336075 | | | CH22_FGENES.687_1 | 2.31 |
| 335566 | | | CH22_FGENES.580_1 | 2.31 |
| 337657 | | | CH22_EM: AC000097.GENSCAN.32-9 | 2.31 |
| 327816 | | | CH.05_hs gi\|5867968 | 2.3 |
| 308465 | AI672480 | | EST singleton (not in UniGene) with exon | 2.3 |
| 330112 | | | CH.19_p2 gi\|6015238 | 2.3 |
| 304465 | AA421948 | | EST singleton (not in UniGene) with exon | 2.3 |
| 308449 | AI660854 | | EST singleton (not in UniGene) with exon | 2.3 |
| 328171 | | | CH.06_hs gi\|5868071 | 2.3 |
| 328271 | | | CH.06_hs gi\|6552415 | 2.3 |
| 328803 | | | CH.07_hs gi\|6004475 | 2.3 |
| 330063 | | | CH.19_p2 gi\|6165044 | 2.29 |
| 312281 | H11643 | | EST cluster (not in UniGene) | 2.29 |
| 328974 | | | CH.09_hs gi\|5868520 | 2.29 |
| 333859 | | | CH22_FGENES.290_18 | 2.29 |
| 326253 | | | CH.17_hs gi\|5867263 | 2.29 |
| 325703 | | | CH.14_hs gi\|5867028 | 2.29 |
| 338925 | | | CH22_DJ32I10.GENSCAN.14-3 | 2.29 |
| 328552 | | | CH.07_hs gi\|5868489 | 2.29 |
| 337244 | | | CH22_FGENES.646-8 | 2.29 |
| 314770 | AI732722 | Hs. 187694 | ESTs | 2.29 |
| 324560 | AW502208 | | EST cluster (not in UniGene) | 2.29 |
| 310603 | AW376860 | Hs. 156398 | ESTs | 2.29 |
| 337363 | | | CH22_FGENES.733-2 | 2.29 |
| 308015 | AI440174 | Hs. 228907 | EST; Weakly similar to GUANINE NUCLEOTID | 2.28 |
| 309206 | AI961962 | | EST singleton (not in UniGene) with exon | 2.28 |
| 337455 | | | CH22_FGENES.777-1 | 2.28 |
| 327605 | | | CH.03_hs gi\|6004463 | 2.28 |
| 301611 | W22172 | Hs. 59038 | ESTs | 2.28 |
| 317222 | AI206964 | Hs. 130051 | ESTs | 2.28 |
| 338278 | | | CH22_EM: AC005500.GENSCAN.290-3 | 2.28 |
| 337291 | | | CH22_FGENES.673-2 | 2.27 |
| 337913 | | | CH22_EM: AC005500.GENSCAN.59-10 | 2.27 |
| 306406 | AA971973 | | EST singleton (not in UniGene) with exon | 2.27 |
| 332947 | | | CH22_FGENES.47_10 | 2.27 |
| 321763 | W01148 | | EST cluster (not in UniGene) | 2.27 |
| 304424 | AA293494 | | EST singleton (not in UniGene) with exon | 2.27 |
| 303782 | T64737 | | EST cluster (not in UniGene) with exon h | 2.27 |
| 326943 | | | CH.21_hs gi\|6004446 | 2.27 |
| 324977 | R14439 | Hs. 209194 | ESTs | 2.27 |
| 325480 | | | CH.12_hs gi\|5866957 | 2.27 |
| 327743 | | | CH.05_hs gi\|5867944 | 2.27 |
| 333221 | | | CH22_FGENES.105_1 | 2.26 |
| 336498 | | | CH22_FGENES.833_3 | 2.26 |
| 321583 | H84421 | | EST cluster (not in UniGene) | 2.26 |
| 334191 | | | CH22_FGENES.352_6 | 2.26 |
| 327089 | | | CH.21_hs gi\|6531965 | 2.26 |
| 310001 | F18939 | Hs. 153827 | ESTs | 2.26 |
| 304056 | R08577 | | EST singleton (not in UniGene) with exon | 2.25 |
| 324700 | AW504745 | Hs. 103913 | ESTs; Moderately similar to !!!! ALU SUB | 2.25 |
| 330637 | X86371 | Hs. 95659 | lethal giant larvae (Drosophila) homolog | 2.25 |
| 307642 | AI302103 | | EST singleton (not in UniGene) with exon | 2.25 |
| 336985 | | | CH22_FGENES.402-6 | 2.25 |
| 334425 | | | CH22_FGENES.384_13 | 2.25 |
| 321216 | AI078042 | Hs. 126691 | ESTs | 2.25 |
| 315785 | AW205946 | Hs. 150319 | ESTs | 2.25 |
| 305809 | AA853998 | Hs. 124580 | EST | 2.25 |
| 331334 | AA284858 | Hs. 89134 | ESTs | 2.25 |
| 317131 | AI991125 | Hs. 189109 | ESTs | 2.25 |
| 334216 | | | CH22_FGENES.358_1 | 2.24 |
| 330330 | | | CH.08_p2 gi\|5670267 | 2.24 |
| 326923 | | | CH.21_hs gi\|6456782 | 2.24 |
| 333774 | | | CH22_FGENES.272_5 | 2.24 |
| 324311 | AA443061 | Hs. 202520 | ESTs | 2.24 |
| 338551 | | | CH22_EM: AC005500.GENSCAN.413-2 | 2.24 |
| 306716 | AI024916 | Hs. 251354 | ESTs | 2.24 |
| 337689 | | | CH22_EM: AC000097.GENSCAN.77-5 | 2.24 |
| 300079 | AI192520 | Hs. 147178 | EST | 2.23 |
| 334617 | | | CH22_FGENES.411_16 | 2.23 |
| 336890 | | | CH22_FGENES.326-10 | 2.23 |
| 334495 | | | CH22_FGENES.397_10 | 2.23 |
| 327301 | | | CH.01_hs gi\|5867493 | 2.23 |
| 337856 | | | CH22_EM: AC005500.GENSCAN.41-3 | 2.23 |
| 307072 | AI150424 | Hs. 146817 | EST | 2.23 |
| 330515 | M85247 | | H. sapiens dopamine D1A receptor gene, co | 2.22 |
| 325943 | | | CH.16_hs gi\|5867138 | 2.22 |
| 338947 | | | CH22_DJ32I10.GENSCAN.21-4 | 2.22 |
| 317465 | AW197361 | Hs. 131360 | ESTs | 2.22 |
| 332458 | M33493 | Hs. 184504 | tryptase; alpha | 2.22 |
| 333195 | | | CH22_FGENES.98_17 | 2.22 |
| 304837 | AA587139 | | EST singleton (not in UniGene) with exon | 2.22 |
| 307602 | AI288843 | Hs. 231239 | EST | 2.22 |
| 337078 | | | CH22_FGENES.457-1 | 2.22 |
| 335862 | | | CH22_FGENES.629_7 | 2.22 |
| 301979 | L28168 | Hs. 121495 | potassium voltage-gated channel; lsk-rel | 2.22 |
| 335668 | | | CH22_FGENES.590_19 | 2.22 |
| 305068 | AA639618 | | EST singleton (not in UniGene) with exon | 2.21 |
| 329034 | | | CH.X_hs gi\|5868561 | 2.21 |
| 318403 | AI131241 | Hs. 143234 | ESTs | 2.21 |
| 328058 | | | CH.06_hs gi\|5902482 | 2.21 |
| 335513 | | | CH22_FGENES.571_28 | 2.21 |
| 330803 | AA004699 | Hs. 150580 | putative translation initiation factor | 2.21 |
| 331427 | H54764 | Hs. 237339 | EST | 2.21 |
| 338973 | | | CH22_DJ32I10.GENSCAN.27-6 | 2.2 |
| 336723 | | | CH22_FGENES.85-3 | 2.2 |
| 327290 | | | CH.01_hs gi\|5867483 | 2.2 |
| 337240 | | | CH22_FGENES.644-1 | 2.2 |
| 306201 | AA926818 | | EST singleton (not in UniGene) with exon | 2.2 |
| 303659 | AA868464 | Hs. 126263 | ESTs; Highly similar to FIBRILLARIN [H.s | 2.2 |
| 334517 | | | CH22_FGENES.399_7 | 2.2 |
| 334189 | | | CH22_FGENES.352_4 | 2.2 |
| 335199 | | | CH22_FGENES.508_8 | 2.2 |
| 333705 | | | CH22_FGENES.250_19 | 2.2 |
| 305794 | AA845324 | | EST singleton (not in UniGene) with exon | 2.2 |
| 303273 | AA316069 | | EST cluster (not in UniGene) with exon h | 2.2 |
| 313384 | W85694 | Hs. 118335 | ESTs | 2.2 |
| 329158 | | | CH.X_hs gi\|5868687 | 2.2 |
| 337551 | | | CH22_FGENES.847-8 | 2.2 |
| 328792 | | | CH.07_hs gi\|5868309 | 2.2 |
| 303737 | AW502711 | | EST cluster (not in UniGene) with exon h | 2.19 |
| 324529 | AW502466 | | EST cluster (not in UniGene) | 2.19 |
| 323103 | Z45529 | Hs. 92030 | ESTs | 2.19 |
| 333773 | | | CH22_FGENES.272_4 | 2.19 |
| 337906 | | | CH22_EM: AC005500.GENSCAN.56-19 | 2.19 |
| 327129 | | | CH.21_hs gi\|6531976 | 2.19 |
| 305710 | AA826544 | | EST singleton (not in UniGene) with exon | 2.19 |
| 335595 | | | CH22_FGENES.581_34 | 2.19 |
| 323646 | AA310926 | Hs. 154412 | ESTs | 2.19 |
| 328368 | | | CH.07_hs gi\|5868388 | 2.19 |
| 325802 | | | CH.14_hs gi\|6552451 | 2.19 |
| 337167 | | | CH22_FGENES.562-27 | 2.19 |
| 305059 | AA635756 | | EST singleton (not in UniGene) with exon | 2.18 |
| 321445 | AW245524 | Hs. 121590 | ESTs; Weakly similar to ZINC FINGER PROT | 2.18 |
| 332790 | | | CH22_FGENES.2_4 | 2.18 |
| 336750 | | | CH22_FGENES.128-4 | 2.18 |
| 310999 | AI520706 | Hs. 171012 | ESTs | 2.18 |
| 329798 | | | CH.14_p2 gi\|6523160 | 2.18 |
| 327012 | | | CH.21_hs gi\|5867664 | 2.18 |
| 304599 | AA506638 | | EST singleton (not in UniGene) with exon | 2.18 |
| 335351 | | | CH22_FGENES.539_4 | 2.18 |
| 310661 | AI354717 | Hs. 223908 | ESTs | 2.18 |
| 332791 | | | CH22_FGENES.3_1 | 2.17 |
| 333022 | | | CH22_FGENES.65_1 | 2.17 |
| 310502 | AI458973 | Hs. 170422 | ESTs | 2.17 |
| 324963 | AA853440 | | EST cluster (not in UniGene) | 2.17 |
| 325275 | | | CH.11_hs gi\|5866902 | 2.17 |
| 328338 | | | CH.07_hs gi\|5868377 | 2.17 |
| 333063 | | | CH22_FGENES.75_6 | 2.17 |
| 308895 | AI858423 | | EST singleton (not in UniGene) with exon | 2.17 |
| 338685 | | | CH22_EM: AC005500.GENSCAN.472-4 | 2.16 |
| 325655 | | | CH.14_hs gi\|5867007 | 2.16 |
| 332420 | H49570 | Hs. 108074 | ESTs; Weakly similar to CEREBELLIN 1 PRE | 2.16 |
| 337216 | | | CH22_FGENES.613-10 | 2.16 |
| 335660 | | | CH22_FGENES.590_11 | 2.16 |
| 337145 | | | CH22_FGENES.542-2 | 2.16 |
| 335753 | | | CH22_FGENES.604_2 | 2.16 |
| 301766 | R02224 | | EST cluster (not in UniGene) with exon h | 2.16 |
| 303442 | AI953998 | Hs. 152510 | ESTs; Weakly similar to L-SERINE DEHYDRA | 2.16 |
| 311009 | AI949701 | Hs. 210589 | ESTs | 2.16 |
| 307093 | AI167606 | | EST singleton (not in UniGene) with exon | 2.16 |
| 300262 | AI874402 | Hs. 170810 | ESTs | 2.16 |
| 337989 | | | CH22_EM: AC005500.GENSCAN.112-7 | 2.16 |
| 326263 | | | CH.17_hs gi\|5867264 | 2.16 |
| 319402 | W21298 | | EST cluster (not in UniGene) | 2.16 |
| 321010 | Y17456 | Hs. 227150 | Homo sapiens LSFR2 gene; last exon | 2.16 |
| 301706 | AI929150 | Hs. 241496 | ESTs | 2.16 |
| 307412 | AI241753 | Hs. 241507 | ribosomal protein S6 | 2.16 |
| 335662 | | | CH22_FGENES.590_13 | 2.15 |
| 332480 | AA092932 | Hs. 12570 | tubulin-specific chaperone d | 2.15 |
| 329273 | | | CH.X_hs gi\|5868762 | 2.15 |
| 339383 | | | CH22_BA232E17.GENSCAN.3-20 | 2.15 |
| 332795 | | | CH22_FGENES.5_1 | 2.15 |
| 335227 | | | CH22_FGENES.513_13 | 2.15 |
| 326925 | | | CH.21_hs gi\|6456782 | 2.15 |
| 332403 | AA424199 | Hs. 106529 | ESTs; Highly similar to CGI-65 protein [ | 2.15 |
| 317786 | AI859605 | Hs. 155686 | ESTs | 2.15 |
| 326582 | | | CH.19_hs gi\|5867318 | 2.15 |
| 336494 | | | CH22_FGENES.832_11 | 2.15 |
| 329656 | | | CH.14_p2 gi\|6448516 | 2.15 |
| 307581 | AI284415 | | EST singleton (not in UniGene) with exon | 2.15 |
| 335670 | | | CH22_FGENES.591_2 | 2.14 |
| 332452 | AA040369 | Hs. 11170 | SYT interacting protein | 2.14 |
| 309387 | AW079943 | Hs. 156110 | Immunoglobulin kappa variable 1D-8 | 2.14 |
| 308427 | AI652677 | Hs. 195055 | EST | 2.14 |
| 322027 | NM_004551 | | EST cluster (not in UniGene) | 2.14 |
| 301693 | Z45023 | | EST cluster (not in UniGene) with exon h | 2.14 |
| 334308 | | | CH22_FGENES.373_11 | 2.14 |
| 301131 | AW134518 | Hs. 131807 | ESTs | 2.13 |
| 338495 | | | CH22_EM: AC005500.GENSCAN.387-1 | 2.13 |
| 329600 | | | CH.10_p2 gi\|3962481 | 2.13 |
| 307980 | AI431696 | | EST singleton (not in UniGene) with exon | 2.13 |
| 337260 | | | CH22_FGENES.652-15 | 2.13 |
| 304655 | AA527887 | | EST singleton (not in UniGene) with exon | 2.13 |
| 303141 | AF195951 | | EST cluster (not in UniGene) with exon h | 2.13 |
| 327957 | | | CH.06_hs gi\|5868210 | 2.13 |
| 334317 | | | CH22_FGENES.374_1 | 2.13 |
| 302870 | AF011407 | | EST cluster (not in UniGene) with exon h | 2.13 |
| 333806 | | | CH22_FGENES.278_2 | 2.13 |
| 329947 | | | CH.16_p2 gi\|5540101 | 2.13 |
| 309602 | AW182523 | | EST singleton (not in UniGene) with exon | 2.13 |
| 322790 | AI700273 | Hs. 122162 | ESTs; Weakly similar to KIAA0557 protein | 2.13 |
| 337706 | | | CH22_EM: AC000097.GENSCAN.87-11 | 2.13 |
| 306894 | AI092731 | | EST singleton (not in UniGene) with exon | 2.13 |
| 325530 | | | CH.12_hs gi\|6525289 | 2.12 |
| 321087 | AL110227 | Hs. 241533 | Homo sapiens mRNA; cDNA DKFZp434J194 (fr | 2.12 |
| 309853 | AW298169 | Hs. 57553 | tousled-like kinase 2 | 2.12 |
| 326822 | | | CH.20_hs gi\|6117831 | 2.12 |
| 328776 | | | CH.07_hs gi\|5868309 | 2.12 |
| 335112 | | | CH22_FGENES.494_20 | 2.12 |
| 334564 | | | CH22_FGENES.405_4 | 2.12 |
| 333455 | | | CH22_FGENES.157_4 | 2.12 |
| 317395 | R55044 | Hs. 124130 | ESTs | 2.12 |
| 334221 | | | CH22_FGENES.360_1 | 2.12 |
| 331374 | AA442134 | Hs. 70573 | ESTs; Weakly similar to HINT PROTEIN [H. | 2.12 |
| 304473 | AA428343 | Hs. 140 | immunoglobulin gamma 3 (Gm marker) | 2.12 |
| 328907 | | | CH.08_hs gi\|5868493 | 2.12 |
| 319448 | R05539 | Hs. 108738 | ESTs | 2.12 |
| 333676 | | | CH22_FGENES.247_3 | 2.12 |
| 324767 | AA630931 | Hs. 34348 | Homo sapiens mRNA; cDNA DKFZp434P0235 (f | 2.12 |
| 318585 | Z43405 | | EST cluster (not in UniGene) | 2.12 |
| 331732 | AA251192 | Hs. 177708 | ESTs | 2.12 |
| 329553 | | | CH.10_p2 gi\|3962492 | 2.12 |
| 336910 | | | CH22_FGENES.343-6 | 2.12 |
| 326959 | | | CH.21_hs gi\|6469836 | 2.12 |
| 305417 | AA725228 | | EST singleton (not in UniGene) with exon | 2.11 |
| 301573 | AI150328 | Hs. 226402 | ESTs; Weakly similar to mitochondrial ci | 2.11 |
| 326935 | | | CH.21_hs gi\|6004446 | 2.11 |
| 335176 | | | CH22_FGENES.504_6 | 2.11 |
| 337210 | | | CH22_FGENES.603-5 | 2.11 |
| 311284 | AW027025 | Hs. 239262 | ESTs | 2.11 |
| 330240 | | | CH.05_p2 gi\|6671858 | 2.11 |
| 327463 | | | CH.02_hs gi\|6004455 | 2.11 |
| 332938 | | | CH22_FGENES.41_3 | 2.11 |
| 332785 | | | CH22_FGENES.1_1 | 2.11 |
| 301035 | AI358105 | Hs. 123164 | ESTs | 2.1 |
| 305712 | AA826701 | | EST singleton (not in UniGene) with exon | 2.1 |
| 318651 | AW003150 | Hs. 240165 | ESTs | 2.1 |
| 302753 | M74299 | | EST cluster (not in UniGene) with exon h | 2.1 |
| 334635 | | | CH22_FGENES.417_2 | 2.1 |
| 319447 | AA456745 | | EST cluster (not in UniGene) | 2.1 |
| 301204 | AW008544 | Hs. 239994 | ESTs | 2.1 |
| 333950 | | | CH22_FGENES.303_6 | 2.1 |
| 325947 | | | CH.16_hs gi\|5867138 | 2.1 |
| 337683 | | | CH22_EM: AC000097.GENSCAN.76-1 | 2.1 |
| 328962 | | | CH.08_hs gi\|6456775 | 2.1 |
| 336655 | | | CH22_FGENES.34-3 | 2.1 |
| 336596 | | | CH22_FGENES.163_2 | 2.1 |
| 330486 | M13755 | Hs. 833 | interferon-stimulated protein; 15 kDa | 2.1 |
| 314356 | AA531607 | Hs. 125143 | ESTs | 2.09 |
| 314976 | AA524725 | Hs. 162108 | ESTs | 2.09 |
| 336650 | | | CH22_FGENES.29-6 | 2.09 |
| 339026 | | | CH22_DA59H18.GENSCAN.22-6 | 2.09 |
| 302395 | AW297357 | Hs. 114606 | ESTs | 2.09 |
| 323280 | AI910263 | | EST cluster (not in UniGene) | 2.09 |
| 338857 | | | CH22_DJ32I10.GENSCAN.1-1 | 2.09 |
| 335374 | | | CH22_FGENES.543_12 | 2.09 |
| 308766 | AI808510 | | EST singleton (not in UniGene) with exon | 2.09 |
| 331027 | N48584 | Hs. 6168 | KIAA0703 gene product | 2.09 |
| 337853 | | | CH22_EM: AC005500.GENSCAN.37-1 | 2.09 |
| 302498 | NM_002991 | | EST cluster (not in UniGene) with exon h | 2.09 |
| 312607 | AI337440 | Hs. 169375 | ESTs | 2.09 |
| 314309 | Z44049 | Hs. 184352 | ESTs; Weakly similar to cDNA EST EMBL: D3 | 2.09 |
| 311695 | AI142078 | Hs. 135562 | ESTs | 2.09 |
| 333280 | | | CH22_FGENES.126_2 | 2.09 |
| 333518 | | | CH22_FGENES.173_3 | 2.09 |
| 337199 | | | CH22_FGENES.583-11 | 2.09 |
| 337819 | | | CH22_EM: AC005500.GENSCAN.13-9 | 2.08 |
| 300546 | AA214450 | Hs. 250913 | ESTs | 2.08 |
| 322577 | AA354452 | Hs. 59075 | ESTs; Weakly similar to WD40 protein Cia | 2.08 |
| 336028 | | | CH22_FGENES.672_1 | 2.08 |
| 300238 | AI394673 | Hs. 254030 | ESTs | 2.08 |
| 307429 | AI243573 | | EST singleton (not in UniGene) with exon | 2.08 |
| 326444 | | | CH.19_hs gi\|5867385 | 2.08 |
| 310641 | AI345597 | Hs. 254727 | ESTs | 2.08 |
| 337633 | | | CH22_C20H12.GENSCAN.32-1 | 2.08 |
| 336008 | | | CH22_FGENES.668_6 | 2.08 |
| 339030 | | | CH22_DA59H18.GENSCAN.24-1 | 2.08 |
| 333952 | | | CH22_FGENES.303_8 | 2.08 |
| 329149 | | | CH.X_hs gi\|5868685 | 2.08 |
| 335192 | | | CH22_FGENES.507_7 | 2.08 |
| 308225 | AI557713 | Hs. 177592 | ribosomal protein; large; P1 | 2.08 |
| 330519 | M94172 | Hs. 69949 | calcium channel; voltage-dependent; L ty | 2.08 |
| 331809 | AA402482 | Hs. 97312 | ESTs | 2.07 |
| 324837 | AJ003669 | Hs. 246171 | ESTs | 2.07 |
| 332608 | D00749 | Hs. 36972 | CD7 antigen (p41) | 2.07 |
| 327291 | | | CH.01_hs gi\|5867483 | 2.07 |
| 315936 | AW069807 | Hs. 247094 | ESTs; Moderately similar to !!!! ALU SUB | 2.07 |
| 317917 | AI143593 | Hs. 129419 | ESTs | 2.07 |
| 328674 | | | CH.07_hs gi\|5868254 | 2.07 |
| 338654 | | | CH22_EM: AC005500.GENSCAN.460-55 | 2.07 |
| 320828 | AJ012590 | Hs. 194728 | hexose-6-phosphate dehydrogenase (glucos | 2.07 |
| 337896 | | | CH22_EM: AC005500.GENSCAN.56-3 | 2.07 |
| 335310 | | | CH22_FGENES.532_3 | 2.07 |
| 300076 | AW074835 | Hs. 145223 | ESTs | 2.07 |
| 303588 | AL046182 | | EST cluster (not in UniGene) with exon h | 2.07 |
| 328848 | | | CH.07_hs gi\|6381921 | 2.07 |
| 318723 | C18060 | | EST cluster (not in UniGene) | 2.07 |
| 335352 | | | CH22_FGENES.539_5 | 2.07 |
| 339316 | | | CH22_BA354I12.GENSCAN.22-15 | 2.06 |
| 335873 | | | CH22_FGENES.631_1 | 2.06 |
| 335261 | | | CH22_FGENES.520_2 | 2.06 |
| 322032 | AL079807 | | EST cluster (not in UniGene) | 2.06 |
| 308771 | AI809301 | | EST singleton (not in UniGene) with exon | 2.06 |
| 310024 | AI252661 | Hs. 145224 | ESTs | 2.06 |
| 320555 | R36212 | Hs. 235534 | ESTs | 2.06 |
| 319314 | T74062 | | EST cluster (not in UniGene) | 2.06 |
| 334642 | | | CH22_FGENES.417_9 | 2.06 |
| 335767 | | | CH22_FGENES.607_1 | 2.06 |
| 336159 | | | CH22_FGENES.707_3 | 2.06 |
| 336358 | | | CH22_FGENES.818_1 | 2.06 |
| 334687 | | | CH22_FGENES.419_12 | 2.06 |
| 339389 | | | CH22_BA232E17.GENSCAN.4-7 | 2.06 |
| 335898 | | | CH22_FGENES.635_6 | 2.06 |
| 328847 | | | CH.07_hs gi\|6381920 | 2.06 |
| 313431 | W91884 | | EST cluster (not in UniGene) | 2.06 |
| 313270 | AI374993 | Hs. 159611 | ESTs | 2.06 |
| 339211 | | | CH22_FF113D11.GENSCAN.6-6 | 2.06 |
| 333860 | | | CH22_FGENES.290_19 | 2.06 |
| 308952 | AI868157 | Hs. 224226 | EST | 2.06 |
| 305471 | AA743947 | | EST singleton (not in UniGene) with exon | 2.06 |
| 300619 | AA991438 | Hs. 233293 | ESTs | 2.06 |
| 302962 | AI693349 | Hs. 228981 | EST | 2.06 |
| 332446 | AA112799 | Hs. 238756 | ESTs; Weakly similar to unknown [H.sapie | 2.06 |
| 334972 | | | CH22_FGENES.468_2 | 2.05 |
| 330196 | | | CH.05_p2 gi\|6165140 | 2.05 |
| 304754 | AA579795 | | EST singleton (not in UniGene) with exon | 2.05 |
| 309726 | AW248521 | Hs. 195188 | glyceraldehyde-3-phosphate dehydrogenase | 2.05 |
| 333939 | | | CH22_FGENES.301_5 | 2.05 |
| 304836 | AA587008 | | EST singleton (not in UniGene) with exon | 2.05 |
| 302087 | AA324163 | | EST cluster (not in UniGene) with exon h | 2.05 |
| 308424 | AI650714 | | EST singleton (not in UniGene) with exon | 2.05 |
| 304347 | AA176914 | | EST singleton (not in UniGene) with exon | 2.05 |
| 333141 | | | CH22_FGENES.85._1 | 2.05 |
| 310573 | AW292180 | Hs. 156142 | ESTs | 2.05 |
| 337565 | | | CH22_C65E1.GENSCAN.1-11 | 2.05 |
| 304295 | AA084082 | | EST singleton (not in UniGene) with exon | 2.05 |
| 326624 | | | CH.20_hs gi\|5867553 | 2.05 |
| 326443 | | | CH.19_hs gi\|5867385 | 2.04 |
| 339012 | | | CH22_DA59H18.GENSCAN.19-2 | 2.04 |
| 337384 | | | CH22_FGENES.745-1 | 2.04 |
| 332326 | T79623 | Hs. 111787 | ESTs | 2.04 |
| 303706 | AW501525 | | EST cluster (not in UniGene) with exon h | 2.04 |
| 336046 | | | CH22_FGENES.679_8 | 2.04 |
| 301770 | R05887 | | EST cluster (not in UniGene) with exon h | 2.04 |
| 326726 | | | CH.20_hs gi\|5867597 | 2.04 |
| 330485 | M11186 | Hs. 113216 | oxytocin; prepro-(neurophysin I) | 2.04 |
| 332956 | | | CH22_FGENES.48_13 | 2.04 |
| 300021 | M97935 | | AFFX control: STAT1 | 2.04 |
| 306872 | AI086920 | | EST singleton (not in UniGene) with exon | 2.03 |
| 302744 | L03151 | | EST cluster (not in UniGene) with exon h | 2.03 |
| 338507 | | | CH22_EM: AC005500.GENSCAN.390-11 | 2.03 |
| 334020 | | | CH22_FGENES.317_1 | 2.03 |
| 333870 | | | CH22_FGENES.291_3 | 2.03 |
| 330552 | U40223 | Hs. 248157 | pyrimidinergic receptor P2Y; G-protein c | 2.03 |
| 335486 | | | CH22_FGENES.570_18 | 2.03 |
| 339374 | | | CH22_BA232E17.GENSCAN.2-5 | 2.03 |
| 328384 | | | CH.07_hs gi\|5868392 | 2.03 |
| 334690 | | | CH22_FGENES.420_3 | 2.03 |
| 310318 | AI733942 | Hs. 145338 | ESTs | 2.03 |
| 325893 | | | CH.16_hs gi\|5867088 | 2.03 |
| 331373 | AA435513 | Hs. 178170 | ESTs; Weakly similar to DUAL SPECIFICITY | 2.03 |
| 329784 | | | CH.14_p2 gi\|5912597 | 2.03 |
| 335087 | | | CH22_FGENES.488_11 | 2.03 |
| 310582 | AI336563 | Hs. 254585 | ESTs | 2.03 |
| 332611 | R06751 | Hs. 1600 | chaperonin containing TCP1; subunit 5 (e | 2.03 |
| 339258 | | | CH22_BA354I12.GENSCAN.8-3 | 2.03 |
| 336851 | | | CH22_FGENES.274-1 | 2.03 |
| 305596 | AA780664 | Hs. 8734 | ESTs; Moderately similar to !!!! ALU CLA | 2.03 |
| 330364 | | | CH.X_p2 gi\|3126882 | 2.03 |
| 302940 | AL137619 | | EST cluster (not in UniGene) with exon h | 2.03 |
| 317349 | AA923657 | Hs. 126359 | ESTs; Weakly similar to !!!! ALU SUBFAMI | 2.03 |
| 309869 | AW300314 | | EST singleton (not in UniGene) with exon | 2.03 |
| 333422 | | | CH22_FGENES.147_2 | 2.03 |
| 325233 | | | CH.10_hs gi\|6381943 | 2.03 |
| 330586 | U77968 | Hs. 79564 | neuronal PAS domain protein 1 | 2.03 |
| 336725 | | | CH22_FGENES.88-1 | 2.02 |
| 334157 | | | CH22_FGENES.340_7 | 2.02 |
| 303357 | AW006352 | Hs. 159643 | ESTs; Weakly similar to MLD [H. sapiens] | 2.02 |
| 328533 | | | CH.07_hs gi\|5868482 | 2.02 |
| 309210 | AI962817 | | EST singleton (not in UniGene) with exon | 2.02 |
| 327412 | | | CH.02_hs gi\|5867750 | 2.02 |
| 333172 | | | CH22_FGENES.94_7 | 2.02 |
| 334869 | | | CH22_FGENES.447_3 | 2.02 |
| 301047 | AA971465 | Hs. 116136 | ESTs | 2.02 |
| 329394 | | | CH.X_hs gi\|6478817 | 2.02 |
| 301736 | F12128 | | EST cluster (not in UniGene) with exon h | 2.02 |
| 335591 | | | CH22_FGENES.581_30 | 2.02 |
| 338234 | | | CH22_EM: AC005500.GENSCAN.260-7 | 2.02 |
| 334433 | | | CH22_FGENES.385_8 | 2.02 |
| 334904 | | | CH22_FGENES.452_18 | 2.02 |
| 318443 | AI939323 | Hs. 157714 | ESTs; Weakly similar to NEUR ACETYLCHOLI | 2.02 |
| 300151 | AI243445 | Hs. 189654 | ESTs | 2.01 |
| 310348 | AI478563 | Hs. 145519 | ESTs | 2.01 |
| 310898 | AI439868 | Hs. 165742 | ESTs | 2.01 |
| 332860 | | | CH22_FGENES.27_3 | 2.01 |
| 301699 | AI879117 | | EST cluster (not in UniGene) with exon h | 2.01 |
| 332554 | W96450 | Hs. 23111 | phenylalanine-tRNA synthetase-like | 2.01 |
| 327994 | | | CH.06_hs gi\|5868218 | 2.01 |
| 315613 | AW137420 | Hs. 192311 | ESTs | 2.01 |
| 335356 | | | CH22_FGENES.541_3 | 2.01 |
| 334028 | | | CH22_FGENES.318_7 | 2.01 |
| 335277 | | | CH22_FGENES.523_3 | 2.01 |
| 308657 | AI749855 | Hs. 236497 | EST; Weakly similar to GLANDULAR KALLIKR | 2.01 |
| 305913 | AA876109 | | EST singleton (not in UniGene) with exon | 2.01 |
| 323681 | AW247730 | Hs. 102548 | glucocorticoid receptor DNA binding fact | 2.01 |
| 333533 | | | CH22_FGENES.175_20 | 2.01 |
| 328753 | | | CH.07_hs gi\|5868298 | 2.01 |
| 302397 | L01694 | Hs. 211523 | guanine nucleotide binding protein (G pr | 2.01 |
| 304643 | AA526588 | | EST singleton (not in UniGene) with exon | 2.01 |
| 333065 | | | CH22_FGENES.75_8 | 2.01 |
| 316192 | AA904441 | Hs. 221286 | ESTs | 2 |
| 302533 | L36149 | Hs. 248116 | chemokine (C motif) XC receptor 1 | 2 |
| 312988 | AA813689 | Hs. 123436 | ESTs | 2 |
| 333612 | | | CH22_FGENES.217_7 | 2 |
| 333615 | | | CH22_FGENES.217_10 | 2 |
| 316085 | AI027959 | Hs. 132300 | ESTs | 2 |
| 337936 | | | CH22_EM: AC005500.GENSCAN.85-7 | 2 |
| 330972 | H18467 | Hs. 118983 | ESTs; Weakly similar to diaphanous 1 [H. | 2 |

Pkey: Unique Eos probeset identifier number
ExAccn: Exemplar Accession number, Genbank accession number
UnigeneID: Unigene number
Unigene Title: Unigene gene title

TABLE 20
B survivor vs Mets - Up in Mets

| Pkey | Ex Accn | UniG_ID | Complete Title | Ratio BS/Met |
|---|---|---|---|---|
| 316625 | AA780307 | Hs. 122156 | ESTs | 0.28 |
| 316076 | AW297895 | Hs. 116424 | ESTs | 0.3 |
| 315943 | AA699756 | Hs. 117335 | ESTs | 0.38 |
| 317198 | AI810384 | Hs. 128025 | ESTs | 0.38 |
| 320082 | AA487678 | Hs. 189738 | ESTs | 0.39 |
| 313510 | AI147291 | Hs. 154006 | ESTs | 0.39 |
| 323683 | AI380045 | Hs. 225033 | ESTs | 0.39 |
| 318558 | AW402677 | Hs. 90372 | ESTs | 0.4 |
| 310264 | AI915771 | Hs. 148867 | ESTs | 0.4 |
| 314945 | AW276866 | Hs. 192715 | ESTs | 0.41 |
| 313403 | W86995 | Hs. 113157 | ESTs | 0.42 |
| 321505 | H73183 | Hs. 129885 | ESTs | 0.43 |
| 312171 | AW444619 | Hs. 138211 | ESTs | 0.43 |
| 324585 | AI823969 | Hs. 132678 | ESTs | 0.44 |
| 316695 | AA809844 | | EST cluster | 0.44 |
| | | | (not in UniGene) | |
| 319818 | AA825819 | Hs. 136952 | ESTs | 0.44 |
| 337522 | | | CH22_{—} | 0.45 |
| | | | FGENES.819-1 | |
| 324714 | AA574312 | Hs. 245737 | ESTs | 0.45 |
| 315060 | AA551104 | Hs. 189048 | ESTs | 0.46 |
| 300548 | AI026836 | Hs. 114689 | ESTs | 0.47 |
| 304483 | AA431441 | | EST singleton | 0.47 |
| | | | (not in UniGene) | |
| | | | with exon | |
| 313096 | AI422367 | Hs. 163533 | ESTs | 0.47 |
| 306501 | AA987294 | | EST singleton | 0.47 |
| | | | (not in UniGene) | |
| | | | with exon | |
| 329086 | | | CH.X_hs | 0.47 |
| | | | gi\|5868604 | |
| 320176 | AA167566 | Hs. 133325 | ESTs | 0.47 |
| 320418 | AI674461 | Hs. 199638 | ESTs | 0.47 |
| 302982 | W92391 | Hs. 198222 | ESTs; | 0.48 |
| | | | Weakly similar | |
| | | | to C2H2-type | |
| | | | zinc f | |
| 315609 | AW207535 | Hs. 224012 | ESTs | 0.48 |
| 317056 | AA904908 | Hs. 250643 | ESTs | 0.48 |
| 314361 | AL038765 | Hs. 161304 | ESTs | 0.49 |
| 315169 | AI371390 | Hs. 158667 | ESTs | 0.49 |
| 323743 | AA324992 | Hs. 257168 | ESTs | 0.49 |
| 313903 | AW167439 | Hs. 190651 | ESTs | 0.49 |
| 315061 | AA551196 | Hs. 188952 | ESTs | 0.49 |
| 300969 | AI140799 | Hs. 76230 | ribosomal | 0.5 |
| | | | protein S10 | |
| 331950 | AA454595 | Hs. 99369 | ESTs | 0.5 |
| 315076 | AI623817 | Hs. 168457 | ESTs | 0.5 |
| 300975 | AI283548 | Hs. 149668 | ESTs | 0.5 |

Pkey: Unique Eos probeset identifier number
ExAccn: Exemplar Accession number, Genbank accession number
UnigeneID: Unigene number
Unigene Title: Unigene gene title

TABLE 1-20A

| Pkey | CAT Number | Accesssion |
|---|---|---|
| 108446 | 112224_1 | AA085383 AA126091 AA074174 AA075373 AA079120 AA070831 AA075978 AA075372 AA128503 |
| 108474 | 116896_1 | AA115179 AA079667 AA115897 AA079771 |
| 100635 | 10605_34 | BE259039 W29128 AW410299 X72990 BE246492 NM_005243 X66899 AI909006 AW248151 AL031186 AA012966 BE273549 BE311429 |
| | | BE253102 Y07848 BE538102 BE256863 BE261240 BE312156 BE618412 BE257322 BE620446 AW806629 |
| | | AA376777 AA325384 BE256808 BE251039 BE257878 BE275352 AA357169 AW403562 AA204995 AA093259 W95953 |
| | | BE256279 BE336683 BE252465 BE251266 AA380754 BE294942 AA380941 AA380999 BE297164 BE249995 BE294719 |
| | | BE295372 AI270673 BE305132 BE563752 BE295357 AI525421 BE263980 AA057505 AA020915 BE266318 BE206948 |
| | | AI474020 BE296420 BE297374 BE408545 BE019366 BE407372 BE266180 BE279437 R58233 T19567 BE300738 AW381179 |
| | | AA357571 AW361285 AA436908 AA301019 AA301022 N20202 BE408777 BE548638 BE167415 AA071260 BE088429 BE280092 |
| | | W23117 T19568 R51681 AW402216 W22784 BE185607 AI457224 BE544120 AL134874 S72620 AA375079 D51319 |
| | | AW818280 BE514686 AW853024 BE563744 AA300469 T07592 BE622190 BE272834 W21781 BE315450 BE542367 BE393120 |
| | | AA988441 H55137 BE562296 BE622502 BE395960 AA329733 AA332348 AI768317 AA456866 AI497832 AW878437 AA857042 U18018 |
| | | BE621418 AI818790 AI949507 BE397693 AI885545 AI858854 AI355147 BE169028 S62138 AW732191 AA856891 |
| | | BE266060 X71427 BE268557 AF095890 AW001288 AI799634 AI623498 AA071346 BE547662 BE261446 AI564543 BE559759 |
| | | U35622 BE314249 BE264915 AI638591 AI538385 AW090025 BE384754 AI888689 AW778800 AI925273 AA075797 AW949130 AV660275 |
| | | AW438697 AI587137 AI524121 AA806249 AW628247 AA808241 AI244388 AI761125 AW117672 AA911782 |
| | | AI129250 AA654447 H55291 BE258050 BE206162 W95867 AA857187 AI871378 AI660103 AW103827 AI220929 AW149949 BE465561 |
| | | AI302857 AW168841 D82190 AW249814 AI623432 AI687358 AW951077 R51592 W60458 AI092863 AW474693 |
| | | D12765 AI911646 D82208 D82187 AW074031 AI358527 AW338497 AA970893 AW072573 AA205364 AI858886 AA012830 AW148763 |
| | | AI863056 AA548656 BE250325 AI016994 AI864005 BE046122 AI497746 C75340 R58896 D82141 AW168240 |
| | | C19048 AI741090 D29465 AI222365 AA948288 AI583522 AW572212 AI091290 AA582727 AA579897 AA570629 W60883 |
| | | AW516989 AL038160 AA577334 AI865872 AA994043 AA922583 AA464778 AA209178 AI829479 AI370235 BE246529 |
| | | AA384177 AA456255 AI699730 W60654 AL035744 AA862042 R32756 AI886886 AA993087 AI289479 AA627840 AA464184 |
| | | AI619503 R32755 AW075358 AI432315 AA457024 AA020865 R92132 AA454629 AA746059 AA454643 AA456240 AA826984 |
| | | BE163738 AI806470 AI991074 AI802560 AA587095 AA558714 AA968521 N87780 AI538246 N71794 AV661738 AI368903 |
| | | AA362570 AI989445 AI674962 S75762 BE245204 AA975296 D20123 AW005704 AA693328 AA582270 AI918474 AW205707 |
| | | AI696299 AA220990 AA101538 T29030 H27201 AW262526 AI610530 AA126840 AA126790 X92120 AW367868 BE299644 |
| | | BE299451 AA476561 BE300044 AA134363 BE295222 AA307504 N42337 AA319098 N39502 AW964461 N57241 BE299049 |
| | | N86332 R51156 AA085859 T75212 AA133939 AA147129 AA156161 BE543953 BE538848 AA133676 BE299745 AA135050 AA218535 AW406401 |
| | | AW411287 BE410528 C01410 NM_004083 BE314959 AA836413 AA085862 AW024370 AA471059 |
| | | AW467508 AA001025 AI828231 AA633221 T95517 AA147038 AA476447 AW027012 AW078627 BE513200 AI192297 |
| | | AA886279 AW081806 AA316185 AA010506 AI269929 W93139 AI682935 AA609555 AA378028 AI093877 AA999997 AA730698 |
| | | AI143923 AW575315 AA890550 AA494353 AW576601 AI796336 AA826130 AA609207 AI539618 AI088539 AI089090 AA825505 |
| | | AA632978 AA015892 AW204713 AA156495 AA824613 AA133630 N29826 AA527476 AI633352 T27908 AA134364 AA133940 |
| | | AW043601 H37775 AA772375 AA057871 AA047888 AA054225 H86568 AA001511 H25718 AW189507 AA165589 AA054433 |
| | | H85549 AA165486 AA058972 AA454911 AA464064 AA493802 AA428253 R85508 AW302469 AI611812 BE162582 F11073 |
| | | T95518 N26811 AI783929 H40669 AW611745 AI658803 R51042 R45276 AA528386 AA782875 AW880218 AL138391 AA314536 |
| | | AW949338 AA149466 AA149552 AI346513 AA216776 BE349131 AW007654 AI141803 AA622688 AI185131 AW057635 |
| | | AA101539 AA627986 H27202 AI536847 W93084 AI973148 AI246788 AW572108 AI469414 AA454835 AA612707 AA430746 |
| | | AI084991 AA010400 AA856636 AA463928 AI248310 R07170 AA834033 D12244 AI655670 AA054350 AA639480 AI702067 |
| | | AI475389 |
| 100643 | 3931_1 | NM_005032 M34427 AA332167 AW409711 AL119718 BE297581 BE299855 AA082284 AA226855 AA149568 AW391953 |
| | | M22299 BE163594 AW847881 AW366993 BE142871 AW847885 AW604137 AW847753 AW847886 AW376442 U48350 |
| | | AW607478 AA373011 AA334080 BE294177 AL121355 AA302236 BE540666 BE170588 AA346884 BE541512 AA226818 |
| | | AA082001 AA366490 AW604122 AA205784 AW607791 BE168496 AA058497 T64373 BE165633 AW802804 AW847878 |
| | | AA187408 AA088397 AI751745 AA344103 AA034463 AI906008 AA363580 AA379193 AI332642 AI143569 W52748 W52754 |
| | | AA385532 AA085967 F05943 AA363422 AA133444 AA133477 AA029541 N48387 N83348 AA376066 AA147671 W70187 |
| | | AA316255 BE174987 AA452776 AA089605 AL047776 BE162673 H39532 BE168406 AA357654 AA328728 AW813442 D57844 |
| | | AW839748 AW839663 D57357 AA334536 AW268674 AW950788 AW409888 AI160544 D57821 AW664382 D25884 AI755101 |
| | | AW130365 AI609094 AI984064 AI806523 AA492516 AI755258 BE157210 AA374884 AI983923 AI831088 AA706501 AI754957 AI688651 AI088623 |
| | | AI336114 N38752 T56004 AA845200 AA858377 BE157397 AW069347 AA045366 AW316918 AW130372 |
| | | AI355398 BE157396 AI751746 AI375820 AA129935 W60002 N24781 AI805924 W60009 AA044283 AA121161 AI539277 |
| | | AA301885 AW019944 AA133445 AA101108 AA033559 W70060 AA617751 AI986261 AI023234 D82235 AA085846 AW754181 D82093 D82100 |
| | | AA147653 AA600256 D57884 AI753982 AI568050 AI146490 AW302280 AI433051 AA329188 AW572150 |
| | | AW166345 AI337981 AA778973 N67577 AA227207 AA838281 C06190 AL046997 AI217662 AI752979 AW627538 AI127171 |
| | | AI440461 T64184 AA845190 AA227111 AA877394 R60962 AA505646 AA770545 AI696264 AA953747 AA904094 AA058318 |
| | | D57026 T17158 AA578545 AW085082 BE148939 AW815069 BE152843 BE149068 BE149036 AW815073 AW753691 BE149040 |
| | | AW815065 BE152842 BE149072 AW753692 AW815055 BE152837 BE152849 BE152840 AW815070 BE152829 BE152846 |
| | | BE148972 BE149042 BE149074 AW753668 BE152832 BE152841 BE149082 BE149050 AA347261 BE152852 BE152847 |
| | | R65797 F02189 AA483448 AI954410 AA865375 BE152836 BE152838 BE152839 T17300 BE152844 BE152833 BE152834 |
| | | AA029542 AI567601 AI362353 BE162140 AI381384 BE152851 D57038 D57043 AI418363 AA133478 BE149051 BE149083 |
| | | BE152850 BE149052 BE149084 AA886686 BE149064 BE149032 AA044093 AA129934 AA303976 BE157211 AA187291 |
| | | BE152830 AA046552 BE149047 BE149079 BE149033 BE149065 BE149044 BE149076 BE149053 BE149085 BE149034 BE149066 BE149048 |
| | | BE149080 BE149038 BE149070 BE149045 BE149077 |
| 100670 | 22023_1 | AA332178 BE259177 BE545625 T09105 S62076 M16424 NM_000520 BE244309 F13516 BE251567 BE514981 AL119537 |
| | | AA336739 BE261801 AA278642 N32708 T77034 W24621 W42478 AW630382 AW856214 AA134234 M13520 BE379212 |
| | | AA287459 BE019379 BE297192 BE162970 AW405668 AW403322 BE272280 BE208703 BE304428 BE162807 BE162828 |
| | | BE162887 BE078944 BE163025 BE162878 BE162909 BE162898 BE162791 BE162880 BE073563 BE163086 BE162896 |
| | | BE162770 BE073565 BE162906 BE162913 BE162947 BE162803 BE262199 BE162811 BE080697 BE315095 AW206024 |
| | | AI291054 BE087364 AL046839 AA304422 AA847660 AA669876 BE392765 AI567798 AW026644 AW151258 AA996314 |
| | | AI828660 AI571158 T61941 AW103503 AW172698 AI923115 AI823709 T62167 AW771381 AW151782 AI799284 AW242271 |
| | | AA128031 BE261306 BE312241 AI674880 BE261057 AA630684 AA831305 AI139546 AW082447 AA916854 AA916855 T05970 |
| | | AA599395 AA921680 AI244674 AI041920 AA424998 AI362999 W42543 T51260 AI362486 AI699366 AI827925 AI027381 |
| | | AI027370 AI209049 AA782220 AI334014 AI279051 AI217711 AI674210 AI193370 AI701683 T23782 AI927545 AI784291 |
| | | AA128007 AI370630 AI972736 AA853763 N92379 AI916746 AA639633 AA907603 AI479452 AW950971 T28985 AI685825 |
| | | AA563654 AA745291 AW089417 F10858 AI354227 R38108 AI668647 AA994088 AI740910 AW880973 AI739410 AI480346 N78987 AI473892 |
| | | BE162903 BE254430 BE260426 AA650012 AW006426 |
| 100673 | 21517_2 | AW403342 AW248986 BE561709 AA357312 BE311834 BE389496 BE294887 AW732696 BE047868 AI702383 BE019155 |
| | | AI702367 BE408966 BE280458 BE313759 BE513492 BE535404 BE280258 AC005263 NM_007165 L21990 AW732711 |
| | | AI564920 AW249094 BE265365 AW607186 AW607346 BE005217 H27211 U46230 BE260066 BE207043 BE546782 AW248659 |
| 108559 | 41469_9 | AA085228 AA085161 |
| 108569 | 118606_1 | AA082885 AA114265 AA085398 AA113184 |
| 100700 | 17137_1 | AA932794 BE540417 AW409802 AW410765 BE296651 BE294197 BE164813 AW381886 AW381806 AL048654 AW403058 |
| | | BE207228 AA464654 AW966967 AA326831 BE407277 BE408669 AA476527 AA115576 AA359697 AA476357 AA449939 |
| | | BE263719 AL045304 W21442 R28919 BE395990 AA252273 AI346812 BE538487 AA507160 W93950 N42025 AI088439 |
| | | AL134931 AA031524 AI887287 AW470017 AA476423 AA464553 AW410766 AI569421 AA577476 AI248935 AI912371 |
| | | AW615674 AA824237 AA807746 AA827377 AA890268 AA476309 AI086424 AW409698 AA031553 AW451901 AI520934 |
| | | AW050554 R49825 N30302 AA532541 W94004 N93402 AA115549 AA331202 L25665 NM_005275 AA436745 AI122671 R49779 |
| | | R18508 |
| 100734 | 35197_1 | AI807481 AI500404 AI092260 BE348962 AI143675 AA772399 AA772398 AI368565 AI379172 AI083781 AI871363 AA843793 |
| | | NM_000141 M87770 X52832 M55614 Z71929 W05259 AA548551 AI498743 BE081295 BE162251 F05643 AI127918 H83199 |
| | | W07463 BE551725 R28404 AW206461 AW590506 AI885536 N69800 R93496 R25381 AA443093 AI143063 AI284647 AI703144 |
| | | AA309032 AA309031 AW949426 AW949428 AI638387 AI638356 R77173 R38513 T89263 F01900 H87979 R70205 AA664355 |
| | | AA235910 AA033657 AI436212 N50463 AI433805 AI081876 AI421090 AW020818 AI559529 AI887420 BE154202 AA889062 |
| | | AA723410 BE537575 AA236812 AI218552 AI264866 AI290617 AA424365 AA424505 AW073347 AA032183 AI142488 N55322 |
| | | AI884363 AI336070 N67307 AA608928 T94993 AW514184 AA724695 N66630 AI379638 AI274671 AW628470 AA235346 |
| | | AA687581 AI073906 AI263602 AI869111 AI805693 AI423808 AI076491 AI374640 H82967 AA776567 AA256191 T29856 |
| | | AA953586 AI140801 AI805484 AA984329 R93497 AI017114 AI263355 N81103 AW418776 D57474 AI918460 AA256152 |
| | | AI683268 AI042628 AW196650 C00195 AI918567 T28903 N95383 R13671 T94939 AI275235 AA235751 T84335 |
| 100739 | 2738_3 | M59287 L29222 AI251890 BE244986 AI708332 |
| 117040 | 46956_1 | AW970600 AA503323 H89218 AF086031 H89112 |
| 100748 | 41861_1 | X06096 X05826 |
| 100760 | 1334_7 | AW794626 M27126 M27014 |
| 100779 | 458_127 | BE561958 BE561728 BE397612 BE514391 BE269037 BE514207 BE562381 BE514256 BE514403 BE514250 BE397832 |
| | | BE269598 BE559865 BE396881 BE560031 BE514199 BE560037 BE560454 |
| 100787 | 458_127 | BE561958 BE561728 BE397612 BE514391 BE269037 BE514207 BE562381 BE514256 BE514403 BE514250 BE397832 |
| | | BE269598 BE559865 BE396881 BE560031 BE514199 BE560037 BE560454 |
| 130872 | 21268_1 | U61084 NM_904900 U61083 AI761325 AI826909 H79385 T81886 AI222763 N68038 AI281048 H79274 AA603662 AA721720 |
| | | T71211 C00488 AA994672 AW136970 AW368715 AA380767 AL022318 |
| 108641 | 853_−13 | AA112059 |
| 100818 | 19604_3 | U79251 AA843851 R38201 R66461 R44908 AA683289 H17477 R37364 R52832 AW298336 AA351391 NM_002545 L34774 |
| | | AA296886 AW967001 T28889 R13451 T77331 AL119196 AL118830 H08459 AW892812 AW905838 H17585 R52878 |
| 130930 | 2773_1 | NK_005658 U19261 BE622108 AA313592 AW950162 H25107 R71725 R50630 AI524201 AI476301 AW014547 AW195770 |
| | | AI378122 AI554908 AI927196 AI913959 AW044513 R50534 AI379950 AI311593 BE043305 R82981 AA769375 R77429 |
| | | AW196220 AI269033 AA883433 R71691 F11489 AW771234 AA402642 AA399408 AW771244 AI400707 R55446 |
| 124394 | 5590_5 | AI950447 AW027427 AI640151 AI139433 AI400708 AW779975 AI739122 AI384000 AW079410 AI473425 AA150109 AI766579 |
| | | AI351784 AI209046 AI474732 N29724 AI382653 |
| 100882 | 458_127 | BE561958 BE561728 BE397612 BE514391 BE269037 BE514207 BE562381 BE514256 BE514403 BE514250 BE397832 |
| | | BE269598 BE559865 BE396881 BE560031 BE514199 BE560037 BE560454 |
| 100885 | 12707_3 | X07881 NM_006249 X07637 AA376715 AA376677 X07715 X07704 S80916 |
| 100896 | 205_6 | M91803 X65362 |
| 100898 | 8542_1 | BE387614 R51501 AA199714 AW674779 F08178 BE269071 AA376313 H08264 AA380420 H18785 AL042151 BE277758 |
| | | BE267438 NM_005850 L35013 BE540833 BE390902 BE391494 BE277459 BE385592 BE390612 BE384263 BE387779 |
| | | BE388647 BE537373 BE547158 AW409585 AW374033 AW602185 AA355725 AW577548 AW935015 AW935160 W40232 |
| | | AW938647 AW374332 AA434040 BE293488 AL138361 BE560260 AI745075 AA317980 AW949382 AI834311 AI653582 |
| | | AI831042 AI361878 AA618606 AA729052 AI424969 AA199715 AW769374 AI828422 AW044307 AI862816 AI203583 AW084461 |
| | | AW514655 AA831883 AA290672 AA831286 AA578510 AW089965 AW150746 AA292743 H22232 AI469275 AW439312 |
| | | AA292744 AW471443 AI473989 AA593336 AA464070 AI678937 AW069451 AA970763 AA610480 AA593328 AA464009 |
| | | AA768985 AI298928 AA436600 AA464718 AA699361 D61482 D55935 AI369591 AA470695 AI809135 AA640627 AI568446 |
| | | R51502 W45467 AI655316 AA463934 AW168609 AW518663 BE045525 Z41251 AI868091 AA908160 AI026697 AI886259 |
| | | AI612932 AA215437 AI956014 BE541087 BE255652 BE265878 BE394102 W27502 |
| 108706 | 13255_−3 | AA121820 |
| 108710 | 133560_1 | AA121959 AA121960 |
| 108712 | 14671_1 | AA335738 AI817426 AA099503 AA643106 AA650582 AW188499 AA155785 AW024305 AA992590 AW793619 AA121250 |
| | | H93101 AA994582 AW007456 AW799974 AA535738 AI460362 AI571942 AW517220 BE044613 AI830018 H50382 AW391334 |
| | | AA741035 AI697291 W37616 AA866185 AW264926 H52854 AW796893 AI660673 AA464961 AI808376 H13850 H81969 |
| | | W92615 H41636 H73900 H66731 AA281556 AA026801 AA479538 AA588901 AA446262 R86974 H84761 R62958 H70493 |
| | | AI991716 AI833075 AW800109 R54692 W94542 R78651 AI694890 AW845181 W31325 AA224371 AA359813 AW178868 |
| | | H53738 F22305 AI874075 AI567440 AA853303 H25886 AA550734 AI874064 AW148463 AI586928 T28979 AW950966 AI828723 |
| | | AI918890 F16942 AA223763 AA622370 AA630985 AA402644 N70971 AI147210 AW410026 AA485517 AI129376 AW337522 |
| | | AW136362 AI123362 AI281243 AW339600 AI478990 AI339256 AW768289 AI660065 AI309560 AI141111 AI280844 AI338227 |
| | | AI310719 AA733112 AA903203 AI073351 AI348225 AA974151 AI141386 AI311925 AI042124 AI193176 AI186999 AI735528 |
| | | AI335702 BE550806 W47157 W70086 W44788 AI218741 AI074049 AI168088 AW050904 AI083978 AI131084 AW328532 |
| | | AI969255 AA069140 AA328735 F20431 AI625929 AA621514 AA384576 H98823 AI371848 AI360520 AW079514 AW845177 |
| | | AA948626 N52236 AA464158 H43126 AA465035 AA464221 AA365164 AI539264 AW250430 N27769 AA157036 AI124029 |
| | | AI635488 AA374090 AA515126 AA614763 AI872210 AI160916 AI002016 AA813669 AA190550 AI262671 AI460058 AI690719 |
| | | AA805473 AA523207 AA477649 H13898 AI056086 H42356 R32267 R99431 AA481898 AI130779 AA769777 AA910293 |
| | | AI284079 AA936692 AA903953 AA947753 AI568924 AA902966 AA807082 AA489337 N58247 AA872223 AA485362 AA024906 |
| | | AI092224 AA443621 AA603059 N58248 AA927262 AA329524 AA026369 N73450 AI244031 AI192676 N25190 W32248 W32324 |
| | | AI284307 AA074470 N33116 AA947399 H52855 T52343 AA643260 AA573752 AI031889 AA877690 AA531409 X13710 |
| | | AI500538 R32315 BE168562 AA401993 AA883251 W37615 AA305694 W78964 AA024905 H53678 W47278 AA224423 |
| | | AA371881 AA335679 AA336008 T52416 H69646 AA300266 AA325728 N73204 N77626 R54691 W24032 AA868340 BE407766 |
| | | H81968 W46665 AA883934 AA187290 AW571425 AI280505 AI079662 AI470373 AA991617 AA173165 AI244032 AA922440 |
| | | AA856918 W80458 AA121251 AI160062 H50349 T67744 W46574 AA969287 W01485 BE615302 BE388184 AA314246 H85693 |
| | | W31948 AV650437 AW160467 W44891 AA723773 AW328531 AA313224 AA299527 R86894 AA312505 H21582 AA157493 |
| | | H78480 N36060 N77627 BE218674 W02853 AW954844 AA429974 BE382847 AI541225 H93229 BE281553 AA147790 |
| | | AA327374 AW839840 AA969229 R99337 BE278812 AI190450 AA479549 AA156012 R78705 AA224081 N44944 D56066 |
| | | AA969012 AW178878 AW105500 N52391 AA622301 AA858204 AI290372 AI205676 H51289 AA548013 BE544240 AA280160 |
| | | BE170901 AI192429 R77056 AA310925 AI219128 AA121993 AA070432 R63012 |
| 108785 | 4962_−6 | AA128946 |
| 102156 | 33676_1 | H08948 AA353935 R14985 U17977 H15387 F05567 R88476 AW968314 R89646 R87941 AA258917 AW895155 AW947450 |
| | | AA223175 AA223559 W92508 W02327 AA322882 AW960879 H39040 H39039 R90891 AA580343 BE155037 BE154984 N40110 |
| | | AL137683 BE265131 R17329 AL096733 M79202 AA625234 AA427472 AW361296 AA658045 AA322177 R54596 AA171880 |
| | | AL042331 U18920 AA298036 AW051888 AI378803 AW601640 AI885103 AA297019 AI192706 AW055183 BE312004 H42382 |
| | | AA534726 AA205943 AA204817 AA298966 AW015511 AA223176 H08864 AW028026 R45676 AI023345 AW263046 AI161297 |
| | | AI168747 AW024885 AI832106 R88436 R86849 W92509 AA857908 N26989 AI589169 AI570370 AA223525 AA171552 |
| | | AA405862 AW001095 AI864651 H39060 AA776342 AI682761 AA768233 R86855 AI367878 R42675 AA832040 R89042 |
| | | AI984848 AI524629 AI289021 AW014020 AI241757 AI421241 AA430654 AI360728 AW299692 AA258155 AA782959 AI684411 |
| | | AA531501 H41286 AI125231 H15388 M62002 R88819 AI203108 Z38537 AI432593 AA887767 AW023638 H39016 AI424700 |
| 102186 | 33745_1 | U20285 AW674959 W69617 NM_004127 AU076538 BE245805 AW250694 AW248164 BE294678 AA298083 BE266030 |
| | | BE262348 AL038115 BE253256 AA330082 AW401492 BE070159 BE619216 AW379955 AW379968 BE266381 AL046753 |
| | | AW379969 Z45804 H12155 BE543514 Z44978 AA026010 AA776140 AA337940 AA315980 AA308668 BE156554 R33383 |
| | | BE271569 BE378456 AA972517 AW748739 R73336 AA302223 AA279618 BE561802 AI693878 BE560858 BE513865 BE267975 |
| | | R36064 AI056387 AI961737 AW167515 AA280965 AI479985 AI859762 AA309009 AA573934 AI669053 BE046497 H14545 |
| | | BE222639 AI910094 AA484398 AA521025 AA807224 AW247126 AW732050 AA377850 AW951499 R73316 AI458542 AI564292 |
| | | AA974663 AI951858 AA595162 AI685726 AA604679 AI813703 AI091930 AI567797 AI521316 AI377236 AA583446 W79901 |
| | | AI743281 AA913078 AI139695 AA994505 AI202810 AA662524 AI810703 AA988395 AI272746 AA569807 AI299166 AI799551 |
| | | AI492268 AI131335 AA969251 AI240890 AA580208 AI928074 AA716051 AA657992 AA988396 AA994504 AI952561 AW188344 |
| | | AW246831 AI471309 AA757891 AA931856 AI272794 AA766100 F24931 F24930 AA749404 AA708597 AA732155 AI796670 |
| | | AI825867 BE245682 AI638230 AA434157 AW249881 AA481945 AI090239 AA923622 R72864 F25087 AW674940 AI808546 |
| | | AI470209 AI285895 AW189219 AW474426 AI818261 AW300960 AW513345 AW073126 AI868353 AA862926 AW592528 |
| | | AA502647 AW591711 AI354982 R49145 AI360724 AI458656 AW304052 Z41443 AI864783 AW651615 AI270639 AW316592 |
| | | R43741 AA482068 R72852 AW190442 AW651631 AW470902 AA911918 AW779251 R72820 AA279995 AI394715 AW105688 |
| | | AI097434 AA670473 F08806 AA035597 AW008688 AI689117 AI911859 BE387696 AW166880 AA847278 W78016 AI886548 |
| | | N35512 T86152 |
| 125077 | 1814028_1 | T88822 T80794 |
| 124440 | 22853_1 | AA532519 AA770627 BE326342 AW073299 AW205539 Z83851 AA725380 AI457288 AA976882 BE220181 AA988940 AI914073 |
| | | N46435 |
| 100941 | 3303_2 | U15422 NM_005425 X63758 X63759 Z46940 L03378 AI220577 AA634325 AI381479 AA994994 AA994988 AA416961 |
| 117286 | 558874_1 | AI188710 AI032142 AW078833 N30308 AW675632 AI219028 AI341201 N22181 H95390 |
| 117291 | 1047256_1 | N81189 AW172914 N22182 N22289 |
| 132316 | 10556_2 | U28831 AA088359 N56023 AA069486 AL038872 AI683185 AA632516 AI281643 AA447146 AI699884 AI281642 AA832250 |
| | | AI909017 AA781851 BE090060 AA058888 AI440090 AA040563 AA069449 AI702237 AA045495 AA088258 AA229820 AI925213 |
| | | AA928183 N90591 AW794070 AI884470 AA219147 AI632560 AA553416 T10311 |
| 102295 | 26281_1 | AB007881 U32581 AW131202 AW995994 W31964 N24261 AI033045 H98694 AW364848 AI222031 AA907216 AI215730 |
| | | AA776981 AW473826 W31373 |
| 132380 | 166896_2 | AW373665 AW373648 AW373647 AW393531 AA284401 AA253299 BE168092 AA196249 AA810695 AA278353 AI806177 |
| | | BE552269 AW001862 AA749299 AA253300 W37080 AA805586 H72533 AI698623 AA292703 AA748852 H75291 H68018 |
| | | AA883728 N48304 N49030 BE168089 |
| 102337 | 553_1 | AI814863 AA806761 AA765241 AA019317 AA092255 AA035405 T85079 AA890151 AI373959 T85080 BE153728 AA740848 |
| | | BE080682 AL048137 AW182316 AI699488 AW274481 AW407538 AA306562 AW950024 AW949943 AL045703 AW843196 |
| | | W25132 BE612794 AA304266 AW958054 H25673 AV646563 AV648573 BE172990 AW593488 AA385181 AA164998 AI246476 |
| | | AA345406 AI277554 AA134749 AA856624 BE613247 AA299003 AL048138 AA028121 T92510 AI923835 AW020440 AI401594 |
| | | AI889401 N93290 AA044247 AA028100 AI582845 AA811151 AI741811 AI925878 AA448277 AA172221 AI214783 BE220793 |
| | | AA022746 AI082882 AA022849 AI928385 AA573472 AI420686 AW072902 AI799493 AI873506 AI468977 AI192079 AI468976 |
| | | AA044272 AW015701 AW316979 AA933042 AA609017 AI318393 AI424571 AI934945 AA172023 AW050917 AA846180 |
| | | AA134748 AI003947 AI766789 AW006697 AA653517 AW575680 AI474214 AA401478 U36922 AA927064 AA868000 D62654 |
| | | T91745 AW500202 AA194764 AA746346 AA130464 AW117498 AA054526 N26432 H02534 H04964 AW303367 BE300931 |
| | | AI218049 AI208073 AW182749 AA983630 AI147585 AA194765 AA054534 AA922720 AI436585 AI346535 AA134269 AA280923 |
| | | AA897422 AA019559 AW274010 AA035406 AA917879 H99327 W32908 AI216046 AW496823 AA019414 H82288 W35284 |
| | | AI936621 AI767113 AA866177 AW367874 H82398 AF032885 AW300151 AW467069 AA809346 AI188507 AI494178 AA872752 |
| | | AI631631 U02310 NM_002015 AA815006 AI382453 AW197658 AI761654 AI804396 AI382221 AI813640 AI439635 AI523901 |
| | | AW517242 AI221705 AW298104 AW204560 AW573095 AW028783 AW014650 AI766744 AI808294 AI698758 AI041809 |
| | | AI766667 AI479103 AA872797 AA769305 AA765080 AA334166 AI472322 |
| 131862 | 3673_14 | AI541246 AW055046 BE312897 NM_006623 AF006043 AL038012 BE256739 BE272577 AA143633 AA143643 BE464595 |
| | | AI312112 H01791 R26319 BE170902 AW131612 F29120 AA455866 AI540181 AI885777 F36716 AI149501 AI246028 AI373003 |
| | | AL046700 AA455869 W69120 AA146617 AI188256 AI937281 AI806751 AI087240 AI123219 AI589686 AI332501 W69087 |
| | | AI494321 AA725793 AI222350 AA625736 AA644568 AI472282 AI370905 AA934652 AA913035 AI128576 AW341709 AI269125 |
| | | AA975139 AI620890 AI969828 AI356660 W95879 F32252 AI218442 AI928601 AI474470 AI245409 AI601111 AA928594 |
| | | AI809316 F19498 AI373442 AI218893 AA573914 AA906869 AI833041 AA993899 AW250763 AA887142 AA884655 R26108 |
| | | AI919414 H01042 AA534622 AA723368 AW005753 AI871394 AA661885 BE171317 BE271194 AW249310 T83902 BE246962 |
| | | BE279817 BE562094 BE513363 AL045369 BE561008 BE267526 BE544065 BE559579 BE561747 T35796 BE250596 BE268704 |
| | | T31298 BE536521 BE561183 BE269560 BE269944 BE514005 BE561068 BE268145 BE267559 BE559744 BE560930 BE278335 |
| | | BE513832 BE561017 BE396371 BE562260 BE281009 BE281327 AW410446 BE259628 BE547443 BE311971 T83809 |
| | | AW249441 BE270204 BE295488 R90868 BE269359 BE397222 BE396694 AW248394 BE397708 BE268174 AW247676 |
| | | AW248404 AA093721 BE397622 AA093706 T09027 BE408338 BE261454 BE260628 BE295110 AF171237 BE295135 |
| | | BE407534 BE536635 BE264727 BE296052 BE270265 BE546712 BE298269 BE547732 BE548734 BE253463 BE312238 |
| | | BE261042 BE545186 BE313055 BE542602 BE538530 BE535215 BE208107 BE513616 BE295982 BE561273 BE560057 |
| | | BE562005 BE280371 AW247443 BE259311 AA126209 AA171445 AW674121 AA134790 BE018733 AA233357 AA121604 |
| | | AA114201 BE265626 BE260086 BE313195 BE313434 BE261587 AA223432 AL039212 BE260762 AL046154 BE549057 |
| | | AA316523 BE259727 BE260759 BE208754 BE019077 AA206899 T33021 BE255340 AA182643 BE258057 BE258040 BE254759 |
| | | BE544454 AF171236 AA224410 T08700 AL037155 BE279638 W78185 BE545040 BE261376 BE261692 BE259694 AA313254 |
| | | H09056 AA357719 BE619163 BE018472 BE277966 BE278390 AA353616 AA378769 AA356685 BE564039 BE300236 |
| | | BE250358 BE543245 AF171235 BE384461 BE408141 AA137039 AI355285 BE535442 AA356783 AW675020 AI810351 W17380 |
| | | BE074695 AI978750 AA316052 BE278523 BE619725 BE270953 AW026987 R89415 BE271359 BE515234 AW008497 |
| | | AW386938 AI038839 AI862998 AI719458 AA113414 AI859984 AI870773 AA129452 BE047253 AW673881 AA827034 BE302302 |
| | | AI572915 AW264575 BE294567 AA805899 AI625116 AA310115 AI460065 AA169760 AA955009 BE302084 AA629846 |
| | | AA640597 W63787 AI805738 W94207 AA114165 AI240462 BE349066 AA668794 AA134085 AW050730 AA134084 BE540728 |
| | | AA910015 AW248892 AW247299 AA564678 AI139117 T16008 AI754100 AI091987 AA856537 BE205770 AI199445 AA603509 |
| | | AW771741 AI126994 AI000820 AI023844 AW250047 AI673263 T35536 AA126049 AI668802 T35470 AA224348 BE247113 |
| | | AI367665 AA223233 AA206815 AI284773 AA093396 AA113268 T03547 AA234268 AA101404 AA991274 AI275931 AA918855 |
| | | AA236365 AA226966 AI685723 AI284774 AW248023 AI754314 H09057 AA844271 AI272992 AA315077 AA205718 D57244 |
| | | AA742550 AA219567 AI927435 AI042620 AW247957 T35545 T15567 R90845 AA719914 AI085499 AI970845 |
| 117435 | 23179_1 | AF151074 AF070529 AI969222 AW874125 AI198308 AW470316 AI079388 AW134783 AW206208 AI093223 AI924789 N26130 |
| | | AI934430 AI857979 AA928091 NM_016496 AI074817 AA663800 AW300768 AW264438 AI884506 AI025192 N27628 AI199456 |
| | | BE044999 AI277491 BE044964 AA095316 AI076515 AA836677 AI942491 T80147 AI417945 AI668895 |
| 102459 | 3556_1 | U48936 L36592 X87160 NM_001039 AL036606 AL036420 U35630 AW298574 |
| 125324 | 1692163_1 | R07785 T85948 T86972 |
| 101809 | 32963_1 | M86849 AA315280 NM_004004 AA315269 BE142653 AA461400 AW802042 BE152893 AW383155 AA490688 AW117930 |
| | | AW384563 AW384544 AW384566 AW378307 AW378323 AW839085 AA257102 AW378317 AW276060 AW271245 AW378298 |
| | | AW384497 AI598114 AW264544 AI018136 AW021810 AA961504 AW086214 AW771489 AW192483 AI290266 AW192488 |
| | | AW384490 AW007451 AW890895 AA554460 AA613715 AW020066 AI783695 AI589498 AI917637 AW264471 AW384491 |
| | | AI816732 AW368530 AW368521 AW368463 AA461087 AI341438 AI970613 AI040737 AI418400 AA947181 AA962716 AI280695 |
| | | AW769275 AW023591 AI160977 AA055400 N71882 AA490466 AW243772 AW316636 AI076554 AW511702 N69323 H88912 |
| | | AA257017 AI952506 H88913 AI912481 AA600714 BE465701 N64149 C00523 N64240 AA677120 |
| 125375 | 16354_−4 | H72971 |
| 126008 | 190485_1 | AA827860 AA253460 AW971543 |
| 126027 | 327799_2 | M61982 AA491766 |
| 103163 | 43223_1 | AU077018 X67683 |
| 102515 | 10404_1 | U89337 AF019413 Y17868 AL044866 M25813 AL042665 N77388 R00971 BE392997 W52502 AW797536 X71937 BE394176 |
| | | T29553 AA114976 AA114835 AW316671 AI799888 BE044614 W51953 AI498690 AW580466 AI972232 T58430 AW137401 |
| | | T58462 AI870560 AI696138 F33285 U52696 Y13782 AW292936 AW375206 AW751873 AW352417 AW375162 AW375201 |
| | | AW375188 AW375198 AW375178 AI024335 AW375169 X71927 U52701 |
| 126055 | 14930_1 | H65527 N28990 AA359399 N24306 H65329 AA056928 AW504127 AA486661 AA040017 AA634817 AA595340 T93478 |
| | | AW015508 C02093 BE513950 AW410741 AW193200 AA813934 AW439574 AI077876 AI954356 AW629350 AI612865 |
| | | AW675663 AA226255 AI816714 AA010653 AA973778 AW276616 AI277607 AI218492 AW275190 AA911289 AI302550 N53061 |
| | | AI244408 AA709434 AA876151 AI206461 AI354477 AA954030 AI241127 AI187313 AW440434 AW074284 AI265804 AA872499 |
| | | AI202853 AA906037 AI241581 AA134769 AW074122 AI004585 BE300852 AA873196 AI813620 AW504945 BE619639 |
| | | AA037624 AA641954 AA037625 AW814771 AA134768 N73248 R02524 AA226533 AA216115 R29049 |
| 126065 | 34268_1 | AA221032 AK001651 AW732625 BE140495 AI366484 BE297579 AL039675 AL042801 AI678076 AL042802 BE208853 |
| | | AI961484 AW190561 AW027201 AA864499 AI344032 AI432437 AI934618 AA572961 AI499074 AW769938 AA917094 AI955647 |
| | | AI342638 AW974120 AI761488 AI289643 AI564533 AW272378 AI796156 AI867205 AA884306 AI766564 AA456581 AW769937 |
| | | AA713594 AI383037 F24965 AL137599 BE259667 BE313085 BE383358 AA322347 H67555 AA298993 AA304712 AA377693 |
| | | BE327057 AL041340 BE273248 |
| 132640 | 179_1 | AW162087 AA224538 AA471218 AA088655 AA375275 BE440052 AF090891 AA324435 AF063549 AI110675 AA322223 |
| | | AW953306 AA233590 AW949864 AW949859 AA383721 AA081878 U33821 NM_006024 AA350900 AA081588 AI148087 |
| | | AF268075 AA088185 AI142478 AA081824 AI887930 AA070570 BE185248 AI459825 BE257794 AA420459 AA420859 |
| | | AA777997 AA081219 AW815721 AW854758 AA157932 BE018208 AW378974 AL041212 AI247564 AW581897 AI002897 |
| | | BE543242 AI811690 AW852076 AW852270 AA360969 AA094943 AA090680 AW601554 AA099673 AA662226 AA356814 |
| | | AA330174 AA187544 C02751 AA315460 BE168358 AW080447 AI813764 AI624222 AW156901 AI954032 AW473780 AI861975 |
| | | AA173643 AW511541 AI951492 BE301686 AA669760 BE182212 AA081009 T69431 AI186207 AA604124 AA707346 AA173953 |
| | | AI016700 AI125916 AA358962 AI673719 T90593 T90497 T10776 AW513002 AW304292 AA724885 AW474759 AI811621 |
| | | AW068925 AA666305 AI580161 AI128023 AW471151 AA534849 AA666358 AI078833 AI139223 AI244874 AI381658 AW263441 |
| | | AI432440 AW802882 N66401 AA224251 AI167469 AI141060 AA099214 AI537130 AL120428 AA948655 D53110 AA076099 |
| | | AA938617 AA826543 AI357914 AA565098 AA807994 AI288812 AA632832 AA157933 AA639802 AA634268 AA282337 |
| | | AA551431 AA557374 AA256923 AA872943 AA009665 H89626 AA810386 T92925 T36145 AA632190 AA130436 AI686635 |
| | | AA130437 AW392904 AW392839 AW392848 AW392836 AA729737 AA070450 AW392890 W04825 AA771848 AA084634 |
| | | AA481985 AI263840 AI801006 AA235380 AI954229 AI559330 AI208724 AA887638 T25894 AA041269 W44443 AI581770 |
| | | W46171 AA878485 W46535 AA197336 AA894945 AA394224 AI766834 AI582590 AI033007 AA481889 AW190598 AW392855 |
| | | R27279 AA398137 AI248407 AI241386 AI991753 AI826585 AA865699 AI096806 AI833030 AA041279 AW888745 AI703279 |
| | | N70572 AI912553 BE549931 AI240422 AW376187 AW591692 AA975905 AW614967 AA009666 W44332 AA664659 T06561 |
| | | BE468150 AI650695 AA587920 AI473310 AI032991 AA256499 AW104241 BE163782 AI984973 BE163613 AI263906 AA628191 |
| | | AA282072 BE163769 BE163775 AI492939 AI473315 D56907 AA587930 H89480 AI362373 AA598483 D56595 AI167590 C16223 |
| | | AI935415 D62555 D62884 D63130 AI760286 AI650286 AW173598 AI499145 AI122566 AW903408 AI810569 AA854936 |
| | | BE049510 D62065 D61900 D62101 R27475 AI469835 AI669086 N80399 N48922 N48746 AA481381 R22858 H13912 AC004549 |
| | | AW602500 AW768788 |
| 102571 | 31561_1 | U60115 NM_001449 U29538 F01103 U60118 AW612050 F33160 F37125 AI138362 AA853999 AI700031 AA868700 AA411791 |
| | | AA782026 AA861693 AA430022 F29006 AI026854 AA236496 AA100266 R60856 AW606023 AW380619 BE150801 AW391508 |
| | | AI682304 AA947687 AI284419 AA782989 F00277 AI202147 F01221 BE161576 AW385226 AW361913 R30713 AW607124 |
| | | AA505577 W96539 T74511 AA211624 Z41920 AA263163 AA216297 BE164746 H56334 W03218 H61947 AA101716 AA134667 |
| | | Z19275 N55888 AA702365 T30562 F00587 AW967294 R58121 W47367 AW608017 N45005 N42952 N69255 AA179110 |
| | | AA196349 AW376390 Z19411 H56335 AA197096 AA651681 AA873207 H61734 BE140426 AA047105 R64437 W45585 |
| | | AA149058 AW984577 AA192500 AW984588 AW984523 AA037874 W93436 AA195586 Z19330 AA523870 T92443 AA149059 |
| | | BE166015 AI033716 AA725097 AA456394 AA180784 W02792 AI921585 AA211488 AA192626 AW364704 AI819174 AA193240 |
| | | AA130173 AI337854 AA181265 W45548 AA779753 AA211417 AA197031 AA037875 AW469501 AA037241 AW024276 F32512 |
| | | AI084805 BE169896 AA192242 R60857 AI089511 F31828 N20892 AI335027 F32813 F22400 F27858 F31766 F33413 AA192610 |
| | | W96433 AI394501 AI150206 AI827777 AW130567 AI932997 T17424 AI288559 R52402 AI806130 AA126040 AA669576 F25175 |
| | | AI148159 AI041869 AA860296 F25152 AI948570 AA479422 F21920 AA778131 AI276508 AI948577 AA114870 F32631 |
| | | AW028047 F35241 AI373951 AA114869 AW138423 AI889537 F24663 F27705 F37165 AI392774 F29432 AI090589 AA450321 |
| | | F24561 W93346 F29070 F25579 F31362 F33440 F22652 F31494 BE169905 AI818513 AI680754 F36378 N38871 F22082 |
| | | F31950 AA665830 AI765076 AA868225 F22733 F35957 F33418 F34871 AA085944 AA035499 F28471 AI653552 BE047474 |
| | | AI362282 AI131185 AI763052 AI028105 AA211569 AW241290 AI086746 F33041 AI767780 AI080600 AA101717 AA086422 |
| | | F32855 AA047242 F33893 F29113 F21510 F21509 AA039540 F26344 F29047 AI937919 W19338 N40995 F31929 AI084510 |
| | | AA026507 AA086200 AA207130 AI399877 F21370 AA733197 AA086205 AW340573 F33682 H05220 F25782 F25048 AI127208 |
| | | F29940 W47368 Z19261 N29900 F25141 F32890 R63826 F35206 F30119 AA706742 F35143 F25142 F33599 AI659403 F25821 |
| | | AA216171 AA991774 F22761 F34089 AW082748 AI093709 F29359 W46627 F26632 F30291 F36082 F20945 F30582 F22531 |
| | | F24027 F33960 AW028172 F24674 F36597 F21959 AA729612 F34366 AA732967 AI582660 F29363 F22167 AI086792 |
| | | AI804183 F33102 AW075510 AI367611 AI784054 AI453418 F34901 AI161157 F26622 AA651987 F35237 AA013303 F32632 |
| | | AA664876 F21736 AA928616 F21384 F25357 F23467 F31185 AI537850 AI868952 F31707 AW089232 AI363826 AI702766 |
| | | AA136298 AI767887 AW089312 AA578225 AA130181 F19251 AA581649 AA449418 F21171 AA455925 AA450322 AA196199 |
| | | AW007853 F26059 AW085130 AI202616 AI674315 F20318 AA747182 Z38215 AW075230 AI159784 AA134668 F00420 F33094 |
| | | AA455054 AA729265 F04647 AA746994 AI658702 AA478718 AA578161 AI080025 AI352604 AA989440 H30904 Z28506 |
| | | F22649 F34880 D45580 C20561 T32638 AA020946 AI439878 AA746991 AA194883 AI219226 Z45773 AA089657 AA180825 |
| | | W25548 R58180 W46675 N38870 AA137104 AA026506 AW984583 AW984539 AA020945 AA035101 AA039539 F30993 |
| | | H14793 AA211688 T91484 F34313 AW815933 F29167 F31099 F31949 |
| 101923 | 30543_1 | X99133 X83006 W38398 AA401137 AA298242 AA366738 AA308126 AW583781 AA298668 AW845024 BE140204 AW845005 |
| | | U47734 AA837575 NM_005564 AA329732 AA421943 BE171567 S75256 AI750047 AI762213 AA100735 AW612993 AI474120 |
| | | AW062884 AI940001 AW062852 AW062899 BE182639 AW778875 AA528093 AW517424 AI939989 AA076188 BE182636 |
| | | AA169569 AA167439 AI283987 AA167783 AA076140 AI749649 AA166792 AI708618 AA400973 AA514773 AA514789 |
| | | AA164458 AA167440 AA074845 AA421944 AA514874 AA079557 AA102361 AA587027 AA642930 AA878029 AA164459 |
| | | AW176400 AW475086 AA857522 AA148193 AA838234 AA593897 AI284506 AW193324 AA148194 AW583341 AI669077 |
| | | AW264913 AA074902 AI680515 AA169874 AA169614 AA079651 AW591737 AW190644 AA076565 AA662747 AA075896 |
| | | AA535642 N27757 AI306666 AA074727 N79823 AA524360 AI826800 AA173827 BE140374 BE004062 AW265060 BE184103 |
| | | AI199258 AA857853 AA299459 AA837890 AI626104 AA503624 BE183618 BE183717 AA573267 AI833071 AW270590 |
| | | AA506601 BE004010 AA837854 AI675895 AI810491 AI184883 AW664712 AA076046 AA515574 AW352267 AI797418 |
| | | AA172395 AI749194 AI559933 AA502597 AA321220 AI866124 AI695633 AA494293 AW085635 AA165649 AA165663 |
| 126117 | 1610460_1 | W01520 H78617 |
| 126122 | 82445_1 | H80181 AA005287 |
| 126136 | 285360_1 | H85525 H83353 AA418447 |
| 126168 | 20960_4 | L44530 AA322034 T79009 |
| 126196 | 21198_2 | AA084092 AA084394 |
| 102675 | 5145_4 | U72512 T98357 R31335 F18090 |
| 118387 | 65081_−5 | N64579 |
| 126256 | 10840_1 | AK001774 R02029 Z21327 R02030 Z21124 |
| 126291 | 86990_1 | AA017609 N42090 AA984485 BE177239 AI912269 |
| 104008 | 27385_1 | AW134482 N93060 AI694673 AW241458 AI161320 AI582177 AI623166 AI492337 AA724337 AA749177 AA741055 AA283954 |
| | | W38395 W24693 AB033047 AA159179 AA588310 AA654615 AA630891 AA639240 AL121157 N79820 AA653941 AA279925 |
| | | AA132883 AA253947 BE502673 AI191149 AF208847 NM_016620 AA132773 AA532724 AA933782 AW997589 AA749442 |
| | | AA356531 AK001587 AW500170 AA369990 AW582815 AW582838 AA451773 AA233995 AA427982 AA334630 AA282933 |
| | | BE349308 AA300682 AI384093 AW364014 R31977 R69849 AW364012 AW364015 AW364019 N76465 R80458 AI267509 |
| | | AI760785 AW628405 AI224905 N59362 AI351352 AA513728 AA729606 R80659 AA573139 AW614666 AA310325 AW471080 |
| | | AI091339 AA830587 AI051323 N67319 AA621436 AA449856 AA648966 AA954973 N51766 AI952427 AA829053 AI468601 |
| | | AA649186 AW592210 AA449968 AA465374 AI474865 AI185456 R31932 AA621108 AA975015 AL121156 AW962752 R69850 |
| 103417 | 15279_1 | L34657 M28526 NM_000442 M37780 AW407571 AA488822 AF281299 AF281296 AF281297 AF281295 AF281301 AF281298 |
| | | AF281287 AF281294 AF281293 AF281289 AF281288 AF281290 AF281291 AF281292 AF281300 BE140137 AA082477 |
| | | AA182953 AI902545 BE185251 D58483 T73117 AW902845 AA303912 AW403488 AA039356 AW401604 S66450 AW371090 |
| | | AA477959 T97049 AA486710 AW607916 R76264 AA303959 AA385484 AA181062 AI218959 AI384084 AW387030 BE044508 |
| | | AI942423 AI910438 W04423 AI148494 D58775 C18167 C18011 R68293 AW340705 AI587613 AW772278 AI942486 AA304612 |
| | | AW276352 N41586 AI248619 AA151818 AW593959 AW137371 AI470913 AI916891 AA181063 AW261863 AI271376 AW007613 |
| | | AA359765 AW874428 AI033514 AA181110 AA181092 H57149 AW514341 AI971318 R95876 AA713790 W93076 AI936184 |
| | | AW665574 R82381 R82269 R53795 R26391 AA989385 AI076568 R68262 AA716613 AA933639 AA678079 AI686366 |
| | | AW294984 N30812 AA486245 AA693877 T95097 T72992 AW193473 AA001726 AI808737 AI247488 N53545 R08174 AA305676 |
| | | N76004 AA761616 R78126 AA564715 AI031710 AI913454 AI475239 AI081733 AW264881 AW518741 AI783794 AA449390 |
| | | C15249 AI922914 AI498549 AI808250 AA486711 AA002005 AA035777 AA151817 AI834300 AI168387 T96940 AI675272 |
| | | AA056355 AA299130 AI127297 T95197 AI818125 X96849 AA486244 W93075 H57223 AA302220 R22071 R33251 AA301548 |
| | | R95875 AA372192 AW838188 H01353 R78125 R22459 |
| 103425 | 30014_1 | AB016092 AB016089 AW161159 AW160794 AW163150 AA776677 AW168207 AW161625 AW157477 AW157176 AI739521 |
| | | AI906428 AW073890 AW467016 AI745171 AA738238 AW090343 BE245923 AW002703 AW293743 AA249074 AW328076 |
| | | AW328075 AF161338 AI991472 AW515714 AI655953 AW393827 AI653282 AI652717 AW393832 BE550526 AI654896 AI203310 |
| | | AW590658 BE550597 AW390889 AW390900 N93494 AI654677 AW573041 AB016087 AI874387 Z28839 AW806640 BE168075 |
| | | Z25196 AA743746 AI906104 AW402640 AA833814 W03404 AW881095 AI863478 BE019680 BE163120 AW939109 AW939159 |
| | | AB016090 AW939112 AW939162 AW939157 AW939107 AA229457 AB002322 BE081002 AW895217 AW367778 AW502353 |
| | | AW502650 AW372535 AW372524 AW372551 AW372527 AW372544 AW893113 BE147617 AA347209 AA772783 AW892035 |
| | | H38889 T19958 T19957 BE171421 AI902871 M78867 BE167584 AI902950 AW862975 AB016091 BE168093 BE087521 |
| | | AW603182 AW499660 AW499661 AW503473 AI902486 AW752292 BE174018 AW752296 AL041888 AW363953 AW605221 |
| | | AW605216 BE142816 AI435786 AA683040 AI907872 AW369725 AA380303 AA836230 AW419434 AW501816 AW502342 |
| | | AW502333 AW499671 AW502991 AW501843 BE168376 AW501486 BE245921 AW372603 AW372604 AW372601 AW372608 |
| | | AW372609 AW372612 AW372607 AW753096 AI902532 AI902594 AI902533 BE062377 AW811106 X97301 AA315458 |
| | | AL041463 AA287787 BE544417 BE311801 AW797378 AI940181 AA326261 AA326712 AW964943 AW751304 AW751303 |
| | | AI940199 AI940192 AI940189 AW751305 AI940167 AW176500 AI940202 AI940190 AI940201 AI940196 AI940203 AI940210 |
| | | AI940193 AI940177 AI940179 AI940204 AI940171 AW382746 AW382738 AW751312 AI940753 AW062939 AW803210 |
| | | BE175400 BE175388 BE094921 F28478 BE066456 AW372263 BE066575 BE066320 AW372262 BE066580 BE066455 |
| | | AW576937 AW392415 BE183395 AA381175 BE388469 BE066460 BE066315 AA377951 AA171585 AW605004 AW605007 |
| | | AW934766 AW934869 AW580606 AW605280 AW834728 R83817 AL121069 R20109 AA291361 AA524293 AW374653 H30144 |
| | | H44678 W24212 W06948 BE247374 BE180909 AA778442 AL041464 AA172255 AI962941 H26136 AA995288 AI921770 |
| | | AL040081 AI913019 AI829562 AI339531 AA837885 AI375317 AI762975 AW338852 AA938195 AI982701 AA682389 AW001982 |
| | | AW591316 AW173712 AW005552 AA039605 AW002560 AI559320 AW272286 AA045667 AI799670 AI803146 AI374801 |
| | | AI811885 BE326490 AW276312 BE049418 AI986113 AA286830 AI634266 AI198953 AI202521 AI380918 AA999947 AI492102 |
| | | R83376 AI337411 AI962358 AI635535 AW241544 AW003959 AI144276 AW576619 AI926506 AI889528 H43845 AW050926 |
| | | AI380249 R43161 AA581617 AI983302 BE465975 AI345956 AI341453 H26401 AW189035 BE350636 AI582500 AW316665 |
| | | AI439844 AI804761 AI470679 T32375 AI570375 AI273528 AW796804 AA658160 AA652859 AA503941 AA573555 AI358272 |
| | | AA573547 Z24817 AW576624 AA428059 AW023495 AI033000 N49249 AI969076 AA205445 AI636203 T30328 AI394724 |
| | | AA403302 AI968272 AI492974 AI582619 AI470826 AA894924 AI912962 AA569977 AI675362 AI473131 BE046814 AW300260 |
| | | T31879 AW168376 AI659621 AI810463 F35064 AI271700 AI699563 AA776537 T36184 AA565451 AI678338 AW438921 R16094 |
| | | N91497 AV662182 AI432919 AA910303 N91964 F18684 AA381902 AW364462 AI276689 AB015644 AW247214 BE047724 |
| | | AA347210 BE391395 H81815 N67777 BE208123 AW939117 AW939167 AW939108 AW939172 AW939123 AW939158 |
| | | AW605520 AW605509 AW605515 AW939122 AW939171 AA229095 AA229096 AA229232 BE382710 AI902544 M78278 |
| | | AW799890 AW889685 AW863018 |
| 103436 | 42952_1 | X98206 AA693431 AA244304 |
| 102806 | 8441_2 | AI246240 AF124733 NM_016358 U90306 BE407995 BE281254 AI566193 AI652266 AI671516 AI950190 |
| 103491 | 36109_1 | AF264750 AA904457 AW884708 BE183627 Y08478 AW962945 AA305849 Y08479 BE255205 BE258400 Y08468 AB040939 |
| | | T05961 AW807004 AW062952 AA135076 AW997220 AA090128 AW137099 AI640514 AW592676 H01987 AA134913 AA599219 |
| | | AA913906 AW573059 AA904722 AA904721 AA368411 W04955 H82698 AA077280 AW014128 AI191700 AI298476 AI540051 |
| | | H82435 AA774512 N53899 Y08482 AA332025 AW965281 AA331817 AW803125 AW992770 AA329962 AV659521 R78277 |
| | | R75636 AI681382 AL079859 AV659778 AA314956 AA127627 AA116095 AA121529 AI693624 BE612540 R21914 AA135986 |
| | | AI697706 AA234595 AI189138 AW055042 AI144389 AI263904 AI400175 AI275540 AW997221 AI742458 AI079937 AI138511 |
| | | AW196435 AW291117 N68458 H73663 AA116096 AA236552 AI983684 H73722 AA282356 AA932090 AA811462 AA737189 |
| | | AI636190 AW873132 AI468635 H01064 H13088 R22563 BE539692 AI581680 Y08470 AW836778 BE080692 AI091839 |
| | | AI572125 BE006050 BE006047 AW836837 AW501631 R09599 AW973566 AW263143 H61053 AA553507 AI214982 H02081 |
| | | AW997046 AW505183 BE143139 Y08836 Y08477 H73356 Y08465 H73223 N91302 AA641936 Y08464 Y08466 Y08483 Y08481 |
| | | AA506064 AW807922 |
| 126363 | 185896_1 | N94706 AW302202 N54724 W01777 AI949662 AA917338 N50068 AI857652 BE550711 AI340079 N63025 AA971513 AI467976 |
| | | AI694919 BE328279 AW206626 AI310369 AI307346 AA425773 AI862813 AA628674 AW475016 H10786 AI932791 AA244420 |
| | | AI567886 AA621126 AA244453 AA369087 AI678489 |
| 127008 | 175968_1 | AA223753 AA223879 |
| 127027 | 41793_1 | AL390088 BE207034 Z42964 BE242833 AW583731 BE616399 BE621126 BE272661 BE561537 AW328160 BE513636 |
| | | AW732368 AA367575 AA317809 AA325684 AL043346 AW997819 BE207681 AA442690 N33908 AA329967 AV661996 |
| | | AA364929 AA132857 R41876 W30718 AA340232 AA923628 BE618790 BE383719 BE384891 AW973886 AI680631 AL042633 |
| | | AL044048 T64995 AI814372 AA046927 AA707444 AI351042 AW157778 AW303835 AA559116 AW157476 AW440951 AI146410 |
| | | AI570583 AW473765 AA931145 AI654587 AI089717 AA132763 AI199778 AI144493 AW473769 AW087749 BE166742 AI089476 |
| | | AW083264 BE206437 BE222620 AW615413 AI356436 H98622 AI674887 AW583314 AI081960 AA133159 AI932568 AA641830 |
| | | AW245125 AA543056 AI289158 AI538987 N67489 AW836308 AI042462 AA639343 AA706144 AA653493 AI363727 AA724790 |
| | | AA596032 BE277184 AI126007 AI241020 AW627956 AW440961 N30676 AI806651 AA583594 AA732028 AA731516 AI439229 |
| | | AW995619 AA133204 AW248060 AI914553 AI689519 AI521043 AI566390 AI245111 AA594407 AI797417 AA131580 AA035240 |
| | | T33853 AI362604 AA535337 AI273040 R68453 AA043655 AI587360 AW572022 AA935227 AI697259 AA749181 BE263051 |
| | | R68454 BE560435 BE277826 Z49985 U30174 D61707 BE390825 BE618267 U30173 D61706 Z49984 BE268853 N99057 |
| | | AI874052 AA568135 R68452 H21498 AA043654 AA046984 AA362959 AA035318 R17261 AA131629 BE166750 BE298260 |
| | | N25142 |
| 127036 | 1150_2 | AI468598 AA232868 AL043752 AW881016 AW881015 AW881214 AW880761 AW881018 AA255767 |
| 103513 | 42559_1 | Y10209 AW341910 |
| 127041 | 1534405_1 | Z43067 F12549 F06090 R11929 T74454 R12925 |
| 126406 | 95703_1 | N76683 AA034096 AA034082 |
| 127064 | 1690305_1 | T84214 Z43709 R05654 |
| 133660 | 27207_1 | H14843 C14857 F06523 AA322683 AW964649 H83334 AW894647 AW894608 AW578353 BE007437 AW894648 AW894609 |
| | | AW896993 AW897085 AW581270 R36029 AL117536 AW896412 AW993019 AW859782 AW859772 AW376619 BE082963 |
| | | AW376687 H24066 AW847946 AA091541 D51576 D52043 D52146 D51628 D52155 D51675 D51777 D52101 D51602 D51843 |
| | | D51878 D51751 D51820 AA319632 AA351569 AW630840 BE073033 D51985 AW838560 AW838633 AW838631 AW838554 |
| | | AW838557 AW838559 AW608175 AW608194 AW838558 AW838562 AW838564 AW838571 AW608170 AI926544 AW608177 |
| | | AA296774 AW838624 AA318941 AA319199 AW960724 BE073078 AA773508 AI925495 AI889128 AI633207 BE221497 |
| | | AI126062 AI344458 AW608176 AW838627 AW194765 AW838630 H24379 T23982 R62391 BE620113 W02397 R87373 |
| | | AA364488 AW963466 AI696793 AA012998 N72571 C18550 AI298061 AI084648 D51984 C14379 D52154 T03606 AI499301 |
| | | AA017178 AA017170 BE077301 AI889566 AI002790 AI205010 AW104793 AA298374 AI049720 D51575 D52042 D51627 |
| | | AI281148 AW104111 AI142928 D51601 D51842 D51819 D53690 AI242974 AL049247 AL036365 AL036366 D45664 |
| 102934 | 16110_−11 | X13451 |
| 126479 | 1297811_1 | T78141 T75126 AW953462 AW956923 T08967 |
| 125877 | 18687_2 | AW969436 AK000755 AA176076 BE258269 |
| 103650 | 43833_1 | H16443 T66760 H11411 Z70220 F12208 T66258 AW950931 AA350846 H06005 F12617 N51174 T74154 R87613 T33928 |
| | | Z45881 AW961235 T31307 AW891440 H24171 R18227 R19098 W26891 |
| 127191 | 206872_1 | AA625286 AA297581 AA303053 AA303052 |
| 103682 | 41323_1 | AF275815 AA018302 AA000993 AA021290 H86017 R69197 AA056286 R88858 AI570404 H43774 AA059385 AA010839 |
| | | AA056237 AI436578 AI769179 AA019840 H86715 H84086 AA000994 H85881 AI762043 AI079265 AI738426 AI654986 AI738440 |
| | | AW197623 AA021232 AW161646 |
| 126568 | 163056_1 | AW749469 W68106 BE162986 AA190515 |
| 125925 | 1502610_1 | BE550996 H28737 |
| 125952 | 87085_1 | AA017723 H40709 |
| 125953 | 1582862_1 | H41682 H40829 |
| 104328 | 243947_1 | R11272 D81932 |
| 104340 | 46289_10 | AA426189 F15201 |
| 127231 | 221630_1 | AA317400 AA434584 |
| 126619 | 186375_1 | AA247746 Z28861 Z18915 |
| 126621 | 164483_1 | F01180 F22065 F21334 AA192638 Z28873 F31558 F33176 F30552 F35761 F18952 |
| 119404 | 115373_1 | AA302840 T93016 T92950 AA077551 |
| 127331 | 379388_1 | F20186 AA622352 |
| 127347 | 133106_1 | AI922293 AI123420 AA833906 AI417534 AA402715 AW275969 AA470879 AA402585 AA425800 W74482 AA410632 AW051466 |
| | | AW057511 AA443121 AA121406 F09029 F37501 AI909933 AI909928 AI909925 AI909932 AI909931 AI909926 W79080 |
| | | AW958666 AA312211 AA428350 AA410631 BE382380 BE394251 AA122083 F11368 F05224 |
| 127357 | 288073_1 | AA424107 AA452788 |
| 127359 | 16354_−4 | H72971 |
| 127378 | 299674_1 | AA446387 AA452696 |
| 126730 | 297653_1 | AA442429 T19477 |
| 126759 | 109907_1 | AA063089 AA679118 AA063642 |
| 126762 | 110350_1 | AA064613 AA064671 |
| 126767 | 327401_1 | AW968865 AA491199 C17148 |
| 112309 | 1576900_1 | R55021 H26613 |
| 128099 | 501940_1 | AA905327 AI148973 |
| 127452 | 327502_1 | AA504447 AA491317 |
| 126808 | 121207_1 | AA662559 AA086320 |
| 126844 | 133298_1 | AW881997 AA121637 AA299325 |
| 103985 | 19574_1 | AB032251 AW852071 AA279887 AW852124 AW129468 AI583544 AA704421 AW999851 BE081960 AW390494 AW582002 |
| | | AI862331 AA700432 AI186130 AI720707 AA236286 AI336961 AW015054 N91172 AI698141 W32818 AW896124 AI701519 |
| | | AW894048 H87416 AA234040 W17142 R84565 AI803033 AW867682 H27242 AA366224 AA357157 H86935 BE090270 |
| | | AW753846 AW362772 BE464864 AA704907 AA704948 AA704938 H27235 AI033427 AW850923 AA480900 AW502989 |
| | | AW851237 AW851374 BE252815 AA084307 AA313880 AW961200 AA480961 AF114105 AF114103 AA486042 AW375963 |
| | | AW752365 AW375959 AI290321 AA488659 AI242380 M85386 AI825492 AW499531 AW502114 AW502349 AI825312 H45002 |
| | | AA177096 AA248058 AA252844 BE327236 AW418631 W74679 AW582000 AW390459 AW609218 F05276 AA425478 R14173 |
| | | AW609219 AW868913 AW868854 AW868905 AA333683 H02382 T35574 D12400 C14910 AW894285 AW903587 N64611 |
| | | AW975302 AA541375 AI479002 AW006220 N51384 AI142472 AA659902 AA534253 D58103 H02279 W74710 H44955 |
| | | AA425278 N59267 H28589 AA804452 AI191286 AW197903 AI864639 AI122991 AA469450 H28584 AA252812 AA467779 |
| | | AW130113 AW872897 AA789290 AA935730 AA906959 AI990477 BE504778 D57881 AA721144 AI341486 AI638797 AA765502 |
| | | Z18803 |
| 126872 | 142696_1 | AW450979 AA136653 AA136656 AW419381 AA984358 AA492073 BE168945 AA809054 AW238038 BE011212 BE011359 |
| | | BE011367 BE011368 BE011362 BE011215 BE011365 BE011363 |
| 127553 | 202308_2 | AA505046 AW969109 AA505047 |
| 127568 | 479942_1 | AW892676 AA853877 D44747 |
| 126977 | 171173_1 | AA210697 AW962138 AA309665 AA210819 |
| 126982 | 171753_1 | AA211419 AA211566 |
| 113195 | 178688_1 | H83265 T63524 AA304359 AW960551 AI672874 AI749427 AA227777 AW027055 AA971834 T49644 T54122 AI983239 |
| | | AI808233 T91264 T96544 AI350945 AI709114 R72382 T48788 R48726 AW385418 AI095484 T49645 AA928653 AA570082 |
| | | AW007545 T57178 AA516413 AA913118 T57112 AA564433 AA774503 AA367671 T59757 |
| 128260 | 38164_1 | AF119848 AI132994 AA505575 AI307657 AW205687 AW291922 AI347642 AI308117 AA398902 AA741579 AW269612 R72857 |
| | | AW418818 T23611 AI016388 AA641630 AW009309 AI078310 AW263980 AI971867 Z32873 R00068 AW028860 R57097 |
| | | AA402507 AW965254 AA114126 AA331445 AA333324 |
| 128279 | 1561282_1 | H18818 H08885 |
| 113213 | 23798_1 | NM_001395 Y08302 AI434019 AI470328 AI261807 AW024965 AI806537 AI830549 AI640337 AI219065 AW271700 AW028488 |
| | | AI133339 AI859205 R51175 U87167 BE379324 BE392008 AA340819 AA343110 T57275 D59164 AW299312 AI434422 |
| | | AI936390 AW024975 R40262 AW269126 R09430 T56590 AI367247 AI253132 BE464248 T58658 AW207785 T58607 |
| 120461 | 22556_18 | AA810830 AA251301 AW962926 AW962922 AW962925 AA371510 AW817535 AW817579 AA251629 AW837817 |
| 127705 | 966283_2 | AJ003322 AJ003324 |
| 127787 | 458957_1 | AA809310 AW975709 |
| 112746 | 93768_1 | AA541537 AW009489 AW950203 F25552 AA834645 N56270 AA029543 AI633587 F30948 C04983 AI628613 AA885458 T49449 |
| | | F13651 AW004753 AA369573 AA356955 AA355112 T49448 AA918022 AI910358 H40428 AI292038 AI684655 AI244426 |
| | | AA522725 AA352551 AA909796 AA494329 AA873880 AW957305 AW955082 AA552169 AA974888 AA977363 R93237 |
| | | AI865725 AI201852 AI244721 H40429 AI204631 AA029544 AI015353 AI245274 AI191342 AA662656 AA829667 AW001837 |
| | | AA626833 AA585220 AA522987 AI339186 |
| 128410 | 288073_1 | AA424107 AA452788 |
| 128440 | 210591_1 | AW090340 R52194 T87269 R17095 T91445 N76763 R82785 AW512315 AA534961 AW002002 H41133 AI985800 AI278720 |
| | | AI762576 AI798477 AW081499 AA314880 AI918168 W92216 T91358 T79785 F04683 AI824664 T78491 AA302483 AW958008 |
| | | T85160 T87183 R11236 R16208 F01406 AW150480 AW627337 T79354 |
| 127854 | 443883_1 | AW976796 AA769520 |
| 120606 | 39874_1 | AF169690 F08337 AA282956 AA282955 |
| 120734 | 208882_1 | AA299948 AA299949 |
| 105897 | 8177_1 | H03573 AA310766 AL133603 AI700910 H00505 R82804 H60605 T10498 BE176241 N99617 BE537165 AA443413 H02733 |
| | | AI267539 AA094718 AI299771 N50199 R64087 AA709363 R26971 AI962859 H03899 H03888 R63010 AI346026 AA328951 |
| | | AI025251 H89260 AI472009 AW029442 AA211594 AI310418 AI913561 AW515607 AW593597 AI926843 AW073920 AW002745 |
| | | R62957 AI439422 AA401091 H58246 R82805 AI174264 AI590100 AW510481 R26739 H02624 |
| 100097 | 17246_13 | AF002224 AA017358 AL119785 AI379446 AI632616 |
| 106782 | 83684_1 | AW054886 R48313 AI200750 AI358095 H28124 AW613845 AA010033 AW182407 AA010034 AI986052 AI032851 AI719051 |
| | | AA478487 AW410484 R48418 |
| 114610 | 117244_1 | AA525225 AA525299 AA081079 |
| 114636 | 109698_1 | AA075488 AA129081 AA074851 AA082852 AA074732 AA084908 AA084751 AA076042 AA131172 AA085374 AA079519 |
| | | AA074510 AA113824 AA102437 AA070833 AA070143 AA084693 AA084389 AA076373 AA075492 AA062834 AA084335 |
| | | AA078829 AA079344 AA069916 AA079275 AA070914 AA654069 AA081976 AA080957 AA083115 AA070942 AA085296 |
| 107518 | 45315_1 | X60152 AI871483 AI871473 AI871251 AI631860 |
| 107521 | 44111_1 | X78262 AL037659 |
| 130420 | 23038_2 | NM_003105 U60975 Y08110 AW501858 AA984813 AL042811 C14671 D59983 AW996611 AW502726 AW408730 AL120016 |
| | | T07220 AA972515 BE169618 AA053753 N45548 AV655750 AA485180 AW080066 T51689 AA714788 AI089413 AA748187 |
| | | AA744864 AA977151 AW167224 AA465100 AI692884 AI800689 AI419483 AI915371 AI264290 AI919227 AA687175 AI498499 |
| | | AI089844 AA721674 AW291409 AI377801 AI049681 AA767458 AA053631 AW473092 AW079490 N48698 R38875 AW501274 |
| | | AI817699 AA847277 AA761843 T07221 AA873077 AA568215 T51538 AI474852 AW505117 N50751 |
| 100403 | 19804_1 | AA302974 AW952936 AA297841 T17453 AL040862 AL042305 R18514 Z45908 H09477 R51937 H02949 AW672666 AA339081 |
| | | AW024007 AI910124 R41507 R02276 W72600 AW606045 T30584 AI968122 AA532894 AA047712 AA024602 AI814501 |
| | | AI479333 H09478 AI801077 R63410 AI469769 R63363 AA731181 W67437 AA613000 AA125822 W60322 AI199934 AI032499 |
| | | AI673744 AW675418 AA594241 AA047679 AI148807 AA948543 AI805752 AI362640 AA883795 AI873413 AW292015 BE503009 |
| | | AA960821 AI302435 AA911051 AI192308 T31342 AW589705 T32492 AW384043 AI968407 BE501316 AA074832 AI611662 |
| | | AI969799 AI216685 AL117466 NM_016733 D85527 AW992053 Z44073 BE078508 R70709 AL046533 AA402079 AA125952 |
| | | AA337799 BE252284 BE253079 AA325230 C18518 W67436 T83901 AA353025 AA024601 BE068484 AA782927 AA401915 |
| | | AA369003 R53606 W76565 AL040863 W60241 AI967950 AI424849 AI582302 AI769611 AI394573 AI949294 AI656190 AI885933 |
| | | AA670336 BE465592 T17452 AI569505 AA075098 Z41532 T35104 AI699545 R02275 AA368470 |
| 100476 | 24092_2 | NM_001907 X71877 X71874 BE140276 AA748397 AA213801 AA830996 AA765114 AW874469 AA811623 AA844904 AI371956 |
| | | AW451919 |
| 108360 | 112653_1 | AA129037 AA071539 AA071460 AA129036 |
| 108390 | 112416_1 | AA121999 AA121985 AA071108 AA071116 AA071434 AA148869 AA075070 AA122000 AA121986 AA071117 AA071109 |
| | | AA148655 AA075232 |
| 108392 | 113549_1 | AA075124 AA075208 |
| 100534 | 20653_36 | T85231 AW161503 AW401856 BE397508 BE387085 BE514425 BE387606 BE391894 BE562022 BE561561 BE537755 |
| | | AA326129 BE397421 T49865 BE259375 BE562435 AA206146 BE272379 AA206012 BE252021 BE264415 BE559498 BE622499 |
| | | BE560425 AF141349 AF070600 BE260687 BE267430 BE273769 BE409463 BE378506 BE410826 BE540360 BE514334 |
| | | BE265991 BE259493 BE257982 BE541302 BE275305 BE249816 BE263407 AA306533 BE312339 BE537252 BE313173 |
| | | N44590 BE397957 BE314066 AF070593 AF070561 BE395620 BE539980 BE278635 BE622457 BE561276 BE561240 BE539757 |
| | | BE398025 BE539587 BE264804 BE312816 BE618175 BE312236 BE536880 BE275163 BE409064 BE268157 BE256878 |
| | | BE396565 AA094095 BE512689 BE539943 BE613640 BE561500 BE314652 BE560895 BE270780 BE394023 BE314771 |
| | | BE258043 BE545484 BE539560 AI524946 BE268922 BE313646 R21491 BE257007 J00314 T55776 BE514288 AA079759 |
| | | V00599 AA090885 BE268939 AI424151 BE393150 BE513223 BE311978 AA427899 Z36874 BE267470 BE270600 BE270542 |
| | | AA092574 BE513746 T56664 BE260686 BE561863 T80533 BE614348 AI003020 AA196886 BE395929 BE256732 T80512 |
| | | BE561248 H10806 N23553 AA090625 BE378633 BE299481 BE251134 F03598 BE158989 BE271056 BE271117 H39974 |
| | | BE537728 T95426 T93847 R15283 T82017 BE561756 BE254463 BE250916 AA522520 BE562294 BE561853 BE562013 |
| | | BE410876 R17504 BE254605 BE254904 BE315454 BE408837 BE256847 BE259444 BE265709 BE395378 AW869359 |
| | | BE019791 BE378840 BE270975 AW246214 AA352945 AA378509 BE275472 BE389534 AI609281 BE295688 AI972188 |
| | | BE388702 AW351598 BE617054 AI922439 BE258416 AL119346 AW351963 AW351971 BE388178 AI624652 AA780798 |
| | | BE268311 R35263 BE251613 AW366022 AW177828 AW057797 AW366021 AW177888 AW366065 BE140945 AA670139 |
| | | AA773017 BE539636 BE139520 BE270946 AW058238 AA191567 BE312975 AW089641 BE398147 BE268285 AW272598 |
| | | AL119339 AW079784 AI829482 AI982828 AI862019 AI816169 AI633046 N59068 AW731944 AA093100 H03237 AW575087 |
| | | BE301207 AW780181 BE141633 AW664164 AA626909 AW516663 AW872823 AA551910 AW571502 AI954158 AA548713 |
| | | AA599912 AA301057 AW574903 AA614152 AA613830 AW771103 AA706556 AI023390 AA551248 AA780901 AA633446 |
| | | AW419192 AA404660 W58436 AA523401 AA578734 AW615710 T65580 T57191 AA486211 AA604684 BE208800 AA669875 |
| | | AA622085 BE302083 BE263081 AA548072 AW156997 AI568868 AI627575 AA565217 AI870317 AI282474 AA704034 AI253598 |
| | | BE328271 AA564664 AA161128 AA604105 AA609087 AW103504 AA605238 AA704108 AI221585 AW574515 AW117994 |
| | | AA058521 AA604281 BE251293 BE017988 AI754462 AW245235 BE094031 AW862436 AW105529 BE544089 AW303918 |
| | | AW130826 AI721132 AA433888 AL038189 AW474816 AW182745 AA551345 AI445794 AA398452 AI224993 AI689213 |
| | | AW249709 H15542 AW196813 AA618551 AI445281 AI638121 AA807410 AA911530 AI334093 BE269844 AA847782 AW276513 |
| | | AW589524 T03418 AW170654 AW468976 AA570676 AA570715 H41474 AA010074 AA600283 AI755222 AI193239 AA774176 |
| | | AW770036 AI332603 AI129785 AI280055 AA666338 AW732127 AW028394 AA102641 AI920834 AA602712 H06697 AI095237 |
| | | AA401908 AW250765 AA631796 AA550854 BE260391 BE614352 AI690333 AA452272 AW575264 AA878265 AA190483 |
| | | AI832049 AA195830 BE439812 BE208085 AA826727 AI929438 H10785 AI520704 AI364235 AA605138 AA513116 AI250046 |
| | | BE263592 BE538689 AA171900 T03755 AW241170 AA565529 AA186883 AA872245 AI251930 BE538023 AA181759 AA187178 |
| | | H28109 AA115169 AA513309 BE545993 AA714730 AW897950 AA143782 T15562 T49475 AA196794 AA219637 AW574805 |
| | | R61459 BE294610 AA653641 AW246595 AA122178 AA079030 BE259347 AA125802 AA223509 AA071105 AA056515 |
| | | AA115965 H98828 AA975764 R44928 AA122173 AW499013 W68765 AI306457 H70716 AI866327 AA101102 AA523008 |
| | | AI567597 AA146744 R88666 AA197332 AI086825 AA197306 AA127186 AA648686 AI758939 H66791 AA158684 AI423641 |
| | | BE622207 H96899 AW748838 AI683391 T57141 AI360649 AA084328 AA205369 AA605035 AA730337 AA190782 AA622674 |
| | | H70715 AL035929 R53569 BE410471 AA826991 BE304945 T57181 AA190793 AI826886 R61544 AA357249 AA730680 |
| | | AA349636 AA598937 AW075466 W52142 AI270583 AA693372 D53633 T78875 AI874190 AI926336 AI744448 AA458786 |
| | | AW019919 AI903107 AA243188 AA127188 AA085973 R50969 AW674943 AW674933 N33158 T99016 AA386238 H05817 |
| | | AA227228 T93797 BE408530 AL035916 AA199872 AA196440 T61038 AI193541 T03458 H04813 T26581 AW074308 F28259 |
| | | AI420254 AW406597 AI752919 AA349540 AI961511 AI026726 T16511 T61589 AI872394 AW069694 AA350697 AI766468 |
| | | N84462 AA085828 AI049703 F09606 AW247712 T16319 T95336 AA427580 AA219549 N54075 BE383804 AI934238 T23636 |
| | | T23966 AI540707 AA852250 AA480844 F03543 AI682148 AW029145 AA350647 T57116 AA121780 AA362506 T89452 F03033 |
| | | BE544518 BE537982 AI400848 AW249805 R40175 BE547154 AW194658 BE304905 F03972 AI887116 AW662060 N64153 |
| | | AW090474 AA111861 BE544487 AW247971 AW189742 AA083676 AI689361 AI865447 AA205201 AA995389 T57070 AI749217 |
| | | AA620398 AI205275 BE278095 W25238 AW498851 AA361225 W24643 BE293452 T60878 BE378902 AW675067 AW419249 |
| | | BE546138 BE622685 H37989 BE298837 W02581 W03143 W03350 BE090146 D56472 AA477356 AA369046 BE617569 |
| | | AW405756 BE068241 H94917 R93689 BE536003 H78709 BE293724 BE207577 AA324949 AW406102 R66280 AW404427 |
| | | AW498515 AA283600 BE091536 N78168 N94034 H13896 H40383 AA426247 H38210 AW381001 AW381031 AW384779 |
| | | AI187246 AA356920 BE614174 AA361144 AA954817 AA373947 N51059 AA714393 AA361010 AA858368 AA128860 AA186577 |
| | | R97861 BE300605 BE617658 AW407835 AA007136 W04495 AA714521 W01702 AW675093 AI129627 W23528 BE622513 |
| | | D52760 AA580710 D54714 D54912 H39979 AI189140 AA604554 AA379346 AA379753 BE293739 AI247952 H19587 H23957 |
| | | H22624 AA022986 H80389 H80376 W32684 H74304 W73673 BE619989 H46803 AA326012 BE256750 BE293868 AI687073 |
| | | BE540791 AI554627 AI669399 AI052649 N74381 BE254994 W35343 R83888 AW405482 BE300603 AA190680 AW409945 |
| | | AI565178 Z25891 H95714 AI587135 AW881291 N74433 AW580801 AI126014 AW583313 AW385805 BE392285 AI002971 |
| | | R85251 AA928186 AW883103 AW577705 N47822 AW245647 AI186441 AA455487 AA876464 AA512933 R48210 AA167661 |
| | | AA715724 T56604 BE315352 R25751 BE163471 W77978 AA617822 AA618045 AW973348 AA641395 N32598 N58159 |
| | | AA604336 AA617894 AA748311 AA599921 AA617899 H94863 AA029390 AA706656 AA206124 AA778101 BE300604 T15728 |
| | | W57648 AW575696 AA029389 AA599861 AA640585 AA613863 AA526077 AA577614 AW575035 BE183827 AI709081 |
| | | BE183680 AA330412 AA580149 AW574766 AI609157 AA557458 AI801804 AW085646 AW339731 AA907669 AA489663 |
| | | AA486665 AW166938 AI500319 AA181843 AA605173 AA486566 W94458 AI623349 AA858195 AA558201 AI249662 AI092843 |
| | | AA905720 AI682690 AA773433 AA434385 AA022948 AI249681 AI619676 AI084530 AA554408 AI376770 W35304 AW574722 |
| | | AA279871 AA641309 W00970 AA564566 AA206053 AI682641 AI686270 AA216784 AI371057 BE208021 AI816060 AA977763 |
| | | AA988221 W56716 R66281 T60692 AI371108 AW316667 AW090548 AW263786 AI688536 BE300865 AW118162 AW089606 |
| | | AW157567 AW439435 AW673929 AW027607 AW474885 AI984259 AW571884 AI880916 AA758525 AA070410 AW190306 |
| | | AW246746 BE221650 AI630670 H28485 AW468627 AW651608 AA360204 AW513778 AI608753 BE302410 AI750057 |
| | | AW674261 AW510365 W94350 AA761293 AI869840 AA301790 AA933607 AA227144 AA716677 AA181762 R83889 AI079599 |
| | | AI000208 N99897 AA070466 AW245167 W68620 N83377 AI361698 AA464157 AA496711 AA205826 AI361699 H80288 R50934 |
| | | H98743 AA179708 D52483 AW249546 T49476 H80293 W73625 AA099853 H03238 AA179703 AI880803 AA653206 AA457733 |
| | | AA931701 AA190330 AA906004 H79483 AI084626 T57243 AA079760 T55734 AA507899 W73915 T74565 AA523607 N69544 |
| | | R97811 AA190385 R86252 AI359329 AA157496 AA070716 AW162385 AI929421 AW651618 AW087897 AW770466 AW073321 |
| | | AI813809 AA113799 AI342925 AI754395 W60153 AA665490 AA653483 AW087840 AW169380 AI754330 AA181774 AW243958 |
| | | R76858 AA936814 AA477111 H80279 AA496723 AA633000 AA243218 AW169688 H80274 R48211 AA743251 AI221630 |
| | | AW593584 H40328 W56761 H95640 R93010 AW250165 AA497004 AA279793 W20432 AA775006 AA401150 T34421 T29637 |
| | | AA678773 W23704 AA723093 AW073071 AI140379 AI188662 AA680074 AI081730 AW874489 AA226908 AI452689 AA531164 |
| | | AW498837 AA670295 AI061112 D20132 AI937246 AA580593 N70277 AW993170 AA205918 AA581651 AA129695 AA548261 |
| | | AW673898 AI000118 C13991 BE247777 AA653439 AI198072 R26594 AA282708 C14467 C14444 AI000629 C14510 AA775369 |
| | | AA885560 F30063 D51934 D51834 D51478 D51931 D52011 D51918 D52160 D52045 D51888 AW245330 D52030 AA936639 |
| | | D51494 D52144 D51448 D52138 D51730 AI419648 D52007 AW021403 AA935494 AA935487 AW770291 AI702431 AA687083 |
| | | AA362527 D51696 R29267 AA653197 AA888552 D54444 C15448 AA478045 AI589903 D55942 H40340 AI864969 AI341782 |
| | | AA353097 AW439647 AW305322 N54940 AA935976 AW248042 AI783806 BE349217 AA969923 AA383726 AA357323 |
| | | AA007135 AI915436 N71185 AI123662 AA328354 AA331419 N21981 T03306 D54551 AA983808 D54942 D55074 AA113880 |
| | | D52740 AW020376 AW020367 AA936029 AA555005 AA969180 AW812036 BE544746 N89212 |
| 100544 | 22955_11 | M55405 AW752552 |
| 100545 | 22955_11 | M55405 AW752552 |
| 124087 | 1561179_1 | H08773 R37687 |
| 100560 | 30266_1 | Z99916 U71216 NM_004076 X15144 X15145 X15146 AA652300 |
| 100571 | 7592_1 | L14561 AW496834 AW059849 Z99413 T29666 R14900 R93603 R51525 S49852 U15687 U15686 AI033177 W94597 AI056839 |
| | | R02057 AA134349 F05567 H73767 BE326691 AI984023 AI695688 AI750184 AI434336 AW029158 AI440089 BE046108 R51526 |
| | | R53074 AA025218 AW083388 R93604 AA732638 H47642 AI859270 R41222 AA593057 AA760815 H43399 N29456 AI337321 |
| | | H98205 AI752749 AA442913 AW961129 AL134557 AW439527 AI341304 AW190061 AW613371 AW439931 AA806627 |
| | | AA781883 W05800 AA731599 AI863791 AI370277 Z38623 AI470448 H15877 R53656 D82513 D82467 D82386 D82515 R15295 |
| | | AA096386 AW892808 AA300686 AW954282 AW959453 H29807 R25259 N46722 N46679 D60314 AA736829 Z99414 AI076802 |
| | | AW576457 H16400 AA933075 AI752748 N75064 AW873040 N39235 AI310710 BE328592 N39576 AI672714 AA747547 |
| | | AW769967 AI625462 AW169755 AW615361 R53545 AI758375 R46612 AW273091 AA736697 H29808 R42200 D14108 |
| | | AI783809 AI270395 AA952981 AA746745 R37023 C75654 Z39578 C75656 AW956278 AA338702 AA338701 AA279433 W58753 |
| | | AA041264 R02056 AW842224 AI215415 AW951238 AW022139 AA658234 AL119699 BE537020 AI795941 AA927068 AI765979 |
| | | AV655427 AA041498 AI436090 AI190267 AA397563 H43344 AI718564 AW192067 BE328781 AW768573 AI358433 AW631373 |
| | | AA262804 AW235152 AI570253 AA934641 AA776029 AW182028 AA279606 AV657690 AV657727 AV657350 N41480 AI926218 |
| | | T29285 BE143058 N72769 AA385114 |
| 100572 | 13049_1 | M60495 M60499 M60500 M24355 M62201 BE389102 M96943 L01090 |
| 100582 | 26433_2 | D13897 AI183955 NM_004160 D13899 L25648 AW136574 AI654355 AA913288 |
| 108409 | 113869_1 | AA075631 AA075578 |
| 100627 | tigr_HT2798 | Z25424 |
| 123526 | genbank_AA608657 | AA608657 |
| 100663 | tigr_HT3059 | M20022 M32505 M32506 M32507 M32508 X64880 X87678 X87680 |
| 100684 | tigr_HT3283 | S66933 U15688 |
| 100687 | tigr_HT3291 | L18862 |
| 100695 | tigr_HT315 | M31126 M38243 M74197 M94890 U04323 U08196 U08197 |
| 116389 | genbank_AA599011 | AA599011 |
| 100702 | tigr_HT3413 | L27065 |
| 100756 | tigr_HT3768 | M88357 |
| 100809 | tigr_HT4261 | L33990 |
| 100810 | tigr_HT4262 | L33994 |
| 100854 | tigr_HT4464 | U08607 |
| 102185 | entrez_U20230 | U20230 |
| 125090 | genbank_T91518 | T91518 |
| 100961 | entrez_J00148 | J00148 |
| 102254 | entrez_U28131 | U28131 |
| 125145 | NOT_FOUND_entrez_W38001 | W38001 |
| 125153 | NOT_FOUND_entrez_W38294 | W38294 |
| 116797 | genbank_H40486 | H40486 |
| 102354 | entrez_U38268 | U38268 |
| 102474 | entrez_U49973 | U49973 |
| 116902 | genbank_H70739 | H70739 |
| 116905 | genbank_H71420 | H71420 |
| 102643 | entrez_U67849 | U67849 |
| 125576 | 205199_1 | R21635 R66208 |
| 118579 | genbank_N68905 | N68905 |
| 120256 | genbank_AA169801 | AA169801 |
| 120274 | genbank_AA177051 | AA177051 |
| 113149 | genbank_T51588 | T51588 |
| 127759 | 808057_1 | AI369384 AA719568 |
| 113518 | 5697_40 | AW367788 AI093217 AA533602 AI300430 AI860318 AI869201 AA933015 AA077695 AA649045 AI016319 AW084216 AI969650 |
| | | AW169170 AI440341 AI744376 AA905125 AA194528 AA971323 AW510721 AA194483 AA076744 AW182167 AI955513 |
| | | AA578875 T89731 T89829 AA828731 |
| 113608 | genbank_T93113 | T93113 |
| 101046 | entrez_K01160 | K01160 |
| 122731 | genbank_AA457549 | AA457549 |
| 108316 | genbank_AA070160 | AA070160 |
| 108328 | genbank_AA070204 | AA070204 |
| 108394 | 482708_1 | AA642953 AA075144 AA084113 AA070130 |
| 108395 | 482708_1 | AA642953 AA075144 AA084113 AA070130 |
| 108417 | 483241_1 | AA070853 AA075749 AA075716 |
| 108436 | genbank_AA078801 | AA078801 |
| 108491 | genbank_AA082973 | AA082973 |
| 108499 | genbank_AA083103 | AA083103 |
| 122922 | genbank_AA476268 | AA476268 |
| 122938 | genbank_AA477119 | AA477119 |
| 122948 | genbank_AA477483 | AA477483 |
| 117046 | genbank_H89505 | H89505 |
| 101427 | entrez_M19508 | M19508 |
| 101559 | entrez_M32053 | M32053 |
| 117437 | genbank_N27645 | N27645 |
| 101798 | entrez_M85220 | M85220 |
| 117590 | genbank_N34904 | N34904 |
| 110349 | genbank_H40988 | H40988 |
| 101909 | entrez_S69265 | S69265 |
| 103392 | entrez_X94563 | X94563 |
| 119053 | genbank_R11501 | R11501 |
| 103457 | entrez_X99728 | X99728 |
| 119174 | genbank_R71234 | R71234 |
| 119229 | genbank_T03229 | T03229 |
| 103654 | entrez_Z70759 | Z70759 |
| 103679 | entrez_Z86000 | Z86000 |
| 119329 | genbank_T51832 | T51832 |
| 119343 | genbank_T62873 | T62873 |
| 119347 | genbank_T64349 | T64349 |
| 119523 | NOT_FOUND_entrez_W38041 | W38041 |
| 119526 | NOT_FOUND_entrez_W38049 | W38049 |
| 119529 | NOT_FOUND_entrez_W38053 | W38053 |
| 119564 | NOT_FOUND_entrez_W38206 | W38206 |
| 119566 | NOT_FOUND_entrez_W38209 | W38209 |
| 126908 | 533102_1 | AA180024 AA169866 |
| 119906 | genbank_W85818 | W85818 |
| 123022 | genbank_AA480909 | AA480909 |
| 114604 | 485245_1 | AA075966 AA076128 AA128755 AA128339 |
| 114666 | genbank_AA112274 | AA112274 |
| 100221 | entrez_D28383 | D28383 |
| 123143 | genbank_AA487595 | AA487595 |
| 114718 | 480609_1 | AA080912 AA075318 AA083403 AA078992 AA076594 AA062835 AA084926 AA081881 AA082953 AA113913 AA083821 |
| | | AA070343 AA134801 AA113892 AA075419 AA063293 AA071252 AA078900 AA113888 AA071406 AA075072 AA100879 |
| | | AA112153 AA062836 AA113865 AA074554 AA643251 AA084110 AA078783 AA084346 AA078993 AA079022 AA079776 |
| | | AA070181 AA085031 AA082954 AA082971 AA131328 AA063179 AA083366 |
| 100478 | tigr_HT1067 | M22406 |
| 100547 | tigr_HT2219 | M57417 |
| 100563 | tigr_HT2324 | Z11585 |
| 100564 | tigr_HT2324 | Z11585 |
| 123473 | genbank_AA599143 | AA599143 |
| 123490 | genbank_AA599723 | AA599723 |
| 322024 | 43541_1 | AA334384 AL137436 AA352089 AW341247 BE327629 AI669284 AI274156 AI123562 |
| 322033 | 33332_1 | AL137507 AW879790 AA349971 AI624899 AW294742 AW271762 T06484 AA788895 R35098 |
| 314251 | 194954_1 | BE011657 AA713589 AI440513 AA278620 |
| 320825 | 29807_1 | NM_004751 AF102542 AW380893 AF038650 BE304708 AW360892 AW360931 AW842622 AA307800 BE292814 |
| | | AW582119 AW582122 AW374998 AW374874 AI587061 AA583339 AW662377 AW192901 AW887756 AW887761 AI955582 AI150400 AA568218 |
| | | AA583146 AI832775 AA294856 AI445680 |
| 320832 | 170589_1 | AA214584 D56572 D57294 AA207006 AA210987 AA601292 |
| 320882 | 41486_1 | AK000655 AW024515 AI499162 AI673527 AA903900 AW084419 AI219549 D60890 AW243134 AI832098 AI811630 BE348831 |
| | | AI022831 AI475320 |
| 322128 | 46785_2 | R95860 AF085903 R95859 AI346033 |
| 322135 | 46801_1 | AF075082 H48639 |
| 322141 | 46810_1 | AF075092 H53478 H53853 |
| 322152 | 78409_4 | BE614761 AA263136 W00335 W00327 |
| 321525 | 27440_1 | AJ006077 AA377082 H78875 AA263148 AW958548 AA381830 AA381631 AW748909 H47990 AW402973 R31722 R82733 |
| | | AW195851 AI650977 AI635744 AW136376 AI913496 AI569147 AW263050 AW075972 AI538882 AI762430 AI924045 AW613027 |
| | | R74454 AI638560 AI476181 AA873030 AA936769 AI862049 AI955522 H00589 R31680 C20629 AI799176 AA835126 AA310578 |
| | | H13126 AI858430 AA377081 BE244193 AF288208 W26453 AV659072 AF288209 BE622910 |
| 320913 | 24943_3 | AA663733 D62598 D62472 |
| 321583 | 87512_1 | AA018518 AA059305 |
| 320957 | 39063_1 | AF151534 AI929478 W40378 H17557 AW163309 BE074271 AW854041 AW604408 AW853903 AA206576 AA133144 |
| | | AW854378 BE271915 F08002 AW293257 R58383 W95032 AI022248 H18001 R19681 F11341 AA778166 AI435068 AA702525 |
| | | AW176505 AA318946 AA705230 AW136167 AA704931 AW157512 W93328 AI694445 AW157539 AI301999 AA975516 |
| | | AA722698 AI569683 AI674496 H17453 AI910812 AI598186 D53709 N80796 AA285026 AA704939 AA159340 AA565703 |
| | | BE464932 AA477953 AA402072 AA476988 AI929436 AA133096 AI912377 H61238 AI457179 H18002 BE047110 H56139 |
| | | AA633441 AI500607 AA401959 H55753 AI767655 N93886 AA402908 H78194 D80936 R44236 H61239 BE551612 AI638233 |
| | | AI953226 AI744095 F09000 Z41080 AI700837 AW768854 AI872712 AA283182 BE383452 |
| 320994 | 9353_4 | BE618027 H22381 AA326465 AA326466 AW962215 BE207339 |
| 322209 | 46967_1 | H89360 AF086037 H89546 |
| 322224 | 47029_1 | AF086064 AA013034 N50099 AW903279 |
| 315021 | 344728_1 | AA533447 AF158241 AI240598 |
| 322264 | 47231_1 | AF086242 W67679 |
| 322265 | 47233_1 | AF086244 W68829 AI539008 W68737 AI685168 AI351133 AI633948 |
| 321632 | 286374_1 | AW812795 AA419617 H87827 AW299775 AW382168 AW382133 BE171659 AW392392 BE171641 AA541393 |
| 314429 | 238740_1 | BE146577 AA339680 AA972551 BE146576 |
| 306624 | 25108_1 | AA329384 S49006 AW405735 AW404287 AW405848 AW405979 BE538908 H70947 AI814190 AA381896 AW582731 |
| | | AW975580 T94914 AW474500 AW869616 AW974125 AW946246 H44560 AW802184 AI538108 AW609912 AW865715 |
| | | BE007659 AA443571 AW376161 AW797783 AA713566 AW577973 AA745472 AW270172 AA910107 AA713717 AW364122 |
| | | AA533898 AI523353 AA715899 H44507 AW947263 AW390753 T89943 R66359 H44320 T64100 AA715879 R54661 AW579645 |
| | | H68597 AI499528 AI699010 AW605041 AW605043 AA236700 AI290767 H27472 AW079688 T69967 T87728 AW869468 |
| | | AW873389 AI250670 AA617786 AI566627 AW627605 AW302334 AA617836 AW793186 H16015 R83107 T85409 AA643479 |
| | | AA582449 AW794654 AI819995 AW865610 AW380149 AW793863 AI281629 T64021 AA501599 R49882 AA565066 R69406 |
| | | N23918 T59642 R54662 AW973199 R53006 AA401087 AA496563 AW934874 BE171294 AW793133 AI581595 AW605835 |
| | | AW605040 AW605055 AW605033 AW605058 AA291844 AW382472 AW605005 AA421881 AA513106 AA744190 AW386065 |
| | | AW613785 AW605829 AA580905 AW605027 AW364134 AA464027 BE012096 BE008565 AA809035 AA809154 AA911395 |
| | | AA713636 AA745324 AA826230 AW368498 AV646747 T89967 H61164 AI540661 AA721571 C02031 AA523762 AA523746 |
| | | AI287256 AW841164 AA962139 AW797014 AA515246 AW364140 H45842 T94267 T72111 R49771 T70151 H42664 R83656 |
| | | R83590 AW380297 AW380263 X95750 T57717 AW391436 AA293355 AW380185 X95749 X95748 X95747 AW796510 |
| | | AA318628 AI001043 AA744404 AA745567 AA650437 AA715526 AW392762 AA516228 |
| 306644 | 34348_2 | BE263222 BE312446 AI002913 |
| 306669 | 7570_1 | AI340462 AI583268 AA079086 AI950777 AI301866 AI925108 AW876954 AW877000 AA525418 AA888549 AI934220 AW380220 |
| | | AA804858 AI927576 T61151 AW384053 BE391691 AA533856 AA248400 T48202 N57156 R68346 R26020 AL050332 W30806 |
| | | H61369 AA092592 AA230324 BE271217 AW372903 T48772 AA358002 AA094302 AA559856 AW373308 AW373315 AW373297 |
| | | AW373311 AW373314 AW373309 AW877055 AW770140 AW379805 AI581609 AW364144 AA078921 AA715432 AA654210 |
| | | AI004899 AA602209 W47464 AA506588 R26822 AU076528 AI535743 AI535704 AI535681 |
| 322302 | 47349_1 | W76021 AF088052 W72465 |
| 322309 | 47372_1 | W76622 AF086372 W72660 |
| 322337 | 47496_1 | AA249804 AF086490 W93549 |
| 322340 | 47509_1 | AF088076 W95222 W92523 |
| 322362 | 36526_1 | AF039697 AI860821 AW406650 AW515052 R49969 AA834501 AI951401 AA761660 |
| 321708 | 64670_1 | AA126365 AA055269 AI768638 N46910 BE047017 AA488423 AW021548 AW612323 AW007669 AI333685 AI633142 AI638022 |
| | | AI765587 AI151494 AA443764 AI373599 AI140433 AW295946 AA088183 AI811417 AI277748 AL079978 AI341834 AI144434 |
| | | AA055164 AI765262 AI140147 AI160673 AI686050 AA130038 AI699503 AI580411 AI276910 AW296875 AI927239 AW470427 |
| | | AI174424 AI675518 N20396 AI308224 BE221055 AI926097 H89681 H90776 AW023381 AA126261 AA025391 AA347308 |
| | | AA476817 N25488 AA442811 AW297971 AA025454 D81551 D81214 D61126 C15599 AA130037 AA088653 AV654662 |
| 321763 | 166644_1 | AA195602 W01148 N40632 |
| 313913 | 653504_2 | AW391342 AI813778 AW190911 AI963486 BE049275 AI922735 AI935914 AI263291 AI679178 AW192178 AW338885 |
| | | AW473496 |
| 306710 | 52443_1 | BE564350 AI024221 |
| 313960 | 130872_1 | AA113301 AA130859 AI805438 AA846950 AA130915 AW862833 |
| 306773 | 563488_1 | AI040750 BE062532 |
| 323014 | 140817_1 | AA305198 AW962351 AA134366 AA259244 |
| 315276 | 383168_1 | AA860090 AI961138 AW592114 AW087700 AI798907 AW340674 |
| 321847 | 45225_1 | T08401 Z83934 T16897 |
| 308069 | 33294_5 | AI470895 BE386951 BE391680 BE277351 BE385202 BE300283 BE299619 BE254885 BE391698 T17015 AL048342 BE387417 |
| | | BE315548 AA345769 AA374932 AA304724 BE277135 T31443 BE387617 AW582877 T33057 AA054826 AA305115 BE378981 |
| | | BE615689 W07028 BE537075 BE615271 BE252353 BE563255 BE391752 BE545875 AW580149 AA747256 AW276731 N85015 |
| | | AA299609 AA365061 AA302265 AW951660 BE538115 AW404883 AW583622 AA374519 AA225917 AA037286 W24285 |
| | | BE255801 BE254061 BE278256 AA377959 AA319628 AA316739 AA378914 W24421 AA658882 N87533 AA526881 AA376559 |
| | | AA378175 AA300193 AA330503 W69498 C03856 N85298 AW957966 C02647 H19560 BE616753 BE263169 AW250280 |
| | | AW162197 AW163162 AA301672 AW249055 BE616277 AA225272 AI557298 AW276939 AA327042 N83648 AW404841 |
| | | AI903202 AA314875 AI546852 BE616559 BE262330 AA385891 BE182717 AI557551 AA082310 AA354394 AI541339 AA327616 |
| | | AA344981 D53311 AA363102 AA171801 AA089673 AW162085 AA379891 BE277324 AA157651 AA304574 W52799 AA186652 |
| | | AW328567 AA187480 BE386732 AA369717 AI815603 AA303651 AW160407 AA056711 AA082790 AA329861 T39426 |
| | | AA650419 AI268280 AA471236 AA308712 AW733012 BE256165 AA745013 W06997 W21418 AA531583 AA308694 AI133371 |
| | | AA324238 W24570 N88175 W45648 AL022721 AA378455 AA378168 BE393810 AA317384 W21121 AA371342 N31367 |
| | | AA359623 NM_007104 U12404 BE388017 AA374579 AA380618 AA334401 AI557552 AA337150 AW732322 AA327632 |
| | | BE263809 AA657772 D30933 AW663232 AA315817 AA314480 AA370766 AA317760 AA315084 BE383638 AW327814 |
| | | BE302619 W21304 BE390854 AA373219 R94330 H84395 AA377636 BE252526 AW732866 AA299524 AW328563 AW674382 |
| | | W38535 W06840 AA332645 AA355580 BE258503 BE256923 BE271400 AA380757 AA385628 BE256989 AA332349 AA314985 |
| | | AA186515 AA305031 AA357482 AA316998 AA379264 BE390621 BE255056 AI207414 BE252354 BE254152 BE254578 |
| | | BE252547 BE266591 BE257651 BE294792 BE277990 BE391235 C15642 BE391151 AA301280 AA384750 BE273056 |
| | | BE312333 BE296962 AA314715 BE256346 BE313459 BE269894 AA310988 BE300029 AA157523 BE250757 AA545754 |
| | | AA083546 AA315252 AA224003 AV655919 AA315169 AA310685 AA301191 W19623 BE392967 AA303515 AA379649 |
| | | AW327286 BE560669 AW580113 AA352149 AA360956 AA316107 BE563100 N31353 AW675322 AA379584 BE019308 |
| | | AW583963 W37951 BE295910 BE251482 BE385588 BE270412 BE378750 BE396426 BE295210 AA336973 BE314395 |
| | | AA354788 BE295227 D55985 BE615081 BE260566 AA320506 BE278254 AA285366 AA303825 BE275375 BE313367 |
| | | BE260703 BE279256 BE382903 BE315113 BE313341 BE260804 BE620531 BE615973 BE266048 AA093478 BE616196 |
| | | AA360276 BE253746 AA314344 AA315622 BE386557 BE313445 BE253355 BE561084 BE296088 BE531299 BE255860 |
| | | BE408279 BE273842 BE378885 BE255055 BE271521 BE563765 BE252792 BE391016 BE258218 BE273725 BE273968 |
| | | BE272901 BE313660 BE304527 BE257956 BE254227 BE275320 BE253909 BE407614 BE614755 BE378776 BE613909 |
| | | BE407708 BE395973 BE513022 BE157875 BE615399 BE252775 BE279463 BE257109 BE271307 BE256275 BE276484 |
| | | BE257302 BE384605 BE258461 BE546662 BE409191 BE280351 BE252294 AA285364 BE378324 BE264740 BE292822 |
| | | BE620927 BE296544 BE271710 BE257492 BE252712 BE395931 BE266143 BE255921 BE255720 BE300136 BE531292 |
| | | BE391204 BE409180 AA320604 BE295038 BE390679 AF075335 BE315084 BE394350 W04926 AI110836 BE615235 BE295593 |
| | | BE275680 BE409184 BE253595 BE294473 BE253714 BE512867 BE295544 BE295554 AA296596 BE255510 BE378700 |
| | | BE513852 BE512932 BE257734 R31451 BE293044 AA379511 BE261311 BE397180 BE278214 BE407490 D56163 BE563686 |
| | | AA070681 BE255940 AA348975 BE253849 BE390468 BE396140 AA186986 W20120 BE389750 AA307189 BE378407 |
| | | AA125980 BE407415 BE276230 AA304996 BE389228 BE512779 BE617120 BE512670 BE272223 AW407102 AA370834 |
| | | H21183 W07800 BE562889 W05304 H02179 AA327191 H18595 H22622 BE257450 W46597 BE262207 AA002219 AW405652 |
| | | AA334037 BE294871 AW606479 AW384255 AA085885 AI903205 AW405432 BE298839 BE292956 BE262232 AA363253 |
| | | BE276322 AA300909 BE389123 AI909563 BE407174 AI535831 BE260382 BE382583 AA532637 AA380091 AI114568 |
| | | AA193129 AA192984 BE384600 BE407214 BE182724 BE539061 AW733001 AW406350 AA374881 BE384674 W87442 |
| | | BE276668 BE299430 BE274869 BE378528 BE396131 AW239494 AI630538 AA053478 BE280557 AA218746 AA299674 |
| | | AW406770 W07830 AA808817 AA146904 BE272999 AW408211 AW580062 AW606478 W07669 AW580120 AA379387 |
| | | AW580130 AA101207 BE394409 AA362957 AW392453 AA101121 W03827 AA166722 AI909564 R30769 AW580069 T58944 |
| | | BE261760 BE313004 BE312309 BE260766 BE314007 AA724391 BE538878 W04777 AA657588 BE259359 AA631041 |
| | | AA070065 |
| 314661 | 296144_1 | AA578229 AA436432 AA481375 AA481363 |
| 307474 | 25106_2 | AB037864 AI223793 AI916099 AI332699 AI264023 AW962587 AA412310 R37910 R37827 AI191724 N22920 AI916409 |
| | | AA932668 AA782068 AA865648 AW467014 |
| 323115 | 155930_1 | AI921875 AA170835 AA866613 |
| 322505 | 38889_1 | AF147315 AW173079 T53029 |
| 323170 | 980026_1 | U83527 AL120938 U83522 |
| 322518 | 38914_1 | AI133446 T50819 AF147343 T50665 |
| 323183 | 3032_1 | X73874 NM_002637 F00757 BE172051 BE081451 AW891243 W20171 BE619896 AA927528 AW374434 N90546 F01171 |
| | | AI681314 AI621206 AI366791 T27589 AW960206 AW393850 AW892627 AW892626 AW833974 AW833971 AA194696 |
| | | AW274557 AI813660 |
| 323185 | 18915_3 | R52177 AL121177 AW749837 AA354099 AA318422 AA318513 AA318429 AW961486 AW964802 AW961475 F12011 R12085 |
| | | R12113 R20096 AW836716 W87916 F12265 T64809 R96961 AA340929 R88075 F11199 H11127 W68153 BE465310 W67653 |
| | | AA777659 AA630420 AI423300 AI744317 AI681159 N81151 AI758899 AI991528 AI640256 AI361299 AA039678 R52083 |
| | | AI161215 AI240851 AA806482 AI159875 AW452889 AA987856 AI581408 AA746962 AI277132 AW802982 AI567611 AA761163 |
| | | AI277294 AW244127 H11684 AI381224 AI277139 AW952835 AI480237 AA188967 AA041517 AI798651 AA906163 AI081406 |
| | | AI032939 R41802 H95399 AW023038 AA890250 T65065 AA340673 F09658 AI248293 H11039 AA340628 AI955106 F11007 |
| | | AA028166 H98922 AA810707 AI769491 N21690 AI670911 AW005066 AI692255 AA028093 AA748907 AI919266 H11597 |
| | | H95373 R36867 R36878 AI222427 T23945 |
| 315322 | 444861_1 | AA770599 AI215099 AA992267 |
| 321920 | 1023421_1 | AW089866 N63915 |
| 321966 | 30334_1 | AL122111 M79226 AI656413 BE502197 F32670 |
| 321986 | 22722_2 | AL133656 AW269409 AI138865 AA954306 |
| 323243 | 140566_2 | W47525 AA134047 BE391212 AA330333 AA376355 BE304871 BE167342 H87402 AA631722 W45724 AA715517 AI925438 |
| | | AI804849 AW241617 AW403807 AI653435 AA134048 AW747874 AI922327 AI814967 AI935895 AA228865 AW504076 |
| | | AA225008 AW673858 C03914 |
| 316041 | 417912_1 | AA719183 AA736442 AA709125 |
| 322644 | 82848_1 | AW841642 AW747992 AW748000 AA007413 AA568664 AA779192 AW188983 AI471878 |
| 322649 | 24892_1 | F01266 NM_016549 AF239742 BE219554 AA902371 AI298989 AA533735 AA599216 AW025283 AI336116 N75315 AA621418 |
| | | AI671706 AI373650 NM_014556 AF216184 AI269271 AA780274 AA772950 AA932592 AA496722 AI186107 AA009748 |
| | | AA477144 AA788812 BE384172 AA009407 AA526549 D25270 AW882122 AW972929 BE174659 |
| 322682 | 37364_1 | AF063553 AI110679 AF090893 |
| 307625 | 10562_13 | AA768239 T98699 AW207784 AA343876 AW963055 AI299617 |
| 314893 | 3084_2 | AW969114 AA761093 AW082191 AA505065 AW087800 AI719655 AA576454 AW375968 AI804392 |
| 323333 | 62251_1 | AV651680 AA228883 AA367341 AW962458 AA628024 AW172426 AI767785 AA313012 AW963323 |
| 300470 | 59496_1 | T87841 AA233726 AV648314 BE545191 AW969738 AA579641 AA516077 AA483490 BE612779 AA460132 AA659710 AI149891 |
| | | AA113277 AI889233 AI028588 AA461487 AA074311 AA682750 AI421205 AA985248 AI753378 AI914588 AI493749 AA936668 |
| | | AA633392 AA722409 AI424537 AW609606 AW815447 AW268600 AW609594 AW601214 AW082930 AI752615 BE550576 |
| | | AI989665 AA046575 AI201331 AA169112 AA169113 AV648431 AV648389 |
| 322738 | 26089_1 | AF200496 AI343258 AI014548 |
| 308337 | 3328_20 | J00124 AA158848 AW452899 NM_000526 AW384139 BE140708 BE140690 H28274 BE140404 AW844922 BE157342 |
| | | AW580043 AW384049 BE157333 BE157179 H44127 BE140671 BE614947 AW380727 BE184532 AA808075 R73201 |
| | | AW949948 AW950242 BE183306 BE183297 AA587174 BE183243 AI591112 AI609229 AI590690 AW606369 AI609238 |
| | | AI608848 AI590885 AW084944 AI609220 AI609210 AA583890 AI609274 AI609226 AI608878 AA550914 AW170430 AI608947 |
| | | AI608606 R88146 M28646 AA587248 AA583985 AW872988 AA583993 AA586980 AI284481 AA584051 AA587024 AW800015 |
| | | AA587313 AI283394 AI143314 AA595434 AA583917 AA922332 AA584021 AA583576 AA862004 AW873168 AW796060 |
| | | AA862049 AW873161 AW079705 AI366769 AA583724 AA583889 D29065 AA113288 AW083705 AA584334 AA586711 |
| | | AA583558 AW117878 R72295 AA583842 AW996598 R73138 H28224 R88223 AA586985 AW369022 AA602252 AA641063 |
| | | R72845 AA861952 AA587149 AA587308 AW369033 AA584344 AW799955 AA583732 AW368990 AA862083 D29595 R53012 |
| | | AA587254 D29462 AA158849 R72649 BE181541 D29604 AI127631 D29297 H44051 AA583575 AA586762 D29122 AA583878 |
| | | AA595946 AW368880 AA587318 AW368881 |
| 322774 | 104619_1 | AA131111 AA132859 AA053057 |
| 314938 | 337806_1 | AA515635 AA516338 AI801993 |
| 307783 | 697809_1 | AI347274 AW844024 |
| 301119 | 33384_1 | BE621320 BE266806 BE276582 AW516729 AF142579 AW451687 AK000069 AA325236 BE168997 W73105 AA715365 |
| | | BE278873 AA808894 AA386371 AW517942 AW750993 BE140314 BE392384 BE621757 AA318192 BE548173 AW152607 |
| | | AW166898 AA352215 AW841506 T59802 AF147378 AA335719 AW956069 T59668 AA826362 AI961329 AI290469 AW197375 |
| | | AI805651 AA160748 AA581089 AI968889 AA581100 AA501478 AI621069 AA468534 AA503715 AA658457 AI144504 BE387827 |
| | | AA159880 |
| 301163 | 427639_1 | AA732066 AA807070 |
| 324031 | 265007_1 | AA376108 AA375646 AA376551 AW963816 |
| 301178 | 431366_1 | AA828385 AA769592 AA742234 AA742235 |
| 324042 | 266490_1 | BE387327 AA377589 AI903826 |
| 324048 | 267284_1 | AA378739 AW964174 AA570564 AI076833 AW265063 AW006805 AA480656 AW004789 |
| 324064 | 421551_1 | AL046841 AA721199 AL046079 AW137650 |
| 323409 | 218874_1 | AL135534 AA366767 AA313599 AA333787 AW966106 AW844387 |
| 323426 | 188613_1 | AA485236 AA830637 AA251401 AA749343 |
| 324094 | 270098_1 | BE395109 AW663898 AW237041 AI492154 BE046906 AI651285 AI983290 AW002590 AI201040 F32424 AA992272 AW271836 |
| 316231 | 427772_1 | AA732301 AA764985 AA732300 |
| 323479 | 194627_1 | AA278246 AW292815 AA278703 |
| 322840 | 109637_1 | AA083710 AA122195 AA081984 AA134650 AA080983 AA080954 AA062747 AA083876 AA079025 AA113855 AA074687 |
| | | AA078914 AA078873 AA074126 |
| 322877 | 116942_1 | AA079727 AA079728 AA824267 |
| 308447 | 10607_−2 | AI659985 |
| 324148 | 273755_1 | AA393624 AI190160 AI342032 AI015092 AA992047 AA860917 AA400867 |
| 300694 | 110146_1 | AA063406 AA063407 |
| 322907 | 5481_1 | BE252579 BE255945 BE314749 BE260141 BE260121 AA084941 BE206830 BE256911 BE256464 BE277162 AA448932 |
| | | BE206992 AA158178 BE265680 BE164838 BE164881 AA411651 AA036706 AA411652 AA480595 R53714 AK000381 AA191114 |
| | | BE314072 |
| 300707 | 114644_1 | AA079132 AA080921 AA076662 |
| 317010 | 485212_1 | AA863389 AA863395 AI969882 AI695443 |
| 324252 | 287622_1 | AW629965 AW975427 AA421989 AI051821 AW748356 AW129223 AA715470 |
| 300763 | 163257_1 | BE000150 AA190753 AA760634 AI685862 |
| 300781 | 23981_2 | AA731209 AA236961 AW971508 |
| 323676 | 220254_1 | AI702835 AI758919 AI685405 AI952108 AI299207 AI400767 AW105389 AI952710 AA845312 AI784118 AI537315 |
| 316473 | 439976_1 | AA811452 AA829961 AA815116 AA764935 AW976998 |
| 323699 | 222860_1 | AW178750 AW178736 BE141842 AW178683 AW178714 AW178684 AW178679 AW178716 AW178685 AW178718 AW178717 |
| | | AW178681 BE141834 BE141835 AW178711 AW178735 AW178712 AW178734 AW178715 AW178680 AW178713 AW178677 |
| | | AW178719 AW178748 AW178710 BE141843 BE141844 AW751119 AW178682 AW178737 AW178749 AA318993 BE141841 |
| 308659 | 3723_5 | AI174911 AF116710 BE250634 BE513651 BE513483 AW973114 AW973111 BE407498 BE387872 BE255662 AW575394 |
| | | AW157150 BE383339 BE045072 BE382560 AA535472 BE389253 BE274463 BE076283 AW157403 W20446 AW663763 N83897 |
| | | AI816181 AI879111 AA603287 AA809812 T03060 AA531063 AI815578 AA534177 F26418 AA527007 AW157453 AA557931 |
| | | AA742784 AW163466 AA513251 AI816220 AI816077 AI929498 BE299421 AW163056 AA502024 AW160897 AA651678 |
| | | AA534185 AW663233 H46043 AI815950 AA635243 AW161304 AA532646 BE619905 AW163000 AI928982 AI929122 AA569189 |
| | | H94752 AW163120 BE619294 N84679 AA639280 AW161846 AA524657 N76560 H60985 AA318407 R98156 AA316834 |
| | | AA534172 AA353056 AA375240 D16959 AW328044 AA603503 AA531077 AA330098 AA244141 AA377880 AW161054 |
| | | AA070576 AA326867 AA370759 AA378306 AA055146 AA311016 AA356827 AW827213 AA318418 N84051 AA322649 T72970 |
| | | AW630001 AA376419 AA343976 AA311126 F20273 AA308824 N84783 AA373006 AA374318 AA356480 AA300347 AA314040 |
| | | AW328346 AA092658 AA181308 C04940 AA329907 AA587591 AA300042 AA301309 AW674942 W21264 W47074 AA310460 |
| | | W31811 AW630251 AA332208 AA299335 AA360053 AA360318 AA328721 AA378343 AA299584 AA345747 T49611 AA376010 |
| | | AA186475 AW957009 AA305013 AI064911 AA330372 AV655922 AA314027 AA354481 AA313645 AA314699 AA314084 |
| | | AA358279 AA311170 AW327387 AW327464 D58829 AW973106 AA375740 AA308783 AA316940 AA315588 AA380257 |
| | | BE397919 AA181856 AI557351 N83566 AA315879 AA369434 AA357016 AA314419 AA130919 AA308111 AA314139 AA354095 |
| | | AA095733 AA247440 AA316663 AA307418 AA131136 AW157088 AA375200 AA316192 AA315616 AA360129 F26579 |
| | | AA363700 BE539203 AA379637 AA147978 AI340513 AA307861 AA467839 AA384626 AW328308 AW248834 AA379269 |
| | | BE621558 AA355664 AW404712 BE613747 AA507275 BE206816 AW270699 C13959 AW966785 AA310386 BE394115 |
| | | AA131219 BE385515 BE262339 AA095023 AA315277 AA364046 AA535741 AA188339 AA316095 BE312188 AA176667 T53669 |
| | | BE258138 BE258239 AA303587 BE272248 BE545416 BE250615 AA371845 AW839835 AA314570 AA330923 AA311279 |
| | | BE261310 AA320246 H95461 AA587557 AA308437 BE540153 BE390839 F25801 AA480575 N89472 AA302013 F29054 |
| | | AA375385 F19527 AI816119 AA227104 BE536936 BE535875 BE269659 BE536267 AA339654 AA771729 BE396514 BE263080 |
| | | BE312046 BE298027 AA771710 M13934 BE618296 AA807646 AI951409 BE613262 AA857346 BE294539 BE296371 F31870 |
| | | BE254168 BE621136 BE295564 BE612812 N88740 AA352787 AW475090 BE270398 BE618807 AI566936 NM_005617 |
| | | AW008274 BE392312 AI928878 AW162655 AI624147 AA854344 AW952982 AW085710 F01238 AA344919 AA641196 |
| | | AA720860 AA572870 AW161654 AI001113 AI862725 AI811592 AA320775 AI420790 AI207364 AI951381 BE270095 AI027794 |
| | | BE513425 BE252494 AA865409 BE294421 AI719767 AA897029 AI564328 AW628239 AI809839 AW082072 BE255143 |
| | | AI189653 AW515889 AA578771 AW162621 N86168 AA935172 BE384561 AW157031 BE295195 AI659958 AW263875 |
| | | AA533391 BE513648 AI445700 AI034465 H65716 AW272557 AW302286 BE271816 AW196996 AI953988 AW157372 |
| | | AA580376 AI191251 AA572856 AI027798 AA884282 BE268198 R92688 AW731956 AI261239 BE260070 BE253635 AA603422 |
| | | BE258354 AI810163 AA130398 AA861165 BE257787 BE408032 H96956 AA593399 BE275262 AA180066 BE395867 AI439795 |
| | | AA845816 AW572567 AA999943 AA301818 BE615972 T67509 AA152224 BE393104 AA857300 AA828978 AA812299 |
| | | AA834249 AW157807 BE253390 AA883769 BE382656 AA484099 AA759089 AA501594 AA742887 BE391312 AA304434 |
| | | AA091576 AW939788 AA989550 AA301655 AA374299 AI000013 AA379938 AA985668 AA293111 AA838481 AA302129 |
| | | BE255097 R48458 AA659479 AA311090 AW262218 AA864655 R70415 AA782420 F29372 BE301374 AA345410 AA152252 AA664047 |
| | | AA626811 AI092022 AA300592 AI204107 AA936560 AA349088 AI365000 AA311155 AA854688 AA176832 |
| | | AW338622 AW246720 AI096811 BE390455 AA181218 BE274363 AA320085 AA854819 AA379935 AA961683 AA706429 |
| | | AA301855 AA903668 AI167880 AA912490 AA736633 AA978322 AA306985 AA492120 AW613649 AI026088 AI500692 |
| | | AA642146 AA834125 AA970750 AA954809 AI937927 AA605094 AI000445 AW592871 AA642613 AI708961 AA864208 |
| | | AA935984 N78708 AW263613 AA379400 F30530 BE382934 AI347719 H44065 AA876331 AA642115 AW439014 AW613635 |
| | | AW592031 H02916 AI720155 AA661949 N24052 T72191 AI343917 AW000828 F20434 AI040902 AI189222 AW615066 |
| | | AI364998 AI833275 F20713 AA884055 AA603404 F32779 AI927611 AA582556 U91916 H44037 AA641501 AI718241 H44109 |
| | | AA971206 AA782740 AA311211 AA947800 N23272 AI590880 AI742000 AW651617 AI040892 AI051117 AA554857 AA782075 |
| | | R19307 AW515187 AA622350 AI284864 AI002194 AI339662 AA535783 AA480434 AI951550 W46973 AI936248 AA492016 |
| | | AA972511 AA071036 AW170504 AW629056 AA658282 AA843949 AA846986 AI335690 AI985054 AI335679 AI660582 F26201 |
| | | AA810379 AA187195 AI274712 AI300958 H99863 AA563821 AI973025 AI186080 AA533122 AI205703 AA166781 AA857765 |
| | | F37969 AA578216 F28780 AI972670 AI709163 AI749751 AI087008 AA186713 AA506638 AI298400 H62141 AA524656 |
| | | AA532581 AA136078 R48459 AI002336 AA617627 AA130399 AI870668 AI991186 AA293498 AW088368 AI929232 AA143219 |
| | | AA629414 AI859102 H73727 AA186706 AI581697 AA747261 AI718130 W73976 F26566 AI816041 AA633944 AA878094 |
| | | H61558 AI857355 H45978 F25943 AI879870 F27056 AA244085 AI927367 W69664 W69843 AI298014 N91111 AA229474 |
| | | AW969539 N95295 AW023874 AW572321 AA481820 AA580774 AI870680 AI568052 AI831105 AI750091 AI141652 AI833250 |
| | | AW001251 AA180067 AI554189 AI719251 AA807645 AI298013 T52779 AW264018 F21944 AI253714 AA342880 AA985284 |
| | | T49612 AW263695 AA508894 AA181908 AA533851 N84414 AI277062 T78929 AA534250 AA569444 R11589 AA022468 T60272 |
| | | AA582353 AA335709 AI929540 AW613761 BE327851 AA533776 W72345 AI601210 AA366049 AA719887 F22515 AI523442 |
| | | T07133 AA230206 AA827438 AA728986 AI718076 AA504069 AA469341 BE546614 AI434088 AW170241 T52780 AA629409 |
| | | BE543516 AA484074 W04709 AI582317 BE045496 AA737847 AA535491 AA628352 AA558076 AA864201 AW610533 AI707857 |
| | | R94198 AA522868 AA226911 AA857018 AA548787 AA627142 H65717 AW166287 AA564007 BE547258 AA330345 C15020 |
| | | AA168754 AW244086 AA844918 F32317 AI424155 AA228718 N89046 N91894 AA894498 AA995502 AA533354 BE222986 |
| | | F27737 AI887199 AA506272 AA558503 AA578345 F22227 AA352404 AA805843 N88511 AI628216 AW341732 AA300401 |
| | | AA648148 F19238 AI200285 BE041727 F29059 T92628 AW589349 F30549 AA653059 H61217 AA501652 AI919597 AA903957 |
| | | F31025 N34998 R96850 AA938689 AI919025 AI310209 AA975769 AW338225 AA342924 F25408 AA130863 AW087264 T72994 |
| | | AW873946 AW327429 AI186381 AW327428 AA974301 AW768990 AI690826 AA652111 AA467888 AA187218 AI183905 |
| | | AI424323 AA548057 AW051027 AW080306 AI803806 AA642861 F31183 AW406529 AI619660 AA918081 AA491673 F21729 |
| | | AI874177 AA715051 F33182 AA652773 AA659091 AW339904 AA662483 AA857275 AW105417 AA548056 AA601164 |
| | | AA622675 AA494257 AA036629 T60772 AA653018 AW673531 AI613161 AA668312 AI817429 AW195254 R93615 R69436 |
| | | F02292 AA995267 AA247239 AA320193 AA782878 AA994819 AA054936 AW151254 F17418 AA908823 AI890234 AA534130 |
| | | AI468569 AW149483 N22520 AA343231 AW001178 AA602740 AI352348 AA932611 AW273549 AI766324 AW118402 |
| | | AA888486 AA503847 AW300756 AA585120 AA496674 AA629998 AI619509 AI799389 T25968 AA535332 AA535126 AA467830 |
| | | AI915716 AA320648 AA585125 AA658579 AW451040 AI735214 F16590 AA284984 AA643858 AA283217 BE178572 AI921154 |
| | | AA344918 AA557543 AA308479 AA872892 AA481963 AA658578 AW004717 AA290824 H61178 AW474724 AA321490 |
| | | AA936492 |
| 308681 | 352627_1 | AI761307 BE161415 AW005281 AA558937 BE161404 |
| 324316 | 298498_1 | AI291330 AA452220 AA443354 |
| 324323 | 273867_1 | AA393739 AL138137 AA393796 |
| 324368 | 33663_1 | AW861185 AW991306 AI623237 AI018451 AA159072 BE294536 N40680 AA248594 AA332177 AA353889 AW136671 |
| | | AA063492 AW082601 AI734889 AI479298 AI690289 AA514475 AI827201 AA954932 AI375429 AW305231 AI078116 AI807586 |
| | | AI224855 AW025809 AA718988 AI093059 AI912915 AA456139 AI341411 N63437 N95744 AI830516 AA846245 N25902 |
| | | AW469026 AI042513 AI241692 AA988159 AW662812 C03567 AA594584 AW082516 BE068964 AW299374 AI636034 |
| | | BE172420 AB029022 NM_014914 AW896987 AA765269 BE077785 AA459768 AA436896 AL040331 AA285281 BE244301 |
| | | T54938 AA093121 F21778 AA150155 AW968251 AA258877 AW663766 AW470130 AI857358 AI469756 AI299480 AI672560 |
| | | AW089174 AW518770 AA081121 AW014387 AI869379 AA985193 AA152265 W94876 |
| 324376 | 1154206_1 | AW499705 AW502537 AW503016 |
| 323735 | 6884_44 | BE615406 AA323714 BE515303 AA464826 AA453149 AA442802 BE387695 BE394179 BE392472 BE394406 BE393601 |
| | | BE394088 BE388690 BE395181 |
| 323755 | 229275_1 | AW300094 AA805026 AA700077 AI702882 AA327146 |
| 323770 | 230698_1 | AA722425 AA329212 AI572177 |
| 302138 | 228984_1 | N83965 AA326737 H14153 |
| 325007 | 428607_1 | AA931484 AA758140 AI033284 AA931461 AA757895 AA877176 AA736429 R11591 AI017256 |
| 302162 | 24588_1 | AF119046 NM_014481 AJ011311 AB021260 BE091822 BE091819 BE280661 BE548576 BE531337 AW605371 BE547006 |
| | | BE076387 BE275532 AW386829 AW189348 AA554484 AW028935 AW080283 AW444648 AI538979 AI674393 N59092 C01178 |
| | | AI612817 AI347385 AI190577 AI918533 BE265083 |
| 302183 | 25243_1 | NM_002248 U69883 F11363 M62043 AA350009 AI937237 AI363154 AA351095 AA325431 D60120 D61063 D60321 C15510 |
| | | AW292489 BE504989 AW594619 AI825516 F09023 |
| 324432 | 312487_1 | AA464510 AA631257 AI740516 AI739132 AW972467 AI741376 AW068935 AI467852 AI752240 AI123717 AI754551 AW205510 |
| | | AW044211 AW028889 AW198033 AI538632 AA513096 |
| 323808 | 247059_1 | AW250114 Z43124 AA431421 AI879054 AA351616 AA351035 AL048999 |
| 300982 | 361671_1 | AA837754 AA581115 |
| 323853 | 238221_1 | AA393460 AA338940 AW966277 AA419006 |
| 316664 | 455916_1 | AI042101 AA805541 AA812130 AW977868 |
| 323899 | 48373_1 | AL042966 AW403294 BE294048 BE274337 BE261318 BE544865 |
| 309470 | 1028089_−1 | AW118833 |
| 308859 | 995_8 | AI830787 BE563334 |
| 302263 | 227972_1 | AA325517 H19549 R53308 |
| 302270 | 1734192_1 | R56151 W91936 |
| 302292 | 27735_1 | AF067797 AB013456 NM_001169 AI791955 AW843925 AI732659 AA577625 AW083143 AW138645 |
| 301644 | 867855_1 | AW239364 AI792390 |
| 301654 | 103655_1 | H81795 Z42291 R20973 AA046920 |
| 301660 | 171572_1 | F13112 Z42752 T77015 AA211163 |
| 324538 | 1156268_1 | AW502082 AW502979 AW502807 AW501876 |
| 301685 | 326972_1 | W67730 Z44630 AA490699 W67596 W76661 R21207 |
| 324560 | 1156518_1 | AW502208 AW502366 AW502148 |
| 324568 | 1135567_1 | AW502311 AW502370 AW403858 |
| 324575 | 65704_1 | AW502257 AI014241 AA100360 BE298534 |
| 308994 | 79433_1 | BE261877 AI880051 BE242392 |
| 302334 | 29269_1 | AF120491 AF187963 NM_004980 AF205857 AF048713 AL049557 |
| 301703 | 916575_1 | AW301478 AW301560 AI889207 |
| 301714 | 1534796_1 | F06529 R19994 |
| 302399 | 1570217_1 | N79624 H18620 |
| 301744 | 50221_6 | BE277526 AW854299 AA356083 BE257389 F12667 T74316 BE251675 |
| 301752 | 259807_1 | F13486 T75247 AA368304 |
| 301767 | 8416_1 | AK001579 H15317 AW361892 R38240 R02257 R41005 T84341 AW963098 AW516794 AW664706 AI377637 BE463865 H15706 |
| | | AI452461 AW089869 AI986480 AA636063 AI123920 AI911424 AI123930 AI963282 AW170740 AA564654 AW136832 |
| | | AW468248 AI393655 AI656932 AI358006 AI968742 R38241 AA928629 R77428 AA705932 AA458491 AI755108 AA913331 |
| | | AI351287 AW044222 AA301591 R81470 AA344045 |
| 301775 | 24697_1 | AW250572 AW247670 BE067043 BE067049 AL080159 T48255 AI628030 AI401612 AW246476 AA365210 T48256 T81697 |
| | | H62983 AA909497 AI864565 AI801862 AI247137 Z25119 |
| 316893 | 473541_1 | AW979189 AA837332 AA856946 AA876935 |
| 316897 | 474090_1 | AA838114 AW629478 AA883713 AI620552 |
| 303021 | 110136_1 | BE277604 BE276408 BE265007 BE410545 BE407265 BE272589 BE263600 BE407970 BE303032 W39612 BE244678 |
| | | AA321272 BE244631 AW405664 AW673342 BE244379 BE280236 |
| 303041 | 31134_1 | AK000072 AW840683 AW843764 AW844444 AW844515 AW603469 AW862395 AI860838 AW511708 AF127035 NM_012128 |
| | | AK000138 |
| 303042 | 5058_1 | AW505345 AF129532 AF126028 AA852108 BE169359 R83701 Z43904 BE613543 AA283163 AA905463 AW067849 R13544 |
| | | R12337 R14020 H98970 AI474918 N56139 AL135669 AW067702 AW372065 AW631389 AA083416 AA287511 AA602923 |
| | | AA488914 AI167215 AW946829 R82855 AI948792 AA371333 AW953883 AW956152 C02539 AA298280 AI932587 AA022742 |
| | | AI983021 AA195252 N58991 R78733 AW083996 H39614 AI365249 AW615389 AI927744 AI089971 N52205 AA083417 |
| | | BE326666 BE349514 AI743785 AI640148 AI378211 AW181881 AI949484 W31374 AW628233 AA418406 AW068010 AI708085 |
| | | AI092696 AI089823 AI277828 AA022660 AI440527 AW054937 AW474104 AI017436 AI159819 AI356716 AW473140 AW316518 |
| | | N34522 AI675092 AI866697 AA864593 AW511185 AA488844 AA904975 N49111 Z39951 R37265 AI141362 T25856 R20664 |
| | | F03163 AI767927 AA805942 D79905 AI914645 AW190553 AI934213 AI458796 AA195385 R82854 W31965 |
| 302445 | 16351_2 | W37653 AW881001 AW840685 AW840696 N79647 AV648691 H25494 AW885394 AW001940 AI362793 N63970 H25448 |
| | | AI963184 AA324156 W37514 |
| 302459 | 31751_1 | AF169255 NM_006028 AF080582 |
| 302488 | 32135_1 | AC005551 AF161441 AA431001 AA336054 AW965560 AW376266 AA758808 AW024675 AA946948 AA306783 AA910368 |
| | | AA335971 |
| 301855 | 33235_2 | W25940 AF053356 AF053356 |
| 301859 | 1799608_1 | T61587 T61035 |
| 324783 | 389615_1 | AA640770 AI683112 AA913009 |
| 316979 | 483302_1 | AA861087 AI200951 AI026779 |
| 303100 | 44550_1 | T09353 U72943 U64597 R52598 R61404 R20022 R18898 U67037 |
| 303106 | 85273_1 | AA012877 AA021074 |
| 303144 | 40757_1 | AF202889 AF202890 |
| 302514 | 2628_10 | X58801 X58802 X58803 X58804 AI312844 AA283290 X51791 X86140 X51795 X86110 X86106 X86105 X86104 X86141 |
| | | AJ296352 AF020642 |
| 303179 | 112488_1 | AA071215 AA071444 |
| 302535 | 1589284_1 | H48676 W04984 |
| 302569 | 17513_2 | AC004472 BE312721 BE273942 F11928 T65358 BE612432 BE261576 BE179884 |
| 318230 | 193526_1 | AW407564 AA262049 W19405 AA504733 T12641 AW973724 AA558125 AW993087 T12640 T11447 AA521285 AI820042 |
| | | AA457028 AI674737 AI688648 N92749 AI439620 AI218005 AA731321 AA828303 T12590 AI685371 AA504636 AA825573 |
| | | AW172614 AA291292 AA649919 AW576505 AA262152 H29888 AA830757 AA910433 AA808481 AI971807 AI767874 AI216422 |
| | | AA831483 AI566351 H41305 AA879438 AA807123 |
| 324829 | 7288_14 | AI537407 AW131277 AA714311 AW816039 |
| 311004 | 344894_1 | AW191647 AW973241 AA632846 AA533681 T51580 |
| 309825 | 331299_2 | AW293701 BE348286 |
| 303215 | 130271_1 | BE281058 BE276139 AW250314 BE336754 AA112498 BE265334 AW732954 |
| 303265 | 155956_1 | AA171403 AW160951 |
| 303294 | 72494_1 | AA205300 BE612585 AI393918 |
| 302646 | 2628_31 | X58808 AF071471 |
| 324945 | 47710_1 | AL360202 AA088768 R61018 T36114 AW966404 M85891 AW296810 AA421905 H29985 AI810129 AI798786 AI338041 H29886 |
| | | AA088702 R60901 AI648560 AA411400 |
| 309912 | 14912_1 | H19255 AA324634 BE398078 AW749970 AA309733 AA769449 BE501906 AI686211 AI280426 AI401680 AW235634 AI954773 |
| | | AI762656 AW082924 AI223790 AF160973 AI127758 AW009952 AI440473 AW136694 AW008937 AI492112 AW294956 H12044 |
| | | AA609256 AI400622 AA778657 AI342773 BE501767 BE048879 AI187333 AW025304 AI191550 AA383780 BE350769 |
| | | AW024606 AI244835 AA228447 AA983651 AA027332 AI167499 AA913695 AI479796 AA960843 AW272340 C02052 AI433299 |
| | | AB032994 AL136549 AI126781 AI339656 AA324178 AW274942 AW404411 AA351287 AW503975 AA978021 H19254 |
| | | AA826207 AA365760 AA887252 W35146 AW299327 AW593902 AA932287 AI940307 AW955721 M62008 AA034345 BE048271 |
| | | AA365723 AA682987 AA324179 T08342 BE396592 BE269097 BE242683 AA351288 AA317743 AA323517 AW950991 |
| | | BE169813 AW500399 AL119581 AW401530 AW501601 AW501500 AA663641 AW892664 AW501520 AW501448 AW501591 |
| | | AW500843 AW499580 BE396829 AA143673 AI834230 AW500792 AI909739 AW845214 BE140556 AW845213 AW500505 |
| | | AW500506 AW845216 AW574989 AI834238 AI929491 AW576513 BE245479 BE241953 AW505039 AI263367 H12043 |
| | | AA033638 AW339671 AW963748 |
| 309963 | 22431_1 | AW449073 NM_016507 AF227198 BE062563 AW893244 AI695667 AW370607 AW370608 AW505230 AL121377 T19010 |
| | | AA397553 AI138391 T86408 R78328 AA829647 AI797062 AW502286 AI703352 AI298289 AA730053 AA047200 AW083099 |
| | | AW370617 AI128124 AI264202 AI350744 |
| 319000 | 1534458_1 | Z44318 F06149 R20136 R14907 |
| 319003 | 174731_1 | R18085 AA219028 R17712 Z44345 |
| 319027 | 6267_1 | AK002182 AW842140 BE391303 AW807017 AW604195 AW604194 AK000527 AA716612 AW291848 C17004 D78698 |
| | | AA633316 AW085548 AW954620 AA351081 AW377003 AA594606 AA290880 R35913 Z44526 T74413 H39831 H03611 H70235 |
| | | AA410732 AA404618 AA026902 N87562 R49433 AI346545 H03501 AW189821 AA235590 AI188349 AW262971 AA283775 |
| | | AI431671 AA026903 AI246417 AI636152 AI370910 T74039 AI128930 BE208355 Z40426 AA291429 R01208 AI824714 |
| | | AW075793 AA576559 |
| 302722 | 44456_1 | U53530 L23958 AA775976 BE003131 AI903793 |
| 303388 | 969232_1 | AL039604 AL039497 |
| 319055 | 167464_1 | Z44855 AA196906 AA196723 BE278458 AA412305 |
| 302776 | 23857_1 | AJ133798 NM_014427 AJ133799 R19516 AA071126 AI278055 AA071125 AI915130 AI582322 AW070237 Z39757 |
| 319091 | 1237615_1 | BE392792 H55196 Z45264 |
| 319098 | 922966_1 | AI908374 Z45366 R11479 H79927 |
| 317894 | 226420_1 | AA323682 Z44357 F11306 R49705 R60252 T09440 T09172 H09876 F06094 R60848 H06625 T07793 |
| 303405 | 215022_1 | AA308601 AW411014 |
| 303407 | 6848_4 | AB040958 AA309616 AA322942 AA331675 BE257729 AW806959 AW860144 AW806968 AW512751 AI239617 R68424 R72046 |
| | | H85465 N28472 AI971920 AI459052 AI082821 H49714 AI218903 AA244036 |
| 318504 | 1703724_1 | T26453 Z44226 R20425 |
| 318563 | 7543_1 | AB037724 BE260227 R21318 BE538345 AW250501 AW403023 R46363 Z43008 R14654 AW953148 AW953149 T33110 |
| | | R20231 H30838 F05454 Z45790 AW965595 T08819 H06568 H29913 R16617 AW006449 AA588448 AI909629 R10832 |
| | | AW245067 AI479004 H29825 AI097108 AI829928 AI636607 AI079390 AA353858 AI374890 BE247041 R43324 R10833 |
| | | AA603730 AI298051 AI580953 AA400088 R16559 AI369715 AA401378 AW976688 BE245314 AW249837 AI869249 AA349796 |
| | | AA783040 AW474996 AI028378 R45633 AI864452 AA470470 AA629126 AI262201 H06513 |
| 304161 | 34056_−2 | H71886 |
| 311395 | 252948_1 | R23313 R23323 Z25059 AA359123 AW965886 BE167187 AI808503 |
| 303521 | 244029_2 | AA746272 AA504079 AA347316 BE084755 |
| 303535 | 972533_1 | AL043430 N79419 |
| 319207 | 1071625_1 | R87679 H16883 F07785 AW245503 |
| 302919 | 34022_1 | AL137382 AA305545 |
| 303592 | 29065_6 | BE145971 BE146046 BE146316 BE146148 AA421129 AA382152 AW836960 AW836945 AW836851 |
| 303596 | 270128_1 | AW303377 AW270092 AA382636 |
| 319264 | 247031_1 | T65096 F12013 AA351004 F11787 T66093 |
| 302972 | 47307_1 | AF086321 W73400 W73375 |
| 304211 | 21366_−11 | N62228 |
| 304241 | 84545_1 | AA010976 AW611600 |
| 303642 | 284260_1 | AW299459 AA417112 |
| 303650 | 290929_1 | AA430709 AA427966 |
| 319359 | 1541128_1 | F13458 R17813 T74431 |
| 319373 | 226757_2 | R00371 R09720 R09711 AW873586 AI703426 AW016769 R36969 R00259 AI216256 R09712 |
| 318728 | 323486_1 | Z30201 AA486132 T72025 |
| 312129 | 338029_1 | T87431 AW300867 T87330 AA515973 AI242970 |
| 312162 | 423491_1 | AI660245 AI950027 AA971142 AI302981 AW771990 AA723809 AW195235 |
| 303745 | 54759_3 | AI142379 AA905379 |
| 319403 | 1688716_1 | T86043 R01431 T75579 T75580 T83765 T98413 |
| 303778 | 174437_1 | AW505368 AA218610 F11852 T65345 AA397806 |
| 303782 | 2213_1 | AA325129 AV655735 AW408703 AW505411 T64737 |
| 319468 | 1691029_1 | R06504 T99600 T95769 |
| 318813 | 1540877_1 | F13195 Z42617 T75318 |
| 320042 | 551394_1 | AI017510 T91374 |
| 320083 | 1764305_1 | T87761 R95026 W87407 |
| 312217 | 382018_1 | AA627706 F28433 D63189 |
| 312251 | 193841_1 | AA323400 AW968650 AA262398 H03952 AA824430 |
| 312281 | 1563872_1 | H46445 H22490 H19702 H46328 |
| 305064 | 11422_−7 | AA636012 |
| 303835 | 1305867_1 | T05645 T32180 AW961890 T08680 |
| 303849 | 43707_1 | AA249439 BE256642 AW163324 AF012357 AL359617 H95481 AI217156 AI332310 AI016751 H94962 AW105657 AW082779 |
| | | AA815334 AA954302 AA628321 Z42118 AA737794 F08727 F06339 F07626 AI829971 T89928 AW970824 AW157525 AI979018 |
| | | AI537885 AI636077 AW439894 AI687692 AI476254 AI870810 AI860351 AI248264 Z40675 F05012 F02632 AA564443 |
| | | AW298565 BE091953 BE091884 F01656 BE091886 BE091841 BE091851 BE241581 AA403051 |
| 320115 | 582314_−1 | T93574 |
| 320121 | 452027_1 | AA780365 AA909233 AI275542 |
| 318946 | 92484_1 | AI122843 Z43800 R19718 R59259 N74752 W20097 N46928 AA215691 AW672684 AW672673 AL044945 H93163 H92772 |
| | | AW451096 AW675432 AW675425 AA844417 AW674797 AA855104 AA291320 AA770259 AI749837 AA034362 AW370463 |
| | | AI825998 AA027067 AA744373 AA732336 AW674014 AA291321 AA029285 AA766450 R59201 AA588329 AI168449 AA215692 |
| | | AI079651 Z39868 R05650 F03066 AA357760 AW955291 AI992075 AW020148 N90519 |
| 312339 | 151127_1 | AA524394 AW015969 AA158731 AI831401 AI955800 AW272596 AW272595 AI281799 R34231 R35558 AW378527 |
| 304576 | 25108_1 | AA329384 S49006 AW405735 AW404287 AW405848 AW405979 BE538908 H70947 AI814190 AA381896 AW582731 |
| | | AW975580 T94914 AW474500 AW869616 AW974125 AW946246 H44560 AW802184 AI538108 AW609912 AW865715 |
| | | BE007659 AA443571 AW376161 AW797783 AA713566 AW577973 AA745472 AW270172 AA910107 AA713717 AW364122 |
| | | AA533898 AI523353 AA715899 H44507 AW947263 AW390753 T89943 R66359 H44320 T64100 AA715879 R54661 AW579645 |
| | | H68597 AI499528 AI699010 AW605041 AW605043 AA236700 AI290767 H27472 AW079688 T69967 T87728 AW869468 |
| | | AW873389 AI250670 AA617786 AI566627 AW627605 AW302334 AA617836 AW793186 H16015 R83107 T85409 AA643479 |
| | | AA582449 AW794654 AI819995 AW865810 AW380149 AW793863 AI281629 T64021 AA501599 R49882 AA565066 R69406 |
| | | N23918 T59642 R54662 AW973199 R53006 AA401087 AA496563 AW934874 BE171294 AW793133 AI581595 AW605835 |
| | | AW605040 AW605055 AW605033 AW605058 AA291844 AW382472 AW605005 AA421881 AA513106 AA744190 AW386065 |
| | | AW613785 AW605829 AA580905 AW605027 AW364134 AA464027 BE012096 BE008565 AA809035 AA809154 AA911395 |
| | | AA713636 AA745324 AA826230 AW368498 AV646747 T89967 H61164 AI540661 AA721571 C02031 AA523762 AA523746 |
| | | AI287256 AW841164 AA962139 AW797014 AA515246 AW364140 H45842 T94267 T72111 R49771 T70151 H42664 R83656 |
| | | R83590 AW380297 AW380263 X95750 T57717 AW391436 AA293355 AW380185 X95749 X95748 X95747 AW796510 |
| | | AA318628 AI001043 AA744404 AA745567 AA650437 AA715526 AW392762 AA516228 |
| 303958 | 10830_7 | AL042931 T70163 |
| 319668 | 17848_1 | NM_002731 M34181 AA091596 AA090564 AW498491 AA383248 AA018979 AA352938 AL133918 T28731 AA984398 AL134676 |
| | | AW898293 M77877 AI124701 BE567802 AI288466 AA719614 BE622570 |
| 320236 | 321288_1 | AA528811 AA482942 |
| 320258 | 217516_3 | BE396475 AW814056 AA311866 H05207 R94015 AA504370 AW962851 T70351 |
| 319793 | 1698018_1 | R56360 R20336 R67291 R14290 |
| 320360 | 259694_1 | H12405 H12404 AA368159 |
| 319886 | 22717_1 | AK000906 AA603325 AI123244 AI742610 AI742621 AW451297 AI963292 N94395 AI122654 AI869312 AA693938 AI913241 |
| | | R69552 AI925715 AI243319 AI085752 AI094469 AI922008 AI521658 AI288451 AA627433 BE327863 AI417213 AA937953 |
| | | AW301143 AI827213 AW444808 AI056464 AA736786 AA968590 AI425075 AI420510 AI445367 AI866676 H25733 AI356146 |
| | | AI803734 AI194056 AI872656 AI222629 Z38818 H25937 AI547263 AI656165 AW590474 T73549 BE392068 H14735 H27088 |
| | | H45935 AA359427 AW167511 |
| 306023 | 38159_2 | BE463655 AA897764 |
| 313257 | 452953_1 | N92638 AA781521 AA846225 |
| 305406 | 34836_−76 | AA723860 |
| 304831 | 13890_7 | AA314337 AI805587 AA402097 AA293631 AA658356 BE566333 AI557276 AV651311 NM_001011 Z25749 AI525506 R97869 |
| | | BE546683 AA155861 AA532947 AA329269 H63470 AA187471 AV660956 AV650019 AA380909 AW327895 BE313243 N93984 |
| | | AA354585 W19057 AA428966 T55686 AW958123 AA315872 AA081305 AA083776 AW961442 AA352331 H00860 AA206205 |
| | | W21515 AA149180 H13044 AA372080 AV658875 AV658874 AA337727 BE276865 AA247683 AA209449 AA393619 AA147378 |
| | | AW404839 W19374 AW247081 AA311228 BE615971 AA308572 W01826 AA310230 AA412606 AA420575 AA362646 H70797 |
| | | AA525988 AA311440 BE564044 AW967302 AA302565 AA313021 AA312653 D52864 AA312582 AV654672 AA308096 |
| | | AA308509 AA308491 AA305867 AA306813 W75940 AV653876 AA365315 AW408185 AA308217 AA122395 AA314083 |
| | | AA315981 AA311782 AA310647 W46668 N31114 AA180871 AA187276 N72805 AA312418 AA304985 AA167580 AA307881 |
| | | AA310961 AW674912 AA310126 H82669 AA132497 AA809088 AA155963 AA640361 BE618580 AA229152 AW404970 |
| | | AA306621 AA308276 N98583 AV660766 AA654587 AA206190 AA312305 AA580740 AI568153 AI718253 AI860477 AI872388 |
| | | AA873460 AA405147 AA315435 AI129479 AI888709 AW583033 AW089596 AW264185 AI653503 AA858344 BE619226 |
| | | AA187721 AA306691 AW439857 BE616042 AI889905 AI862869 AA309960 AA307823 AI148769 BE619772 AA582168 AI963414 |
| | | AW168760 AW167856 BE619400 BE378974 BE619933 AW958117 W49703 AI951380 AA306263 AA583192 AA356605 |
| | | AA897095 AA149141 T53975 AI744255 AA845352 BE312890 AW961435 AW583101 W24684 BE619333 T96513 AI491903 |
| | | BE272296 AA315556 BE539380 BE619999 AW084687 BE465646 BE262138 AA999923 BE260616 BE276306 D55310 AI186578 |
| | | AI707735 AW404371 BE274614 AW512395 AA650425 AI439470 AA513739 AW404673 AW404664 M77233 AI970731 AI880754 |
| | | AI418821 AW273410 AA069936 AW058084 AA719351 AW404422 AW405314 AI523106 AA393107 BE408961 BE252375 |
| | | AA578681 BE513748 AI749494 N36979 N43025 D11774 AW391958 R34130 BE394953 AI865715 AI018059 N46292 AA908866 |
| | | AA907443 AW081633 AA622318 AW438511 BE262297 AA565643 AI018057 AI440067 AI719291 AA135054 BE222834 |
| | | AW262240 AA081120 AA442581 N86460 AA583569 AI061357 W72482 AA148278 AI186761 AA512934 AW615804 AA595332 |
| | | AI831069 AA778136 AA657641 AI433175 AA398698 AI735084 BE183158 AI566940 AW304785 AI401846 AA506149 AA102508 |
| | | AA736531 AI027939 AW000716 AA861339 AI027937 AA575877 AA639090 AW129111 AI421179 AA506553 AA936780 |
| | | AA972869 AA563975 AA582448 AI802748 AI471162 AA586422 N86248 AA528142 AA551225 AI566307 AI206730 AA513485 |
| | | AA363629 AA436824 AW575828 AA180774 AI001103 AA932070 AA613667 AI337566 AA582127 AW327319 AA610833 |
| | | AI333738 AI708124 AI349480 AI240444 AI225152 AA570790 AI539813 W80674 AI333517 AA181299 AA315527 AI719857 |
| | | BE087137 AW474616 AI510739 AA614724 AW337476 AW951820 AA149181 AA149142 AA523097 AA428873 AI708760 |
| | | AI978984 AA946913 AA844685 AI440225 AA306527 BE349026 AI659593 AA845752 AW327366 AA311945 AA595594 AI027199 |
| | | AI499554 AI262336 AA398535 AI333388 AW575598 AI472745 AI476623 AI150520 AW897766 AI864033 AW584030 AA148411 |
| | | BE348977 AA420550 AW768575 T27844 AI349314 AI371037 AI675707 AW102842 AI203978 AI719821 AW768495 AW021575 |
| | | AI686981 R36541 AI346551 AW888705 R25390 AA209335 AI342426 AA056446 AA582981 AA890201 AW026850 AA857587 |
| | | N91141 AW768541 T55235 AA401942 AA080860 AI291273 AI312669 AA412271 AA890649 AA846264 BE019907 BE090895 |
| | | T56904 H70798 AI500573 AW327642 F17734 AA157192 AA152417 AW327670 AA985481 H71524 AW439687 AA968516 |
| | | AW327857 AA405330 AI342315 H01239 AA878156 AA148279 H71525 N52399 AI991878 AA878100 N93956 H56020 AA132515 |
| | | W49704 H63414 AI717990 AI814318 AA912383 AA922317 R97819 AI860255 AI335093 AI187843 AW270357 AA187646 |
| | | AI190237 AI991126 W07024 BE539453 AI819841 H75424 AA102446 AW026974 AI923973 AW001389 AA337759 N70772 |
| | | AW022155 AA336624 AI219252 N89590 AA664546 AW339690 AA651993 AA608735 H65064 AA857788 T81916 AA860215 |
| | | AI031685 AA133689 AA186954 R16825 N27213 AA857155 N69981 AA318664 AI735334 H82411 AI460293 AA205819 |
| | | AW803036 AI922965 N33363 AA580622 AA664756 AA651961 AI201624 AI066494 AA834187 AA187722 AA652066 W80675 |
| | | AW051553 AW263699 R34131 AA340971 AA364263 AI824574 AI356913 AI439263 AA205822 AI540881 AA640625 AI206794 |
| | | AI198883 AA167581 AA970474 AA299068 AA157088 N90120 AI371330 AA090914 AI889887 AI274617 AA730747 AI745000 |
| | | AW265227 AI358313 AA122396 AA501489 AA918963 AA953637 AI434343 AI707767 AA603889 W04799 T55604 AA614231 |
| | | AA071264 AA083777 AI880809 AI185259 AA747112 AA908459 AA134970 AI192649 BE046107 AI358698 AA310968 AW517574 |
| | | AA984892 W46578 AW105597 AA224970 AA747937 T96514 AI472689 AA283670 AI186789 F35482 AW873463 AW361039 |
| | | AA583072 AI872660 AA040277 AA224895 N85343 AA484723 AA937597 AI304419 AA228409 AA501488 AA664896 AA664966 |
| | | AA482963 AA736806 AA528814 AA927560 AA328094 N83225 AA582536 AA938377 AA916871 AA507716 AA101182 AI720885 |
| | | AA876858 AA483322 AA362647 N75592 N23917 AI193949 N74054 AI352629 D51754 AI570783 AA470923 AW975162 |
| | | AA328256 AA659768 AI919411 W40458 F00799 AA147346 AA728856 AA528675 AI676101 AA523785 T52866 AA533842 |
| | | AW516442 AA554875 AA886066 AW518472 AA971169 AA908345 AI561318 AA482746 AA482735 AA482882 AA188350 |
| | | AA629989 AA482917 AW263351 T52867 D52318 AI696386 AA689276 AI719128 AI719124 AA541702 X85626 AA640165 |
| | | AA228408 AI832575 AI143550 AI281727 N73099 AA733095 AI749387 X85625 AA640766 AA652170 AA652139 AW606146 |
| | | AA649563 T25406 AA327330 AA080984 X74803 AA746016 AW957997 AI005578 BE547070 AA886825 AA927742 AI927866 |
| | | AA528573 AA665260 AW270340 AI698507 AA501524 AA483113 AA506345 N91789 AI933429 AW904843 BE092709 BE093487 |
| | | BE093472 BE092712 F22641 N80062 AW455220 BE079292 |
| 319900 | 1138215_1 | AW408392 AW847299 AW847302 T86065 T85884 T77135 |
| 321121 | 1545647_1 | W23285 H42714 F25381 F37215 |
| 321132 | 117535_1 | AA081495 R87345 H43858 BE266428 BE263090 |
| 320503 | 25164_1 | NM_005897 AF156857 AA346876 BE545147 AI003306 N45644 AW889728 BE007236 |
| 320586 | 26170_1 | NM_003658 AF031924 AJ243512 AI792204 AI675861 AI915798 AI733496 AI823489 AI370544 AI696665 AA991537 |
| 305557 | 41560_1 | AA774834 AK001079 BE293936 AW372365 BE271898 F00113 AA424108 AI950409 W70049 N56481 BE046416 W70050 |
| | | AI619669 AL040446 AA996189 AA962427 W37166 AA179135 Z21574 N64837 BE562818 |
| 304978 | 28800_7 | BE259406 BE379716 AA147786 BE300171 BE267161 BE515287 AU076914 AW374229 AA069647 BE561555 D53431 D54395 |
| | | AA191250 D52650 BE208576 BE566731 BE387383 AA373416 R23516 T68691 T69334 AA355791 R02636 BE566591 |
| | | BE407444 AI982546 H49682 AA094446 BE515225 D54740 AA095798 AA196716 AA353034 AW955137 D53869 AA100150 |
| | | AA188091 AI524940 D54982 AA090373 D53479 AA247363 BE567604 BE391813 N41527 BE410575 AA380139 BE616028 |
| | | AW881909 AW963736 BE276064 BE380156 BE566181 BE567132 BE561903 AI205098 X52851 AW881897 BE616573 |
| | | BE298397 AA127519 R88890 BE616648 BE279162 BE559666 BE514563 BE312454 BE407312 BE276098 BE394047 |
| | | BE267326 BE276908 BE565569 BE385054 BE265740 BE409229 H44489 AA207193 AI525570 BE304612 BE396965 BE386123 |
| | | BE264758 BE266121 AA215851 BE295137 BE512683 BE388849 AA223494 BE263350 BE408879 BE386132 BE382906 |
| | | BE271789 BE539264 BE378507 BE567995 BE208471 Y00052 BE256484 BE260931 AL021395 BE260620 BE512892 AI906447 |
| | | BE615419 BE560786 BE279517 H46528 BE258084 BE535992 BE274774 BE274916 BE260585 BE563269 BE249941 |
| | | BE264782 BE408521 BE312750 BE559964 AA147099 T70141 AV661600 AA581711 AA186462 T69259 AA521058 AA563932 |
| | | BE397758 T68848 BE514531 H50191 AA641322 AI869370 AA653502 T70072 AW139957 AA069924 AI953984 C06029 |
| | | AA190399 AA617735 AA085181 AA506423 AI470746 C05982 BE077432 AW945661 BE272794 AA011065 AA922406 BE183113 |
| | | AA564289 AA374410 AI264729 AW957628 T68610 AA143328 AW381780 AW381880 AA135191 AA857453 AA112231 |
| | | AA011066 F36776 AW798236 H00395 BE272608 AW798123 AA532416 AI936732 BE183271 AA129828 AA741102 BE164382 |
| | | AW799104 AW997926 AA211136 AW601437 AW602618 H11277 AA083462 AA056516 AW364819 R01694 AA086193 |
| | | BE171120 BE093136 AW796864 AA663009 AW605266 AA247458 AW796905 BE171125 AW392363 BE171106 BE171110 |
| | | AW796810 AW796854 AW578648 AI221714 AW151499 AW937186 BE293542 BE249842 AW363879 AI701218 AW084288 |
| | | AA489447 AW798124 AW798256 AA658272 U46266 |
| 304983 | 25108_1 | AA329384 S49006 AW405735 AW404287 AW405848 AW405979 BE538908 H70947 AI814190 AA381896 AW582731 |
| | | AW975580 T94914 AW474500 AW869616 AW974125 AW946246 H44560 AW802184 AI538108 AW609912 AW865715 |
| | | BE007659 AA443571 AW376161 AW797783 AA713566 AW577973 AA745472 AW270172 AA910107 AA713717 AW364122 |
| | | AA533898 AI523353 AA715899 H44507 AW947263 AW390753 T89943 R66359 H44320 T64100 AA715879 R54661 AW579645 |
| | | H68597 AI499528 AI699010 AW605041 AW605043 AA236700 AI290767 H27472 AW079688 T69967 T87728 AW869468 |
| | | AW873389 AI250670 AA617786 AI566627 AW627605 AW302334 AA617836 AW793186 H16015 R83107 T85409 AA643479 |
| | | AA582449 AW794654 AI819995 AW865810 AW380149 AW793863 AI281629 T64021 AA501599 R49882 AA565066 R69406 |
| | | N23918 T59642 R54662 AW973199 R53006 AA401087 AA496563 AW934874 BE171294 AW793133 AI581595 AW605835 |
| | | AW605040 AW605055 AW605033 AW605058 AA291844 AW382472 AW605005 AA421881 AA513106 AA744190 AW386065 |
| | | AW613785 AW605829 AA580905 AW605027 AW364134 AA464027 BE012096 BE008565 AA809035 AA809154 AA911395 |
| | | AA713636 AA745324 AA826230 AW368498 AV646747 T89967 H61164 AI540661 AA721571 C02031 AA523762 AA523746 |
| | | AI287256 AW841164 AA962139 AW797014 AA515246 AW364140 H45842 T94267 T72111 R49771 T70151 H42664 R83656 |
| | | R83590 AW380297 AW380263 X95750 T57717 AW391436 AA293355 AW380185 X95749 X95748 X95747 AW796510 |
| | | AA318628 AI001043 AA744404 AA745567 AA650437 AA715526 AW392762 AA516228 |
| 321223 | 249691_1 | AA431366 AA490423 H96381 AA361614 H56121 AA354669 |
| 321240 | 2628_30 | X58799 AF071472 |
| 321286 | 236538_2 | BE245833 BE539992 AI380940 AW952644 AA535470 R84610 |
| 313476 | 83849_1 | AA010267 AA720674 AA010209 |
| 312854 | 467476_1 | AA837782 AA828713 H84206 AW979069 |
| 321304 | 115882_1 | BE260893 AA078319 R85057 AW803024 H85811 AA078293 |
| 321321 | 32811_1 | AB033072 AW163387 AW161566 T08007 AW161909 AW157331 |
| 321325 | 28266_1 | AB033100 AA347036 BE260325 AW961669 AL047207 AA347037 AI766894 AA601045 AI559897 AW139033 AW274622 |
| | | AW172884 AW089070 AA804340 AW798925 |
| 321354 | 116028_−2 | AA078493 |
| 321359 | 41877_1 | AW972301 AW474412 AJ227863 AA516297 AA641949 AW995029 |
| 320727 | 36759_63 | AW003360 AI971548 AA017585 X80306 X91133 AJ276100 X91132 AF064499 AF064495 AF063768 X83713 AW951310 |
| | | AW975565 AA721610 AA715972 |
| 320767 | 18895_2 | H54398 AA700844 AW895023 BE549308 AW834812 AA299525 AW957027 AA001789 BE219218 R73244 |
| 322012 | 22619_2 | AL137357 AW173064 AW662837 AI222955 AI138884 AW438842 AA883686 AA978208 AA653137 AA906477 AI150554 |
| | | BE295082 BE243550 |
| 306406 | AA971973 | |
| 306426 | AA975039 | |
| 306443 | AA976950 | |
| 306511 | AA988891 | |
| 320965 | 266751_1 | H18166 R86173 H19851 |
| 306531 | AA991423 | |
| 306546 | AA993109 | |
| 306549 | AA993796 | |
| 306558 | AA994743 | |
| 306590 | AI000246 | |
| 306591 | AI000248 | |
| 329464 | c_y_hs | |
| 336695 | CH22_4181FG_48_4_{—} | |
| 329496 | c10_p2 | |
| 336705 | CH22_4209FG_63_2_{—} | |
| 336717 | CH22_4234FG_81_1_{—} | |
| 306668 | AI004890 | |
| 306739 | AI028393 | |
| 306791 | AI042387 | |
| 329665 | c14_p2 | |
| 336883 | CH22_4625FG_322_2_{—} | |
| 336898 | CH22_4647FG_330_1 | |
| 308016 | AI445116 | |
| 308082 | AI473682 | |
| 338177 | CH22_6725FG_LINK_EM: AC00 | |
| 308092 | AI474896 | |
| 308097 | AI475411 | |
| 306801 | AI052653 | |
| 306809 | AI057134 | |
| 336908 | CH22_4670FG_343_2_{—} | |
| 336917 | CH22_4688FG_346_4_{—} | |
| 306839 | AI077385 | |
| 306866 | AI086683 | |
| 306881 | AI088695 | |
| 308115 | AI479071 | |
| 308127 | AI492187 | |
| 308138 | AI494446 | |
| 306919 | AI096832 | |
| 306930 | AI124518 | |
| 329824 | c14_p2 | |
| 329839 | c14_p2 | |
| 306975 | AI127042 | |
| 308258 | AI565612 | |
| 308282 | AI569456 | |
| 308311 | AI581855 | |
| 308382 | AI624301 | |
| 308385 | AI625428 | |
| 308406 | AI634885 | |
| 308449 | AI660854 | |
| 308465 | AI672480 | |
| 308517 | AI689279 | |
| 308539 | AI694191 | |
| 308544 | AI695133 | |
| 308548 | AI695484 | |
| 338648 | CH22_7413FG_LINK_EM: AC00 | |
| 308572 | AI707882 | |
| 338668 | CH22_7441FG_LINK_EM: AC00 | |
| 308582 | AI709056 | |
| 338679 | CH22_7453FG_LINK_EM: AC00 | |
| 308598 | AI719237 | |
| 338686 | CH22_7461FG_LINK_EM: AC00 | |
| 308601 | AI719930 | |
| 338703 | CH22_7483FG_LINK_EM: AC00 | |
| 338705 | CH22_7485FG_LINK_EM: AC00 | |
| 338725 | CH22_7521FG_LINK_EM: AC00 | |
| 338747 | CH22_7561FG_LINK_EM: AC00 | |
| 308676 | AI761036 | |
| 338770 | CH22_7593FG_LINK_EM: AC00 | |
| 308695 | AI763350 | |
| 308707 | AI769997 | |
| 308718 | AI798009 | |
| 308851 | AI829820 | |
| 308954 | AI868958 | |
| 308961 | AI870246 | |
| 333043 | CH22_269FG_70_4_LINK_EM: A | |
| 333054 | CH22_280FG_73_8_LINK_EM: A | |
| 308991 | AI879831 | |
| 303582 | 647662_1 | AA377444 AI458965 |
| 305018 | AA627127 | |
| 305030 | AA629988 | |
| 305046 | AA632201 | |
| 335131 | CH22_2463FG_497_15_LINK_E | |
| 335157 | CH22_2493FG_501_7_LINK_EM | |
| 305071 | AA640579 | |
| 335163 | CH22_2499FG_502_7_LINK_EM | |
| 305080 | AA641485 | |
| 335174 | CH22_2510FG_504_4_LINK_EM | |
| 305093 | AA642917 | |
| 305116 | AA649244 | |
| 335200 | CH22_2538FG_508_9_LINK_EM | |
| 335201 | CH22_2539FG_508_10_LINK_E | |
| 335217 | CH22_2556FG_512_3_LINK_EM | |
| 305134 | AA653159 | |
| 305145 | AA653589 | |
| 328015 | c_6_hs | |
| 328016 | c_6_hs | |
| 335234 | CH22_2573FG_515_3_LINK_EM | |
| 335236 | CH22_2577FG_515_8_LINK_EM | |
| 328025 | c_6_hs | |
| 335247 | CH22_2589FG_516_8_LINK_EM | |
| 305167 | AA663080 | |
| 305168 | AA663105 | |
| 328031 | c_6_hs | |
| 335262 | CH22_2604FG_520_3_LINK_EM | |
| 335265 | CH22_2607FG_521_1_LINK_EM | |
| 335266 | CH22_2608FG_521_2_LINK_EM | |
| 328053 | c_6_hs | |
| 305197 | AA666301 | |
| 335281 | CH22_2623FG_524_4_LINK_EM | |
| 335284 | CH22_2626FG_526_6_LINK_EM | |
| 328098 | c_6_hs | |
| 303929 | AW470753 | |
| 303933 | AW471472 | |
| 326806 | c20_hs | |
| 326808 | c20_hs | |
| 326857 | c20_hs | |
| 326874 | c20_hs | |
| 326876 | c20_hs | |
| 326884 | c20_hs | |
| 335311 | CH22_2654FG_532_4_LINK_EM | |
| 305234 | AA670431 | |
| 335320 | CH22_2664FG_534_7_LINK_EM | |
| 328109 | c_6_hs | |
| 335331 | CH22_2675FG_535_4_LINK_EM | |
| 335339 | CH22_2686FG_535_16_LINK_E | |
| 305259 | AA679225 | |
| 335340 | CH22_2687FG_535_17_LINK_E | |
| 335344 | CH22_2691FG_536_3_LINK_EM | |
| 335348 | CH22_2695FG_537_4_LINK_EM | |
| 335349 | CH22_2696FG_539_2_LINK_EM | |
| 305264 | AA679505 | |
| 328134 | c_6_hs | |
| 335371 | CH22_2720FG_543_9_LINK_EM | |
| 335377 | CH22_2726FG_543_17_LINK_E | |
| 328171 | C_6_hs | |
| 326942 | c21_hs | |
| 326943 | c21_hs | |
| 326957 | c21_hs | |
| 326981 | c21_hs | |
| 326997 | c21_hs | |
| 305335 | AA704235 | |
| 335421 | CH22_2772FG_551_1_LINK_EM | |
| 328221 | c_6_hs | |
| 328224 | c_6_hs | |
| 328228 | c_6_hs | |
| 335448 | CH22_2799FG_562_5_LINK_EM | |
| 305361 | AA708902 | |
| 335451 | CH22_2802FG_562_9_LINK_EM | |
| 328236 | c_6_hs | |
| 335455 | CH22_2806FG_562_15_LINK_E | |
| 328243 | c_6_hs | |
| 335468 | CH22_2819FG_567_4_LINK_EM | |
| 335470 | CH22_2821FG_568_3_LINK_EM | |
| 335482 | CH22_2834FG_570_11_LINK_E | |
| 335485 | CH22_2837FG_570_17_LINK_E | |
| 328271 | c_6_hs | |
| 328276 | c_7_hs | |
| 328277 | c_7_hs | |
| 328282 | c_7_hs | |
| 305403 | AA723748 | |
| 335517 | CH22_2872FG_571_34_LINK_E | |
| 328305 | c_7_hs | |
| 335523 | CH22_2878FG_572_3_LINK_EM | |
| 335527 | CH22_2882FG_572_7_LINK_EM | |
| 305443 | AA736653 | |
| 328314 | c_7_hs | |
| 335536 | CH22_2891FG_574_2_LINK_EM | |
| 305454 | AA738413 | |
| 328328 | c_7_hs | |
| 305464 | AA742425 | |
| 335565 | CH22_2921FG_579_1_LINK_EM | |
| 335566 | CH22_2922FG_580_1_LINK_EM | |
| 305486 | AA748889 | |
| 335585 | CH22_2943FG_581_24_LINK_E | |
| 335587 | CH22_2945FG_581_26_LINK_E | |
| 335593 | CH22_2951FG_581_32_LINK_E | |
| 335606 | CH22_2964FG_582_3_LINK_EM | |
| 305536 | AA770682 | |
| 305547 | AA773111 | |
| 335634 | CH22_2994FG_584_14_LINK_E | |
| 328420 | c_7_hs | |
| 328428 | c_7_hs | |
| 335651 | CH22_3011FG_590_2_LINK_EM | |
| 335652 | CH22_3012FG_590_3_LINK_EM | |
| 328436 | c_7_hs | |
| 328444 | c_7_hs | |
| 335667 | CH22_3027FG_590_18_LINK_E | |
| 328462 | c_7_hs | |
| 328467 | c_7_hs | |
| 335687 | CH22_3048FG_596_2_LINK_EM | |
| 335690 | CH22_3051FG_596_5_LINK_EM | |
| 328474 | c_7_hs | |
| 335692 | CH22_3053FG_596_7_LINK_EM | |
| 335693 | CH22_3054FG_596_8_LINK_EM | |
| 328484 | c_7_hs | |
| 335700 | CH22_3061FG_598_1_LINK_EM | |
| 305621 | AA789095 | |
| 305632 | AA805276 | |
| 335720 | CH22_3081FG_599_23_LINK_E | |
| 335721 | CH22_3082FG_599_24_LINK_E | |
| 328504 | c_7_hs | |
| 328506 | c_7_hs | |
| 328507 | c_7_hs | |
| 335733 | CH22_3095FG_601_3_LINK_EM | |
| 335739 | CH22_3102FG_601_10_LINK_E | |
| 335745 | CH22_3108FG_601_16_LINK_E | |
| 335747 | CH22_3111FG_601_20_LINK_E | |
| 335750 | CH22_3115FG_602_4_LINK_EM | |
| 335755 | CH22_3122FG_604_4_LINK_EM | |
| 328544 | c_7_hs | |
| 335768 | CH22_3137FG_607_2_LINK_EM | |
| 305686 | AA812726 | |
| 328552 | c_7_hs | |
| 335774 | CH22_3143FG_607_10_LINK_E | |
| 328557 | c_7_hs | |
| 328558 | c_7_hs | |
| 335777 | CH22_3146FG_607_13_LINK_E | |
| 305697 | AA814956 | |
| 335782 | CH22_3151FG_609_4_LINK_EM | |
| 335783 | CH22_3152FG_610_3_LINK_EM | |
| 328589 | c_7_hs | |
| 335787 | CH22_3156FG_611_3_LINK_EM | |
| 328570 | c_7_hs | |
| 328581 | c_7_hs | |
| 328582 | c_7_hs | |
| 328592 | c_7_hs | |
| 337011 | CH22_4876FG_427_6_{—} | |
| 337023 | CH22_4894FG_433_12_{—} | |
| 337032 | CH22_4910FG_438_3_{—} | |
| 337069 | CH22_4967FG_448_2_{—} | |
| 337092 | CH22_5016FG_465_12_{—} | |
| 337093 | CH22_5017FG_465_18_{—} | |
| 337094 | CH22_5018FG_465_19_{—} | |
| 337097 | CH22_5028FG_471_1_{—} | |
| 305700 | AA815428 | |
| 335806 | CH22_3178FG_616_8_LINK_EM | |
| 335817 | CH22_3189FG_618_5_LINK_EM | |
| 328607 | c_7_hs | |
| 335827 | CH22_3200FG_620_1_LINK_EM | |
| 335831 | CH22_3204FG_620_5_LINK_EM | |
| 335832 | CH22_3205FG_620_6_LINK_EM | |
| 328620 | c_7_hs | |
| 328624 | c_7_hs | |
| 328636 | c_7_hs | |
| 335863 | CH22_3238FG_629_8_LINK_EM | |
| 305782 | AA844730 | |
| 305787 | AA845035 | |
| 328662 | c_7_hs | |
| 335895 | CH22_3272FG_635_3_LINK_EM | |
| 337100 | CH22_5031FG_472_3_{—} | |
| 337114 | CH22_5060FG_494_17_{—} | |
| 337121 | CH22_5096FG_519_1_{—} | |
| 337132 | CH22_5112FG_526_3_{—} | |
| 337168 | CH22_5188FG_562_28_{—} | |
| 307085 | AI160868 | |
| 337170 | CH22_5190FG_564_1_{—} | |
| 337172 | CH22_5192FG_565_2_{—} | |
| 307090 | AI161024 | |
| 305803 | AA846052 | |
| 305808 | AA853958 | |
| 305816 | AA854776 | |
| 335902 | CH22_3279FG_635_10_LINK_E | |
| 335920 | CH22_3297FG_636_16_LINK_E | |
| 305841 | AA860348 | |
| 305867 | AA864572 | |
| 335956 | CH22_3334FG_647_3_LINK_DJ | |
| 305877 | AA865649 | |
| 335968 | CH22_3347FG_652_1_LINK_DJ | |
| 335971 | CH22_3350FG_652_4_LINK_DJ | |
| 335975 | CH22_3354FG_652_9_LINK_DJ | |
| 335980 | CH22_3360FG_653_2_LINK_DJ | |
| 328768 | c_7_hs | |
| 328770 | c_7_hs | |
| 335993 | CH22_3373FG_656_6_LINK_DJ | |
| 335998 | CH22_3379FG_656_16_LINK_D | |
| 335999 | CH22_3380FG_657_1_LINK_DJ | |
| 328791 | c_7_hs | |
| 337203 | CH22_5256FG_591_3_{—} | |
| 337204 | CH22_5261FG_595_1_{—} | |
| 307120 | AI184343 | |
| 307140 | AI185762 | |
| 307177 | AI188864 | |
| 305903 | AA873085 | |
| 305925 | AA877883 | |
| 328803 | c_7_hs | |
| 330002 | c16_p2 | |
| 328810 | c_7_hs | |
| 328820 | c_7_hs | |
| 330021 | c16_p2 | |
| 305967 | AA886428 | |
| 328835 | c_7_hs | |
| 305971 | AA886874 | |
| 328841 | c_7_hs | |
| 305984 | AA887654 | |
| 328851 | c_7_hs | |
| 328859 | c_7_hs | |
| 330057 | c17_p2 | |
| 330058 | c17_p2 | |
| 305999 | AA889603 | |
| 307215 | AI193189 | |
| 307262 | AI202100 | |
| 307318 | AI208577 | |
| 307380 | AI222985 | |
| 307433 | AI244895 | |
| 307437 | AI245683 | |
| 307556 | AI281651 | |
| 307558 | AI281998 | |
| 337645 | CH22_5960FG_LINK_EM: AC00 | |
| 337657 | CH22_5976FG_LINK_EM: AC00 | |
| 307574 | AI283549 | |
| 307588 | AI285535 | |
| 307592 | AI285739 | |
| 337685 | CH22_6020FG_LINK_EM: AC00 | |
| 337695 | CH22_6033FG_LINK_EM: AC00 | |
| 337697 | CH22_6037FG_LINK_EM: AC00 | |
| 307606 | AI290006 | |
| 307629 | AI300246 | |
| 307642 | AI302103 | |
| 307643 | AI302124 | |
| 307646 | AI302236 | |
| 307703 | AI318588 | |
| 307728 | AI335557 | |
| 307752 | AI339447 | |
| 307799 | AI351112 | |
| 309005 | AI884454 | |
| 307864 | AI367417 | |
| 307877 | AI368880 | |
| 307899 | AI380270 | |
| 309108 | AI925949 | |
| 309169 | AI949216 | |
| 309181 | AI951727 | |
| 307904 | AI381019 | |
| 307912 | AI382224 | |
| 307918 | AI383496 | |
| 307954 | AI419692 | |
| 307961 | AI421059 | |
| 307992 | AI434166 | |
| 309206 | AI961962 | |
| 309233 | AI971416 | |
| 309245 | AI972447 | |
| 309247 | AI972768 | |
| 309275 | AI989570 | |
| 309324 | AW015373 | |
| 309333 | AW025709 | |
| 309343 | AW028652 | |
| 309351 | AW057547 | |
| 309403 | AW082954 | |
| 309533 | AW151131 | |
| 309592 | AW172384 | |
| 325271 | c11_hs | |
| 325285 | c11_hs | |
| 325289 | c11_hs | |
| 309600 | AW182066 | |
| 309605 | AW182800 | |
| 309759 | AW268822 | |
| 309767 | AW271805 | |
| 309815 | AW292760 | |
| 309859 | AW298760 | |
| 302681 | 31257_1 | X97550 X97552 |
| 302683 | 31326_1 | X85153 T63701 |
| 309958 | AW444488 | |
| 309977 | AW451663 | |
| 309985 | AW452919 | |
| 302727 | 32493_1 | L10141 L10151 L10148 L09095 |
| 302747 | 32813_1 | AF062275 L03830 |
| 304022 | T02990 | |
| 304055 | R07994 | |
| 304056 | R08577 | |
| 304060 | T61464 | |
| 304125 | H40976 | |
| 304127 | H42981 | |
| 304134 | H54627 | |
| 304195 | N35382 | |
| 302952 | 39444_1 | AF103179 U82961 |
| 302996 | 41196_1 | AF054663 AF124197 R70292 |
| 304269 | AA069029 | |
| 304324 | AA137045 | |
| 304330 | AA157834 | |
| 304424 | AA293494 | |
| 304465 | AA421948 | |
| 304467 | AA424703 | |
| 304480 | AA430373 | |
| 304485 | AA434076 | |
| 304487 | AA434241 | |
| 304559 | AA488050 | |
| 304575 | AA496437 | |
| 304605 | AA513225 | |
| 304612 | AA514207 | |
| 304623 | AA521331 | |
| 304635 | AA523976 | |
| 304667 | AA535602 | |
| 304674 | AA541735 | |
| 304675 | AA541740 | |
| 304693 | AA554263 | |
| 304696 | AA554758 | |
| 304707 | AA564846 | |
| 304731 | AA576085 | |
| 304734 | AA576428 | |
| 304745 | AA577771 | |
| 304746 | AA577793 | |
| 306009 | AA894560 | |
| 306012 | AA896989 | |
| 306053 | AA905312 | |
| 306081 | AA908472 | |
| 306090 | AA908609 | |
| 304811 | AA584361 | |
| 304813 | AA584540 | |
| 304817 | AA584712 | |
| 304833 | AA586504 | |
| 304841 | AA587541 | |
| 304887 | AA599355 | |
| 306137 | AA916176 | |
| 306180 | AA922503 | |
| 306183 | AA922622 | |
| 306193 | AA923457 | |
| 304918 | AA602697 | |
| 304968 | AA614308 | |
| 306200 | AA926816 | |
| 306220 | AA928363 | |
| 306221 | AA928686 | |
| 306300 | AA937573 | |
| 320789 | 252516_1 | R78712 AA603646 R78713 |
| 306351 | AA961356 | |
| 330435 | 41165_1 | U63836 AW842139 X74956 U78550 AW840802 X74954 AW388241 AW842709 AF253321 X74955 X74370 AW363799 |
| | | BE073386 AI791962 AA587390 AW840865 |
| 330436 | 10605_34 | BE259039 W29128 AW410299 X72990 BE246492 NM_005243 X66899 AI909006 AW248151 AL031186 AA012966 BE273549 |
| | | BE311429 BE253102 Y07848 BE538102 BE256863 BE261240 BE312156 BE618412 BE257322 BE620446 AW806629 |
| | | AA376777 AA325384 BE256808 BE251039 BE257878 BE275352 AA357169 AW403562 AA204995 AA093259 W95953 |
| | | BE256279 BE336683 BE252465 BE251266 AA380754 BE294942 AA380941 AA380999 BE297164 BE249995 BE294719 |
| | | BE295372 AI270673 BE305132 BE563752 BE295357 AI525421 BE263980 AA057505 AA020915 BE266318 BE206948 |
| | | AI474020 BE296420 BE297374 BE408545 BE019366 BE407372 BE266180 BE279437 R58233 T19567 BE300738 AW381179 |
| | | AA357571 AW361285 AA436908 AA301019 AA301022 N20202 BE408777 BE548638 BE167415 AA071260 BE088429 |
| | | BE280092 W23117 T19568 R51681 AW402216 W22784 BE185607 AI457224 BE544120 AL134874 S72620 AA375079 D51319 |
| | | AW818280 BE514686 AW853024 BE563744 AA300469 T07592 BE622190 BE272834 W21781 BE315450 BE542367 BE393120 |
| | | AA988441 H55137 BE562296 BE622502 BE395960 AA329733 AA332348 AI768317 AA456866 AI497832 AW878437 AA857042 |
| | | U18018 BE621418 AI818790 AI949507 BE397693 AI885545 AI858854 AI355147 BE169028 S62138 AW732191 AA856891 |
| | | BE266060 X71427 BE268557 AF095890 AW001288 AI799634 AI623498 AA071346 BE547662 BE261446 AI564543 BE559759 |
| | | U35622 BE314249 BE264915 AI638591 AI538385 AW090025 BE384754 AI888689 AW778800 AI925273 AA075797 AW949130 |
| | | AV660275 AW438697 AI587137 AI524121 AA806249 AW628247 AA808241 AI244388 AI761125 AW117672 AA911782 |
| | | AI129250 AA654447 H55291 BE258050 BE206162 W95867 AA857187 AI871378 AI660103 AW103827 AI220929 AW149949 |
| | | BE465561 AI302857 AW168841 D82190 AW249814 AI623432 AI687358 AW951077 R51592 W60458 AI092863 AW474693 |
| | | D12765 AI911646 D82208 D82187 AW074031 AI358527 AW338497 AA970893 AW072573 AA205364 AI858886 AA012830 |
| | | AW148763 AI863056 AA548656 BE250325 AI016994 AI864005 BE046122 AI497746 C75340 R58896 D82141 AW168240 |
| | | C19048 AI741090 D29465 AI222365 AA948288 AI583522 AW572212 AI091290 AA582727 AA579897 AA570629 W60883 |
| | | AW516989 AL038160 AA577334 AI865872 AA994043 AA922583 AA464778 AA209178 AI829479 AI370235 BE246529 |
| | | AA384177 AA456255 AI699730 W60654 AL035744 AA862042 R32756 AI886886 AA993087 AI289479 AA627840 AA464184 |
| | | AI619503 R32755 AW075358 AI432315 AA457024 AA020865 R92132 AA454629 AA746059 AA454643 AA456240 AA826984 |
| | | BE163738 AI806470 AI991074 AI802560 AA587095 AA558714 AA968521 N87780 AI538246 N71794 AV661738 AI368903 |
| | | AA362570 AI989445 AI674962 S75762 BE245204 AA975296 D20123 AW005704 AA693328 AA582270 AI918474 AW205707 |
| | | AI696299 AA220990 AA101538 T29030 H27201 AW262526 AI610530 AA126840 AA126790 X92120 AW367868 BE299644 |
| | | BE299451 AA476561 BE300044 AA134363 BE295222 AA307504 N42337 AA319098 N39502 AW964461 N57241 BE299049 |
| | | N86332 R51156 AA085859 T75212 AA133939 AA147129 AA156161 BE543953 BE538848 AA133676 BE299745 AA135050 |
| | | AA218535 AW406401 AW411287 BE410528 C01410 NM_004083 BE314959 AA836413 AA085862 AW024370 AA471059 |
| | | AW467508 AA001025 AI828231 AA633221 T95517 AA147038 AA476447 AW027012 AW078627 BE513200 AI192297 |
| | | AA886279 AW081806 AA316185 AA010506 AI269929 W93139 AI682935 AA609555 AA378028 AI093877 AA999997 AA730698 |
| | | AI143923 AW575315 AA890550 AA494353 AW576601 AI796336 AA826130 AA609207 AI539618 AI088539 AI089090 AA825505 |
| | | AA632978 AA015892 AW204713 AA156495 AA824613 AA133630 N29826 AA527476 AI633352 T27908 AA134364 AA133940 |
| | | AW043601 H37775 AA772375 AA057871 AA047888 AA054225 H86568 AA001511 H25718 AW189507 AA165589 AA054433 |
| | | H85549 AA165486 AA058972 AA454911 AA464064 AA493802 AA428253 R85508 AW302469 AI611812 BE162582 F11073 |
| | | T95518 N26811 AI783929 H40669 AW611745 AI658803 R51042 R45276 AA528386 AA782875 AW880218 AL138391 AA314536 |
| | | AW949338 AA149466 AA149552 AI346513 AA216776 BE349131 AW007654 AI141803 AA622688 AI185131 AW057635 |
| | | AA101539 AA627986 H27202 AI536847 W93084 AI973148 AI246788 AW572108 AI469414 AA454835 AA612707 AA430746 |
| | | AI084991 AA010400 AA856636 AA463928 AI248310 R07170 AA834033 D12244 AI655670 AA054350 AA639480 AI702067 |
| | | AI475389 |
| 330527 | 14558_−15 | S77356 |
| 330833 | 103550_1 | AA046804 AA046821 |
| 330855 | 111881_1 | AA070316 AA079318 |
| 332099 | genbanK_AA608983 | AA608983 |
| 332240 | genbank_N54803 | N54803 |
| Table 1-20A, shows the accession numbers for those pkeys lacking unigeneID's for Tables 1-20. For each probeset we have listed the gene cluster number from which the oligonucleotides were designed. Gene clusters were compiled using sequences derived from Genbank ESTs and mRNAs. These sequences were clustered based on sequence similarity | | |
| # using Clustering and Alignment Tools (DoubleTwist, Oakland California). The Genbank accession numbers for sequences comprising each cluster are listed in the “Accession”column. | | |

Pkey: Unique Eos probeset identifier number
CAT number: Gene cluster number
Accession: Genbank accession numbers

TABLE 1-20B

| Pkey | Ref | Strand | Nt_position |
|---|---|---|---|
| 332792 | Dunham, I. et.al. | Plus | 73381-73768 |
| 332843 | Dunham, I. et.al. | Plus | 1142859-1143494 |
| 332909 | Dunham, I. et.al. | Plus | 1946582-1946735 |
| 332920 | Dunham, I. et.al. | Plus | 2007562-2007785 |
| 332947 | Dunham, I. et.al. | Plus | 2431726-2432006 |
| 332949 | Dunham, I. et.al. | Plus | 2436245-2436348 |
| 332958 | Dunham, I. et.al. | Plus | 2516164-2516310 |
| 332992 | Dunham, I. et.al. | Plus | 2699997-2701093 |
| 332993 | Dunham, I. et.al. | Plus | 2701550-2701685 |
| 333004 | Dunham, I. et.al. | Plus | 2759056-2759165 |
| 333006 | Dunham, I. et.al. | Plus | 2762853-2762953 |
| 333007 | Dunham, I. et.al. | Plus | 2763569-2763709 |
| 333132 | Dunham, I. et.al. | Plus | 3358040-3358153 |
| 333133 | Dunham, I. et.al. | Plus | 3360058-3360195 |
| 333139 | Dunham, I. et.al. | Plus | 3369495-3369571 |
| 333152 | Dunham, I. et.al. | Plus | 3612171-3612354 |
| 333205 | Dunham, I. et.al. | Plus | 3942727-3943009 |
| 333221 | Dunham, I. et.al. | Plus | 3978070-3978187 |
| 333225 | Dunham, I. et.al. | Plus | 3992229-3992386 |
| 333245 | Dunham, I. et.al. | Plus | 4157587-4157668 |
| 333248 | Dunham, I. et.al. | Plus | 4162041-4162139 |
| 333261 | Dunham, I. et.al. | Plus | 4336597-4337752 |
| 333272 | Dunham, I. et.al. | Plus | 4381561-4382212 |
| 333281 | Dunham, I. et.al. | Plus | 4506230-4506342 |
| 333283 | Dunham, I. et.al. | Plus | 4514226-4514360 |
| 333288 | Dunham, I. et.al. | Plus | 4516841-4516939 |
| 333298 | Dunham, I. et.al. | Plus | 4581537-4581947 |
| 333306 | Dunham, I. et.al. | Plus | 5396233-5396310 |
| 333382 | Dunham, I. et.al. | Plus | 4905796-4905913 |
| 333403 | Dunham, I. et.al. | Plus | 4925140-4925256 |
| 333420 | Dunham, I. et.al. | Plus | 4954302-4954465 |
| 333428 | Dunham, I. et.al. | Plus | 4973869-4974007 |
| 333464 | Dunham, I. et.al. | Plus | 5210762-5211300 |
| 333465 | Dunham, I. et.al. | Plus | 5211385-5211858 |
| 333488 | Dunham, I. et.al. | Plus | 5396233-5396310 |
| 333515 | Dunham, I. et.al. | Plus | 5564299-5564851 |
| 333520 | Dunham, I. et.al. | Plus | 5686133-5586296 |
| 333566 | Dunham, I. et.al. | Plus | 5954226-5954473 |
| 333567 | Dunham, I. et.al. | Plus | 5959139-5959515 |
| 333571 | Dunham, I. et.al. | Plus | 6007916-6008058 |
| 333572 | Dunham, I. et.al. | Plus | 6026896-6027189 |
| 333576 | Dunham, I. et.al. | Plus | 6090345-6090721 |
| 333577 | Dunham, I. et.al. | Plus | 6123950-6124281 |
| 333580 | Dunham, I. et.al. | Plus | 6142935-6143145 |
| 333587 | Dunham, I. et.al. | Plus | 6250599-6250966 |
| 333588 | Dunham, I. et.al. | Plus | 6255445-6255779 |
| 333591 | Dunham, I. et.al. | Plus | 6285884-6286251 |
| 333592 | Dunham, I. et.al. | Plus | 6297731-6297976 |
| 333593 | Dunham, I. et.al. | Plus | 6304132-6304428 |
| 333594 | Dunham, I. et.al. | Plus | 6308990-6309450 |
| 333599 | Dunham, I. et.al. | Plus | 6337885-6338255 |
| 333600 | Dunham, I. et.al. | Plus | 6355629-6355925 |
| 333601 | Dunham, I. et.al. | Plus | 6360075-6360442 |
| 333607 | Dunham, I. et.al. | Plus | 6504431-6504690 |
| 333608 | Dunham, I. et.al. | Plus | 6510834-6511130 |
| 333619 | Dunham, I. et.al. | Plus | 6562799-6562926 |
| 333623 | Dunham, I. et.al. | Plus | 6584559-6584956 |
| 333625 | Dunham, I. et.al. | Plus | 6603020-6603310 |
| 333626 | Dunham, I. et.al. | Plus | 6614174-6614467 |
| 333627 | Dunham, I. et.al. | Plus | 6620584-6620903 |
| 333628 | Dunham, I. et.al. | Plus | 6629004-6629233 |
| 333629 | Dunham, I. et.al. | Plus | 6636915-6637205 |
| 333631 | Dunham, I. et.al. | Plus | 6650904-6651011 |
| 333632 | Dunham, I. et.al. | Plus | 6651520-6651658 |
| 333635 | Dunham, I. et.al. | Plus | 6663683-6663973 |
| 333637 | Dunham, I. et.al. | Plus | 6674968-6675134 |
| 333640 | Dunham, I. et.al. | Plus | 6688350-6688624 |
| 333642 | Dunham, I. et.al. | Plus | 6708760-6709139 |
| 333643 | Dunham, I. et.al. | Plus | 6728053-6728343 |
| 333646 | Dunham, I. et.al. | Plus | 6739110-6739379 |
| 333647 | Dunham, I. et.al. | Plus | 6772502-6772779 |
| 333648 | Dunham, I. et.al. | Plus | 6787465-6787782 |
| 333650 | Dunham, I. et.al. | Plus | 6796852-6797128 |
| 333652 | Dunham, I. et.al. | Plus | 6809455-6809573 |
| 333653 | Dunham, I. et.al. | Plus | 6811130-6811392 |
| 333654 | Dunham, I. et.al. | Plus | 6816731-6816993 |
| 333656 | Dunham, I. et.al. | Plus | 6822087-6822406 |
| 333657 | Dunham, I. et.al. | Plus | 6831369-6831445 |
| 333658 | Dunham, I. et.al. | Plus | 6835282-6835474 |
| 333668 | Dunham, I. et.al. | Plus | 7011009-7011223 |
| 333670 | Dunham, I. et.al. | Plus | 7027945-7028181 |
| 333680 | Dunham, I. et.al. | Plus | 7071730-7071794 |
| 333682 | Dunham, I. et.al. | Plus | 7076641-7076760 |
| 333698 | Dunham, I. et.al. | Plus | 7205279-7205383 |
| 333710 | Dunham, I. et.al. | Plus | 7230314-7230476 |
| 333717 | Dunham, I. et.al. | Plus | 7308714-7308815 |
| 333727 | Dunham, I. et.al. | Plus | 7373219-7373311 |
| 333785 | Dunham, I. et.al. | Plus | 7775317-7775415 |
| 333791 | Dunham, I. et.al. | Plus | 7795972-7796082 |
| 333859 | Dunham, I. et.al. | Plus | 8041203-8041359 |
| 333875 | Dunham, I. et.al. | Plus | 8135505-8136179 |
| 333879 | Dunham, I. et.al. | Plus | 8146919-8147062 |
| 333891 | Dunham, I. et.al. | Plus | 8156437-8156709 |
| 333918 | Dunham, I. et.al. | Plus | 8307124-8307215 |
| 333929 | Dunham, I. et.al. | Plus | 8479486-8479580 |
| 333932 | Dunham, I. et.al. | Plus | 8489124-8489205 |
| 333983 | Dunham, I. et.al. | Plus | 8813593-8813668 |
| 333987 | Dunham, I. et.al. | Plus | 8824245-8824376 |
| 333997 | Dunham, I. et.al. | Plus | 8866668-8867255 |
| 334010 | Dunham, I. et.al. | Plus | 8996696-8998236 |
| 334015 | Dunham, I. et.al. | Plus | 9055452-9055595 |
| 334017 | Dunham, I. et.al. | Plus | 9139516-9139634 |
| 334026 | Dunham, I. et.al. | Plus | 9196549-9196681 |
| 334030 | Dunham, I. et.al. | Plus | 9288463-9288782 |
| 334044 | Dunham, I. et.al. | Plus | 9373898-9374065 |
| 334047 | Dunham, I. et.al. | Plus | 9428152-9428211 |
| 334055 | Dunham, I. et.al. | Plus | 9662077-9662270 |
| 334063 | Dunham, I. et.al. | Plus | 9731991-9732085 |
| 334066 | Dunham, I. et.al. | Plus | 9739568-9739680 |
| 334068 | Dunham, I. et.al. | Plus | 9746279-9746477 |
| 334076 | Dunham, I. et.al. | Plus | 9801613-9801693 |
| 334091 | Dunham, I. et.al. | Plus | 9872327-9872527 |
| 334106 | Dunham, I. et.al. | Plus | 10261155-10261841 |
| 334109 | Dunham, I. et.al. | Plus | 10267679-10267864 |
| 334111 | Dunham, I. et.al. | Plus | 10279365-10279531 |
| 334115 | Dunham, I. et.al. | Plus | 10316414-10316608 |
| 334118 | Dunham, I. et.al. | Plus | 10344273-10344384 |
| 334120 | Dunham, I. et.al. | Plus | 10402389-10403196 |
| 334135 | Dunham, I. et.al. | Plus | 10457085-10457183 |
| 334235 | Dunham, I. et.al. | Plus | 12983601-12983703 |
| 334239 | Dunham, I. et.al. | Plus | 13056569-13056693 |
| 334244 | Dunham, I. et.al. | Plus | 13159198-13159302 |
| 334257 | Dunham, I. et.al. | Plus | 13213243-13213429 |
| 334260 | Dunham, I. et.al. | Plus | 13223819-13223968 |
| 334298 | Dunham, I. et.al. | Plus | 13424763-13425914 |
| 334342 | Dunham, I. et.al. | Plus | 13646844-13646980 |
| 334354 | Dunham, I. et.al. | Plus | 13702598-13702747 |
| 334430 | Dunham, I. et.al. | Plus | 14269664-14270102 |
| 334435 | Dunham, I. et.al. | Plus | 14275597-14275689 |
| 334451 | Dunham, I. et.al. | Plus | 14315572-14315741 |
| 334504 | Dunham, I. et.al. | Plus | 14510206-14510398 |
| 334510 | Dunham, I. et.al. | Plus | 14522303-14522418 |
| 334518 | Dunham, I. et.al. | Plus | 14630584-14630669 |
| 334525 | Dunham, I. et.al. | Plus | 14781066-14781137 |
| 334528 | Dunham, I. et.al. | Plus | 14787558-14787675 |
| 334529 | Dunham, I. et.al. | Plus | 14788825-14788971 |
| 334565 | Dunham, I. et.al. | Plus | 14989033-14989353 |
| 334568 | Dunham, I. et.al. | Plus | 14992698-14993210 |
| 334590 | Dunham, I. et.al. | Plus | 15033247-15033753 |
| 334612 | Dunham, I. et.al. | Plus | 15170470-15170535 |
| 334626 | Dunham, I. et.al. | Plus | 15299954-15300077 |
| 334661 | Dunham, I. et.al. | Plus | 15477716-15477786 |
| 334664 | Dunham, I. et.al. | Plus | 15501941-15502119 |
| 334666 | Dunham, I. et.al. | Plus | 15504203-15504279 |
| 334676 | Dunham, I. et.al. | Plus | 15516334-15516412 |
| 334677 | Dunham, I. et.al. | Plus | 15517449-15517560 |
| 334719 | Dunham, I. et.al. | Plus | 15778859-15779026 |
| 334730 | Dunham, I. et.al. | Plus | 15967830-15967934 |
| 334797 | Dunham, I. et.al. | Plus | 16383231-16383458 |
| 334819 | Dunham, I. et.al. | Plus | 16758514-16758711 |
| 334855 | Dunham, I. et.al. | Plus | 18402556-18402735 |
| 334900 | Dunham, I. et.al. | Plus | 19315678-19315743 |
| 334906 | Dunham, I. et.al. | Plus | 19323493-19323590 |
| 334907 | Dunham, I. et.al. | Plus | 19336829-19336938 |
| 334914 | Dunham, I. et.al. | Plus | 19495158-19495275 |
| 334915 | Dunham, I. et.al. | Plus | 19505267-19506101 |
| 334935 | Dunham, I. et.al. | Plus | 20108247-20108373 |
| 335019 | Dunham, I. et.al. | Plus | 20689525-20689582 |
| 335036 | Dunham, I. et.al. | Plus | 20755250-20756375 |
| 335049 | Dunham, I. et.al. | Plus | 20877184-20877309 |
| 335081 | Dunham, I. et.al. | Plus | 21113871-21113937 |
| 335131 | Dunham, I. et.al. | Plus | 21449834-21450003 |
| 335157 | Dunham, I. et.al. | Plus | 21543302-21544341 |
| 335163 | Dunham, I. et.al. | Plus | 21583508-21583655 |
| 335174 | Dunham, I. et.al. | Plus | 21631301-21631447 |
| 335180 | Dunham, I. et.al. | Plus | 21641226-21641603 |
| 335188 | Dunham, I. et.al. | Plus | 21669118-21669328 |
| 335189 | Dunham, I. et.al. | Plus | 21673403-21673472 |
| 335193 | Dunham, I. et.al. | Plus | 21692208-21692362 |
| 335200 | Dunham, I. et.al. | Plus | 21743499-21743881 |
| 335201 | Dunham, I. et.al. | Plus | 21745249-21745664 |
| 335284 | Dunham, I. et.al. | Plus | 22272583-22272712 |
| 335311 | Dunham, I. et.al. | Plus | 22501602-22501676 |
| 335320 | Dunham, I. et.al. | Plus | 22542132-22542246 |
| 335371 | Dunham, I. et.al. | Plus | 22849529-22849808 |
| 335377 | Dunham, I. et.al. | Plus | 22869909-22870188 |
| 335421 | Dunham, I. et.al. | Plus | 23250433-23250498 |
| 335448 | Dunham, I. et.al. | Plus | 23477532-23477831 |
| 335451 | Dunham, I. et.al. | Plus | 23480611-23480711 |
| 335455 | Dunham, I. et.al. | Plus | 23486127-23486216 |
| 335468 | Dunham, I. et.al. | Plus | 23787246-23787367 |
| 335470 | Dunham, I. et.al. | Plus | 23812877-23813365 |
| 335482 | Dunham, I. et.al. | Plus | 24087956-24088114 |
| 335485 | Dunham, I. et.al. | Plus | 24098953-24099123 |
| 335517 | Dunham, I. et.al. | Plus | 24226461-24226531 |
| 335536 | Dunham, I. et.al. | Plus | 24498783-24499762 |
| 335565 | Dunham, I. et.al. | Plus | 24881886-24882470 |
| 335585 | Dunham, I. et.al. | Plus | 24988240-24988405 |
| 335587 | Dunham, I. et.al. | Plus | 24991457-24991618 |
| 335593 | Dunham, I. et.al. | Plus | 25037253-25037370 |
| 335634 | Dunham, I. et.al. | Plus | 25163837-25164042 |
| 335651 | Dunham, I. et.al. | Plus | 25317560-25317696 |
| 335652 | Dunham, I. et.al. | Plus | 25325625-25325883 |
| 335667 | Dunham, I. et.al. | Plus | 25345735-25345856 |
| 335687 | Dunham, I. et.al. | Plus | 25445952-25446064 |
| 335690 | Dunham, I. et.al. | Plus | 25455442-25455625 |
| 335692 | Dunham, I. et.al. | Plus | 25468557-25468725 |
| 335693 | Dunham, I. et.al. | Plus | 25471363-25471524 |
| 335700 | Dunham, I. et.al. | Plus | 25512222-25512890 |
| 335720 | Dunham, I. et.al. | Plus | 25596542-25596646 |
| 335721 | Dunham, I. et.al. | Plus | 25599118-25599228 |
| 335733 | Dunham, I. et.al. | Plus | 25686486-25686626 |
| 335739 | Dunham, I. et.al. | Plus | 25698550-25698826 |
| 335745 | Dunham, I. et.al. | Plus | 25718164-25718332 |
| 335747 | Dunham, I. et.al. | Plus | 25723304-25723494 |
| 335783 | Dunham, I. et.al. | Plus | 25941882-25942040 |
| 335968 | Dunham, I. et.al. | Plus | 27743843-27744029 |
| 335971 | Dunham, I. et.al. | Plus | 27752808-27753017 |
| 335975 | Dunham, I. et.al. | Plus | 27801321-27801391 |
| 335993 | Dunham, I. et.al. | Plus | 28006396-28006571 |
| 335998 | Dunham, I. et.al. | Plus | 28026247-28026356 |
| 336011 | Dunham, I. et.al. | Plus | 28570760-28570900 |
| 336035 | Dunham, I. et.al. | Plus | 29016748-29017410 |
| 336049 | Dunham, I. et.al. | Plus | 29124451-29124606 |
| 336055 | Dunham, I. et.al. | Plus | 29166352-29166573 |
| 336072 | Dunham, I. et.al. | Plus | 29269409-29269575 |
| 336075 | Dunham, I. et.al. | Plus | 29343938-29344050 |
| 336081 | Dunham, I. et.al. | Plus | 29372270-29372407 |
| 336083 | Dunham, I. et.al. | Plus | 29379738-29379881 |
| 336084 | Dunham, I. et.al. | Plus | 29380664-29380792 |
| 336086 | Dunham, I. et.al. | Plus | 29385594-29385713 |
| 336087 | Dunham, I. et.al. | Plus | 29387122-29387253 |
| 336088 | Dunham, I. et.al. | Plus | 29389001-29389124 |
| 336089 | Dunham, I. et.al. | Plus | 29401421-29401625 |
| 336090 | Dunham, I. et.al. | Plus | 29413020-29413162 |
| 336091 | Dunham, I. et.al. | Plus | 29415486-29415617 |
| 336092 | Dunham, I. et.al. | Plus | 29438816-29438948 |
| 336094 | Dunham, I. et.al. | Plus | 29567934-29568099 |
| 336102 | Dunham, I. et.al. | Plus | 29681500-29681687 |
| 336124 | Dunham, I. et.al. | Plus | 30053441-30053500 |
| 336125 | Dunham, I. et.al. | Plus | 30056541-30056622 |
| 336127 | Dunham, I. et.al. | Plus | 30058870-30059029 |
| 336128 | Dunham, I. et.al. | Plus | 30059294-30059367 |
| 336171 | Dunham, I. et.al. | Plus | 30223877-30224048 |
| 336214 | Dunham, I. et.al. | Plus | 30561988-30562044 |
| 336306 | Dunham, I. et.al. | Plus | 33172949-33173281 |
| 336316 | Dunham, I. et.al. | Plus | 33338776-33338919 |
| 336333 | Dunham, I. et.al. | Plus | 33673947-33677345 |
| 336387 | Dunham, I. et.al. | Plus | 34013939-34014124 |
| 336442 | Dunham, I. et.al. | Plus | 34187937-34188081 |
| 336481 | Dunham, I. et.al. | Plus | 34231611-34231720 |
| 336489 | Dunham, I. et.al. | Plus | 34239511-34239619 |
| 336498 | Dunham, I. et.al. | Plus | 34267340-34267424 |
| 336502 | Dunham, I. et.al. | Plus | 34268953-34269083 |
| 336516 | Dunham, I. et.al. | Plus | 34296068-34296249 |
| 336548 | Dunham, I. et.al. | Plus | 34353881-34354826 |
| 336590 | Dunham, I. et.al. | Plus | 2569428-2570275 |
| 336593 | Dunham, I. et.al. | Plus | 4617967-4618783 |
| 336606 | Dunham, I. et.al. | Plus | 16170215-16170415 |
| 336623 | Dunham, I. et.al. | Plus | 167287-167616 |
| 336632 | Dunham, I. et.al. | Plus | 983890-985529 |
| 336633 | Dunham, I. et.al. | Plus | 985591-986221 |
| 336634 | Dunham, I. et.al. | Plus | 986296-986670 |
| 336635 | Dunham, I. et.al. | Plus | 987908-988364 |
| 336637 | Dunham, I. et.al. | Plus | 989276-990813 |
| 336638 | Dunham, I. et.al. | Plus | 991906-993240 |
| 336654 | Dunham, I. et.al. | Plus | 1588782-1588858 |
| 336665 | Dunham, I. et.al. | Plus | 2007241-2007363 |
| 336695 | Dunham, I. et.al. | Plus | 2464609-2464739 |
| 336705 | Dunham, I. et.al. | Plus | 2817668-2817723 |
| 336751 | Dunham, I. et.al. | Plus | 4499629-4499739 |
| 336753 | Dunham, I. et.al. | Plus | 4509509-4509601 |
| 336757 | Dunham, I. et.al. | Plus | 4541167-4541466 |
| 336759 | Dunham, I. et.al. | Plus | 4560526-4560699 |
| 336808 | Dunham, I. et.al. | Plus | 6251639-6251761 |
| 336813 | Dunham, I. et.al. | Plus | 6436416-6436719 |
| 336827 | Dunham, I. et.al. | Plus | 6773762-6774057 |
| 336833 | Dunham, I. et.al. | Plus | 6856506-6856634 |
| 336836 | Dunham, I. et.al. | Plus | 7077262-7077326 |
| 336878 | Dunham, I. et.al. | Plus | 9200300-9200399 |
| 336898 | Dunham, I. et.al. | Plus | 10138365-10138458 |
| 336917 | Dunham, I. et.al. | Plus | 11228329-11228403 |
| 336924 | Dunham, I. et.al. | Plus | 11525273-11525527 |
| 336934 | Dunham, I. et.al. | Plus | 11854506-11854792 |
| 336958 | Dunham, I. et.al. | Plus | 13203550-13203973 |
| 336968 | Dunham, I. et.al. | Plus | 13660250-13660530 |
| 336986 | Dunham, I. et.al. | Plus | 14785058-14785225 |
| 337032 | Dunham, I. et.al. | Plus | 16949229-16949306 |
| 337121 | Dunham, I. et.al. | Plus | 22050289-22050370 |
| 337132 | Dunham, I. et.al. | Plus | 22255234-22255437 |
| 337168 | Dunham, I. et.al. | Plus | 23503596-23504240 |
| 337170 | Dunham, I. et.al. | Plus | 23533763-23534008 |
| 337172 | Dunham, I. et.al. | Plus | 23608358-23608526 |
| 337218 | Dunham, I. et.al. | Plus | 26126466-26126565 |
| 337219 | Dunham, I. et.al. | Plus | 26129629-26130053 |
| 337236 | Dunham, I. et.al. | Plus | 27114318-27114571 |
| 337238 | Dunham, I. et.al. | Plus | 27141465-27141776 |
| 337259 | Dunham, I. et.al. | Plus | 27674026-27674142 |
| 337291 | Dunham, I. et.al. | Plus | 28823308-28823412 |
| 337298 | Dunham, I. et.al. | Plus | 29002509-29002806 |
| 337304 | Dunham, I. et.al. | Plus | 29131691-29131823 |
| 337333 | Dunham, I. et.al. | Plus | 30281383-30281673 |
| 337338 | Dunham, I. et.al. | Plus | 30427592-30427775 |
| 337345 | Dunham, I. et.al. | Plus | 30649387-30649467 |
| 337355 | Dunham, I. et.al. | Plus | 30832838-30833042 |
| 337363 | Dunham, I. et.al. | Plus | 30942712-30942840 |
| 337396 | Dunham, I. et.al. | Plus | 31585902-31586067 |
| 337402 | Dunham, I. et.al. | Plus | 31691134-31691482 |
| 337403 | Dunham, I. et.al. | Plus | 31692319-31692611 |
| 337425 | Dunham, I. et.al. | Plus | 32072241-32072334 |
| 337426 | Dunham, I. et.al. | Plus | 32076190-32076349 |
| 337427 | Dunham, I. et.al. | Plus | 32078958-32079076 |
| 337435 | Dunham, I. et.al. | Plus | 32255716-32255844 |
| 337484 | Dunham, I. et.al. | Plus | 33238033-33238176 |
| 337489 | Dunham, I. et.al. | Plus | 33295724-33295872 |
| 337503 | Dunham, I. et.al. | Plus | 33385583-33385857 |
| 337504 | Dunham, I. et.al. | Plus | 33386053-33386236 |
| 337522 | Dunham, I. et.al. | Plus | 33963188-33963979 |
| 337544 | Dunham, I. et.al. | Plus | 34268412-34268495 |
| 337548 | Dunham, I. et.al. | Plus | 34472882-34472957 |
| 337562 | Dunham, I. et.al. | Plus | 278105-278281 |
| 337564 | Dunham, I. et.al. | Plus | 285643-285788 |
| 337586 | Dunham, I. et.al. | Plus | 952102-952250 |
| 337628 | Dunham, I. et.al. | Plus | 2013838-2014032 |
| 337657 | Dunham, I. et.al. | Plus | 2844851-2845055 |
| 337685 | Dunham, I. et.al. | Plus | 3547161-3547245 |
| 337695 | Dunham, I. et.al. | Plus | 3609670-3609808 |
| 337697 | Dunham, I. et.al. | Plus | 3644971-3645195 |
| 337756 | Dunham, I. et.al. | Plus | 3975015-3975172 |
| 337781 | Dunham, I. et.al. | Plus | 4116049-4116265 |
| 337797 | Dunham, I. et.al. | Plus | 4325712-4325811 |
| 337835 | Dunham, I. et.al. | Plus | 4792299-4792423 |
| 337843 | Dunham, I. et.al. | Plus | 4984966-4985082 |
| 337865 | Dunham, I. et.al. | Plus | 5377705-5377861 |
| 337870 | Dunham, I. et.al. | Plus | 5442516-5442636 |
| 337904 | Dunham, I. et.al. | Plus | 5685819-5686012 |
| 337918 | Dunham, I. et.al. | Plus | 6031241-6031358 |
| 337930 | Dunham, I. et.al. | Plus | 6393566-6393692 |
| 337947 | Dunham, I. et.al. | Plus | 6675643-6675781 |
| 337951 | Dunham, I. et.al. | Plus | 6766321-6766382 |
| 337954 | Dunham, I. et.al. | Plus | 6831483-6831620 |
| 337964 | Dunham, I. et.al. | Plus | 7032720-7032802 |
| 338043 | Dunham, I. et.al. | Plus | 8222138-8222430 |
| 338054 | Dunham, I. et.al. | Plus | 8429258-8429382 |
| 338062 | Dunham, I. et.al. | Plus | 8578852-4579012 |
| 338080 | Dunham, I. et.al. | Plus | 9212114-9212220 |
| 338090 | Dunham, I. et.al. | Plus | 9488619-9488858 |
| 338096 | Dunham, I. et.al. | Plus | 9698397-9698469 |
| 338120 | Dunham, I. et.al. | Plus | 10765673-10765820 |
| 338123 | Dunham, I. et.al. | Plus | 10615824-10815886 |
| 338124 | Dunham, I. et.al. | Plus | 10860311-10860471 |
| 338177 | Dunham, I. et.al. | Plus | 12781530-12781724 |
| 338239 | Dunham, I. et.al. | Plus | 14669918-14670016 |
| 338278 | Dunham, I. et.al. | Plus | 16168714-16168840 |
| 338280 | Dunham, I. et.al. | Plus | 16171884-16172039 |
| 338326 | Dunham, I. et.al. | Plus | 17358806-17358964 |
| 338336 | Dunham, I. et.al. | Plus | 17453324-17453448 |
| 338344 | Dunham, I. et.al. | Plus | 17780512-17780623 |
| 338411 | Dunham, I. et.al. | Plus | 19299273-19299522 |
| 338412 | Dunham, I. et.al. | Plus | 19340661-19340737 |
| 338491 | Dunham, I. et.al. | Plus | 21168430-21168556 |
| 338514 | Dunham, I. et.al. | Plus | 21379420-21379655 |
| 338516 | Dunham, I. et.al. | Plus | 21382180-21382383 |
| 338521 | Dunham, I. et.al. | Plus | 21471358-21471476 |
| 338522 | Dunham, I. et.al. | Plus | 21471617-21471711 |
| 338526 | Dunham, I. et.al. | Plus | 21551136-21551250 |
| 338527 | Dunham, I. et.al. | Plus | 21552237-21553280 |
| 338529 | Dunham, I. et.al. | Plus | 21635616-21635867 |
| 338579 | Dunham, I. et.al. | Plus | 22774968-22775132 |
| 338596 | Dunham, I. et.al. | Plus | 23078273-23078348 |
| 338648 | Dunham, I. et.al. | Plus | 24091259-24091434 |
| 338679 | Dunham, I. et.al. | Plus | 24684836-24684940 |
| 338703 | Dunham, I. et.al. | Plus | 25219881-25220046 |
| 338705 | Dunham, I. et.al. | Plus | 25231397-25231551 |
| 338747 | Dunham, I. et.al. | Plus | 26336372-26336460 |
| 338770 | Dunham, I. et.al. | Plus | 26784513-26784714 |
| 338809 | Dunham, I. et.al. | Plus | 27271920-27272000 |
| 338830 | Dunham, I. et.al. | Plus | 27759923-27760055 |
| 338880 | Dunham, I. et.al. | Plus | 28420713-28420826 |
| 338895 | Dunham, I. et.al. | Plus | 28598893-28599135 |
| 338896 | Dunham, I. et.al. | Plus | 28618487-28618552 |
| 338911 | Dunham, I. et.al. | Plus | 28804184-28804445 |
| 338925 | Dunham, I. et.al. | Plus | 28883892-28884036 |
| 338952 | Dunham, I. et.al. | Plus | 29418831-29418968 |
| 338959 | Dunham, I. et.al. | Plus | 29478431-29478589 |
| 338970 | Dunham, I. et.al. | Plus | 29645468-29645613 |
| 339081 | Dunham, I. et.al. | Plus | 30927871-30928080 |
| 339116 | Dunham, I. et.al. | Plus | 31462304-31462382 |
| 339164 | Dunham, I. et.al. | Plus | 32207441-32207802 |
| 339246 | Dunham, I. et.al. | Plus | 32844518-32844586 |
| 339271 | Dunham, I. et.al. | Plus | 32999935-33000078 |
| 339304 | Dunham, I. et.al. | Plus | 33323712-33323821 |
| 339309 | Dunham, I. et.al. | Plus | 33366900-33367198 |
| 339312 | Dunham, I. et.al. | Plus | 33378992-33379172 |
| 339356 | Dunham, I. et.al. | Plus | 33573387-33573517 |
| 339367 | Dunham, I. et.al. | Plus | 33575210-33575416 |
| 339360 | Dunham, I. et.al. | Plus | 33579433-33579593 |
| 339384 | Dunham, I. et.al. | Plus | 33950591-33950697 |
| 339413 | Dunham, I. et.al. | Plus | 34268734-34268875 |
| 339419 | Dunham, I. et.al. | Plus | 34359680-34359777 |
| 332854 | Dunham, I. et.al. | Minus | 1283811-1283053 |
| 332859 | Dunham, I. et.al. | Minus | 1358943-1358861 |
| 332896 | Dunham, I. et.al. | Minus | 1631841-1631422 |
| 332995 | Dunham, I. et.al. | Minus | 2708847-2708685 |
| 333014 | Dunham, I. et.al. | Minus | 2772479-2772387 |
| 333043 | Dunham, I. et.al. | Minus | 2920906-2920781 |
| 333054 | Dunham, I. et.al. | Minus | 2995314-2995219 |
| 333069 | Dunham, I. et.al. | Minus | 3040771-3040661 |
| 333074 | Dunham, I. et.al. | Minus | 3054171-3054008 |
| 333101 | Dunham, I. et.al. | Minus | 3208966-3208908 |
| 333140 | Dunham, I. et.al. | Minus | 3377220-3376309 |
| 333142 | Dunham, I. et.al. | Minus | 3474264-3474145 |
| 333175 | Dunham, I. et.al. | Minus | 3768113-3768051 |
| 333216 | Dunham, I. et.al. | Minus | 3967627-3967512 |
| 333219 | Dunham, I. et.al. | Minus | 3968531-3968423 |
| 333220 | Dunham, I. et.al. | Minus | 3969363-3968789 |
| 333240 | Dunham, I. et.al. | Minus | 4100495-4100281 |
| 333242 | Dunham, I. et.al. | Minus | 4104544-4104259 |
| 333251 | Dunham, I. et.al. | Minus | 4168970-4168870 |
| 333252 | Dunham, I. et.al. | Minus | 4172550-4172390 |
| 333257 | Dunham, I. et.al. | Minus | 4300724-4300578 |
| 333258 | Dunham, I. et.al. | Minus | 4301348-4301269 |
| 333271 | Dunham, I. et.al. | Minus | 4377786-4377589 |
| 333294 | Dunham, I. et.al. | Minus | 4538728-4538642 |
| 333296 | Dunham, I. et.al. | Minus | 4550766-4550644 |
| 333312 | Dunham, I. et.al. | Minus | 4638794-4638635 |
| 333313 | Dunham, I. et.al. | Minus | 4639397-4639277 |
| 333323 | Dunham, I. et.al. | Minus | 4655333-4655166 |
| 333329 | Dunham, I. et.al. | Minus | 4661779-4661672 |
| 333339 | Dunham, I. et.al. | Minus | 4679691-4679610 |
| 333346 | Dunham, I. et.al. | Minus | 4704564-4704466 |
| 333355 | Dunham, I. et.al. | Minus | 4727555-4727460 |
| 333408 | Dunham, I. et.al. | Minus | 4936879-4936661 |
| 333423 | Dunham, I. et.al. | Minus | 4958233-4957593 |
| 333424 | Dunham, I. et.al. | Minus | 4959694-4958713 |
| 333441 | Dunham, I. et.al. | Minus | 2708847-2708685 |
| 333466 | Dunham, I. et.al. | Minus | 2708847-2708685 |
| 333495 | Dunham, I. et.al. | Minus | 5404039-5403881 |
| 333496 | Dunham, I. et.al. | Minus | 5404643-5404523 |
| 333541 | Dunham, I. et.al. | Minus | 5859241-5859098 |
| 333545 | Dunham, I. et.al. | Minus | 5885539-5885064 |
| 333561 | Dunham, I. et.al. | Minus | 5903659-5903590 |
| 333568 | Dunham, I. et.al. | Minus | 5965072-5964999 |
| 333581 | Dunham, I. et.al. | Minus | 6151638-6151159 |
| 333582 | Dunham, I. et.al. | Minus | 6158522-6158322 |
| 333665 | Dunham, I. et.al. | Minus | 6975471-6975215 |
| 333713 | Dunham, I. et.al. | Minus | 7289490-7289337 |
| 333730 | Dunham, I. et.al. | Minus | 7489326-7489007 |
| 333737 | Dunham, I. et.al. | Minus | 7551981-7551842 |
| 333762 | Dunham, I. et.al. | Minus | 7679486-7679343 |
| 333815 | Dunham, I. et.al. | Minus | 7871914-7871764 |
| 333895 | Dunham, I. et.al. | Minus | 8193845-8193735 |
| 333903 | Dunham, I. et.al. | Minus | 8216956-4216810 |
| 333910 | Dunham, I. et.al. | Minus | 8230221-4230116 |
| 333944 | Dunham, I. et.al. | Minus | 8557051-8556936 |
| 333965 | Dunham, I. et.al. | Minus | 8627484-8626258 |
| 334035 | Dunham, I. et.al. | Minus | 9323508-9323292 |
| 334125 | Dunham, I. et.al. | Minus | 10433521-10432612 |
| 334137 | Dunham, I. et.al. | Minus | 10461063-10480708 |
| 334172 | Dunham, I. et.al. | Minus | 11644142-11644008 |
| 334175 | Dunham, I. et.al. | Minus | 11668659-11668597 |
| 334191 | Dunham, I. et.al. | Minus | 11932215-11932091 |
| 334196 | Dunham, I. et.al. | Minus | 11966322-11966182 |
| 334198 | Dunham, I. et.al. | Minus | 12032364-12032146 |
| 334215 | Dunham, I. et.al. | Minus | 12670395-12670077 |
| 334237 | Dunham, I. et.al. | Minus | 12994394-12993954 |
| 334254 | Dunham, I. et.al. | Minus | 13199268-13198999 |
| 334304 | Dunham, I. et.al. | Minus | 13455315-13455219 |
| 334358 | Dunham, I. et.al. | Minus | 13724372-13724201 |
| 334396 | Dunham, I. et.al. | Minus | 14164482-14164340 |
| 334399 | Dunham, I. et.al. | Minus | 14186289-14186163 |
| 334418 | Dunham, I. et.al. | Minus | 14237235-14237028 |
| 334470 | Dunham, I. et.al. | Minus | 14389581-14389442 |
| 334474 | Dunham, I. et.al. | Minus | 14391920-14391809 |
| 334476 | Dunham, I. et.al. | Minus | 14394777-14394589 |
| 334496 | Dunham, I. et.al. | Minus | 14480743-14480669 |
| 334540 | Dunham, I. et.al. | Minus | 14832365-14832224 |
| 334548 | Dunham, I. et.al. | Minus | 14837304-14837150 |
| 334560 | Dunham, I. et.al. | Minus | 14980648-14980485 |
| 334591 | Dunham, I. et.al. | Minus | 15035270-15035057 |
| 334593 | Dunham, I. et.al. | Minus | 15036374-15036242 |
| 334625 | Dunham, I. et.al. | Minus | 15244936-15243666 |
| 334650 | Dunham, I. et.al. | Minus | 15371251-15371178 |
| 334682 | Dunham, I. et.al. | Minus | 15520806-15520668 |
| 334690 | Dunham, I. et.al. | Minus | 15603055-15602918 |
| 334691 | Dunham, I. et.al. | Minus | 15615920-15611418 |
| 334745 | Dunham, I. et.al. | Minus | 16049960-16049653 |
| 334752 | Dunham, I. et.al. | Minus | 16118059-16117848 |
| 334759 | Dunham, I. et.al. | Minus | 16134427-16134207 |
| 334760 | Dunham, I. et.al. | Minus | 16136658-16136578 |
| 334764 | Dunham, I. et.al. | Minus | 16151208-16151104 |
| 334766 | Dunham, I. et.al. | Minus | 16154663-16154529 |
| 334779 | Dunham, I. et.al. | Minus | 16276881-16276815 |
| 334782 | Dunham, I. et.al. | Minus | 16291693-16291574 |
| 334783 | Dunham, I. et.al. | Minus | 16293336-16293226 |
| 334784 | Dunham, I. et.al. | Minus | 16294548-16294360 |
| 334785 | Dunham, I. et.al. | Minus | 16295583-16295408 |
| 334788 | Dunham, I. et.al. | Minus | 16301237-16301081 |
| 334793 | Dunham, I. et.al. | Minus | 16330748-16330681 |
| 334804 | Dunham, I. et.al. | Minus | 16428342-16428208 |
| 334823 | Dunham, I. et.al. | Minus | 16851360-16851189 |
| 334827 | Dunham, I. et.al. | Minus | 16905877-16905684 |
| 334836 | Dunham, I. et.al. | Minus | 17215581-17215418 |
| 334842 | Dunham, I. et.al. | Minus | 17464352-17464181 |
| 334946 | Dunham, I. et.al. | Minus | 20140514-20140410 |
| 334948 | Dunham, I. et.al. | Minus | 20141727-20141583 |
| 334955 | Dunham, I. et.al. | Minus | 20151753-20151684 |
| 334958 | Dunham, I. et.al. | Minus | 20156046-20155932 |
| 334966 | Dunham, I. et.al. | Minus | 20169171-20169079 |
| 334969 | Dunham, I. et.al. | Minus | 20188176-20188020 |
| 334970 | Dunham, I. et.al. | Minus | 20195886-20195554 |
| 335000 | Dunham, I. et.al. | Minus | 20455403-20455348 |
| 335002 | Dunham, I. et.al. | Minus | 20479143-20479008 |
| 335009 | Dunham, I. et.al. | Minus | 20602011-20601951 |
| 335068 | Dunham, I. et.al. | Minus | 20955606-20955458 |
| 335090 | Dunham, I. et.al. | Minus | 21192979-21190943 |
| 335092 | Dunham, I. et.al. | Minus | 21298638-21298571 |
| 335108 | Dunham, I. et.al. | Minus | 21324221-21324030 |
| 335115 | Dunham, I. et.al. | Minus | 21388250-21388146 |
| 335116 | Dunham, I. et.al. | Minus | 21388573-21388414 |
| 335217 | Dunham, I. et.al. | Minus | 21801622-21801195 |
| 335234 | Dunham, I. et.al. | Minus | 21904287-21904080 |
| 335236 | Dunham, I. et.al. | Minus | 21915016-21914870 |
| 335247 | Dunham, I. et.al. | Minus | 21944287-21944209 |
| 335262 | Dunham, I. et.al. | Minus | 22065821-22065630 |
| 335265 | Dunham, I. et.al. | Minus | 22102127-22102000 |
| 335266 | Dunham, I. et.al. | Minus | 22108492-22108411 |
| 335281 | Dunham, I. et.al. | Minus | 22237614-22237503 |
| 335331 | Dunham, I. et.al. | Minus | 22556823-22556708 |
| 335339 | Dunham, I. et.al. | Minus | 22569208-22569045 |
| 335340 | Dunham, I. et.al. | Minus | 22570500-22570342 |
| 335344 | Dunham, I. et.al. | Minus | 22600566-22600498 |
| 335348 | Dunham, I. et.al. | Minus | 22646377-22646122 |
| 335349 | Dunham, I. et.al. | Minus | 22661861-22661271 |
| 335523 | Dunham, I. et.al. | Minus | 24235282-24235175 |
| 335527 | Dunham, I. et.al. | Minus | 24241519-24241370 |
| 335566 | Dunham, I. et.al. | Minus | 24892522-24891998 |
| 335606 | Dunham, I. et.al. | Minus | 25056719-25055010 |
| 335750 | Dunham, I. et.al. | Minus | 25732501-25731972 |
| 335755 | Dunham, I. et.al. | Minus | 25763806-25763747 |
| 335768 | Dunham, I. et.al. | Minus | 25819821-25819690 |
| 335774 | Dunham, I. et.al. | Minus | 25883733-25883572 |
| 335777 | Dunham, I. et.al. | Minus | 25885770-25885599 |
| 335782 | Dunham, I. et.al. | Minus | 25908578-25908440 |
| 335787 | Dunham, I. et.al. | Minus | 25947022-25946835 |
| 335806 | Dunham, I. et.al. | Minus | 26217475-26217146 |
| 335817 | Dunham, I. et.al. | Minus | 26321875-26321750 |
| 335827 | Dunham, I. et.al. | Minus | 26380557-26380472 |
| 335831 | Dunham, I. et.al. | Minus | 26321526-26321349 |
| 335832 | Dunham, I. et.al. | Minus | 26383701-26383583 |
| 335863 | Dunham, I. et.al. | Minus | 26693017-26692763 |
| 335895 | Dunham, I. et.al. | Minus | 26975307-26975239 |
| 335902 | Dunham, I. et.al. | Minus | 26985491-26985358 |
| 335920 | Dunham, I. et.al. | Minus | 27034927-27034811 |
| 335956 | Dunham, I. et.al. | Minus | 27653729-27653635 |
| 335980 | Dunham, I. et.al. | Minus | 27846751-27846614 |
| 335999 | Dunham, I. et.al. | Minus | 28033986-28033848 |
| 336000 | Dunham, I. et.al. | Minus | 28105547-28105354 |
| 336029 | Dunham, I. et.al. | Minus | 28732042-28731966 |
| 336142 | Dunham, I. et.al. | Minus | 30135157-30135054 |
| 336149 | Dunham, I. et.al. | Minus | 30148185-30148059 |
| 336161 | Dunham, I. et.al. | Minus | 30169370-30169185 |
| 336177 | Dunham, I. et.al. | Minus | 30306638-30303284 |
| 336198 | Dunham, I. et.al. | Minus | 30459668-30459460 |
| 336199 | Dunham, I. et.al. | Minus | 30461245-30461040 |
| 336200 | Dunham, I. et.al. | Minus | 30462971-30462895 |
| 336202 | Dunham, I. et.al. | Minus | 30468933-30466781 |
| 336203 | Dunham, I. et.al. | Minus | 30470796-30470653 |
| 336227 | Dunham, I. et.al. | Minus | 30902014-30901946 |
| 336231 | Dunham, I. et.al. | Minus | 30967656-30967471 |
| 336232 | Dunham, I. et.al. | Minus | 30977707-30977645 |
| 336238 | Dunham, I. et.al. | Minus | 31160083-31159959 |
| 336243 | Dunham, I. et.al. | Minus | 31402237-31402104 |
| 336246 | Dunham, I. et.al. | Minus | 31425669-31425253 |
| 336279 | Dunham, I. et.al. | Minus | 32103203-32102860 |
| 336280 | Dunham, I. et.al. | Minus | 32103588-32103497 |
| 336294 | Dunham, I. et.al. | Minus | 32946997-32946890 |
| 336295 | Dunham, I. et.al. | Minus | 32950291-32950162 |
| 336328 | Dunham, I. et.al. | Minus | 33584461-33584201 |
| 336347 | Dunham, I. et.al. | Minus | 33843218-33843104 |
| 336351 | Dunham, I. et.al. | Minus | 33865890-33865681 |
| 336397 | Dunham, I. et.al. | Minus | 34021504-34021389 |
| 336400 | Dunham, I. et.al. | Minus | 34023437-34023298 |
| 336402 | Dunham, I. et.al. | Minus | 34024090-34023981 |
| 336406 | Dunham, I. et.al. | Minus | 34030774-34030564 |
| 336417 | Dunham, I. et.al. | Minus | 34047771-34047675 |
| 336455 | Dunham, I. et.al. | Minus | 34209155-34209018 |
| 336463 | Dunham, I. et.al. | Minus | 34212236-34211968 |
| 336479 | Dunham, I. et.al. | Minus | 34218224-34218139 |
| 336510 | Dunham, I. et.al. | Minus | 34277046-34276928 |
| 336538 | Dunham, I. et.al. | Minus | 34329270-34329189 |
| 336584 | Dunham, I. et.al. | Minus | 34516568-34516468 |
| 336599 | Dunham, I. et.al. | Minus | 11736500-11734418 |
| 336615 | Dunham, I. et.al. | Minus | 26020622-26016546 |
| 336616 | Dunham, I. et.al. | Minus | 26021027-26020848 |
| 336618 | Dunham, I. et.al. | Minus | 25799-25612 |
| 336645 | Dunham, I. et.al. | Minus | 1351268-1351168 |
| 336653 | Dunham, I. et.al. | Minus | 1568327-1568216 |
| 336657 | Dunham, I. et.al. | Minus | 1705497-1705415 |
| 336717 | Dunham, I. et.al. | Minus | 3297417-3297352 |
| 336772 | Dunham, I. et.al. | Minus | 5129939-5129785 |
| 336795 | Dunham, I. et.al. | Minus | 5681580-5681460 |
| 336796 | Dunham, I. et.al. | Minus | 5683253-5683016 |
| 336854 | Dunham, I. et.al. | Minus | 7884839-7884767 |
| 336858 | Dunham, I. et.al. | Minus | 8200946-8200789 |
| 336882 | Dunham, I. et.al. | Minus | 8394593-8394369 |
| 336863 | Dunham, I. et.al. | Minus | 8396673-8396425 |
| 336883 | Dunham, I. et.al. | Minus | 9322767-9322554 |
| 336908 | Dunham, I. et.al. | Minus | 10791347-10791256 |
| 336927 | Dunham, I. et.al. | Minus | 11548111-11547937 |
| 336929 | Dunham, I. et.al. | Minus | 11603040-11602908 |
| 336978 | Dunham, I. et.al. | Minus | 14241223-14241030 |
| 336981 | Dunham, I. et.al. | Minus | 14478638-14478472 |
| 336984 | Dunham, I. et.al. | Minus | 14725450-14725309 |
| 336994 | Dunham, I. et.al. | Minus | 15104752-15104656 |
| 336999 | Dunham, I. et.al. | Minus | 15389579-15388897 |
| 337011 | Dunham, I. et.al. | Minus | 16106423-16106080 |
| 337023 | Dunham, I. et.al. | Minus | 16346174-16346065 |
| 337069 | Dunham, I. et.al. | Minus | 19014379-19014222 |
| 337092 | Dunham, I. et.al. | Minus | 20140132-20139980 |
| 337093 | Dunham, I. et.al. | Minus | 20145693-20145570 |
| 337094 | Dunham, I. et.al. | Minus | 20146915-20146778 |
| 337097 | Dunham, I. et.al. | Minus | 20498648-20497686 |
| 337100 | Dunham, I. et.al. | Minus | 20543630-20543751 |
| 337114 | Dunham, I. et.al. | Minus | 21328259-21328050 |
| 337203 | Dunham, I. et.al. | Minus | 25349286-25349156 |
| 337204 | Dunham, I. et.al. | Minus | 25419024-25418931 |
| 337225 | Dunham, I. et.al. | Minus | 26607638-26607468 |
| 337244 | Dunham, I. et.al. | Minus | 27362048-27361952 |
| 337279 | Dunham, I. et.al. | Minus | 28430185-28430033 |
| 337289 | Dunham, I. et.al. | Minus | 28756246-28756163 |
| 337316 | Dunham, I. et.al. | Minus | 29657129-29656997 |
| 337431 | Dunham, I. et.al. | Minus | 32110529-32110475 |
| 337432 | Dunham, I. et.al. | Minus | 32199134-32198922 |
| 337445 | Dunham, I. et.al. | Minus | 32292380-32292228 |
| 337451 | Dunham, I. et.al. | Minus | 32408680-32408576 |
| 337452 | Dunham, I. et.al. | Minus | 32415187-32415117 |
| 337455 | Dunham, I. et.al. | Minus | 32434517-32434425 |
| 337456 | Dunham, I. et.al. | Minus | 32438887-32438828 |
| 337513 | Dunham, I. et.al. | Minus | 33478692-33478562 |
| 337514 | Dunham, I. et.al. | Minus | 33484408-33484339 |
| 337517 | Dunham, I. et.al. | Minus | 33795568-33795385 |
| 337590 | Dunham, I. et.al. | Minus | 982397-982246 |
| 337592 | Dunham, I. et.al. | Minus | 1007791-1007634 |
| 337604 | Dunham, I. et.al. | Minus | 1329838-1329759 |
| 337606 | Dunham, I. et.al. | Minus | 1346786-1346617 |
| 337612 | Dunham, I. et.al. | Minus | 1570235-1570142 |
| 337645 | Dunham, I. et.al. | Minus | 5141462-5141329 |
| 337704 | Dunham, I. et.al. | Minus | 3658670-3658545 |
| 337706 | Dunham, I. et.al. | Minus | 3672056-3671922 |
| 337760 | Dunham, I. et.al. | Minus | 4008389-4008037 |
| 337768 | Dunham, I. et.al. | Minus | 4050134-4049973 |
| 337771 | Dunham, I. et.al. | Minus | 4060797-4060707 |
| 337778 | Dunham, I. et.al. | Minus | 4087301-4087157 |
| 337787 | Dunham, I. et.al. | Minus | 4135097-4135025 |
| 337816 | Dunham, I. et.al. | Minus | 4523203-4523090 |
| 337821 | Dunham, I. et.al. | Minus | 4538189-4538075 |
| 337848 | Dunham, I. et.al. | Minus | 5053964-5053829 |
| 337911 | Dunham, I. et.al. | Minus | 5866504-5866154 |
| 337913 | Dunham, I. et.al. | Minus | 6149843-6149786 |
| 337974 | Dunham, I. et.al. | Minus | 7153401-7153085 |
| 337984 | Dunham, I. et.al. | Minus | 7282160-7282020 |
| 338069 | Dunham, I. et.al. | Minus | 8728655-8728580 |
| 338087 | Dunham, I. et.al. | Minus | 9362067-9361878 |
| 338113 | Dunham, I. et.al. | Minus | 10481242-10481089 |
| 338132 | Dunham, I. et.al. | Minus | 10989617-10989530 |
| 338140 | Dunham, I. et.al. | Minus | 11237977-11237876 |
| 338151 | Dunham, I. et.al. | Minus | 11519629-11519501 |
| 338221 | Dunham, I. et.al. | Minus | 14183649-14183568 |
| 338223 | Dunham, I. et.al. | Minus | 14287001-14286830 |
| 338235 | Dunham, I. et.al. | Minus | 14484254-14484179 |
| 338271 | Dunham, I. et.al. | Minus | 16048151-16047985 |
| 338285 | Dunham, I. et.al. | Minus | 16276030-16274895 |
| 338306 | Dunham, I. et.al. | Minus | 16966519-16966247 |
| 338325 | Dunham, I. et.al. | Minus | 17241596-17241507 |
| 338368 | Dunham, I. et.al. | Minus | 18272087-18272025 |
| 338381 | Dunham, I. et.al. | Minus | 18534404-18534234 |
| 338431 | Dunham, I. et.al. | Minus | 19747608-19747496 |
| 338450 | Dunham, I. et.al. | Minus | 20172384-20172200 |
| 338451 | Dunham, I. et.al. | Minus | 20174286-20174193 |
| 338486 | Dunham, I. et.al. | Minus | 21063152-21063049 |
| 338508 | Dunham, I. et.al. | Minus | 21308161-21307923 |
| 338576 | Dunham, I. et.al. | Minus | 22676619-22676368 |
| 338591 | Dunham, I. et.al. | Minus | 22959271-22959182 |
| 338593 | Dunham, I. et.al. | Minus | 22992718-22992531 |
| 338594 | Dunham, I. et.al. | Minus | 23026144-23026029 |
| 338668 | Dunham, I. et.al. | Minus | 24500606-24500442 |
| 338686 | Dunham, I. et.al. | Minus | 24836463-24838246 |
| 338725 | Dunham, I. et.al. | Minus | 25904148-25904017 |
| 338819 | Dunham, I. et.al. | Minus | 27539222-27539058 |
| 338829 | Dunham, I. et.al. | Minus | 27739328-27739163 |
| 338843 | Dunham, I. et.al. | Minus | 27899321-27899045 |
| 338934 | Dunham, I. et.al. | Minus | 29036147-29036048 |
| 338976 | Dunham, I. et.al. | Minus | 29867484-29867259 |
| 338990 | Dunham, I. et.al. | Minus | 30019967-30019837 |
| 339019 | Dunham, I. et.al. | Minus | 30498278-30498118 |
| 339032 | Dunham, I. et.al. | Minus | 30613407-30613275 |
| 339033 | Dunham, I. et.al. | Minus | 30621102-30620830 |
| 339037 | Dunham, I. et.al. | Minus | 30634104-30633988 |
| 339044 | Dunham, I. et.al. | Minus | 30721853-30721740 |
| 339071 | Dunham, I. et.al. | Minus | 30885702-30885511 |
| 339127 | Dunham, I. et.al. | Minus | 31688232-31688072 |
| 339130 | Dunham, I. et.al. | Minus | 31743961-31743767 |
| 339181 | Dunham, I. et.al. | Minus | 32321697-32321571 |
| 339188 | Dunham, I. et.al. | Minus | 32347554-32347250 |
| 339193 | Dunham, I. et.al. | Minus | 32415060-32414928 |
| 339196 | Dunham, I. et.al. | Minus | 32431422-32431097 |
| 339208 | Dunham, I. et.al. | Minus | 32491714-32491657 |
| 339213 | Dunham, I. et.al. | Minus | 32496590-32496440 |
| 339215 | Dunham, I. et.al. | Minus | 32502559-32502383 |
| 339220 | Dunham, I. et.al. | Minus | 32519184-32519081 |
| 339230 | Dunham, I. et.al. | Minus | 32729004-32728929 |
| 339251 | Dunham, I. et.al. | Minus | 32894314-32894216 |
| 339256 | Dunham, I. et.al. | Minus | 32926055-32925967 |
| 339266 | Dunham, I. et.al. | Minus | 32976148-32976041 |
| 339288 | Dunham, I. et.al. | Minus | 32984700-32984500 |
| 339280 | Dunham, I. et.al. | Minus | 33114230-33114010 |
| 339340 | Dunham, I. et.al. | Minus | 33485220-33485144 |
| 339342 | Dunham, I. et.al. | Minus | 33494677-33494541 |
| 339344 | Dunham, I. et.al. | Minus | 33503675-33503520 |
| 339363 | Dunham, I. et.al. | Minus | 33589964-33589665 |
| 339365 | Dunham, I. et.al. | Minus | 33641615-33641446 |
| 339401 | Dunham, I. et.al. | Minus | 34045393-34045138 |
| 339424 | Dunham, I. et.al. | Minus | 34392464-34392311 |
| 339435 | Dunham, I. et.al. | Minus | 34538987-34538876 |
| 337884 | | | |
| 337885 | | | |
| 337895 | | | |
| 337898 | | | |
| 329611 | 3962478 | Minus | 11780-11875 |
| 329496 | 3983518 | Plus | 13570-13686 |
| 325310 | 5866864 | Minus | 17256-17357 |
| 325320 | 5866670 | Minus | 33200-33646 |
| 325338 | 5666883 | Minus | 62747-62928 |
| 325271 | 5866901 | Plus | 87318-87378 |
| 325285 | 5866903 | Plus | 107772-107864 |
| 325289 | 5866903 | Minus | 129715-129877 |
| 325300 | 5866908 | Minus | 7940-8059 |
| 325304 | 5866910 | Minus | 37466-37884 |
| 329630 | 6729060 | Minus | 167326-167511 |
| 325369 | 5866920 | Minus | 1018736-1018830 |
| 325359 | 5866920 | Plus | 392668-392781 |
| 325408 | 5866921 | Plus | 599118-599289 |
| 325409 | 5866921 | Plus | 599438-599655 |
| 325410 | 5866921 | Plus | 615477-615580 |
| 325393 | 5866921 | Minus | 350513-350691 |
| 325396 | 5866921 | Plus | 476868-477200 |
| 325433 | 5866936 | Minus | 480706-480826 |
| 325448 | 5866941 | Minus | 392834-392982 |
| 325464 | 5866947 | Plus | 323995-324279 |
| 325480 | 5866957 | Plus | 20558-21112 |
| 325482 | 5866957 | Plus | 47957-48078 |
| 325510 | 5866974 | Plus | 219958-220917 |
| 325524 | 5866981 | Minus | 117837-117997 |
| 325513 | 6017035 | Minus | 34295-34490 |
| 325519 | 6017036 | Minus | 186804-186915 |
| 325571 | 6552439 | Minus | 184952-185135 |
| 329638 | 3779004 | Minus | 4636-5127 |
| 325623 | 5867000 | Plus | 84366-84472 |
| 325636 | 5867002 | Minus | 149928-150033 |
| 325677 | 5867017 | Minus | 58414-58518 |
| 325691 | 5867021 | Plus | 65467-65629 |
| 325690 | 5867021 | Plus | 7391-7530 |
| 325702 | 5867028 | Plus | 139925-140061 |
| 325703 | 5867028 | Plus | 155806-156098 |
| 325790 | 6381957 | Minus | 105385-105524 |
| 325783 | 6456780 | Plus | 102815-102957 |
| 325695 | 6552446 | Minus | 199599-199778 |
| 325726 | 6552447 | Plus | 154262-154623 |
| 325742 | 6552448 | Minus | 38225-38306 |
| 325760 | 6552449 | Plus | 343394-343549 |
| 325801 | 6552451 | Plus | 49269-49542 |
| 325803 | 6552451 | Plus | 53355-53558 |
| 325649 | 6588011 | Minus | 215659-215815 |
| 325710 | 6682473 | Plus | 204538-204822 |
| 325712 | 6682473 | Plus | 208541-208926 |
| 325755 | 6682474 | Plus | 406603-407037 |
| 325751 | 6682474 | Plus | 130437-130520 |
| 325763 | 6682475 | Minus | 327530-327910 |
| 325791 | 6682476 | Plus | 344059-344841 |
| 329782 | 5912597 | Plus | 123410-123499 |
| 329779 | 6002090 | Minus | 195353-195669 |
| 329760 | 6048280 | Minus | 51419-51528 |
| 329752 | 6065777 | Plus | 132916-133335 |
| 329737 | 6065779 | Plus | 70136-70236 |
| 329735 | 6065780 | Plus | 63923-64213 |
| 329719 | 6065785 | Plus | 46970-47762 |
| 329725 | 6065785 | Plus | 133747-133842 |
| 329705 | 6065790 | Minus | 63183-63724 |
| 329665 | 6272129 | Plus | 91791-91891 |
| 329793 | 6522661 | Minus | 96556-96698 |
| 329824 | 6630758 | Minus | 126072-126254 |
| 329839 | 6672062 | Plus | 34377-34537 |
| 329853 | 6682295 | Plus | 68945-69009 |
| 329870 | 6706435 | Minus | 5729-5870 |
| 329879 | 6466518 | Minus | 12266-12356 |
| 329902 | 6634760 | Plus | 106906-106984 |
| 325851 | 5867067 | Minus | 51751-51981 |
| 325886 | 5867087 | Plus | 194694-194915 |
| 325887 | 5867087 | Plus | 195052-195485 |
| 326005 | 5867112 | Minus | 7827-8117 |
| 325945 | 5867138 | Minus | 123079-124086 |
| 325953 | 5867140 | Minus | 4721-4901 |
| 325965 | 5867147 | Plus | 216364-216516 |
| 325966 | 5867147 | Plus | 217235-217356 |
| 325977 | 6249602 | Plus | 60638-60759 |
| 325835 | 6552452 | Minus | 81182-81414 |
| 329995 | 4567166 | Minus | 150014-150315 |
| 329948 | 5540101 | Minus | 134056-134261 |
| 329940 | 6165199 | Minus | 33718-33882 |
| 329921 | 6165205 | Minus | 33759-33891 |
| 330002 | 6623963 | Plus | 46097-46158 |
| 330021 | 6671889 | Plus | 120938-121032 |
| 326162 | 5867168 | Minus | 5870-6291 |
| 326070 | 5867175 | Plus | 132181-132731 |
| 326029 | 5867176 | Minus | 112558-112669 |
| 326033 | 5867178 | Plus | 37261-37333 |
| 326039 | 5867179 | Minus | 15157-15227 |
| 326122 | 5867194 | Plus | 144397-144683 |
| 326136 | 5867202 | Minus | 155973-156065 |
| 326194 | 5867213 | Plus | 75271-75522 |
| 326206 | 5867219 | Plus | 113881-115080 |
| 326218 | 5867226 | Minus | 259784-259920 |
| 326224 | 5867230 | Plus | 21528-21667 |
| 326240 | 5867260 | Plus | 94843-96127 |
| 326253 | 5867263 | Minus | 222216-222341 |
| 326249 | 5867263 | Minus | 80253-80323 |
| 326266 | 5867264 | Plus | 337993-338192 |
| 326304 | 5867277 | Minus | 367450-367559 |
| 326309 | 5867277 | Plus | 614150-614294 |
| 326310 | 5867277 | Plus | 621555-621698 |
| 326338 | 6056311 | Minus | 161972-162333 |
| 326343 | 6525295 | Minus | 2699-2777 |
| 326344 | 6525295 | Minus | 3207-3365 |
| 326073 | 6682495 | Minus | 248865-249009 |
| 330057 | 6478962 | Plus | 75145-75287 |
| 330058 | 6634847 | Plus | 100602-100963 |
| 330061 | 6721261 | Plus | 4254-4388 |
| 326539 | 5867307 | Plus | 198357-198504 |
| 326545 | 5867307 | Plus | 600850-601425 |
| 326549 | 5867307 | Plus | 698004-698069 |
| 326552 | 5867308 | Minus | 6988-7107 |
| 326554 | 5867308 | Plus | 16259-18271 |
| 326559 | 5867310 | Plus | 159233-159439 |
| 326577 | 5867317 | Plus | 215987-216127 |
| 326380 | 5867327 | Plus | 32474-32668 |
| 326412 | 5867362 | Minus | 9263-9436 |
| 326417 | 5867362 | Plus | 89132-89383 |
| 326418 | 5867365 | Plus | 23609-23745 |
| 326423 | 5867369 | Minus | 142311-142500 |
| 326458 | 5867400 | Plus | 128045-128147 |
| 326459 | 5867400 | Plus | 137590-138212 |
| 326506 | 5867435 | Minus | 9368-9509 |
| 326509 | 6682496 | Plus | 92406-92680 |
| 330112 | 6015238 | Plus | 32059-32163 |
| 330086 | 6015293 | Plus | 29518-29662 |
| 330080 | 6015314 | Minus | 4890-4972 |
| 330082 | 6015314 | Plus | 27158-27296 |
| 330064 | 6165044 | Minus | 5619-5696 |
| 330063 | 6165044 | Minus | 948-1025 |
| 330065 | 6165044 | Minus | 9876-9948 |
| 326646 | 5867562 | Plus | 100426-100608 |
| 326688 | 5867582 | Plus | 104875-105531 |
| 326708 | 5867593 | Minus | 39203-39310 |
| 326710 | 5867593 | Minus | 80204-80468 |
| 326746 | 5867611 | Plus | 129366-129565 |
| 326793 | 5867631 | Plus | 136295-136518 |
| 326603 | 6056312 | Minus | 50323-50575 |
| 326806 | 6469835 | Plus | 101628-102149 |
| 326783 | 6525298 | Plus | 196378-196863 |
| 326668 | 6552455 | Plus | 146726-146838 |
| 326725 | 6552456 | Minus | 223005-223125 |
| 326857 | 6552460 | Minus | 120142-120345 |
| 326763 | 6598307 | Plus | 239690-239902 |
| 326808 | 6682504 | Minus | 65107-65214 |
| 326874 | 6682507 | Minus | 4643-4888 |
| 326876 | 6682507 | Minus | 46203-46499 |
| 326884 | 6682511 | Plus | 33403-33479 |
| 326997 | 5867660 | Minus | 71389-72147 |
| 327009 | 5867654 | Plus | 933145-933266 |
| 327015 | 5867664 | Plus | 1030457-1030534 |
| 326942 | 6004446 | Minus | 88675-88785 |
| 326943 | 6004446 | Minus | 89242-89427 |
| 326957 | 6469836 | Plus | 37529-37694 |
| 327049 | 6531965 | Minus | 1924026-1924110 |
| 327051 | 6531965 | Plus | 2222592-2222717 |
| 327056 | 6531965 | Plus | 2374323-2374751 |
| 327059 | 6531965 | Plus | 2421032-2421799 |
| 327067 | 6531965 | Minus | 3726023-3726668 |
| 327089 | 6531965 | Plus | 5238983-5239187 |
| 327037 | 6531965 | Minus | 387854-388045 |
| 327123 | 6531971 | Minus | 23980-24340 |
| 327125 | 6531971 | Plus | 48884-48975 |
| 326981 | 6588016 | Plus | 105091-106038 |
| 330139 | 4210430 | Plus | 112089-112450 |
| 330153 | 4325335 | Plus | 146951-147475 |
| 330130 | 6002196 | Minus | 241903-241967 |
| 327203 | 5867447 | Plus | 36485-36577 |
| 327206 | 5867447 | Plus | 177855-178031 |
| 327259 | 5867454 | Plus | 87268-87438 |
| 327264 | 5867461 | Plus | 47014-47367 |
| 327273 | 5867466 | Plus | 73451-73549 |
| 327274 | 5867470 | Minus | 84027-84128 |
| 327277 | 5867473 | Minus | 165616-165715 |
| 327278 | 5867473 | Minus | 166350-166439 |
| 327289 | 5867481 | Plus | 49296-49536 |
| 327304 | 5867494 | Plus | 20664-20850 |
| 327315 | 5867508 | Minus | 78409-79245 |
| 327246 | 5867547 | Plus | 136212-136325 |
| 327155 | 5867549 | Plus | 90343-90876 |
| 327159 | 5867550 | Minus | 8219-8331 |
| 327334 | 5902477 | Minus | 142655-142745 |
| 327341 | 6017016 | Minus | 122906-123014 |
| 327185 | 6117805 | Minus | 3287-3451 |
| 327309 | 6456757 | Minus | 10219-10457 |
| 327263 | 6525274 | Minus | 153814-154920 |
| 327362 | 6552412 | Minus | 62459-62805 |
| 327413 | 5867750 | Plus | 101410-101508 |
| 327418 | 5867750 | Minus | 153453-153547 |
| 327430 | 5867754 | Plus | 1320-1403 |
| 327431 | 5867754 | Plus | 1853-1958 |
| 327472 | 5867775 | Plus | 74628-74937 |
| 327487 | 5867785 | Minus | 146220-146326 |
| 327379 | 5867795 | Plus | 1368-1820 |
| 327461 | 6004455 | Plus | 209031-209210 |
| 327532 | 6469818 | Plus | 71994-72137 |
| 330170 | 6648220 | Plus | 103280-103849 |
| 330166 | 6648220 | Plus | 86542-86867 |
| 327544 | 5867797 | Minus | 18105-18332 |
| 327564 | 5867811 | Plus | 13850-14018 |
| 327566 | 5867811 | Plus | 33383-33901 |
| 327581 | 5867825 | Plus | 5318-5434 |
| 327585 | 5867825 | Plus | 85660-85764 |
| 327605 | 6004463 | Plus | 199214-199579 |
| 327710 | 5867860 | Minus | 131012-131790 |
| 327610 | 5867868 | Minus | 174109-174278 |
| 327624 | 5867871 | Minus | 37699-37788 |
| 327641 | 5867890 | Plus | 13583-13702 |
| 327646 | 5867894 | Minus | 3043-3258 |
| 327614 | 6525283 | Plus | 3634-4001 |
| 327736 | 5867940 | Minus | 37781-37887 |
| 327739 | 5867942 | Minus | 182187-182548 |
| 327740 | 5867943 | Plus | 25716-26077 |
| 327743 | 5867944 | Minus | 155930-156098 |
| 327755 | 5867955 | Minus | 61969-62145 |
| 327772 | 5867964 | Minus | 26185-26285 |
| 327774 | 5867964 | Minus | 127659-127899 |
| 327823 | 5867968 | Minus | 170359-170433 |
| 327827 | 5867968 | Minus | 201918-202048 |
| 327833 | 5867968 | Minus | 303618-303732 |
| 327805 | 5867968 | Plus | 19952-20019 |
| 327809 | 5867968 | Plus | 54610-54761 |
| 327816 | 5867968 | Minus | 79202-79552 |
| 327790 | 5867977 | Plus | 19822-19985 |
| 327791 | 5867977 | Plus | 22491-22610 |
| 327793 | 5867979 | Plus | 18874-19254 |
| 327845 | 6531962 | Plus | 193402-193549 |
| 327846 | 6531962 | Plus | 195216-195373 |
| 330204 | 6013606 | Plus | 86663-86811 |
| 330189 | 6165182 | Minus | 26732-26991 |
| 330239 | 6671857 | Plus | 117484-118092 |
| 330266 | 6671885 | Minus | 129505-129832 |
| 330275 | 6671904 | Plus | 103585-103716 |
| 330280 | 6671910 | Plus | 2109-2377 |
| 330286 | 6671913 | Minus | 31050-31171 |
| 327999 | 5867994 | Plus | 94710-94841 |
| 328109 | 5868020 | Minus | 353895-354525 |
| 328098 | 5868020 | Minus | 261745-261920 |
| 328134 | 5868039 | Plus | 72354-72487 |
| 328171 | 5868071 | Plus | 101102-101224 |
| 328221 | 5868099 | Minus | 37489-37829 |
| 328224 | 5868101 | Plus | 105563-105832 |
| 328228 | 5868105 | Minus | 21488-21596 |
| 328236 | 5868117 | Plus | 13864-14371 |
| 327864 | 5868130 | Plus | 59139-59358 |
| 327888 | 5868149 | Minus | 51964-52120 |
| 327899 | 5868156 | Minus | 102288-102697 |
| 327925 | 5868172 | Minus | 118396-118490 |
| 327927 | 5868173 | Plus | 50989-51246 |
| 327937 | 5868192 | Minus | 33127-33485 |
| 327946 | 5868206 | Plus | 44102-44319 |
| 327982 | 5868216 | Plus | 30307-30527 |
| 327990 | 5868218 | Minus | 36225-36503 |
| 328015 | 5902482 | Minus | 477679-478113 |
| 328016 | 5902482 | Minus | 507572-508519 |
| 328025 | 5902482 | Minus | 931937-932171 |
| 328031 | 5902482 | Plus | 1176372-1177283 |
| 328053 | 5902482 | Minus | 2709850-2710010 |
| 328243 | 6056292 | Plus | 1-243 |
| 328271 | 6552415 | Plus | 39015-39098 |
| 328592 | 5868227 | Minus | 252407-252565 |
| 328570 | 5868231 | Plus | 89210-89816 |
| 328607 | 5868233 | Minus | 246798-246944 |
| 328620 | 5868241 | Minus | 15651-15788 |
| 328624 | 5868246 | Minus | 120666-120836 |
| 328791 | 5868309 | Plus | 171592-171929 |
| 328810 | 5868327 | Plus | 101730-101914 |
| 328820 | 5868330 | Plus | 90446-90602 |
| 328835 | 5868339 | Plus | 88053-88461 |
| 328282 | 5868353 | Plus | 72692-72819 |
| 328314 | 5868371 | Minus | 288397-288505 |
| 328328 | 5868375 | Plus | 169210-169407 |
| 328420 | 5868411 | Plus | 53612-53886 |
| 328428 | 5868417 | Plus | 13599-13780 |
| 328436 | 5868417 | Plus | 203760-203904 |
| 328444 | 5868420 | Plus | 65393-66103 |
| 328462 | 5868433 | Plus | 49649-49768 |
| 328467 | 5868434 | Minus | 15954-16073 |
| 328474 | 5868446 | Minus | 128777-128970 |
| 328484 | 5868454 | Minus | 21974-22140 |
| 328504 | 5868471 | Plus | 47064-47217 |
| 328506 | 5868471 | Plus | 60716-60830 |
| 328507 | 5868473 | Minus | 199637-199990 |
| 328544 | 5868486 | Plus | 145659-145829 |
| 328552 | 5868489 | Plus | 47328-47607 |
| 328557 | 5868489 | Plus | 138094-138161 |
| 328558 | 5868489 | Plus | 143648-144108 |
| 328276 | 6004471 | Plus | 13282-13450 |
| 328277 | 6004471 | Minus | 279901-280181 |
| 328662 | 6004473 | Plus | 1184773-1184855 |
| 328636 | 6004473 | Plus | 192484-192543 |
| 328803 | 6004475 | Minus | 291716-291948 |
| 328305 | 6004478 | Minus | 34730-34851 |
| 328569 | 6004480 | Plus | 232896-233243 |
| 328581 | 6006033 | Minus | 121249-121400 |
| 328582 | 6006033 | Minus | 134177-134282 |
| 328768 | 6017031 | Minus | 223741-224238 |
| 328770 | 6017031 | Minus | 363933-364166 |
| 328841 | 6381920 | Minus | 5214-5479 |
| 328851 | 6381923 | Plus | 2502-2606 |
| 328859 | 6381928 | Plus | 69045-69138 |
| 328860 | 6381928 | Plus | 83265-83366 |
| 328863 | 6381929 | Minus | 29313-29506 |
| 328868 | 6381930 | Plus | 112825-112993 |
| 328876 | 6525286 | Plus | 94053-94185 |
| 328886 | 6588003 | Plus | 31068-31429 |
| 328888 | 6588003 | Minus | 111901-111999 |
| 328936 | 5868500 | Minus | 1352202-1352259 |
| 328938 | 5868500 | Plus | 1522923-1522986 |
| 328971 | 6478806 | Minus | 23976-24105 |
| 330338 | 5457162 | Plus | 48406-48518 |
| 330327 | 5919194 | Plus | 121561-121683 |
| 330319 | 5932415 | Plus | 49095-50132 |
| 328974 | 5868520 | Plus | 31557-31668 |
| 328981 | 5868527 | Minus | 105677-105764 |
| 328989 | 5868535 | Plus | 182088-182198 |
| 330363 | 3126882 | Minus | 61838-61901 |
| 330370 | 6580495 | Plus | 10826-11669 |
| 329041 | 5868564 | Plus | 141592-141785 |
| 329078 | 5868597 | Plus | 326798-326860 |
| 329097 | 5868624 | Plus | 12002-12170 |
| 329107 | 5868626 | Plus | 101063-101190 |
| 329114 | 5868650 | Minus | 23792-23910 |
| 329116 | 5868650 | Minus | 43389-43493 |
| 329164 | 5868691 | Plus | 62305-62517 |
| 329187 | 5868713 | Plus | 29909-30175 |
| 329201 | 5868718 | Plus | 79266-79539 |
| 329221 | 5868727 | Minus | 105837-105894 |
| 329246 | 5868732 | Minus | 250541-250792 |
| 329254 | 5868733 | Plus | 4133-4214 |
| 329326 | 5868806 | Plus | 155884-155992 |
| 329330 | 5868806 | Minus | 340278-340403 |
| 329382 | 5868868 | Plus | 41401-41655 |
| 329384 | 5868869 | Minus | 116524-116662 |
| 329386 | 6004484 | Plus | 160502-161110 |
| 329140 | 6017060 | Plus | 290842-290905 |
| 329182 | 6056331 | Minus | 662206-663423 |
| 329018 | 6249620 | Plus | 103950-104034 |
| 329319 | 6381976 | Plus | 721390-721470 |
| 329392 | 6478815 | Plus | 109786-109854 |
| 329029 | 6525302 | Plus | 281445-282490 |
| 329401 | 6682544 | Plus | 21342-24014 |
| 329406 | 6682547 | Plus | 47249-47395 |
| 329411 | 6682549 | Minus | 84558-84835 |
| 329429 | 5868882 | Minus | 97008-97091 |
| 329436 | 5868883 | Plus | 230265-230528 |
| 329464 | 6456788 | Minus | 4437-4538 |
| Table 1-20B, shows the genomic positioning for those pkeys lacking unigene ID’s and accession numbers in Tables 1-20. For each predicted exon, we have listed the | | | |
| #genomic sequence source used for prediction. Nucleotide locations of each predicted exon are also listed. | | | |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 21
310 GENES UP-REGULATED IN COLON CANCER DERIVED LIVER
METASTASES COMPARED TO NORMAL COLON TISSUE

| Pkey | ExAccn | UnigeneID | Unigene Title | R1 |
|---|---|---|---|---|
| 446619 | AU076643 | Hs.313 | secreted phosphoprotein 1 (osteopontin, | 26.72 |
| 431958 | X63629 | Hs.2877 | cadherin 3, type 1, P-cadherin (placenta | 16.36 |
| 409041 | AB033025 | Hs.50081 | KIAA1199 protein | 13.94 |
| 444381 | BE387335 | Hs.283713 | ESTs, Weakly similar to S64054 hypotheti | 13.90 |
| 432314 | AA533447 | Hs.312989 | ESTs | 12.24 |
| 428330 | L22524 | Hs.2256 | matrix metalloproteinase 7 (matrilysin, | 11.60 |
| 443162 | T49951 | Hs.9029 | DKFZP434G032 protein | 9.52 |
| 436385 | BE551618 | Hs.144097 | ESTs | 9.20 |
| 418662 | AI801098 | Hs.151500 | ESTs | 9.00 |
| 433312 | AI241331 | Hs.131765 | ESTs, Moderately similar to I38937 DNA/R | 8.90 |
| 412093 | BE242691 | Hs.14947 | ESTs | 8.74 |
| 442369 | AI565071 | Hs.159983 | ESTs | 8.40 |
| 426101 | AL049987 | Hs.166361 | Homo sapiens mRNA; cDNA DKFZp564F112 (fr | 8.39 |
| 435937 | AA830893 | Hs.119769 | ESTs | 8.22 |
| 452281 | T93500 | Hs.28792 | Homo sapiens cDNA FLJ11041 fis, clone PL | 8.22 |
| 432572 | AI660840 | Hs.191202 | ESTs, Weakly similar to ALUE_HUMAN !!!! | 7.96 |
| 440524 | R71264 | Hs.16798 | ESTs | 7.94 |
| 424878 | H57111 | Hs.221132 | ESTs | 7.88 |
| 430433 | AA478883 | Hs.273766 | ESTs | 7.82 |
| 410245 | C17908 | Hs.194125 | ESTs | 7.78 |
| 417315 | AI080042 | Hs.336901 | ribosomal protein S24 | 7.76 |
| 430665 | BE350122 | Hs.157367 | ESTs, Weakly similar to I78885 serine/th | 7.76 |
| 432435 | BE218886 | Hs.282070 | ESTs | 7.74 |
| 426818 | AA554827 | Hs.289115 | DKFZp434A0131 protein | 7.58 |
| 419145 | N99638 | | gb: za39g11.r1 Soares fetal liver spleen | 7.56 |
| 444838 | AV651680 | Hs.208558 | ESTs | 7.54 |
| 428046 | AW812795 | Hs.155381 | ESTs, Moderately similar to I38022 hypot | 7.48 |
| 446682 | AW205632 | Hs.211198 | ESTs | 7.26 |
| 421221 | AW276914 | Hs.326714 | Homo sapiens clone IMAGE: 713177, mRNA se | 7.19 |
| 440116 | AI798851 | Hs.283108 | hemoglobin, gamma G | 7.12 |
| 450230 | AW016607 | Hs.201582 | ESTs | 7.08 |
| 456332 | AA228357 | | gb: nc39d05.r1 NCI_CGAP_Pr2 Homo sapiens | 7.04 |
| 421814 | L12350 | Hs.108623 | thrombospondin 2 | 6.89 |
| 440774 | AI420611 | Hs.127832 | ESTs | 6.86 |
| 428065 | AI634046 | Hs.157313 | ESTs | 6.78 |
| 422330 | D30783 | Hs.115263 | epiregulin | 6.72 |
| 413950 | AA249096 | Hs.32793 | ESTs | 6.67 |
| 438011 | BE466173 | Hs.145696 | splicing factor (CC1.3) | 6.62 |
| 421057 | T58283 | Hs.10450 | Homo sapiens cDNA: FLJ22063 fis, clone H | 6.58 |
| 428698 | AA852773 | Hs.334838 | KIAA1866 protein | 6.40 |
| 408806 | AW847814 | Hs.289005 | Homo sapiens cDNA: FLJ21532 fis, clone C | 6.38 |
| 425787 | AA363867 | Hs.155029 | ESTs | 6.38 |
| 435812 | AA700439 | Hs.188490 | ESTs | 6.32 |
| 448974 | AL049390 | Hs.22689 | Homo sapiens mRNA; cDNA DKFZp586O1318 (f | 6.28 |
| 418875 | W19971 | Hs.233459 | ESTs | 6.22 |
| 407284 | AI539227 | Hs.214039 | hypothetical protein FLJ23556 | 6.17 |
| 408243 | Y00787 | Hs.624 | interleukin 8 | 6.12 |
| 434936 | AI285970 | Hs.183817 | ESTs | 6.12 |
| 412088 | AI689496 | Hs.108932 | ESTs | 6.04 |
| 450377 | AB033091 | Hs.74313 | KIAA1265 protein | 6.00 |
| 407618 | AW054922 | Hs.53478 | Homo sapiens cDNA FLJ12366 fis, clone MA | 5.98 |
| 408296 | AL117452 | Hs.44155 | DKFZP586G1517 protein | 5.94 |
| 456999 | AA319798 | Hs.298581 | eukaryotic translation elongation factor | 5.90 |
| 432559 | AW452948 | Hs.257631 | ESTs | 5.88 |
| 423349 | AF010258 | Hs.127428 | homeo box A9 | 5.84 |
| 436100 | AA704806 | Hs.143842 | ESTs, Weakly similar to 2004399A chromos | 5.84 |
| 453204 | R10799 | Hs.191990 | ESTs | 5.84 |
| 429183 | AB014604 | Hs.197955 | KIAA0704 protein | 5.78 |
| 427882 | AA640987 | Hs.193767 | ESTs | 5.72 |
| 447033 | AI357412 | Hs.157601 | ESTs | 5.70 |
| 428054 | AI948688 | Hs.266619 | ESTs | 5.66 |
| 414504 | AW069181 | Hs.115175 | sterile-alpha motif and leucine zipper c | 5.64 |
| 442806 | AW294522 | Hs.149991 | ESTs | 5.64 |
| 418259 | AA215404 | Hs.137289 | ESTs | 5.60 |
| 434963 | AW974957 | Hs.288719 | Homo sapiens cDNA FLJ12142 fis, clone MA | 5.60 |
| 419999 | AI760942 | Hs.191754 | ESTs | 5.58 |
| 431749 | AL049263 | Hs.306292 | Homo sapiens mRNA; cDNA DKFZp564F133 (fr | 5.58 |
| 422790 | AA809875 | Hs.25933 | ESTs | 5.56 |
| 440980 | AL042005 | Hs.1117 | tripeptidyl peptidase II | 5.48 |
| 432451 | AW972771 | Hs.292471 | ESTs, Weakly similar to ALU1_HUMAN ALU S | 5.46 |
| 438578 | AA811244 | Hs.164168 | ESTs | 5.44 |
| 410467 | AF102546 | Hs.63931 | dachshund (Drosophila) homolog | 5.42 |
| 426317 | AA312350 | Hs.169294 | transcription factor 7 (T-cell specific, | 5.42 |
| 450164 | AI239923 | Hs.30098 | ESTs | 5.40 |
| 438899 | AF085833 | Hs.135624 | ESTs | 5.38 |
| 432945 | AL043683 | Hs.8173 | hypothetical protein FLJ10803 | 5.36 |
| 437176 | AW176909 | Hs.42346 | calcineurin-binding protein calsarcin-1 | 5.34 |
| 419829 | AI924228 | Hs.115185 | ESTs, Moderately similar to PC4259 ferri | 5.33 |
| 407966 | AA295052 | Hs.38516 | Homo sapiens, clone MGC:15887, mRNA, com | 5.30 |
| 447342 | AI199268 | Hs.19322 | Homo sapiens, Similar to RIKEN cDNA 2010 | 5.26 |
| 419682 | H13139 | Hs.92282 | paired-like homeodomain transcription fa | 5.26 |
| 421097 | AI280112 | Hs.125232 | Homo sapiens cDNA FLJ13266 fis, clone OV | 5.22 |
| 443373 | AI792868 | Hs.135365 | ESTs | 5.22 |
| 412059 | AA317962 | Hs.249721 | ESTs, Moderately similar to PC4259 ferri | 5.21 |
| 443651 | W22152 | Hs.282929 | ESTs | 5.21 |
| 411274 | NM_002776 | Hs.69423 | kallikrein 10 | 5.17 |
| 421999 | U50535 | Hs.110630 | Human BRCA2 region, mRNA sequence CG006 | 5.17 |
| 426981 | AL044675 | Hs.173081 | KIAA0530 protein | 5.14 |
| 431319 | AA873350 | Hs.302232 | ESTs | 5.10 |
| 434966 | AA657494 | | gb: nt66f04.s1 NCI_CGAP_Pr3 Homo sapiens | 5.10 |
| 418830 | BE513731 | Hs.88959 | hypothetical protein MGC4816 | 5.08 |
| 428290 | AI932995 | Hs.183475 | Homo sapiens clone 25061 mRNA sequence | 5.07 |
| 408784 | AW971350 | Hs.63386 | ESTs | 5.04 |
| 411975 | AI916058 | Hs.144583 | ESTs | 5.02 |
| 409760 | AA302840 | | gb: EST10534 Adipose tissue, white I Homo | 4.97 |
| 420717 | AA284447 | Hs.271887 | ESTs | 4.96 |
| 417035 | AA192455 | Hs.22968 | Homo sapiens clone IMAGE: 451939, mRNA se | 4.95 |
| 434442 | AA737415 | Hs.152826 | ESTs | 4.94 |
| 441328 | AI982794 | Hs.159473 | ESTs | 4.92 |
| 438962 | BE046594 | | gb: hn41c11.x1 NCI_CGAP_RDF2 Homo sapiens | 4.92 |
| 451277 | AK001123 | Hs.26176 | hypothetical protein FLJ10261 | 4.92 |
| 438406 | BE273296 | Hs.254467 | Homo sapiens cDNA FLJ13255 fis, clone OV | 4.90 |
| 424950 | AA602917 | Hs.156974 | ESTs | 4.88 |
| 436823 | AW749865 | Hs.293645 | ESTs, Weakly similar to I38022 hypotheti | 4.87 |
| 444783 | AK001468 | Hs.62180 | anillin (Drosophila Scraps homolog), act | 4.82 |
| 444301 | AK000136 | Hs.10760 | asporin (LRR dass 1) | 4.80 |
| 445390 | AI222165 | Hs.144923 | ESTs | 4.80 |
| 439608 | AW864696 | Hs.301732 | hypothetical protein MGC5306 | 4.78 |
| 450506 | NM_004460 | Hs.418 | fibroblast activation protein, alpha | 4.78 |
| 432682 | AI376400 | Hs.159588 | ESTs | 4.76 |
| 426086 | T94907 | Hs.188572 | ESTs | 4.76 |
| 435981 | H74319 | Hs.188620 | ESTs | 4.74 |
| 432340 | AA534222 | | gb: nj21d02.s1 NCI_CGAP_AA1 Homo sapiens | 4.72 |
| 435756 | AI418466 | Hs.33665 | ESTs | 4.72 |
| 447982 | H22953 | Hs.137551 | ESTs | 4.72 |
| 449509 | AA001615 | Hs.84561 | ESTs | 4.72 |
| 407946 | AA226495 | Hs.154292 | ESTs | 4.70 |
| 426215 | AW963419 | Hs.155223 | stanniocalcin 2 | 4.70 |
| 414783 | AW069569 | Hs.278270 | unactive progesterone receptor, 23 kD | 4.68 |
| 417601 | NM_014735 | Hs.82292 | KIAA0215 gene product | 4.68 |
| 438401 | AW075485 | Hs.286049 | phosphoserine aminotransferase | 4.68 |
| 449032 | AA045573 | Hs.22900 | nuclear factor (erythroid-derived 2)-lik | 4.68 |
| 426501 | AW043782 | Hs.293616 | ESTs | 4.67 |
| 409024 | AW883529 | Hs.173830 | ESTs, Weakly similar to ALU7_HUMAN ALU S | 4.67 |
| 439848 | AW979249 | | gb: EST391359 MAGE resequences, MAGP Homo | 4.66 |
| 424762 | AL119442 | Hs.183684 | eukaryotic translation initiation factor | 4.66 |
| 442007 | AA301116 | Hs.142838 | nucleolar phosphoprotein Nopp34 | 4.62 |
| 409632 | W74001 | Hs.55279 | serine (or cysteine) proteinase inhibito | 4.62 |
| 432409 | AA806538 | Hs.130732 | KIAA1575 protein | 4.60 |
| 452220 | BE158006 | Hs.212296 | ESTs | 4.60 |
| 442577 | AA292998 | Hs.163900 | ESTs | 4.58 |
| 434001 | AW950905 | Hs.3697 | serine (or cysteine) proteinase inhibito | 4.58 |
| 414271 | AK000275 | Hs.75871 | protein kinase C binding protein 1 | 4.58 |
| 433854 | AA610649 | Hs.333239 | ESTs | 4.56 |
| 431315 | AW972227 | Hs.163986 | Homo sapiens cDNA: FLJ22765 fis, clone K | 4.53 |
| 434220 | AI174777 | Hs.283039 | Homo sapiens PRO2492 mRNA, complete cds | 4.50 |
| 457752 | AI821270 | Hs.285643 | Homo sapiens cDNA FLJ14364 fis, clone HE | 4.50 |
| 449941 | AW450536 | Hs.209260 | ESTs | 4.48 |
| 415116 | AA160363 | Hs.269956 | ESTs | 4.47 |
| 414386 | X00442 | Hs.75990 | haptoglobin | 4.47 |
| 422956 | BE545072 | Hs.122579 | hypothetical protein FLJ10461 | 4.44 |
| 423974 | AL118754 | | gb: DKFZp761P1910_r1 761 (synonym: hamy2) | 4.44 |
| 449618 | AI076459 | Hs.15978 | KIAA1272 protein | 4.44 |
| 428279 | AA425310 | Hs.155768 | ESTs, Weakly similar to A47582 B-4 cell gr | 4.42 |
| 430573 | AA744550 | Hs.136345 | ESTs | 4.42 |
| 430929 | AA489166 | Hs.156933 | ESTs | 4.40 |
| 433530 | BE349534 | Hs.281789 | ESTs | 4.40 |
| 446099 | T93096 | Hs.17126 | hypothetical protein MGC15912 | 4.40 |
| 447082 | T85314 | Hs.42644 | thioredoxin-like | 4.39 |
| 407168 | R45175 | Hs.117183 | ESTs | 4.38 |
| 417067 | AJ001417 | Hs.81086 | solute carrier family 22 (extraneuronal | 4.38 |
| 408380 | AF123050 | Hs.44532 | diubiquitin | 4.36 |
| 431379 | AA504264 | Hs.182937 | peptidylprolyl isomerase A (cyclophilin | 4.36 |
| 406671 | AA129547 | Hs.285754 | met proto-oncogene (hepatocyte growth fa | 4.34 |
| 419317 | AA236282 | Hs.172318 | ESTs | 4.32 |
| 450295 | AI766732 | Hs.210628 | ESTs | 4.32 |
| 423578 | AW960454 | Hs.222830 | ESTs | 4.31 |
| 419553 | N34145 | Hs.250614 | ESTs, Moderately similar to ZN91_HUMAN Z | 4.31 |
| 429512 | AA453987 | Hs.144802 | ESTs | 4.30 |
| 426848 | H72531 | Hs.36190 | ESTs | 4.30 |
| 429831 | AA564489 | Hs.137526 | ESTs | 4.30 |
| 433735 | AA608955 | Hs.109653 | ESTs | 4.30 |
| 450546 | AA010200 | Hs.175551 | ESTs | 4.27 |
| 421059 | AI654133 | Hs.30212 | thyroid receptor interacting protein 15 | 4.27 |
| 413243 | AA769266 | Hs.193657 | ESTs | 4.26 |
| 433230 | AW136134 | Hs.220277 | ESTs | 4.22 |
| 439717 | W94472 | Hs.59529 | ESTs, Moderately similar to ALU1_HUMAN A | 4.20 |
| 439382 | AI954880 | Hs.134604 | ESTs | 4.19 |
| 450157 | AW961576 | Hs.60178 | ESTs | 4.17 |
| 451690 | AW451469 | Hs.209990 | ESTs | 4.17 |
| 418661 | NM_001949 | Hs.1189 | E2F transcription factor 3 | 4.16 |
| 443135 | AI376331 | Hs.156103 | ESTs | 4.16 |
| 443148 | AI034357 | Hs.211194 | ESTs, Weakly similar to ALU8_HUMAN ALU S | 4.16 |
| 407765 | AW076027 | Hs.257711 | ESTs, Moderately similar to ALU8_HUMAN A | 4.14 |
| 428825 | AI084336 | Hs.128783 | ESTs, Weakly similar to I38022 hypotheti | 4.14 |
| 447519 | U46258 | Hs.339665 | ESTs | 4.14 |
| 439451 | AF086270 | Hs.278554 | heterochromatin-like protein 1 | 4.12 |
| 450219 | AI826999 | Hs.224624 | ESTs | 4.12 |
| 431451 | AA761378 | Hs.192013 | ESTs | 4.11 |
| 432917 | NM_014125 | Hs.279812 | PRO0327 protein | 4.10 |
| 431328 | AA502999 | Hs.291591 | ESTs | 4.09 |
| 425992 | AA367069 | Hs.100636 | ESTs | 4.08 |
| 404571 | | | | 4.06 |
| 420911 | U77413 | Hs.100293 | O-linked N-acetylglucosamine (GlcNAc) tr | 4.06 |
| 421114 | AW975051 | Hs.293156 | ESTs, Weakly similar to I78885 serine/th | 4.06 |
| 432731 | R31178 | Hs.287820 | fibronectin 1 | 4.06 |
| 433588 | AI056872 | Hs.133386 | ESTs | 4.06 |
| 434658 | AI624436 | Hs.310286 | ESTs | 4.06 |
| 444040 | AF204231 | Hs.182982 | golgin-67 | 4.06 |
| 444984 | H15474 | Hs.132898 | fatty acid desaturase 1 | 4.06 |
| 438543 | AA810141 | Hs.192182 | ESTs | 4.05 |
| 413497 | BE177661 | | gb: RC1-HT0598-020300-011-h02 HT0598 Homo | 4.04 |
| 434575 | AI133446 | Hs.299964 | Homo sapiens clone FLB7723 PRO2055 mRNA, | 4.04 |
| 430256 | AA470152 | Hs.192195 | ESTs | 4.04 |
| 424839 | AA740632 | Hs.120850 | ESTs, Weakly similar to ALU1_HUMAN ALU S | 4.02 |
| 429048 | AI372949 | Hs.44241 | Homo sapiens cDNA: FLJ21447 fis, clone C | 4.02 |
| 449429 | AA054224 | Hs.59847 | ESTs | 4.02 |
| 410762 | AF226053 | Hs.66170 | HSKM-B protein | 4.00 |
| 418876 | AA740616 | | gb: nyg7f11.s1 NCI_CGAP_GCB1 Homo sapiens | 4.00 |
| 425905 | AB032959 | Hs.318584 | novel C3HC4 type Zinc finger (ring finge | 4.00 |
| 429500 | X78565 | Hs.289114 | hexabrachion (tenascin C, cytotactin) | 4.00 |
| 431393 | AW971493 | Hs.134269 | ESTs, Highly similar to cytokine recepto | 4.00 |
| 435008 | AF150262 | Hs.162898 | ESTs | 4.00 |
| 431361 | AW971375 | Hs.292921 | ESTs | 3.97 |
| 444816 | Z48633 | Hs.283742 | H. sapiens, mRNA for retrotransposon | 3.96 |
| 434701 | AA460479 | Hs.321707 | KIAA0742 protein | 3.96 |
| 413886 | AW958264 | Hs.103832 | similar to yeast Upf3, variant B | 3.95 |
| 424905 | NM_002497 | Hs.153704 | NIMA (never in mitosis gene a)-related k | 3.92 |
| 428479 | Y00272 | Hs.184572 | cell division cycle 2, G1 to S and G2 to | 3.91 |
| 435714 | AA699325 | Hs.269880 | ESTs | 3.86 |
| 447514 | AI809314 | Hs.208501 | ESTs, Weakly similar to B34087 hypotheti | 3.86 |
| 453818 | BE256832 | Hs.10711 | hypothetical protein FLJ13449 | 3.85 |
| 433586 | T85301 | | gb: yd78d06.s1 Soares fetal liver spleen | 3.85 |
| 440638 | AI376551 | | gb: te64e10.x1 Soares_NFL_T_GBC_S1 Homo s | 3.85 |
| 417819 | AI253112 | Hs.133540 | ESTs | 3.84 |
| 409596 | BE244200 | Hs.55075 | KIAA0410 gene product | 3.83 |
| 423129 | L44396 | Hs.124106 | Homo sapiens cDNA FLJ11941 fis, clone HE | 3.83 |
| 453884 | AA355925 | Hs.36232 | KIAA0186 gene product | 3.83 |
| 431193 | AW749505 | Hs.296770 | KIAA1719 protein | 3.81 |
| 409262 | AK000631 | Hs.52256 | hypothetical protein FLJ20624 | 3.80 |
| 425568 | AW963118 | Hs.161784 | ESTs | 3.78 |
| 441085 | AW136551 | Hs.181245 | Homo sapiens cDNA FLJ12532 fis, clone NT | 3.77 |
| 428079 | AA421020 | Hs.208919 | ESTs | 3.77 |
| 412490 | AW803564 | Hs.288850 | Homo sapiens cDNA: FLJ22528 fis, clone H | 3.76 |
| 435354 | AA678267 | Hs.117115 | ESTs | 3.75 |
| 436535 | AW295687 | Hs.254420 | ESTs | 3.74 |
| 420439 | AW270041 | Hs.193053 | eukaryotic translation initiation factor | 3.72 |
| 436090 | AI640635 | Hs.116468 | EST | 3.71 |
| 416265 | AA177088 | Hs.190065 | ESTs | 3.70 |
| 417715 | AW969587 | Hs.86366 | ESTs | 3.67 |
| 435677 | AA694142 | Hs.293726 | ESTs, Weakly similar to TSGA RAT TESTIS | 3.67 |
| 438607 | AW080237 | Hs.252884 | ESTs | 3.66 |
| 408194 | AA601038 | Hs.191797 | ESTs, Weakly similar to S65657 alpha-1C- | 3.65 |
| 417211 | T97617 | Hs.269092 | ESTs | 3.60 |
| 435538 | AB011540 | Hs.4930 | low density lipoprotein receptor-related | 3.59 |
| 410390 | AA876905 | Hs.125286 | ESTs | 3.58 |
| 438818 | AW979008 | Hs.222487 | ESTs | 3.57 |
| 431416 | AA532718 | Hs.178604 | ESTs | 3.57 |
| 433517 | AW022133 | Hs.189838 | ESTs | 3.56 |
| 428355 | BE256452 | Hs.2257 | vitronectin (serum spreading factor, som | 3.56 |
| 432954 | AI076345 | Hs.214199 | ESTs | 3.53 |
| 434466 | AB037829 | Hs.3862 | regulator of nonsense transcripts 2; DKF | 3.53 |
| 421933 | R98881 | Hs.109655 | sex comb on midleg (Drosophila)-like 1 | 3.52 |
| 422082 | AA016188 | Hs.111244 | hypothetical protein | 3.52 |
| 437135 | AL038624 | Hs.208752 | ESTs, Weakly similar to ALU8_HUMAN ALU S | 3.49 |
| 424723 | BE409813 | Hs.152337 | protein arginine N-methyltransferase 3(h | 3.49 |
| 434280 | BE005398 | | gb: CM1-BN0116-150400-189-h02 BN0116 Homo | 3.49 |
| 407289 | AA135159 | Hs.203349 | Homo sapiens cDNA FLJ12149 fis, clone MA | 3.48 |
| 417670 | R07785 | | gb: yf15c06.r1 Soares fetal liver spleen | 3.48 |
| 431615 | AW295859 | Hs.235860 | ESTs | 3.48 |
| 429355 | AW973253 | Hs.292689 | ESTs | 3.45 |
| 430068 | AA464964 | | gb: zx80f10.s1 Soares ovary tumor NbHOT H | 3.45 |
| 432929 | AW207166 | Hs.191265 | ESTs | 3.44 |
| 437763 | AA469369 | Hs.5831 | tissue inhibitor of metalloproteinase 1 | 3.44 |
| 445674 | BE410347 | Hs.13063 | transcription factor CA150 | 3.42 |
| 408113 | T82427 | Hs.194101 | Homo sapiens cDNA: FLJ20869 fis, clone A | 3.42 |
| 408908 | BE296227 | Hs.250822 | serine/threonine kinase 15 | 3.41 |
| 432235 | AA531129 | Hs.190297 | ESTs | 3.41 |
| 453985 | N44545 | Hs.251865 | ESTs | 3.41 |
| 415736 | AA827082 | Hs.291872 | ESTs | 3.38 |
| 430220 | BE378277 | Hs.152230 | ESTs | 3.37 |
| 426510 | AW861225 | Hs.194637 | BANP homolog, SMAR1 homolog | 3.37 |
| 412104 | AW205197 | Hs.240951 | Homo sapiens, Similar to RIKEN cDNA 2210 | 3.36 |
| 411573 | AB029000 | Hs.70823 | KIAA1077 protein | 3.33 |
| 413816 | AW958181 | Hs.189998 | ESTs | 3.32 |
| 428057 | AI343641 | Hs.185798 | ESTs | 3.32 |
| 436280 | AI690734 | Hs.131740 | Homo sapiens cDNA: FLJ22562 fis, clone H | 3.31 |
| 449365 | AW968261 | Hs.118913 | ESTs, Moderately similar to T46371 hypot | 3.31 |
| 440659 | AF134160 | Hs.7327 | claudin 1 | 3.30 |
| 436110 | AA704899 | Hs.291651 | ESTs, Weakly similar to I38022 hypotheti | 3.29 |
| 433862 | D86960 | Hs.3610 | KIAA0205 gene product | 3.29 |
| 424624 | AB032947 | Hs.151301 | Ca2+ dependent activator protein for secr | 3.29 |
| 439955 | AW203959 | Hs.149532 | ESTs | 3.28 |
| 417333 | AL157545 | Hs.42179 | bromodomain and PHD finger containing, 3 | 3.28 |
| 436150 | AW510927 | Hs.125243 | ESTs | 3.27 |
| 414900 | AW452420 | Hs.248678 | ESTs | 3.26 |
| 439349 | AI660898 | Hs.195602 | ESTs | 3.25 |
| 428255 | AI627478 | Hs.187670 | ESTs | 3.24 |
| 436217 | T53925 | Hs.107 | fibrinogen-like 1 | 3.24 |
| 429083 | Y09397 | Hs.227817 | BCL2-related protein A1 | 3.24 |
| 422244 | Y08890 | Hs.113503 | karyopherin (importin) beta 3 | 3.22 |
| 430178 | AW449612 | Hs.152475 | ESTs | 3.21 |
| 413810 | AW197644 | Hs.19107 | ESTs | 3.20 |
| 428728 | NM_016625 | Hs.191381 | hypothetical protein | 3.20 |
| 437151 | AA745618 | Hs.194637 | BANP homolog, SMAR1 homolog | 3.19 |
| 427051 | BE178110 | Hs.173374 | Homo sapiens cDNA FLJ10500 fis, clone NT | 3.19 |
| 438378 | AW970529 | Hs.86434 | hypothetical protein FLJ21816 | 3.19 |
| 439943 | AW083789 | Hs.124620 | ESTs | 3.18 |
| 439280 | AI125436 | Hs.48752 | ESTs | 3.18 |
| 452336 | AA960961 | Hs.305953 | zinc finger protein 83 (HPF1) | 3.17 |
| 433713 | AW976511 | Hs.112592 | ESTs | 3.16 |
| 414998 | NM_002543 | Hs.77729 | oxidised low density lipoprotein (lectin | 3.14 |
| 407328 | AA508857 | Hs.187748 | ESTs, Weakly similar to ALU1_HUMAN ALU S | 3.14 |
| 432722 | AA830532 | Hs.326150 | ESTs | 3.14 |
| 419457 | AA243146 | Hs.209334 | ESTs, Moderately similar to S23A_HUMAN P | 3.11 |
| 449987 | AW079749 | Hs.184719 | ESTs, Weakly similar to ALU1_HUMAN ALU S | 3.11 |
| 418522 | AA605038 | Hs.7149 | Homo sapiens cDNA: FLJ21950 fis, clone H | 3.09 |
| 409969 | AW514668 | Hs.194258 | ESTs, Moderately similar to ALU5_HUMAN A | 3.08 |
| 436299 | AK000767 | Hs.5111 | hypothetical protein FLJ20729 | 3.08 |
| 406687 | M31126 | Hs.272620 | pregnancy specific beta-1-glycoprotein 9 | 3.07 |
| 408242 | AA251594 | Hs.43913 | PIBF1 gene product | 3.07 |
| 444614 | R44284 | Hs.2730 | heterogeneous nuclear ribonucleoprotein | 3.06 |
| 459407 | N92114 | | gb: za22h11.r1 Soares fetal liver spleen | 3.05 |
| 433972 | AI878910 | Hs.3688 | cisplatin resistance-associated overexpr | 3.04 |
| 427704 | AW971063 | Hs.292882 | ESTs | 3.03 |
| 440255 | AI932285 | Hs.160569 | ESTs | 3.03 |
| 424542 | AI860558 | Hs.272009 | ESTs, Weakly similar to ALU2_HUMAN ALU S | 3.03 |
| 413822 | R08950 | Hs.272044 | ESTs, Weakly similar to ALU1_HUMAN ALU S | 3.02 |
| 433944 | AL117518 | Hs.3686 | KIAA0978 protein | 3.01 |
| 440428 | BE560954 | | gb: 601347719F1 NIH_MGC_8 Homo sapiens cD | 3.00 |
| Table 21 shows 310 genes up-regulated in colon cancer derived liver metastases compared to normal colon tissue. These were selected from 59680 probesets on the Affymetrix/Eos HuO3 GeneChip array such that the ratio of “average” colon cancer derived liver metastases to | | | | |
| #“average” normal colon tissues was greater than or equal to 3.0. The “average” colon cancer derived liver metastases level was set to the 50th percentile. The “average” normal colon tissue level was set to the 50th percentile. | | | | |

Pkey: Unique Eos probeset identifier number
ExAccn: Exemplar Accession number, Genbank accession number
UnigeneID: Unigene number
Unigene Title: Unigene gene title
RI: Genes up mets vs normal

TABLE 21A

| Pkey | CAT Number | Accession |
|---|---|---|
| 409760 | 115373_1 | AA302840 T93016 T92950 AA077551 |
| 413497 | 1373771_1 | BE177661 H06215 BE144709 BE144829 |
| 417670 | 1692163_1 | R07785 T85948 T86972 |
| 418876 | 179960_1 | AA740616 AA654854 AA229923 |
| 419145 | 182217_1 | N99638 AW973750 AA328271 H90994 |
| | | AA558020 AA234435 N59599 R94815 |
| 423974 | 233842_1 | AL118754 AA333202 H38001 |
| 430068 | 312849_1 | AA464964 M85405 AA947566 |
| 432340 | 345248_1 | AA534222 AA632632 T81234 |
| 433586 | 370470_1 | T85301 AW517087 AA601054 BE073959 |
| 434280 | 382816_1 | BE005398 AA628622 AA994155 |
| 434966 | 396504_1 | AA657494 AI582663 AI581639 |
| 438962 | 467390_1 | BE046594 BE046667 AA828585 AI207343 |
| 439848 | 477806_1 | AW979249 D63277 AA846968 |
| 440428 | 49370_−1 | BE560954 |
| 440638 | 499025_1 | AI376551 T87714 AA897445 |
| 456332 | 179104_1 | AA228357 AW841786 AW841716 |
| Table 21A shows the accession numbers for those pkeys lacking unigeneID's for Table 21A. For each probeset we have listed the gene cluster number from which the oligonucleotides were designed. Gene clusters were compiled using sequences derived from Genbank ESTs and mRNAs. These sequences were clustered based on sequence similarity using Clustering | | |
| # and Alignment Tools (DoubleTwist, Oakland California). The Genbank accession numbers for sequences comprising each cluster are listed in the “Accession” column. | | |

Pkey: Unique Eos probeset identifier number
CAT number: Gene cluster number
Accession: Genbank accession numbers

TABLE 21B

| Pkey | Ref | Strand | Nt_position |
|---|---|---|---|
| 404571 | 7249169 | Minus | 112450-112648 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 22
177 GENES DOWN-REGULATED IN COLON CANCER DERIVED
LIVER METASTASES COMPARED TO NORMAL COLON TISSUE

| Pkey | ExAccn | UnigeneID | Unigene Title | R1 |
|---|---|---|---|---|
| 425196 | AL037915 | Hs.155097 | carbonic anhydrase II | 0.03 |
| 414522 | AW518944 | Hs.76325 | step II splicing factor SLU7 | 0.03 |
| 409153 | W03754 | Hs.50813 | hypothetical protein FLJ20022 | 0.03 |
| 452594 | AU076405 | Hs.29981 | solute carrier family 26 (sulfate transp | 0.03 |
| 424326 | NM_014479 | Hs.145296 | disintegrin protease | 0.04 |
| 414798 | AI286323 | Hs.97411 | hypothetical protein MGC12335 | 0.04 |
| 432150 | AK000224 | Hs.272789 | hypothetical protein FLJ20217 | 0.04 |
| 425206 | NM_002153 | Hs.155109 | hydroxysteroid (17-beta) dehydrogenase 2 | 0.05 |
| 437145 | AF007216 | Hs.5462 | solute carrier family 4, sodium bicarbon | 0.05 |
| 447513 | AW955776 | Hs.313500 | ESTs, Moderately similar to ALU7_HUMAN A | 0.05 |
| 414807 | AI738616 | Hs.77348 | hydroxyprostaglandin dehydrogenase 15-(N | 0.06 |
| 428934 | AF039401 | Hs.194659 | chloride channel, calcium activated, fam | 0.06 |
| 432251 | AW972983 | Hs.232165 | polycythemia rubra vera 1; cell surface | 0.07 |
| 431727 | AW293464 | Hs.162031 | ESTs | 0.07 |
| 421515 | Y11339 | Hs.105352 | GalNAc alpha-2, 6-sialyltransferase I, I | 0.07 |
| 414555 | N98569 | Hs.76422 | phospholipase A2, group IIA (platelets, | 0.08 |
| 412047 | AA934589 | Hs.49696 | ESTs | 0.08 |
| 412056 | T28160 | Hs.778 | guanylate cyclase activator 1B (retina) | 0.08 |
| 422440 | NM_004812 | Hs.116724 | aldo-keto reductase family 1, member B10 | 0.08 |
| 450684 | AA872605 | Hs.25333 | interleukin 1 receptor, type II | 0.09 |
| 418935 | T28499 | Hs.89485 | carbonic anhydrase IV | 0.09 |
| 433658 | L03678 | Hs.156110 | immunoglobulin kappa constant | 0.09 |
| 422260 | AA315993 | Hs.105484 | regenerating gene type IV | 0.09 |
| 433336 | AF017986 | Hs.31386 | secreted frizzled-related protein 2 | 0.09 |
| 426784 | U03749 | Hs.172216 | chromogranin A (parathyroid secretory pr | 0.09 |
| 441888 | AI733306 | Hs.128071 | hypothetical protein FLJ21302 | 0.10 |
| 440624 | AF017987 | Hs.7306 | secreted frizzled-related protein 1 | 0.10 |
| 420929 | AI694143 | Hs.296251 | programmed cell death 4 | 0.10 |
| 429970 | AK000072 | Hs.227059 | chloride channel, calcium activated, fam | 0.10 |
| 417233 | W25005 | Hs.24395 | small inducible cytokine subfamily B (Cy | 0.10 |
| 414802 | AI793107 | Hs.27018 | Ris | 0.10 |
| 424566 | M16801 | Hs.1790 | nuclear receptor subfamily 3, group C, m | 0.11 |
| 421996 | AW583807 | Hs.1460 | glucagon | 0.11 |
| 423371 | AU076819 | Hs.1650 | solute carrier family 26, member 3 | 0.11 |
| 406741 | AA058357 | Hs.74466 | carcinoembryonic antigen-related cell ad | 0.11 |
| 414176 | BE140638 | Hs.75794 | endothelial differentiation, lysophospha | 0.11 |
| 408741 | M73720 | Hs.646 | carboxypeptidase A3 (mast cell) | 0.11 |
| 424527 | AW138558 | Hs.267158 | ESTs, Weakly similar to I54374 gene NF2 | 0.12 |
| 426682 | AV660038 | Hs.2056 | UDP glycosyltransferase 1 family, polype | 0.12 |
| 453967 | AW009077 | Hs.232947 | ESTs | 0.12 |
| 425920 | AL049977 | Hs.162209 | claudin 8 | 0.13 |
| 408134 | AK000184 | Hs.42945 | acid sphingomyelinase-like phosphodieste | 0.13 |
| 457407 | AA505035 | Hs.195651 | ESTs | 0.13 |
| 446500 | U78093 | Hs.15154 | sushi-repeat-containing protein, X chrom | 0.14 |
| 422487 | AJ010901 | Hs.198267 | mucin 4, tracheobronchial | 0.14 |
| 409196 | NM_001874 | Hs.334873 | carboxypeptidase M | 0.14 |
| 416426 | AA180256 | Hs.210473 | Homo sapiens cDNA FLJ14872 fis, clone PL | 0.14 |
| 406636 | L12064 | | gb: Homo sapiens (clone WR4.12VL) anti-th | 0.14 |
| 457982 | AW856093 | Hs.183617 | ESTs | 0.14 |
| 407744 | AB020629 | Hs.38095 | ATP-binding cassette, sub-family A (ABC1 | 0.14 |
| 430378 | Z29572 | Hs.2556 | tumor necrosis factor receptor superfami | 0.14 |
| 424885 | AI333771 | Hs.82204 | ESTs | 0.14 |
| 423555 | AW958201 | Hs.178589 | hepatocellular carcinoma antigen gene 52 | 0.14 |
| 444237 | AA336878 | Hs.9842 | Human DNA sequence from clone RP4-788L20 | 0.14 |
| 445848 | AA774824 | Hs.13377 | Homo sapiens clone 23649 and 23755 unkno | 0.14 |
| 451062 | AL110125 | Hs.25910 | Homo sapiens mRNA; cDNA DKFZp564C1416 (f | 0.14 |
| 436485 | X59135 | Hs.156110 | immunoglobulin kappa constant | 0.14 |
| 423655 | AA722425 | Hs.182785 | ESTs, Moderately similar to 1207289A rev | 0.15 |
| 417332 | AW972717 | Hs.288462 | hypothetical protein FLJ21511 | 0.15 |
| 427506 | AK000134 | Hs.179100 | hypothetical protein FLJ20127 | 0.15 |
| 430712 | AW044647 | Hs.196284 | ESTs | 0.15 |
| 421666 | AL035250 | Hs.1408 | endothelin 3 | 0.16 |
| 425692 | D90041 | Hs.155956 | N-acetyltransferase 1 (arylamine N-acety | 0.16 |
| 429412 | NM_006235 | Hs.2407 | POU domain, class 2, associating factor | 0.16 |
| 433745 | AF075320 | Hs.28980 | hypothetical protein FLJ14540 | 0.16 |
| 450085 | AW293791 | Hs.60162 | Homo sapiens cDNA: FLJ21528 fis, clone C | 0.16 |
| 417820 | D87449 | Hs.82635 | UDP-glucuronic acid/UDP-N-acetylgalactos | 0.16 |
| 406722 | H27498 | Hs.293441 | Homo sapiens SNC73 protein (SNC73) mRNA, | 0.16 |
| 426488 | X03350 | Hs.4 | alcohol dehydrogenase 1B (class I), beta | 0.16 |
| 436327 | AA813075 | Hs.120181 | ESTs | 0.16 |
| 408873 | AL0A6017 | Hs.182278 | calmodulin 2 (phosphorylase kinase, delt | 0.16 |
| 429524 | AB033037 | Hs.205293 | KIAA1211 protein | 0.16 |
| 447023 | AA356764 | Hs.17109 | integral membrane protein 2A | 0.17 |
| 424264 | D80400 | Hs.239388 | Human DNA sequence from clone RP1-304B14 | 0.17 |
| 410310 | J02931 | Hs.62192 | coagulation factor III (thromboplastin, | 0.17 |
| 432563 | NM_013261 | Hs.198468 | peroxisome proliferative activated recep | 0.17 |
| 406897 | M57417 | | gb: Homo sapiens mucin (mucin) mRNA, part | 0.17 |
| 451096 | BE383234 | Hs.25925 | Homo sapiens, clone MGC: 15393, mRNA, com | 0.17 |
| 447726 | AL137638 | Hs.19366 | matrilin 2 | 0.17 |
| 409549 | AB029015 | Hs.54886 | phospholipase C, epsilon 2 | 0.17 |
| 433334 | AI927208 | Hs.231958 | matrix metalloproteinase 28 | 0.17 |
| 425849 | AJ000512 | Hs.296323 | serum/glucocorticoid regulated kinase | 0.17 |
| 407360 | X13075 | | gb: Human 2a12 mRNA for kappa-immunoglobu | 0.17 |
| 430627 | U61148 | Hs.247685 | atonal homolog 1 (Drosophila) | 0.17 |
| 418807 | NM_004944 | Hs.88646 | deoxyribonuclease I-like 3 | 0.18 |
| 453399 | Z70295 | Hs.32966 | guanylate cyclase activator 2B (uroguany | 0.18 |
| 422994 | AW891802 | Hs.296276 | ESTs | 0.18 |
| 432134 | AI816782 | Hs.122583 | hypothetical protein FLJ21934 | 0.18 |
| 400417 | X72475 | | | 0.18 |
| 443506 | H10661 | Hs.192124 | ESTs, Weakly similar to I38022 hypotheti | 0.18 |
| 428470 | AC002301 | Hs.184507 | Homo sapiens Chromosome 16 BAC clone CIT | 0.18 |
| 451928 | AI823801 | Hs.30315 | CTCL tumor antigen se57-1 | 0.18 |
| 429576 | BE242628 | Hs.209061 | sudD (suppressor of bimD6, Aspergillus n | 0.18 |
| 422106 | D84239 | Hs.111732 | Fc fragment of IgG binding protein | 0.19 |
| 430304 | AL122071 | Hs.238927 | Homo sapiens mRNA; CDNA DKFZp434H1235 (f | 0.19 |
| 452852 | AK001972 | Hs.30822 | hypothetical protein FLJ11110 | 0.19 |
| 421904 | BE143533 | Hs.109309 | hypothetical protein FLJ20035 | 0.19 |
| 417165 | R80137 | Hs.302738 | Homo sapiens cDNA: FLJ21425 fis, clone C | 0.19 |
| 417771 | AA804698 | Hs.82547 | retinoic acid receptor responder (tazaro | 0.19 |
| 452802 | AU076403 | Hs.323468 | electron-transferring-flavoprotein dehyd | 0.19 |
| 450680 | AF131784 | Hs.25318 | Homo sapiens clone 25194 mRNA sequence | 0.19 |
| 420061 | AW024937 | Hs.29410 | ESTs | 0.19 |
| 426828 | NM_000020 | Hs.172670 | activin A receptor type II-like 1 | 0.19 |
| 408190 | AB032963 | Hs.43577 | ATPase, Class I, type 8B, member 2 | 0.19 |
| 437682 | AA476652 | Hs.94952 | Homo sapiens cDNA: FLJ23371 fis, clone H | 0.19 |
| 449110 | H56112 | | gb: yq95f07.r1 Soares fetal liver spleen | 0.19 |
| 446727 | AB011095 | Hs.16032 | KIAA0523 protein | 0.19 |
| 408395 | BE072425 | Hs.44579 | hypothetical protein FLJ20199 | 0.20 |
| 423541 | AA296922 | Hs.129778 | gastrointestinal peptide | 0.20 |
| 410850 | AW362867 | Hs.302738 | Homo sapiens cDNA: FLJ21425 fis, clone C | 0.20 |
| 412420 | AL035668 | Hs.73853 | bone morphogenetic protein 2 | 0.20 |
| 423942 | AF209704 | Hs.135723 | glycolipid transfer protein | 0.20 |
| 421832 | NM_016098 | Hs.108725 | HSPC040 protein | 0.20 |
| 459046 | AA910339 | Hs.26216 | LOC50627 | 0.20 |
| 421360 | AA297012 | Hs.103839 | erythrocyte membrane protein band 4.1-li | 0.20 |
| 438091 | AW373062 | Hs.83623 | nuclear receptor subfamily 1, group I, m | 0.20 |
| 403047 | | | | 0.20 |
| 421712 | AK000140 | Hs.107139 | hypothetical protein | 0.20 |
| 427333 | AF067797 | Hs.176658 | aquaporin 8 | 0.20 |
| 421964 | X73079 | Hs.288579 | polymeric immunoglobulin receptor | 0.20 |
| 438089 | W05391 | Hs.83623 | nuclear receptor subfamily 1, group I, m | 0.21 |
| 445200 | AA084460 | Hs.12409 | somatostatin | 0.21 |
| 404854 | | | | 0.21 |
| 426390 | AA377299 | Hs.90431 | ESTs | 0.21 |
| 403381 | | | | 0.21 |
| 449833 | R82252 | Hs.106106 | protein kinase (cAMP-dependent, catalyti | 0.21 |
| 457718 | F18572 | Hs.22978 | ESTs, Weakly similar to ALU4_HUMAN ALU S | 0.21 |
| 435730 | AB020635 | Hs.4984 | KIAA0828 protein | 0.21 |
| 431518 | AA743462 | Hs.165337 | ESTs | 0.21 |
| 412589 | R28660 | Hs.24305 | ESTs | 0.21 |
| 432584 | AA928829 | Hs.47099 | hypothetical protein FLJ21212 | 0.21 |
| 426088 | AF038007 | Hs.166196 | ATPase, Class I, type 8B, member 1 | 0.21 |
| 429143 | AA333327 | Hs.197335 | plasma glutamate carboxypeptidase | 0.21 |
| 414429 | R51494 | Hs.71818 | ESTs | 0.22 |
| 439670 | AF088076 | Hs.59507 | ESTs, Weakly similar to AC004858 3 U1 sm | 0.22 |
| 406697 | M21388 | Hs.123017 | Human unproductively rearranged Ig mu-ch | 0.22 |
| 406663 | U24683 | Hs.302063 | immunoglobulin heavy constant mu | 0.22 |
| 407811 | AW190902 | Hs.40098 | cysteine knot superfamily 1, BMP antagon | 0.22 |
| 417880 | BE241595 | Hs.82848 | selectin L (lymphocyte adhesion molecule | 0.22 |
| 430107 | AA465293 | Hs.105069 | ESTs | 0.22 |
| 424273 | W40460 | Hs.144442 | phospholipase A2, group X | 0.22 |
| 419559 | Y07828 | Hs.91096 | ring finger protein | 0.22 |
| 413517 | N76712 | Hs.44829 | ESTs, Weakly similar to I38022 hypotheti | 0.22 |
| 407243 | AA058357 | Hs.74466 | carcinoembryonic antigen-related cell ad | 0.22 |
| 433906 | AI167816 | Hs.43355 | ESTs | 0.22 |
| 446203 | Z47553 | Hs.14286 | flavin containing monooxygenase 5 | 0.22 |
| 403740 | | | | 0.22 |
| 405701 | | | | 0.22 |
| 413554 | AA319146 | Hs.75426 | secretogranin II (chromogranin C) | 0.22 |
| 419577 | L36531 | Hs.91296 | integrin, alpha 8 | 0.23 |
| 451820 | AW058357 | Hs.337353 | ESTs | 0.23 |
| 424897 | D63216 | Hs.153684 | frizzled-related protein | 0.23 |
| 422880 | AF228704 | Hs.121524 | glutathione reductase | 0.23 |
| 430832 | AI073913 | Hs.100686 | ESTs, Weakly similar to JE0350 Anterior | 0.23 |
| 430753 | AI432401 | Hs.2659 | fibrinogen-like 2 | 0.23 |
| 409060 | AI815867 | Hs.50130 | necdin (mouse) homolog | 0.23 |
| 412228 | AW503785 | Hs.73792 | complement component (3d/Epstein Barr vi | 0.24 |
| 414171 | AA360328 | Hs.865 | RAP1A, member of RAS oncogene family | 0.24 |
| 417916 | NM_006416 | Hs.82921 | solute carrier family 35 (CMP.sialic aci | 0.24 |
| 414589 | AA149791 | Hs.68864 | ESTs, Weakly similar to phosphatidylseri | 0.24 |
| 427167 | AI239607 | Hs.99196 | hypothetical protein MGC11324 | 0.24 |
| 440630 | BE561430 | Hs.239388 | Human DNA sequence from clone RP1-304B14 | 0.24 |
| 423044 | AA320829 | Hs.97266 | protocadherin 18 | 0.24 |
| 441931 | BE564830 | Hs.23744 | hypothetical protein FLJ12899 | 0.24 |
| 443060 | D78874 | Hs.8944 | procollagen C-endopeptidase enhancer 2 | 0.24 |
| 405441 | | | | 0.24 |
| 407241 | M34516 | | gb: Human omega light chain protein 14.1 | 0.24 |
| 415165 | AW887604 | Hs.78065 | complement component 7 | 0.24 |
| 426447 | AV655843 | Hs.169919 | electron-transfer-flavoprotein, alpha po | 0.24 |
| 410748 | BE383816 | Hs.12532 | chromosome 1 open reading frame 21 | 0.24 |
| 436032 | AA150797 | Hs.109276 | latexin protein | 0.24 |
| 414256 | AW410035 | Hs.75862 | MAD (mothers against decapentaplegic, Dr | 0.24 |
| 414197 | W44877 | Hs.55501 | ESTs | 0.24 |
| 406836 | AW514501 | Hs.156110 | immunoglobulin kappa constant | 0.24 |
| 437083 | AW082597 | Hs.244862 | ESTs | 0.25 |
| 421709 | AA159394 | Hs.107056 | CED-6 protein | 0.25 |
| 426512 | AW511656 | Hs.170177 | Meis1 (mouse) homolog | 0.25 |
| Table 22 shows 177 genes down-regulated in colon cancer derived liver metastases compared to normal colon tissue. These were selected from 59680 probesets on the Affymetrix/Eos Hu03 GeneChip array such that the ratio of “average” colon cancer derived | | | | |
| #liver metastases to “average” normal colon tissues was less than or equal to 0.25. The “average” colon cancer derived liver metastases level was set to the 50th percentile. The “average” normal adult tissue level was set to the 50th percentile. | | | | |

Pkey: Unique Eos probeset identifier number
ExAccn: Exemplar Accession number, Genbank accession number
UnigeneID: Unigene number
Unigene Title: Unigene gene title
R1: Genes down mets vs. normal

TABLE 22A
Table 22A shows the accession numbers for those pkeys lacking
unigeneID's for Tables 21A. For each probeset we have
listed the gene cluster number from which the oligonucleotides
were designed. Gene clusters were compiled using sequences
derived from Genbank ESTs and mRNAs. These sequences were
clustered based on sequence similarity using Clustering and
Alignment Tools (DoubleTwist, Oakland California). The Genbank
accession numbers for sequences comprising each cluster are
listed in the “Accession” column.

| | |
|---|---|
| Pkey: | Unique Eos probeset identifier number |
| CAT number: | Gene cluster number |
| Accession: | Genbank accession numbers |

| Pkey | CAT Number | Accession |
|---|---|---|
| 449110 | 798430_1 | H56112 H58047 AI630710 N58742 |

TABLE 22B

| | |
|---|---|
| Pkey: | Unique number corresponding to an Eos probeset |
| Ref: | Sequence source. The 7 digit numbers in this column |
| | are Genbank Identifier (GI) numbers. “Dunham I. |
| | et al.” refers to the publication entitled |
| | “The DNA sequence of human chromosome 22.” |
| | Dunham I. et al., Nature (1999) 402:489-495. |
| Strand: | Indicates DNA strand from which exons were |
| | predicted. |
| Nt_position: | Indicates nucleotide positions of predicted exons. |

| Pkey | Ref | Strand | Nt_position |
|---|---|---|---|
| 403047 | 3540153 | Minus | 59793-59968 |
| 403381 | 9438267 | Minus | 26009-26178 |
| 403740 | 7630882 | Plus | 86504-87227 |
| 404854 | 7143420 | Plus | 14260-14537 |
| 405441 | 7408124 | Plus | 100952-101283 |
| 405701 | 4263751 | Plus | 93243-93354 |

TABLE 23
175 GENES UP-REGULATED IN COLON CANCER
DERIVED LIVER METASTASES COMPARED TO COLON CANCER
PRIMARY TUMOR SAMPLES CLASSIFIED AS DUKE'S B SURVIVOR
Table 23 shows 175 genes up-regulated in colon cancer derived liver metastases compared to
colon cancer primary tumor samples classified as Duke's B stage with a positive survival
outcome (Duke's B survivor). These were selected from 59680 probesets on the
Affymetrix/Eos Hu03 GeneChip array such that the ratio of “average” colon cancer derived
liver metastases to “average” Duke's B survivor was greater than or equal to 3.0. The
“average” colon cancer derived liver metastases level was set to the 50th percentile. The
“average” Duke's B survivor level was set to the 50th percentile.

| | |
|---|---|
| Pkey: | Unique Eos probeset identifier number |
| ExAccn: | Exemplar Accession number, Genbank accession number |
| UnigeneID: | Unigene number |
| Unigene Title: | Unigene gene title |
| R1: | Genes up liver metastases vs Duke's B survivors |

| Pkey | ExAccn | UnigeneID | Unigene Title | R1 |
|---|---|---|---|---|
| 426101 | AL049987 | Hs.166361 | Homo sapiens mRNA; cDNA DKFZp564F112 (fr | 9.06 |
| 432572 | AI660840 | Hs.191202 | ESTs, Weakly similar to ALUE_HUMAN !!!! | 7.96 |
| 424878 | H57111 | Hs.221132 | ESTs | 7.88 |
| 428046 | AW812795 | Hs.155381 | ESTs, Moderately similar to I38022 hypot | 7.48 |
| 407284 | AI539227 | Hs.214039 | hypothetical protein FLJ23556 | 7.45 |
| 439943 | AW083789 | Hs.124620 | ESTs | 7.00 |
| 442369 | AI565071 | Hs.159983 | ESTs | 7.00 |
| 415116 | AA160383 | Hs.269956 | ESTs | 6.98 |
| 433517 | AW022133 | Hs.189838 | ESTs | 6.70 |
| 437176 | AW176909 | Hs.42346 | calcineurin-binding protein calsarcin-1 | 6.68 |
| 440524 | R71264 | Hs.16798 | ESTs | 6.62 |
| 408806 | AW847814 | Hs.289005 | Homo sapiens cDNA: FLJ21532 fis, clone C | 6.38 |
| 448974 | AL049390 | Hs.22689 | Homo sapiens mRNA; cDNA DKFZp586O1318 (f | 6.28 |
| 412088 | AI689496 | Hs.108932 | ESTs | 6.04 |
| 417670 | R07785 | | gb:yf15c06.r1 Soares fetal liver spleen | 5.95 |
| 440774 | AI420611 | Hs.127832 | ESTs | 5.91 |
| 426086 | T94907 | Hs.188572 | ESTs | 5.90 |
| 436100 | AA704806 | Hs.143842 | ESTs, Weakly similar to 2004399A chromos | 5.84 |
| 453204 | R10799 | Hs.191990 | ESTs | 5.84 |
| 407289 | AA135159 | Hs.203349 | Homo sapiens cDNA FLJ12149 fis, clone MA | 5.67 |
| 432435 | BE218886 | Hs.282070 | ESTs | 5.61 |
| 434963 | AW974957 | Hs.288719 | Homo sapiens cDNA FLJ12142 fis, clone MA | 5.60 |
| 421221 | AW276914 | Hs.326714 | Homo sapiens clone IMAGE:713177, mRNA se | 5.54 |
| 407328 | AA508857 | Hs.187748 | ESTs, Weakly similar to ALU1_HUMAN ALU S | 5.51 |
| 440980 | AL042005 | Hs.1117 | tripeptidyl peptidase II | 5.48 |
| 443651 | W22152 | Hs.282929 | ESTs | 5.42 |
| 412668 | AA456195 | Hs.10056 | hypothetical protein FLJ14621 | 5.29 |
| 444838 | AV651680 | Hs.208558 | ESTs | 5.24 |
| 433312 | AI241331 | Hs.131765 | ESTs, Moderately similar to I38937 DNA/R | 5.11 |
| 430665 | BE350122 | Hs.157367 | ESTs, Weakly similarto I78885 serine/th | 5.11 |
| 434966 | AA657494 | | gb:nt66f04.s1 NCI_CGAP_Pr3 Homo sapiens | 5.10 |
| 426897 | AW976570 | Hs.97387 | ESTs | 5.08 |
| 432954 | AI076345 | Hs.214199 | ESTs | 5.07 |
| 431416 | AA532718 | Hs.178604 | ESTs | 5.00 |
| 420717 | AA284447 | Hs.271887 | ESTs | 4.96 |
| 424950 | AA602917 | Hs.156974 | ESTs | 4.94 |
| 438962 | BE046594 | | gb:hn41c11.x1 NCI_CGAP_RDF2 Homo sapiens | 4.92 |
| 419999 | AI760942 | Hs.191754 | ESTs | 4.89 |
| 435812 | AA700439 | Hs.188490 | ESTs | 4.86 |
| 418662 | AI801098 | Hs.151500 | ESTs | 4.79 |
| 428065 | AI634046 | Hs.157313 | ESTs | 4.77 |
| 407618 | AW054922 | Hs.53478 | Homo sapiens cDNA FLJ12366 fis, clone MA | 4.75 |
| 435981 | H74319 | Hs.188620 | ESTs | 4.74 |
| 419145 | N99638 | | gb:za39g11.r1 Soares fetal liver spleen | 4.73 |
| 432340 | AA534222 | | gb:nj21d02.s1 NCI_CGAP_AA1 Homo sapiens | 4.72 |
| 447982 | H22953 | Hs.137551 | ESTs | 4.72 |
| 449509 | AA001615 | Hs.84561 | ESTs | 4.72 |
| 407946 | AA226495 | Hs.154292 | ESTs | 4.70 |
| 438607 | AW080237 | Hs.252884 | ESTs | 4.68 |
| 438406 | BE273296 | Hs.254467 | Homo sapiens cDNA FLJ13255 fis, clone OV | 4.62 |
| 426818 | AA554827 | Hs.289115 | DKFZp434A0131 protein | 4.62 |
| 452220 | BE158006 | Hs.212296 | ESTs | 4.60 |
| 436823 | AW749865 | Hs.293645 | ESTs, Weakly similar to I38022 hypotheti | 4.60 |
| 433854 | AA610649 | Hs.333239 | ESTs | 4.56 |
| 413816 | AW958181 | Hs.189998 | ESTs | 4.52 |
| 428079 | AA421020 | Hs.208919 | ESTs | 4.52 |
| 421097 | AI280112 | Hs.125232 | Homo sapiens cDNA FLJ13266 fis, clone OV | 4.50 |
| 417035 | AA192455 | Hs.22968 | pi Homo sapiens clone IMAGE:451939, mRNA se | 4.48 |
| 423974 | AL118754 | | gb:DKFZp761P1910_r1 761 (synonym: hamy2) | 4.44 |
| 449618 | AI076459 | Hs.15978 | KIAA1272 protein | 4.44 |
| 431615 | AW295859 | Hs.235860 | ESTs | 4.44 |
| 418876 | AA740616 | | gb:ny97f11.s1 NCI_CGAP_GCB1 Homo sapiens | 4.43 |
| 428279 | AA425310 | Hs.155766 | ESTs, Weakly similar to A47582 B-cell gr | 4.42 |
| 430573 | AA744550 | Hs.136345 | ESTs | 4.42 |
| 430929 | AA489166 | Hs.156933 | ESTs | 4.40 |
| 446099 | T93096 | Hs.17126 | hypothetical protein MGC15912 | 4.40 |
| 439362 | AI954880 | Hs.134604 | ESTs | 4.36 |
| 421999 | U50535 | Hs.110630 | Human BRCA2 region, mRNA sequence CG006 | 4.35 |
| 434220 | AI174777 | Hs.283039 | Homo sapiens PRO2492 mRNA, complete cds | 4.33 |
| 432925 | AA878324 | Hs.192734 | ESTs | 4.32 |
| 417819 | AI253112 | Hs.133540 | ESTs | 4.30 |
| 426848 | H72531 | Hs.36190 | ESTs | 4.30 |
| 429831 | AA564489 | Hs.137526 | ESTs | 4.30 |
| 433735 | AA608955 | Hs.109653 | ESTs | 4.30 |
| 418884 | AA230228 | Hs.59197 | ESTs | 4.28 |
| 413243 | AA769266 | Hs.193657 | ESTs | 4.26 |
| 431749 | AL049263 | Hs.306292 | Homo sapiens mRNA; cDNA DKFZp564F133 (fr | 4.23 |
| 428054 | AI948688 | Hs.266619 | ESTs | 4.22 |
| 413967 | AW204431 | Hs.117853 | ESTs, Weakly similar to I38022 hypotheti | 4.22 |
| 433230 | AW136134 | Hs.220277 | ESTs | 4.22 |
| 421057 | T58283 | Hs.10450 | Homo sapiens cDNA: FLJ22063 fis, clone H | 4.22 |
| 423578 | AW960454 | Hs.222830 | ESTs | 4.21 |
| 439717 | W94472 | Hs.59529 | ESTs, Moderately similar to ALU1_HUMAN A | 4.20 |
| 443696 | AW607444 | Hs.134622 | ESTs | 4.20 |
| 432722 | AA830532 | Hs.326160 | ESTs | 4.18 |
| 435756 | AI418466 | Hs.33665 | ESTs | 4.14 |
| 428825 | AI084336 | Hs.128783 | ESTs, Weakly similar to I38022 hypotheti | 4.14 |
| 439451 | AF086270 | Hs.278554 | heterochromatin-like protein 1 | 4.12 |
| 445943 | AW898533 | Hs.181574 | ESTs | 4.12 |
| 450219 | AI826999 | Hs.224624 | ESTs | 4.12 |
| 431379 | AA504264 | Hs.182937 | peptidylprolyl isomerase A (cyclophilin | 4.11 |
| 432451 | AW972771 | Hs.292471 | ESTs, Weakly similar to ALU1_HUMAN ALU S | 4.10 |
| 443148 | AI034357 | Hs.211194 | ESTs, Weakly similar to ALU8_HUMAN ALU S | 4.08 |
| 450177 | AI698091 | Hs.107845 | ESTs | 4.08 |
| 420911 | U77413 | Hs.100293 | O-linked N-acetylglucosamine (GlcNAc) tr | 4.06 |
| 421114 | AW975051 | Hs.293156 | ESTs, Weakly similar to I78885 serine/th | 4.06 |
| 432731 | R31178 | Hs.287820 | fibronectin 1 | 4.06 |
| 433588 | AI056872 | Hs.133386 | ESTs | 4.06 |
| 434658 | AI624436 | Hs.310286 | ESTs | 4.06 |
| 444040 | AF204231 | Hs.182982 | golgin-67 | 4.06 |
| 429512 | AA453987 | Hs.144802 | ESTs | 4.06 |
| 443349 | AI052572 | Hs.269864 | ESTs, Weakly similar to ALU1_HUMAN ALU S | 4.04 |
| 439867 | AA847510 | Hs.161292 | ESTs | 4.04 |
| 425955 | T96509 | Hs.248549 | ESTs, Moderately similar to S65657 alpha | 4.02 |
| 431393 | AW971493 | Hs.134269 | ESTs, Highly similar to cytokine recepto | 4.00 |
| 432125 | AW972667 | Hs.287510 | Homo sapiens cDNA FLJ12300 fis, clone MA | 4.00 |
| 435468 | AW362803 | Hs.166271 | ESTs | 3.97 |
| 412059 | AA317962 | Hs.249721 | ESTs, Moderately similar to PC4259 ferri | 3.95 |
| 446682 | AW205632 | Hs.211198 | ESTs | 3.95 |
| 441328 | AI982794 | Hs.159473 | ESTs | 3.92 |
| 455778 | BE088746 | | gb:CM2-BT0693-210300-123-d09 BT0693 Homo | 3.90 |
| 438996 | AW748336 | Hs.168052 | KIAA0421 protein | 3.86 |
| 418303 | AA215701 | Hs.186541 | ESTs, Weakly similar to I38022 hypotheti | 3.85 |
| 444816 | Z48633 | Hs.283742 | H.sapiens mRNA for retrotransposon | 3.84 |
| 429355 | AW973253 | Hs.292689 | ESTs | 3.83 |
| 438578 | AA811244 | Hs.164168 | ESTs | 3.83 |
| 432945 | AL043683 | Hs.8173 | hypothetical protein FLJ10803 | 3.83 |
| 435318 | T97301 | Hs.18026 | ESTs | 3.82 |
| 449941 | AW450536 | Hs.209260 | ESTs | 3.80 |
| 424915 | R42755 | Hs.23096 | ESTs | 3.76 |
| 449987 | AW079749 | Hs.184719 | ESTs, Weakly similar to ALU1_HUMAN ALU S | 3.76 |
| 416265 | AA177088 | Hs.190065 | ESTs | 3.75 |
| 413497 | BE177661 | | gb:RC1-HT0598-020300-011-h02 HT0598 Homo | 3.74 |
| 412093 | BE242691 | Hs.14947 | ESTs | 3.74 |
| 413822 | R08950 | Hs.272044 | ESTs, Weakly similar to ALU1_HUMAN ALU S | 3.73 |
| 431915 | AK000777 | Hs.272197 | Homo sapiens cDNA FLJ20770 fis, clone CO | 3.68 |
| 434442 | AA737415 | Hs.152826 | ESTs | 3.63 |
| 434959 | AW974949 | Hs.186564 | ESTs, Weakly similar to I38022 hypotheti | 3.63 |
| 427704 | AW971063 | Hs.292882 | ESTs | 3.62 |
| 426510 | AW861225 | Hs.194637 | BANP homolog, SMAR1 homolog | 3.60 |
| 435714 | AA699325 | Hs.269880 | ESTs | 3.60 |
| 432598 | AI341227 | Hs.157106 | ESTs | 3.57 |
| 438543 | AA810141 | Hs.192182 | ESTs | 3.55 |
| 422068 | AI807519 | Hs.104520 | Homo sapiens cDNA FLJ13694 fis, clone PL | 3.54 |
| 418259 | AA215404 | Hs.137289 | ESTs | 3.54 |
| 428290 | AI932995 | Hs.183475 | Homo sapiens clone 25061 mRNA sequence | 3.49 |
| 419457 | AA243146 | Hs.209334 | ESTs, Moderately similar to S23A_HUMAN P | 3.47 |
| 439312 | AA833902 | Hs.270745 | ESTs | 3.47 |
| 408784 | AW971350 | Hs.63388 | ESTs | 3.45 |
| 456332 | AA228357 | | gb:nc39d05.r1 NCI_CGAP_Pr2 Homo sapiens | 3.45 |
| 424762 | AL119442 | Hs.183684 | eukaryotic translation initiation factor | 3.44 |
| 442884 | AI076570 | Hs.134053 | ESTs | 3.44 |
| 421023 | AW449855 | Hs.96557 | Homo sapiens cDNA FLJ12727 fis, clone NT | 3.43 |
| 434575 | AI133446 | Hs.299964 | Homo sapiens clone FLB7723 PRO2055 mRNA, | 3.42 |
| 430433 | AA478883 | Hs.273766 | ESTs | 3.39 |
| 419317 | AA236282 | Hs.172318 | ESTs | 3.38 |
| 448710 | T62926 | Hs.304184 | ESTs | 3.37 |
| 439322 | H72245 | Hs.188635 | ESTs | 3.37 |
| 430332 | R51790 | Hs.239483 | Human clone 23933 mRNA sequence | 3.35 |
| 411755 | BE327036 | Hs.117494 | ESTs | 3.33 |
| 427882 | AA640987 | Hs.193767 | ESTs | 3.28 |
| 438899 | AF085833 | Hs.135624 | ESTs | 3.28 |
| 436535 | AW295687 | Hs.254420 | ESTs | 3.25 |
| 434936 | AI285970 | Hs.183817 | ESTs | 3.22 |
| 451730 | AF095887 | Hs.26937 | brain and nasopharyngeal carcinoma susce | 3.18 |
| 447514 | AI809314 | Hs.208501 | ESTs, Weakly similar to B34087 hypotheti | 3.18 |
| 413672 | BE156536 | | gb:QV0-HT0368-310100-091-h10 HT0368 Homo | 3.16 |
| 435073 | AA664078 | | gb:ac04a05.s1 Stratagene lung (937210) H | 3.13 |
| 450295 | AI766732 | Hs.210628 | ESTs | 3.13 |
| 419341 | N71463 | Hs.118888 | ESTs, Weakly similar to ALU1_HUMAN ALU S | 3.13 |
| 434495 | AW352170 | Hs.129086 | Homo sapiens cDNA FLJ12007 fis, clone HE | 3.12 |
| 408113 | T82427 | Hs.194101 | Homo sapiens cDNA: FLJ20869 fis, clone A | 3.12 |
| 456437 | AI924228 | Hs.115185 | ESTs, Moderately similarto PC4259 ferri | 3.12 |
| 421489 | AI922821 | Hs.32433 | ESTs | 3.12 |
| 436090 | AI640635 | Hs.116468 | EST | 3.11 |
| 450230 | AW016607 | Hs.201582 | ESTs | 3.11 |
| 436011 | BE466173 | Hs.145696 | splicing factor (CC1.3) | 3.09 |
| 418720 | AI381687 | Hs.39526 | ESTs | 3.09 |
| 433102 | AI343966 | Hs.158528 | ESTs | 3.08 |
| 436150 | AW510927 | Hs.125243 | ESTs | 3.05 |
| 440116 | AI798851 | Hs.283108 | hemoglobin, gamma G | 3.04 |
| 414900 | AW452420 | Hs.245678 | ESTs | 3.04 |
| 435937 | AA830893 | Hs.119769 | ESTs | 3.02 |
| 424848 | AI263231 | Hs.327090 | EST | 3.02 |
| 435354 | AA678267 | Hs.117115 | ESTs | 3.00 |

TABLE 23A
Table 23A show the accession numbers for those pkeys lacking
unigeneID's for tables 1-20A, 21A, 22A, and 23A. For
each probeset we have listed the gene cluster number from
which the oligonucleotides were designed. Gene clusters were
compiled using sequences derived from Genbank ESTs and mRNAs.
These sequences were clustered based on sequence similarity
using Clustering and Alignment Tools (DoubleTwist, Oakland
California). The Genbank accession numbers for sequences
comprising each cluster are listed in the “Accession”
column.

| | |
|---|---|
| Pkey: | Unique Eos probeset identifier number |
| CAT number: | Gene cluster number |
| Accession: | Genbank accession numbers |

| Pkey | CAT Number | Accession |
|---|---|---|
| 413497 | 1373771_1 | BE177661 H06215 BE144709 BE144829 |
| 413672 | 1382512_1 | BE156536 BE156439 BE156700 BE156449 |
| | | BE156653 BE156533 BE156524 BE156670 |
| | | BE156721 BE156723 |
| 417670 | 1692163_1 | R07785 T85948 T86972 |
| 418876 | 179960_1 | AA740616 AA654854 AA229923 |
| 419145 | 182217_1 | N99638 AW973750 AA328271 H90994 |
| | | AA558020 AA234435 N59599 R94815 |
| 423974 | 233842_1 | AL118754 AA333202 H38001 |
| 432340 | 345248_1 | AA534222 AA632632 T81234 |
| 434966 | 396504_1 | AA657494 AI582663 AI581639 |
| 435073 | 399701_1 | AA664078 AW363313 AA805009 |
| 438962 | 467390_1 | BE046594 BE046667 AA828585 AI207343 |
| 455778 | 1364506_1 | BE088746 BE088802 BE088755 BE088876 |
| | | BE088947 BE088881 BE088952 |
| 456332 | 179104_1 | AA228357 AW841786 AW841716 |

TABLE 24
34 GENES DOWN-REGULATED IN COLON CANCER
DERIVED LIVER METASTASES COMPARED TO COLON CANCER
PRIMARY TUMOR SAMPLES CLASSIFIED AS DUKE'S B SURVIVOR
Table 24 shows 34 genes down-regulated in colon cancer derived liver metastases compared
to colon cancer primary tumor samples classified as Duke's B stage with a positive survival
outcome (Duke's B survivor). These were selected from 59680 probesets on the
Affymetrix/Eos Hu03 GeneChip array such that the ratio of “average” colon cancer derived
liver metastases to “average” Duke's B survivor was greater than or equal to 0.25. The
“average” colon cancer derived liver metastases level was set to the 50th percentile. The
“average” Duke's B survivor level was set to the 50th percentile.

| | |
|---|---|
| Pkey: | Unique Eos probeset identifier number |
| ExAccn: | Exemplar Accession number, Genbank accession number |
| UnigeneID: | Unigene number |
| Unigene Title: | Unigene gene title |
| R1: | Genes down liver metastases vs Duke's B survivors |

| Pkey | ExAccn | UnigeneID | Unigene Title | R1 |
|---|---|---|---|---|
| 414522 | AW518944 | Hs.76325 | step II splicing factor SLU7 | 0.05 |
| 416768 | AA363733 | Hs.1032 | regenerating islet-derived 1 alpha (panc | 0.07 |
| 409153 | W03754 | Hs.50813 | hypothetical protein FLJ20022 | 0.07 |
| 414555 | N98569 | Hs.76422 | phospholipase A2, group IIA (platelets, | 0.11 |
| 418007 | M13509 | Hs.83169 | matrix metalloproteinase 1 (interstitial | 0.11 |
| 424326 | NM_014479 | Hs.145296 | disintegrin protease | 0.11 |
| 428934 | AF039401 | Hs.194659 | chloride channel, calcium activated, fam | 0.12 |
| 417233 | W25005 | Hs.24395 | small inducible cytokine subfamily B (Cy | 0.12 |
| 422260 | AA315993 | Hs.105484 | regenerating gene type IV | 0.12 |
| 425196 | AL037915 | Hs.155097 | carbonic anhydrase II | 0.13 |
| 433336 | AF017986 | Hs.31386 | secreted frizzled-related protein 2 | 0.13 |
| 450685 | L15533 | Hs.423 | pancreatitis-associated protein | 0.14 |
| 407811 | AW190902 | Hs.40098 | cysteine knot superfamily 1, BMP antagon | 0.15 |
| 414798 | AI286323 | Hs.97411 | hypothetical protein MGC12335 | 0.16 |
| 452852 | AK001972 | Hs.30822 | hypothetical protein FLJ11110 | 0.17 |
| 447513 | AW955776 | Hs.313500 | ESTs, Moderately similar to ALU_HUMAN A | 0.17 |
| 423541 | AA296922 | Hs.129778 | gastrointestinal peptide | 0.17 |
| 425071 | NM_013989 | Hs.154424 | deiodinase, iodothyronine, type II | 0.18 |
| 406636 | L12064 | | gb:Homo sapiens (clone WR4.12VL) anti-th | 0.18 |
| 421515 | Y11339 | Hs.105352 | GalNAc alpha-2, 6-sialyltransferase 1,1 | 0.18 |
| 428368 | BE440042 | Hs.83326 | matrix metalloproteinase 3 (stromelysin | 0.19 |
| 414812 | X72755 | Hs.77367 | monokine induced by gamma interferon | 0.20 |
| 452594 | AU076405 | Hs.29961 | solute carrier family 26 (sulfate transp | 0.20 |
| 428227 | AA321649 | Hs.2248 | small inducible cytokine subfamily B (cy | 0.21 |
| 408741 | M73720 | Hs.646 | carboxypeptidase A3 (mast cell) | 0.21 |
| 453064 | R40334 | Hs.89463 | potassium large conductance calcium-acti | 0.21 |
| 431727 | AW293464 | Hs.162031 | ESTs | 0.22 |
| 433658 | L03678 | Hs.156110 | immunoglobulin kappa constant | 0.22 |
| 442064 | AI422867 | Hs.88594 | ESTs | 0.22 |
| 417880 | BE241595 | Hs.52848 | selectin L (lymphocyte adhesion molecule | 0.22 |
| 430280 | AA361258 | Hs.237868 | interteukin 7 receptor | 0.23 |
| 452877 | AI250789 | Hs.32478 | ESTs | 0.23 |
| 410310 | J02931 | Hs.62192 | coagulation factor III (thromboplastin, | 0.24 |
| 402408 | | | | 0.24 |

TABLE 24B

| | |
|---|---|
| Pkey: | Unique number corresponding to an Eos probeset |
| Ref: | Sequence source. The 7 digit numbers in this |
| | column are Genbank Identifier (GI) numbers. |
| | “Dunham I. et al.” refers to the |
| | publication entitied “The DNA sequence of |
| | human chromosome 22.” Dunham I. et al., |
| | Nature (1999) 402:489-495. |
| Strand: | Indicates DNA strand from which exons were |
| | predicted. |
| Nt_position: | Indicates nucleotide positions of predicted exons. |

| Pkey | Ref | Strand | Nt_position |
|---|---|---|---|
| 402408 | 9796239 | Minus | 110326-110491 |

TABLE 25
Table 25 depicts Seq ID No., UnigeneID, UnigeneTitle, Pkey, and ExAccn for all of the
sequences in Table 26. Seq ID No links the nucleic acid and protein sequence
information in Table 26 to Table 25.

| | |
|---|---|
| Pkey: | Unique Eos probeset identifier number |
| ExAccn: | Exemplar Accession number, |
| | Genbank accession number |
| UnigeneID: | Unigene number |
| Unigene Title: | Unigene gene titie |
| Seq.ID.No.: | Sequence Identification Number |
| | found in Table 26 |

| Pkey | ExAccn | UnigeneID | Unigene Title | Seq ID No. |
|---|---|---|---|---|
| 426101 | AL049987 | | Homo sapiens mRNA; cDNA DKFZp564F112 (fr | 1-4 |
| 419145 | N99638 | | gb | 5 & 6 |
| 426818 | AA554827 | Hs.340046 | DKFZp434A0131 protein | 7 & 8 |
| 421057 | T58283 | | Homo sapiens cDNA | 9 |
| 446619 | AU076643 | Hs.313 | secreted phosphoprotein 1 (osteopontin, | 10 & 11 |
| 431958 | X63629 | Hs.2877 | cadherin 3, type 1, P-cadherin (placenta | 12 & 13 |
| 409041 | AB033025 | Hs.50081 | Hypothetical protein, XP_051860 (KIAA119 | 14 & 15 |
| 443162 | T49951 | Hs.9029 | DKFZP434G032 protein | 16 & 17 |
| 436385 | BE551618 | Hs.144097 | ESTs | 18-20 |
| 447033 | AI357412 | Hs.157601 | ESTs | 21 & 22 |
| 439608 | AW864696 | Hs.301732 | hypothetical protein MGC5306 | 23-27 |
| 449032 | AA045573 | Hs.22900 | nuclear factor (erythroid-derived 2)-lik | 28 & 29 |
| 442577 | AA292998 | Hs.163900 | ESTs | 30 & 31 |
| 429970 | AK000072 | Hs.227059 | chloride channel, calcium activated, fam | 32 & 33 |
| 424566 | M16801 | Hs.1790 | nuclear receptor subfamily 3, group C, m | 34 & 35 |
| 457407 | AA505035 | Hs.345911 | ESTs | 36 |
| 430378 | Z29572 | Hs.2556 | tumor necrosis factor receptor superfami | 37 & 38 |
| 417332 | AW972717 | Hs.288462 | hypothetical protein FLJ21511 | 39 & 40 |

TABLE 25A

| | |
|---|---|
| Pkey: | Unique Eos probeset identifier number |
| CAT number | Gene cluster number |
| Accession: | Genbank accession numbers |

| Pkey | CAT Number | Accession |
|---|---|---|
| 409041 | | 10962_2 AB033025 AL359061 AL045836 AI751521 AI752804 AI752650 AA853580 AI752290 AA853460 AI752769 |
| | | AA852309 AA853785 AA853219 AW068503 AI752069 AL049389 AW068368 6E439518 W52813 BE141833 AI940574 |
| | | AI750606 AL109718 AA242845 AA315795 AA307741 AW954603 AI752070 AA350794 AI752649 AA307755 |
| | | AW951677 AA298896 BE439692 AA852453 AW068826 AW853984 AA418236 AA639417 AW290917 AI750592 |
| | | AI752768 AL045837 AI926513 AW262903 BE439819 AI459360 AW339074 AW295181 AW029483 AI750945 |
| | | AI750659 AI752525 AI147688 BE440122 AI751522 AI473816 AI752291 AI694639 AI925816 AA599476 |
| | | AA242752 AW021892 AI755098 AW469299 |
| | | AW769363 AA853579 AI784082 AA852454 AI925501 AA976657 AW150473 AW166734 |
| 417332 | 166755_1 | AW972717 AA523805 AI962905 AI373245 AW235545 AI812045 AW589434 AI826824 AW572339 |
| | | AI377551 AA195718 AI868470 |
| 419145 | 182217_1 | N99638 AW973750 AA328271 H90994 AA558020 AA234435 N59599 R94815 |
| 421057 | 198849_1 | T58283 AA765038 AA283052 H99396 AA814751 AI032674 N81016 N81017 BE222349 AA830545 |
| 424566 | 2408_1 | M16801 NM_000901 D57171 AL041328 AF068623 AI201179 AA151766 AA568349 |
| | | AI698649 AI692765 BE327401 AA744953 |
| | | AA744951 AW361986 AV651840 T29894 AW945145 AW945145 W24096 AI183952 AI458972 |
| | | AW190993 AI765359 AI634663 AI741201 AW418944 AI767551 AA679687 AW772342 |
| | | AW629508 BE504300 AI251790 AI522294 AA724341 AW615402 |
| | | AI537570 AA470665 AI458375 AW768901 AA447079 T23537 AI783744 R44301 D56621 |
| | | N91919 AA149749 |
| 426101 | 26088_1 | AL049987 AW362842 T78981 AA247541 AI217018 AW961515 AA632986 AA663108 BE326465 |
| | | AW872412 AI024889 AA453725 |
| | | BE150458 AA229448 AA442638 AA442648 AI916737 AA480220 AA868553 AI827987 AI005467 R31132 AI742087 |
| | | AA442379 N56349 AW769479 AI860142 AI917507 AA813604 AI860141 AI459289 AA522837 AI354470 AI921333 |
| | | BE466760 AW971193 AW103830 AW277065 AW020895 AI187977 N28268 AI084517 R95914 AA833517 AA563934 |
| | | AA437299 AA436880 AA447794 AA812876 AA663178 R31089 AI472712 R64648 AA600372 AA229164 AA703066 |
| | | AW270324 AI191725 AA551512 AA493776 |
| 426818 | 272427_1 | AA554827 AA701001 AW972954 AL039129 AA385540 AA911663 |
| 429970 | 31134_1 | AK000072 AW840683 AW843764 AW844444 AW844515 AW603469 AW862395 AI860838 AW511708 AF127035 |
| | | NM_012128 AK000138 |
| 430378 | 3170_1 | Z29572 AW976377 AA286871 AA633372 AA987627 AA743176 AI865358 AJ006884 AF031845 Z14955 |
| 431958 | 3394_1 | X63629 NM—001793 BE175433 BE153414 BE153425 AW364593 BE315317 AW950190 AA314252 |
| | | BE142943 AW365220 AW368405 BE004269 AW366568 AL040609 AI829273 AI591168 BE146183 AI631060 |
| | | AI830793 W78081 W92295 AI927422 BE009313 AI371793 AW993031 AI204659 AA535113 AW993030 |
| | | AI190281 AA555159 AW269637 AW993146 AI149268 |
| | | AA425217 AW473194 AI890930 AA551993 AI952106 W92308 AI827275 W45400 AI952328 AW609233 |
| | | AA774611 AA551779 AI913967 AI798658 AI537658 AW517535 AA632236 AW339148 AW589522 |
| | | AA836945 AA961263 AW015821 AW272946 C00249 W40333 BE143121 |
| 436385 | 418907_1 | BE551618 AI207338 BE220568 AI261568 AW841737 AA714722 AA946891 AI033239 |
| 439608 | 47438_3 | AW864696 AW338889 AI342866 AA084522 AI244150 AI610339 AA425635 AA764930 AA976965 |
| | | AW805766 AA057765 AW805845 AW802595 AA262971 AI969620 N75323 BE549060 AW805725 AA025809 |
| | | N80776 N64595 AW073372 AA025493 AI819475 AW028879 AW189496 AA442907 AW410368 AI911629 |
| | | N71276 AW316922 AW805838 AA043880 AW189184 AA449758 AA748153 AA705608 AI910643 AA279492 |
| | | BE160119 AW805761 AA026262 AA782207 AW057652 AW805768 |
| | | H21998 AW194254 AW275178 AA449040 AA279582 N76314 N54348 |
| 442577 | 54549_1 | AA292998 AW238350 AI676059 AW074092 BE566458 AW078677 AW514801 AW073701 AW170620 |
| | | AI523736 AI580870 AI923975 AI393326 AI700229 AW450814 AW628452 AI671457 AA937534 |
| | | AI889894 AW339423 AW291875 AA551874 AI682314 AI926227 AA397375 |
| 443162 | 5613_1 | T49951 AA025326 H04839 AA393303 R63101 W57657 W25628 AI961431 R71165 N39940 |
| | | H01548 H01759 AA641624 AI634930 AA595296 AW994770 AW994747 BE047247 W38159 AA858133 |
| | | AI701944 AW386273 AA676625 R24676 R79410 AA922863 AI151319 H01013 AA024482 W02674 H01456 |
| | | AI150858 AW135972 AW631167 AI270332 H04750 T49622 AA004543 |
| | | R63061 AI093066 AI247539 H01225 H03388 AW472933 AA382448 AI219287 N27194 AW389613 |
| | | AA649738 AW994764 AW389614 R25176 AA897262 R71626 AA909471 R71240 AW811917 R76109 |
| | | AI202312 AI858010 R76162 AL117538 R79411 T58658 AW994674 |
| 446619 | 685_1 | AU076643 AA594604 AA348866 R18197 AA345192 AA337773 AA089791 R84435 AA337838 AW392167 |
| | | AA075190 D55416 AW150380 AW366257 AA579816 H93048 AW385889 AW385697 AI186216 AW581197 |
| | | AL037509 AB019562 AA232626 R97905 AW368019 AA242891 AW888502 AI798331 AW385835 |
| | | AW581221 T96947 H87989 AA369511 AA075191 R80742 AA366406 |
| | | W92752 H45588 AI864016 AW888497 BE004992 AI384110 AI624258 AI627593 W92728 AI682719 |
| | | AA948208 AA171734 N40517 J04765 AA379957 AA362403 NM_000582 AF052124 AA300290 |
| | | AA333447 AA343721 AW889543 BE586767 R76601 R18015 AA100531 AA489963 AA101296 AA363513 |
| | | AA344088 AA336750 T77505 D56440 AL110351 AL110331 F12195 R20175 AA336664 H17766 AA363538 |
| | | AA363590 D28760 AW578517 AA383531 AI814667 AA846899 AA368253 AW951285 AA297992 |
| | | AA327756 AW361609 AW815455 AW815427 AW815428 D54182 AW852200 AA171630 W27018 AW815864 |
| | | AW379995 AW378222 AW362610 BE566022 AW021023 C17352 D58435 AA345409 AI623991 AW020967 |
| | | AI924770 AI799443 AW946393 AA991239 AI571617 AI935181 AI923999 AI826895 AI860319 |
| | | AW189873 AW270353 AW023584 AI813811 R99929 AW339056 AA913152 AI636352 AI829394 AW151077 |
| | | AW192580 AI570119 AI086391 AW021764 AW519154 AI375193 AW268678 |
| | | BE465690 AW019983 AW268654 AI573138 AI141809 AI954553 AI559242 AA568945 AA886417 |
| | | AW338527 AI635881 BE465666 AI921239 AA968537 AI958027 AA911981 AI827661 AW511046 |
| | | BE619780 AI922227 AI811870 AW190131 AW129220 AW512906 AI290757 AI819088 AI623771 |
| | | AA775616 BE349419 AI126375 H88773 AI241758 AW275157 AI337848 |
| | | AI613425 AI631387 AA922631 AI273483 AI982898 AW168957 AI446481 BE501588 BE048264 |
| | | AI499922 AW023812 BE220523 AW973846 BE349276 AI141091 AA976060 AW973845 AA101270 |
| | | AI582472 AW613675 AI139360 AI282627 AI276044 N22345 |
| | | AI261875 AA634136 AI824468 AW887693 N27107 R21504 AI042223 N22067 AW196871 |
| | | AI581019 BE004973 AA252035 N22087 AA570717 H11250 AI804026 AA368098 AA021512 |
| | | H08842 N26275 AA176368 AI758758 AA570371 AA232574 |
| | | BE221177 AW190221 AW471386 AA78225 AI422140 AI624521 AA719775 AA300291 AA568657 |
| | | AI871430 BE465630 N71862 T72587 W92721 H88774 D64383 AW103693 AW089986 AI382689 |
| | | R42363 R44962 T98770 AA357374 AW022074 AI356207 |
| | | T29241 AW089431 AI933875 N66267 N67352 AA121786 AA363910 F09824 T95818 N66888 |
| | | R80550 AI280887 AW196719 R59299 AW021049 H73469 AI954311 BE439454 AW079450 |
| | | AW973850 AA348338 AW896006 AW268145 AA853631 H17650 |
| | | R39537 N66873 N67240 H06298 AI784199 R44260 AA904118 AA911756 F04544 AA807809 |
| | | AA665210 AI696448 T29719 AA837240 T64844 H08926 |
| 447033 | 704603_1 | AI357412 AI870708 AI590539 W07459 |
| 449032 | 7945_1 | AA045573 AA279920 R20139 AA372783 AW963629 H21473 R78318 W74359 AA022505 |
| | | AA369091 AW084075 AA503638 AV660815 AI216262 AA779843 BE219825 AF125534 |
| | | AW972129 AI919099 AI621283 AI300590 AI953701 AA331415 AW610546 |
| | | AW793050 AI953679 AW793047 AW610543 AI671103 AW292105 AW024112 R77947 |
| | | W76339 AA305111 AA132523 AA227467 H21401 AW366572 AW024129 AI701886 AI654744 |
| | | BE042803 AI347173 AW866053 AW662710 R36639 AI469777 AA962733 |
| | | AI865368 AA501998 AW866054 BE178974 |
| 457407 | 333252_1 | AA505035 AW235098 AI634028 |

TABLE 26

| | |
|---|---|
| Seq ID NO: 1 DNA sequence | |
| Nucleic Acid Accession #: see Table 25 & 25A for complete list | |
| 1 11 21 31 41 51 | |
| \| \| \| \| \| \| | |
| CAATATAGTA CAATAACTAT TTGCATGACA TTTACATCGG ATATTATGAG TGATCTAGAG | 60 |
| TTGATATGAA GTATATGGGA GGATGTGCAA AGGTGATGTG CAAATACTAT GTCATTTTAT | 120 |
| AGGGGGGACT TGAGTATCCT TTGTTACCCT CAGGAGATCC TGAAACCAGT CCCCCATGGA | 180 |
| TACTGAGGGC TGACTGTATA GTCCTATCCT CACGGAACTT TCATTCTAAT GGGGGAAGAC | 240 |
| TGACTATAAA CAAAATATAT GTAATAGGTG GTGGTAAGTA CCGTGGAGAA GTAACAAATG | 300 |
| GGGCAAAGTG AGTTATACAG CTCCATTCTT AGAAACCTTG GAGTACTTTT CTTAGTTTAT | 360 |
| ACTCGTGGTG GTTTCCTTTT GTCTCCTTTA TTACATGGGA CTCTGACATG TGCCCATAGC | 420 |
| TAGGGTGACA GTAGGATCTA CCCGATAGTA GGGTGGCAGT AGGATCTACC CAAAAAGCGT | 480 |
| CCTGCTGATA CAGGACCAAA GCATCCTGTT GTTCTCGAGC CTATAAAAAG AGCTAATGGT | 540 |
| GTTGCTTCTC TTAACTGTGG CCTCCTACAC TGTGTTTTGG ATGATTGGTG ATGTCTTGGA | 600 |
| TATTCTGTTT CTTTGGAACT TTGAATATAC AACACTTTAC TAGGGAATTA GCAATGGAAG | 660 |
| CAGAGCAAAG ATGTACAGAG GAAACAATGC GTAACTCTGA TGGAATTGAA GTCATGAGGC | 720 |
| AGCAGAGAGC TTAAATTACA GCTTTAAAAA TTTTTATTTT TTAGAGGGAA TTTACTTGGG | 780 |
| AGTAACAGCA GTAATAGTTA ACGGAGCCAG AATGCTTGAG TCATATAATT GCAAAGCAGA | 840 |
| GTTGGGAGCA ACAGATGCTA AAGAGTAGTT GCTGTAGTTC CTCTTTGGGT CGTAGGAGCA | 900 |
| GTTGTCATAT TACTATATAG CTACTGCATG AAGAAGAGTT CTTAGTGAGG CCTGGGTGAA | 960 |
| CAGCTCTTCT TAGTATTCTG TGTGACCCCA TTTGACCTTT TAACAAATCC CTAAGTAAAT | 1020 |
| AAATAGCCCC TCAGGAAAAC TAAGTTTTTC TCTGCTGTTT TTTTGCTTGA GAGAGCTATA | 1080 |
| ACTGTAATAG ACTTATATTT CTGAACATTT TAGTGCTTGC CAATATTTGG TAATATTTAT | 1140 |
| GTTTCCTATA TTTGTAATGA ACATTCTTCT TCCGGTACAT TTTTTGTTAA ATTATTGTTT | 1200 |
| GATGGATAAA AGTTCACCTT TTATTGTATA AAATTGACTG AGATTAATTT ATACACATTG | 1260 |
| ACAATGGGTA AATAGAATTT TTCAGATTAT TAAAAGCTGA AGGATGACCA CGTAAGCAAA | 1320 |
| AAAAAAAAAA AAAAAACCAA CAAAAATAAA CCCAAACCCC TCAAACAATT TCGAACACGA | 1380 |
| AACATTCTTC TGATGCCGGC ATCCCTGCTT GCAGGTGTGA AGGGGGCAGG AATCAGCGAG | 1440 |
| GTGTCCTGGG CTGAGTCCCC GGGGAAGAAT ATGAT | |
| Seq ID NO: 2 DNA sequence | |
| Nucleic Acid Accession #: X83301.1 | |
| 1 11 21 31 41 51 | |
| \| \| \| \| \| \| | |
| GCAAAGCCAG CTGGGCTCCT GAGTCCGGTG GGTACTTGGA GAACTTACTA CGTCTAGCTG | 60 |
| GAGGATTGTA AATGCACCAA TCAGCATGCT GTGTCTAGCT CAAGATTTTC TCCATCCCCT | 120 |
| TATTTTGGGC CAGTGGCTGT CATTACATAT GAGATGAGTC TCTTGAAGAC TACAGATGAA | 180 |
| CTCAAGCTCC ATGAGGAGAT GTTTCATTGT CGAGAGCAGT CATGATGGCC TGCACTCCAC | 240 |
| ACAATGCAAC AGAGTGAAAG AGCAGGTTCT GCTTCTTTGG TGTAGTCCTG AAGCTTCCTA | 300 |
| AGAAACTTCA CATCAGGTGA TGGATAGGAG CAACCCTGTA AAACCAGCCT TAGACTATTT | 360 |
| TTCAAACAGG CTGGTGAATT ACCAGATCTC CGTCAAGTGC AGTAACCAGT TCAAGTTGGA | 420 |
| AGTGTGTCTT TTGAATGCAG AGAACAAAGT CGTGGACAAC CAGGCTGGGA CCCAGGGCCA | 480 |
| GCTGAAGGTG CTGGGTGCCA ACCTCTGGTG GCCGTACCTG ATGCACGAAC ACCCCGCCTA | 540 |
| CCTGTACTCC TGGGAGGATG GTGATTGCTC ACACCAAAGC CTTGGACCCC TCCCAGCCTG | 600 |
| TGACCTTTGG GACCAACTCC ACCTACGCAG CAGACAAGGG GGCTCTGTAT GTGGATGTGA | 660 |
| TCCGTGTGAA CAGCTACTAC TCTTGGTATC GCAACTACGG GCACCTGGAG TTGATTCGGC | 720 |
| TGCAGCTGGC CGCCCAGTTT GAGAATTGGT GTGAGACATC ACAATCCCAT TATTCAGAGC | 780 |
| GCGTATGGAG TGGAAACGCT TGTAGGGTTT CACCAGGGCT GGTGAATTAC CAGATCTCCG | 840 |
| TCAAGTGCAG TAACCAGTTC AAGTTGGAAG TATGTCTTTT GAATGCAGAA AACAAAGTCG | 900 |
| TGGACAACCA GGCTGGGACC CAGGGCCAGC TGAAGGTGCT GGTGCCAACC TCTGGTGGCC | 960 |
| GTACCTGATG CACGAACACC CCGCCTACCT GTACTCGTGG GAGGATGGTG ATTGCTCACA | 1020 |
| CCAAAGCCTT GGACCCCTCC CAGCCTGTGA CCTTTGGGAC CAACTCCACC TACGCAGCAG | 1080 |
| ACAAGGGGGC TCTGTATGTG GATGTGATCC GTGTGAACAG CTACTACTCT TGGTATCGCA | 1140 |
| ACTACGGGCA CCTGGAGTTG ATTCGGCTGC AGGCCCTGCA GCTGGCCGCC CAGTTTGTGA | 1200 |
| ATTGGTGTAA GACATCACAA TCCCATTATT CAGAGCGCGT ATGGAGTGGA AACGCTTGTA | 1260 |
| GGGTTTCACC AGTCTTTCCC AGGGAACTCC GATGAAGTGT TCCAACAAAA TGAGCGAGTG | 1320 |
| AACCAAGAAG AGGATGACAT TAGATCCAGG AGATACAACA GAGGAGATAA TCTCCAGGAT | 1380 |
| GCCTGTGAAG AAAGATCCCT GGATCCCAGG ATGATTATAG GACAAGTTGT TCATAATCCA | 1440 |
| GCAGGCCAGA AGACTTCCAG GGAAACTCAT TTCAAGATGA AAATGGACCA GCCGCAGTGG | 1500 |
| CTCACGCCTG TAATACCAGC ACTTTGGGAG GCTGAGGCGG GCGGATCACT TGAGGTCAAG | 1560 |
| AGTTTGAAAC TAGCCTGGCC AACGTGGCAA AACTCCATCT CTATTAAAGA TACAAAAATT | 1620 |
| AGCCAGGCAT AGTGGTGCAT GCCTGTAGTC CCAGCTACTT GGGATGCTGA GGCAGGAAGA | 1680 |
| ATTGCTTGAA CCTGGGAGGC AGAGTCTGCG GTGACCGAGA TCATGCCACT GCACTCCAGC | 1740 |
| CTGGGTGACA GAGCCAGACT CCGTCTCTAC TAAAAAAAAA AAAAAAAAAA AAA | |
| Seq ID NO: 3 Protein sequence: | |
| Protein Accession #: CAA58280.1 | |
| 1 11 21 31 41 51 | |
| \| \| \| \| \| \| | |
| MDRSNPVKPA LDYFSNRLVN YQISVKCSNQ FKLEVCLLNA ENKVVDNQAG TQGQLKVLGA | 60 |
| NLWWPYLMHE HPAYLYSWED GDCSHQSLGP LPACDLWDQL HLRSRQGGSV CGCDPCEQLL | 120 |
| LLVSQLRAPG VDSAAAGRPV | |
| Seq ID NO: 4 DNA sequence | |
| Nucleic Acid Accession #: BC002622.1 | |
| 1 11 21 31 41 51 | |
| \| \| \| \| \| \| | |
| GGCACGAGGC TCCGCCCGCG GCCGGGATGC ACTAGGCAAA GCCAGCTGGG CTCCTGAGTC | 60 |
| CGGTGGGTAC TTGGAGAACT TACTACGTCT AGCTGGAGGA TTGTAAATGC ACCAATCAGC | 120 |
| ATGCTGTGTC TAGCTCAAGA TTTTCTCCAT CCCCTTATTT TGGGCCAGTG GCTGTCATTA | 180 |
| CATATGAGAA CTCAAGCTCC ATGAGGAGAT GTTTCATTGT CGAGAGCAGT CATGATGGCC | 240 |
| TGCACTCCAC ACAATGCAAC AGAGTGAAAG AGCAGGTTCT GCTTCTTTGG TGTAGTCCTG | 300 |
| AAGCTTCCTA AGAAACTTCA CATCAGGTGA TGGATAGGAG CAACCCTGTA AAACCAGCCT | 360 |
| TAGACTATTT TTCAAACAGG CTGGTGAATT ACCAGATCTC CGTCAAGTGC AGTAACCAGT | 420 |
| TCAAGTTGGA AGTGTGTCTT TTGAATGCAG AAAACAAAGT CGTGGACAAC CAGGCTGGGA | 480 |
| CCCAGGGCCA GCTGAAGGTG CTGGGTGCCA ACCTCTGGTG GCCGTACCTG ATGCACGAAC | 540 |
| ACCCCGCCTA CCTGTACTCG TGGGAGGATG GTGATTGCTC ACACCAAAGC CTTGGACCCC | 600 |
| TCCCAGCCTG TGACCTTTGT GACCAACTCC ACCTACGCAG CAGACAAGGG GGCTCTGTAT | 660 |
| GTGGATGTGA TCCGTGTGAA CAGCTACTAC TCTTGGTATC GCAACTACGG GCACCTGGAG | 720 |
| TTGATTCAGC TGCAGCTGGC CGCCCAGTTT GAGAATTGGT GTAAGACATC ACAATCCCAT | 780 |
| TATTCAGAGC GCGTATGGAG TGGAAACGCT TGTAGGGTTT CACCAGTCTT TCCCAGGGAA | 840 |
| CTCCGATGAA GTGTTCCAAC AAAATGAGCG AGTGAACCAA GAAGAGGATG ACATTAGATC | 900 |
| CAGGAGATAC AACAGAGGAG ATAATCTCCA GGATGCCTGT GAAGAAAGAT CCCTGGATCC | 960 |
| CAGGATGATT ATAGGACAAG TTGTTCATAA TCCAGCAGGC CAGAAGACTT CCAGGGAAAC | 1020 |
| TCATTCAAGG AGGTGAAAAT GATGGATGAC TCCTCCAAGA TGAAAATGGA CCAGCCGCAG | 1080 |
| TGGCTCACGC CTGTAATACC AGCACTTTGG GAGGCTGAGG CAGGCGGATC ACTTGAGGTC | 1140 |
| AGGAGTTTGA AACTAGCCTG GCCAACGTGG CAAAACTCCA TCTCTATTAA AAATACAAAA | 1200 |
| ATTAGCCAAG CATAGTGGTG CATGCCTGTA GTCCCAGCTA CTTGGGATGC TGAGGCAGGA | 1260 |
| AGAATTGCTT GAACCTGGGA GGCAGAGTCT ACAGTGAGCC GAGATCATGC CACTGCACTC | 1320 |
| CAGCCTGGGC AACACAGTGA GACTCCATCT CAAAAAAAAA AAAAAAAAAA AA | |
| Seq ID NO: 5 Protein sequence: | |
| Protein Accession #: AAH02622.1 | |
| 1 11 21 31 41 51 | |
| \| \| \| \| \| \| | |
| MDRSNPVKPA LDYFSNRLVN YQISVKCSNQ FKLEVCLLNA ENKVVDNQAG TQGQLKVLGA | 60 |
| NLWWPYLMHE HPAYLYSWED GDCSHQSLGP LPACDLCDQL HLRSRQGGSV CGCDPCEQLL | 120 |
| LLVSQLRAPG VDSAAAGRPV | |
| Seq ID NO: 6 DNA sequence | |
| Nucleic Acid Accession #: see Table 25 & 25A for complete list | |
| 1 11 21 31 41 51 | |
| \| \| \| \| \| \| | |
| ACCTGAGATC AGGAGTTCGA GATCAGCCTG ACCAATAGGG TGAAACCCCG TCTCTACTAA | 60 |
| AAATACAAAA AATTAGCTGG ACACGATGGT GGGTGCCTGT GGTCCCGGCT ACTCGGGAGG | 120 |
| CTGAGACAGG AGAATCAGTT GACCTGGGAG TTGGTGGTTG CAGTGAGCTG AGATCACACC | 180 |
| ATTGCATTCC AAGCCTGGGC AACAAGAGTG AAACTCCATC GCAAAAAAAA AAAAGAAGGG | 240 |
| GCATAATTTG TGGATGAGGA TTGGATATAA GGTAAAGGAT GGGACATTCT TGGACTTACA | 300 |
| GATGGTGTGA TTGCCTGGCT AGAAGAAGAA TTCCCGGTCA AAAAGAAACC ATCAGCTTTC | 360 |
| CAAGTGTGAA AGAGAGATAA ATCTGTGAAG ATTATAGGGA CTACAGGAAA CTTAATCTTT | 420 |
| TTCTTTGAAA AAGCAATTGT AGCAAAAAAA AAGAAAATTT CTTACTGTCA TCTAAAATTG | 480 |
| ACATGGACAT CTTAGTGGAC TAGAAGTTAA GGGCATAAAT TCTCCCAGTG ATTTTTAATT | 540 |
| TTAGCATTGT GATTAACACC TTCTAAAATT GCCAGAACTT AATAAATAAT TGCTTTTCAT | 600 |
| TATTAGTATG CCATCAAATT TAGTAGCTGT TTCAGGCTTT AATGTGTCAA GCCTAAAATC | 660 |
| CAGATTTTTG AGGATCTTCT CCCTCTTAAA AGAGTATTCA GTTAACTGCC GTAGAAATAC | 720 |
| ACATGTATAC AAGGGCACTG TATACATCAG TCTAAAAAAT AAAAATATGT ATACGTTCTG | 780 |
| GTGAGTCTAG CACAGCATTG CCCAATAGAA ATACCAATGG AGGTCACAAA TGTGGCCCAT | 840 |
| ATAGGTTAAT TGGTAAATTT TCTNATAGNC ACC | |
| Seq ID NO: 7 DNA sequence | |
| Nucleic Acid Accession #: AK000942 | |
| Coding sequence: 1204-1503 | |
| 1 11 21 31 41 51 | |
| \| \| \| \| \| \| | |
| GTAAAGGAAT GTCTTTTTAA TTCAGCTTTT CTTTTCTCCA TGCTAGTGTT ATCAGGTTTT | 60 |
| GGTATTTATT TACTTACAGC ATATGTTATG AAGCTGGTTT GAAAATTGGT TTTAGATATA | 120 |
| TCTGCAAGTT TACTACTTTG ACTGTAAAAA AAAAAAATGA AAAAGTAGTT GACATCTGTC | 180 |
| CTCAGAAGAA GTTTGCAGGT TGCATATTTG TGTGTAAATA CACAGGCTAA AAGGTAATTT | 240 |
| ATGTTCCTTG GGAATTGAAA TGGTCAGTGG CCCGTTACAG AAACTTATCA GTCATATATC | 300 |
| AGCACCAGTT CATTCTTTTG CACCTTAGGG ACCATCTGTC CCCTGAGGTG ACCTGAGAAA | 360 |
| CAACCAGTTG CCCACAGACT GTTATTTCTT CAAGTGAGCC AGGATTTGAT TTCACTGCCT | 420 |
| TATATTCTAT TTTTAGTGTA CAGTGCTTTG ATTTTTTGGA AAAACTAAAT TTTAAACATA | 480 |
| TTTGAAAAAT GTTATAAGAC TTGGACATTA AGTCTGTTGA TAGCCAAAGT CAGTTTACCA | 540 |
| AAGTAAAACA AATAAATTCT ATGCTTCTTC ATTGTCAAAG AGCAGTCTGC CATCATGTGG | 600 |
| ATATAAATGG ACTATGTAAA GTGACATGGT GCTTACTCTC TACCTAATAA TAGCCTCCCT | 660 |
| CCTGTTCCAA CAAGATAACC AACAGGTATA TTTAATTTAC CAGTTAATAT GTTTTGGATA | 720 |
| ATTGGCTGCC TTGAAATGCT ATATGTTTTA TAGTACATCA TAGCTTTAGT TTTCTTCATA | 780 |
| AGGAAATTAC AGTTACATCC TGGCTAACAT GGTGAAACTC CATCTCTACT AAAAATACAA | 840 |
| AAAATTAGCC GGGCGTGGTG GCGGGCACTT GTAGTCCCAG CTACTCGGGA GGCTGAGGCA | 900 |
| GGAGAATGGC GTGAACCCAG GAGGCGGAGG TTGCAGTGAG CCGAGATCGT GCCACTGTAC | 960 |
| TCTGGCCTGG GAGACAGAGC GAGACTCCAT CTCAAAAAAA AAAAAAAAAA AAAAAAAAGA | 1020 |
| GAGAGAGAGA CCTGGAGTAG AGATTCTGTC AAAGAACTTT TTCTTTCTTG AGAAGCATCT | 1080 |
| GAAATGGAAT CTGTTGTCTC TTCGAAATAT GTACTGCTGT AACAGTGAAA CAACCCTCAG | 1140 |
| AGTATGCCTT CGTGTGGGCT ACTCGTTGTG GTTTTGAACT TGGGGGAACT GTCTGTGTTT | 1200 |
| GGGTCAAGAA TATGCAACTG GCTGGGCACA TTGGCTCACG CCTGTAATCC CAGCAATTTG | 1260 |
| GGAGGCTGAG GCAGGCGGAT CACCTGAGGT CAGGGCTTCA AGACCAGACT GGCCAACATG | 1320 |
| GTGAAACCCC GTCTCTACTG AAAATACAAA AATTAGCTGG GCATGGTGGC AGGTGCCTGT | 1380 |
| AATCCCAGCT ACTCGGGAGG CTGACGTGAG AGAATCGCTT GAACCCGGGA GTTGGAGGTT | 1440 |
| GCAGTGAGCC GAGATTGCAC CATTGCACTC CAGCTTGGGC AACAAGAGTG AAACTCTTGT | 1500 |
| CTCAG | |
| Seq ID NO: 8 DNA sequence | |
| Nucleic Acid Accession #: see Table 25 & 25A for complete list | |
| 1 11 21 31 41 51 | |
| \| \| \| \| \| \| | |
| GACTAGGCTG GGCAACATAG TGAGACCTCA TCTCTAAAAT TAAAAAAATA AAAGCCACCA | 60 |
| GAAAAAAACC TAAAAACATG CCAAGTGACA TCAGTCTTTG ATGAAAATGG CAGCAGAAGA | 120 |
| GTGATGCCAT GGGTGGGGGT GGGAAATGCT ATTTCAGCAG AGAGGGAGCT GTCATGGAAG | 180 |
| ACACCATGTG GCTGGGCACG GTGGCTCACA CCTGTAATCC CAGCACTTTG GGAGATAGAG | 240 |
| GCAGGTGGAT CCCTTGAGCT TAGGAATTTG AGACTAGCCT GGGCAATAAG AGTGAAACTC | 300 |
| CATCTCAAAA AAAAAAAAAA AAAAAGGTGC ATGAAACATA TGAAGCAAAA AGTGAAAGTC | 360 |
| CCCATTCTTT TCCTTTTTCC AGAGGTGATT TTTGTGGCCA ATCTGGTTTC ATTCCCTCCC | 420 |
| AGACACTTTT CTAGGCATCT ATGCGCCTCT ATTCACATAT AAACAAAATA GGAGTTTTCC | 480 |
| TGTGCTTCCC TTAAATGGCA TATGTATCTT TCACTCTTTT TTTTCACCTA GTGGATCTTT | 540 |
| AATACCTTAA AAGCTCAACC TGGGCTTGGT GCGGTGGCTC ATACGTGTAA TCCCAGGCCT | 600 |
| TTGGGAGGCC AAGGTGGGAG GATCACTTGA GCTCAGGAGT TCCAGACCAT TCCAAAGCAA | 660 |
| AAACAAAAGG ATTTTGAGAT CAGTGTGGGC AACTTAGCAA AACACCATCT CTTAAAAAAA | 720 |
| AAAAAAAAAA | |
| Seq ID NO: 9 DNA sequence | |
| Nucleic Acid Accession #: BC010433.1 | |
| Coding sequence: 3-335 | |
| 1 11 21 31 41 51 | |
| \| \| \| \| \| \| | |
| GGTCGCCCTC CGTCGTGGTC TGGCGTGTAT TCCGAGCCTT GGTGTCTGGC GGTTTCCGAG | 60 |
| CGTTGGTGTC TGGCGGTTTC CGAGCGTTGG TGTCTGGCGG TTTCCGACCG TTGGTGTCTG | 120 |
| GCGGTTTCCG ACCGTTGGTG TCTGGCACGC GCCACCCTCT CTTGCTTTGG TTGCGCCATG | 180 |
| CCGATGTACC AGACAAGAAG ACAAGAAAAT GATTTGAGGA CAGCTTCAAT CGCGGTGTGA | 240 |
| AGAAGAAAGC AGCAAAACGA CCACTGAAAA CAACGCCGGT GGCAAAATAT CCAAAGAAAG | 300 |
| GGTCCCAAGC GGTACATCGT CATAGCCGGA AACAGTCAGA GCCACCAGCC AATGATCTTT | 360 |
| TCAATGCTGC GAAAGCTGCC AAAAGTGACA TGCAGCACCG AGAAGTCCGC GTGAAGTGCG | 420 |
| TGAAGGCTCT GAAAGGGCTG TACGGTAACC GGGACCTGAC CGCACGCCTG GAGCTCTTCA | 480 |
| CTGGCCGCTT CAAGGACTGG ATGGTTTCCA TGATCATGGA CAGAGAGTAC AGTGTGGCAG | 540 |
| TGGAGGCCGT CAGATTACTG ATACTTATCC TTAAGAACAT GGAAGGGGTG CTGATGGACG | 600 |
| TGGACTGTGA GAGCGTCTAC CCCATTGTGT AGGCCTCTAA TTGAGGCCTG GCCTCTGCTG | 660 |
| TGGGTGAATT TCTGTACTGG AAACTTTTCT ACCCTGAGTG CGAGATAAGA ACGATGGGTG | 720 |
| GAAGAGAGCA ACGCCAGAGC CCAGGTGCCC AGAGGACTTT CTTCCAGCTT CTGCTGTCCT | 780 |
| TCTTTGTGGA GAGCAAGCTC CACGACCACG CTGCTTACTT AGTAGACAAC CTGTGGGACT | 840 |
| GTGCAGGGAC TCAGCTGAAG GACTGGGAGG GTCTGACAAG CCTGCTGCTG GAGAAGGACC | 900 |
| AGAGCACGTG CCACATGGAG CCAGGGCCAG GGACCTTCCA CCTCCTAGGG TGAAACCAGG | 960 |
| AGAGATTGCT TGCTTCACTT GTACAAGGCA GGAACGGTGG CATGGGGTGG GGGAAACTTG | 1020 |
| GAGTTGGAAG GTGGCTAATC TTTGATTCTA TGTTTTTGAT CCTCCTGGCA CTCCAGACCT | 1080 |
| GGGTGATGTG CAGGAGAGCA CACTGATAGA AATCCTTGTG TCCAGTGCCC AGCAACTCCT | 1140 |
| GCCTCAGCCT CCCGAGCAGC TGGGACTACA GGCGCCCGCC ACCACGCCTG GCTAACTTTT | 1200 |
| TTGTGTTTTT AGTAGAGACG GGTTTTCACC GTGTTGGCCA GGATGGTCTT GATCTCTTGA | 1260 |
| CCTTGTGATC CACCTGCCTC ATCATCCCAA AGTGCTGGGA TTACAGGCGT GAGCCACTGC | 1320 |
| GCCCAGCATG TTAGACAATT TTTAATTCAT CCTCTCTGTG CTGTTGTTTT CTCAGCTGTG | 1380 |
| AAAGGAATAT TCTGGTGGGG ACAAGGTTAC AGAGTTGCTG AGAGGGTCTC ATGACATGAA | 1440 |
| GGTACTGGCC TTGGCACAGT GCCTGGGGGG GCGGGGACTC CGCACATGCC TGTGATGTCA | 1500 |
| CAGTTACTGT CAGTTCACAG CGAACCTTCC CTCCTTTTCC TGTTGACTTT CCCACACTCC | 1560 |
| TGTAACCCTC CCTCCCTCCC TTCTTCCTCT CTCTCTCTCT CACTCACGCA CACGCACACA | 1620 |
| CACACACACA CACACACACA CACACACTCC ATTCACTGTC TCCATGACTC TGGAGTAAAC | 1680 |
| TAACGTCTCG AGTFGCCATT GGAAGCCCCG TTGTCCTCAT TTAGACTTTC ATGGGTTATA | 1740 |
| GGCACTTTTG ACTTCCTGGG GTCCTTCTTC AGTTAAAAAA AAAAATTAGA AAATTAGGCC | 1800 |
| GGGCGTGGTG GCACATGCCT GTAATCCCAG CACCTTGGCC TCCCAAAGTG CTGGGATTAC | 1860 |
| AGGAGTGAGC CACCATGCCC AGCCTCCGTT GTCCTCATTT AGACTTTCAT GGGTTATAGG | 1920 |
| CACTTTTGAC TTCCTGGGGT CCTTCTTCAG TTAAAAAAAA AAAAAAAAAA | |
| Seq ID NO: 10 DNA sequence | |
| Nucleic Acid Accession #: see Table 25 & 25A for complete list | |
| 1 11 21 31 41 51 | |
| \| \| \| \| \| \| | |
| AGTGGNTCCC CCGGNCTGCA GGAATTCGGC ACGAGATCAT GATGGCTAAT ATTTCCTGAG | 60 |
| CACCTTTCAT TCAGGCATGA TGCCAGGTGC ACCAACTTAC TTAATCCTCA TAGCCACCAC | 120 |
| CTGAGCAAGC TCCTGTTTTA TAAATGGACC AGTTCTTGTT GCTGTTGTAC AAGTTATTTT | 180 |
| CTTTCTATAA CGTCCTCCTT GTCCTCCTTC CACATTCTTA AAGAAACTTT CCCTTCCTTT | 240 |
| AAAGTACTCA GGGAGCCCTG CATTGCTTCT TGAAGCCTTC TCCAGCTTCA TCATCTCACA | 300 |
| GTGGTCTCTC TTETCACTAA ATGTCCAATA TGCTGCACAT AAGTACCCCA AAGTTAGCAC | 360 |
| AGGAATTGTT CCATGGCTGT CATATATGTT AAAAATCATT AAAAGTTCAT TTTTTCTCTC | 420 |
| ATTATGGGAA GGATACATGC TCCTACTAGT AAATTTAGTA GGTAGAAAAA AATTATCACT | 480 |
| ATCTAGACTG CTTTCCATTT AGTCTTTATG CATAGCTTTC GTGTCTGCCT ATTTTTACCT | 540 |
| TGTGTTTGTA ACTTACTATT ATAAAATATG CGTCTCTATG TTCATTGTCA ACGATTATTT | 600 |
| ACAATAACAT GGAGTGGATT TACATGTATT CTCTATATTT GGATTAAAGG AGATAGAGTA | 660 |
| TGTGAAATTA AATGGGAGAA GTATCTGATA CATAACAGGC AATACAAATA TTATCACATA | 720 |
| GCGTCAATTT ATTTGTGAAT ATTGAAAGCT CCAAAAAAGA AAAAAAGTTT TTTTTTAATT | 780 |
| CCCGTAATTA CTTATTGCAG TATTGTGTTC ATACAAACTG CTCAGTCATT TTGGAGAAAT | 840 |
| AACAATTTTT TTCCTCATCA TGAAGTAAGG TATGCTCACT GCAAAAAAAA TCTAGAAAAT | 900 |
| AAAGAGGAAC ATGCTAAAGA AAAGAATACT CCCATATAAT CTCTGTCTTC ATAAATAATC | 960 |
| TTTTGTAACG CTTATACACT GCTGGTGGGA ATGTAAATTA GTTCAGCCAT TGTGAAAAGT | 1020 |
| AGCGTAGCAA TTCCTTGAAA AACTTAAAAT AGATTTACCG TTCAACCCAG CAATCCCATT | 1080 |
| ATTGGGCATA TACCCAGTGG AATGTAAATC ATCCTGCCAT AAAAACACAT GCACATGTAT | 1140 |
| GTTCATTGCA GCACTATTCA CAATAGCAAA GACATGGAAT CAACCTATAT GCCCATCAAT | 1200 |
| AGTAGACTGA ATAAAGAAAA TATGGTACAT ATTCACCACA GAATACTAAG CAGCCATAAA | 1260 |
| AAAAAA | |
| Seq ID NO: 11 DNA sequence | |
| Nucleic Acid Accession #: NM_000582.1 | |
| Coding sequence: 88-990 | |
| 1 11 21 31 41 51 | |
| \| \| \| \| \| \| | |
| GCAGAGCACA GCATCGTCGG GACCAGACTC GTCTCAGGCC AGTTGCAGCC TTCTCAGCCA | 60 |
| AACGCCGACC AAGGAAAACT CACTACCATG AGAATTGCAG TGATTTGCTT TTGCCTCCTA | 120 |
| GGCATCACCT GTGCCATACC AGTTAAACAG GCTGATTCTG GAAGTTCTGA GGAAAAGCAG | 180 |
| CTTTACAACA AATACCCAGA TGCTGTGGCC ACATGGCTAA ACCCTGACCC ATCTCAGAAG | 240 |
| CAGAATCTCC TAGCCCCACA GACCCTTCCA AGTAAGTCCA ACGAAAGCCA TGACCACATG | 300 |
| GATGATATGG ATGATGAAGA TGATGATGAC CATGTGGACA GCCAGGACTC CATTGACTCG | 360 |
| AACGACTCTG ATGATGTAGA TGACACTGAT GATTCTCACC AGTCTGATGA GTCTCACCAT | 420 |
| TCTGATGAAT CTGATGAACT GGTCACTGAT TTTCCCACGG ACCTGCCAGC AACCGAAGTT | 480 |
| TTCACTCCAG TTGTCCCCAC AGTAGACACA TATGATGGCC GAGGTGATAG TGTGGTTTAT | 540 |
| GGACTGAGGT CAAAATCTAA GAAGTTTCGC AGACCTGACA TCCAGTACCC TGATGCTACA | 600 |
| GACGAGGACA TCACCTCACA CATGGAAAGC GAGGAGTTGA ATGGTGCATA CAAGGCCATC | 660 |
| CCCGTTGCCC AGGACCTGAA CGCGCCTTCT GATTGGGACA GCCGTGGGAA GGACAGTTAT | 720 |
| GAAACGAGTC AGCTGGATGA CCAGAGTGCT GAAACCCACA GCCACAAGCA GTCCAGATTA | 780 |
| TATAAGCGGA AAGCCAATGA TGAGAGCAAT GAGCATTCCG ATGTGATTGA TAGTCAGGAA | 840 |
| CTTTCCAAAG TCAGCCGTGA ATTCCACAGC CATGAATTTC ACAGCCATGA AGATATGCTG | 900 |
| GTTGTAGACC CCAAAAGTAA GGAAGAAGAT AAACACCTGA AATTTCGTAT TTCTCATGAA | 960 |
| TTAGATAGTG CATCTTCTGA GGTCAATTAA AAGGAGAAAA AATACAATTT CTCACTTTGC | 1020 |
| ATTTAGTCAA AAGAAAAAAT GCTTTATAGC AAAATGAAAG AGAACATGAA ATGCTTCTTT | 1080 |
| CTCAGTTTAT TGGTTGAATG TGTATCTATT TGAGTCTGGA AATAACTAAT GTGTTTGATA | 1140 |
| ATTAGTTTAG TTTGTGGCTT CATGGAAACT CCCTGTAAAC TAAAAGCTTC AGGGTTATGT | 1200 |
| CTATGTTCAT TCTATAGAAG AAATGCAAAC TATCACTGTA TTTTAATATT TGTTATTCTC | 1260 |
| TCATGAATAG AAATTTATGT AGAAGCAAAC AAAATACTTT TACCCACTTA AAAAGAGAAT | 1320 |
| ATAACATTTT ATGTCACTAT AATCTTTTGT TTTTTAAGTT AGTGTATATT TTGTTGTGAT | 1380 |
| TATCTTTTTG TGGTGTGAAT AAATCTTTTA TCTTGAATGT AATAAGAATT TGGTGGTGTC | 1440 |
| AATTGCTTAT TTGTTTTCCC ACGGTTGTCC AGCAATTAAT AAAACATAAC CTTTTTTACT | 1500 |
| GCCTAAAAAA AAAAAAAAAA AAAA | |
| Seq ID NO: 12 Protein sequence: | |
| Protein Accession #: NP_000573.1 | |
| 1 11 21 31 41 51 | |
| \| \| \| \| \| \| | |
| MRIAVICFCL LGITCAIPVK QADSGSSEEK QLYNKYPDAV ATWLNPDPSQ KQNLLAPQTL | 60 |
| PSKSNESHDH MDDMDDEDDD DHVDSQDSID SNDSDDVDDT DDSHQSDESH HSDESDELVT | 120 |
| DFPTDLPATE VFTPVVPTVD TYDGRGDSVV YGLRSKSKKF RRPDIQYPDA TDEDITSHME | 180 |
| SEELNGAYKA IPVAQDLNAP SDWDSRGKDS YETSQLDDQS AETHSHKQSR LYKRKANDES | 240 |
| NEHSDVIDSQ ELSKVSREFH SHEFHSHEDM LVVDPKSKEE DKHLKFRISH ELDSASSEVN | |
| Seq ID NO: 13 DNA sequence | |
| Nucleic Acid Accession #: NM_001793 | |
| Coding sequence: 71-2560 | |
| 1 11 21 31 41 51 | |
| \| \| \| \| \| \| | |
| AAAGGGGCAA GAGCTGAGCG GAACACCGGC CCGCCGTCGC GGCAGCTGCT TCACCCCTCT | 60 |
| CTCTGCAGCC ATGGGGCTCC CTCGTGGACC TCTCGCGTCT CTCCTCCTTC TCCAGGTTTG | 120 |
| CTGGCTGCAG TGCGCGGCCT CCGAGCCGTG CCGGGCGGTC TTCAGGGAGG CTGAAGTGAC | 180 |
| CTTGGAGGCG GGAGGCGCGG AGCAGGAGCC CGGCCAGGCG CTGGGGAAAG TATTCATGGG | 240 |
| CTGCCCTGGG CAAGAGCCAG CTCTGTTTAG CACTGATAAT GATGACTTCA CTGTGCGGAA | 300 |
| TGGCGAGACA GTCCAGGAAA GAAGGTCACT GAAGGAAAGG AATCCATTGA AGATCTTCCC | 360 |
| ATCCAAACGT ATCTTACGAA GACACAAGAG AGATTGGGTG GTTGCTCCAA TATCTGTCCC | 420 |
| TGAAAATGGC AAGGGTCCCT TCCCCCAGAG ACTGAATCAG CTCAAGTCTA ATAAAGATAG | 480 |
| AGACACCAAG ATTTTCTACA GCATCACGGG GCCGGGGGCA GACAGCCCCC CTGAGGGTGT | 540 |
| CTTCGCTGTA GAGAAGGAGA CAGGCTGGTT GTTGTTGAAT AAGCCACTGG ACCGGGAGGA | 600 |
| GATTGCCAAG TATGAGCTCT TTGGCCACGC TGTGTCAGAG AATGGTGCCT CAGTGGAGGA | 660 |
| CCCCATGAAC ATCTCCATCA TCGTGACCGA CCAGAATGAC CACAAGCCCA AGTTTACCCA | 720 |
| GGACACCTTC CGAGGGAGTG TCTTAGAGGG AGTCCTACCA GGTACTTCTG TGATGCAGGT | 780 |
| GACAGCCACG GATGAGGATG ATGCCATCTA CACCTACAAT GGGGTGGTTG CTTACTCCAT | 840 |
| CCATAGCCAA GAACCAAAGG ACCCACACGA CCTCATGTTC ACCATTCACC GGAGCACAGG | 900 |
| CACCATCAGC GTCATCTCCA GTGGCCTGGA CCGGGAAAAA GTCCCTGAGT ACACACTGAC | 960 |
| CATCCAGGCC ACAGACATGG ATGGGGACGG CTCCACCACC ACGGCAGTGG CAGTAGTGGA | 1020 |
| GATCCTTGAT GCCAATGACA ATGCTCCCAT GTTTGACCCC CAGAAGTACG AGGCCCATGT | 1080 |
| GCCTGAGAAT GCAGTGGGCC ATGAGGTGCA GAGGCTGACG GTCACTGATC TGGACGCCCC | 1140 |
| CAACTCACCA GCGTGGCGTG CCACCTACCT TATCATGGGC GGTGACGACG GGGACCATTT | 1200 |
| TACCATCACC ACCCACCCTG AGAGCAACCA GGGCATCCTG ACAACCAGGA AGGGTTTGGA | 1260 |
| TTTTGAGGCC AAAAACCAGC ACACCCTGTA CGTTGAAGTG ACCAACGAGG CCCCTTTTGT | 1320 |
| GCTGAAGCTC CCAACCTCCA CAGCCACCAT AGTGGTCCAC GTGGAGGATG TGAATGAGGC | 1380 |
| ACCTGTGTTT GTCCCACCCT CCAAAGTCGT TGAGGTCCAG GAGGGCATCC CCACTGGGGA | 1440 |
| GCCTGTGTGT GTCTACACTG CAGAAGACCC TGACAAGGAG AATCAAAAGA TCAGCTACCG | 1500 |
| CATCCTGAGA GACCCAGCAG GGTGGCTAGC CATGGACCCA GACAGTGGGC AGGTCACAGC | 1560 |
| TGTGGGCACC CTCGACCGTG AGGATGAGCA GTTTGTGAGG AACAACATCT ATGAAGTCAT | 1620 |
| GGTCTTGGCC ATGGACAATG GAAGCCCTCC CACCACTGGC ACGGGAACCC TTCTGCTAAC | 1680 |
| ACTGATTGAT GTCAATGACC ATGGCCCAGT CCCTGAGCCC CGTCAGATCA CCATCTGCAA | 1740 |
| CCAAAGCCCT GTGCGCCAGG TGCTGAACAT CACGGACAAG GACCTGTCTC CCCACACCTC | 1800 |
| CCCTTTCCAG GCCCAGCTCA CAGATGACTC AGACATCTAC TGGACGGCAG AGGTCAACGA | 1860 |
| GGAAGGTGAC ACAGTGGTCT TGTCCCTGAA GAAGTTCCTG AAGCAGGATA CATATGACGT | 1920 |
| GCACCTTTCT CTGTCTGACC ATGGCAACAA AGAGCAGCTG ACGGTGATCA GGGCCACTGT | 1980 |
| GTGCGACTGC CATGGCCATG TCGAAACCTG CCCTGGACCC TGGAAGGGAG GTTTCATCCT | 2040 |
| CCCTGTGCTG GGGGCTGTCC TGGCTCTGCT GTTCCTCCTG CTGGTGCTGC TTTTGTTGGT | 2100 |
| GAGAAAGAAG CGGAAGATCA AGGAGCCCCT CCTACTCCCA GAAGATGACA CCCGTGACAA | 2160 |
| CGTCTTCTAC TATGGCGAAG AGGGGGGTGG CGAAGAGGAC CAGGACTATG ACATCACCCA | 2220 |
| GCTCCACCGA GGTCTGGAGG CCAGGCCGGA GGTGGTTCTC CGCAATGACG TGGCACCAAC | 2280 |
| CATCATCCCG ACACCCATGT ACCGTCCTCG GCCAGCCAAC CCAGATGAAA TCGGCAACTT | 2340 |
| TATAATTGAG AACCTGAAGG CGGCTAACAC AGACCCCACA GCCCCGCCCT ACGACACCCT | 2400 |
| CTTGGTGTTC GACTATGAGG GCAGCGGCTC CGACGCCGCG TCCCTGAGCT CCCTCACCTC | 2460 |
| CTCCGCCTCC GACCAAGACC AAGATTACGA TTATCTGAAC GAGTGGGGCA GCCGCTTCAA | 2520 |
| GAAGCTGGCA GACATGTACG GTGGCGGGGA GGACGACTAG GCGGCCTGCC TGCAGGGCTG | 2580 |
| GGGACCAAAC GTCAGGCCAC AGAGCATCTC CAAGGGGTCT CAGTTCCCCC TTCAGCTGAG | 2640 |
| GACTTCGGAG CTTGTCAGGA AGTGGCCGTA GCAACTTGGC GGAGACAGGC TATGAGTCTG | 2700 |
| ACGTTAGAGT GGTTGCTTCC TTAGCCTTTC AGGATGGAGG AATGTGGGCA GTTTGACTTC | 2760 |
| AGCACTGAAA ACCTCTCCAC CTGGGCCAGG GTTGCCTCAG AGGCCAAGTT TCCAGAAGCC | 2820 |
| TCTTACCTGC CGTAAAATGC TCAACCCTGT GTCCTGGGCC TGGGCCTGCT GTGACTGACC | 2880 |
| TACAGTGGAC TTTCTCTCTG GAATGGAACC TTCTTAGGCC TCCTGGTGCA ACTTAATTTT | 2940 |
| TTTTTTTAAT GCTATCTTCA AAACGTTAGA GAAAGTTCTT CAAAAGTGCA GCCCAGAGCT | 3000 |
| GCTGGGCCCA CTGGCCGTCC TGCATTTCTG GTTTCCAGAC CCCAATGCCT CCCATTCGGA | 3060 |
| TGGATCTCTG CGTTTTTATA CTGAGTGTGC CTAGGTTGCC CCTTATTTTT TATTTTCCCT | 3120 |
| GTTGCGTTGC TATAGATGAA GGGTGAGGAC AATCGTGTAT ATGTACTAGA ACTTTTTTAT | 3180 |
| TAAAGAAACT TTTCCCAGAA AAAAA | |
| Seq ID NO: 14 Protein sequence: | |
| Protein Accession #: NP_001784.2 | |
| 1 11 21 31 41 51 | |
| \| \| \| \| \| \| | |
| MGLPRGPLAS LLLLQVCWLQ CAASEPCRAV FREAEVTLEA GGAEQEPGQA LGKVFMGCPG | 60 |
| QEPALFSTDN DDFTVRNGET VQERRSLKER NPLKIFPSKR ILRRHKRDWV VAPISVPENG | 120 |
| KGPFPQRLNQ LKSNKDRDTK IFYSITGPGA DSPPEGVFAV EKETGWLLLN KPLDREEIAK | 180 |
| YELFGHAVSE NGASVEDPMN ISIIVTDQND HKPKFTQDTF RGSVLEGVLP GTSVMQVTAT | 240 |
| DEDDAIYTYN GVVAYSIHSQ EPKDPHDLMF TIHRSTGTIS VISSGLDREK VPEYTLTIQA | 300 |
| TDMDGDGSTT TAVAVVEILD ANDNAPMFDP QKYEAHVPEN AVGHEVQRLT VTDLDAPNSP | 360 |
| AWRATYLIMG GDDGDHFTIT THPESNQGIL TTRKGLDFEA KNQHTLYVEV TNEAPFVLKL | 420 |
| PTSTATIVVH VEDVNEAPVF VPPSKVVEVQ EGIPTGEPVC VYTAEDPDKE NQKISYRILR | 480 |
| DPAGWLAMDP DSGQVTAVGT LDREDEQFVR NNIYEVMVLA MDNGSPPTTG TGTLLLTLID | 540 |
| VNDHGPVPEP RQITICNQSP VRQVLNITDK DLSPHTSPFQ AQLTDDSDIY WTAEVNEEGD | 600 |
| TVVLSLKKFL KQDTYDVHLS LSDHGNKEQL TVIRATVCDC HGHVETCPGP WKGGFILPVL | 660 |
| GAVLALLFLL LVLLLLVRKK RKIKEPLLLP EDDTRDNVFY YGEEGGGEED QDYDITQLHR | 720 |
| GLEARPEVYL RNDVAPTIIP TPMYRPRPAN PDEIGNFIIE NLKAANTDPT APPYDTLLVF | 780 |
| DYEGSGSDAA SLSSLTSSAS DQDQDYDYLN EWGSRFKKLA DMYGGGEDD | |
| Seq ID NO: 15 DNA sequence | |
| Nucleic Acid Accession #: XM_051860.2 | |
| Coding sequence: 261-4346 | |
| 1 11 21 31 41 51 | |
| \| \| \| \| \| \| | |
| GAGCTAGCGC TCAAGCAGAG CCCAGCGCGG TGCTATCGGA CAGAGCCTGG CGAGCGCAAG | 60 |
| CGGCGCGGGG AGCCAGCGGG GCTGAGCGCG GCCAGGGTCT GAACCCAGAT TTCCCAGACT | 120 |
| AGCTACCACT CCGCTTGCCC ACGCCCCGGG AGCTCGCGGC GCCTGGCGGT CAGCGACCAG | 180 |
| ACGTCCGGGG CCGCTGCGCT CCTGGCCCGC GAGGCGTGAC ACTGTCTCGG CTACAGACCC | 240 |
| AGAGGGAGCA CACTGCCAGG ATGGGAGCTG CTGGGAGGCA GGACTTCCTC TTCAAGGCCA | 300 |
| TGCTGACCAT CAGCTGGCTC ACTCTGACCT GCTTCCCTGG GGCCACATCC ACAGTGGCTG | 360 |
| CTGGGTGCCC TGACCAGAGC CCTGAGTTGC AACCCTGGAA CCCTGGCCAT GACCAAGACC | 420 |
| ACCATGTGCA TATCGGCCAG GGCAAGACAC TGCTGCTCAC CTCTTCTGCC ACGGTCTATT | 480 |
| CCATCCACAT CTCAGAGGGA GGCAAGCTGG TCATTAAAGA CCACGACGAG CCGATTGTTT | 540 |
| TGCGAACCCG GCACATCCTG ATTGACAACG GAGGAGAGCT GCATGCTGGG AGTGCCCTCT | 600 |
| GCCCTTTCCA GGGCAATTTC ACCATCATTT TGTATGGAAG GGCTGATGAA GGTATTCAGC | 660 |
| CGGATCCTTA CTATGGTCTG AAGTACATTG GGGTTGGTAA AGGAGGCGCT CTTGAGTTGC | 720 |
| ATGGACAGAA AAAGCTCTCC TGGACATTTC TGAACAAGAC CCTTCACCCA GGTGGCATGG | 780 |
| CAGAAGGAGG CTATTTTTTT GAAAGGAGCT GGGGCCACCG TGGAGTTATT GTTCATGTCA | 840 |
| TCGACCCCAA ATCAGGCACA GTCATCCATT CTGACCGGTT TGACACCTAT AGATCCAAGA | 900 |
| AAGAGAGTGA ACGTCTGGTC CAGTATTTGA ACGCGGTGCC CGATGGCAGG ATCCTTTCTG | 960 |
| TTGCAGTGAA TGATGAAGGT TCTCGAAATC TGGATGACAT GGCCAGGAAG GCGATGACCA | 1020 |
| AATTGGGAAG CAAACACTTC CTGCACCTTG GATTTAGACA CCCTTGGAGT TTTCTAACTG | 1080 |
| TGAAAGGAAA TCCATCATCT TCAGTGGAAG ACCATATTGA ATATCATGGA CATCGAGGCT | 1140 |
| CTGCTGCTGC CCGGGTATTC AAATTGTTCC AGACAGAGCA TGGCGAATAT TTCAATGTTT | 1200 |
| CTTTGTCCAG TGAGTGGGTT CAAGACGTGG AGTGGACGGA GTGGTTCGAT CATGATAAAG | 1260 |
| TATCTCAGAC TAAAGGTGGG GAGAAAATTT CAGACCTCTG GAAAGCTCAC CCAGGAAAAA | 1320 |
| TATGCAATCG TCCCATTGAT ATACAGGCCA CTACAATGGA TGGAGTTAAC CTCAGCACCG | 1380 |
| AGGTTGTCTA CAAAAAAGGC CAGGATTATA GGTTTGCTTG CTACGACCGG GGCAGAGCCT | 1440 |
| GCCGGAGCTA CCGTGTACGG TTCCTCTGTG GGAAGCCTGT GAGGCCCAAA CTCACAGTCA | 1500 |
| CCATTGACAC CAATGTGAAC AGCACCATTC TGAACTTGGA GGATAATGTA CAGTCATGGA | 1560 |
| AACCTGGAGA TACCCTGGTC ATTGCCAGTA CTGATTACTC CATGTACCAG GCAGAAGAGT | 1620 |
| TCCAGGTGCT TCCCTGCAGA TCCTGCGCCC CCAACCAGGT CAAAGTGGCA GGGAAACCAA | 1680 |
| TGTACCTGCA CATCGGGGAG GAGATAGACG GCGTGGACAT GCGGGCGGAG GTTGGGCTTC | 1740 |
| TGAGCCGGAA CATCATAGTG ATGGGGGAGA TGGAGGACAA ATGCTACCCC TACAGAAACC | 1800 |
| ACATCTGCAA TTTCTTTGAC TTCGATACCT TTGGGGGCCA CATCAAGTTT GCTCTGGGAT | 1860 |
| TTAAGGCAGC ACACTTGGAG GGCACGGAGC TGAAGCATAT GGGACAGCAG CTGGTGGGTC | 1920 |
| AGTACCCGAT TCACTTCCAC CTGGCCGGTG ATGTAGACGA AAGGGGAGGT TATGACCCAC | 1980 |
| CCACATACAT CAGGGACCTC TCCATCCATC ATACATTCTC TCGCTGCGTC ACAGTCCATG | 2040 |
| GCTCCAATGG CTTGTTGATC AAGGACGTTG TGGGCTATAA CTCTTTGGGC CACTGCTTCT | 2100 |
| TCACGGAAGA TGGGCCGGAG GAACGCAACA CTTTTGACCA CTGTCTTGGC CTCCTTGTCA | 2160 |
| AGTCTGGAAC CCTCCTCCCC TCGGACCGTG ACAGCAAGAT GTGCAAGATG ATCACAGAGG | 2220 |
| ACTCCTACCC GGGGTACATC CCCAAGCCCA GGCAAGACTG CAATGCTGTG TCCACCTTCT | 2280 |
| GGATGGCCAA TCCCAACAAC AACCTCATCA ACTGTGCCGC TGCAGGATCT GAGGAAACTG | 2340 |
| GATTTTGGTT TATTTTTCAC CACGTACCAA CGGGCCCCTC CGTGGGAATG TACTCCCCAG | 2400 |
| GTTATTCAGA GCACATTCCA CTGGGAAAAT TCTATAACAA CCGAGCACAT TCCAACTACC | 2460 |
| GGGCTGGCAT GATCATAGAC AACGGAGTCA AAACCACCGA GGCCTCTGCC AAGGACAAGC | 2520 |
| GGCCGTTCCT CTCAATCATC TCTGCCAGAT ACAGCCCTCA CCAGGACGCC GACCCGCTGA | 2580 |
| AGCCCCGGGA GCCGGCCATC ATCAGACACT TCATTGCCTA CAAGAACCAG GACCACGGGG | 2640 |
| CCTGGCTGCG CGGCGGGGAT GTGTGGCTGG ACAGCTGCCG GTTTGCTGAC AATGGCATTG | 2700 |
| GCCTGACCCT GGCCAGTGGT GGAACCTTCC CGTATGACGA CGGCTCCAAG CAAGAGATAA | 2760 |
| AGAACAGCTT GTTTGTTGGC GAGAGTGGCA ACGTGGGGAC GGAAATGATG GACAATAGGA | 2820 |
| TCTGGGGCCC TGGCGGCTTG GACCATAGCG GAAGGACCCT CCCTATAGGC CAGAATTTTC | 2880 |
| CAATTAGAGG AATTCAGTTA TATGATGGCC CCATCAACAT CCAAAACTGC ACTTTCCGAA | 2940 |
| AGTTTGTGGC CCTGGAGGGC CGGCACACCA GCGCCCTGGC CTTCCGCCTG AATAATGCCT | 3000 |
| GGCAGAGCTG CCCCCATAAC AACGTGACCG GCATTGCCTT TGAGGACGTT CCGATTACTT | 3060 |
| CCAGAGTGTT CTTCGGAGAG CCTGGGCCCT GGTTCAACCA GCTGGACATG GATGGGGATA | 3120 |
| AGACATCTGT GYTCCATGAC GTCGACGGCT CCGTGTCCGA GTACCCTGGC TCCTACCTCA | 3180 |
| CGAAGAATGA CAACTGGCTG GTCCGGCACC CAGACTGCAT CAATGTTCCC GACTGGAGAG | 3240 |
| GGGCCATTTG CAGTGGGTGC TATGCACAGA TGTACATTCA AGCCTACAAG ACCAGTAACC | 3300 |
| TGCGAATGAA GATCATCAAG AATGACTTCC CCAGCCACCC TCTTTACCTG GAGGGGGCGC | 3360 |
| TCACCAGGAG CACCCATTAC CAGCAATACC AACCGGTTGT CACCCTGCAG AAGGGCTACA | 3420 |
| CCATCCACTG GGACCAGACG GCCCCCGCCG AACTCGCCAT CTGGCTCATC AACTTCAACA | 3480 |
| AGGGCGACTG GATCCGAGTG GGGCTCTGCT ACCCGCGAGG CACCACATTC TCCATCCTCT | 3540 |
| CGGATGTTCA CAATCGCCTG CTGAAGCAAA CGTCCAAGAC GGGCGTCTTC GTGAGGACCT | 3600 |
| TGCAGATGGA CAAAGTGGAG CAGAGCTACC CTGGCAGGAG CCACTACTAC TGGGACGAGG | 3660 |
| ACTCAGGGCT GTTGTTCCTG AAGCTGAAAG CTCAGAACGA GAGAGAGAAG TTTGCTTTCT | 3720 |
| GCTCCATGAA AGGCTGTGAG AGGATAAAGA TTAAAGCTCT GATTCCAAAG AACGCAGGCG | 3780 |
| TCAGTGACTG CACAGCCACA GCTTACCCCA AGTTCACCGA GAGGGCTGTC GTAGACGTGC | 3840 |
| CGATGCCCAA GAAGCTCTTT GGTTCTCAGC TGAAAACAAA GGACCATTTC TTGGAGGTGA | 3900 |
| AGATGGAGAG TTCCAAGCAG CACTTCTTCC ACCTCTGGAA CGACTTCGCT TACATTGAAG | 3960 |
| TGGATGGGAA GAAGTACCCC AGTTCGGAGG ATGGCATCCA GGTGGTGGTG ATTGACGGGA | 4020 |
| ACCAAGGGCG CGTGGTGAGC CACACGAGCT TCAGGAACTC CATTCTGCAA GGCATACCAT | 4080 |
| GGCAGCTTTT CAACTATGTG GCGACCATCC CTGACAATTC CATAGTGCTT ATGGCATCAA | 4140 |
| AGGGAAGATA CGTCTCCAGA GGCCCATGGA CCAGAGTGCT GGAAAAGCTT GGGGCAGACA | 4200 |
| GGGGTCTCAA GTTGAAAGAG CAAATGGCAT TCGTTGGCTT CAAAGGCAGC TTCCGGCCCA | 4260 |
| TCTGGGTGAC ACTGGACACT GAGGATCACA AAGCCAAAAT CTTCCAAGTT GTGCCCATCC | 4320 |
| CTGTGGTGAA GAAGAAGAAG TTGTGAGGAC AGCTGCCGCC CGGTGCCACC TCGTGGTAGA | 4380 |
| CTATGACGGT GACTCTTGGC AGCAGACCAG TGGGGGATGG CTGGGTCCCC CAGCCCCTGC | 4440 |
| CAGCAGCTGC CTGGGAAGGC CGTGTTTCAG CCCTGATGGG CCAAGGGAAG GCTATCAGAG | 4500 |
| ACCCTGGTGC TGCCACCTGC CCCTACTCAA GTGTCTACCT GGAGCCCCTG GGGCGGTGCT | 4560 |
| GGCCAATGCT GGAAACATTC ACTTTCCTGC AGCCTCTTGG GTGCTTCTCT CCTATCTGTG | 4620 |
| CCTCTTCAGT GGGGGTTTGG GGACCATATC AGGAGACCTG GGTTGTGCTG ACAGCAAAGA | 4680 |
| TCCACTTTGG CAGGAGCCCT GACCCAGCTA GGAGGTAGTC TGGAGGGCTG GTCATTCACA | 4740 |
| GATCCCCATG GTCTTCAGCA GACAAGTGAG GGTGGTAAAT GTAGGAGAAA GAGCCTTGGC | 4800 |
| CTTAAGGAAA TCTTTACTCC TGTAAGCAAG AGCCAACCTC ACAGGATTAG GAGCTGGGGT | 4860 |
| AGAACTGGCT ATCCTTGGGG AAGAGGCAAG CCCTGCCTCT GGCCGTGTCC ACCTTTCAGG | 4920 |
| AGACTTTGAG TGGCAGGTTT GGACTTGGAC TAGATGACTC TCAAAGGCCC TTTTAGTTCT | 4980 |
| GAGATTCCAG AAATCTGCTG CATTTCACAT GGTACCTGGA ACCCAACAGT TCATGGATAT | 5040 |
| CCACTGATAT CCATGATGCT GGGTGCCCCA GCGCACACGG GATGGAGAGG TGAGAACTAA | 5100 |
| TGCCTAGCTT GAGGGGTCTG CAGTCCAGTA GGGCAGGCAG TCAGGTCCAT GTGCACTGCA | 5160 |
| ATGCCAGGTG GAGAAATCAC AGAGAGGTAA AATGGAGGCC AGTGCCATTT CAGAGGGGAG | 5220 |
| GCTCAGGAAG GCTTCTTGCT TACAGGAATG AAGGCTGGGG GCATTTTGCT GGGGGGAGAT | 5280 |
| GAGGCAGCCT CTGGAATGGC TCAGGGATTC AGCCCTCCCT GCCGCTGCCT GCTGAAGCTG | 5340 |
| GTGACTACGG GGTCGCCCTT TGCTCACGTC TCTCTGGCCC ACTCATGATG GAGAAGTGTG | 5400 |
| GTCAGAGGGG AGCAATGGGC TTTGCTGCTT ATGAGCACAG AGGAATTCAG TCCCCAGGCA | 5460 |
| GCCCTGCCTC TGACTCCAAG AGGGTGAAGT CCACAGAAGT GAGCTCCTGC CTTAGGGCCT | 5520 |
| CATTTGCTCT TCATCCAGGG AACTGAGCAC AGGGGGCCTC CAGGAGACCC TAGATGTGCT | 5580 |
| CGTACTCCCT CGGCCTGGGA TTTCAGAGCT GGAAATATAG AAAATATCTA GCCCAAAGCC | 5640 |
| TTCATTTTAA CAGATGGGGA AAGTGAGCCC CCAAGATGGG AAAGAACCAC ACAGCTAAGG | 5700 |
| GAGGGCCTGG GGAGCCCCAC CCTAGCCCTT GCTGCCACAC CACATTGCCT CAACAACCGG | 5760 |
| CCCCAGAGTG CCCAGGCACT CCTGAGGTAG CTTCTGGAAA TGGGGACAAG TCCCCTCGAA | 5820 |
| GGAAAGGAAA TGACTAGAGT AGAATGACAG CTAGCAGATC TCTTCCCTCC TGCTCCCAGC | 5880 |
| GCACACAAAC CCGCCCTCCC CTTGGTGTTG GCGGTCCCTG TGGCCTTCAC TTTGTTCACT | 5940 |
| ACCTGTCAGC CCAGCCTGGG TGCACAGTAG CTGCAACTCC CCATTGGTGC TACCTGGCTC | 6000 |
| TCCTGTCTCT GCAGCTCTAC AGGTGAGGCC CAGCAGAGGG AGTAGGGCTC GCCATGTTTC | 6060 |
| TGGTGAGCCA ATTTGGCTGA TCTTGGGTGT CTGAACAGCT ATTGGGTCCA CCCCAGTCCC | 6120 |
| TTTCAGCTGC TGCTTAATGC CCTGCTCTCT CCCTGGCCCA CCTTATAGAG AGCCCAAAGA | 6180 |
| GCTCCTGTAA GAGGGAGAAC TCTATCTGTG GTTTATAATC TTGCACGAGG CACCAGAGTC | 6240 |
| TCCCTGGGTC TTGTGATGAA CTACATTTAT CCCCTTTCCT GCCCCAACCA CAAACTCTTT | 6300 |
| CCTTCAAAGA GGGCCTGCCT GGCTCCCTCC ACCCAACTGC ACCCATGAGA CTCGGTCCAA | 6360 |
| GAGTCCATTC CCCAGGTGGG AGCCAACTGT CAGGGAGGTC TTTCCCACCA AACATCTTTC | 6420 |
| AGCTGCTGGG AGGTGACCAT AGGGCTCTGC TTTTAAAGAT ATGGCTGCTT CAAAGGCCAG | 6480 |
| AGTCACAGGA AGGACTTCTT CCAGGGAGAT TAGTGGTGAT GGAGAGGAGA GTTAAAATGA | 6540 |
| CCTCATGTCC TTCTTGTCCA CGGTTTTGTT GAGTTTTCAC TCTTCTAATG CAAGGGTCTC | 6600 |
| ACACTGTGAA CCACTTAGGA TGTGATCACT TTCAGGTGGC CAGGAATGTT GAATGTCTTT | 6660 |
| GGCTCAGTTC ATTTAAAAAA GATATCTATT TGAAAGTTCT CAGAGTTGTA CATATGTTTC | 6720 |
| ACAGTACAGG ATCTGTACAT AAAAGTTTCT TTCCTAAACC ATTCACCAAG AGCCAATATC | 6780 |
| TAGGCATTTT CTTGGTAGCA CAAATTTTCT TATTGCTTAG AAAATTGTCC TCCTTGTTAT | 6840 |
| TTCTGTTTGT AAGACTTAAG TGAGTTAGGT CTTTAAGGAA AGCAACGCTC CTCTGAAATG | 6900 |
| CTTGTCTTTT TTCTGTTGCC GAAATAGCTG GTCCTTTTTC GGGAGTTAGA TGTATAGAGT | 6960 |
| GTTTGTATGT AAACATTTCT TGTAGGCATC ACCATGAACA AAGATATATT TTCTATTTAT | 7020 |
| TTATTATATG TGCACTTCAA GAAGTCACTG TCAGAGAAAT AAAGAATTGT CTTAAATGTC | |
| Seq ID NO: 16 Protein sequence: | |
| Protein Accession #: XP_051860.2 | |
| 1 11 21 31 41 51 | |
| \| \| \| \| \| \| | |
| MGAAGRQDFL FKAMLTISWL TLTCFPGATS TVAAGCPDQS PELQPWNPGH DQDHHVHIGQ | 60 |
| GKTLLLTSSA TVYSIHISEG GKLVIKDHDE PIVLRTRHIL IDNGGELHAG SALCPFQGNF | 120 |
| TIILYGRADE GIQPDPYYGL KYIGVGKGGA LELHGQKKLS WTFLNKTLHP GGMAEGGYFF | 180 |
| ERSWGHRGVI VHVIDPKSGT VIHSDRFDTY RSKKESERLV QYLNAVPDGR ILSVAVNDEG | 240 |
| SRNLDDMARK AMTKLGSKHF LHLGFRHPWS FLTVKGNPSS SVEDHIEYHG HRGSAAARVF | 300 |
| KLFQTEHGEY FNVSLSSEWV QDVEWTEWFD HDKVSQTKGG EKISDLWKAH PGKICNRPID | 360 |
| IQATTMDGVN LSTEVVYKKG QDYRFACYDR GRACRSYRVR FLCGKPVRPK LTVTIDTNVN | 420 |
| STILNLEDNV QSWKPGDTLV IASTDYSMYQ AEEFQVLPCR SCAPNQVKVA GKPMYLHIGE | 480 |
| EIDGVDMRAE VGLLSRNIIV MGEMEDKCYP YRNHICNFFD FDTFGGHIKF ALGFKAAHLE | 540 |
| GTELKHMGQQ LVGQYPIHFH LAGDVDERGG YDPPTYIRDL SIHHTFSRCV TVHGSNGLLI | 600 |
| KDVVGYNSLG HCFFTEDGPE ERNTFDHCLG LLVKSGTLLP SDRDSKMCKM ITEDSYPGYI | 660 |
| PKPRQDCNAV STFWMANPNN NLINCAAAGS EETGFWFIFH HVPTGPSVGM YSPGYSEHIP | 720 |
| LGKFYNNRAH SNYRAGMIID NGVKTTEASA KDKRPFLSII SARYSPHQDA DPLKPREPAI | 780 |
| IRHFIAYKNQ DHGAWLRGGD VWLDSCRFAD NGIGLTLASG GTFPYDDGSK QEIKNSLFVG | 840 |
| ESGNVGTEMM DNRIWGPGGL DHSGRTLPIG QNFPIRGIQL YDGPINIQNC TFRKFVALEG | 900 |
| RHTSALAFRL NNAWQSCPHN NVTGIAFEDV PITSRVFFGE PGPWFNQLDM DGDKTSVFHD | 960 |
| VDGSVSEYPG SYLTKNDNWL VRHPDCINVP DWRGAICSGC YAQMYIQAYK TSNLRMKIIK | 1020 |
| NDFPSHPLYL EGALTRSTHY QQYQPVVTLQ KGYTIHWDQT APAELAIWLI NFNKGDWIRV | 1080 |
| GLCYPRGTTF SILSDVHNRL LKQTSKTGVF VRTLQMDKVE QSYPGRSHYY WDEDSGLLFL | 1140 |
| KLKAQNEREK FAFCSMKGCE RIKIKALIPK NAGVSDCTAT AYPKFTERAV VDVPMPKKLF | 1200 |
| GSQLKTKDHF LEVKMESSKQ HFFHLWNDFA YIEVDGKKYP SSEDGIQVVV IDGNQGRVVS | 1260 |
| HTSFRNSILQ GIPWQLFNYV ATIPDNSIVL MASKGRYVSR GPWTRVLEKL GADRGLKLKE | 1320 |
| QMAFVGFKGS FRPIWVTLDT EDHKAKIFQV VPIPVVKKKK L | |
| Seq ID NO: 17 DNA sequence | |
| Nucleic Acid Accession #: NM_015515.1 | |
| Coding sequence: 61-1329 | |
| 1 11 21 31 41 51 | |
| \| \| \| \| \| \| | |
| AGTTCTGCGG TGCCAGGGAG TGGAGCAGAG CTCAGCCCCG TCCCAAACAC AGATGGGACC | 60 |
| ATGAACTCCG GACACAGCTT CAGCCAGACC CCCTCGGCCT CCTTCCATGG CGCCGGAGGT | 120 |
| GGCTGGGGCC GGCCCAGGAG CTTCCCCAGG GCTCCCACCG TCCATGGCGG TGCGGGGGGA | 180 |
| GCCCGCATCT CCCTGTCCTT CACCACGCGG AGCTGCCCAC CCCCTGGAGG GTCTTGGGGT | 240 |
| TCTGGAAGAA GCAGCCCCCT ACTAGGCGGA AATGGGAAGG CCACCATGCA GAATCTCAAC | 300 |
| GACCGCCTGG CCTCCTACCT GGAGAAGGTT CGCGCCCTGG AGGAGGCCAA CATGAAGCTG | 360 |
| GAAAGCCGCA TCCTGAAATG GCACCAGGAG AGAGATCCTG GCAGTAAGAA AGATTATTCC | 420 |
| CAGTATGAGG AAAACATCAC ACACCTGCAG GAGCAGATAG TGGATGGTAA GATGACCAAT | 480 |
| GCTCAGATTA TTCTTCTCAT TGACAATGCC AGGATGGCAG TGGATGACTT CAACCTCAAG | 540 |
| TATGAAAATG AACACTCCTT TAAGAAAGAC TTGGAAATTG AAGTCGAGGG CCTCCGAAGG | 600 |
| ACCTTAGACA ACCTGACCAT TGTCACAACA GACCTAGAAC AGGAGGTGGA AGGAATGAGG | 660 |
| AAAGAGCTCA TTCTCATGAA GGAGCACCAT GAGCAGGAAA TGGAGGAGCA TCATGTGCCA | 720 |
| AGTGACTTCA ATGTCAATGT GAAGGTGGAT ACAGGTCCCA GGGAAGATCT GATTAAGGTC | 780 |
| CTGGAGGATA TGAGACAAGA ATATGAGCTT ATAATAAAGA AGAAGCATCG AGACTTGGAC | 840 |
| ACTTGGTATA AAGAACAGTC TGCAGCCATG TCCCAGGAGG CAGCCAGTCC AGCCACTGTG | 900 |
| CAGAGCAGAC AAGGTGACAT CCACGAACTG AAGCGCACAT TCCAGGCCCT GGAGATTGAC | 960 |
| CTGCAGGCAC AGTACAGCAC GAAATCTGCT TTGGAAAACA TGTTATCCGA GACCCAGTCT | 1020 |
| CGGTACTCCT GCAAGCTCCA GGACATGCAA GAGATCATCT CCCACTATGA GGAGGAACTG | 1080 |
| ACGCAGCTAC GCCACGAACT GGAGCGGCAG AACAATGAAT ACCAAGTGCT GCTGGGCATC | 1140 |
| AAAACCCACC TGGAGAAGGA AATCACCACG TACCGACGGC TCCTGGAGGG AGAGAGTGAA | 1200 |
| GGGACACGGG AAGAATCAAA GTCGAGCATG AAAGTGTCTG CAACTCCAAA GATCAAGGCC | 1260 |
| ATAACCCAGG AGACCATCAA CGGAAGATTA GTTCTTTGTC AAGTGAATGA AATCCAAAAG | 1320 |
| CACGCATGAG ACCAATGAAA GTTTCCGCCT GTTGTAAAGT CTATTTTCCC CCAAGGAAAG | 1380 |
| TCCTTGCACA GACACCAGTG AGTGAGTTCT AAAAGATACC CTTGGAATTA TCAGACTCAG | 1440 |
| AAACTTTTAT TTTTTTTTTT CTGTAACAGT CTCACCAGAC TTCTCATAAT GCTCTTAATA | 1500 |
| TATTGCACTT TTCTAATCAA AGTGCGAGTT TATGAGGGTA AAGCTCTACT TTCCTACTGC | 1560 |
| AGCCTTCAGA TTCTCATCAT TTTGCATCTA TTTTGTAGCC AATAAAACTC CGCACTAGC | |
| Seq ID NO: 18 Protein sequence: | |
| Protein Accession #: NP_056330.1 | |
| 1 11 21 31 41 51 | |
| \| \| \| \| \| \| | |
| MNSGHSFSQT PSASFHGAGG GWGRPRSFPR APTVHGGAGG ARISLSFTTR SCPPPGGSWG | 60 |
| SGRSSPLLGG NGKATMQNLN DRLASYLEKV RALEEANMKL ESRILKWHQQ RDPGSKKDYS | 120 |
| QYEENITHLQ EQIVDGKMTN AQIILLIDNA RMAVDDFNLK YENEHSFKKD LEIEVEGLRR | 180 |
| TLDNLTIVTT DLEQEVEGMR KELILMKEHH EQEMEEHHVP SDFNVNVKVD TGPREDLIKV | 240 |
| LEDMRQEYEL IIKKKHRDLD TWYKEQSAAM SQEAASPATV QSRQGDIHEL KRTFQALEID | 300 |
| LQAQYSTKSA LENMLSETQS RYSCKLQDMQ EIISHYEEEL TQLRHELERQ NNEYQVLLGI | 360 |
| KTHLEKEITT YRRLLEGESE GTREESKSSM KVSATPKIKA ITQETINGRL VLCQVNEIQK | 420 |
| HA | |
| Seq ID NO: 19 DNA sequence | |
| Nucleic Acid Accession #: see Table 25 & 25A for complete list | |
| 1 11 21 31 41 51 | |
| \| \| \| \| \| \| | |
| TTTTTTTTTT TTAAAAAAAA GAGGCTTGGT AAGTTTTTGA TACTTAGTTG ACTTTTAGCA | 60 |
| TTATCCAGCA TTFGTATTAT GAACCAGTGA GTACTGTAAT TTTTCTTTCC CTTTCAGAAA | 120 |
| GACTCAAAGG GAACATATAA ATGTTTCCTA TTTTTNNNNN NNNNNNNNNN NNNNNNNNNN | 180 |
| NNNNACCCAT CGTGCGATGA TCNNNNNNNN NNNNNNNNNN NNNNNTTGGG ATCCAGTTTC | 240 |
| AAATAAGGTA TGGGAAAAAC AGATGTTTTC ATTATCGCCA CTTAATCCTT ACTTCCGATT | 300 |
| ATAATTATAC ATGTTTGGCT GTAATAACTA TACTAAAGCA TGCTTGTGAA AGTAGACTTC | 360 |
| TACAAGGACA GAAAACCCAC AACAACAAAG ATCGATCACG AAAGACAAGG CATATTCATT | 420 |
| CATTAATTTA CTTCTCTTAG ACCCGGGACA TGTGGGACAA ATACTTTTGT CCTCATGGAT | 480 |
| GGCTTGATAA TTTATTTATA TGTTCTAGAG TCTGAGGATT TTCTTTCAGT GGCAGACAAC | 540 |
| AAAGGATGTT ACAATTTACT TCAAAATAAT ACAATCATGG TTTAATTTAC AGTGTAAATC | 600 |
| CATAACTATT TTATAGAGAT GGATTATCAT ACATGGGATT ATAAAAATAA CTTACCCATA | 660 |
| TGCTTGCAAA ATAGACTTTT CCTATTGGGA GGAACATCTT TTAACCTAAA ACGGATTTAT | 720 |
| TTCAGATGAA TTAGACAGTA CATTTTTCAG GAGAACCAGC CTTACTGGAT GATCTTTTGT | 780 |
| CAGGTTTGGA GGCCTCTTCT TTGTCTTTGC AACCATAACC CCTTTTCAGC TGAAGACCAC | 840 |
| TGGCCTTCAA CCCAAGCCAG GAGTTTGGCT CAAATGA | |
| Seq ID NO: 20 DNA sequence | |
| Nucleic Acid Accession #: D3205 1.1 | |
| Coding sequence: 72-1373 | |
| 1 11 21 31 41 51 | |
| \| \| \| \| \| \| | |
| GAATTCGAAC CAGGTGGCCA CCCGGTGTCG GTTTCATTTT CCTTTGGAAT TTCTGCTTTA | 60 |
| CAGACAGAAC AATGGCAGCC CGAGTACTTA TAATTGGCAG TGGAGGAAGG GAACATACGC | 120 |
| TGGCCTGGAA ACTTGCACAG TCTCATCATG TCAAACAAGT GTTGGTTGCC CCAGGAAACG | 180 |
| CAGGCACTGC CTGCTCTGAA AAGATTTCAA ATACCGCCAT CTCAATCAGT GACCACACTG | 240 |
| CCCTTGCTCA ATTCTGCAAA GAGAAGAAAA TTGAATTTGT AGTTGTTGGA CCAGAAGCAC | 300 |
| CTCTGGCTGC TGGGATTGTT GGGAACCTGA GGTCTGCAGG AGTGCAATGC TTTGGCCCAA | 360 |
| CAGCAGAAGC GGCTCAGTTA GAGTCCAGCA AAAGGTTTGC CAAAGAGTTT ATGGACAGAC | 420 |
| ATGGAATCCC AACCGCACAA TGGAAGGCTT TCACCAAACC TGAAGAAGCC TGCAGCTTCA | 480 |
| TTTTGAGTGC AGACTTCCCT GCTTTGGTTG TGAAGGCCAG TGGTCTTGCA GCTGGAAAAG | 540 |
| GGGTGATTGT TGCAAAGAGC AAAGAAGAGG CCTGCAAAGC TGTACAAGAG ATCATGCAGG | 600 |
| AGAAAGCCTT TGGGGCAGCT GGAGAAACAA TTGTCATTGA AGAACTTCTT GACGGAGAAG | 660 |
| AGGTGTCGTG TCTGTGTTTC ACTGATGGCA AGACTGTGGC CCCCATGCCC CCAGCACAGG | 720 |
| ACCATAAGCG ATTACTGGAG GGAGATGGTG GCCCTAACAC AGGGGGAATG GGAGCCTATT | 780 |
| GTCCAGCCCC TCAGGTTTCT AATGATCTAT TACTAAAAAT TAAAGATACT GTTCTTCAGA | 840 |
| GGACAGTGGA TGGCATGCAG CAAGAGGGTA CTCCATATAC AGGTATTCTC TATGCTGGAA | 900 |
| TAATGCTGAC CAAGAATGGC CCAAAAGTTC TAGAGTTTAA TFGCCGTTTT GGTGATCCAG | 960 |
| AGTGCCAAGT AATCCTCCCA CTTCTTAAAA GTGATCTTTA TGAAGTGATT CAGTCCACCT | 1020 |
| TAGATGGACT GCTCTGCACA TCTCTGCCTG TTTGGCTAGA AAACCACACC GCCCTAACTG | 1080 |
| TTGTCATGGC AAGTAAAGGT TATCCTGGAG ACTACACCAA GGGTGTAGAG ATAACAGGGT | 1140 |
| TTCCTGAGGC TCAAGCTCTA GGACTGGAGG TGTCCCATGC AGGCACTGCC CTCAAAAATG | 1200 |
| GCAAAGTAGT AACTCATGGG GGTAGAGTTC TTGCAGTCAC AGCCATCCGG GAAAATCTCA | 1260 |
| TATCAGCCCT TGAGGAAGCC AAGAAAGGAC TAGCTGCTAT AAAGTTTGAG GGAGCAATTT | 1320 |
| ATAGGAAAGA CATCGGCTTT CGTGCCATAG CTTTCCTCCA GCAGCCCAGG TAAAACTCTA | 1380 |
| AGCAAGTTAG CTGTAGTGCC ATTTCAGAAA CTGGCCTAAA TGGCTATGTA GAACATTCCA | 1440 |
| TTAACCCTAT AAGTCATTCA GTATTCTTTT CTCTCTGTGG GAGTGATACA GTCTTGGTTT | 1500 |
| GTATTTTGTT TGAATCAAAA CTGGTTATAG CAATACTCAA ATGGAAAAAA CTTCATGATA | 1560 |
| GCGTAAGTTT GGAAAGTTTA GCAAAATCAC AGTGGTACTG ATTTTTATTT GTTTTCTATT | 1620 |
| TTTTTTATTT TATATTTTTA ATTTTTTTAA CAGGGTCTTC CTCTCTCGCC CAAGTTCTCA | 1680 |
| TGCCTCAGCC TCCCAAATAG CTGGGACTAC AGGCACAGGC CACCACACCT GGCTAATTTT | 1740 |
| TTTGTATTTT TTGTGGAGAT GGGGTTCACC ATGTTGCCAA GGCCAGTCTG AAAGCCTGGG | 1800 |
| CTCAAGTGAT CCTCCTGCTT TGGCCTCCCA AAATGCTGGG ACTATAGGCA TGAGGCGCTG | 1860 |
| CACTTGGCCT GATACTGATT TTTATTCCTT GCGTTATCAC ATAGTGTTGT ATTTGAAACA | 1920 |
| TAGTTCATGG TTTTATCAAA GAACTGAAGA TGAGAATACT GGTCATCTAA CTTTGTAATT | 1980 |
| TGATTTGATT ATACTGTAAA GTTTGACAGT CCCATTTTAA CCTGCGTTTG TATCTATTAC | 2040 |
| TAAAATGTAT TTTTTGACCT CTTACTGATT CATGGTTGGT ATGTACAAAC TGTTGACTTG | 2100 |
| TAAAATCAAT AAAGTCTTAG TTGG | |
| Seq ID NO: 21 Protein sequence: | |
| Protein Accession #: BA.A06809.1 | |
| 1 11 21 31 41 51 | |
| \| \| \| \| \| \| | |
| MAARVLIIGS GGREHTLAWK LAQSHHVKQV LVAPGNAGTA CSEKISNTAI SISDHTALAQ | 60 |
| FCKEKKIEFV VVGPEAPLAA GIVGNLRSAG VQCFGPTAEA AQLESSKRFA KEFMDRHGIP | 120 |
| TAQWKAFTKP EEACSFILSA DFPALVVKAS GLAAGKGVIV AKSKEEACKA VQEIMQEKAF | 180 |
| GAAGETIVIE ELLDGEEVSC LCFTDGKTVA PMPPAQDHKR LLEGDGGPNT GGMGAYCPAP | 240 |
| QVSNDLLLKI KDTVLQRTVD GMQQEGTPYT GILYAGIMLT KNGPKVLEFN CRFGDPECQV | 300 |
| ILPLLKSDLY EVIQSTLDGL LCTSLPVWLE NHTALTVVMA SKGYPGDYTK GVEITGFPEA | 360 |
| QALGLEVSHA GTALKNGKVV THGGRVLAVT AIRENLISAL EEAKKGLAAI KFEGAIYRKD | 420 |
| IGFRAIAFLQ QPR | |
| Seq ID NO: 22 DNA sequence | |
| Nucleic Acid Accession #: EOS cloned | |
| Coding sequence: 1-2424 | |
| 1 11 21 31 41 51 | |
| \| \| \| \| \| \| | |
| ATGCCCCCTT TCCTGTTGCT GGAGGCCGTC TGTGTTTTCC TGTTTTCCAG AGTGCCCCCA | 60 |
| TCTCTCCCTC TCCAGGAAGT CCATGTAAGC AAAGAAACCA TCGGGAAGAT TTCAGCTGCC | 120 |
| AGCAAAATGA TGTGGTGCTC GGCTGCAGTG GACATCATGT TTCTGTTAGA TGGGTCTAAC | 180 |
| AGCGTCGGGA AAGGGAGCTT TGAAAGGTCC AAGCACTTTG CCATCACAGT CTGTGACGGT | 240 |
| CTGGACATCA GCCCCGAGAG GGTCAGAGTG GGAGCATTCC AGTTCAGTTC CACTCCTCAT | 300 |
| CTGGAATTCC CCTTGGATTC ATTTTCAACC CAACAGGAAG TGAAGGCAAG AATCAAGAGG | 360 |
| ATGGTTTTCA AAGGAGGGCG CACGGAGACG GAACTTGCTC TGAAATACCT TCTGCACAGA | 420 |
| GGGTTGCCTG GAGGCAGAAA TGCTTCTGTG CCCCAGATCC TCATCATCGT CACTGATGGG | 480 |
| AAGTCCCAGG GGGATGTGGC ACTGCCATCC AAGCAGCTGA AGGAAAGGGG TGTCACTGTG | 540 |
| TTTGCTGTGG GGGTCAGGTT TCCCAGGTGG GAGGAGCTGC ATGCACTGGC CAGCGAGCCT | 600 |
| AGAGGGCAGC ACGTGCTGTT GGCTGAGCAG GTGGAGGATG CCACCAACGG CCTCTTCAGC | 660 |
| ACCCTCAGCA GCTCGGCCAT CTGCTCCAGC GCCACGCCAG ACTGCAGGGT CGAGGCTCAC | 720 |
| CCCTGTGAGC ACAGGACGCT GGAGATGGTC CGGGAGTTCG CTGGCAATGC CCCATGCTGG | 780 |
| AGAGGATCGC GGCGGACCCT TGCGGTGCTG GCTGCACACT GTCCCTTCTA CAGCTGGAAG | 840 |
| AGAGTGTTCC TAACCCACCC TGCCACCTGC TACAGGACCA CCTGCCCAGG CCCCTGTGAC | 900 |
| TCGCAGCCCT GCCAGAATGG AGGCACATGT GTTCCAGAAG GACTGGACGG CTACCAGTGC | 960 |
| CTCTGCCCGC TGGCCTTTGG AGGGGAGGCT AACTGTGCCC TGAAGCTGAG CCTGGAATGC | 1020 |
| AGGGTCGACC TCCTCTTCCT GCTGGACAGC TCTGCGGGCA CCACTCTGGA CGGCTTCCTG | 1080 |
| CGGGCCAAAG TCTTCGTGAA GCGGTTTGTG CGGGCCGTGC TGAGCGAGGA CTCTCGGGCC | 1140 |
| CGAGTGGGTG TGGCCACATA CAGCAGGGAG CTGCTGGTGG CGGTGCCTGT GGGGGAGTAC | 1200 |
| CAGGATGTGC CTGACCTGGT CTGGAGCCTC GATGGCATTC CCTTCCGTGG TGGCCCCACC | 1260 |
| CTGACGGGCA GTGCCTTGCG GCAGGCGGCA GAGCGTGGCT TCGGGAGCGC CACCAGGACA | 1320 |
| GGCCAGGACC GGCCACGTAG AGTGGTGGTT TTGCTCACTG AGTCACACTC CGAGGATGAG | 1380 |
| GTTGCGGGCC CAGCGCGTCA CGCAAGGGCG CGAGAGCTGC TCCTGCTGGG TGTAGGCAGT | 1440 |
| GAGGCCGTGC GGGCAGAGCT GGAGGAGATC ACAGGCAGCC CAAAGCATGT GATGGTCTAC | 1500 |
| TCGGATCCTC AGGATCTGTT CAACCAAATC CCTGAGCTGC AGGGGAAGCT GTGCAGCCGG | 1560 |
| CAGCGGCCAG GGTGCCGGAC ACAAGCCCTG GACCTCGTCT TCATGTTGGA CACCTCTGCC | 1620 |
| TCAGTAGGGC CCGAGAATTT TGCTCAGATG CAGAGCTTTG TGAGAAGCTG TGCCCTCCAG | 1680 |
| TTTGAGGTGA ACCCTGACGT GACACAGGTC GGCCTGGTGG TGTATGGCAG CCAGGTGCAG | 1740 |
| ACTGCCTTCG GGCTGGACAC CAAACCCACC CGGGCTGCGA TGCTGCGGGC CATTAGCCAG | 1800 |
| GCCCCCTACC TAGGTGGGGT GGGCTCAGCC GGCACCGCCC TGCTGCACAT CTATGACAAA | 1860 |
| GTGATGACCG TCCAGAGGGG TGCCCGGCCT GGTGTCCCCA AAGCTGTGGT GGTGCTCACA | 1920 |
| GGCGGGAGAG GCGCAGAGGA TGCAGCCGTT CCTGCCCAGA AGCTGAGGAA CAATGGCATC | 1980 |
| TCTGTCTTGG TCGTGGGCGT GGGGCCTGTC CTAAGTGAGG GTCTGCGGAG GCTTGCAGGT | 2040 |
| CCCCGGGATT CCCTGATCCA CGTGGCAGCT TACGCCGACC TGCGGTACCA CCAGGACGTG | 2100 |
| CTCATTGAGT GGCTGTGTGG AGAAGCCAAG CAGCCAGTCA ACCTCTGCAA ACCCAGCCCG | 2160 |
| TGCATGAATG AGGGCAGCTG CGTCCTGCAG AATGGGAGCT ACCGCTGCAA GTGTCGGGAT | 2220 |
| GGCTGGGAGG GCCCCCACTG CGAGAACCGT GAGTGGAGCT CTTGCTCTGT ATGTGTGAGC | 2280 |
| CAGGGATGGA TTCTTGAGAC GCCCCTGAGG CACATGGCTC CCGTGCAGGA GGGCAGCAGC | 2340 |
| CGTACCCCTC CCAGCAACTA CAGAGAAGGC CTGGGCACTG AAATGGTGCC TACCTTCTGG | 2400 |
| AATGTCTGTG CCCCAGGTCC TTAG | |
| Seq ID NO: 23 Protein sequence: | |
| Protein Accession #: EOS cloned | |
| 1 11 21 31 41 51 | |
| \| \| \| \| \| \| | |
| MPPFLLLEAV CVFLFSRVPP SLPLQEVHVS KETIGKISAA SKMMWCSAAV DIMFLLDGSN | 60 |
| SVGKGSFERS KHFAITVCDG LDISPERVRV GAFQFSSTPH LEFPLDSFST QQEVKARIKR | 120 |
| MVFKGGRTET ELALKYLLHR GLPGGRNASV PQILIIVTDG KSQGDVALPS KQLKERGVTV | 180 |
| FAVGVRFPRW EELHALASEP RGQHVLLAEQ VEDATNGLFS TLSSSAICSS ATPDCRVEAH | 240 |
| PCEHRTLEMV REFAGNAPCW RGSRRTLAVL AAHCPFYSWK RVFLTHPATC YRTTCPGPCD | 300 |
| SQPCQNGGTC VPEGLDGYQC LCPLAFGGEA NCALKLSLEC RVDLLFLLDS SAGTTLDGFL | 360 |
| RAKVFVKRFV RAVLSEDSRA RVGVATYSRE LLVAVPVGEY QDVPDLVWSL DGIPFRGGPT | 420 |
| LTGSALRQAA ERGFGSATRT GQDRPRRVVV LLTESHSEDE VAGPARHARA RELLLLGVGS | 480 |
| EAVRAELEEI TGSPKHVMVY SDPQDLFNQI PELQGKLCSR QRPGCRTQAL DLVFMLDTSA | 540 |
| SVGPENFAQM QSFVRSCALQ FEVNPDVTQV GLVVYGSQVQ TAFGLDTKPT RAAMLRAISQ | 600 |
| APYLGGVGSA GTALLHIYDK VMTVQRGARP GVPKAVVVLT GGRGAEDAAV PAQKLRNNGI | 660 |
| SVLVVGVGPV LSEGLRRLAG PRDSLIHVAA YADLRYHQDV LIEWLCGEAK QPVNLCKPSP | 720 |
| CMNEGSCVLQ NGSYRCKCRD GWEGPHCENR EWSSCSVCVS QGWILETPLR HMAPVQEGSS | 780 |
| RTPPSNYREG LGTEMVPTFW NVCAPGP | |
| Seq ID NO: 24 DNA sequence | |
| Nucleic Acid Accession #: see Table 25 & 25A for complete list | |
| 1 11 21 31 41 51 | |
| \| \| \| \| \| \| | |
| AGGTCGGCTG GTTATCGGGA GTTGGAGGGC TGAGGTCGGG AGGGTGGTGT GTACAGAGCT | 60 |
| CTAGGACTCA CGCACCAGGC CAGTCGCGGG TTTTGGGCCG AGGCCTGGGT TACAAGCAGC | 120 |
| AAGTGCGCGG TTGGGGCCAC TGCGAGGCCG TTTTAGAAAA CTGTTTAAAA CAAAGAGCAA | 180 |
| TTGATGGATA AATCAGGAAT AGATTCTCTT GACCATGTGA CATCTGATGC TGTGGAACTT | 240 |
| GCAAATCGAA GTGATAACTC TTCTGATAGC AGCTTATTTA AAACTCAGTG TATCCCTTAC | 300 |
| TCACCTAAAG GGGAGAAAAG AAACCCCATT CGAAAATTTG TTCGTACACC TGAAAGTGTT | 360 |
| CACGCAAGTA TTCATCAAGT GACTCATCTT TTGAACCAGT ACCATTGACT ATAAAAGCTA | 420 |
| TTTTTGAAAG ATTCAAGAAC AGGAAAAAGA GATATAAAAA AAAGAAAAAG AGGAGGTACC | 480 |
| AGCCAACAGG AAGACCACGG GGAAGACCAG AAGGAAGGAG AAATCCTATA TACTCACTAA | 540 |
| TAGATAAGAA GAAACAATTT AGAAGCAGAG GATCTGGCTT CCCATTTTTA GAATCAGAGA | 600 |
| ATGAAAAAAA CGCACCTTGG AGAAAAATTT TAACGTTTGA GCAAGCTGTT GCAAGAGGAT | 660 |
| TTTTTAACTA TATTGAAAAA CTGAAGTATG AACACCACCT GAAAGAATCA TTGAAGCAAA | 720 |
| TGAATGTTGG TGAAGATTTA GAAAATGAAG ATTTTGACAG TCGTAGATAC AAATTTTTGG | 780 |
| ATGATGATGG ATCCATTTCT CCTATTGAGG AGTCAACGCT TTTATCTTGA GGACATGGTG | 840 |
| TCTGGAGTTA AAGGTATTGG CATACTCCAC ACATCTGTAC CATTCTTGAG TGATCGCTTA | 900 |
| GGAATGAATG TGATTTGGAC TCATTCATGT ATGAGAGTAA GCAATGCTTT TTTTTCCAGG | 960 |
| GTGTCAAATT GAGAACCAGG TAGATCCCCA CCACCTACAG TAAAAAGGAC CCTAAAGTAA | 1020 |
| ATTGGTTGAA GAAATTAGAT CCCAAAGATT CTTGGTGAAT TTTGAAGTCT TCATCAGTAT | 1080 |
| ATCCATATTA AAACGAGATG ACAGAAGCCA AAGTAATTAT GGGCTGACAG GACAACTGGA | 1140 |
| TCAGTTTCAT TAAAAAGGGC AAACTTGAAG ATAAATCTTT TGACTCCAGC TCTTTAGAGG | 1200 |
| ATCTAAAGTG ACCTTGATGG ACAGTGGAAG AAATCACAAC ATGGAATTCC TCGAATAACA | 1260 |
| ATTTATTGAC TTTAAATAAT TTTGTCTAAT GCTACATATA CACAATTAAA AAACCTTTAC | 1320 |
| ACTATTTCTA GAAAGTCAGC ATGTATTTTT GGCTCGAAGT TTCTCTAGTT TTTTCTGTGG | 1380 |
| AAGGAATAAA AATTTGAGGT TTCAATACAA AAACAAAACA AACAACACGA AACACGAAAA | 1440 |
| ACAATCTGTT GTGCGGCGCC CCTGGGCCCC TTGAGAGAAA ACTTTTTAGA ACCCCTTTTG | 1500 |
| CGTTGTGGCG GCCCGGGGGC CCCACAGTTG GGTTTAGGTG GGCACCCTTG TGTCTACAAG | 1560 |
| TGGTGTCTCC CCAAGAGAGA GAACACCTCC GGGGTCAAGC GGACAACAAG AGTGCGTCGT | 1620 |
| GAGGACTCTT CACCCAAAGT ATATAAAACC CGCCCCGCGG GGGAACCACC GGCCGCTTTT | 1680 |
| CTGTAGACAC AACCCCCACA GTGGGAACCT CTGAGGGCGC ACACACAGGG CGAGCCTTAT | 1740 |
| CAACAAGGGG TGCCCAACAG AAACCCCGAG TTAAAAATCG | |
| Seq ID NO: 25 DNA sequence | |
| Nucleic Acid Accession #: BC001972.1 | |
| Coding sequence: 183-1019 | |
| 1 11 21 31 41 51 | |
| \| \| \| \| \| \| | |
| GGTCGGCTGG TTATCGGGAG TTGGAGGGCT GAGGTCGGGA GGGTGGTGTG TACAGAGCTC | 60 |
| TAGGACTCAC GCACCAGGCC AGTCGCGGGT TTTGGGCCGA GGCCTGGGTT ACAAGCAGCA | 120 |
| AGTGCGCGGT TGGGGCCACT GCGAGGCCGT TTTAGAAAAC TGTTTAAAAC AAAGAGCAAT | 180 |
| TGATGGATAA ATCAGGAATA GATTCTCTTG ACCATGTGAC ATCTGATGCT GTGGAACTTG | 240 |
| CAAATCGAAG TGATAACTCT TCTGATAGCA GCTTATTTAA AACTCAGTGT ATCCCTTACT | 300 |
| CACCTAAAGG GGAGAAAAGA AACCCCATTC GAAAATTTGT TCGTACACCT GAAAGTGTTC | 360 |
| ACGCAAGTGA TTCATCAAGT GACTCATCTT TTGAACCAAT ACCATTGACT ATAAAAGCTA | 420 |
| TTTTTGAAAG ATTCAAGAAC AGGAAAAAGA GATATAAAAA AAAGAAAAAG AGGAGGTACC | 480 |
| AGCCAACAGG AAGACCACGG GGAAGACCAG AAGGAAGGAG AAATCCTATA TACTCACTAA | 540 |
| TAGATAAGAA GAAACAATTT AGAAGCAGAG GATCTGGCTT CCCATTTTTA GAATCAGAGA | 600 |
| ATGAAAAAAA CGCACCTTGG AGAAAAATTT TAACGTTTGA GCAAGCTGTT GCAAGAGGAT | 660 |
| TTTTTAACTA TATTGAAAAA CTGAAGTATG AACACCACCT GAAAGAATCA TTGAAGCAAA | 720 |
| TGAATGTTGG TGAAGATTTA GAAAATGAAG ATTTTGACAG TCGTAGATAC AAATTTTTGG | 780 |
| ATGATGATGG ATCCATTTCT CCTATTGAGG AGTCAACAGC AGAGGATGAG GATGCAACAC | 840 |
| ATCTTGAAGA TAACGAATGT GATATCAAAT TGGCAGGGGA TAGTTTCATA GTAAGTTCTG | 900 |
| AATTCCCTGT AAGACTGAGT GTATACTTAG AAGAAGAGGA TATTACTGAA GAAGCTGCTT | 960 |
| TGTCTAAAAA GAGAGCTACA AAAGCCAAAA ATACTGGACA GAGAGGCCTG AAAATGTGAC | 1020 |
| AGGATCATGA ATGTCAAAGG CTTTTATCTT GAGAACATGG TGTCTGGAGT TAAAGGACTA | 1080 |
| TTGTTAGATC TGTGGGAAGG AATTACAAGA CAGTTGCTAA AAGTTTGAAA AAGACGGTTG | 1140 |
| CTAAACGTTA TGAAAAACCA GATAATCTAC TTTTTTACCT TAGGTATTGG CATACTCCAC | 1200 |
| ACATCTGTAC CATTCTTGAG TGATCGCTTA GGAATGAATG TGATTTGAAC TCATTCATGT | 1260 |
| TGAGAGGGTG TCAAATTGAG AACCAGGTAG ATCCCCACCA CCTACAGTAA AAAGGACCCT | 1320 |
| AAAGTAAATT GGTTGAAGAA ATTAGATCCC AAAGATTCTT GGTGAATTTT GAAGTCTTCA | 1380 |
| TCAGTATATC CATATTAAAA CGAGATGACA GAAGCCAAAG TAATTATGGG CTGACAGGAC | 1440 |
| AACTGGATCA GTTTCATTAA AAAGGGCAAA CTTGAAGATA AATCTTTTGA CTCCAGCTCT | 1500 |
| TTAGAGGATC TAAAGTGACC TTGATGGACA GTGGAAGAAA TCACAACATG GAATTCCTCG | 1560 |
| AATAACAATT TATTGACTTT AAATAATTTT GTCTAATGCT ACATATACAC AATTAAAAAA | 1620 |
| CCTTTACACT AAAAAAAAAA AAAAAA | |
| Seq ID NO: 26 Protein sequence: | |
| Protein Accession #: AAH01972.1 | |
| 1 11 21 31 41 51 | |
| \| \| \| \| \| \| | |
| MDKSGIDSLD HVTSDAVELA NRSDNSSDSS LFKTQCIPYS PKGEKRNPIR KFVRTPESVH | 60 |
| ASDSSSDSSF EPIPLTIKAI FERFKNRKKR YKKKKKRRYQ PTGRPRGRPE GRRNPIYSLI | 120 |
| DKKKQFRSRG SGFPFLESEN EKNAPWRKIL TFEQAVARGF FNYIEKLKYE HHLKESLKQM | 180 |
| NVGEDLENED FDSRRYKFLD DDGSISPIEE STAEDEDATH LEDNECDIKL AGDSFIVSSE | 240 |
| FPVRLSVYLE EEDITEEAAL SKKRATKAKN TGQRGLKM | |
| Seq ID NO: 27 DNA sequence | |
| Nucleic Acid Accession #: AK027016 | |
| Coding sequence: 207-1043 | |
| 1 11 21 31 41 51 | |
| \| \| \| \| \| \| | |
| CTTTTCTTCC GCACGGTTGG AGGAGGTCGG CTGGTTATCG GGAGTTGGAG GGCTGAGGTC | 60 |
| GGGAGGGTGG TGTGTACAGA GCTCTAGGAC TCACGCACCA GGCCAGTCGC GGATTTTGGG | 120 |
| CCGAGGCCTG GGTTACAAGC AGCAAGTGCG CGGTTGGGGC CACTGCGAGG CCGTTTTAGA | 180 |
| AAACTGTTTA AAACAAAGAG CAATTGATGG ATAAATCAGG AATAGATTCT CTTGACCATG | 240 |
| TGACATCTGA TGCTGTGGAA CTTGCAAATC GAAGTGATAA CTCTTCTGAT AGCAGCTTAT | 300 |
| TTAAAACTCA GTGTATCCCT TACTCACCTA AAGGGGAGAA AAGAAACCCC ATTCGAAAAT | 360 |
| TTGTTCGTAC ACCTGAAAGT GTTCACGCAA GTGATTCATC AAGTGACTCA TCTTTTGAAC | 420 |
| CAATACCATT GACTATAAAA GCTATTTTTG AAAGATTCAA GAACAGGAAA AAGAGATATA | 480 |
| AAAAAAAGAA AAAGAGGAGG TACCAGCCAA CAGGAAGACC ACGGGGAAGA CCAGAAGGAA | 540 |
| GGAGAAATCC TATATACTCA CTAATAGATA AGAAGAAACA ATTTAGAAGC AGAGGATCTG | 600 |
| GCTTCCCATT TTTAGAATCA GAGAATGAAA AAAACGCACC TTGGAGAAAA ATTTTAACGT | 660 |
| TTGAGCAAGC TGTTGCAAGA GGATTTTTTA ACTATATTGA AAAGCTGAAG TATGAACACC | 720 |
| ACCTGAAAGA ATCATTGAAG CAAATGAATG TTGGTGAAGA TTTAGAAAAT GAAGATTTTG | 780 |
| ACAGTCGTAG ATACAAATTT TTGGATGATG ATGGATCCAT TTCTCCTATT GAGGAGTCAA | 840 |
| CAGCAGAGGA TGAGGATGCA ACACATCTTG AAGATAACGA ATGTGATATC AAATTGGCAG | 900 |
| GGGATAGTTT CATAGTAAGT TCTGAATTCC CTGTAAGACT GAGTGTATAC TTAGAAGAAG | 960 |
| AGGATATTAC TGAAGAAGCT GCTETGTCTA AAAAGAGAGC TACAAAAGCC AAAAATACTG | 1020 |
| GACAGAGAGG CCTGAAAATG TGACAGGATC ATGAATGTCA AAGGCTTTTA TCTTGAGAAC | 1080 |
| ATGGTGTCTG GAGTTAAAGG TATTGGCATA CTCCACACAT CTGTACCATT CTTGAGTGAT | 1140 |
| CGCTTAGGAA TGAATGTGAT TTGAACTCAT TCATGTTGAG AGGGTGTCAA ATTGAGAACC | 1200 |
| AGGTAGATCC CCACCACCTA CAGTAAAAAG GACCCTAAAG TAAATTGGTT GAAGAAATTA | 1260 |
| GATCCCAAAG ATTCTTGGTG AATTTTGAAG TCTTCATCAG TATATCCATA TTAAAACGAG | 1320 |
| ATGACAGAAG CCAAAGTAAT TATGGCAAGT AATGGTTTTT ATCTTAACTA TAAGTTATTT | 1380 |
| GCTCAAGGGT GTAATGGTCA TTACCAAGGC TTTTAGAATG CAGTTTCTCA TTTGCTGTGG | 1440 |
| ACATGACCAT AAAAAAAAAT TTCCCAGTAG GTTTTCTATC TGCTACGTTG CTAGCAATCA | 1500 |
| GCTTATTGGG AACAGTTGAT TAACTGTAAT AGAAATGCAA TACAAATAAA ATGTGAACCA | 1560 |
| CATGTGATTT TTCTTTAAAA TCAGTGAGAT TTGAAAATTC TCCTAGATCT CTTGAATCAT | 1620 |
| GCAAATTTGC TTTGCCTTTA TATTGTAACC CTTGTGGGTT GCTAATAACC AAGCAGTTTG | 1680 |
| TAGTAGAGTT AACTCAGGCT CGTTCTAGGG ACTCATTCAT GTTCACTCAC TGTACACTCA | 1740 |
| TCTCTGGAAA TGTAAAATTT ACTTTTATAC TATTGTTATG TAGGGCTGAC AGGACAACTG | 1800 |
| GATCAGTTTC ATTAAAAAGG TATGTATGCA TTAGAAAAGA CATTTGTATG GGTCATTTCA | 1860 |
| AAGAGGGCTT ATGAGGCTGT GAAACCCAGA GCTCTTAACG CTGTGACCAA AGATGGAAGT | 1920 |
| TCTCTATAGG AAGCCATAGC ACTCCTAATG TTTGGTGCTA TGTTTTCCTG AGGAGATATA | 1980 |
| AAACGTAATA ATCCATGATT GTTGCCATGT GAGAGTTTTA AAGGTTAATC AAAATTTCTC | 2040 |
| TTCTTCAGGG CAAACTTGAA GATAAATCTT TTGACTCCAG CTCTTTAGAG GATCTAAAGT | 2100 |
| GACCTTGATG GACAGTGGAA GAAATCACAA CATGGAATTC CTCGAATAAC AATTTATTGA | 2160 |
| CTTTAAATAA TTTTGTCTAA TGCTACATAT ACACAATTAA AAAACCTTTA CACTATTTCT | 2220 |
| AGAAAGTCAG CATGTATTTT TGGCTCGAAG TTTCTCTAGT GTTTTCTGTG GAAGGAATAA | 2280 |
| AAATTTGAGT TTCAAAAAAA AAAAAAAAAA AAAAAAAAAA AAAAAAAAAA AAAAAA | |
| Seq ID NO: 28 Protein sequence: | |
| Protein Accession #: BAB15628.1 | |
| 1 11 21 31 41 51 | |
| \| \| \| \| \| \| | |
| MDKSGIDSLD HVTSDAVELA NRSDNSSDSS LFKTQCIPYS PKGEKRNPIR KFVRTPESVH | 60 |
| ASDSSSDSSF EPIPLTIKAI FERFKNRKKR YKKKKKRRYQ PTGRPRGRPE GRRNPIYSLI | 120 |
| DKKKQFRSRG SGFPFLESEN EKNAPWRKIL TFEQAVARGF FNYIEKLKYE HHLKESLKQM | 180 |
| NVGEDLENED FDSRRYKFLD DDGSISPIEE STAEDEDATH LEDNECDIKL AGDSFIVSSE | 240 |
| FPVRLSVYLE EEDITEEAAL SKKRATKAKN TGQRGLKM | |
| Seq ID NO: 29 DNA sequence | |
| Nucleic Acid Accession #: NM_004289.3 | |
| Coding sequence: 493.1695 | |
| 1 11 21 31 41 51 | |
| \| \| \| \| \| \| | |
| GCCGCCGCCT CGTCCACCGG AGGAGCCGGC GCCAGCGTGG ACGGCGGCAG CCAGGCTGTG | 60 |
| CAGGGGGGCG GCGGGGACCC CCGAGCGGCT CGGAGTGGCC CCTTGGACGC CGGGGAAGAG | 120 |
| GAGAAGGCAC CCGCGGAACC GACGGCTCAG GTGCCGGACG CTGGCGGATG TGCGAGCGAG | 180 |
| GAGAATGGGG TACTAAGAGA AAAGCACGAA GCTGTGGATC ATAGTTCCCA GCATGAGGAA | 240 |
| AATGAAGAAA GGGTGTCAGC CCAGAAGGAG AACTCACTTC AGCAGAATGA TGATGATGAA | 300 |
| AACAAAATAG CAGAGAAACC TGACTGGGAG GCAGAAAAGA CCACTGAATC TAGAAATGAG | 360 |
| AGACATCTGA ATGGGACAGA TACTTCTTTC TCTCTGGAAG ACTTATTCCA GTTGCTTTCA | 420 |
| TCACAGCCTG AAAATTCACT GGAGGGCATC TCATTGGGAG ATATTCCTCT TCCAGGCAGT | 480 |
| ATCAGTGATG GCATGAATTC TTCAGCACAT TATCATGTAA ACTTCAGCCA GGCTATAAGT | 540 |
| CAGGATGTGA ATCTTCATGA GGCCATCTTG CTTTGTCCCA ACAATACATT TAGAAGAGAT | 600 |
| CCAACAGCAA GGACTTCACA GTCACAAGAA CCATTTCTGC AGTTAAATTC TCATACCACC | 660 |
| AATCCTGAGC AAACCCTTCC TGGAACTAAT TTGACAGGAT TTCTTTCACC GGTTGACAAT | 720 |
| CATATGAGGA ATCTAACAAG CCAAGACCTA CTGTATGACC TTGACATAAA TATATTTGAT | 780 |
| GAGATAAACT TAATGTCATT GGCCACAGAA GACAACTTTG ATCCAATCGA TGTTTCTCAG | 840 |
| CTTTTTGATG AACCAGATTC TGATTCTGGC CTTTCTTTAG ATTCAAGTCA CAATAATACC | 900 |
| TCTGTCATCA AGTCTAATTC CTCTCACTCT GTGTGTGATG AAGGTGCTAT AGGTTATTGC | 960 |
| ACTGACCATG AATCTAGTTC CCATCATGAC TTAGAAGGTG CTGTAGGTGG CTACTACCCA | 1020 |
| GAACCCAGTA AGCTTTGTCA CTTGGATCAA AGTGATTCTG ATTTCCATGG AGATCTTACA | 1080 |
| TTTCAACACG TATTTCATAA CCACACTTAC CACTTACAGC CAACTGCACC AGAATCTACT | 1140 |
| TCTGAACCTT TTCCGTGGCC TGGGAAGTCA CAGAAGATAA GGAGTAGATA CCTTGAAGAC | 1200 |
| ACAGATAGAA ACTTGAGCCG TGATGAACAG CGTGCTAAAG CTTTGCATAT CCCTTTTTCT | 1260 |
| GTAGATGAAA TTGTCGGCAT GCCTGTTGAT TCTTTCAATA GCATGTTAAG TAGATATTAT | 1320 |
| CTGACAGACC TACAAGTCTC ACTTATCCGT GACATCAGAC GAAGAGGGAA AAATAAAGTT | 1380 |
| GCTGCGCAGA ACTGTCGTAA ACGCAAATTG GACATAATTT TGAATTTAGA AGATGATGTA | 1440 |
| TGTAACTTGC AAGCAAAGAA GGAAACTCTT AAGAGAGAGC AAGCACAATG TAACAAAGCT | 1500 |
| ATTAACATAA TGAAACAGAA ACTGCATGAC CTTTATCATG ATATTTTTAG TAGATTAAGA | 1560 |
| GATGACCAAG GTAGGCCAGT CAATCCCAAC CACTATGCTC TCCAGTGTAC CCATGATGGA | 1620 |
| AGTATCTTGA TAGTACCCAA AGAACTGGTG GCCTCAGGCC ACAAAAAGGA AACCCAAAAG | 1680 |
| GGAAAGAGAA AGTGAGAAGA AACTGAAGAT GGACTCTATT ATGTGAAGTA GTAATGTTCA | 1740 |
| GAAACTGATT ATTTGGATCA GAAACCATTG AAACTGCTTC AAGAATTGTA TCTTTAAGTA | 1800 |
| CTGCTACTTG AATAACTCAG TTAACGCTGT TTTGAAGCTT ACATGGACAA ATGTTTAGGA | 1860 |
| CTTCAAGATC ACACTTGTGG GCAATCTGGG GGAGCCACAA CTTTTCATGA AGTGCATTGT | 1920 |
| ATACAAAATT CATAGTTATG TCCAAAGAAT AGGTTAACAT GAAAACCCAG TAAGACTTTC | 1980 |
| CATCTTGGCA GCCATCCTTT TTAAGAGTAA GTTGGTTACT TCAAAAAGAG CAAACACTGG | 2040 |
| GGATCAAATT ATTTTAAGAG GTATTTCAGT TTTAAATGCA AAATAGCCTT ATTTTCATTT | 2100 |
| AGTTTGTTAG CACTATAGTG AGCTTTTCAA ACACTATTTT AATCTTTATA TTTAACTTAT | 2160 |
| AAATTTTGCT TTCT | |
| Seq ID NO: 30 Protein sequence: | |
| Protein Accession #: NP_004280 | |
| 1 11 21 31 41 51 | |
| \| \| \| \| \| \| | |
| MNSSAHYHVN FSQAISQDVN LHEAILLCPN NTFRRDPTAR TSQSQEPFLQ LNSHTTNPEQ | 60 |
| TLPGTNLTGF LSPVDNHMRN LTSQDLLYDL DINIFDEINL MSLATEDNFD PIDVSQLFDE | 120 |
| PDSDSGLSLD SSHNNTSVIK SNSSHSVCDE GAIGYCTDHE SSSHHDLEGA VGGYYPEPSK | 180 |
| LCHLDQSDSD FHGDLTFQHV FHNHTYHLQP TAPESTSEPF PWPGKSQKIR SRYLEDTDRN | 240 |
| LSRDEQRAKA LHIPFSVDEI VGMPVDSFNS MLSRYYLTDL QVSLIRDIRR RGKNKVAAQN | 300 |
| CRKRKLDIIL NLEDDVCNLQ AKKETLKREQ AQCNKAINIM KQKLHDLYHD IFSRLRDDQG | 360 |
| RPVNPNHYAL QCTHDGSILI VPKELVASGH KKETQKGKRK | |
| Seq ID NO: 31 DNA sequence | |
| Nucleic Acid Accession #: NM_033260.1 | |
| Coding sequence: 1-1208 | |
| 1 11 21 31 41 51 | |
| \| \| \| \| \| \| | |
| ATGAAGTTGG AGGTGTTCGT CCCTCGCGCG GCCCACGGGG ACAAGCAGGG CAGTGACCTG | 60 |
| GAGGGCGCGG GCGGCAGCGA CGCGCCGTCC CCGCTGTCGG CGGCGGGAGA CGACTCCCTG | 120 |
| GGCTCAGATG GGGACTGCGC GGCCAAGCCG TCCGCGGGCG GCGGCGCCAG AGATACGCAG | 180 |
| GGCGACGGCG AACAGAGTGC GGGAGGCGGG CCGGGCGCGG AGGAGGCGAT CCCGGCAGCA | 240 |
| GCTGCTGCAG CGGTGGTGGC GGAGGGCGCG GAGGCCGGGG CGGCGGGGCC AGGCGCGGGC | 300 |
| GGCGCGGGGA GCGGCGAGGG TGCACGCAGC AAGCCATATA CGCGGCGGCC CAAGCCCCCC | 360 |
| TACTCGTACA TCGCGCTCAT CGCCATGGCC ATCCGCGACT CGGCGGGCGG GCGCTTGACG | 420 |
| CTGGCGGAGA TCAACGAGTA CCTCATGGGC AAGTTCCCCT TTTTCCGCGG CAGCTACACG | 480 |
| GGCTGGCGCA ACTCCGTGCG CCACAACCTT TCGCTCAACG ACTGCTTCGT CAAGGTGCTG | 540 |
| CGCGACCCCT CGCGGCCCTG GGGCAAGGAC AACTACTGGA TGCTCAACCC CAACAGCGAG | 600 |
| TACACCTTCG CCGACGGGGT CTTCCGCCGC CGCCGCAAGC GCCTCAGCCA CCGCGCGCCG | 660 |
| GTCCCCGCGC CCGGGCTGCG GCCCGAGGAG GCCCCGGGCC TCCCCGCCGC CCCGCCGCCC | 720 |
| GCGCCCGCCG CCCCGGCCTC GCCCCGCATG CGCTCGCCCG CCCGCCAGGA GGAGCGCGCC | 780 |
| AGCCCCGCGG GCAAGTTCTC CAGCTCCTTC GCCATCGACA GCATCCTGCG CAAGCCTTTC | 840 |
| CGCAGCCGTC GCCTCAGGGA CACGGCCCCC GGGACGACGC TTCAGTGGGG CGCCGCGCCC | 900 |
| TGCCCGCCGC TGCCCGCGTT CCCCGCGCTC CTCCCCGCGG CGCCCTGCAG GGCCCTGCTG | 960 |
| CCGCTCTGCG CGTACGGCGC GGGCGAGCCG GCGCGGCTGG GCGCGCGCGA GGCCGAGGTG | 1020 |
| CCACCGACCG CGCCGCCCCT CCTGCTTGCA CCTCTCCCGG CGGCGGCCCC CGCCAAGCCA | 1080 |
| CTCCGAGGCC CGGCGGCCGG CGGCGCGCAC CTGTACTGCC CCCTGCGGCT GCCCGCAGCC | 1140 |
| CTGCAGGCGG CCTTAGTCCG NCGTCCTGGC CCGCACCTGT CGTACCCGGT GGAGACGCTC | 1200 |
| CTAGCTTGA | |
| Seq ID NO: 32 Protein sequence: | |
| Protein Accession #: NP_150285.1 | |
| 1 11 21 31 41 51 | |
| \| \| \| \| \| \| | |
| MKLEVFVPRA AHGDKQGSDL EGAGGSDAPS PLSAAGDDSL GSDGDCAAKP SAGGGARDTQ | 60 |
| GDGEQSAGGG PGAEEAIPAA AAAAVVAEGA EAGAAGPGAG GAGSGEGARS KPYTRRPKPP | 120 |
| YSYIALIAMA IRDSAGGRLT LAEINEYLMG KFPFFRGSYT GWRNSVRHNL SLNDCFVKVL | 180 |
| RDPSRPWGKD NYWMLNPNSE YTFADGVFRR RRKRLSHRAP VPAPGLRPEE APGLPAAPPP | 240 |
| APAAPASPRM RSPARQEERA SPAGKFSSSF AIDSILRKPF RSRRLRDTAP GTTLQWGAAP | 300 |
| CPPLPAFPAL LPAAPCRALL PLCAYGAGEP ARLGAREAEV PPTAPPLLLA PLPAAAPAKP | 360 |
| LRGPAAGGAH LYCPLRLPAA LQAALVRRPG PHLSYPVETL LA | |
| Seq 0) NO: 33 DNA sequence | |
| Nucleic Acid Accession #: NM_012128.2 | |
| Coding sequence: 43-2796 | |
| 1 11 21 31 41 51 | |
| \| \| \| \| \| \| | |
| GAACAAACCA ACATTTGAGC CAGGAATAAC TAGAGAGGAA CAATGGGGTT ATTCAGAGGT | 60 |
| TTTGTTTTCC TCTTAGTTCT GTGCCTGCTG CACCAGTCAA ATACTTCCTT CATTAAGCTG | 120 |
| AATAATAATG GCTTTGAAGA TATTGTCATT GTTATAGATC CTAGTGTGCC AGAAGATGAA | 180 |
| AAAATAATTG AACAAATAGA GGATATGGTG ACTACAGCTT CTACGTACCT GTTTGAAGCC | 240 |
| ACAGAAAAAA GATTTTTTTT CAAAAATGTA TCTATATTAA TTCCTGAGAA TTGGAAGGAA | 300 |
| AATCCTCAGT ACAAAAGGCC AAAACATGAA AACCATAAAC ATGCTGATGT TATAGTTGCA | 360 |
| CCACCTACAC TCCCAGGTAG AGATGAACCA TACACCAAGC AGTTCACAGA ATGTGGAGAG | 420 |
| AAAGGCGAAT ACATTCACTT CACCCCTGAC CTTCTACTTG GAAAAAAACA AAATGAATAT | 480 |
| GGACCACCAG GCAAACTGTT TGTCCATGAG TGGGCTCACC TCCGGTGGGG AGTGTTTGAT | 540 |
| GAGTACAATG AAGATCAGCC TTTCTACCGT GCTAAGTCAA AAAAAATCGA AGCAACAAGG | 600 |
| TGTTCCGCAG GTATCTCTGG TAGAAATAGA GTTTATAAGT GTCAAGGAGG CAGCTGTCTT | 660 |
| AGTAGAGCAT GCAGAATTGA TTCTACAACA AAACTGTATG GAAAAGATTG TCAATTCTTT | 720 |
| CCTGATAAAG TACAAACAGA AAAAGCATCC ATAATGTTTA TGCAAAGTAT TGATTCTGTT | 780 |
| GTTGAATTTT GTAACGAAAA AACCCATAAT CAAGAAGCTC CAAGCCTACA AAACATAAAG | 840 |
| TGCAATTTTA GAAGTACATG GGAGGTGATT AGCAATTCTG AGGATTTTAA AAACACCATA | 900 |
| CCCATGGTGA CACCACCTCC TCCACCTGTC TTCTCATTGC TGAAGATCCG TCAAAGAATT | 960 |
| GTGTGCTTAG TTCTTGATAA GTCTGGAAGC ATGGGGGGTA AGGACCGCCT AAATCGAATG | 1020 |
| AATCAAGCAG CAAAACATTT CCTGCTGCAG ACTGTTGAAA ATGGATCCTG GGTGGGGATG | 1080 |
| GTTCACTTTG ATAGTACTGC CACTATTGTA AATAAGCTAA TCCAAATAAA AAGCAGTGAT | 1140 |
| GAAAGAAACA CACTCATGGC AGGATTACCT ACATATCCTC TGGGAGGAAC TTCCATCTGC | 1200 |
| TCTGGAATTA AATATGCATT TCAGGTGATT GGAGAGCTAC ATTCCCAACT CGATGGATCC | 1260 |
| GAAGTACTGC TGCTGACTGA TGGGGAGGAT AACACTGCAA GTTCTTGTAT TGATGAAGTG | 1320 |
| AAACAAAGTG GGGCCATTGT TCATTTTATT GCTTTGGGAA GAGCTGCTGA TGAAGCAGTA | 1380 |
| ATAGAGATGA GCAAGATAAC AGGAGGAAGT CATTTTTATG TTTCAGATGA AGCTCAGAAC | 1440 |
| AATGGCCTCA TTGATGCTTT TGGGGCTCTT ACATCAGGAA ATACTGATCT CTCCCAGAAG | 1500 |
| TCCCTTCAGC TCGAAAGTAA GGGATTAACA CTGAATAGTA ATGCCTGGAT GAACGACACT | 1560 |
| GTCATAATTG ATAGTACAGT GGGAAAGGAC ACGTTCTTTC TCATCACATG GAACAGTCTG | 1620 |
| CCTCCCAGTA TTTCTCTCTG GGATCCCAGT GGAACAATAA TGGAAAATTT CACAGTGGAT | 1680 |
| GCAACTTCCA AAATGGCCTA TCTCAGTATT CCAGGAACTG CAAAGGTGGG CACTTGGGCA | 1740 |
| TACAATCTTC AAGCCAAAGC GAACCCAGAA ACATTAACTA TTACAGTAAC TTCTCGAGCA | 1800 |
| GCAAATTCTT CTGTGCCTCC AATCACAGTG AATGCTAAAA TGAATAAGGA CGTAAACAGT | 1860 |
| TTCCCCAGCC CAATGATTGT TTACGCAGAA ATTCTACAAG GATATGTACC TGTTCTTGGA | 1920 |
| GCCAATGTGA CTGCTTTCAT TGAATCACAG AATGGACATA CAGAAGTTTT GGAACTTTTG | 1980 |
| GATAATGGTG CAGGCGCTGA TTCTTTCAAG AATGATGGAG TCTACTCCAG GTATTTTACA | 2040 |
| GCATATACAG AAAATGGCAG ATATAGCTTA AAAGTTCGGG CTCATGGAGG AGCAAACACT | 2100 |
| GCCAGGCTAA AATTACGGCC TCCACTGAAT AGAGCCGCGT ACATACCAGG CTGGGTAGTG | 2160 |
| AACGGGGAAA TTGAAGCAAA CCCGCCAAGA CCTGAAATTG ATGAGGATAC TCAGACCACC | 2220 |
| TTGGAGGATT TCAGCCGAAC AGCATCCGGA GGTGCATTTG TGGTATCACA AGTCCCAAGC | 2280 |
| CTTCCCTTGC CTGACCAATA CCCACCAAGT CAAATCACAG ACCTTGATGC CACAGTTCAT | 2340 |
| GAGGATAAGA TTATTCTTAC ATGGACAGCA CCAGGAGATA ATTTTGATGT TGGAAAAGTT | 2400 |
| CAACGTTATA TCATAAGAAT AAGTGCAAGT ATTCTTGATC TAAGAGACAG TTTTGATGAT | 2460 |
| GCTCTTCAAG TAAATACTAC TGATCTGTCA CCAAAGGAGG CCAACTCCAA GGAAAGCTTT | 2520 |
| GCATTTAAAC CAGAAAATAT CTCAGAAGAA AATGCAACCC ACATATTTAT TGCCATTAAA | 2580 |
| AGTATAGATA AAAGCAATTT GACATCAAAA GTATCCAACA TTGCACAAGT AACTTTGTTT | 2640 |
| ATCCCTCAAG CAAATCCTGA TGACATFGAT CCTACTCCTA CTCCTACTCC TACTCCTGAT | 2700 |
| AAAAGTCATA ATTCTGGAGT TAATATTTCT ACGCTGGTAT TGTCTGTGAT TGGGTCTGTT | 2760 |
| GTAATTGTTA ACTTTATTTT AAGTACCACC ATTTGAACCT TAACGAAGAA AAAAATCTTC | 2820 |
| AAGTAGACCT AGAAGAGAGT TTTAAAAAAC AAAACAATGT AAGTAAAGGA TATTTCTGAA | 2880 |
| TCTTAAAATT CATCCCATGT GTGATCATAA ACTCATAAAA ATAATTTTAA GATGTCGGAA | 2940 |
| AAGGATACTT TGATTAAATA AAAACACTCA TGGATATGTA AAAACTGTCA AGATTAAAAT | 3000 |
| TTAATAGTTT CATTTATTTG TTATTTTATT TGTAAGAAAT AGTGATGAAC AAAGATCCTT | 3060 |
| TTTCATACTG ATACCTGGTT GTATATTATT TGATGCAACA GTTTTCTGAA ATGATATTTC | 3120 |
| AAATTGCATC AAGAAATTAA AATCATCTAT CTGAGTAGTC AAAATACAAG TAAAGGAGAG | 3180 |
| CAAATAAACA ACATTTGGAA AAAAAAAAAA AAAAAAAA | |
| Seq ID NO: 34 Protein sequence: | |
| Protein Accession #: NP_036260.1 | |
| 1 11 21 31 41 51 | |
| \| \| \| \| \| \| | |
| MGLFRGFVFL LVLCLLHQSN TSFIKLNNNG FEDIVIVIDP SVPEDEKIIE QIEDMVTTAS | 60 |
| TYLFEATEKR FFFKNVSILI PENWKENPQY KRPKHENHKH ADVIVAPPTL PGRDEPYTKQ | 120 |
| FTECGEKGEY IHFTPDLLLG KKQNEYGPPG KLFVHEWAHL RWGVFDEYNE DQPFYRAKSK | 180 |
| KIEATRCSAG ISGRNRVYKC QGGSCLSRAC RIDSTTKLYG KDCQFFPDKV QTEKASIMFM | 240 |
| QSIDSVVEFC NEKTHNQEAP SLQNIKCNFR STWEVISNSE DFKNTIPMVT PPPPPVFSLL | 300 |
| KIRQRIVCLV LDKSGSMGGK DRLNRMNQAA KHFLLQTVEN GSWVGMVHFD STATIVNKLI | 360 |
| QIKSSDERNT LMAGLPTYPL GGTSICSGIK YAFQVIGELH SQLDGSEVLL LTDGEDNTAS | 420 |
| SCIDEVKQSG AIVHFIALGR AADEAVIEMS KITGGSHFYV SDEAQNNGLI DAFGALTSGN | 480 |
| TDLSQKSLQL ESKGLTLNSN AWMNDTVIID STVGKDTFFL ITWNSLPPSI SLWDPSGTIM | 540 |
| ENFTVDATSK MAYLSIPGTA KVGTWAYNLQ AKANPETLTI TVTSRAANSS VPPITVNAKM | 600 |
| NKDVNSFPSP MIVYAEILQG YVPVLGANVT AFIESQNGHT EVLELLDNGA GADSFKNDGV | 660 |
| YSRYFTAYTE NGRYSLKVRA HGGANTARLK LRPPLNRAAY IPGWVVNGEI EANPPRPEID | 720 |
| EDTQTTLEDF SRTASGGAFV VSQVPSLPLP DQYPPSQITD LDATVHEDKI ILTWTAPGDN | 780 |
| FDVGKVQRYI IRISASILDL RDSFDDALQV NTTDLSPKEA NSKESFAFKP ENISEENATH | 840 |
| IFIAIKSIDK SNLTSKVSNI AQVTLFIPQA NPDDIDPTPT PTPTPDKSHN SGVNISTLVL | 900 |
| SVIGSVVIVN FILSTTI | |
| Seq ID NO: 35 DNA sequence | |
| Nucleic Acid Accession #: NM_000901.1 | |
| Coding sequence: 217-3171 | |
| 1 11 21 31 41 51 | |
| \| \| \| \| \| \| | |
| CGCGGGAGCC AACTTCAGGC TGCTCAGAGG AAGCCCGTGC AGTCAGTCAC CTGGGTGCAA | 60 |
| GAGCGTTGCT GCCTCGGGCT CTCCCGCTGC AGGGAGAGCG GCACTCGCTG GCCTGGATGT | 120 |
| GGTTGGATTT AGGGGGGCTC CGCAGCAGGG GTTTCGTGGC GGTGGCAAGC GCTGCAACAG | 180 |
| GTAGACGGCG AGAGACGGAC CCCGGCCGAG GCAGGGATGG AGACCAAAGG CTACCACAGT | 240 |
| CTCCCTGAAG GTCTAGATAT GGAAAGACGG TGGGGTCAAG TTTCTCAGGC TGTGGAGCGT | 300 |
| TCTTCCCTGG GACCTACAGA GAGGACCGAT GAGAATAACT ACATGGAGAT TGTCAACGTA | 360 |
| AGCTGTGTTT CCGGTGCTAT TCCAAACAAC AGTACTCAAG GAAGCAGCAA AGAAAAACAA | 420 |
| GAACTACTCC CTTGCCTTCA GCAAGACAAT AATCGGCCTG GGATTTTAAC ATCTGATATT | 480 |
| AAAACTGAGC TGGAATCTAA GGAACTTTCA GCAACTGTAG CTGAGTCCAT GGGTTTATAT | 540 |
| ATGGATTCTG TAAGAGATGC TGACTATTCC TATGAGCAGC AGAACCAACA AGGAAGCATG | 600 |
| AGTCCAGCTA AGATTTATCA GAATGTTGAA CAGCTGGTGA AATTTTACAA AGGAAATGGC | 660 |
| CATCGTCCTT CCACTCTAAG TTGTGTGAAC ACGCCCTTGA GATCATTTAT GTCTGACTCT | 720 |
| GGGAGCTCCG TGAATGGTGG CGTCATGCGC GCCATTGTTA AAAGCCCTAT CATGTGTCAT | 780 |
| GAGAAAAGCC CGTCTGTTTG CAGCCCTCTG AACATGACAT CTTCGGTTTG CAGCCCTGCT | 840 |
| GGAATCAACT CTGTGTCCTC CACCACAGCC AGCTTTGGCA GTTTTCCAGT GCACAGCCCA | 900 |
| ATCACCCAGG GAACTCCTCT GACATGCTCC CCTAATGCTG AAAATCGAGG CTCCAGGTCG | 960 |
| CACAGCCCTG CACATGCTAG CAATGTGGGC TCTCCTCTCT CAAGTCCGTT AAGTAGCATG | 1020 |
| AAATCCTCAA TTTCCAGCCC TCCAAGTCAC TGCAGTGTAA AATCTCCAGT CTCCAGTCCC | 1080 |
| AATAATGTCA CTCTGAGATC CTCTGTGTCT AGCCCTGCAA ATATTAACAA CTCAAGGTGC | 1140 |
| TCTGTTTCCA GCCCTTCGAA CACTAATAAC AGATCCACGC TTTCCAGTCC GGCAGCCAGT | 1200 |
| ACTGTGGGAT CTATCTGTAG CCCTGTAAAC AATGCCTTCA GCTACACTGC TTCTGGCACC | 1260 |
| TCTGCTGGAT CCAGTACATT GCGGGATGTG GTTCCCAGTC CAGACACGCA GGAGAAAGGT | 1320 |
| GCTCAAGAGG TCCCTTTTCC TAAGACTGAG GAAGTAGAGA GTGCCATCTC AAATGGTGTG | 1380 |
| ACTGGCCAGC TTAATATTGT CCAGTACATA AAACCAGAAC CAGATGGAGC TTTTAGCAGC | 1440 |
| TCATGTCTAG GAGGAAATAG CAAAATAAAT TCGGATTCTT CATTCTCAGT ACCAATAAAG | 1500 |
| CAAGAATCAA CCAAGCATTC ATGTTCAGGC ACCTCTTTTA AAGGGAATCC AACAGTAAAC | 1560 |
| CCGTTTCCAT TTATGGATGG CTCGTATTTT TCCTTTATGG ATGATAAAGA CTATTATTCC | 1620 |
| CTATCAGGAA TTTTAGGACC ACCTGTGCCC GGCTTTGATG GTAACTGTGA AGGCAGCGGA | 1680 |
| TTCCCAGTGG GTATTAAACA AGAACCAGAT GACGGGAGCT ATTACCCAGA GGCCAGCATC | 1740 |
| CCTTCCTCTG CTATTGTTGG GGTGAATTCA GGTGGACAGT CCTTCCACTA CAGGATTGGT | 1800 |
| GCTCAAGGTA CAATATCTTT ATCACGATCG GCTAGAGACC AATCTTTCCA ACACCTGAGT | 1860 |
| TCCTTTCCTC CTGTCAATAC TTTAGTGGAG TCATGGAAAT CACACGGCGA CCTGTCGTCT | 1920 |
| AGAAGAAGTG ATGGGTATCC GGTCTTAGAA TACATTCCAG AAAATGTATC AAGCTCTACT | 1980 |
| TTACGAAGTG TTTCTACTGG ATCTTCAAGA CCTTCAAAAA TATGTTTGGT GTGTGGGGAT | 2040 |
| GAGGCTTCAG GATGCCATTA TGGGGTAGTC ACCTGTGGCA GCTGCAAAGT TTTCTTCAAA | 2100 |
| AGAGCAGTGG AAGGGCAACA CAACTATTTA TGTGCTGGAA GAAATGATTG CATCATTGAT | 2160 |
| AAGATTCGAC GAAAGAATTG TCCTGCTTGC AGACTTCAGA AATGTCTTCA AGCTGGAATG | 2220 |
| AATTTAGGAG CACGAAAGTC AAAGAAGTTG GGAAAGTTAA AAGGGATTCA CGAGGAGCAG | 2280 |
| CCACAGCAGC AGCAGCCCCC ACCCCCACCC CCACCCCCGC AAAGCCCAGA GGAAGGGACA | 2340 |
| ACGTACATCG CTCCTGCAAA AGAACCCTCG GTCAACACAG CACTGGTTCC TCAGCTCTCC | 2400 |
| ACAATCTCAC GAGCGCTCAC ACCTTCCCCC GTTATGGTCC TTGAAAACAT TGAACCTGAA | 2460 |
| ATTGTATATG CAGGCTATGA CAGCTCAAAA CCAGATACAG CCGAAAATCT GCTCTCCACG | 2520 |
| CTCAACCGCT TAGCAGGCAA ACAGATGATC CAAGTCGTGA AGTGGGCAAA GGTACTTCCA | 2580 |
| GGATTTAAAA ACTTGCCTCT TGAGGACCAA ATTACCCTAA TCCAGTATTC TTGGATGTGT | 2640 |
| CTATCATCAT TTGCCTTGAG CTGGAGATCG TACAAACATA CGAACAGCCA ATTTCTCTAT | 2700 |
| TTTGCACCAG ACCTAGTCTT TAATGAAGAG AAGATGCATC AGTCTGCCAT GTATGAACTA | 2760 |
| TGCCAGGGGA TGCACCAAAT CAGCCTTCAG TTCGTTCGAC TGCAGCTCAC CTTTGAAGAA | 2820 |
| TACACCATCA TGAAAGTTTT GCTGCTACTA AGCACAATTC CAAAGGATGG CCTCAAAAGC | 2880 |
| CAGGCTGCAT TTGAAGAAAT GAGGACAAAT TACATCAAAG AACTGAGGAA GATGGTAACT | 2940 |
| AAGTGTCCCA ACAATTCTGG GCAGAGCTGG CAGAGGTTCT ACCAACTGAC CAAGCTGCTG | 3000 |
| GACTCCATGC ATGACCTGGT GAGCGACCTG CTGGAATTCT GCTTCTACAC CTTCCGAGAG | 3060 |
| TCCCATGCGC TGAAGGTAGA GTTCCCCGCA ATGCTGGTGG AGATCATCAG CGACCAGCTG | 3120 |
| CCCAAGGTGG AGTCGGGGAA CGCCAAGCCG CTCTACTTCC ACCGGAAGTG ACTGCCCGCT | 3180 |
| GCCCAGAAGA ACTTTGCCTT AAGTTTCCCT GTGTTGTTCC ACACCCAGAA GGACCCAAGA | 3240 |
| AAACCTGTTT TTAACATGTG ATGGTTGATT CACACTTGTT CAACAGTTTC TCAAGTTTAA | 3300 |
| AGTCATGTCA GAGGTTTGGA GCCGGGAAAG CTGTTTTTCC GTGGATTTGG CGAGACCAGA | 3360 |
| GCAGTCTGAA GGATTCCCCA CCTCCAATCC CCCAGCGCTT AGAAACATGT TCCTGTTCCT | 3420 |
| CGGGATGAAA AGCCATATCT AGTCAATAAC TCTGATTTTG ATATTTTCAC AGATGGAAGA | 3480 |
| AGTTTTAACT ATGCCGTGTA GTTTCTGGTA TCGTTCGCTT GTTTTAAAAG GGTTCAAGGA | 3540 |
| CTAACGAACG TTTTAAAGCT TACCCTTGGT TTGCACATAA AACGTATAGT CAATATGGGG | 3600 |
| CATTAATATT CTTTTGTTAT TAAAAAAACA CAAAAAAATA ATAAAAAAAT ATATACAGAT | 3660 |
| TCCTGTTGTG TAATAACAGA ACTCGTGGCG TGGGGCAGCA GCTGCCTCTG AGCCCTCGCT | 3720 |
| CGTCCACGGT CTTCTGCATC ACTGGTATAC ACACTCGTTA GCGTCCATTT CTTATTTAAT | 3780 |
| TAGAATGGAT AAGATGATGT TAAATGCCTT GGTTTGATTT CTAGTATCTA TTGTGTTGGC | 3840 |
| TTTACAAATA ATTTTTTGCA GTCTTTTGCT GTGCTGTACA TTACTGTATG TATAAATTAT | 3900 |
| GAAGGACCTG AAATAAGGTA TAAGGATCTT TTGTAAATGA GACACATACA AAAAAAATCT | 3960 |
| TTAATGGTTA ATAGGATGAA TGGGAAAGTA TTTTTGAAAG AATTCTATTT TGCTGGAGAC | 4020 |
| TATTTAAGTA CTATCTTTGT CTAAACAAGG TAATTTTTTT TTGTAAAGTG CAATGTCCTG | 4080 |
| CATGCATAAT GAACCGTTTA CAGTGTATTT AAGAAAGGGA AAGCTGTGCC TTTTTTAGCT | 4140 |
| TCATATCTAA TTTACCATTA TTTTACAGTC TCTGTTGTAA ATAACCACAC TGAAACCTCT | 4200 |
| TCGGTTGTCT TGAAACCTTT CTACTTTTTC TGTACTTTTT GTTTTGTTCT TGGTCTCCCG | 4260 |
| CTTGGGGCAT TTGTGGGACT CCAGCACGTT TTCTGGCTTC TGCTTCATCC TGCTCCATCG | 4320 |
| GGGAATGACA CACTGCGGTG TCTGCAGCTC CTGGAAGGTG TCATTTGACA ACACATGTGG | 4380 |
| GAGAGGAGGT CCTTGGAGTG CTGCAGCTTT GGGAAAGCCT GCCTCGTTTC CCTTTTCCTC | 4440 |
| TAGAAGCAGA ACCAGCTCTA CGAGAGTGAG ACTGGGAACT TGATGGCTCA GAGAGCATCT | 4500 |
| TTTCCTCCCA TTTTAGAAAA TCAGATTTTC TCCTGTGGGA AAAAAAAATT CCATGCACTC | 4560 |
| TCTCTCTGTT AAAGATCAGC TATTCCCTTC TGATCTTGGA AAGAGGTTCT GCACTCCTGG | 4620 |
| AACCGGTCAC AGGAACGCAC AGATCATGGC AGGATGCGCT GGGACGGCCC ATCTTGGCAA | 4680 |
| GGTTCAGTCT GAATGGCATG GAGACCGGGA GATAGAGGGG TTTTAGATTT TTAAAAGGTA | 4740 |
| GGTTTTAAAA ATAAGTTTTA TACATAAACA GTTTTGGAGA AAAATTACAG ATCATATAAG | 4800 |
| CAAGACAGTG GCACTAAAAT GTTTAATTCA TTAATCTGTT TGTTTGGCAC TGATGCAATG | 4860 |
| TATGGCTTTT CTCTTGCCCC AAATCACAAA CATATGTATC TTTGGGGAAA CTAACAATAT | 4920 |
| GATTGCACTA AATAAACTAC TTTGAATAGA GGCCAAATTA ATCTTTTAAA AATGATGATA | 4980 |
| ATCATCAGGT TTACTCAGTG AAATCATATT AATTATTTTC CAAAATCTAA AAGCTGTAGC | 5040 |
| TGGAGAAGCC CATGGCCACG AGGAAGCAGC AATTAATTAG ATCAACACTT TTCTCCAGGG | 5100 |
| TTCACCATGC AGGCAACATT ACCTTGTCTT TCAAAAGACA CCTGCCTTAG TGCAAGGGGA | 5160 |
| AACCTGTGAA AGCTGCACTC AGAGGGAGGA GTCTTTCTTA CATAATTTGC AATTTCAGGA | 5220 |
| ATTTAATTTA TAGGCAGATC TTTAAATACA GTCAACTTAC GGTGCACAGT AATATGAAAG | 5280 |
| CCACACTTTG AAGGTAATAA ATACACAGCA TGCAGACTGG GAGTTGCTAG CAAACAAATG | 5340 |
| GCTTACTTAC AAAAGCAGCT TTTAGTTCAG ACTTAGTTTT TATAAAATGA GAATTCTGAC | 5400 |
| TTACTTAACC AGGTTTGGGA TGGAGATGGT CTGCATCAGC TTTTTGTATT AACAAAGTTA | 5460 |
| CTGGCTCTTT GTGTGTCTCC AGGTAACTTT GCTTGATTAA ACAGCAAAGC CATATTCTAA | 5520 |
| ATTCACTGTT GAATGCCTGT CCCAGTCCAA ATTGTCTGTC TGCTCTTATT TTTGTACCAT | 5580 |
| ATTGCTCTTA AAAATCTTGG TTTGGTACAG TTCATAATTC ACCAAAAAGT TCATATAATT | 5640 |
| TAAAGAAACA CTAAATTAGT TTAAAATGAA GCAATTTATA TCTTTATGCA AAAACATATG | 5700 |
| TCTGTCTTTG CAAAGGACTG TAAGCAGATT ACAATAAATC CTTTACTTT | |
| Seq ID NO: 36 Protein sequence: | |
| Protein Accession #: NP_000892.1 | |
| 1 11 21 31 41 51 | |
| \| \| \| \| \| \| | |
| METKGYHSLP EGLDMERRWG QVSQAVERSS LGPTERTDEN NYMEIVNVSC VSGAIPNNST | 60 |
| QGSSKEKQEL LPCLQQDNNR PGILTSDIKT ELESKELSAT VAESMGLYMD SVRDADYSYE | 120 |
| QQNQQGSMSP AKIYQNVEQL VKFYKGNGHR PSTLSCVNTP LRSFMSDSGS SVNGGVMRAI | 180 |
| VKSPIMCHEK SPSVCSPLNM TSSVCSPAGI NSVSSTTASF GSFPVHSPIT QGTPLTCSPN | 240 |
| AENRGSRSHS PAHASNVGSP LSSPLSSMKS SISSPPSHCS VKSPVSSPNN VTLRSSVSSP | 300 |
| ANINNSRCSV SSPSNTNNRS TKSSPAASTV GSICSPVNNA FSYTASGTSA GSSTLRDVVP | 360 |
| SPDTQEKGAQ EVPFPKTEEV ESAISNGVTG QLNIVQYIKP EPDGAFSSSC LGGNSKINSD | 420 |
| SSFSVPIKQE STKHSCSGTS FKGNPTVNPF PFMDGSYFSF MDDKDYYSLS GILGPPVPGF | 480 |
| DGNCEGSGFP VGIKQEPDDG SYYPEASIPS SAIVGVNSGG QSFHYRIGAQ GTISLSRSAR | 540 |
| DQSFQHLSSF PPVNTLVESW KSHGDLSSRR SDGYPVLEYI PENVSSSTLR SVSTGSSRPS | 600 |
| KICLVCGDEA SGCHYGVVTC GSCKVFFKRA VEGQHNYLCA GRNDCIIDKI RRKNCPACRL | 660 |
| QKCLQAGMNL GARKSKKLGK LKGIHEEQPQ QQQPPPPPPP PQSPEEGTTY IAPAKEPSVN | 720 |
| TALVPQLSTI SRALTPSPVM VLENIEPEIV YAGYDSSKPD TAENLLSTLN RLAGKQMIQV | 780 |
| VKWAKVLPGF KNLPLEDQIT LIQYSWMCLS SFALSWRSYK HTNSQFLYFA PDLVFNEEKM | 840 |
| HQSAMYELCQ GMHQISLQFV RLQLTFEEYT IMKVLLLLST IPKDGLKSQA AFEEMRTNYI | 900 |
| KELRKMVTKC PNNSGQSWQR FYQLTKLLDS MHDLVSDLLE FCFYTFRESH ALKVEFPAML | 960 |
| VEIISDQLPK VESGNAKPLY FHRK | |
| Seq ID NO: 37 DNA sequence | |
| Nucleic Acid Accession #: see Table 25 & 25A for complete list | |
| 1 11 21 31 41 51 | |
| \| \| \| \| \| \| | |
| CCTACCAGGT TCAAGCAACT CTGCTGCCTC AGCTCCCAAG TAGCTGGGAT TACAGGTGCA | 60 |
| TGCCACTACA CCTGGCTTTT TGTATTTTTA GTAGAGATGG TTTTCACTAT GTTGGCCAGG | 120 |
| CTGATCTTGA ATTCCTGGCC TGAAGTAATC TGCCTGCCTC AGCCTCCCAA AGTGCTGGGA | 180 |
| TTATAGGAGC CACCACACCT GGCATAACTG GTATTTTTTA TATGCTTCCT GGGCAACTTA | 240 |
| AAAAATTGAT TACTCTGTTG TTTCTTCCTT TTTTTTTTTT TTTTGGCTTT GACCAATTTG | 300 |
| TGAGACCCAA GTATCTCCTA CCTAGAAAAA AAACACACTA AACAGTAAAT GATTACCAAC | 360 |
| CTATTTGGAA CAAATCTCAA TTAATTAACA TATACTTCAA GGAGAAGACT TAACAAAATC | 420 |
| TTACTTTTCA TTCTTAATAG CTCTTTCCAT AAAAATGTTC CACAAGTGTA TCAAATTAGT | 480 |
| CCTAACAACT ACTGTTAAGT GATTAATGAA ACAGGAGTGA CAGGAGTGAA TTTAATAATA | 540 |
| GCAATAAATA CAGATGGGAC TACATAAATT GTGGAGGTCC TGATGCAAAA CTCTCTCTGT | 600 |
| ATTCGATGGC ATCTCAGCTT TCTCATAGAG CTGTTTCACT GTGAGGGTCT TTATCCTTCA | 660 |
| TGCAGAGCTT CATTATTTTC TTTCTTCTAG CAATCAGTCC AAAGCACAAT GTCAGAAAGA | 720 |
| TCACAACACA TGCAGCAATA ATGGGCTCTA TTGGTACACC CACAGTTTTA TCTTTAACAA | 780 |
| TC | |
| Seq ID NO: 38 DNA sequence | |
| Nucleic Acid Accession #: NM_001192.1 | |
| Coding sequence: 219-773 | |
| 1 11 21 31 41 51 | |
| \| \| \| \| \| \| | |
| AAGACTCAAA CTTAGAAACT TGAATTAGAT GTGGTATTCA AATCCITACG TGCCGCGAAG | 60 |
| ACACAGACAG CCCCCGTAAG AACCCACGAA GCAGGCGAAG TTCATTGTTC TCAACATTCT | 120 |
| AGCTGCTCTT GCTGCATTTG CTCTGGAATT CTTGTAGAGA TATTACTTGT CCTTCCAGGC | 180 |
| TGTTCTTTCT GTAGCTCCCT TGT3TTCTTT TTGTGATCAT GTTGCAGATG GCTGGGCAGT | 240 |
| GCTCCCAAAA TGAATATTTT GACAGTTTGT TGCATGCTTG CATACCTTGT CAACTTCGAT | 300 |
| GTTCTTCTAA TACTCCTCCT CTAACATGTC AGCGTTATTG TAATGCAAGT GTGACCAATT | 360 |
| CAGTGAAAGG AACGAATGCG ATTCTCTGGA CCTGTTTGGG ACTGAGCTTA ATAATTTCTT | 420 |
| TGGCAGTITT CGTGCTAATG TTTTTGCTAA GGAAGATAAG CTCTGAACCA TTAAAGGACG | 480 |
| AGTTTAAAAA CACAGGATCA GGTCTCCTGG GCATGGCTAA CATTGACCTG GAAAAGAGCA | 540 |
| GGACTGGTGA TGAAATTATT CTTCCGAGAG GCCTCGAGTA CACGGTGGAA GAATGCACCT | 600 |
| GTGAAGACTG CATCAAGAGC AAACCGAAGG TCGACTCTGA CCATTGCTTT CCACTCCCAG | 660 |
| CTATGGAGGA AGGCGCAACC ATTCTTGTCA CCACGAAAAC GAATGACTAT TGCAAGAGCC | 720 |
| TGCCAGCTGC TTTGAGTGCT ACGGAGATAG AGAAATCAAT TTCTGCTAGG TAATTAACCA | 780 |
| TTTCGAcTCG AGCAGTGCCA CTTTAAAAAT CTTTTGTCAG AATAGATGAT GTGTCAGATC | 840 |
| TCTTTAGGAT GACTGTATTT TTCAGTTGCC GATACAGCTT TTTGTCCTCT AACTGTGGAA | 900 |
| ACTCTTTATG TTAGATATAT TTCTCTAGGT TACTGTTGGG AGCTTAATGG TAGAAACTTC | 960 |
| CTTGGTTTCA TGATTAAAGT CTTTTTTTTT CCTGA | |
| Seq ID NO: 39 Protein sequence: | |
| Protein Accession #: NP_001183.1 | |
| 1 11 21 31 41 51 | |
| \| \| \| \| \| \| | |
| MLQMAGQCSQ NEYFDSLLHA CIPCQLRCSS NTPPLTCQRY CNASVTNSVK GTNAILWTCL | 60 |
| GLSLIISLAV FVLMFLLRKI SSEPLKDEFK NTGSGLLGMA NIDLEKSRTG DEIILPRGLE | 120 |
| YTVEECTCED CIKSKPKVDS DHCFPLPAME EGATILVTTK TNDYCKSLPA ALSATEIEKS | 180 |
| ISAR | |
| Seq ID NO: 40 DNA sequence | |
| Nucleic Acid Accession #: NM_025087.1 | |
| Coding sequence: 183-2282 | |
| 1 11 21 31 41 51 | |
| \| \| \| \| \| \| | |
| ACACTGCCTC GGTFCGGCAA GTGGGTCAGT TGGCTGGGGC TCACTTGGCA ACGGGACGCG | 60 |
| GGAACGAGGG GCGCGGACGC AGGCCCGGGA GGACGCGGCG GCGGGAACCT GGGGGCGCAG | 120 |
| GGCTAGGGCA GCGGGCCCGA CCCGCACGGC TTTCCTGGAA AGCGCTGCCC CTCGCCGCGG | 180 |
| CGATGACCTC GCTGTGGAGA GAAATCCTCT TGGAGTCGCT GCTGGGATGT GTTTCTTGGT | 240 |
| CTCTCTACCA TGACCTGGGA CCGATGATCT ATTACTTTCC TTTGCAAACA CTAGAACTCA | 300 |
| CTGGGCTTGA AGGTTTTAGT ATAGCATTTC TTTCTCCAAT ATTCCTAACA ATTACTCCTT | 360 |
| TCTCGAAATT GGTTAACAAG AAGTGGATGC TAACCCTGCT GAGGATAATC ACTATTGGCA | 420 |
| GCATAGCCTC CTTCCAGGCT CCAAATGCCA AACTTCGACT GATGGTTCTT GCGCTTGGGG | 480 |
| TGTCTTCCTC ACTGATAGTG CAAGCTGTGA CTTGGTGGTC AGGAAGTCAT TTGCAAAGGT | 540 |
| ACCTCAGAAT TTGGGGATTC ATTTTAGGAC AGATTGTTCT TGTTGTTCTA CGCATATGGT | 600 |
| ATACTTCACT AAACCCAATC TGGAGTTATC AGATGTCCAA CAAAGTGATA CTGACATTAA | 660 |
| GTGCCATAGC CACACTTGAT CGTATTGGCA CAGATGGTGA CTGCAGTAAA CCTGAAGAAA | 720 |
| AGAAGACTGG TGAGGTAGCC ACGGGGATGG CCTCTAGACC CAACTGGCTG CTGGCAGGGG | 780 |
| CTGCTTTTGG TAGCCTTGTG TTCCTCACCC ACTGGGTTTT TGGAGAAGTC TCTCTTGTTT | 840 |
| CCAGATGGGC AGTGAGTGGG CATCCACATC CAGGGCCAGA TCCTAACCCA TTTGGAGGTG | 900 |
| CAGTACTGCT GTGCTTGGCA AGTGGATTGA TGCTTCCATC TTGTTTGTGG TTTCGTGGTA | 960 |
| CTGGTTTGAT CTGGTGGGTT ACAGGAACAG CTTCAGCTGC GGGGCTCCTT TACCTGCACA | 1020 |
| CATGGGCAGC TGCTGTGTCT GGCTGTGTCT TCGCCATCTT TACTGCATCC ATGTGGCCCC | 1080 |
| AAACACTTGG ACACCTTATT AACTCAGGGA CAAACCCTGG GAAAACCATG ACCATTGCCA | 1140 |
| TGATATTTTA TCTTCTAGAA ATATTTTTCT GTGCCTGGTG CACAGCTTTT AAGTTTGTCC | 1200 |
| CAGGAGGTGT CTACGCTAGA GAAAGATCAG ATGTGCTTTT GGGGACAATG ATGTTAATTA | 1260 |
| TCGGGCTGAA TATGCTATTT GGTCCTAAGA AAAACCTTGA TTTGCTTCTT CAAACAAAAA | 1320 |
| ACAGTTCTAA AGTGCYTTTC AGAAAGAGTG AAAAATACAT GAAACTTTTT CTGTGGCTGC | 1380 |
| TTGTTGGTGT GGGATTGTTG GGATTAGGAC TACGGCATAA AGCCTATGAG AGAAAACTGG | 1440 |
| GCAAAGTGGC ACCAACCAAA GAGGTCTCTG CTGCCATCTG GCCTTTCAGG TTTGGATATG | 1500 |
| ACAATGAAGG GTGGTCTAGT CTAGAAAGAT CAGCTCACCT GCTCAATGAA ACAGGTGCAG | 1560 |
| ATTTCATAAC AATTTTGGAG AGTGATGCTT CTAAGCCCTA TATGGGGAAC AATGACTTAA | 1620 |
| CCATGTGGCT AGGGGAAAAG TTGGGTTTCT ATACAGACTT TGGTCCAAGC ACAAGGTATC | 1680 |
| ACACTTGGGG GATTATGGCT TTGTCAAGAT ACCCAATTGT GAAATCTGAG CATCACCTTC | 1740 |
| TTCCGTCACC AGAGGGCGAG ATCGCACCAG CCATCACATT GACCGTTAAC ATTTCGGGCA | 1800 |
| AGCTGGTGGA TTTTGTCGTG ACACACTTTG GGAACCACGA AGATGACCTC GACAGGAAAC | 1860 |
| TGCAGGCTAT TGCTGTTTCA AAACTACTGA AAAGTAGCTC TAATCAAGTG ATATTTCTGG | 1920 |
| GATATATCAC TTCAGCACCT GGCTCCAGAG ATTATCTACA GCTCACTGAA CATGGCAATG | 1980 |
| TGAAGGATAT CGACAGCACT GATCATGACA GATGGTGTGA ATACATTATG TATCGAGGGC | 2040 |
| TGATCAGGTT GGGTTATGCA AGAATCTCCC ATGCTGAACT GAGTGATTCA GAAATTCAGA | 2100 |
| TGGCAAAATT TAGGATCCCT GATGACCCCA CTAATTATAG AGACAACCAG AAAGTGGTCA | 2160 |
| TAGACCACAG AGAAGTTTCT GAGAAAATTC ATTTTAATCC CAGATTTGGA TCCTACAAAG | 2220 |
| AAGGACACAA TTATGAAAAC AACCATAATT TTCATATGAA TACTCCCAAA TACTTTTTAT | 2280 |
| GAAACATTTA AAACAAGAAG TTATTGGCTG GGAAAATCTA AGAAAAAAAG TATGTAAGAT | 2340 |
| AAAAAGAAGA GATTAATGAA AGTGGGAAAA TACACATGAA GAACCTCAAC TTAAAAAACA | 2400 |
| CATGGTATCT ATGCAGTGGG AAATTACCTC CATTTGTAAA CTATGTTGCT TAATAAAAAC | 2460 |
| ATTTCTCTAA AAAAAAAAAA AAAAAA | |
| Seq ID NO: 41 Protein sequence: | |
| Protein Accession #: NP_079363.1 | |
| 1 11 21 31 41 51 | |
| \| \| \| \| \| \| | |
| MTSLWREILL ESLLGCVSWS LYHDLGPMIY YFPLQTLELT GLEGFSIAFL SPIFLTITPF | 60 |
| WKLVNKKWML TLLRIITIGS IASFQAPNAK LRLMVLALGV SSSLIVQAVT WWSGSHLQRY | 120 |
| LRIWGFILGQ IVLVVLRIWY TSLNPIWSYQ MSNKVILTLS AIATLDRIGT DGDCSKPEEK | 180 |
| KTGEVATGMA SRPNWLLAGA AFGSLVFLTH WVFGEVSLVS RWAVSGHPHP GPDPNPFGGA | 240 |
| VLLCLASGLM LPSCLWFRGT GLIWWVTGTA SAAGLLYLHT WAAAVSGCVF AIFTASMWPQ | 300 |
| TLGHLINSGT NPGKTMTIAM IFYLLEIFFC AWCTAFKFVP GGVYARERSD VLLGTMMLII | 360 |
| GLNMLFGPKK NLDLLLQTKN SSKVLFRKSE KYMKLFLWLL VGVGLLGLGL RHKAYERKLG | 420 |
| KVAPTKEVSA AIWPFRFGYD NEGWSSLERS AHLLNETGAD FITILESDAS KPYMGNNDLT | 480 |
| MWLGEKLGFY TDFGPSTRYH TWGIMALSRY PIVKSEHHLL PSPEGEIAPA ITLTVNISGK | 540 |
| LVDFVVTHFG NHEDDLDRKL QAIAVSKLLK SSSNQVIFLG YITSAPGSRD YLQLTEHGNV | 600 |
| KDIDSTDHDR WCEYIMYRGL IRLGYARISH AELSDSEIQM AKFRIPDDPT NYRDNQKVVI | 660 |
| DHREVSEKIH FNPRFGSYKE GHNYENNHNF HMNTPKYFL | |

It is understood that the examples described above in no way serve to limit the true scope of this invention, but rather are presented for illustrative purposes. All publication, sequences of accession numbers, and patent applications cited in this specification are herein incorporated by reference as if each individual publication or patent application were specifically and individually indicated to be incorporated by reference.

What is claimed is:

1. A method of detecting a metastatic colorectal cancer-associated transcript in a cell from a patient, the method comprising contacting a biological sample from the patient with a polynucleotide that selectively hybridizes to a sequence at least 80% identical to a sequence as shown in Tables 1-26.

2. The method of claim 1, wherein the biological sample comprises isolated nucleic acids.

3. The method of claim 1, wherein the polynucleotide is labeled.

4. The method of claim 1, wherein the polynucleotide is immobilized on a solid surface.

5. An isolated nucleic acid molecule consisting of a polynucleotide sequence as shown in Tables 1-26.

6. An expression vector comprising the nucleic acid of claim 5.

7. A host cell comprising the expression vector of claim 6.

8. An isolated polypeptide which is encoded by a nucleic acid molecule having polynucleotide sequence as shown in Tables 1-26.

9. An antibody that specifically binds a polypeptide of claim 8.

10. The antibody of claim 10, which is an antibody fragment.

11. The antibody of claim 10, which is a humanized antibody

12. A method of detecting a metastatic colorectal cancer cell in a biological sample from a patient, the method comprising contacting the biological sample with an antibody of claim 9.

13. The method of claim 12, wherein the antibody is labeled.

14. A method of detecting antibodies specific to metastatic colorectal cancer in a patient, the method comprising contacting a biological sample from the patient with a polypeptide encoded by a nucleic acid comprises a sequence from Tables 1-26.

15. A method for identifying a compound that modulates a metastatic colorectal cancer-associated polypeptide, the method comprising the steps of:
(i) contacting the compound with a metastatic colorectal cancer-associated polypeptide, the polypeptide encoded by a polynucleotide that selectively hybridizes to a sequence at least 80% identical to a sequence as shown in Tables 1-26.; and
(ii) determining the functional effect of the compound upon the polypeptide.

16. The method of claim 15, wherein the functional effect is determined by measuring ligand binding to the polypeptide.

17. A method of inhibiting proliferation of a metastatic colorectal cancer-associated cell to treat colorectal cancer in a patient, the method comprising the step of administering to the subject a therapeutically effective amount of a compound that modulates a polypeptide encoded by a sequence as shown in Tables 1-26.

18. A drug screening assay comprising the steps of
(i) administering a test compound to a mammal having colorectal cancer or a cell isolated therefrom;
(ii) comparing the level of gene expression of a polynucleotide that selectively hybridizes to a sequence at least 80% identical to a sequence as shown in Tables 1-26. In a treated cell or mammal with the level of gene expression of the polynucleotide in a control cell or mammal, wherein a test compound that modulates the level of expression of the polynucleotide is a candidate for the treatment of colorectal cancer.

19. A pharmaceutical composition for treating a mammal having colorectal cancer, the composition comprising a compound identified by the assay of claim 18 and a physiologically acceptable excipient.

20. A method of detecting a metastatic colorectal cancer-associated polypeptide in a cell from a patient, the method comprising contacting a biological sample from the patient with a antibody that that specifically binds a polypeptide encoded by a nucleic acid molecule having polynucleotide sequence as shown in Tables 1-26.

21. The method of claim 21, wherein the antibody is labeled.
